(19) United States
(12) Patent Application Publication
(10) Pub. No.: US 2024/0341110 A1
(43) Pub. Date: Oct. 10, 2024

(54) PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Bitnari KIM, Gyeonggi-do (KR); Young-Jae KIM, Gyeonggi-do (KR); Hee-Ryong KANG, Gyeonggi-do (KR); Sang-Hee CHO, Gyeonggi-do (KR); So-Young JUNG, Gyeonggi-do (KR); Tae-Jun HAN, Gyeonggi-do (KR); Mi-Ran SEO, Gyeonggi-do (KR)

(21) Appl. No.: 18/441,092

(22) Filed: Feb. 14, 2024

(30) Foreign Application Priority Data
Mar. 23, 2023 (KR) 10-2023-0037814

(51) Int. Cl. H10K 50/11; H10K 85/60; H10K 101/00

(52) U.S. Cl. CPC H10K 50/11; H10K 85/622; H10K 85/633; H10K 85/636; H10K 85/654; H10K 85/6572; H10K 85/6574; H10K 85/6576; H10K 2101/90

(57) ABSTRACT

The present disclosure relates to a plurality of host materials comprising at least one first host compound represented by the following Formula 1 and at least one second host compound represented by the following Formula 2, and an organic electroluminescent device comprising the same. By comprising a specific combination of host materials according to the present disclosure in the light-emitting layer, an organic electroluminescent device exhibiting low driving voltage and/or high luminous efficiency and/or long lifespan characteristics can be manufactured.

### TECHNICAL FIELD

The present disclosure relates to a plurality of host materials and an organic electroluminescent device comprising the same.

### BACKGROUND ART

The TPD/Alq₃ bilayer small molecule organic electroluminescent device (OLED) with green-emission, which is constituted with a light-emitting layer and a charge transport layer, was first developed by Tang, et al., of Eastman Kodak in 1987. Thereafter, the studies on an OLED have been rapidly affected and OLEDs have been commercialized. At present, an organic electroluminescent device mainly includes phosphorescent materials having excellent luminous efficiency in panel realization. An OLED having high luminous efficiency and/or long lifespan characteristics is required for long-time use and a high-resolution display.

Various materials or concepts have been proposed for the organic layer of an organic electroluminescent device in order to improve luminous efficiency, driving voltage and/or lifespan, but they have not been satisfactory for practical use. Accordingly, there is a continuous need to develop organic electroluminescent devices with improved performance, such as improved driving voltage, luminous efficiency, power efficiency, and/or lifespan characteristics, compared to previously disclosed organic electroluminescent devices.

Korean Patent Application Laid-open No. 10-2020-0131681 discloses a host material for a phosphorescent light-emitting layer. However, said reference does not specifically disclose a plurality of host materials comprising the specific combination of compounds as described in the present disclosure.

### DISCLOSURE OF THE INVENTION

### Problems to be Solved

The object of the present disclosure is, firstly, to provide a plurality of host materials which are able to produce an organic electroluminescent device with improved driving voltage and/or luminous efficiency and/or lifespan characteristics. Another object of the present disclosure is to provide an organic electroluminescent device with low driving voltage and/or high luminous efficiency and/or long lifespan characteristics by comprising a specific combination of compounds, or an organic electroluminescent compound according to the present disclosure.

### Solution to Problems

As a result of intensive study to solve the above-detailed technical problem, the present inventors found that the aforementioned objective can be achieved by a plurality of host materials comprising at least one first host compound represented by the following Formula 1, and at least one second host compound represented by the following Formula 2, so that the present invention was completed.

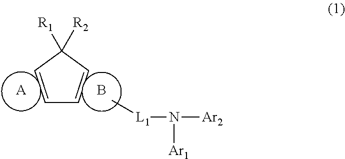

In Formula 1,
ring A and ring B each independently represent, a benzene ring or a naphthalene ring; provided that at least one of ring A and ring B is a naphthalene ring;
R₁ and R₂ each independently represent, hydrogen, deuterium, halogen, cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono-or di-(C1-C30)alkylamino, a substituted or unsubstituted mono-or di-(C6-C30)arylamino, or a substituted or unsubstituted (C1-C30)alkyl(C6-C30)arylamino; or may be linked to the adjacent substituents to form a ring(s);
L₁ represents a single bond, a substituted or unsubstituted (C6-C30)arylene, or a substituted or unsubstituted (3-30-membered)heteroarylene; and
Ar₁ and Ar₂ each independently represent, a substituted or unsubstituted (C6-C30)aryl, or a substituted or unsubstituted (3-to 30-membered)heteroaryl;

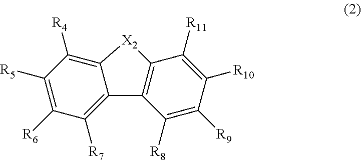

In Formula 2,
X₂ represents —O—, —S— or —Se—,
R₄ to R₆ and R₈ to R₁₁ each independently represent, hydrogen, deuterium, or a substituted or unsubstituted (C6-C30)aryl; provided that the (C6-C30)aryl does not include a substituted or unsubstituted fluorenyl; and
R₇ represents the following Formula 2-a;

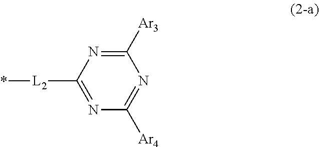

In Formula 2-a,
L2 represents a single bond, a substituted or unsubstituted (C6-C30)arylene, or a substituted or unsubstituted (3-to 30-membered)heteroarylene; and
Ar₃ and Ar₄ each independently represent, a substituted or unsubstituted (C6-C30)aryl, but do not include a substituted or unsubstituted fluorenyl.

### Advantageous Effects of Invention

By comprising a specific combination of compounds according to the present disclosure as host materials, an organic electroluminescent device having low driving voltage and/or high luminous efficiency and/or significantly improved long lifespan characteristics can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to a plurality of host materials comprising at least one first host compound represented by Formula 1 and at least one second host compound represented by Formula 2, and an organic electroluminescent device comprising the host materials.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. As such, at least two compounds may be comprised in the same layer or in different layers, and may be mixture-evaporated or co-evaporated, or may be individually evaporated.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C3-C30)cycloalkyl” is meant to be a mono-or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(3-to 7-membered)heterocycloalkyl” is meant to be a cycloalkyl having 3 to 7 ring backbone atoms and including at least one heteroatom selected from the group consisting of B, N, O, S, Si, and P, preferably the group consisting of O, S, and N, in which the number of the ring backbone carbon atoms is preferably 5 to 7, for example, tetrahydrofuran, pyrrolidine, thiolane, tetrahydropyran, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3-to 30-membered)heteroaryl(ene)” is an aryl having 3 to 30 ring backbone atoms and including at least one, preferably 1 to 4 heteroatom(s) selected from the group consisting of B, N, O, S, Si, P, Se, and Ge, in which the number of the ring backbone carbon atoms is preferably 3 to 30, and more preferably 5 to 20. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s), and may comprise a spiro structure. Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Herein, the term “a fused ring of (C3-C30) aliphatic ring and (C6-C30) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatom selected from B, N, O, S, Si, and P, preferably at least one heteroatom selected from N, O, and S. The term “Halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, the term “a ring formed in linking to an adjacent substituent” means a substituted or unsubstituted (3-to 30-membered) mono-or polycyclic, alicyclic, aromatic ring, or a combination thereof, formed by linking or fusing two or more adjacent substituents, preferably may be a substituted or unsubstituted (5-to 25-membered) mono-or polycyclic, alicyclic, aromatic ring, or a combination thereof. Further, the formed ring may include at least one heteroatom selected from the group consisting of B, N, O, S, Si, and P, preferably, N, O, and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be a heteroaryl or may be interpreted as one substituent in which two heteroaryls are connected. The substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted mono-or di-alkylamino, the substituted mono-or di-arylamino, and the substituted alkylarylamino in the formulas of the present disclosure, each independently may be substituted with at least one selected from the group consisting of deuterium; halogen; cyano; carboxyl; nitro; hydroxyl; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3-to 7-membered)heterocycloalkyl; (C6-C30)aryloxy; (C6-C30)arylthio; (3-to 30-membered)heteroaryl unsubstituted or substituted with at least one (C6-C30)aryl; (C6-C30)aryl unsubstituted or substituted with at least one of deuterium, halogen, cyano, (C1-C30)alkyl, (C6-C30)aryl, (3-to 30-membered)heteroaryl and tri(C6-C30)arylsilyl; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono-or di-(C1-C30)alkylamino; mono-or di-(C2-C30)alkenylamino; mono-or di-(C6-C30)arylamino; mono-or di-(3-30-membered)heteroarylamino; (C1-C30)alkyl(C2-C30)alkenylamino; (C1-C30)alkyl(C6-C30)arylamino; (C1-C30)alkyl(3-to 30-membered)heteroarylamino; (C2-C30)alkenyl(C6-C30)arylamino; (C2-C30)alkenyl(3-to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; di(C6-C30)arylboronyl; (C6-C30)arylphosphine; di(C1-C30)alkylboronyl; (C1-C30)alkyl(C6-C30)arylboronyl; (C6-C30)ar(C1-C30)alkyl; and (C1-C30)alkyl(C6-C30)aryl. For example, the substituents may be substituted with deuterium, methyl, phenylisopropyl, naphthyl unsubstituted or substituted with phenyl, biphenyl, fluorenyl substituted with phenyl, dimethylfluorenyl, triphenylenyl, pyrimidinyl substituted with phenyl, benzocarbazolyl substituted with phenyl, benzonaphthothiophenyl, triphenylsilyl, or diphenylamino, etc.

In the formulas of the present disclosure, when a plurality of substituents represented by the same symbol are present, each of these substituents, represented by the same symbol, may be the same or different.

Hereinafter, the plurality of host materials according to one embodiment will be described.

The plurality of host materials according to one embodiment comprise at least one first host compound represented by Formula 1 and at least one second host compound represented by Formula 2, and the plurality of host materials may be included in the light-emitting layer of an organic electroluminescent device according to one embodiment.

The first host compound as the host material according to one embodiment is represented by the following Formula 1.

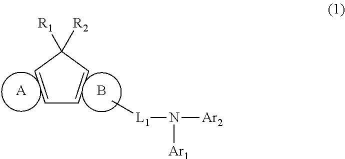

In Formula 1,
ring A and ring B each independently represent, a benzene ring or a naphthalene ring; provided that at least one of ring A and ring B is a naphthalene ring;
R₁ and R₂ each independently represent, hydrogen, deuterium, halogen, cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono-or di-(C1-C30)alkylamino, a substituted or unsubstituted mono-or di-(C6-C30)arylamino, or a substituted or unsubstituted (C1-C30)alkyl(C6-C30)arylamino; or may be linked to the adjacent substituents to form a ring(s);
L₁ represents a single bond, a substituted or unsubstituted (C6-C30)arylene, or a substituted or unsubstituted (3-to 30-membered)heteroarylene; and
Ar₁ and Ar₂ each independently represent, a substituted or unsubstituted (C6-C30)aryl or a substituted or unsubstituted (3-to 30-membered)heteroaryl.

In one embodiment, ring A may be a naphthalene ring, and ring B may be a benzene ring.

In one embodiment, ring A may be a benzene ring, and ring B may be a naphthalene ring.

According to one embodiment, Formula 1 may be represented by any one of the following Formulas 1-1 to 1-6.

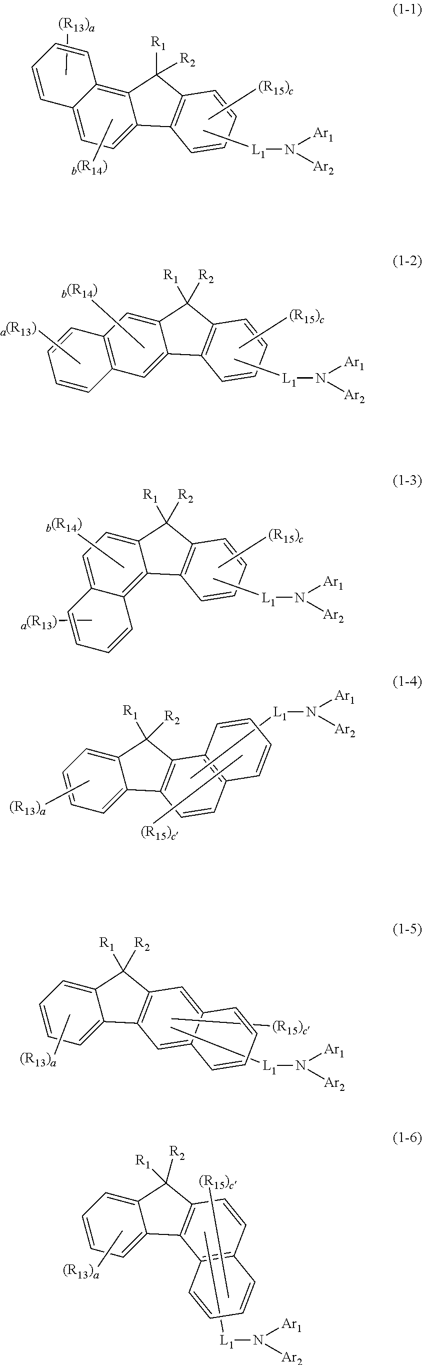

In Formulas 1-1 to 1-6,
R₁, R₂, L₁, Ar₁, and Ar₂ are as defined in Formula 1;
R₁₃ to R₁₅ are as defined as R₁ in Formula 1; and
a is an integer of 1 to 4; b is an integer of 1 or 2; c is an integer of 1 to 3; and c′ is an integer of 1 to 5; when a to c and c′ are an integer of 2 or more, each of R₁₃ to R₁₅ may be the same or different.

In one embodiment, R₁ and R₂ each independently may be a substituted or unsubstituted (C1-C30)alkyl, or a substituted or unsubstituted (C6-C30)aryl, or may be linked to each other to form a ring(s), preferably a substituted or unsubstituted (C1-C10) alkyl, or a substituted or unsubstituted (C6-C25)aryl, or may be linked to each other to form a substituted or unsubstituted (5-to 30-membered) monocyclic or polycyclic aromatic ring(s), more preferably a substituted or unsubstituted (C1-C4)alkyl, a substituted or unsubstituted (C6-C18)aryl, or may be linked to each other to form a substituted or unsubstituted (5-to 30-membered) polycyclic aromatic ring(s). For example, R₁ and R₂ each independently may be unsubstituted methyl, unsubstituted phenyl or tert-butyl substituted with deuterium; or may be linked to each other to form a fluorene ring.

In one embodiment, R₁₃ to R₁₅ each independently may be hydrogen, deuterium, a substituted or unsubstituted (C6-C30)aryl, a substituted or unsubstituted (5-to 30-membered)heteroaryl, or a substituted or unsubstituted di(C6-C30)arylamino, preferably hydrogen, deuterium, a substituted or unsubstituted (C6-C25)aryl, a substituted or unsubstituted (5-to 25-membered)heteroaryl, or a substituted or unsubstituted di(C6-C25)arylamino, more preferably hydrogen, deuterium, a substituted or unsubstituted (C6-C18)aryl, a substituted or unsubstituted (5-to 18-membered)heteroaryl, or a substituted or unsubstituted di(C6-C18)arylamino. For example, R₁₃ to R₁₅ each independently may be hydrogen, deuterium, a substituted or unsubstituted phenyl, a substituted or unsubstituted biphenyl, a substituted or unsubstituted diphenylamino, a substituted or unsubstituted phenylbiphenylamino, a substituted or unsubstituted pyridyl, or a substituted or unsubstituted dibenzothiophenyl.

In one embodiment, L₁ may be a single bond or a substituted or unsubstituted (C6-C30)arylene, preferably a single bond or a substituted or unsubstituted (C6-C25)arylene, more preferably a single bond or a substituted or unsubstituted (C6-C20)arylene. For example, L₁ may be a single bond, phenylene unsubstituted or substituted with diphenylamino, a substituted or unsubstituted naphthylenylene, a substituted or unsubstituted biphenylene, or a substituted or unsubstituted fluorenylene.

In one embodiment, Ar₁ and Ar₂ each independently may be a substituted or unsubstituted (C6-C30)aryl or a substituted or unsubstituted (5-to 30-membered)heteroaryl, preferably (C6-C25)aryl unsubstituted or substituted with at least one of deuterium; (C1-C10)alkyl; (C6-C30)aryl; (5-to 30-membered)heteroaryl; and di(C6-C30)arylamine, or (5-to 25-membered)heteroaryl unsubstituted or substituted with at least one of deuterium; (C1-C10)alkyl; (C6-C30)aryl; (5-to 30-membered)heteroaryl; and di(C6-C30)arylamine, more preferably (C6-C25)aryl unsubstituted or substituted with at least one of deuterium; (C1-C4)alkyl; (C6-C25)aryl; (5-to 25-membered)heteroaryl; and di(C6-C25)arylamine, or (5- to 25-membered)heteroaryl unsubstituted or substituted with at least one of deuterium; (C1-C4)alkyl; (C6-C25)aryl; (5-to 25-membered)heteroaryl; and di(C6-C25)arylamine. For example, Ar₁ and Ar₂ each independently may be phenyl unsubstituted or substituted with at least one of deuterium; methyl; phenylisopropyl; naphthyl; fluorenyl substituted with phenyl; dimethylfluorenyl; triphenylenyl; pyrimidinyl substituted with phenyl; benzocarbazolyl substituted with phenyl; benzonaphthothiophenyl; and diphenylamino, naphthyl unsubstituted or substituted with phenyl, p-biphenyl unsubstituted or substituted with at least one of phenylisopropyl; and benzimidazolyl substituted with phenyl, a substituted or unsubstituted m-biphenyl, a substituted or unsubstituted o-biphenyl, a substituted or unsubstituted o-terphenyl, a substituted or unsubstituted m-terphenyl, a substituted or unsubstituted p-terphenyl, triphenylenyl unsubstituted or substituted with phenyl, a substituted or unsubstituted o-tetraphenyl, a substituted or unsubstituted phenanthrenyl, fluorenyl unsubstituted or substituted with at least one of methyl and phenyl, benzofluorenyl unsubstituted or substituted with at least one of methyl and phenyl, spirobifluorenyl unsubstituted or substituted with benzofuranyl, carbazolyl unsubstituted or substituted with phenyl or biphenyl, dibenzofuranyl unsubstituted or substituted with phenyl, dibenzotihophenyl unsubstituted or substituted with phenyl or diphenylamino, or a substituted or unsubstituted dibenzoselenophenyl.

According to one embodiment, the first host compound represented by Formula 1 may be more specifically illustrated by the following compounds, but is not limited thereto.

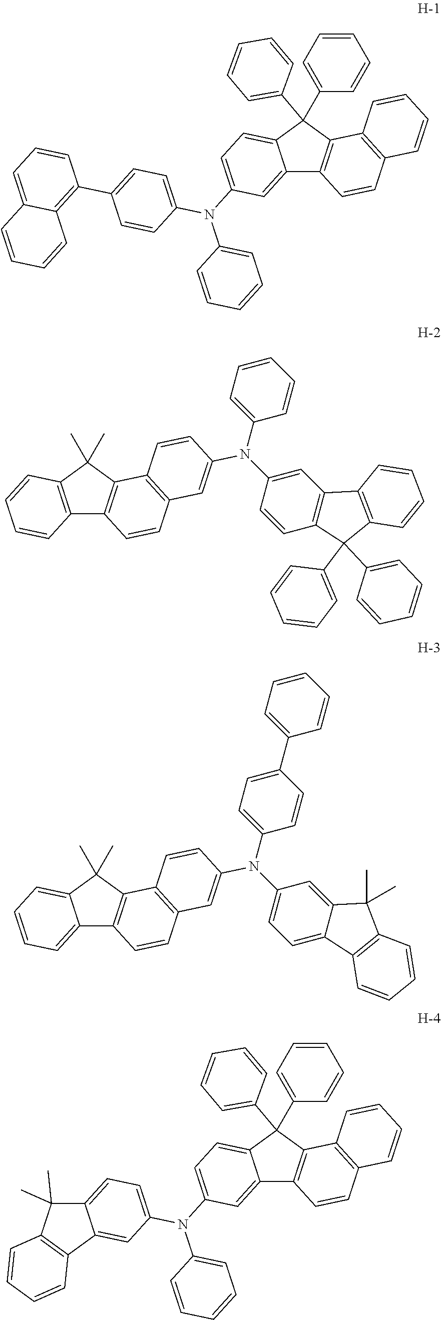 [SMILES: c1ccc(N(c2ccc(-c3cccc4ccccc34)cc2)c2ccc3c(c2)-c2ccc4ccccc4c2C3(c2ccccc2)c2ccccc2)cc1 ; CC1(C)c2ccccc2-c2ccc3cc(N(c4ccccc4)c4ccc5c(c4)-c4ccccc4C5(c4ccccc4)c4ccccc4)ccc3c21 ; CC1(C)c2ccccc2-c2ccc(N(c3ccc(-c4ccccc4)cc3)c3ccc4c5c(ccc4c3)-c3ccccc3C5(C)C)cc21 ; CC1(C)c2ccccc2-c2cc(N(c3ccccc3)c3ccc4c(c3)-c3ccc5ccccc5c3C4(c3ccccc3)c3ccccc3)ccc21]

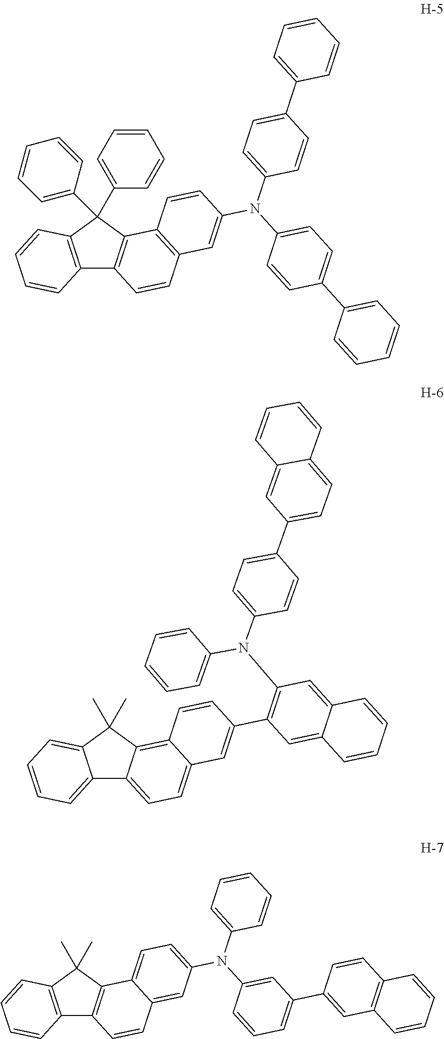 [SMILES: c1ccc(-c2ccc(N(c3ccc(-c4ccccc4)cc3)c3ccc4c5c(ccc4c3)-c3ccccc3C5(c3ccccc3)c3ccccc3)cc2)cc1 ; CC1(C)c2ccccc2-c2ccc3cc(-c4cc5ccccc5cc4N(c4ccccc4)c4ccc(-c5ccc6ccccc6c5)cc4)ccc3c21 ; CC1(C)c2ccccc2-c2ccc3cc(N(c4ccccc4)c4cccc(-c5ccc6ccccc6c5)c4)ccc3c21]

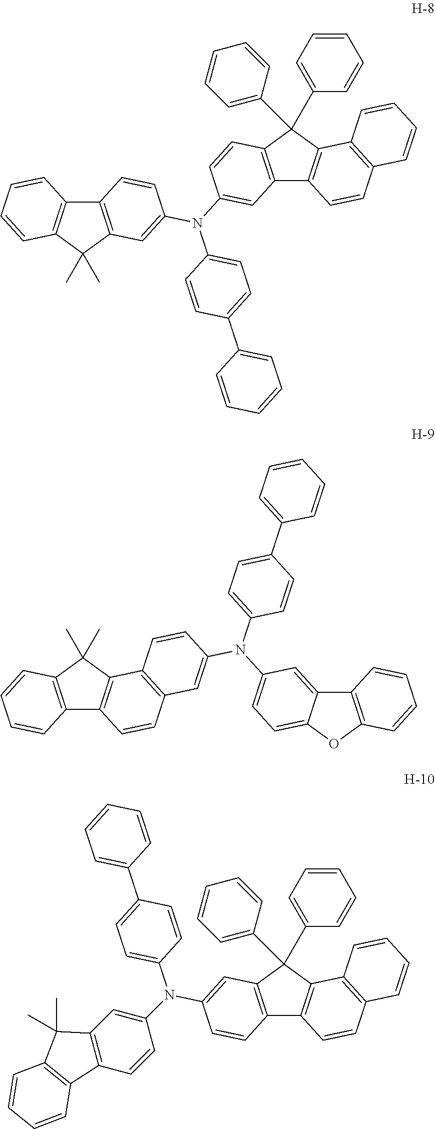 [SMILES: CC1(C)c2ccccc2-c2ccc(N(c3ccc(-c4ccccc4)cc3)c3ccc4c(c3)-c3ccc5ccccc5c3C4(c3ccccc3)c3ccccc3)cc21 ; CC1(C)c2ccccc2-c2ccc3cc(N(c4ccc(-c5ccccc5)cc4)c4ccc5oc6ccccc6c5c4)ccc3c21 ; CC1(C)c2ccccc2-c2ccc(N(c3ccc(-c4ccccc4)cc3)c3ccc4c(c3)C(c3ccccc3)(c3ccccc3)c3c-4ccc4ccccc34)cc21]

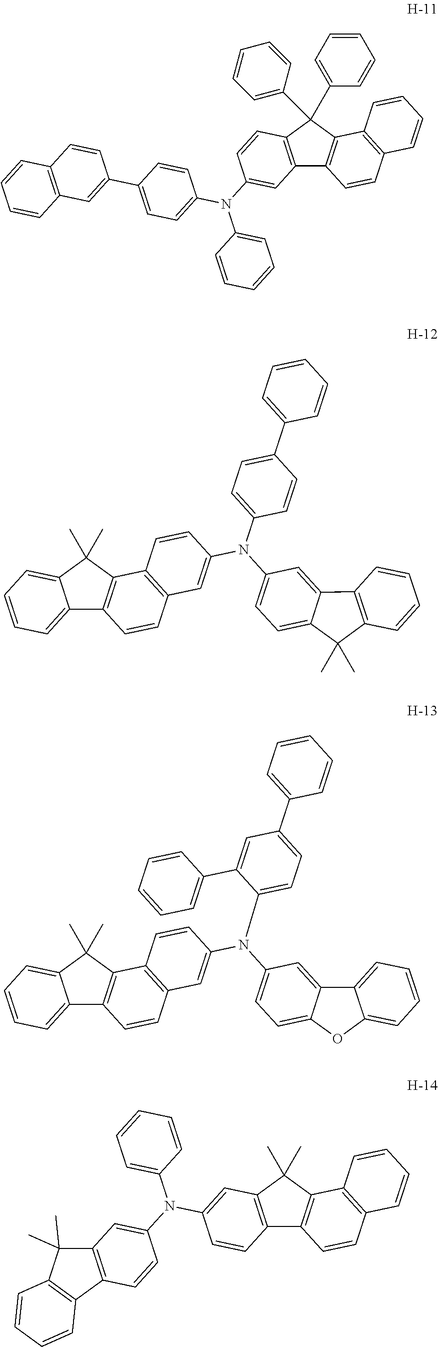 [SMILES: c1ccc(N(c2ccc(-c3ccc4ccccc4c3)cc2)c2ccc3c(c2)-c2ccc4ccccc4c2C3(c2ccccc2)c2ccccc2)cc1 ; CC1(C)c2ccccc2-c2cc(N(c3ccc(-c4ccccc4)cc3)c3ccc4c5c(ccc4c3)-c3ccccc3C5(C)C)ccc21 ; CC1(C)c2ccccc2-c2ccc3cc(N(c4ccc5oc6ccccc6c5c4)c4ccc(-c5ccccc5)cc4-c4ccccc4)ccc3c21 ; CC1(C)c2ccccc2-c2ccc(N(c3ccccc3)c3ccc4c(c3)C(C)(C)c3c-4ccc4ccccc34)cc21]

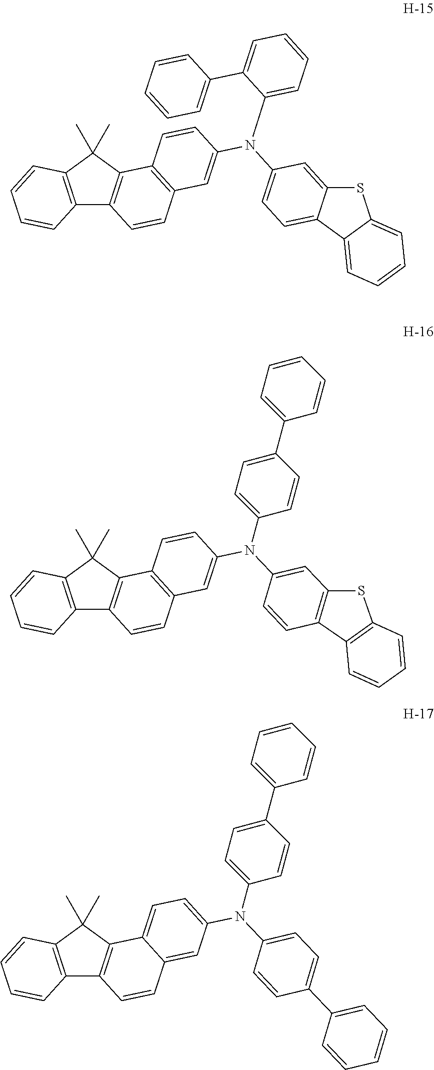 [SMILES: CC1(C)c2ccccc2-c2ccc3cc(N(c4ccc5c(c4)sc4ccccc45)c4ccccc4-c4ccccc4)ccc3c21 ; CC1(C)c2ccccc2-c2ccc3cc(N(c4ccc(-c5ccccc5)cc4)c4ccc5c(c4)sc4ccccc45)ccc3c21 ; CC1(C)c2ccccc2-c2ccc3cc(N(c4ccc(-c5ccccc5)cc4)c4ccc(-c5ccccc5)cc4)ccc3c21]

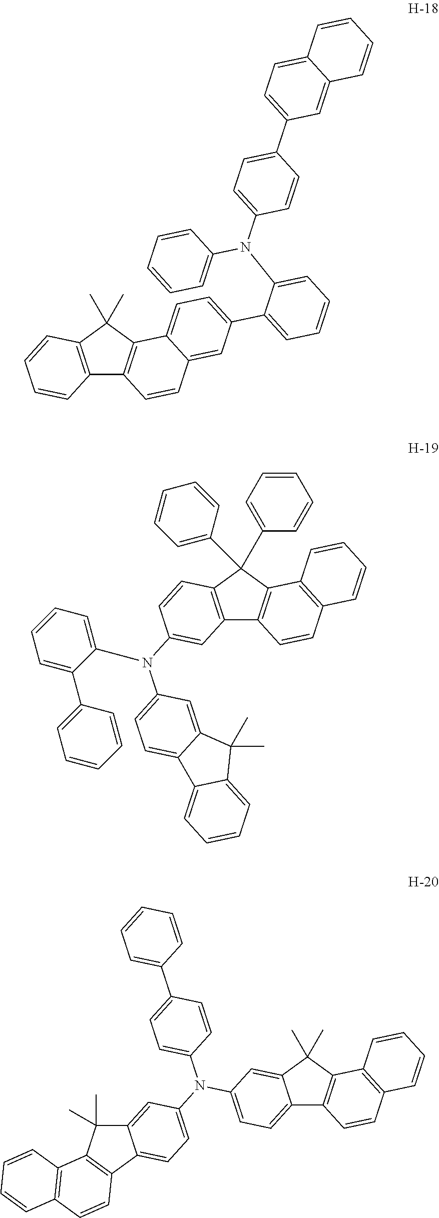 [SMILES: CC1(C)c2ccccc2-c2ccc3cc(-c4ccccc4N(c4ccccc4)c4ccc(-c5ccc6ccccc6c5)cc4)ccc3c21 ; CC1(C)c2ccccc2-c2ccc(N(c3ccc4c(c3)-c3ccc5ccccc5c3C4(c3ccccc3)c3ccccc3)c3ccccc3-c3ccccc3)cc21 ; CC1(C)c2cc(N(c3ccc(-c4ccccc4)cc3)c3ccc4c(c3)C(C)(C)c3c-4ccc4ccccc34)ccc2-c2ccc3ccccc3c21]

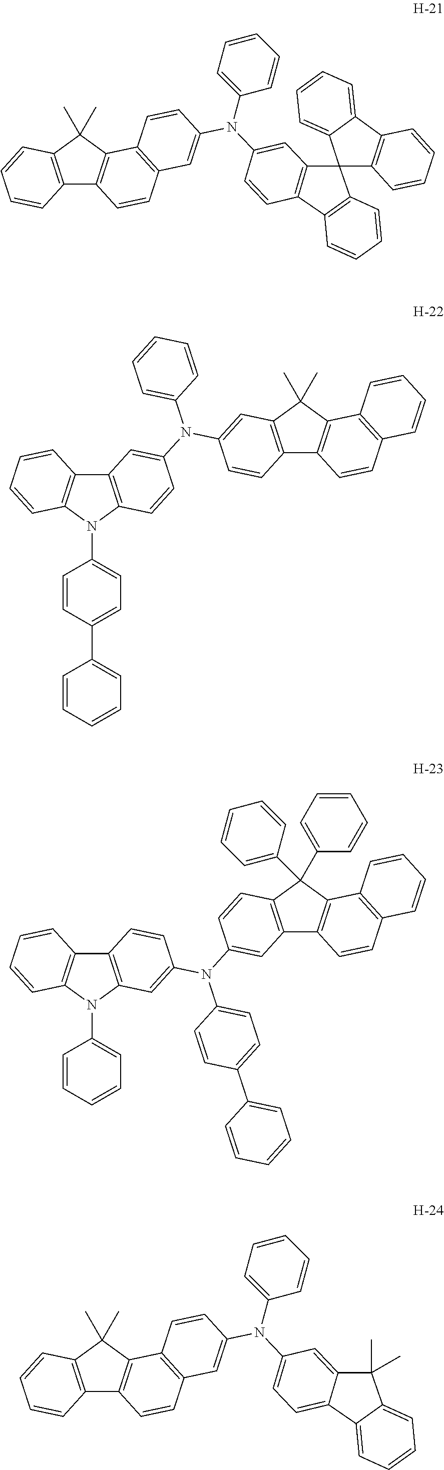 [SMILES: CC1(C)c2ccccc2-c2ccc3cc(N(c4ccccc4)c4ccc5c(c4)C4(c6ccccc6-c6ccccc64)c4ccccc4-5)ccc3c21 ; CC1(C)c2cc(N(c3ccccc3)c3ccc4c(c3)c3ccccc3n4-c3ccc(-c4ccccc4)cc3)ccc2-c2ccc3ccccc3c21 ; c1ccc(-c2ccc(N(c3ccc4c(c3)-c3ccc5ccccc5c3C4(c3ccccc3)c3ccccc3)c3ccc4c5ccccc5n(-c5ccccc5)c4c3)cc2)cc1 ; CC1(C)c2ccccc2-c2ccc(N(c3ccccc3)c3ccc4c5c(ccc4c3)-c3ccccc3C5(C)C)cc21]

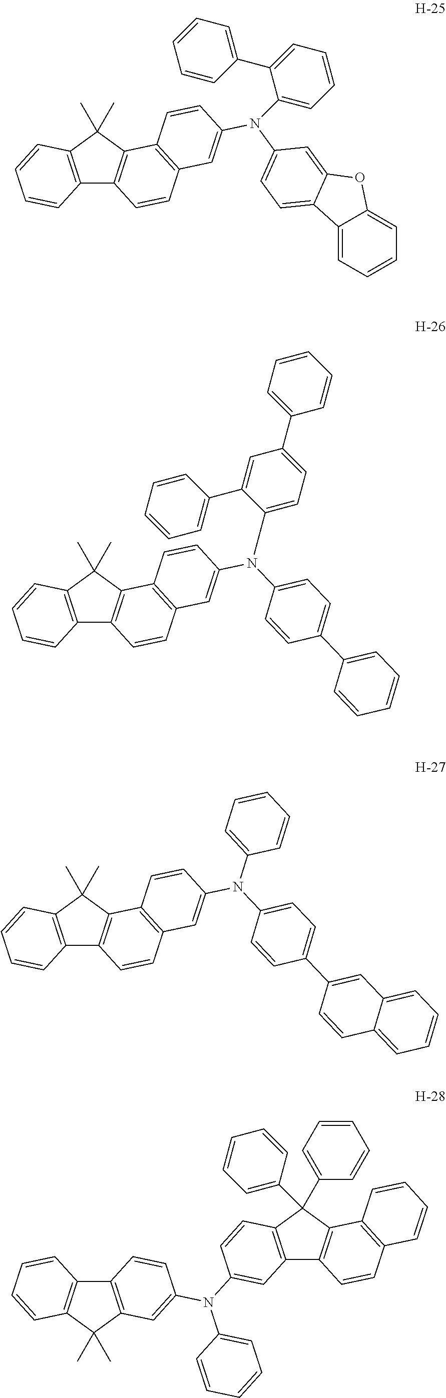 [SMILES: CC1(C)c2ccccc2-c2ccc3cc(N(c4ccc5c(c4)oc4ccccc45)c4ccccc4-c4ccccc4)ccc3c21 ; CC1(C)c2ccccc2-c2ccc3cc(N(c4ccc(-c5ccccc5)cc4)c4ccc(-c5ccccc5)cc4-c4ccccc4)ccc3c21 ; CC1(C)c2ccccc2-c2ccc3cc(N(c4ccccc4)c4ccc(-c5ccc6ccccc6c5)cc4)ccc3c21 ; CC1(C)c2ccccc2-c2ccc(N(c3ccccc3)c3ccc4c(c3)-c3ccc5ccccc5c3C4(c3ccccc3)c3ccccc3)cc21]

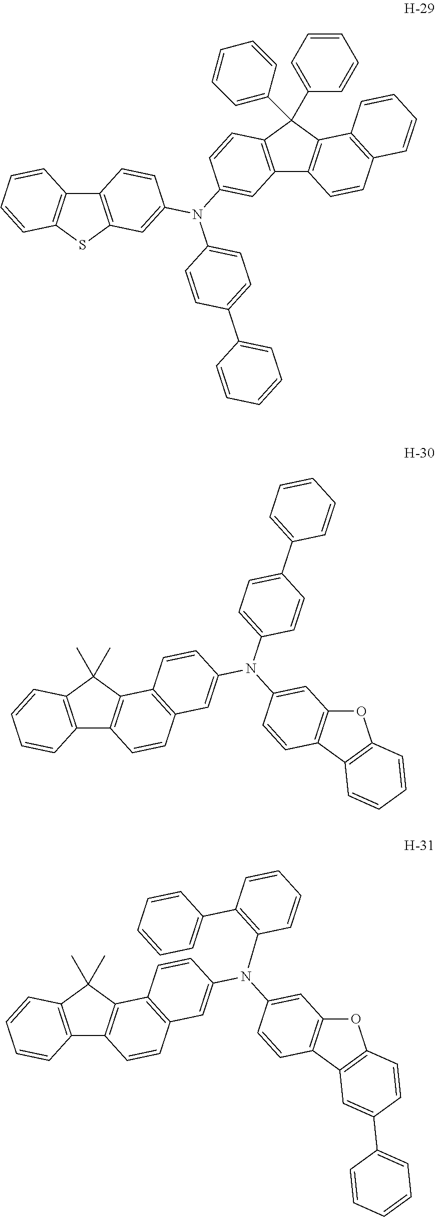 [SMILES: c1ccc(-c2ccc(N(c3ccc4c(c3)-c3ccc5ccccc5c3C4(c3ccccc3)c3ccccc3)c3ccc4c(c3)sc3ccccc34)cc2)cc1 ; CC1(C)c2ccccc2-c2ccc3cc(N(c4ccc(-c5ccccc5)cc4)c4ccc5c(c4)oc4ccccc45)ccc3c21 ; CC1(C)c2ccccc2-c2ccc3cc(N(c4ccc5c(c4)oc4ccc(-c6ccccc6)cc45)c4ccccc4-c4ccccc4)ccc3c21]

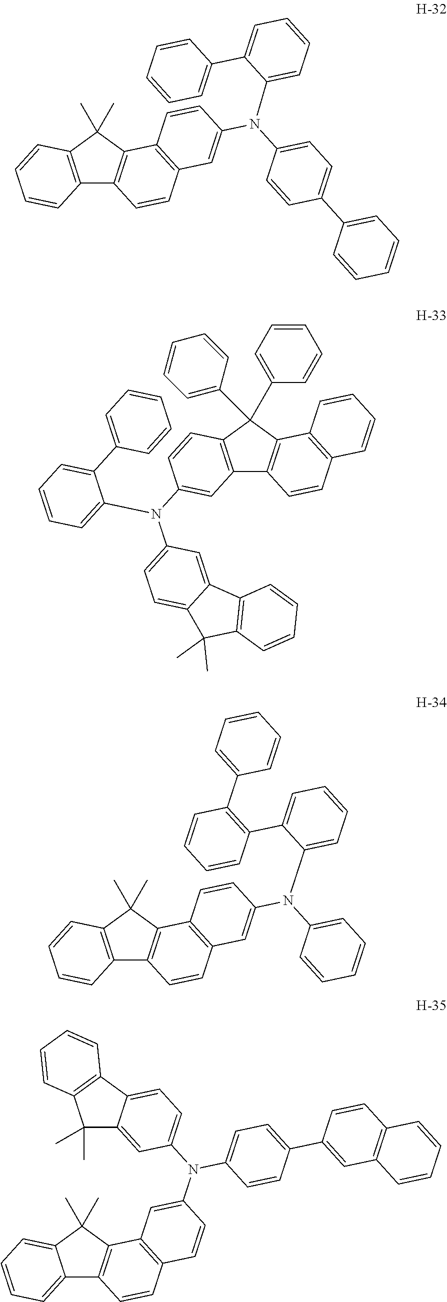 [SMILES: CC1(C)c2ccccc2-c2ccc3cc(N(c4ccc(-c5ccccc5)cc4)c4ccccc4-c4ccccc4)ccc3c21 ; CC1(C)c2ccccc2-c2cc(N(c3ccc4c(c3)-c3ccc5ccccc5c3C4(c3ccccc3)c3ccccc3)c3ccccc3-c3ccccc3)ccc21 ; CC1(C)c2ccccc2-c2ccc3cc(N(c4ccccc4)c4ccccc4-c4ccccc4-c4ccccc4)ccc3c21 ; CC1(C)c2ccccc2-c2ccc(N(c3ccc(-c4ccc5ccccc5c4)cc3)c3ccc4ccc5c(c4c3)C(C)(C)c3ccccc3-5)cc21]

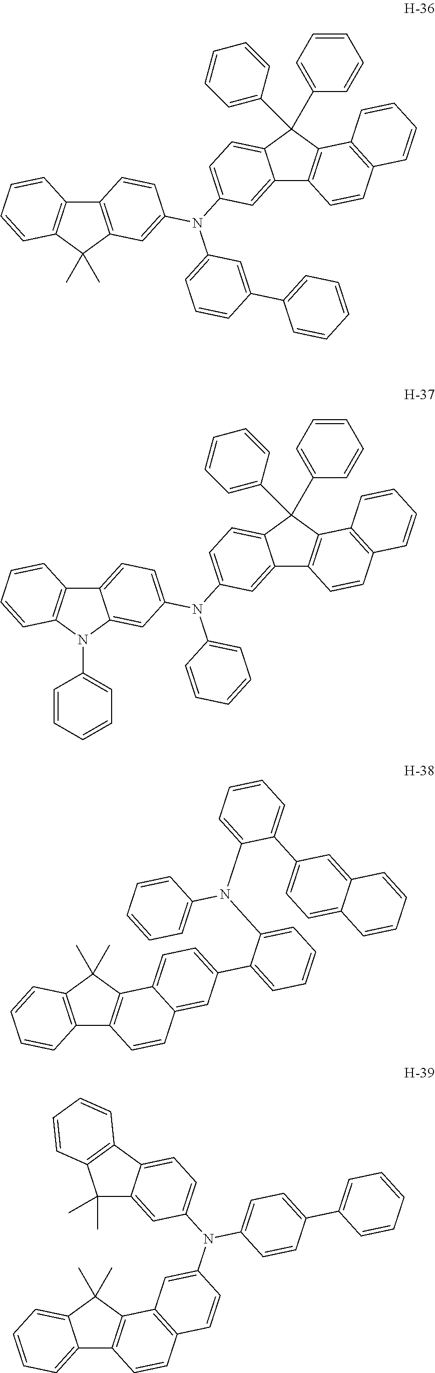 [SMILES: CC1(C)c2ccccc2-c2ccc(N(c3cccc(-c4ccccc4)c3)c3ccc4c(c3)-c3ccc5ccccc5c3C4(c3ccccc3)c3ccccc3)cc21 ; c1ccc(N(c2ccc3c(c2)-c2ccc4ccccc4c2C3(c2ccccc2)c2ccccc2)c2ccc3c4ccccc4n(-c4ccccc4)c3c2)cc1 ; CC1(C)c2ccccc2-c2ccc3cc(-c4ccccc4N(c4ccccc4)c4ccccc4-c4ccc5ccccc5c4)ccc3c21 ; CC1(C)c2ccccc2-c2ccc(N(c3ccc(-c4ccccc4)cc3)c3ccc4ccc5c(c4c3)C(C)(C)c3ccccc3-5)cc21]

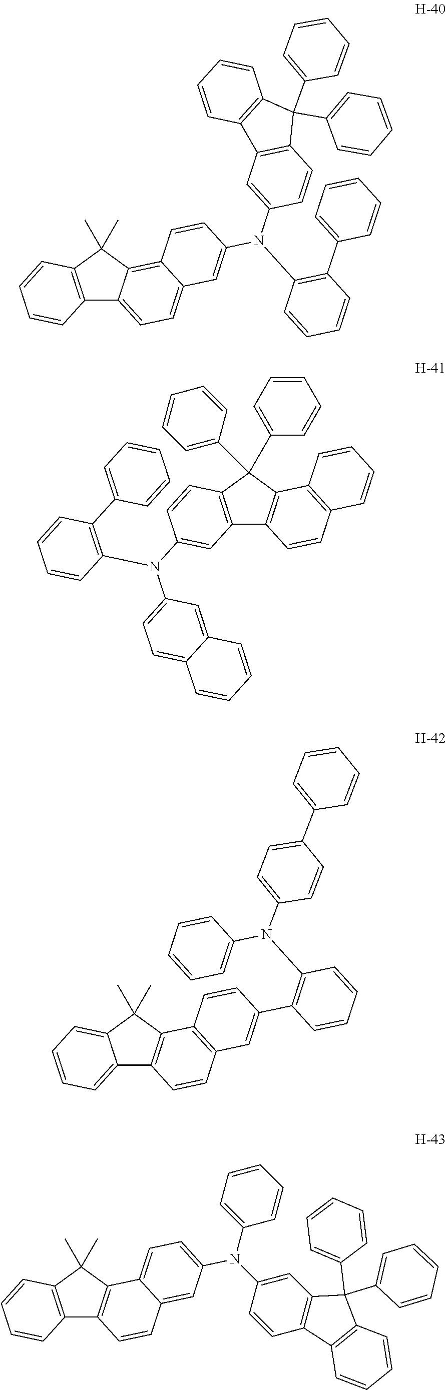

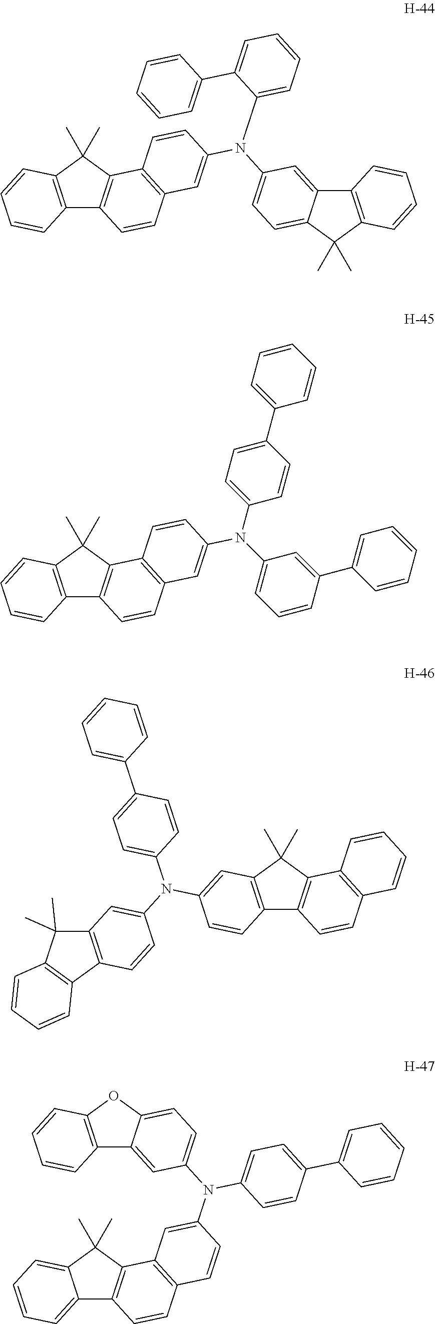 [SMILES: CC1(C)c2ccccc2-c2cc(N(c3ccc4c5c(ccc4c3)-c3ccccc3C5(C)C)c3ccccc3-c3ccccc3)ccc21 ; CC1(C)c2ccccc2-c2ccc3cc(N(c4ccc(-c5ccccc5)cc4)c4cccc(-c5ccccc5)c4)ccc3c21 ; CC1(C)c2ccccc2-c2ccc(N(c3ccc(-c4ccccc4)cc3)c3ccc4c(c3)C(C)(C)c3c-4ccc4ccccc34)cc21 ; CC1(C)c2ccccc2-c2ccc3ccc(N(c4ccc(-c5ccccc5)cc4)c4ccc5oc6ccccc6c5c4)cc3c21]

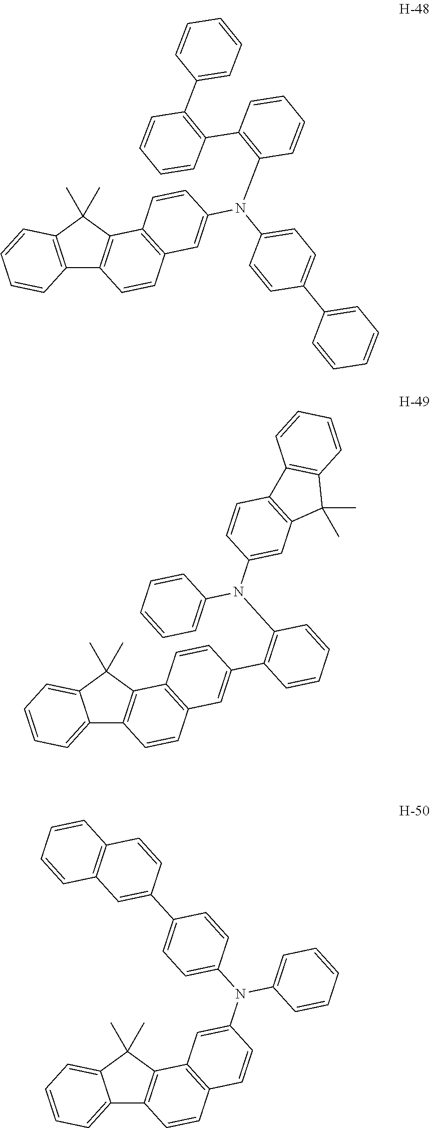 [SMILES: CC1(C)c2ccccc2-c2ccc3cc(N(c4ccc(-c5ccccc5)cc4)c4ccccc4-c4ccccc4-c4ccccc4)ccc3c21 ; CC1(C)c2ccccc2-c2ccc(N(c3ccccc3)c3ccccc3-c3ccc4c5c(ccc4c3)-c3ccccc3C5(C)C)cc21 ; CC1(C)c2ccccc2-c2ccc3ccc(N(c4ccccc4)c4ccc(-c5ccc6ccccc6c5)cc4)cc3c21]

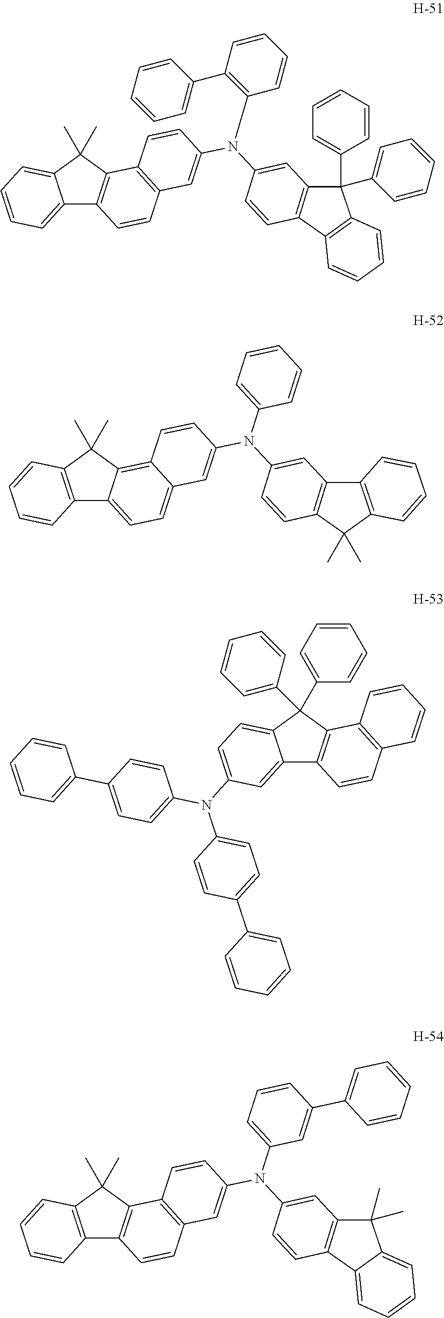 [SMILES: CC1(C)c2ccccc2-c2ccc3cc(N(c4ccc5c(c4)C(c4ccccc4)(c4ccccc4)c4ccccc4-5)c4ccccc4-c4ccccc4)ccc3c21 ; CC1(C)c2ccccc2-c2cc(N(c3ccccc3)c3ccc4c5c(ccc4c3)-c3ccccc3C5(C)C)ccc21 ; c1ccc(-c2ccc(N(c3ccc(-c4ccccc4)cc3)c3ccc4c(c3)-c3ccc5ccccc5c3C4(c3ccccc3)c3ccccc3)cc2)cc1 ; CC1(C)c2ccccc2-c2ccc(N(c3cccc(-c4ccccc4)c3)c3ccc4c5c(ccc4c3)-c3ccccc3C5(C)C)cc21]

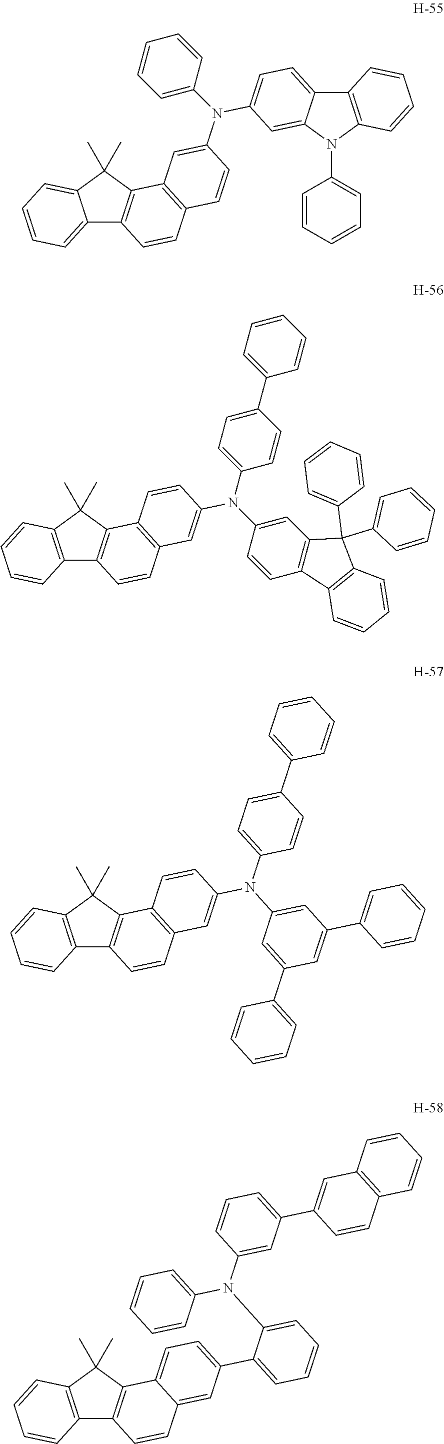 [SMILES: CC1(C)c2ccccc2-c2ccc3ccc(N(c4ccccc4)c4ccc5c6ccccc6n(-c6ccccc6)c5c4)cc3c21 ; CC1(C)c2ccccc2-c2ccc3cc(N(c4ccc(-c5ccccc5)cc4)c4ccc5c(c4)C(c4ccccc4)(c4ccccc4)c4ccccc4-5)ccc3c21 ; CC1(C)c2ccccc2-c2ccc3cc(N(c4ccc(-c5ccccc5)cc4)c4cc(-c5ccccc5)cc(-c5ccccc5)c4)ccc3c21 ; CC1(C)c2ccccc2-c2ccc3cc(-c4ccccc4N(c4ccccc4)c4cccc(-c5ccc6ccccc6c5)c4)ccc3c21]

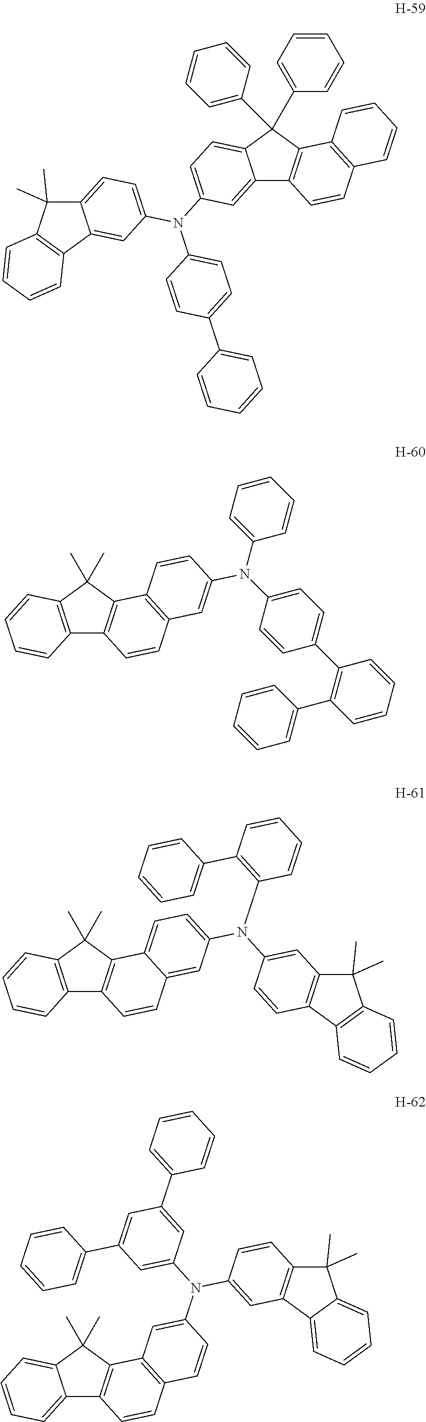 [SMILES: CC1(C)c2ccccc2-c2cc(N(c3ccc(-c4ccccc4)cc3)c3ccc4c(c3)-c3ccc5ccccc5c3C4(c3ccccc3)c3ccccc3)ccc21 ; CC1(C)c2ccccc2-c2ccc3cc(N(c4ccccc4)c4ccc(-c5ccccc5-c5ccccc5)cc4)ccc3c21 ; CC1(C)c2ccccc2-c2ccc(N(c3ccc4c5c(ccc4c3)-c3ccccc3C5(C)C)c3ccccc3-c3ccccc3)cc21 ; CC1(C)c2ccccc2-c2cc(N(c3cc(-c4ccccc4)cc(-c4ccccc4)c3)c3ccc4ccc5c(c4c3)C(C)(C)c3ccccc3-5)ccc21]

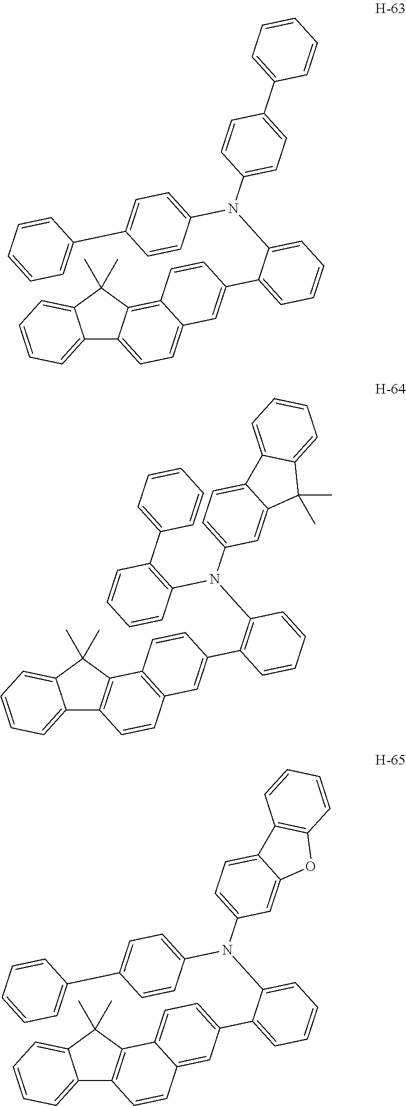 [SMILES: CC1(C)c2ccccc2-c2ccc3cc(-c4ccccc4N(c4ccc(-c5ccccc5)cc4)c4ccc(-c5ccccc5)cc4)ccc3c21 ; CC1(C)c2ccccc2-c2ccc(N(c3ccccc3-c3ccccc3)c3ccccc3-c3ccc4c5c(ccc4c3)-c3ccccc3C5(C)C)cc21 ; CC1(C)c2ccccc2-c2ccc3cc(-c4ccccc4N(c4ccc(-c5ccccc5)cc4)c4ccc5c(c4)oc4ccccc45)ccc3c21]

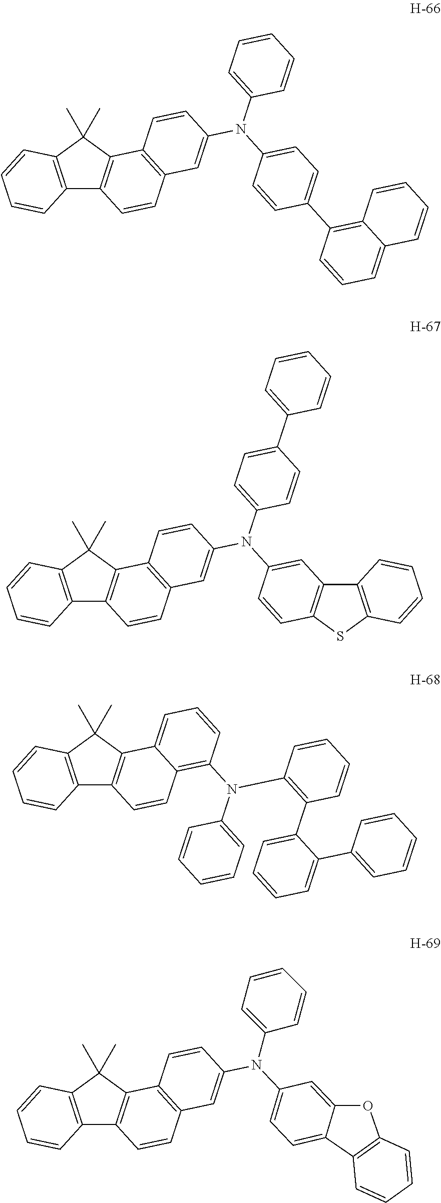 [SMILES: CC1(C)c2ccccc2-c2ccc3cc(N(c4ccccc4)c4ccc(-c5cccc6ccccc56)cc4)ccc3c21 ; CC1(C)c2ccccc2-c2ccc3cc(N(c4ccc(-c5ccccc5)cc4)c4ccc5sc6ccccc6c5c4)ccc3c21 ; CC1(C)c2ccccc2-c2ccc3c(N(c4ccccc4)c4ccccc4-c4ccccc4-c4ccccc4)cccc3c21 ; CC1(C)c2ccccc2-c2ccc3cc(N(c4ccccc4)c4ccc5c(c4)oc4ccccc45)ccc3c21]

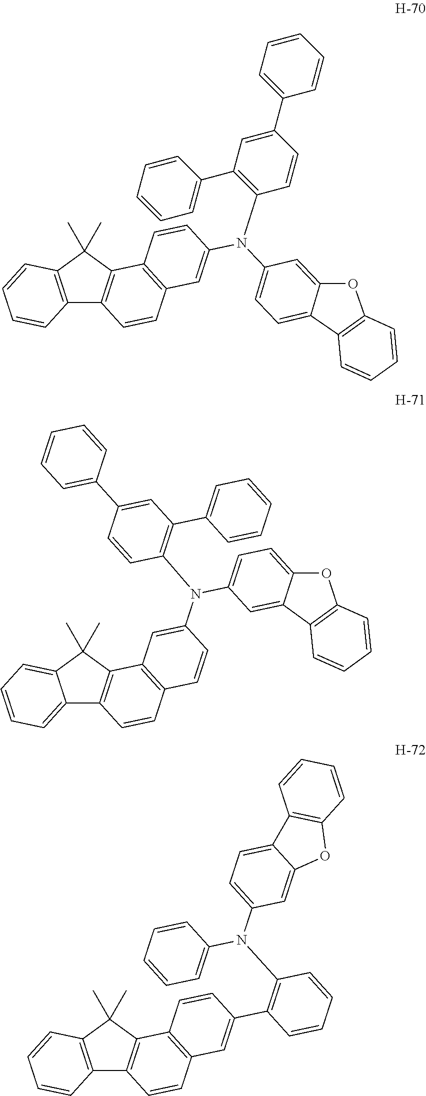 [SMILES: CC1(C)c2ccccc2-c2ccc3cc(N(c4ccc5c(c4)oc4ccccc45)c4ccc(-c5ccccc5)cc4-c4ccccc4)ccc3c21 ; CC1(C)c2ccccc2-c2ccc3ccc(N(c4ccc5oc6ccccc6c5c4)c4ccc(-c5ccccc5)cc4-c4ccccc4)cc3c21 ; CC1(C)c2ccccc2-c2ccc3cc(-c4ccccc4N(c4ccccc4)c4ccc5c(c4)oc4ccccc45)ccc3c21]

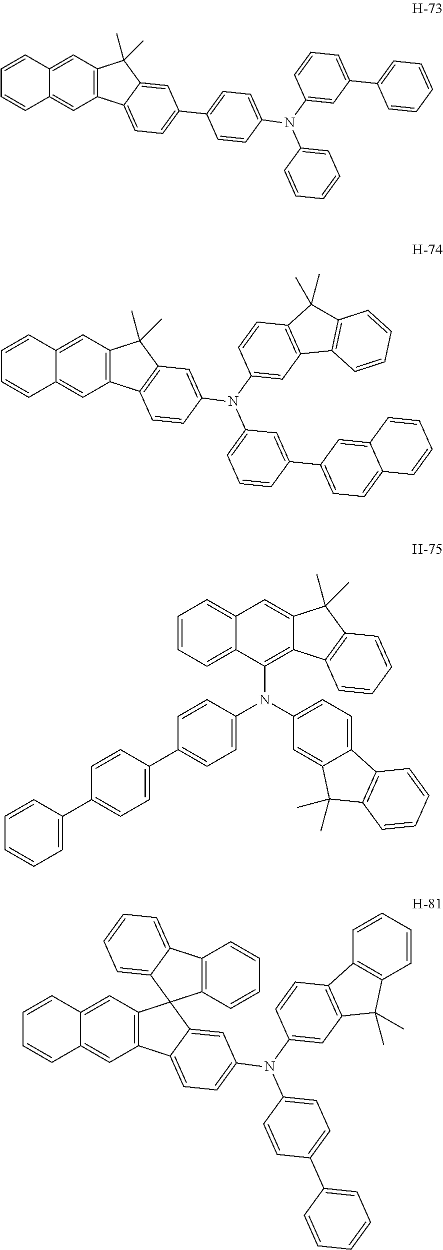 [SMILES: CC1(C)c2cc(-c3ccc(N(c4ccccc4)c4cccc(-c5ccccc5)c4)cc3)ccc2-c2cc3ccccc3cc21 ; CC1(C)c2ccccc2-c2cc(N(c3cccc(-c4ccc5ccccc5c4)c3)c3ccc4c(c3)C(C)(C)c3cc5ccccc5cc3-4)ccc21 ; CC1(C)c2ccccc2-c2ccc(N(c3ccc(-c4ccc(-c5ccccc5)cc4)cc3)c3c4c(cc5ccccc35)C(C)(C)c3ccccc3-4)cc21 ; CC1(C)c2ccccc2-c2ccc(N(c3ccc(-c4ccccc4)cc3)c3ccc4c(c3)C3(c5ccccc5-c5ccccc53)c3cc5ccccc5cc3-4)cc21]

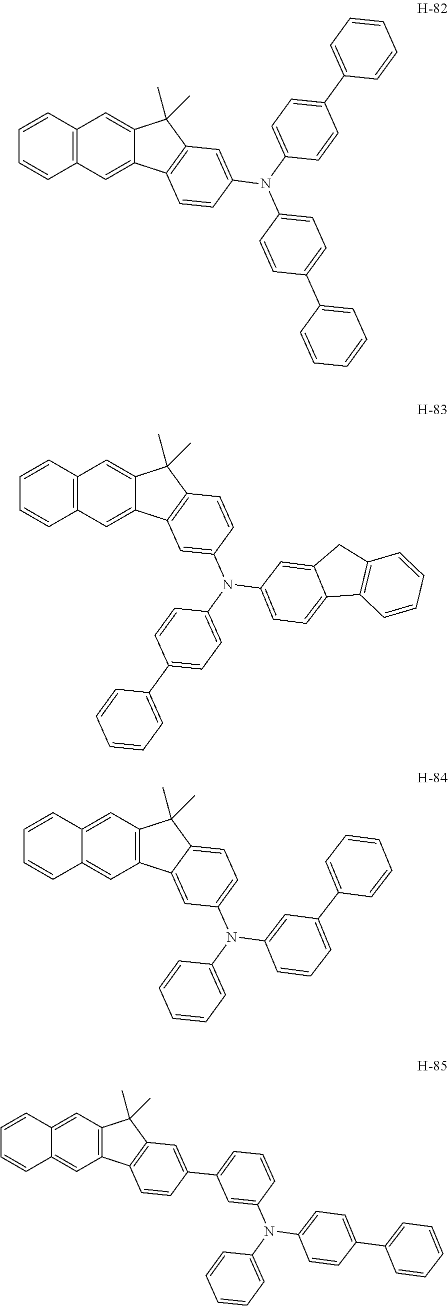

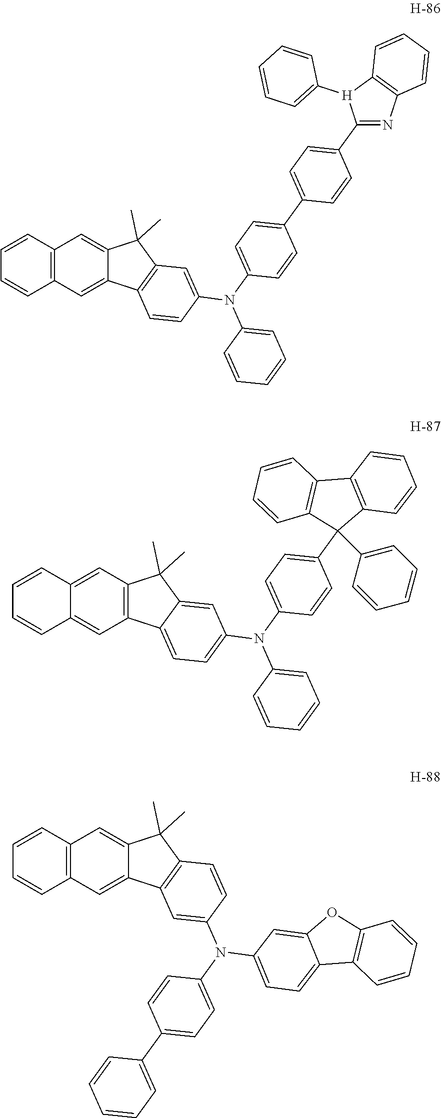

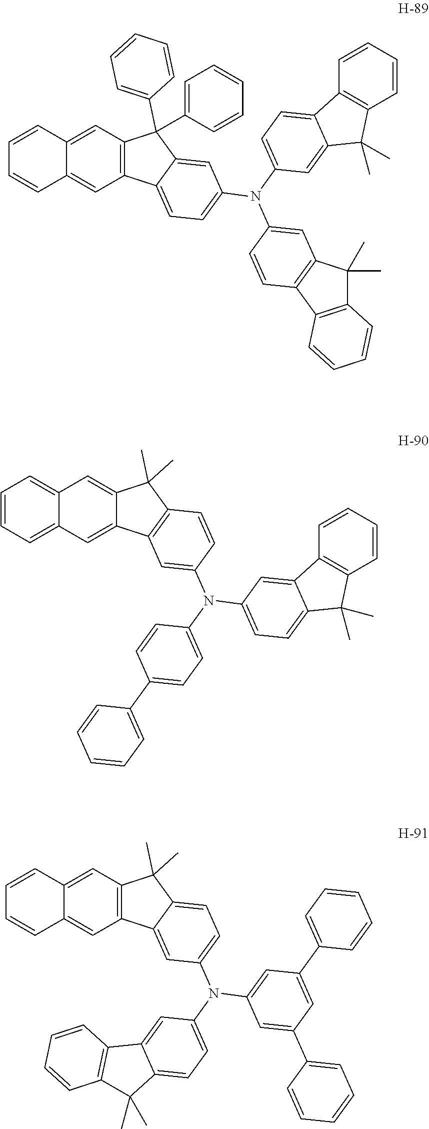 [SMILES: CC1(C)c2ccccc2-c2ccc(N(c3ccc4c(c3)C(C)(C)c3ccccc3-4)c3ccc4c(c3)C(c3ccccc3)(c3ccccc3)c3cc5ccccc5cc3-4)cc21 ; CC1(C)c2ccccc2-c2cc(N(c3ccc(-c4ccccc4)cc3)c3ccc4c(c3)-c3cc5ccccc5cc3C4(C)C)ccc21 ; CC1(C)c2ccccc2-c2cc(N(c3cc(-c4ccccc4)cc(-c4ccccc4)c3)c3ccc4c(c3)-c3cc5ccccc5cc3C4(C)C)ccc21]

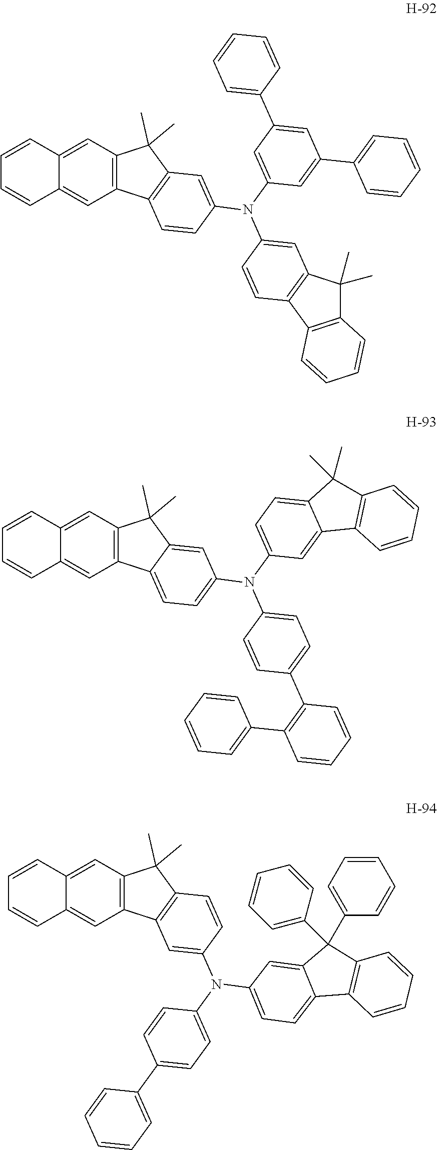 [SMILES: CC1(C)c2ccccc2-c2ccc(N(c3cc(-c4ccccc4)cc(-c4ccccc4)c3)c3ccc4c(c3)C(C)(C)c3cc5ccccc5cc3-4)cc21 ; CC1(C)c2ccccc2-c2cc(N(c3ccc(-c4ccccc4-c4ccccc4)cc3)c3ccc4c(c3)C(C)(C)c3cc5ccccc5cc3-4)ccc21 ; CC1(C)c2ccc(N(c3ccc(-c4ccccc4)cc3)c3ccc4c(c3)C(c3ccccc3)(c3ccccc3)c3ccccc3-4)cc2-c2cc3ccccc3cc21]

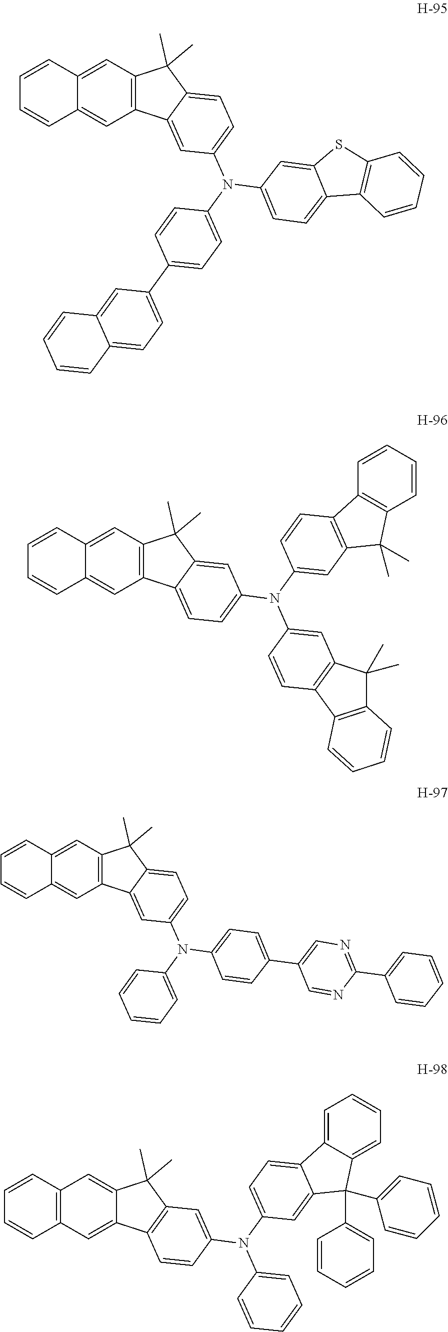 [SMILES: CC1(C)c2ccc(N(c3ccc(-c4ccc5ccccc5c4)cc3)c3ccc4c(c3)sc3ccccc34)cc2-c2cc3ccccc3cc21 ; CC1(C)c2ccccc2-c2ccc(N(c3ccc4c(c3)C(C)(C)c3ccccc3-4)c3ccc4c(c3)C(C)(C)c3cc5ccccc5cc3-4)cc21 ; CC1(C)c2ccc(N(c3ccccc3)c3ccc(-c4cnc(-c5ccccc5)nc4)cc3)cc2-c2cc3ccccc3cc21 ; CC1(C)c2cc(N(c3ccccc3)c3ccc4c(c3)C(c3ccccc3)(c3ccccc3)c3ccccc3-4)ccc2-c2cc3ccccc3cc21]

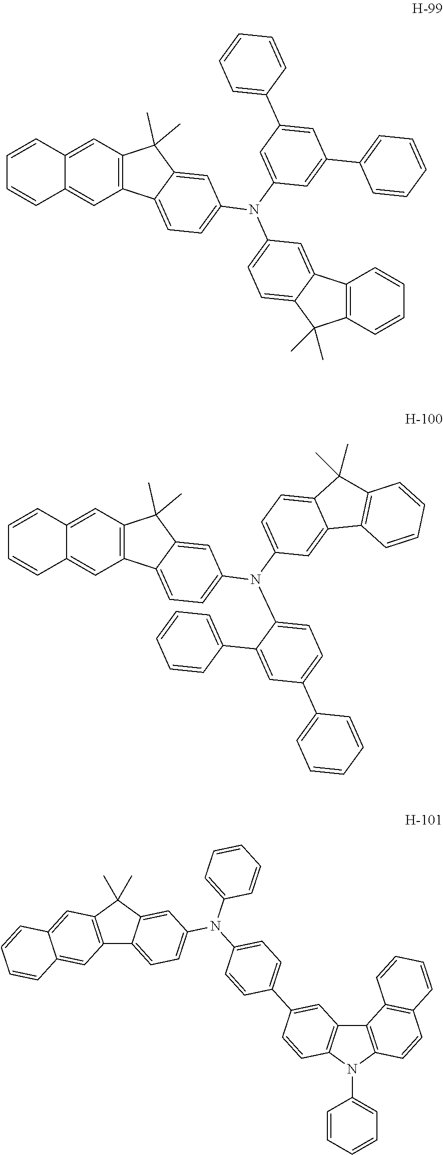 [SMILES: CC1(C)c2ccccc2-c2cc(N(c3cc(-c4ccccc4)cc(-c4ccccc4)c3)c3ccc4c(c3)C(C)(C)c3cc5ccccc5cc3-4)ccc21 ; CC1(C)c2ccccc2-c2cc(N(c3ccc4c(c3)C(C)(C)c3cc5ccccc5cc3-4)c3ccc(-c4ccccc4)cc3-c3ccccc3)ccc21 ; CC1(C)c2cc(N(c3ccccc3)c3ccc(-c4ccc5c(c4)c4c6ccccc6ccc4n5-c4ccccc4)cc3)ccc2-c2cc3ccccc3cc21]

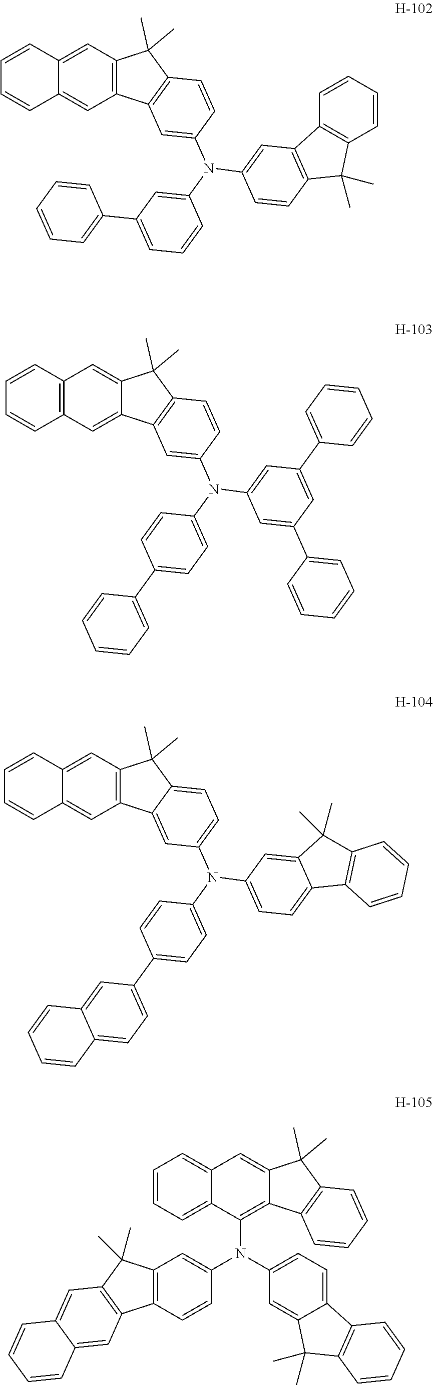 [SMILES: CC1(C)c2ccccc2-c2cc(N(c3cccc(-c4ccccc4)c3)c3ccc4c(c3)-c3cc5ccccc5cc3C4(C)C)ccc21 ; CC1(C)c2ccc(N(c3ccc(-c4ccccc4)cc3)c3cc(-c4ccccc4)cc(-c4ccccc4)c3)cc2-c2cc3ccccc3cc21 ; CC1(C)c2ccccc2-c2ccc(N(c3ccc(-c4ccc5ccccc5c4)cc3)c3ccc4c(c3)-c3cc5ccccc5cc3C4(C)C)cc21 ; CC1(C)c2ccccc2-c2ccc(N(c3ccc4c(c3)C(C)(C)c3cc5ccccc5cc3-4)c3c4c(cc5ccccc35)C(C)(C)c3ccccc3-4)cc21]

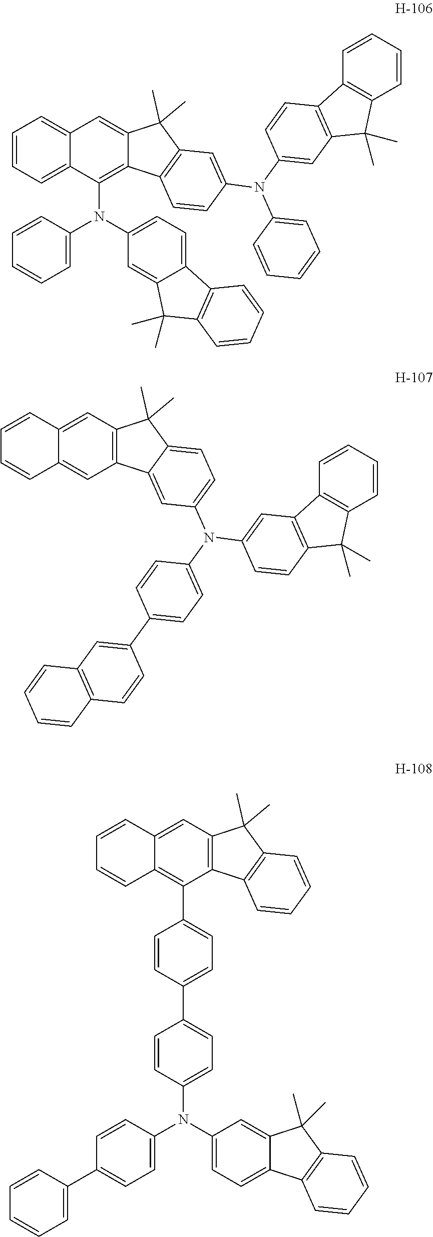

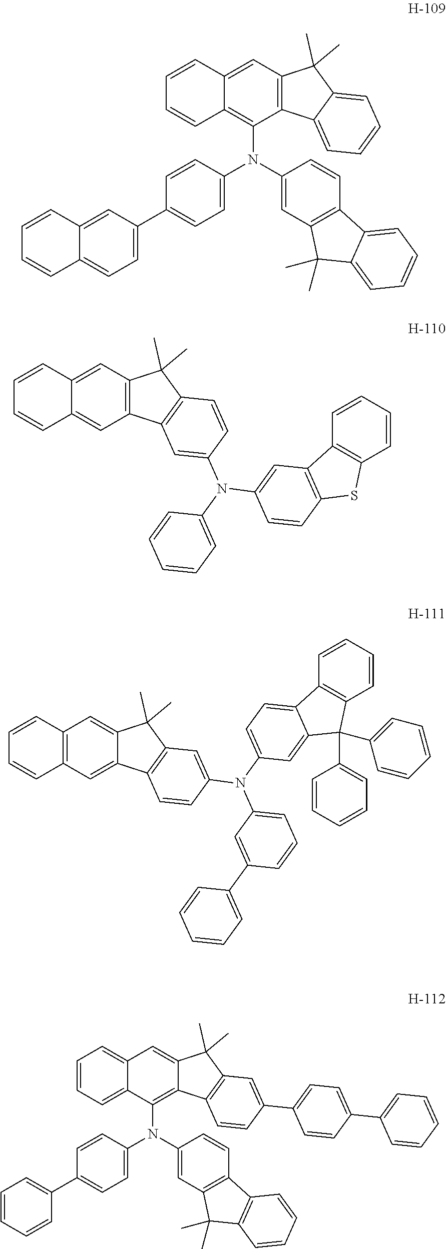 [SMILES: CC1(C)c2ccccc2-c2ccc(N(c3ccc(-c4ccc5ccccc5c4)cc3)c3c4c(cc5ccccc35)C(C)(C)c3ccccc3-4)cc21 ; CC1(C)c2ccc(N(c3ccccc3)c3ccc4sc5ccccc5c4c3)cc2-c2cc3ccccc3cc21 ; CC1(C)c2cc(N(c3cccc(-c4ccccc4)c3)c3ccc4c(c3)C(c3ccccc3)(c3ccccc3)c3ccccc3-4)ccc2-c2cc3ccccc3cc21 ; CC1(C)c2ccccc2-c2ccc(N(c3ccc(-c4ccccc4)cc3)c3c4c(cc5ccccc35)C(C)(C)c3cc(-c5ccc(-c6ccccc6)cc5)ccc3-4)cc21]

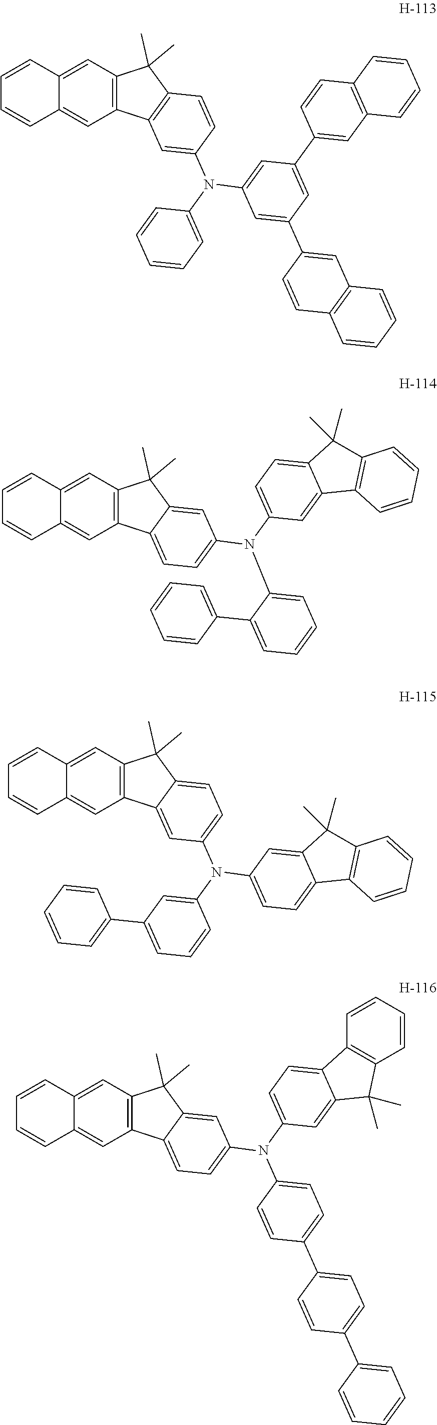 [SMILES: CC1(C)c2ccc(N(c3ccccc3)c3cc(-c4ccc5ccccc5c4)cc(-c4ccc5ccccc5c4)c3)cc2-c2cc3ccccc3cc21 ; CC1(C)c2ccccc2-c2cc(N(c3ccc4c(c3)C(C)(C)c3cc5ccccc5cc3-4)c3ccccc3-c3ccccc3)ccc21 ; CC1(C)c2ccccc2-c2ccc(N(c3cccc(-c4ccccc4)c3)c3ccc4c(c3)-c3cc5ccccc5cc3C4(C)C)cc21 ; CC1(C)c2ccccc2-c2ccc(N(c3ccc(-c4ccc(-c5ccccc5)cc4)cc3)c3ccc4c(c3)C(C)(C)c3cc5ccccc5cc3-4)cc21]

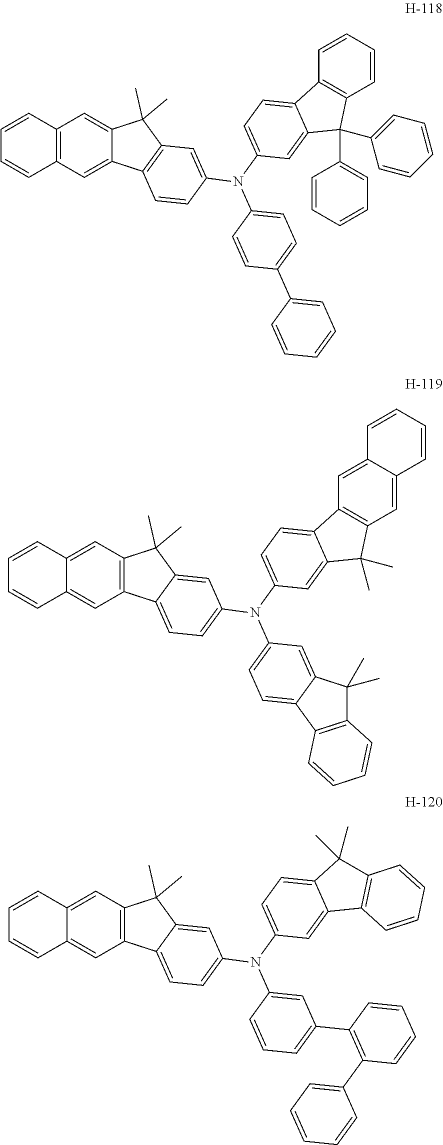

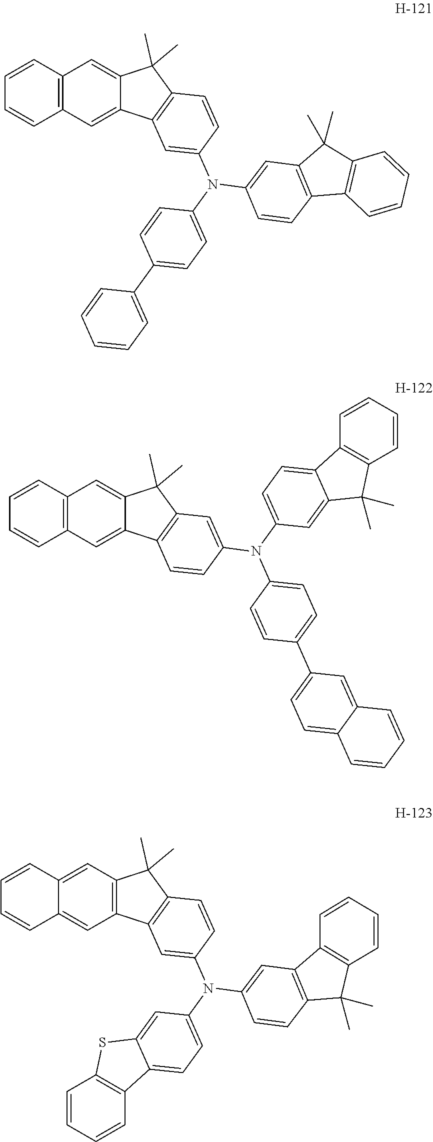 [SMILES: CC1(C)c2ccccc2-c2ccc(N(c3ccc(-c4ccccc4)cc3)c3ccc4c(c3)-c3cc5ccccc5cc3C4(C)C)cc21 ; CC1(C)c2ccccc2-c2ccc(N(c3ccc(-c4ccc5ccccc5c4)cc3)c3ccc4c(c3)C(C)(C)c3cc5ccccc5cc3-4)cc21 ; CC1(C)c2ccccc2-c2cc(N(c3ccc4c(c3)-c3cc5ccccc5cc3C4(C)C)c3ccc4c(c3)sc3ccccc34)ccc21]

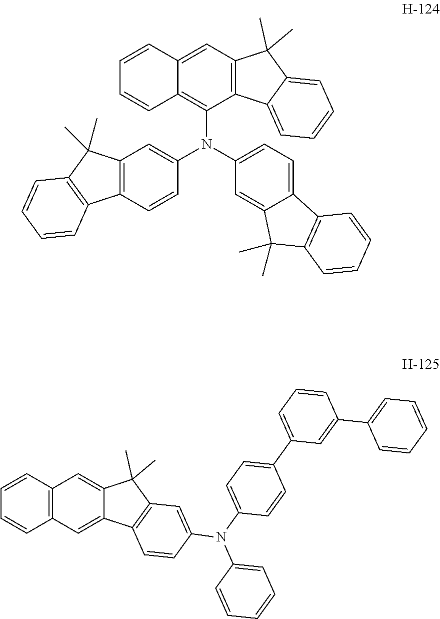 [SMILES: CC1(C)c2ccccc2-c2ccc(N(c3ccc4c(c3)C(C)(C)c3ccccc3-4)c3c4c(cc5ccccc35)C(C)(C)c3ccccc3-4)cc21 ; CC1(C)c2cc(N(c3ccccc3)c3ccc(-c4cccc(-c5ccccc5)c4)cc3)ccc2-c2cc3ccccc3cc21]

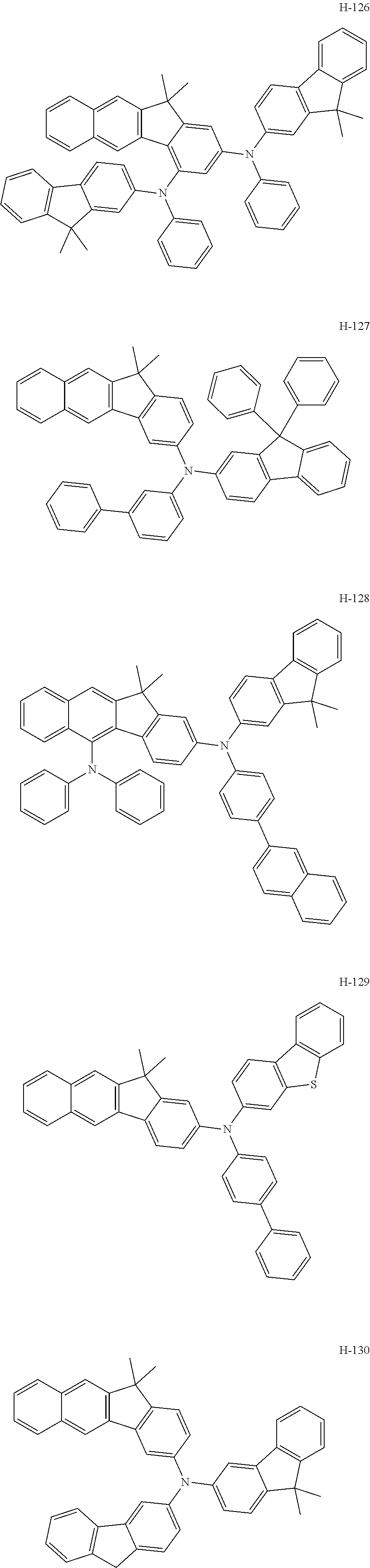 [SMILES: CC1(C)c2ccccc2-c2ccc(N(c3ccccc3)c3cc(N(c4ccccc4)c4ccc5c(c4)C(C)(C)c4ccccc4-5)c4c(c3)C(C)(C)c3cc5ccccc5cc3-4)cc21 ; CC1(C)c2ccc(N(c3cccc(-c4ccccc4)c3)c3ccc4c(c3)C(c3ccccc3)(c3ccccc3)c3ccccc3-4)cc2-c2cc3ccccc3cc21 ; CC1(C)c2ccccc2-c2ccc(N(c3ccc(-c4ccc5ccccc5c4)cc3)c3ccc4c(c3)C(C)(C)c3cc5ccccc5c(N(c5ccccc5)c5ccccc5)c3-4)cc21 ; CC1(C)c2cc(N(c3ccc(-c4ccccc4)cc3)c3ccc4c(c3)sc3ccccc34)ccc2-c2cc3ccccc3cc21 ; CC1(C)c2ccccc2-c2cc(N(c3ccc4c(c3)-c3ccccc3C4)c3ccc4c(c3)-c3cc5ccccc5cc3C4(C)C)ccc21]

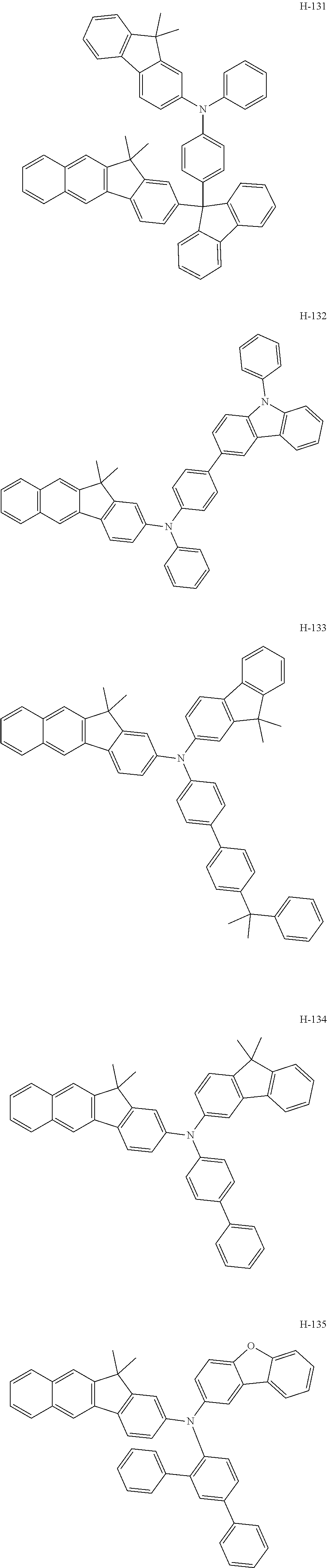 [SMILES: CC1(C)c2ccccc2-c2ccc(N(c3ccccc3)c3ccc(C4(c5ccc6c(c5)C(C)(C)c5cc7ccccc7cc5-6)c5ccccc5-c5ccccc54)cc3)cc21 ; CC1(C)c2cc(N(c3ccccc3)c3ccc(-c4ccc5c(c4)c4ccccc4n5-c4ccccc4)cc3)ccc2-c2cc3ccccc3cc21 ; CC(C)(c1ccccc1)c1ccc(-c2ccc(N(c3ccc4c(c3)C(C)(C)c3ccccc3-4)c3ccc4c(c3)C(C)(C)c3cc5ccccc5cc3-4)cc2)cc1 ; CC1(C)c2ccccc2-c2cc(N(c3ccc(-c4ccccc4)cc3)c3ccc4c(c3)C(C)(C)c3cc5ccccc5cc3-4)ccc21 ; CC1(C)c2cc(N(c3ccc4oc5ccccc5c4c3)c3ccc(-c4ccccc4)cc3-c3ccccc3)ccc2-c2cc3ccccc3cc21]

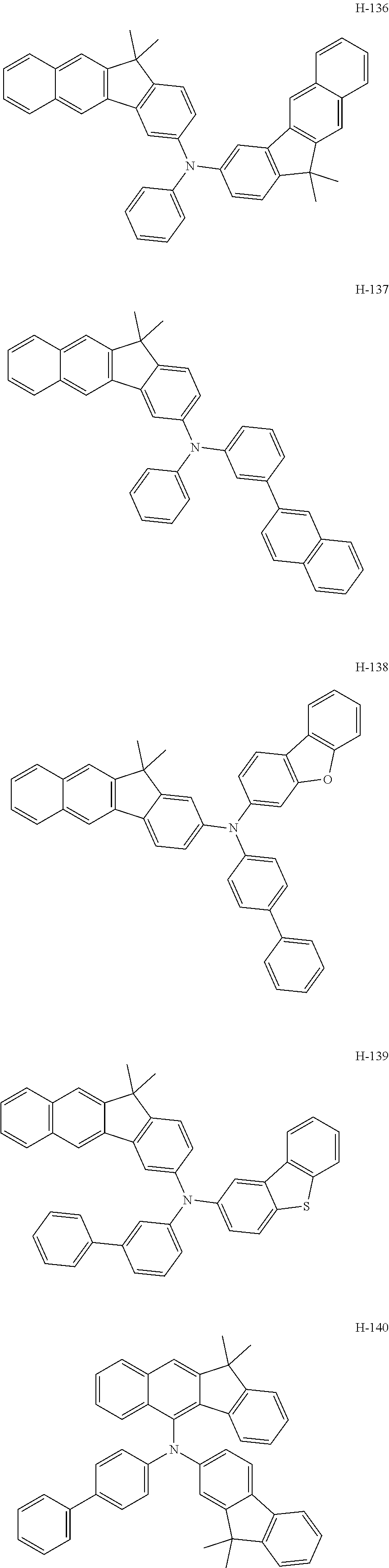 [SMILES: CC1(C)c2ccc(N(c3ccccc3)c3ccc4c(c3)-c3cc5ccccc5cc3C4(C)C)cc2-c2cc3ccccc3cc21 ; CC1(C)c2ccc(N(c3ccccc3)c3cccc(-c4ccc5ccccc5c4)c3)cc2-c2cc3ccccc3cc21 ; CC1(C)c2cc(N(c3ccc(-c4ccccc4)cc3)c3ccc4c(c3)oc3ccccc34)ccc2-c2cc3ccccc3cc21 ; CC1(C)c2ccc(N(c3cccc(-c4ccccc4)c3)c3ccc4sc5ccccc5c4c3)cc2-c2cc3ccccc3cc21 ; CC1(C)c2ccccc2-c2ccc(N(c3ccc(-c4ccccc4)cc3)c3c4c(cc5ccccc35)C(C)(C)c3ccccc3-4)cc21]

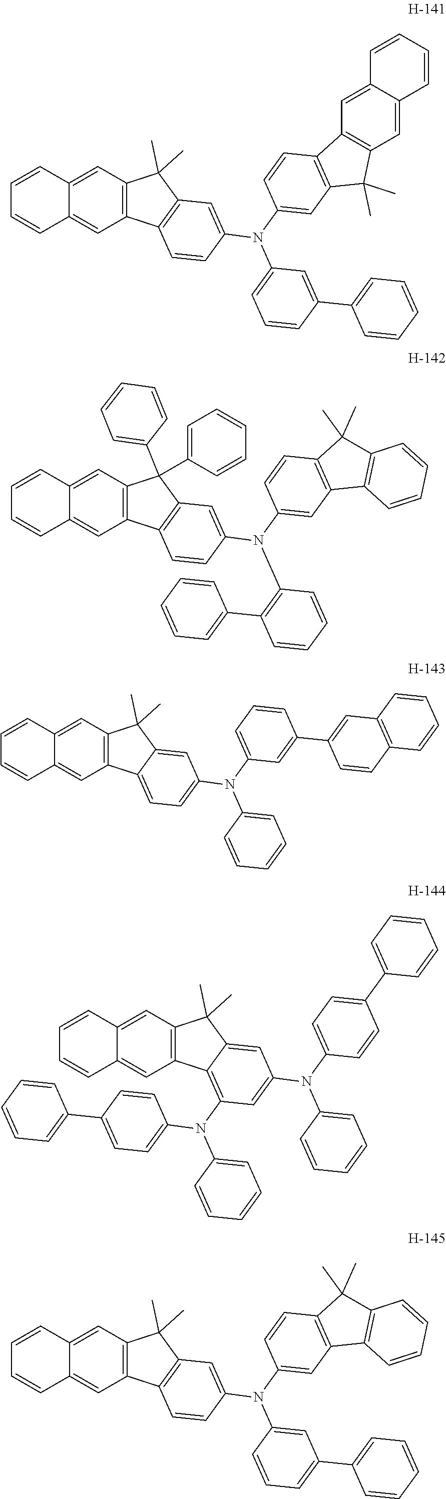 [SMILES: CC1(C)c2cc(N(c3cccc(-c4ccccc4)c3)c3ccc4c(c3)C(C)(C)c3cc5ccccc5cc3-4)ccc2-c2cc3ccccc3cc21 ; CC1(C)c2ccccc2-c2cc(N(c3ccc4c(c3)C(c3ccccc3)(c3ccccc3)c3cc5ccccc5cc3-4)c3ccccc3-c3ccccc3)ccc21 ; CC1(C)c2cc(N(c3ccccc3)c3cccc(-c4ccc5ccccc5c4)c3)ccc2-c2cc3ccccc3cc21 ; CC1(C)c2cc3ccccc3cc2-c2c(N(c3ccccc3)c3ccc(-c4ccccc4)cc3)cc(N(c3ccccc3)c3ccc(-c4ccccc4)cc3)cc21 ; CC1(C)c2ccccc2-c2cc(N(c3cccc(-c4ccccc4)c3)c3ccc4c(c3)C(C)(C)c3cc5ccccc5cc3-4)ccc21]

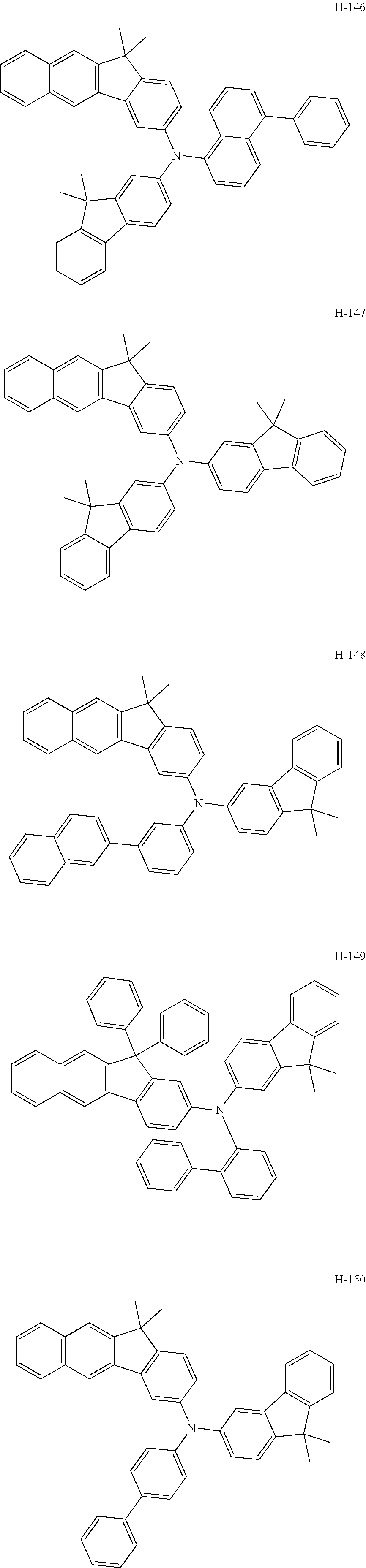 [SMILES: CC1(C)c2ccccc2-c2ccc(N(c3ccc4c(c3)-c3cc5ccccc5cc3C4(C)C)c3cccc4c(-c5ccccc5)cccc34)cc21 ; CC1(C)c2ccccc2-c2ccc(N(c3ccc4c(c3)-c3cc5ccccc5cc3C4(C)C)c3ccc4c(c3)C(C)(C)c3ccccc3-4)cc21 ; CC1(C)c2ccccc2-c2cc(N(c3cccc(-c4ccc5ccccc5c4)c3)c3ccc4c(c3)-c3cc5ccccc5cc3C4(C)C)ccc21 ; CC1(C)c2ccccc2-c2ccc(N(c3ccc4c(c3)C(c3ccccc3)(c3ccccc3)c3cc5ccccc5cc3-4)c3ccccc3-c3ccccc3)cc21 ; CC1(C)c2ccccc2-c2cc(N(c3ccc(-c4ccccc4)cc3)c3ccc4c(c3)-c3cc5ccccc5cc3C4(C)C)ccc21]

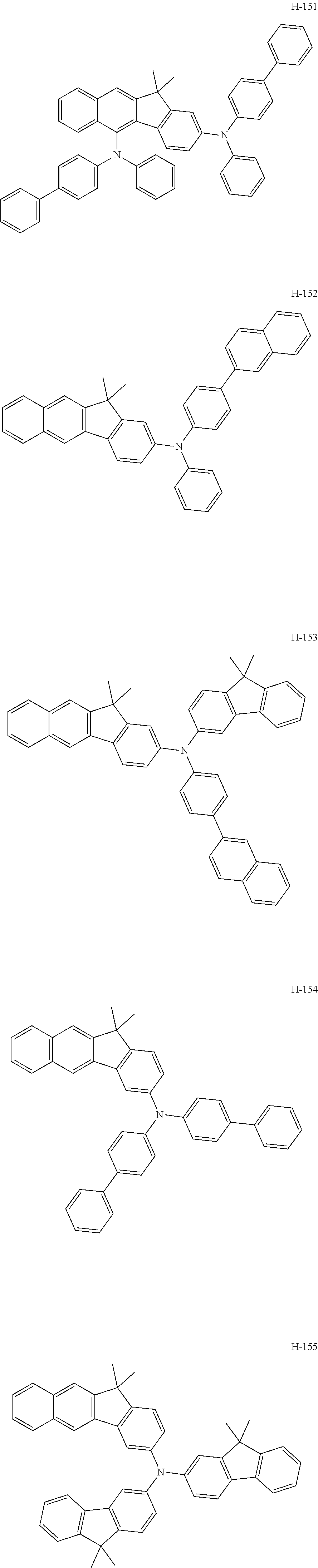 [SMILES: CC1(C)c2cc(N(c3ccccc3)c3ccc(-c4ccccc4)cc3)ccc2-c2c1cc1ccccc1c2N(c1ccccc1)c1ccc(-c2ccccc2)cc1 ; CC1(C)c2cc(N(c3ccccc3)c3ccc(-c4ccc5ccccc5c4)cc3)ccc2-c2cc3ccccc3cc21 ; CC1(C)c2ccccc2-c2cc(N(c3ccc(-c4ccc5ccccc5c4)cc3)c3ccc4c(c3)C(C)(C)c3cc5ccccc5cc3-4)ccc21 ; CC1(C)c2ccc(N(c3ccc(-c4ccccc4)cc3)c3ccc(-c4ccccc4)cc3)cc2-c2cc3ccccc3cc21 ; CC1(C)c2ccccc2-c2cc(N(c3ccc4c(c3)-c3cc5ccccc5cc3C4(C)C)c3ccc4c(c3)C(C)(C)c3ccccc3-4)ccc21]

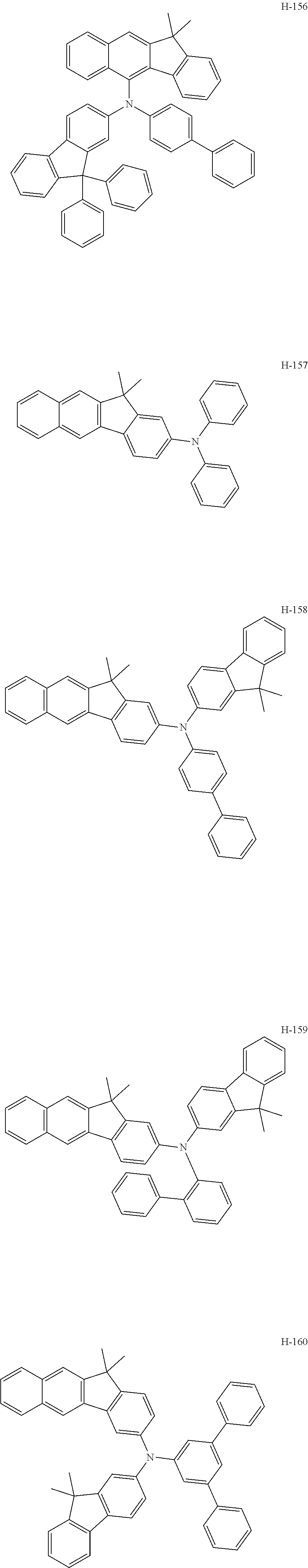 [SMILES: CC1(C)c2ccccc2-c2c1cc1ccccc1c2N(c1ccc(-c2ccccc2)cc1)c1ccc2c(c1)C(c1ccccc1)(c1ccccc1)c1ccccc1-2 ; CC1(C)c2cc(N(c3ccccc3)c3ccccc3)ccc2-c2cc3ccccc3cc21 ; CC1(C)c2ccccc2-c2ccc(N(c3ccc(-c4ccccc4)cc3)c3ccc4c(c3)C(C)(C)c3cc5ccccc5cc3-4)cc21 ; CC1(C)c2ccccc2-c2ccc(N(c3ccc4c(c3)C(C)(C)c3cc5ccccc5cc3-4)c3ccccc3-c3ccccc3)cc21 ; CC1(C)c2ccccc2-c2ccc(N(c3cc(-c4ccccc4)cc(-c4ccccc4)c3)c3ccc4c(c3)-c3cc5ccccc5cc3C4(C)C)cc21]

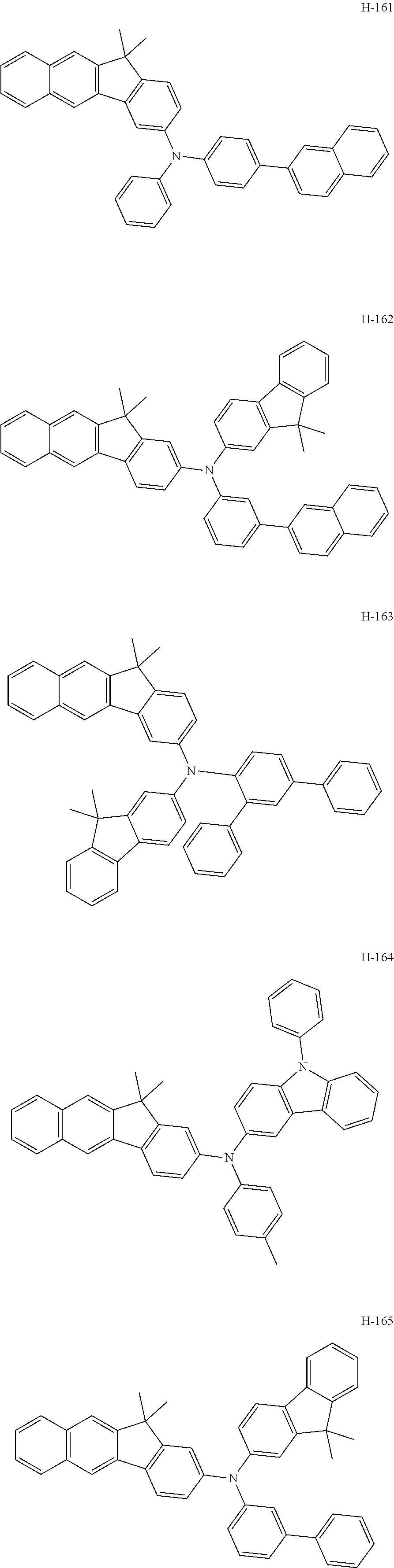 [SMILES: CC1(C)c2ccc(N(c3ccccc3)c3ccc(-c4ccc5ccccc5c4)cc3)cc2-c2cc3ccccc3cc21 ; CC1(C)c2ccccc2-c2ccc(N(c3cccc(-c4ccc5ccccc5c4)c3)c3ccc4c(c3)C(C)(C)c3cc5ccccc5cc3-4)cc21 ; CC1(C)c2ccccc2-c2ccc(N(c3ccc4c(c3)-c3cc5ccccc5cc3C4(C)C)c3ccc(-c4ccccc4)cc3-c3ccccc3)cc21 ; Cc1ccc(N(c2ccc3c(c2)C(C)(C)c2cc4ccccc4cc2-3)c2ccc3c(c2)c2ccccc2n3-c2ccccc2)cc1 ; CC1(C)c2ccccc2-c2ccc(N(c3cccc(-c4ccccc4)c3)c3ccc4c(c3)C(C)(C)c3cc5ccccc5cc3-4)cc21]

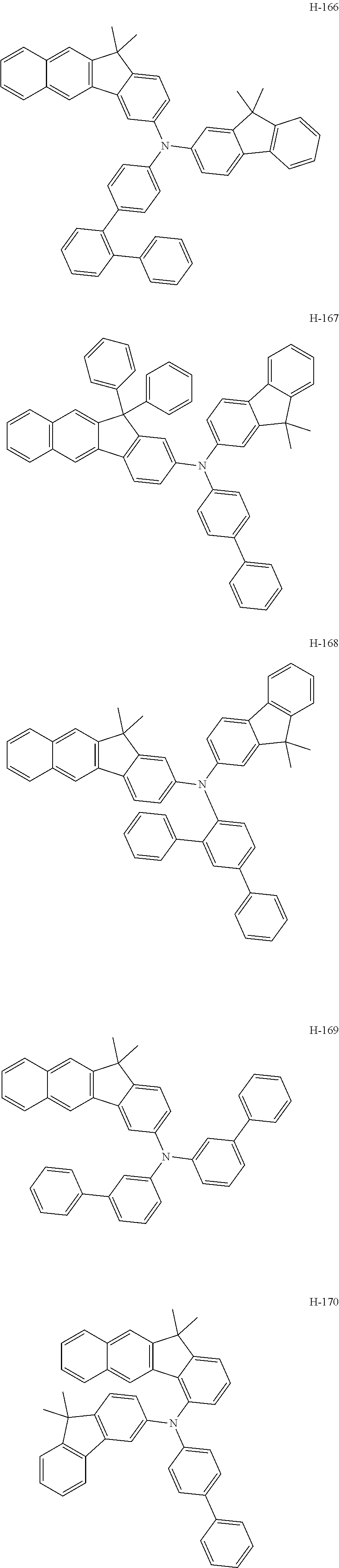 [SMILES: CC1(C)c2ccccc2-c2ccc(N(c3ccc(-c4ccccc4-c4ccccc4)cc3)c3ccc4c(c3)-c3cc5ccccc5cc3C4(C)C)cc21 ; CC1(C)c2ccccc2-c2ccc(N(c3ccc(-c4ccccc4)cc3)c3ccc4c(c3)C(c3ccccc3)(c3ccccc3)c3cc5ccccc5cc3-4)cc21 ; CC1(C)c2ccccc2-c2ccc(N(c3ccc4c(c3)C(C)(C)c3cc5ccccc5cc3-4)c3ccc(-c4ccccc4)cc3-c3ccccc3)cc21 ; CC1(C)c2ccc(N(c3cccc(-c4ccccc4)c3)c3cccc(-c4ccccc4)c3)cc2-c2cc3ccccc3cc21 ; CC1(C)c2ccccc2-c2cc(N(c3ccc(-c4ccccc4)cc3)c3cccc4c3-c3cc5ccccc5cc3C4(C)C)ccc21]

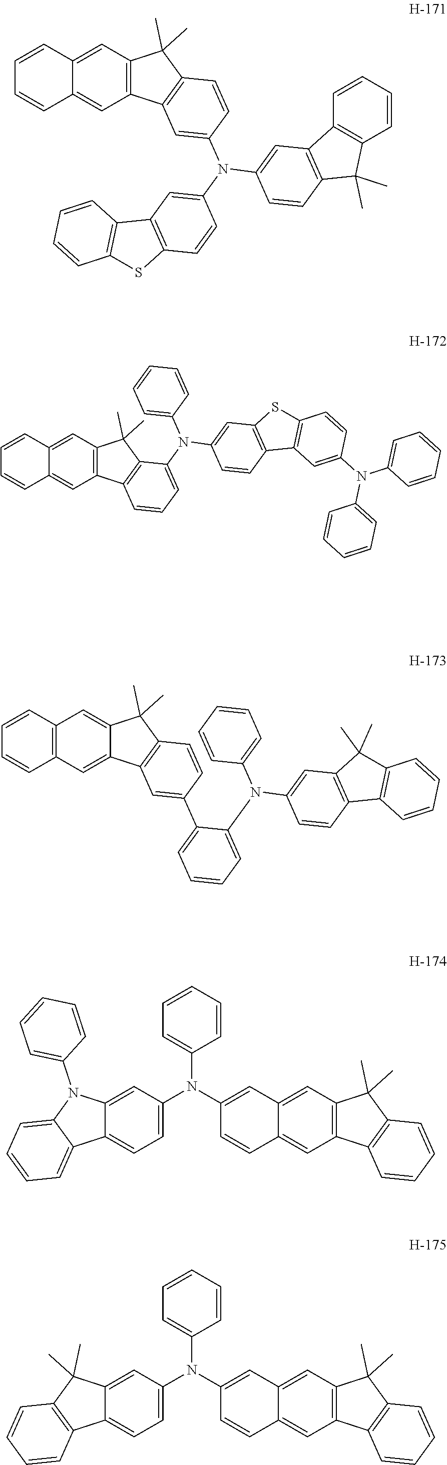 [SMILES: CC1(C)c2ccccc2-c2cc(N(c3ccc4c(c3)-c3cc5ccccc5cc3C4(C)C)c3ccc4sc5ccccc5c4c3)ccc21 ; CC1(C)c2cc3ccccc3cc2-c2cccc(N(c3ccccc3)c3ccc4c(c3)sc3ccc(N(c5ccccc5)c5ccccc5)cc34)c21 ; CC1(C)c2ccccc2-c2ccc(N(c3ccccc3)c3ccccc3-c3ccc4c(c3)-c3cc5ccccc5cc3C4(C)C)cc21 ; CC1(C)c2ccccc2-c2cc3ccc(N(c4ccccc4)c4ccc5c6ccccc6n(-c6ccccc6)c5c4)cc3cc21 ; CC1(C)c2ccccc2-c2ccc(N(c3ccccc3)c3ccc4cc5c(cc4c3)C(C)(C)c3ccccc3-5)cc21]

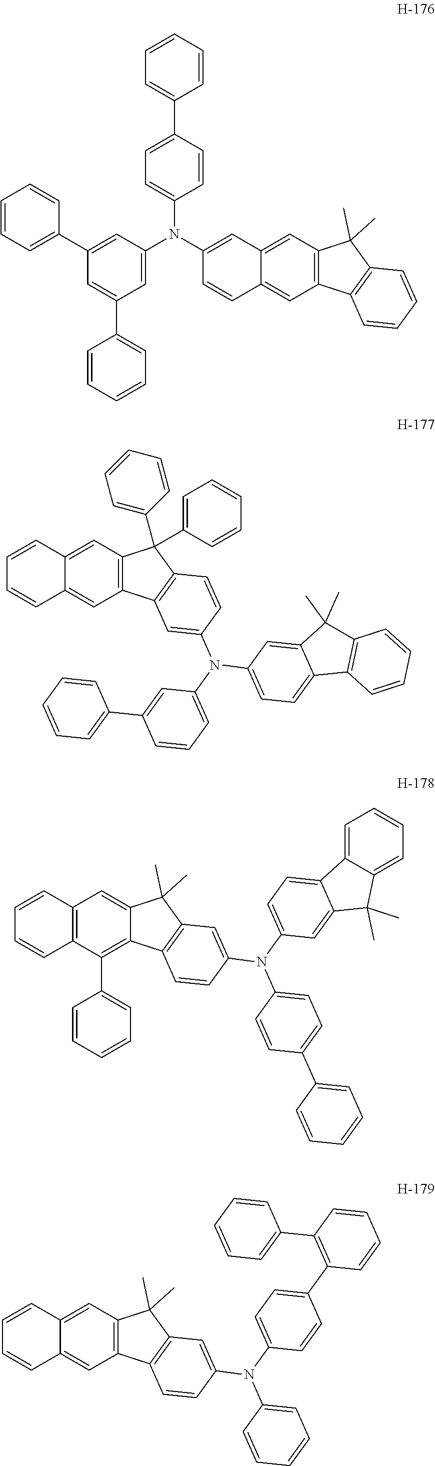 [SMILES: CC1(C)c2ccccc2-c2cc3ccc(N(c4ccc(-c5ccccc5)cc4)c4cc(-c5ccccc5)cc(-c5ccccc5)c4)cc3cc21 ; CC1(C)c2ccccc2-c2ccc(N(c3cccc(-c4ccccc4)c3)c3ccc4c(c3)-c3cc5ccccc5cc3C4(c3ccccc3)c3ccccc3)cc21 ; CC1(C)c2ccccc2-c2ccc(N(c3ccc(-c4ccccc4)cc3)c3ccc4c(c3)C(C)(C)c3cc5ccccc5c(-c5ccccc5)c3-4)cc21 ; CC1(C)c2cc(N(c3ccccc3)c3ccc(-c4ccccc4-c4ccccc4)cc3)ccc2-c2cc3ccccc3cc21]

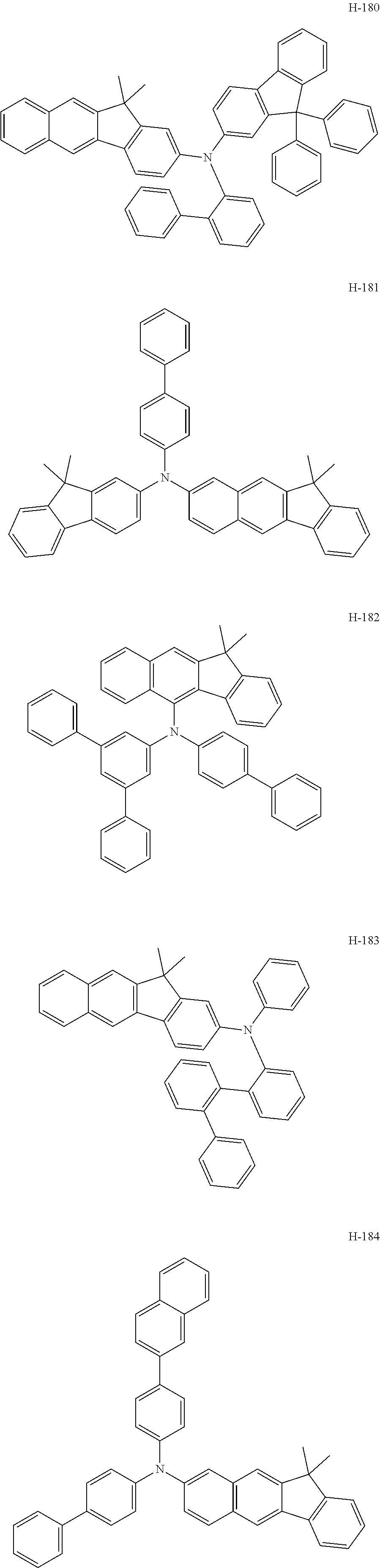 [SMILES: CC1(C)c2cc(N(c3ccc4c(c3)C(c3ccccc3)(c3ccccc3)c3ccccc3-4)c3ccccc3-c3ccccc3)ccc2-c2cc3ccccc3cc21 ; CC1(C)c2ccccc2-c2ccc(N(c3ccc(-c4ccccc4)cc3)c3ccc4cc5c(cc4c3)C(C)(C)c3ccccc3-5)cc21 ; CC1(C)c2ccccc2-c2c1cc1ccccc1c2N(c1ccc(-c2ccccc2)cc1)c1cc(-c2ccccc2)cc(-c2ccccc2)c1 ; CC1(C)c2cc(N(c3ccccc3)c3ccccc3-c3ccccc3-c3ccccc3)ccc2-c2cc3ccccc3cc21 ; CC1(C)c2ccccc2-c2cc3ccc(N(c4ccc(-c5ccccc5)cc4)c4ccc(-c5ccc6ccccc6c5)cc4)cc3cc21]

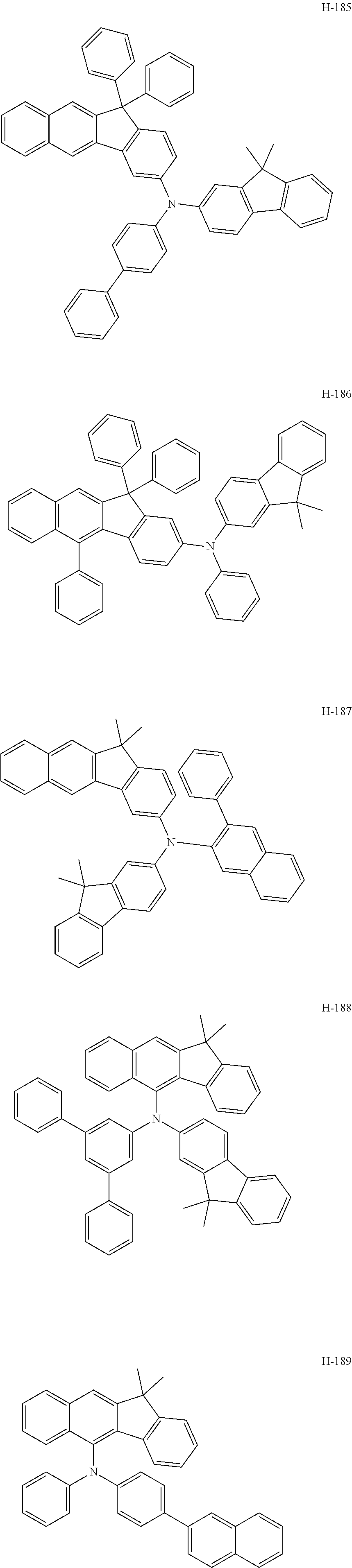 [SMILES: CC1(C)c2ccccc2-c2ccc(N(c3ccc(-c4ccccc4)cc3)c3ccc4c(c3)-c3cc5ccccc5cc3C4(c3ccccc3)c3ccccc3)cc21 ; CC1(C)c2ccccc2-c2ccc(N(c3ccccc3)c3ccc4c(c3)C(c3ccccc3)(c3ccccc3)c3cc5ccccc5c(-c5ccccc5)c3-4)cc21 ; CC1(C)c2ccccc2-c2ccc(N(c3ccc4c(c3)-c3cc5ccccc5cc3C4(C)C)c3cc4ccccc4cc3-c3ccccc3)cc21 ; CC1(C)c2ccccc2-c2ccc(N(c3cc(-c4ccccc4)cc(-c4ccccc4)c3)c3c4c(cc5ccccc35)C(C)(C)c3ccccc3-4)cc21 ; CC1(C)c2ccccc2-c2c1cc1ccccc1c2N(c1ccccc1)c1ccc(-c2ccc3ccccc3c2)cc1]

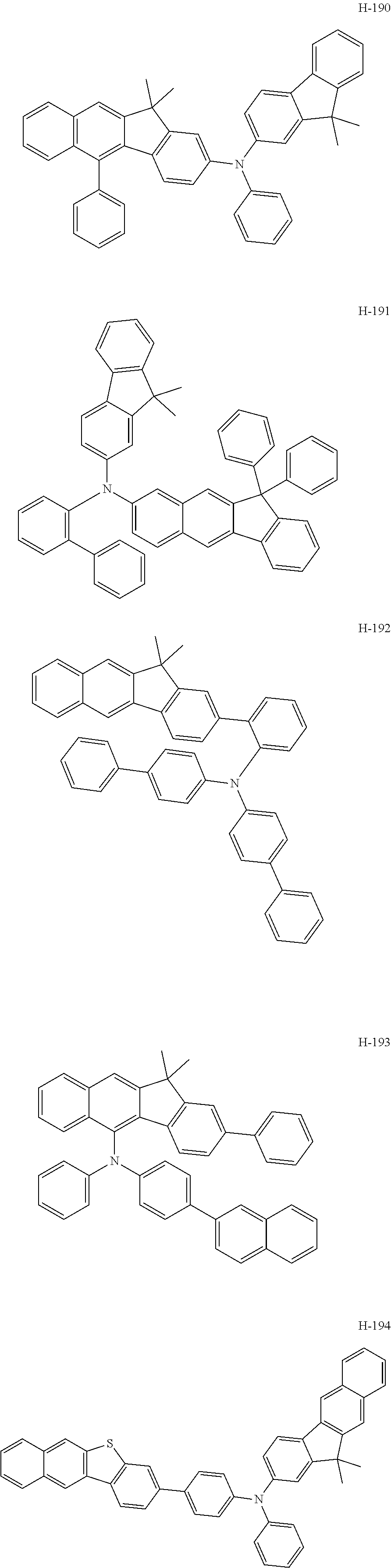 [SMILES: CC1(C)c2ccccc2-c2ccc(N(c3ccccc3)c3ccc4c(c3)C(C)(C)c3cc5ccccc5c(-c5ccccc5)c3-4)cc21 ; CC1(C)c2ccccc2-c2ccc(N(c3ccc4cc5c(cc4c3)C(c3ccccc3)(c3ccccc3)c3ccccc3-5)c3ccccc3-c3ccccc3)cc21 ; CC1(C)c2cc(-c3ccccc3N(c3ccc(-c4ccccc4)cc3)c3ccc(-c4ccccc4)cc3)ccc2-c2cc3ccccc3cc21 ; CC1(C)c2cc(-c3ccccc3)ccc2-c2c1cc1ccccc1c2N(c1ccccc1)c1ccc(-c2ccc3ccccc3c2)cc1 ; CC1(C)c2cc(N(c3ccccc3)c3ccc(-c4ccc5c(c4)sc4cc6ccccc6cc45)cc3)ccc2-c2cc3ccccc3cc21]

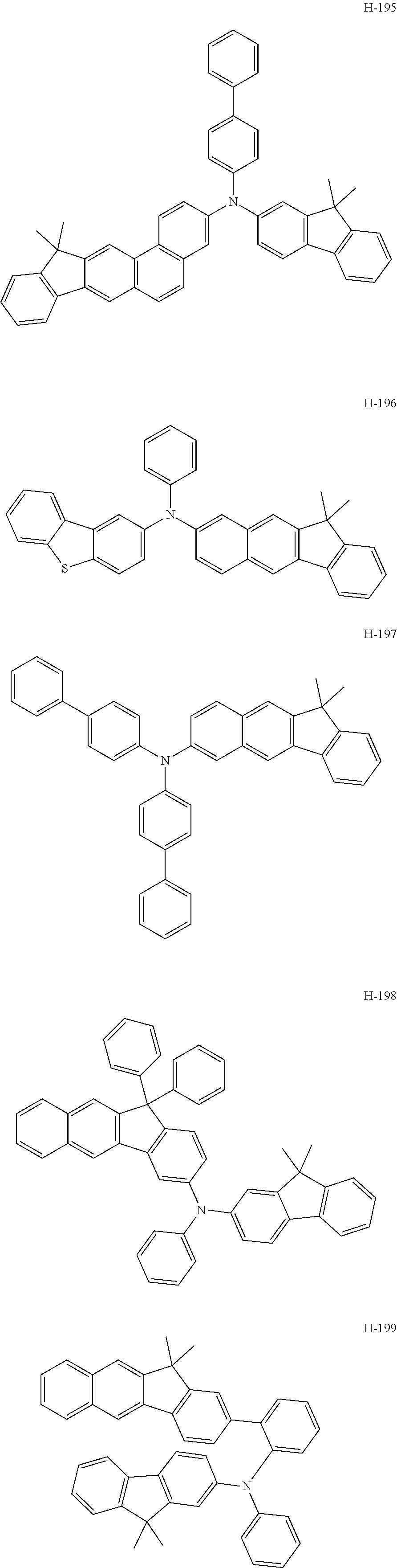 [SMILES: CC1(C)c2ccccc2-c2ccc(N(c3ccc(-c4ccccc4)cc3)c3ccc4c(ccc5cc6c(cc54)C(C)(C)c4ccccc4-6)c3)cc21 ; CC1(C)c2ccccc2-c2cc3ccc(N(c4ccccc4)c4ccc5sc6ccccc6c5c4)cc3cc21 ; CC1(C)c2ccccc2-c2cc3cc(N(c4ccc(-c5ccccc5)cc4)c4ccc(-c5ccccc5)cc4)ccc3cc21 ; CC1(C)c2ccccc2-c2ccc(N(c3ccccc3)c3ccc4c(c3)-c3cc5ccccc5cc3C4(c3ccccc3)c3ccccc3)cc21 ; CC1(C)c2ccccc2-c2ccc(N(c3ccccc3)c3ccccc3-c3ccc4c(c3)C(C)(C)c3cc5ccccc5cc3-4)cc21]

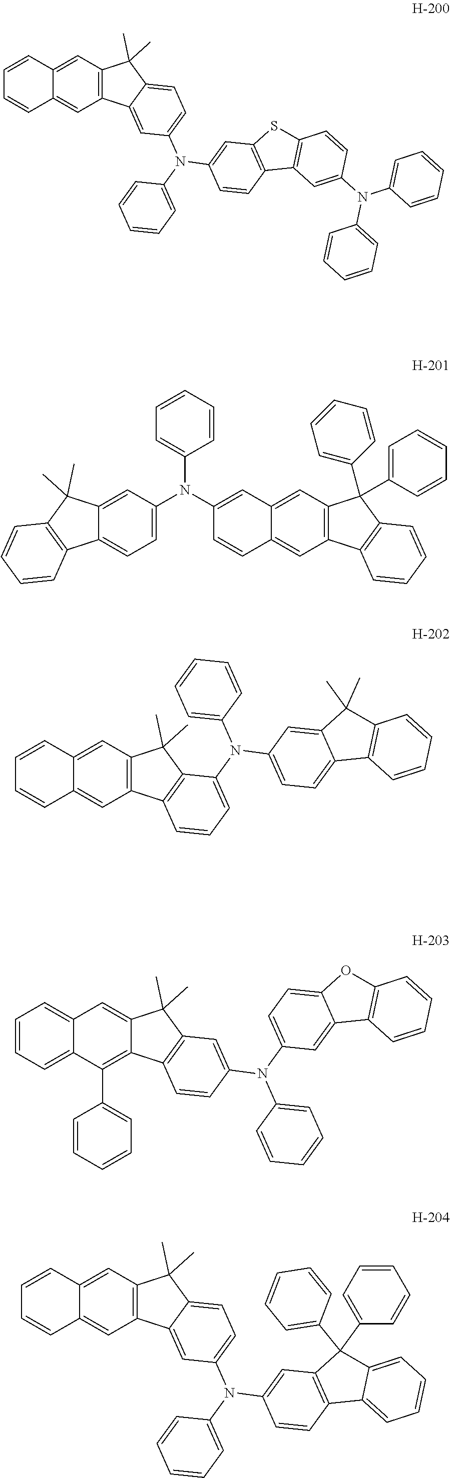 [SMILES: CC1(C)c2ccc(N(c3ccccc3)c3ccc4c(c3)sc3ccc(N(c5ccccc5)c5ccccc5)cc34)cc2-c2cc3ccccc3cc21 ; CC1(C)c2ccccc2-c2ccc(N(c3ccccc3)c3ccc4cc5c(cc4c3)C(c3ccccc3)(c3ccccc3)c3ccccc3-5)cc21 ; CC1(C)c2ccccc2-c2ccc(N(c3ccccc3)c3cccc4c3C(C)(C)c3cc5ccccc5cc3-4)cc21 ; CC1(C)c2cc(N(c3ccccc3)c3ccc4oc5ccccc5c4c3)ccc2-c2c1cc1ccccc1c2-c1ccccc1 ; CC1(C)c2ccc(N(c3ccccc3)c3ccc4c(c3)C(c3ccccc3)(c3ccccc3)c3ccccc3-4)cc2-c2cc3ccccc3cc21]

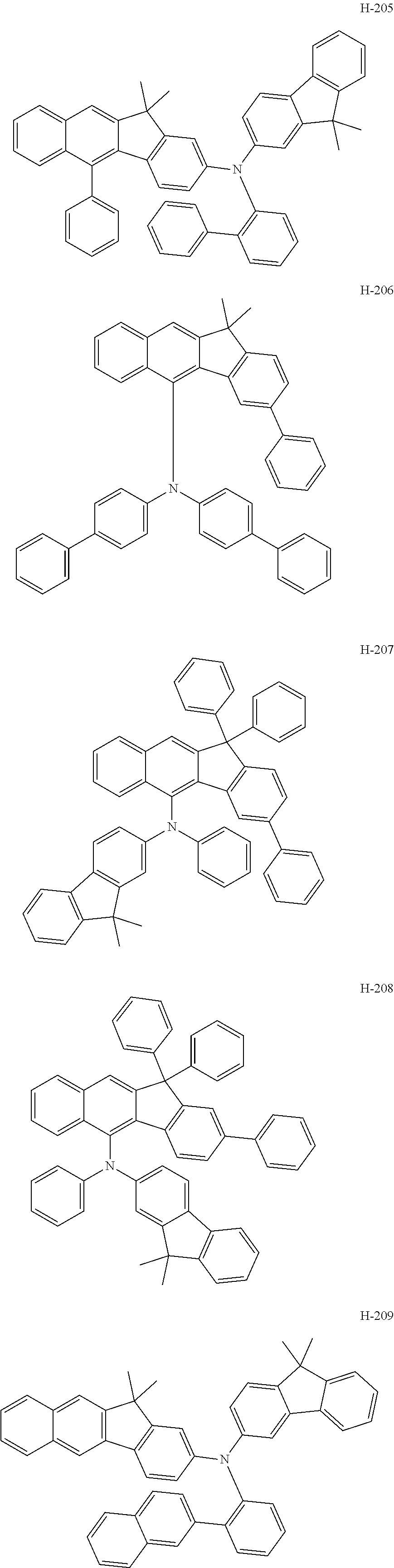 [SMILES: CC1(C)c2ccccc2-c2ccc(N(c3ccc4c(c3)C(C)(C)c3cc5ccccc5c(-c5ccccc5)c3-4)c3ccccc3-c3ccccc3)cc21 ; CC1(C)c2ccc(-c3ccccc3)cc2-c2c1cc1ccccc1c2N(c1ccc(-c2ccccc2)cc1)c1ccc(-c2ccccc2)cc1 ; CC1(C)c2ccccc2-c2ccc(N(c3ccccc3)c3c4c(cc5ccccc35)C(c3ccccc3)(c3ccccc3)c3ccc(-c5ccccc5)cc3-4)cc21 ; CC1(C)c2ccccc2-c2ccc(N(c3ccccc3)c3c4c(cc5ccccc35)C(c3ccccc3)(c3ccccc3)c3cc(-c5ccccc5)ccc3-4)cc21 ; CC1(C)c2ccccc2-c2cc(N(c3ccc4c(c3)C(C)(C)c3cc5ccccc5cc3-4)c3ccccc3-c3ccc4ccccc4c3)ccc21]

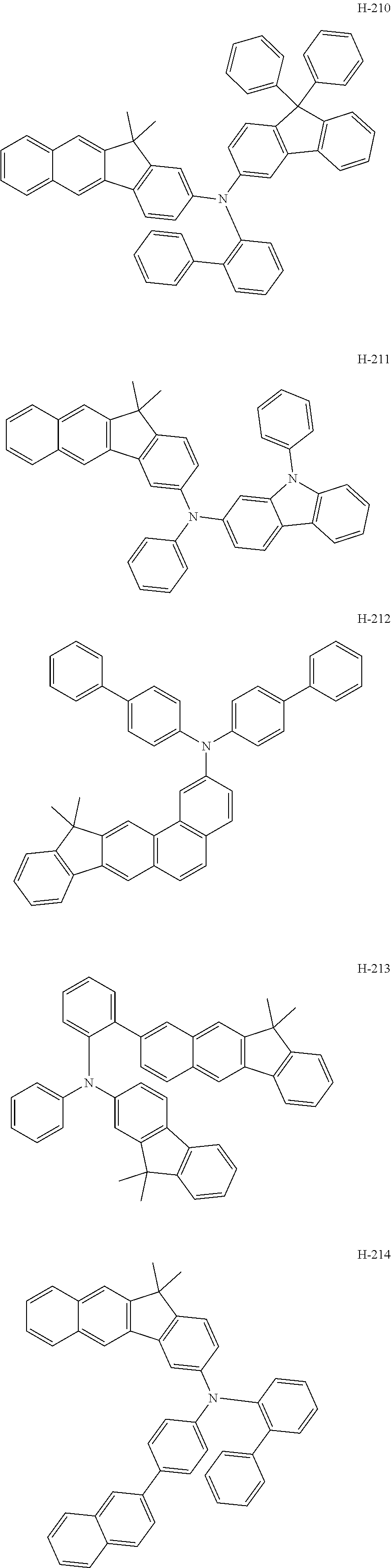 [SMILES: CC1(C)c2cc(N(c3ccc4c(c3)-c3ccccc3C4(c3ccccc3)c3ccccc3)c3ccccc3-c3ccccc3)ccc2-c2cc3ccccc3cc21 ; CC1(C)c2ccc(N(c3ccccc3)c3ccc4c5ccccc5n(-c5ccccc5)c4c3)cc2-c2cc3ccccc3cc21 ; CC1(C)c2ccccc2-c2cc3ccc4ccc(N(c5ccc(-c6ccccc6)cc5)c5ccc(-c6ccccc6)cc5)cc4c3cc21 ; CC1(C)c2ccccc2-c2ccc(N(c3ccccc3)c3ccccc3-c3ccc4cc5c(cc4c3)C(C)(C)c3ccccc3-5)cc21 ; CC1(C)c2ccc(N(c3ccc(-c4ccc5ccccc5c4)cc3)c3ccccc3-c3ccccc3)cc2-c2cc3ccccc3cc21]

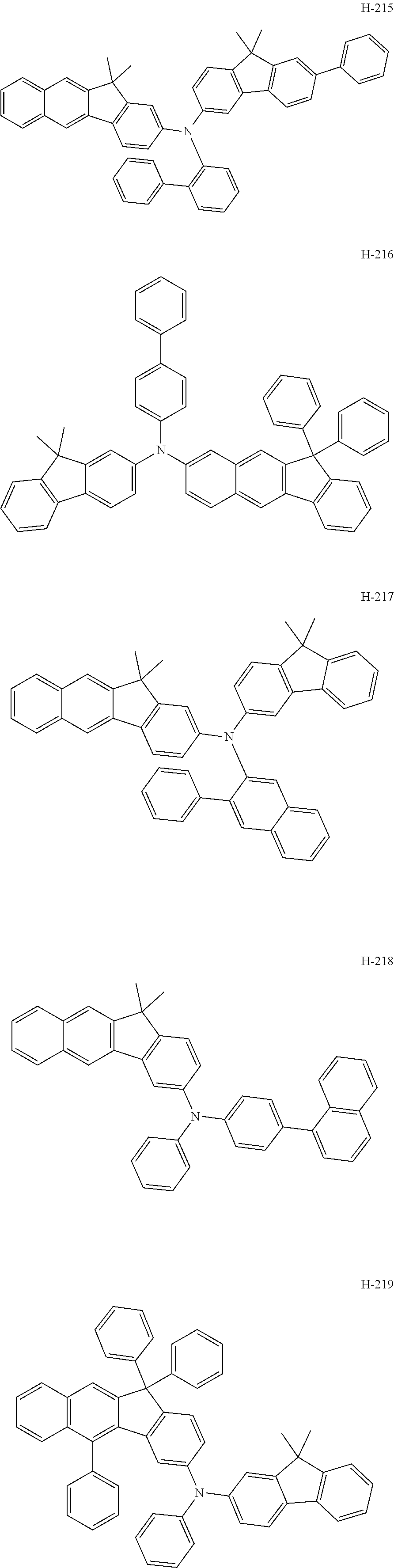 [SMILES: CC1(C)c2ccc(N(c3ccc4c(c3)C(C)(C)c3cc5ccccc5cc3-4)c3ccccc3-c3ccccc3)cc2-c2ccc(-c3ccccc3)cc21 ; CC1(C)c2ccccc2-c2ccc(N(c3ccc(-c4ccccc4)cc3)c3ccc4cc5c(cc4c3)C(c3ccccc3)(c3ccccc3)c3ccccc3-5)cc21 ; CC1(C)c2ccccc2-c2cc(N(c3ccc4c(c3)C(C)(C)c3cc5ccccc5cc3-4)c3cc4ccccc4cc3-c3ccccc3)ccc21 ; CC1(C)c2ccc(N(c3ccccc3)c3ccc(-c4cccc5ccccc45)cc3)cc2-c2cc3ccccc3cc21 ; CC1(C)c2ccccc2-c2ccc(N(c3ccccc3)c3ccc4c(c3)-c3c(cc5ccccc5c3-c3ccccc3)C4(c3ccccc3)c3ccccc3)cc21]

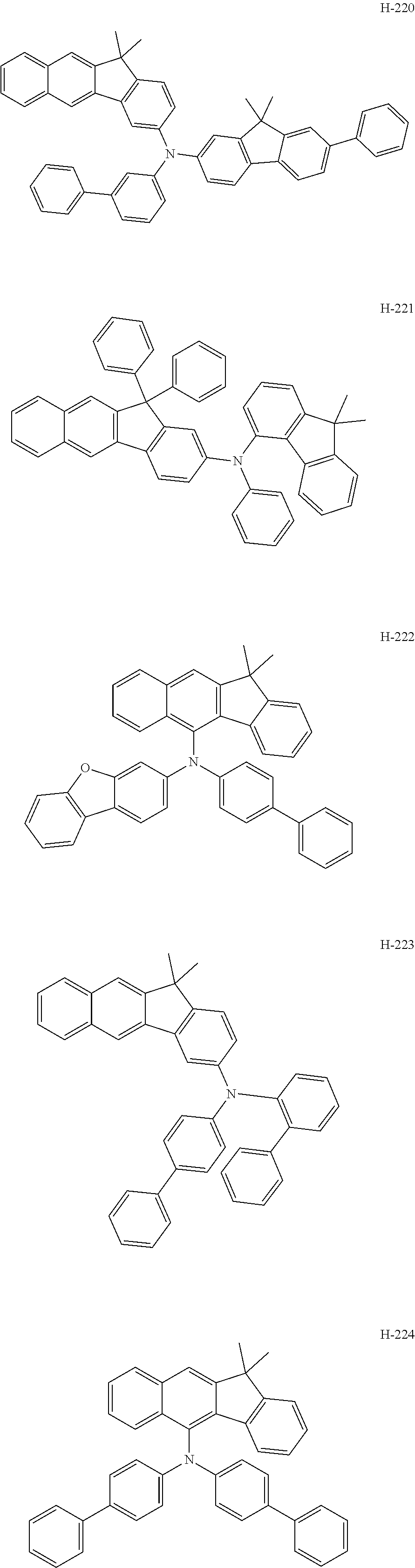 [SMILES: CC1(C)c2cc(-c3ccccc3)ccc2-c2ccc(N(c3cccc(-c4ccccc4)c3)c3ccc4c(c3)-c3cc5ccccc5cc3C4(C)C)cc21 ; CC1(C)c2ccccc2-c2c(N(c3ccccc3)c3ccc4c(c3)C(c3ccccc3)(c3ccccc3)c3cc5ccccc5cc3-4)cccc21 ; CC1(C)c2ccccc2-c2c1cc1ccccc1c2N(c1ccc(-c2ccccc2)cc1)c1ccc2c(c1)oc1ccccc12 ; CC1(C)c2ccc(N(c3ccc(-c4ccccc4)cc3)c3ccccc3-c3ccccc3)cc2-c2cc3ccccc3cc21 ; CC1(C)c2ccccc2-c2c1cc1ccccc1c2N(c1ccc(-c2ccccc2)cc1)c1ccc(-c2ccccc2)cc1]

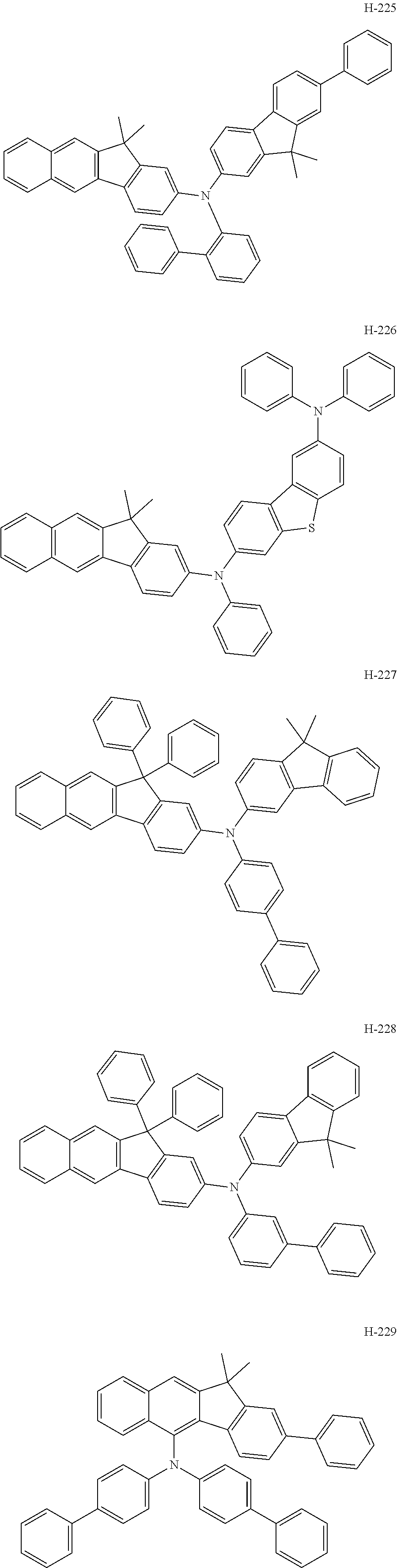 [SMILES: CC1(C)c2cc(-c3ccccc3)ccc2-c2ccc(N(c3ccc4c(c3)C(C)(C)c3cc5ccccc5cc3-4)c3ccccc3-c3ccccc3)cc21 ; CC1(C)c2cc(N(c3ccccc3)c3ccc4c(c3)sc3ccc(N(c5ccccc5)c5ccccc5)cc34)ccc2-c2cc3ccccc3cc21 ; CC1(C)c2ccccc2-c2cc(N(c3ccc(-c4ccccc4)cc3)c3ccc4c(c3)C(c3ccccc3)(c3ccccc3)c3cc5ccccc5cc3-4)ccc21 ; CC1(C)c2ccccc2-c2ccc(N(c3cccc(-c4ccccc4)c3)c3ccc4c(c3)C(c3ccccc3)(c3ccccc3)c3cc5ccccc5cc3-4)cc21 ; CC1(C)c2cc(-c3ccccc3)ccc2-c2c1cc1ccccc1c2N(c1ccc(-c2ccccc2)cc1)c1ccc(-c2ccccc2)cc1]

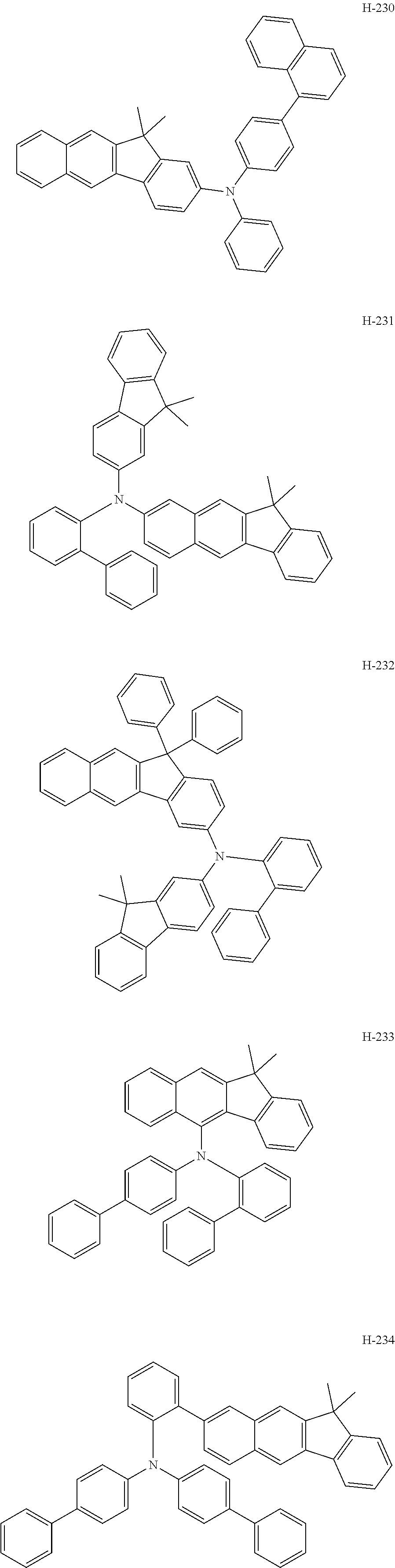 [SMILES: CC1(C)c2cc(N(c3ccccc3)c3ccc(-c4cccc5ccccc45)cc3)ccc2-c2cc3ccccc3cc21 ; CC1(C)c2ccccc2-c2ccc(N(c3ccc4cc5c(cc4c3)C(C)(C)c3ccccc3-5)c3ccccc3-c3ccccc3)cc21 ; CC1(C)c2ccccc2-c2ccc(N(c3ccc4c(c3)-c3cc5ccccc5cc3C4(c3ccccc3)c3ccccc3)c3ccccc3-c3ccccc3)cc21 ; CC1(C)c2ccccc2-c2c1cc1ccccc1c2N(c1ccc(-c2ccccc2)cc1)c1ccccc1-c1ccccc1 ; CC1(C)c2ccccc2-c2cc3ccc(-c4ccccc4N(c4ccc(-c5ccccc5)cc4)c4ccc(-c5ccccc5)cc4)cc3cc21]

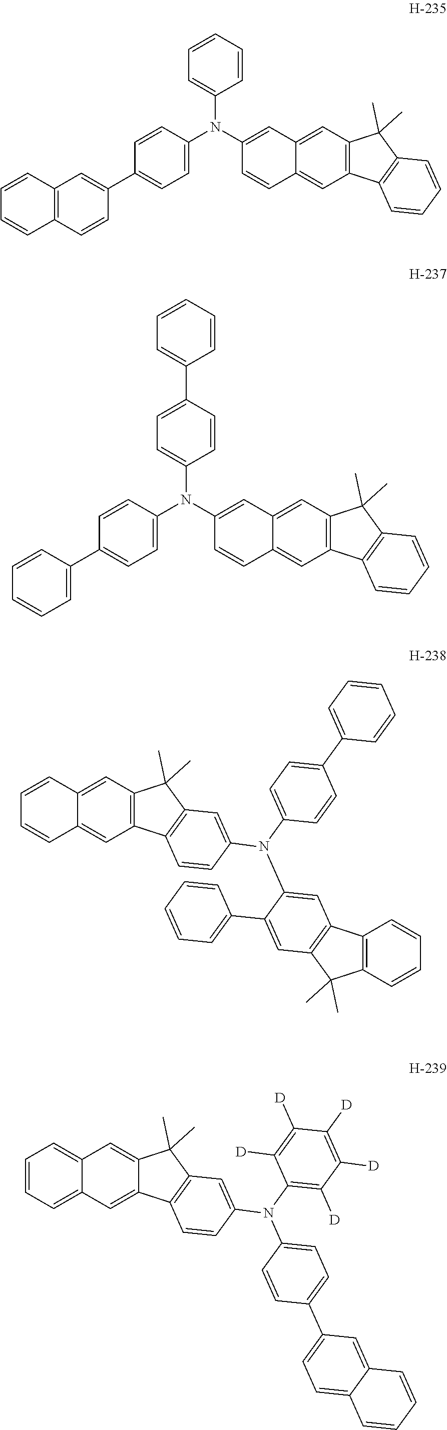 [SMILES: CC1(C)c2ccccc2-c2cc3ccc(N(c4ccccc4)c4ccc(-c5ccc6ccccc6c5)cc4)cc3cc21 ; CC1(C)c2ccccc2-c2cc3ccc(N(c4ccc(-c5ccccc5)cc4)c4ccc(-c5ccccc5)cc4)cc3cc21 ; CC1(C)c2ccccc2-c2cc(N(c3ccc(-c4ccccc4)cc3)c3ccc4c(c3)C(C)(C)c3cc5ccccc5cc3-4)c(-c3ccccc3)cc21 ; [2H]c1c([2H])c([2H])c(N(c2ccc(-c3ccc4ccccc4c3)cc2)c2ccc3c(c2)C(C)(C)c2cc4ccccc4cc2-3)c([2H])c1[2H]]

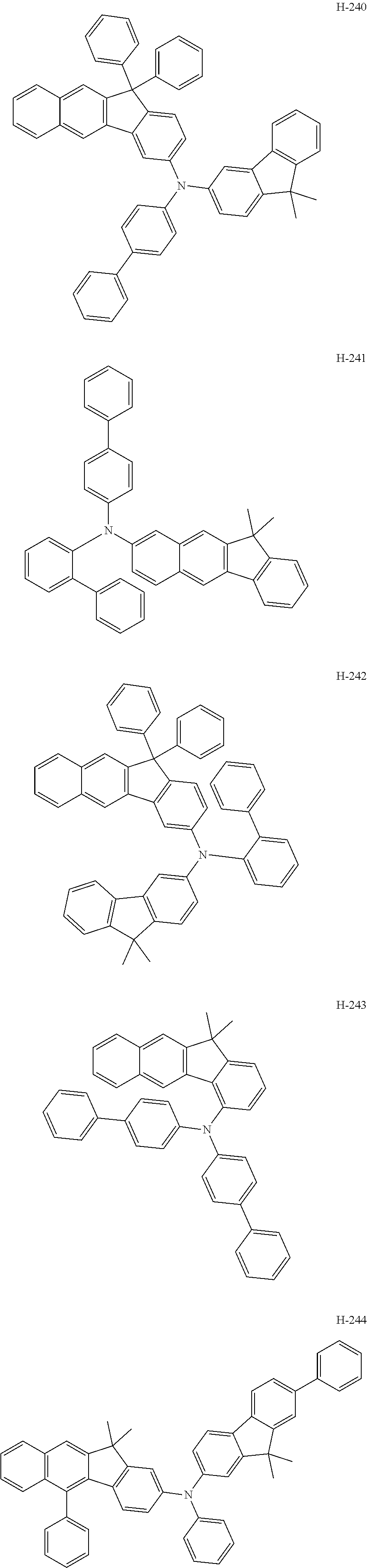 [SMILES: CC1(C)c2ccccc2-c2cc(N(c3ccc(-c4ccccc4)cc3)c3ccc4c(c3)-c3cc5ccccc5cc3C4(c3ccccc3)c3ccccc3)ccc21 ; CC1(C)c2ccccc2-c2cc3ccc(N(c4ccc(-c5ccccc5)cc4)c4ccccc4-c4ccccc4)cc3cc21 ; CC1(C)c2ccccc2-c2cc(N(c3ccc4c(c3)-c3cc5ccccc5cc3C4(c3ccccc3)c3ccccc3)c3ccccc3-c3ccccc3)ccc21 ; CC1(C)c2cc3ccccc3cc2-c2c(N(c3ccc(-c4ccccc4)cc3)c3ccc(-c4ccccc4)cc3)cccc21 ; CC1(C)c2cc(-c3ccccc3)ccc2-c2ccc(N(c3ccccc3)c3ccc4c(c3)C(C)(C)c3cc5ccccc5c(-c5ccccc5)c3-4)cc21]

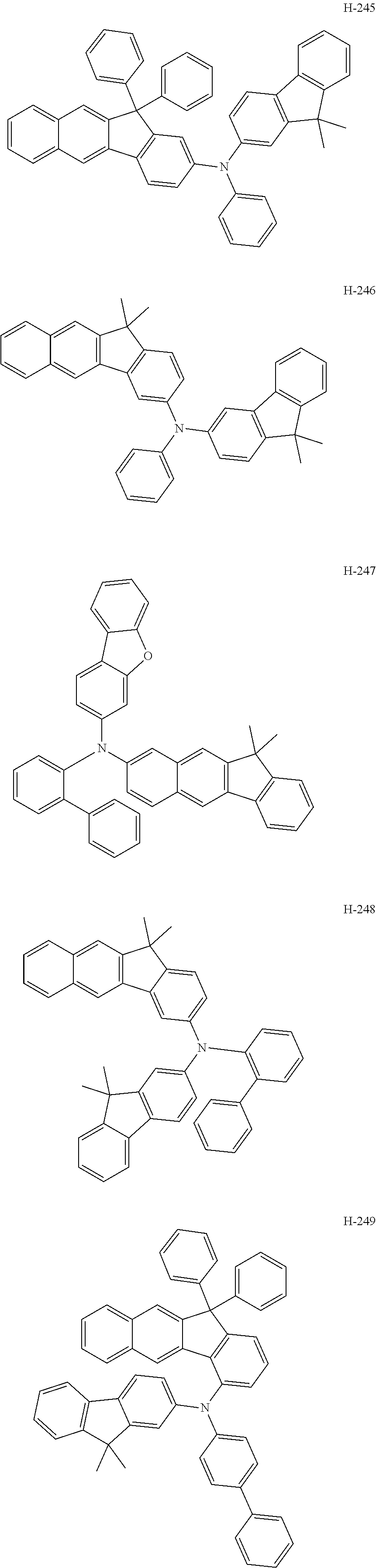 [SMILES: CC1(C)c2ccccc2-c2ccc(N(c3ccccc3)c3ccc4c(c3)C(c3ccccc3)(c3ccccc3)c3cc5ccccc5cc3-4)cc21 ; CC1(C)c2ccccc2-c2cc(N(c3ccccc3)c3ccc4c(c3)-c3cc5ccccc5cc3C4(C)C)ccc21 ; CC1(C)c2ccccc2-c2cc3ccc(N(c4ccc5c(c4)oc4ccccc45)c4ccccc4-c4ccccc4)cc3cc21 ; CC1(C)c2ccccc2-c2ccc(N(c3ccc4c(c3)-c3cc5ccccc5cc3C4(C)C)c3ccccc3-c3ccccc3)cc21 ; CC1(C)c2ccccc2-c2ccc(N(c3ccc(-c4ccccc4)cc3)c3cccc4c3-c3cc5ccccc5cc3C4(c3ccccc3)c3ccccc3)cc21]

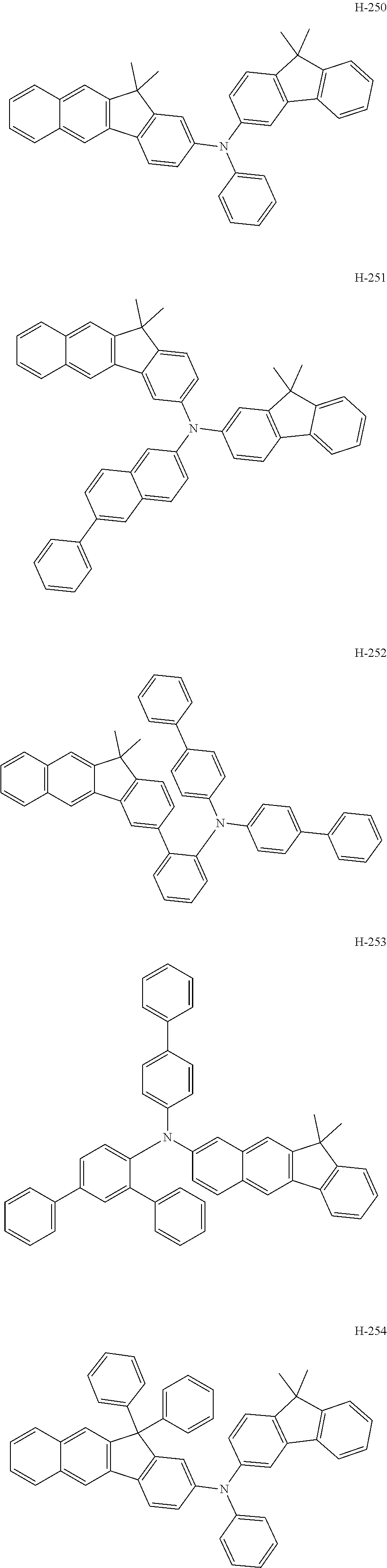 [SMILES: CC1(C)c2ccccc2-c2cc(N(c3ccccc3)c3ccc4c(c3)C(C)(C)c3cc5ccccc5cc3-4)ccc21 ; CC1(C)c2ccccc2-c2ccc(N(c3ccc4c(c3)-c3cc5ccccc5cc3C4(C)C)c3ccc4cc(-c5ccccc5)ccc4c3)cc21 ; CC1(C)c2ccc(-c3ccccc3N(c3ccc(-c4ccccc4)cc3)c3ccc(-c4ccccc4)cc3)cc2-c2cc3ccccc3cc21 ; CC1(C)c2ccccc2-c2cc3ccc(N(c4ccc(-c5ccccc5)cc4)c4ccc(-c5ccccc5)cc4-c4ccccc4)cc3cc21 ; CC1(C)c2ccccc2-c2cc(N(c3ccccc3)c3ccc4c(c3)C(c3ccccc3)(c3ccccc3)c3cc5ccccc5cc3-4)ccc21]

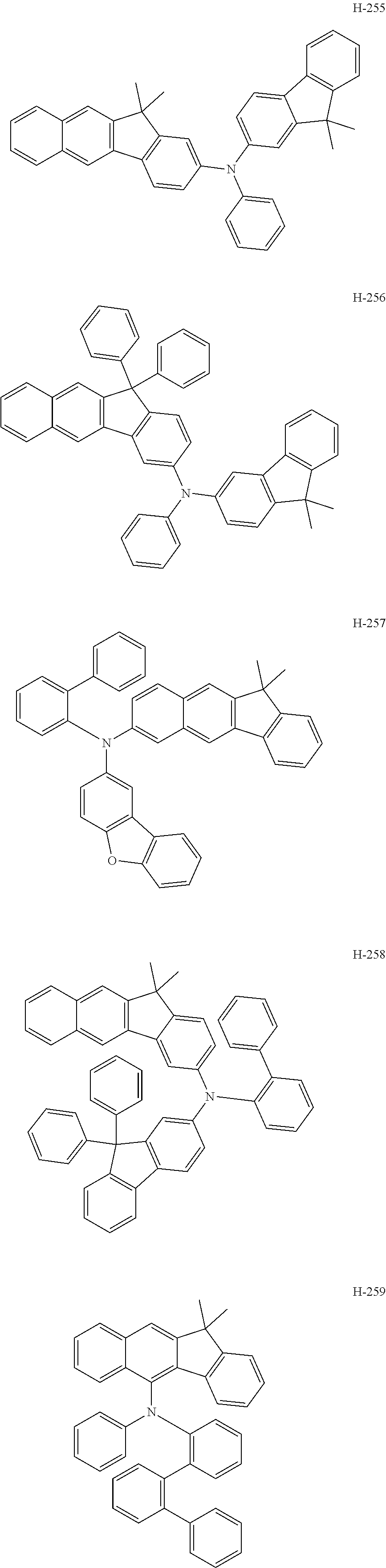 [SMILES: CC1(C)c2ccccc2-c2ccc(N(c3ccccc3)c3ccc4c(c3)C(C)(C)c3cc5ccccc5cc3-4)cc21 ; CC1(C)c2ccccc2-c2cc(N(c3ccccc3)c3ccc4c(c3)-c3cc5ccccc5cc3C4(c3ccccc3)c3ccccc3)ccc21 ; CC1(C)c2ccccc2-c2cc3cc(N(c4ccc5oc6ccccc6c5c4)c4ccccc4-c4ccccc4)ccc3cc21 ; CC1(C)c2ccc(N(c3ccc4c(c3)C(c3ccccc3)(c3ccccc3)c3ccccc3-4)c3ccccc3-c3ccccc3)cc2-c2cc3ccccc3cc21 ; CC1(C)c2ccccc2-c2c1cc1ccccc1c2N(c1ccccc1)c1ccccc1-c1ccccc1-c1ccccc1]

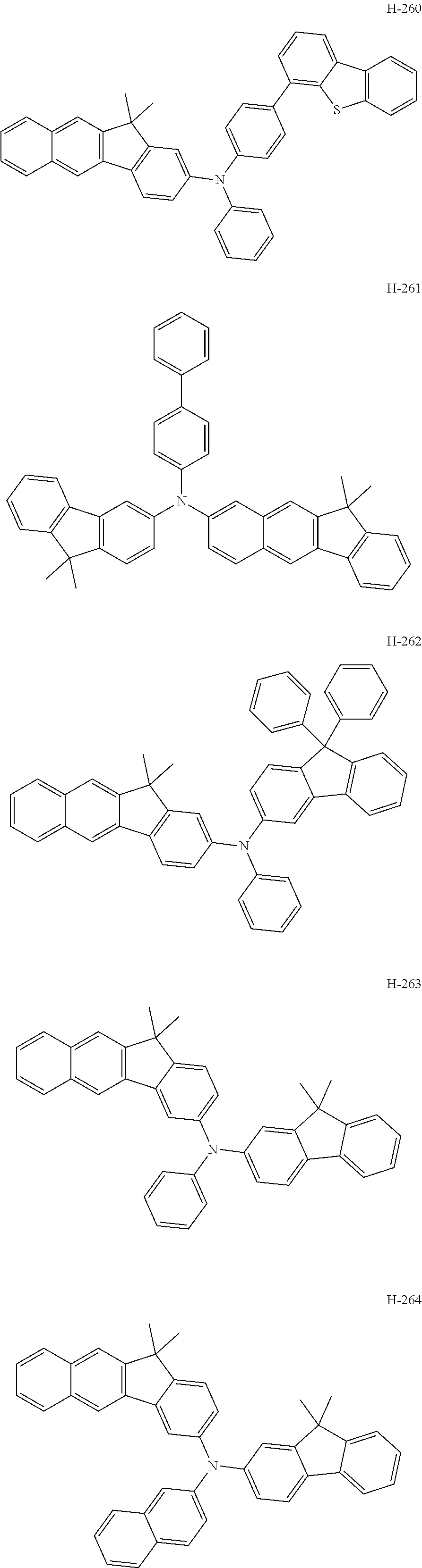 [SMILES: CC1(C)c2cc(N(c3ccccc3)c3ccc(-c4cccc5c4sc4ccccc45)cc3)ccc2-c2cc3ccccc3cc21 ; CC1(C)c2ccccc2-c2cc(N(c3ccc(-c4ccccc4)cc3)c3ccc4cc5c(cc4c3)C(C)(C)c3ccccc3-5)ccc21 ; CC1(C)c2cc(N(c3ccccc3)c3ccc4c(c3)-c3ccccc3C4(c3ccccc3)c3ccccc3)ccc2-c2cc3ccccc3cc21 ; CC1(C)c2ccccc2-c2ccc(N(c3ccccc3)c3ccc4c(c3)-c3cc5ccccc5cc3C4(C)C)cc21 ; CC1(C)c2ccccc2-c2ccc(N(c3ccc4c(c3)-c3cc5ccccc5cc3C4(C)C)c3ccc4ccccc4c3)cc21]

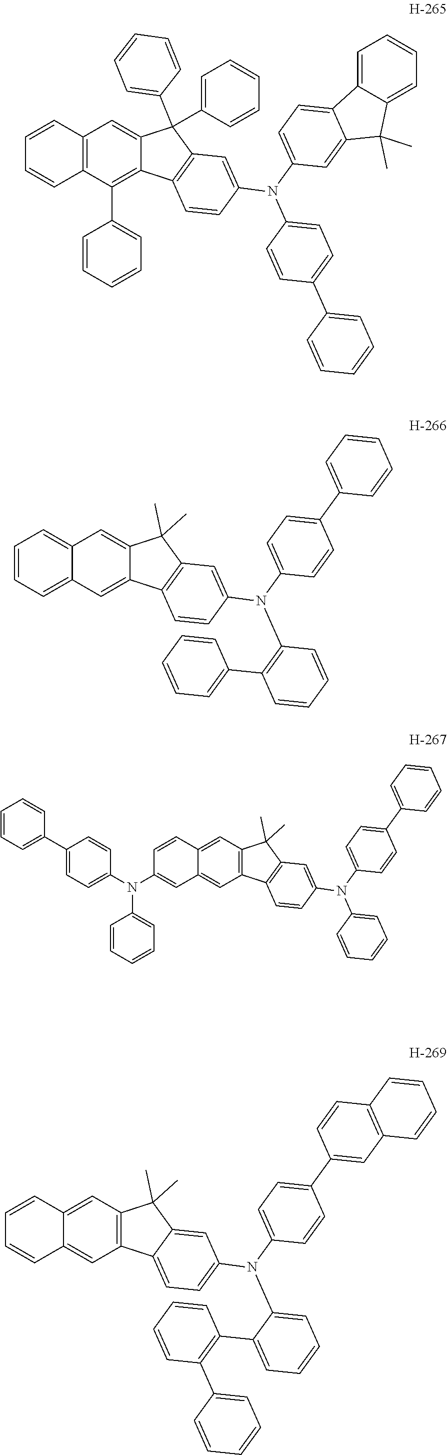 [SMILES: CC1(C)c2ccccc2-c2ccc(N(c3ccc(-c4ccccc4)cc3)c3ccc4c(c3)C(c3ccccc3)(c3ccccc3)c3cc5ccccc5c(-c5ccccc5)c3-4)cc21 ; CC1(C)c2cc(N(c3ccc(-c4ccccc4)cc3)c3ccccc3-c3ccccc3)ccc2-c2cc3ccccc3cc21 ; CC1(C)c2cc(N(c3ccccc3)c3ccc(-c4ccccc4)cc3)ccc2-c2cc3cc(N(c4ccccc4)c4ccc(-c5ccccc5)cc4)ccc3cc21 ; CC1(C)c2cc(N(c3ccc(-c4ccc5ccccc5c4)cc3)c3ccccc3-c3ccccc3-c3ccccc3)ccc2-c2cc3ccccc3cc21]

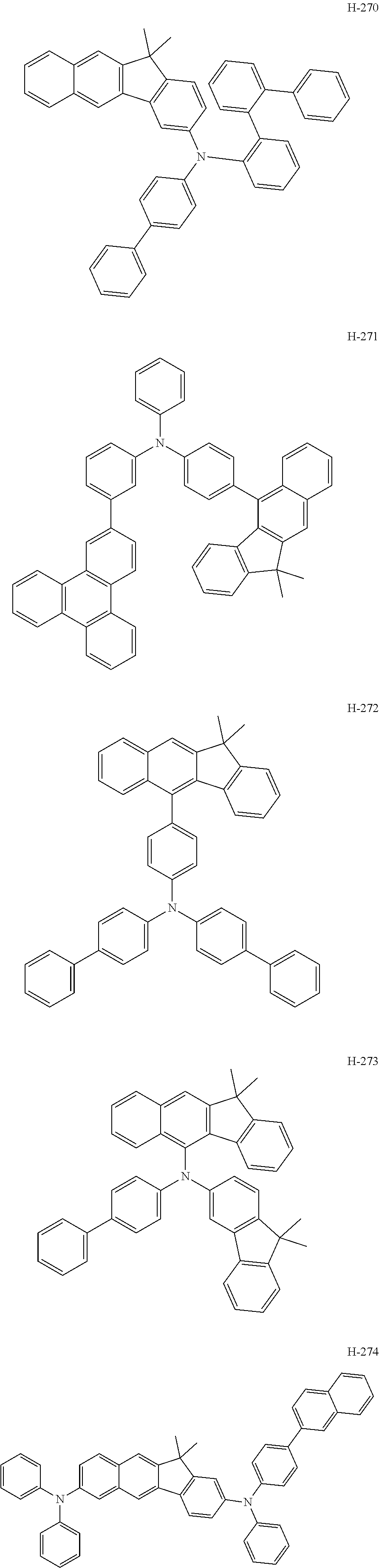 [SMILES: CC1(C)c2ccc(N(c3ccc(-c4ccccc4)cc3)c3ccccc3-c3ccccc3-c3ccccc3)cc2-c2cc3ccccc3cc21 ; CC1(C)c2ccccc2-c2c1cc1ccccc1c2-c1ccc(N(c2ccccc2)c2cccc(-c3ccc4c5ccccc5c5ccccc5c4c3)c2)cc1 ; CC1(C)c2ccccc2-c2c1cc1ccccc1c2-c1ccc(N(c2ccc(-c3ccccc3)cc2)c2ccc(-c3ccccc3)cc2)cc1 ; CC1(C)c2ccccc2-c2cc(N(c3ccc(-c4ccccc4)cc3)c3c4c(cc5ccccc35)C(C)(C)c3ccccc3-4)ccc21 ; CC1(C)c2cc(N(c3ccccc3)c3ccc(-c4ccc5ccccc5c4)cc3)ccc2-c2cc3cc(N(c4ccccc4)c4ccccc4)ccc3cc21]

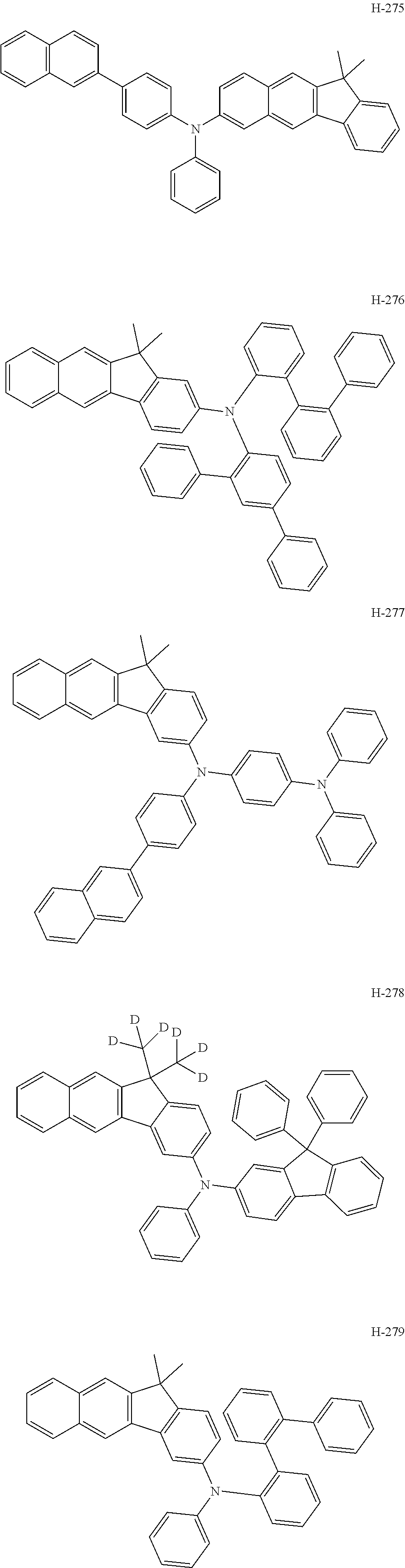 [SMILES: CC1(C)c2ccccc2-c2cc3cc(N(c4ccccc4)c4ccc(-c5ccc6ccccc6c5)cc4)ccc3cc21 ; CC1(C)c2cc(N(c3ccc(-c4ccccc4)cc3-c3ccccc3)c3ccccc3-c3ccccc3-c3ccccc3)ccc2-c2cc3ccccc3cc21 ; CC1(C)c2ccc(N(c3ccc(-c4ccc5ccccc5c4)cc3)c3ccc(N(c4ccccc4)c4ccccc4)cc3)cc2-c2cc3ccccc3cc21 ; [2H]C([2H])([2H])C1(C([2H])([2H])[2H])c2ccc(N(c3ccccc3)c3ccc4c(c3)C(c3ccccc3)(c3ccccc3)c3ccccc3-4)cc2-c2cc3ccccc3cc21 ; CC1(C)c2ccc(N(c3ccccc3)c3ccccc3-c3ccccc3-c3ccccc3)cc2-c2cc3ccccc3cc21]

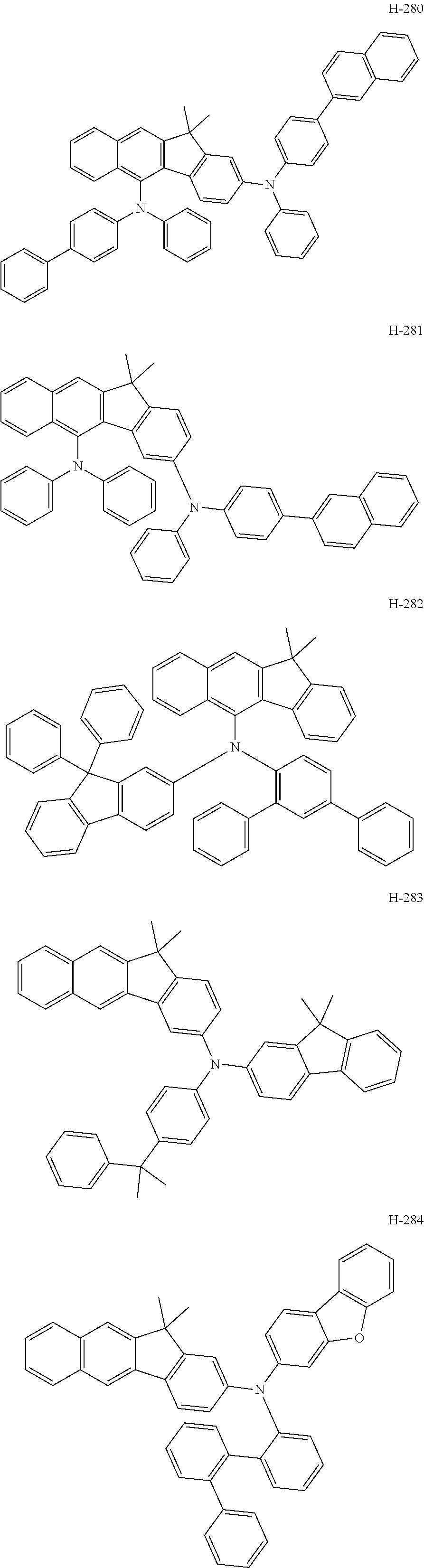 [SMILES: CC1(C)c2cc(N(c3ccccc3)c3ccc(-c4ccc5ccccc5c4)cc3)ccc2-c2c1cc1ccccc1c2N(c1ccccc1)c1ccc(-c2ccccc2)cc1 ; CC1(C)c2ccc(N(c3ccccc3)c3ccc(-c4ccc5ccccc5c4)cc3)cc2-c2c1cc1ccccc1c2N(c1ccccc1)c1ccccc1 ; CC1(C)c2ccccc2-c2c1cc1ccccc1c2N(c1ccc2c(c1)C(c1ccccc1)(c1ccccc1)c1ccccc1-2)c1ccc(-c2ccccc2)cc1-c1ccccc1 ; CC(C)(c1ccccc1)c1ccc(N(c2ccc3c(c2)-c2cc4ccccc4cc2C3(C)C)c2ccc3c(c2)C(C)(C)c2ccccc2-3)cc1 ; CC1(C)c2cc(N(c3ccc4c(c3)oc3ccccc34)c3ccccc3-c3ccccc3-c3ccccc3)ccc2-c2cc3ccccc3cc21]

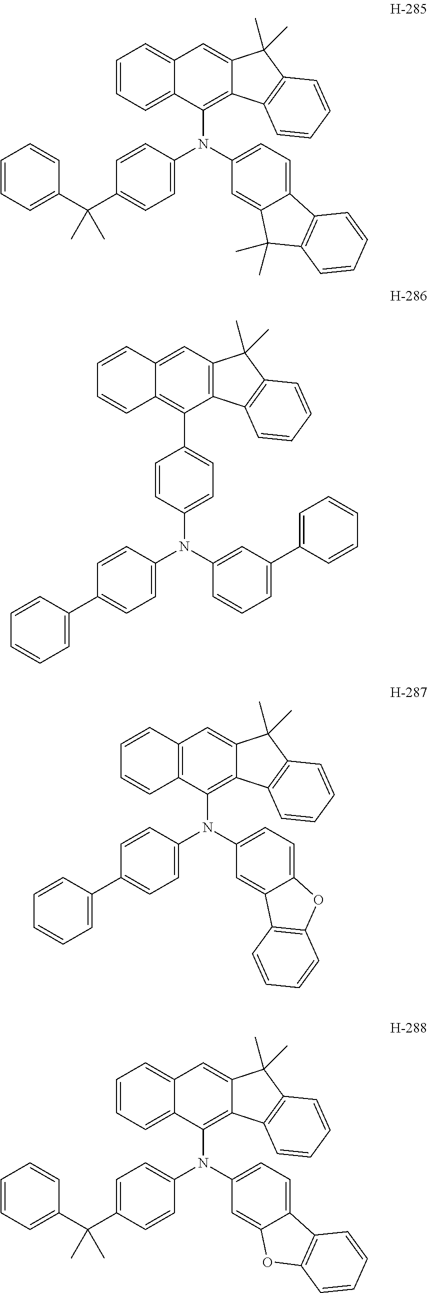 [SMILES: CC(C)(c1ccccc1)c1ccc(N(c2ccc3c(c2)C(C)(C)c2ccccc2-3)c2c3c(cc4ccccc24)C(C)(C)c2ccccc2-3)cc1 ; CC1(C)c2ccccc2-c2c1cc1ccccc1c2-c1ccc(N(c2ccc(-c3ccccc3)cc2)c2cccc(-c3ccccc3)c2)cc1 ; CC1(C)c2ccccc2-c2c1cc1ccccc1c2N(c1ccc(-c2ccccc2)cc1)c1ccc2oc3ccccc3c2c1 ; CC(C)(c1ccccc1)c1ccc(N(c2ccc3c(c2)oc2ccccc23)c2c3c(cc4ccccc24)C(C)(C)c2ccccc2-3)cc1]

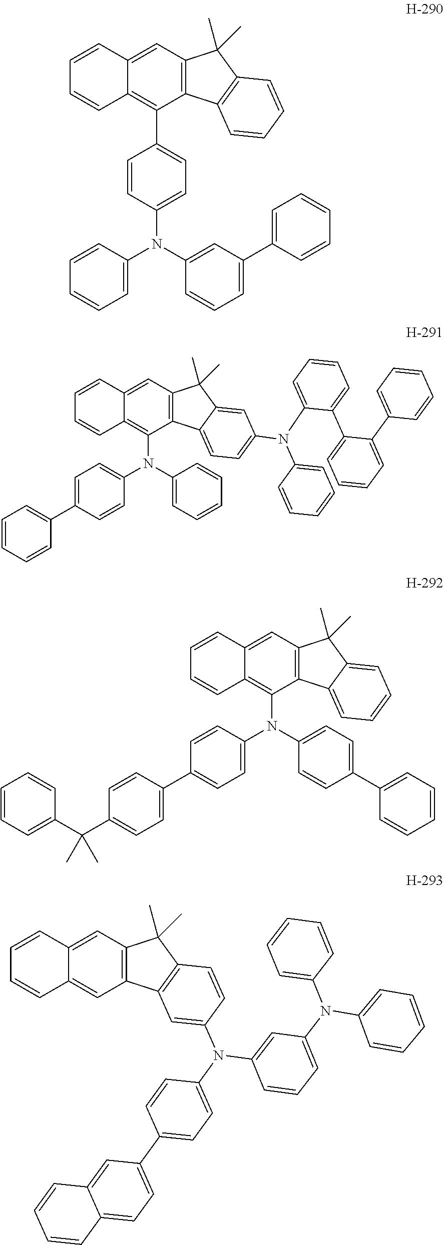 [SMILES: CC1(C)c2ccccc2-c2c1cc1ccccc1c2-c1ccc(N(c2ccccc2)c2cccc(-c3ccccc3)c2)cc1 ; CC1(C)c2cc(N(c3ccccc3)c3ccccc3-c3ccccc3-c3ccccc3)ccc2-c2c1cc1ccccc1c2N(c1ccccc1)c1ccc(-c2ccccc2)cc1 ; CC(C)(c1ccccc1)c1ccc(-c2ccc(N(c3ccc(-c4ccccc4)cc3)c3c4c(cc5ccccc35)C(C)(C)c3ccccc3-4)cc2)cc1 ; CC1(C)c2ccc(N(c3ccc(-c4ccc5ccccc5c4)cc3)c3cccc(N(c4ccccc4)c4ccccc4)c3)cc2-c2cc3ccccc3cc21]

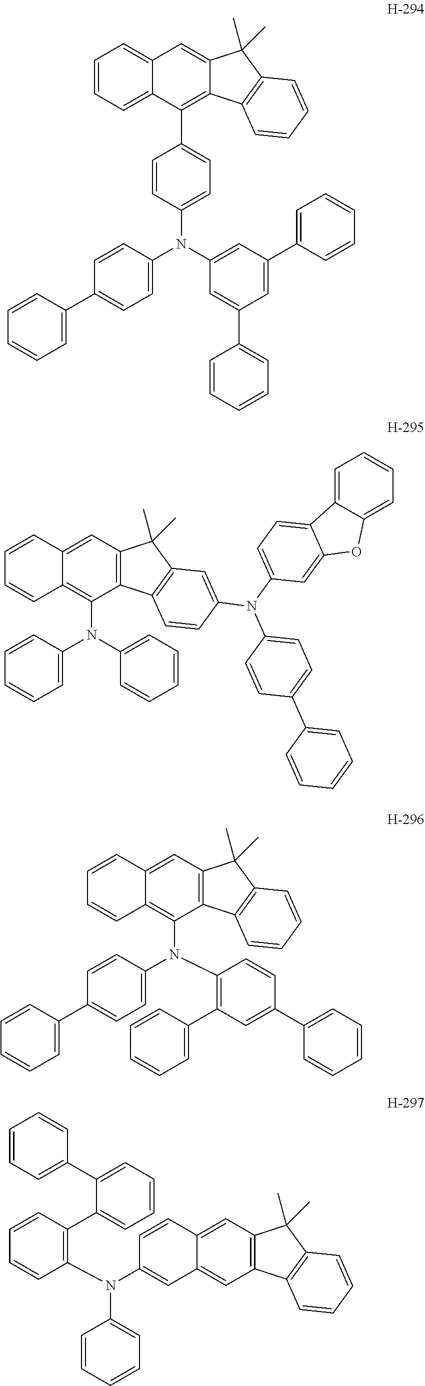

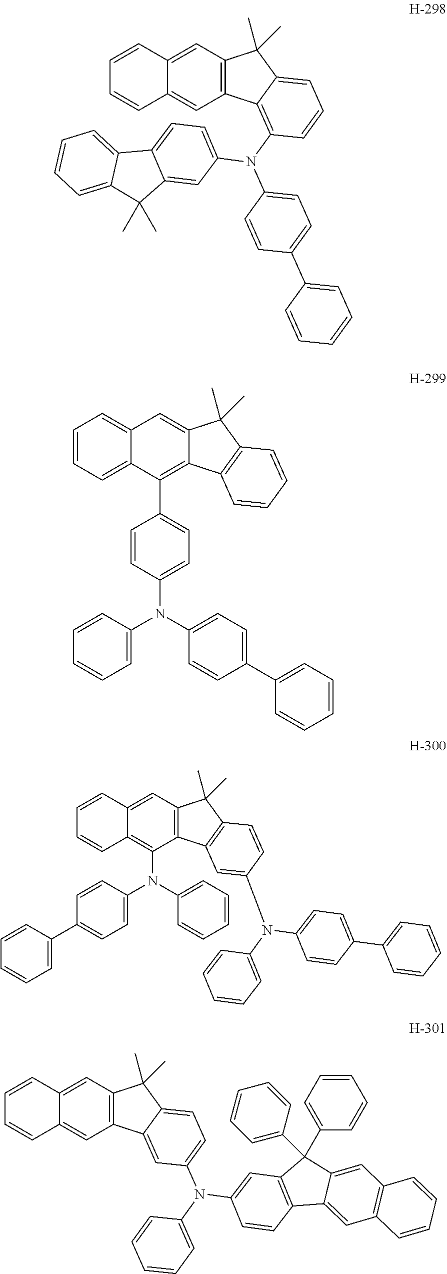 [SMILES: CC1(C)c2ccccc2-c2ccc(N(c3ccc(-c4ccccc4)cc3)c3cccc4c3-c3cc5ccccc5cc3C4(C)C)cc21 ; CC1(C)c2ccccc2-c2c1cc1ccccc1c2-c1ccc(N(c2ccccc2)c2ccc(-c3ccccc3)cc2)cc1 ; CC1(C)c2ccc(N(c3ccccc3)c3ccc(-c4ccccc4)cc3)cc2-c2c1cc1ccccc1c2N(c1ccccc1)c1ccc(-c2ccccc2)cc1 ; CC1(C)c2ccc(N(c3ccccc3)c3ccc4c(c3)C(c3ccccc3)(c3ccccc3)c3cc5ccccc5cc3-4)cc2-c2cc3ccccc3cc21]

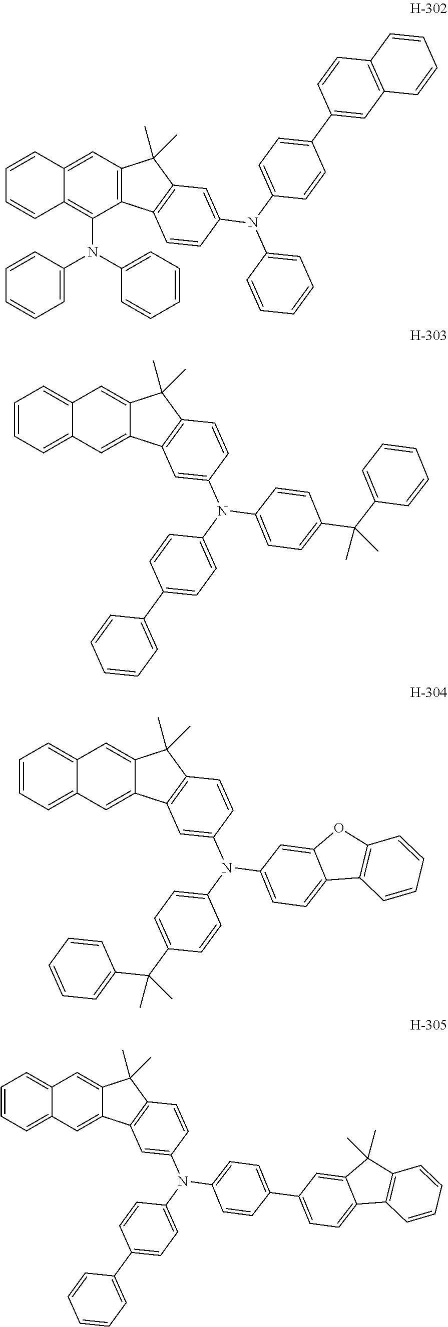

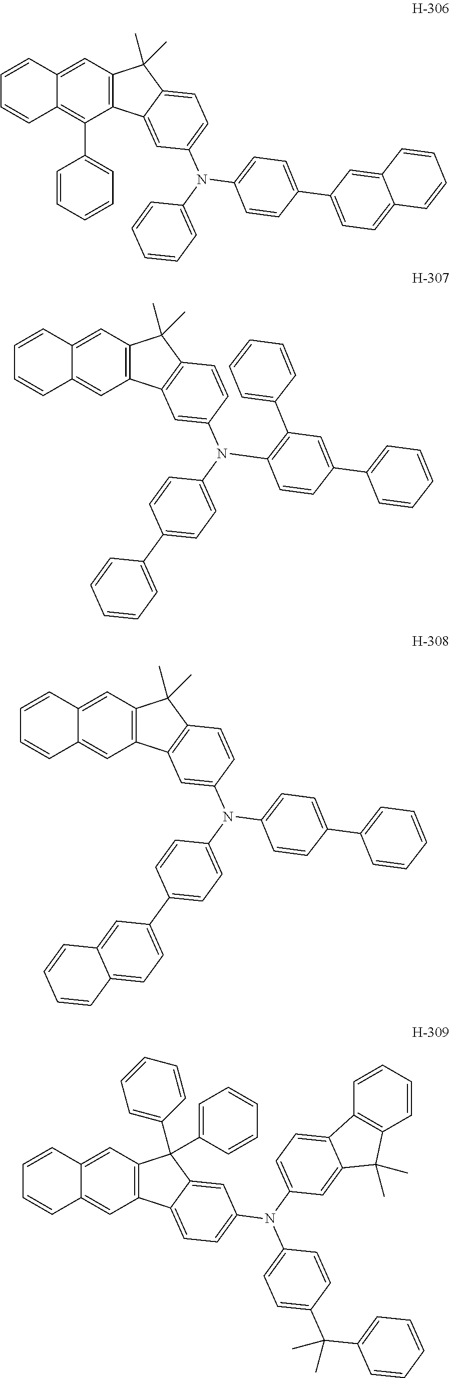

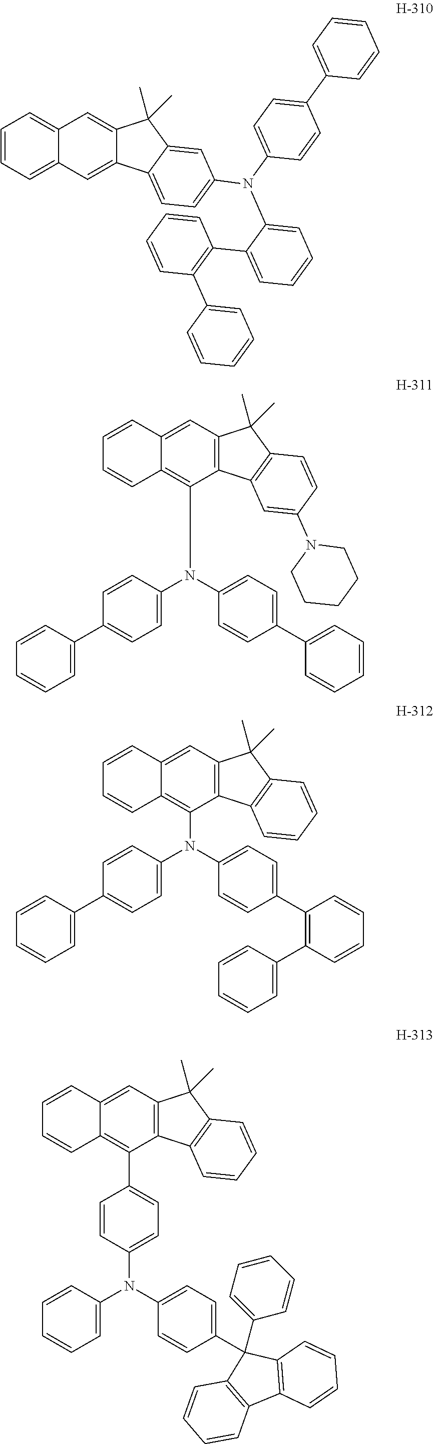

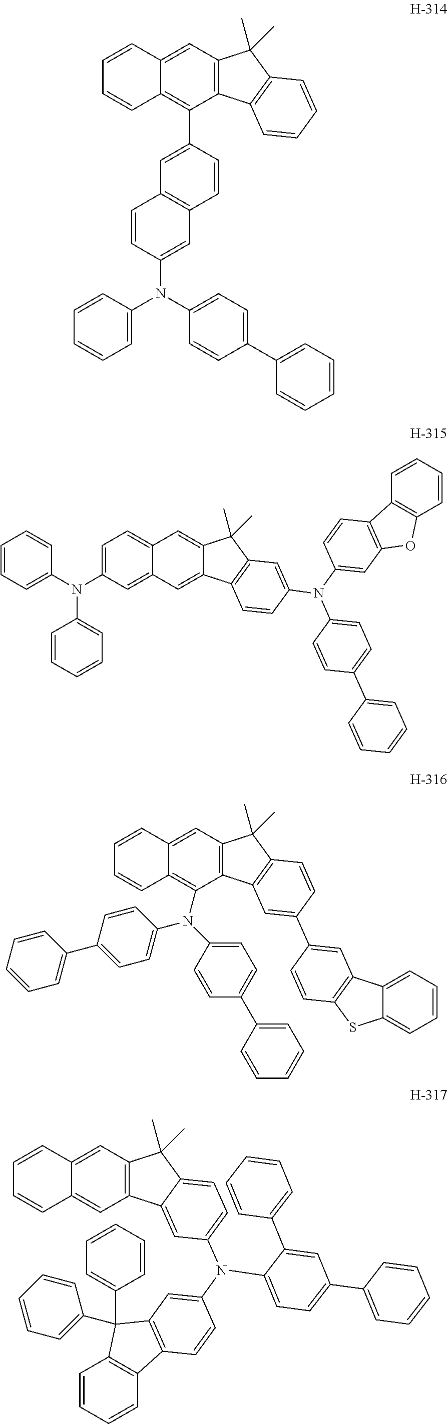 [SMILES: CC1(C)c2ccccc2-c2c1cc1ccccc1c2-c1ccc2cc(N(c3ccccc3)c3ccc(-c4ccccc4)cc3)ccc2c1 ; CC1(C)c2cc(N(c3ccc(-c4ccccc4)cc3)c3ccc4c(c3)oc3ccccc34)ccc2-c2cc3cc(N(c4ccccc4)c4ccccc4)ccc3cc21 ; CC1(C)c2ccc(-c3ccc4sc5ccccc5c4c3)cc2-c2c1cc1ccccc1c2N(c1ccc(-c2ccccc2)cc1)c1ccc(-c2ccccc2)cc1 ; CC1(C)c2ccc(N(c3ccc4c(c3)C(c3ccccc3)(c3ccccc3)c3ccccc3-4)c3ccc(-c4ccccc4)cc3-c3ccccc3)cc2-c2cc3ccccc3cc21]

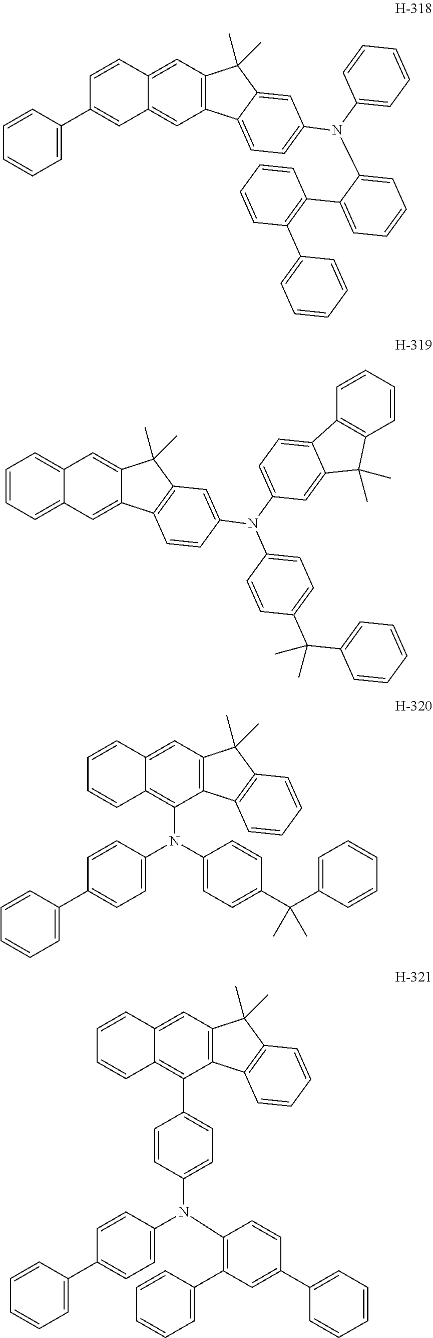 [SMILES: CC1(C)c2cc(N(c3ccccc3)c3ccccc3-c3ccccc3-c3ccccc3)ccc2-c2cc3cc(-c4ccccc4)ccc3cc21 ; CC(C)(c1ccccc1)c1ccc(N(c2ccc3c(c2)C(C)(C)c2ccccc2-3)c2ccc3c(c2)C(C)(C)c2cc4ccccc4cc2-3)cc1 ; CC(C)(c1ccccc1)c1ccc(N(c2ccc(-c3ccccc3)cc2)c2c3c(cc4ccccc24)C(C)(C)c2ccccc2-3)cc1 ; CC1(C)c2ccccc2-c2c1cc1ccccc1c2-c1ccc(N(c2ccc(-c3ccccc3)cc2)c2ccc(-c3ccccc3)cc2-c2ccccc2)cc1]

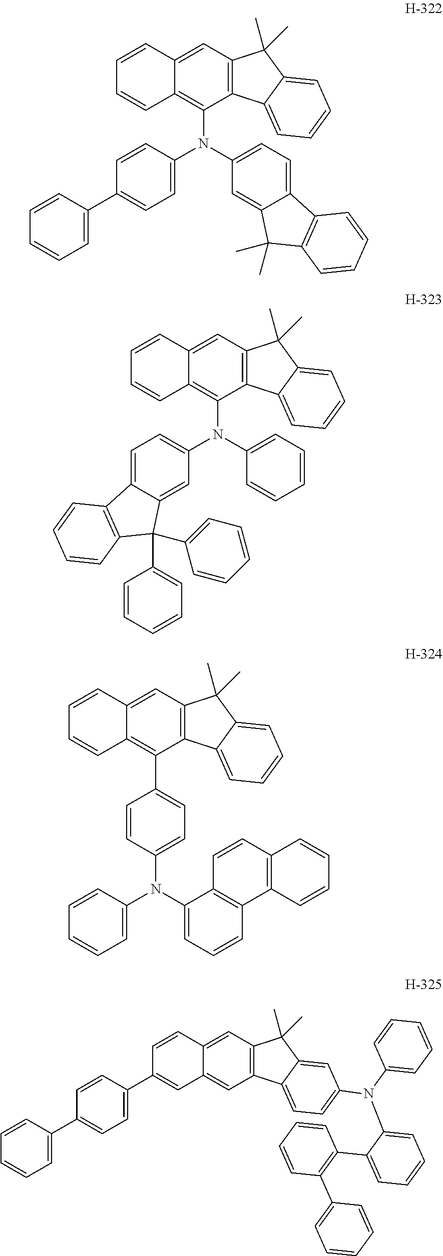

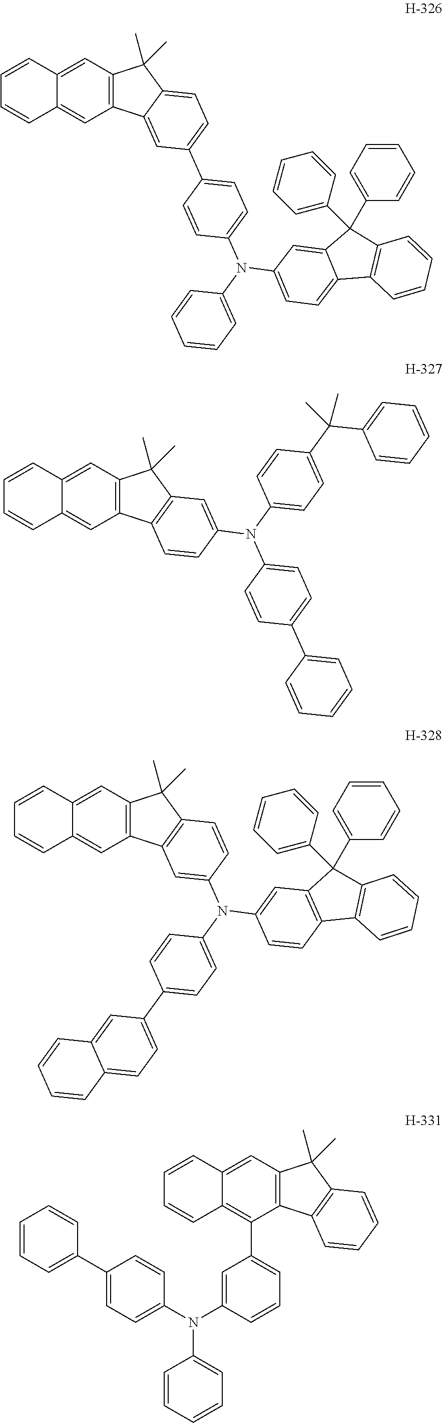 [SMILES: CC1(C)c2ccc(-c3ccc(N(c4ccccc4)c4ccc5c(c4)C(c4ccccc4)(c4ccccc4)c4ccccc4-5)cc3)cc2-c2cc3ccccc3cc21 ; CC(C)(c1ccccc1)c1ccc(N(c2ccc(-c3ccccc3)cc2)c2ccc3c(c2)C(C)(C)c2cc4ccccc4cc2-3)cc1 ; CC1(C)c2ccc(N(c3ccc(-c4ccc5ccccc5c4)cc3)c3ccc4c(c3)C(c3ccccc3)(c3ccccc3)c3ccccc3-4)cc2-c2cc3ccccc3cc21 ; CC1(C)c2ccccc2-c2c1cc1ccccc1c2-c1cccc(N(c2ccccc2)c2ccc(-c3ccccc3)cc2)c1]

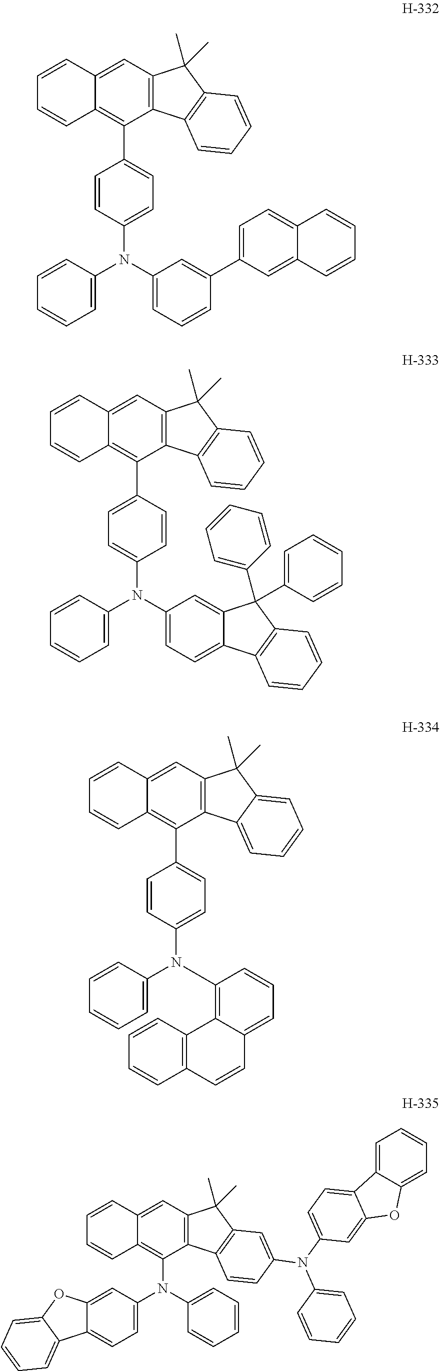

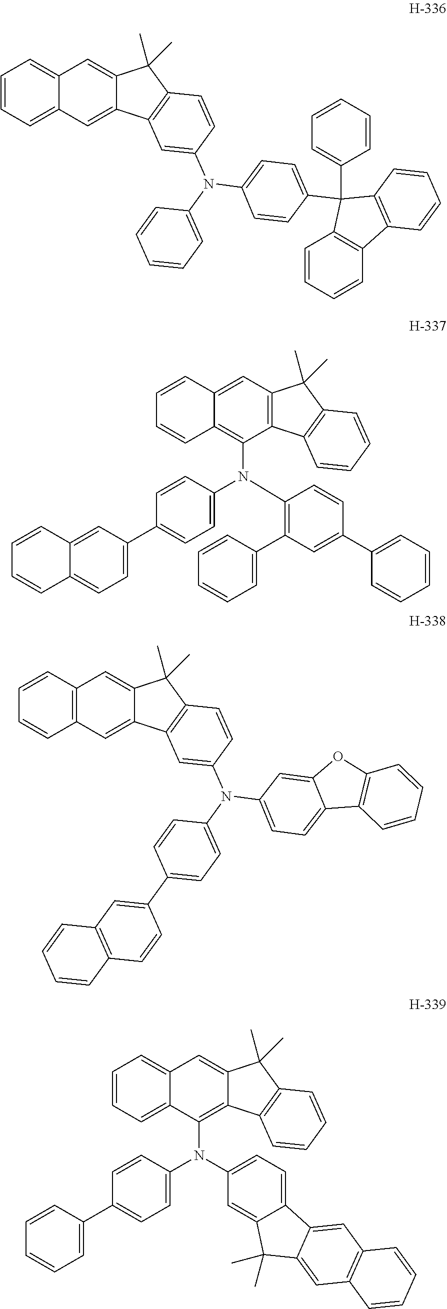 [SMILES: CC1(C)c2ccc(N(c3ccccc3)c3ccc(C4(c5ccccc5)c5ccccc5-c5ccccc54)cc3)cc2-c2cc3ccccc3cc21 ; CC1(C)c2ccccc2-c2c1cc1ccccc1c2N(c1ccc(-c2ccc3ccccc3c2)cc1)c1ccc(-c2ccccc2)cc1-c1ccccc1 ; CC1(C)c2ccc(N(c3ccc(-c4ccc5ccccc5c4)cc3)c3ccc4c(c3)oc3ccccc34)cc2-c2cc3ccccc3cc21 ; CC1(C)c2cc(N(c3ccc(-c4ccccc4)cc3)c3c4c(cc5ccccc35)C(C)(C)c3ccccc3-4)ccc2-c2cc3ccccc3cc21]

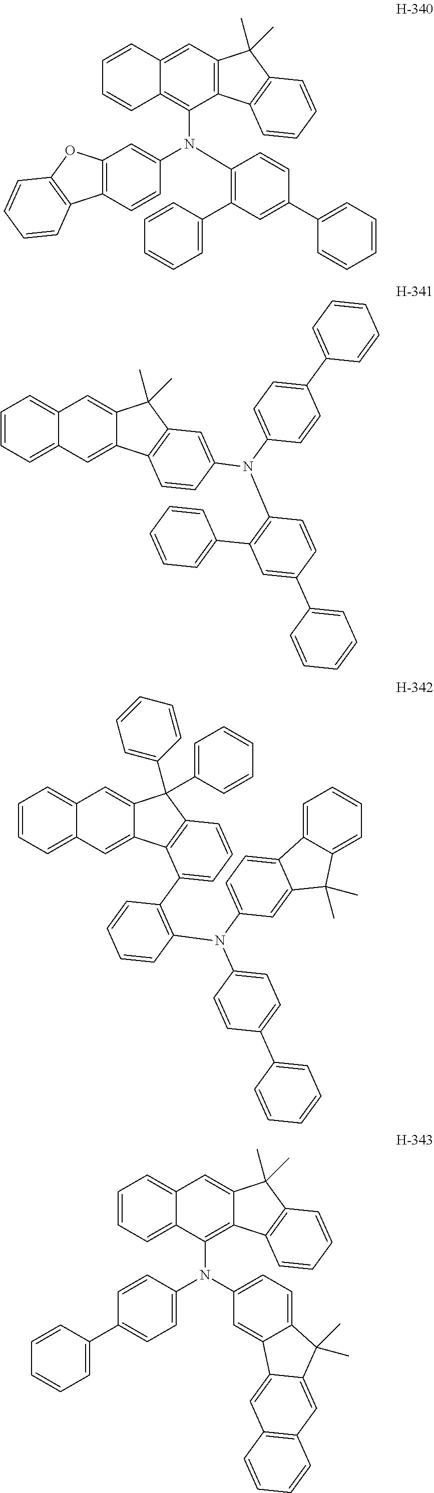

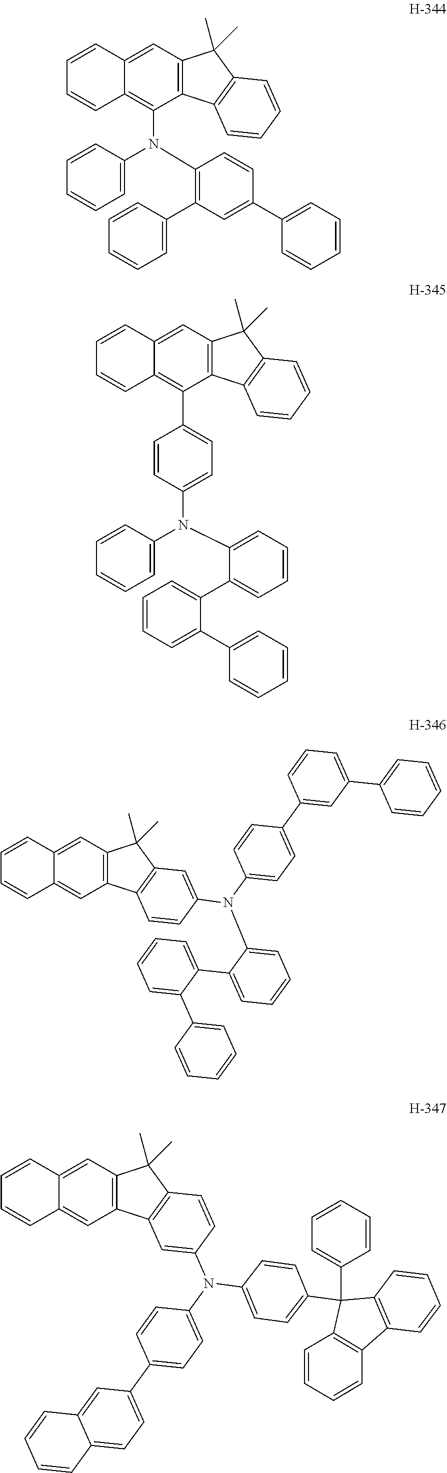 [SMILES: CC1(C)c2ccccc2-c2c1cc1ccccc1c2N(c1ccccc1)c1ccc(-c2ccccc2)cc1-c1ccccc1 ; CC1(C)c2ccccc2-c2c1cc1ccccc1c2-c1ccc(N(c2ccccc2)c2ccccc2-c2ccccc2-c2ccccc2)cc1 ; CC1(C)c2cc(N(c3ccc(-c4cccc(-c5ccccc5)c4)cc3)c3ccccc3-c3ccccc3-c3ccccc3)ccc2-c2cc3ccccc3cc21 ; CC1(C)c2ccc(N(c3ccc(-c4ccc5ccccc5c4)cc3)c3ccc(C4(c5ccccc5)c5ccccc5-c5ccccc54)cc3)cc2-c2cc3ccccc3cc21]

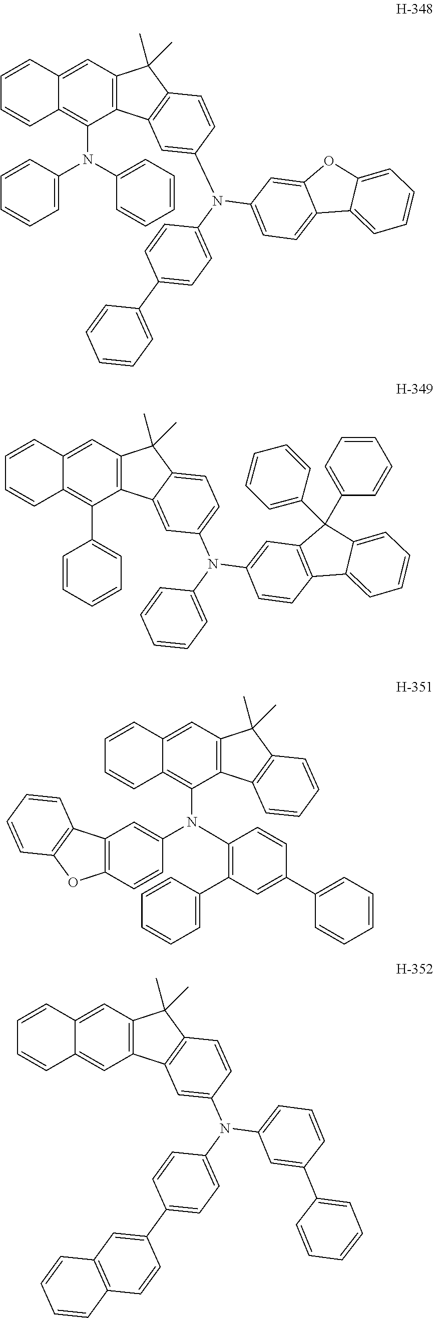

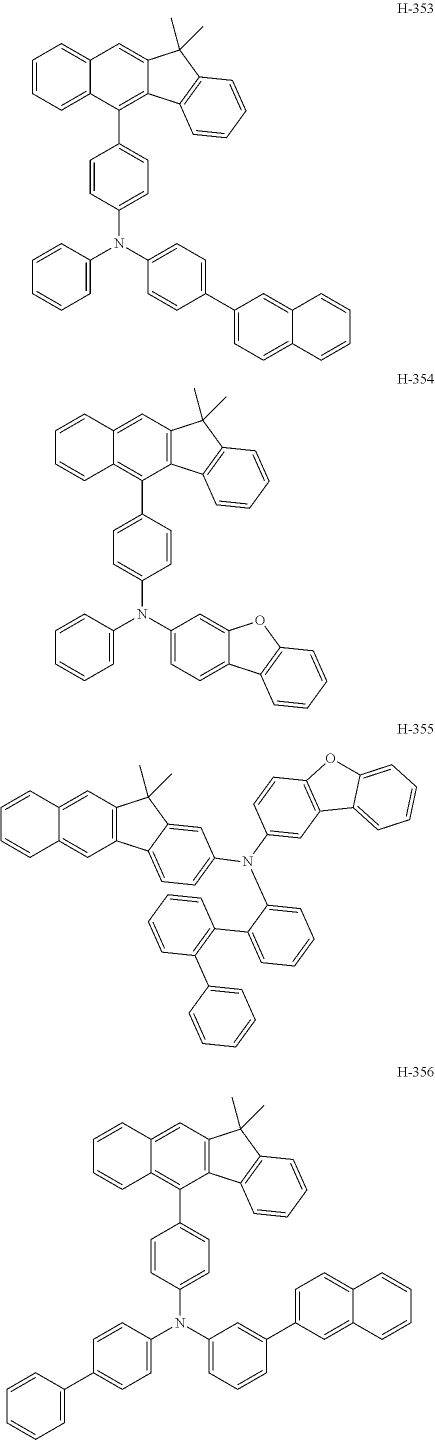 [SMILES: CC1(C)c2ccccc2-c2c1cc1ccccc1c2-c1ccc(N(c2ccccc2)c2ccc(-c3ccc4ccccc4c3)cc2)cc1 ; CC1(C)c2ccccc2-c2c1cc1ccccc1c2-c1ccc(N(c2ccccc2)c2ccc3c(c2)oc2ccccc23)cc1 ; CC1(C)c2cc(N(c3ccc4oc5ccccc5c4c3)c3ccccc3-c3ccccc3-c3ccccc3)ccc2-c2cc3ccccc3cc21 ; CC1(C)c2ccccc2-c2c1cc1ccccc1c2-c1ccc(N(c2ccc(-c3ccccc3)cc2)c2cccc(-c3ccc4ccccc4c3)c2)cc1]

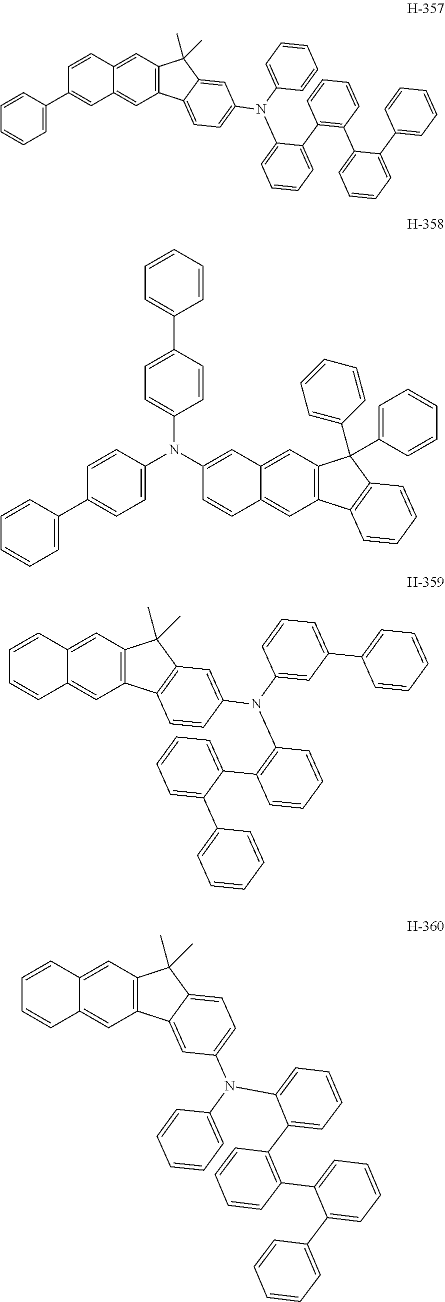 [SMILES: CC1(C)c2cc(N(c3ccccc3)c3ccccc3-c3ccccc3-c3ccccc3-c3ccccc3)ccc2-c2cc3cc(-c4ccccc4)ccc3cc21 ; c1ccc(-c2ccc(N(c3ccc(-c4ccccc4)cc3)c3ccc4cc5c(cc4c3)C(c3ccccc3)(c3ccccc3)c3ccccc3-5)cc2)cc1 ; CC1(C)c2cc(N(c3cccc(-c4ccccc4)c3)c3ccccc3-c3ccccc3-c3ccccc3)ccc2-c2cc3ccccc3cc21 ; CC1(C)c2ccc(N(c3ccccc3)c3ccccc3-c3ccccc3-c3ccccc3-c3ccccc3)cc2-c2cc3ccccc3cc21]

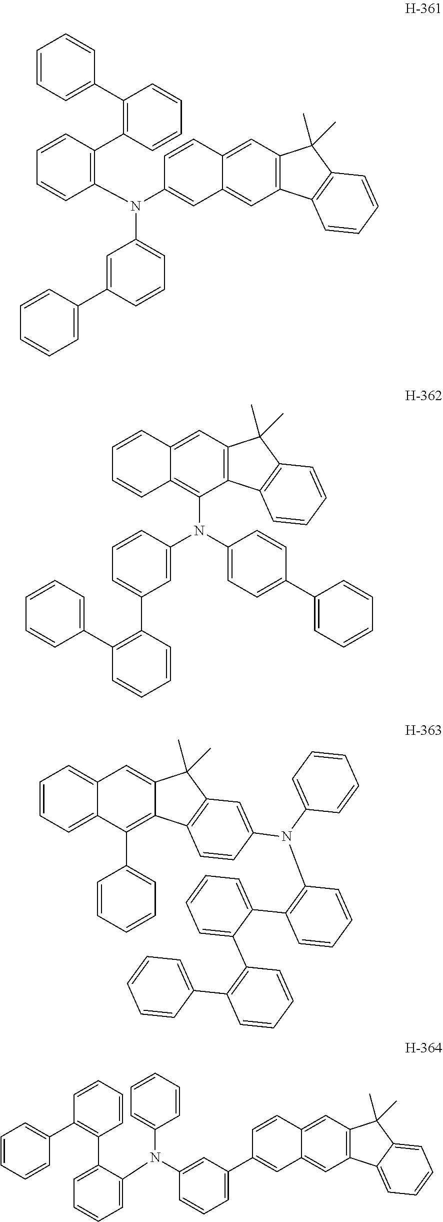 [SMILES: CC1(C)c2ccccc2-c2cc3cc(N(c4cccc(-c5ccccc5)c4)c4ccccc4-c4ccccc4-c4ccccc4)ccc3cc21 ; CC1(C)c2ccccc2-c2c1cc1ccccc1c2N(c1ccc(-c2ccccc2)cc1)c1cccc(-c2ccccc2-c2ccccc2)c1 ; CC1(C)c2cc(N(c3ccccc3)c3ccccc3-c3ccccc3-c3ccccc3-c3ccccc3)ccc2-c2c1cc1ccccc1c2-c1ccccc1 ; CC1(C)c2ccccc2-c2cc3cc(-c4cccc(N(c5ccccc5)c5ccccc5-c5ccccc5-c5ccccc5)c4)ccc3cc21]

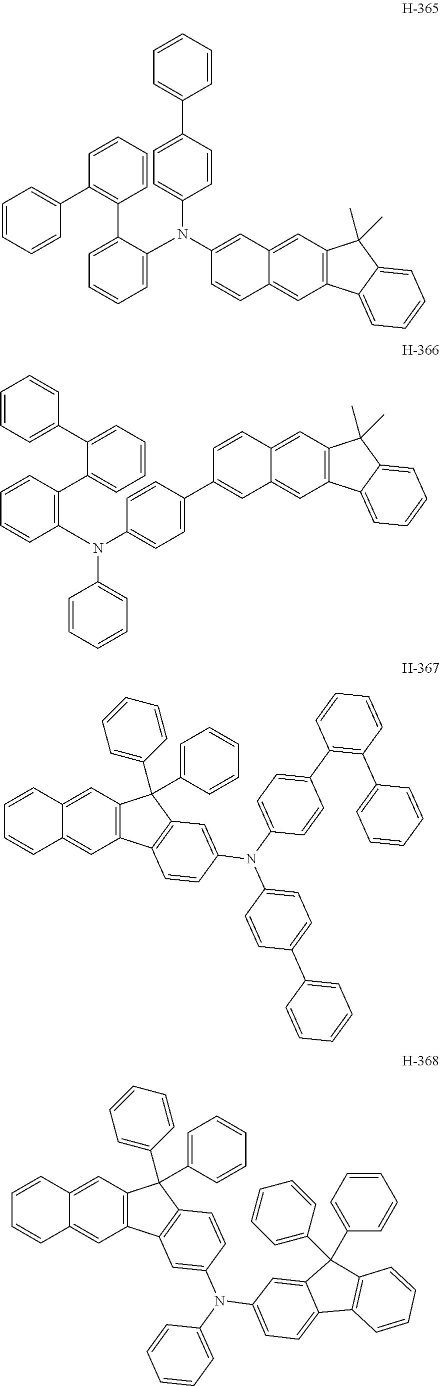 [SMILES: CC1(C)c2ccccc2-c2cc3ccc(N(c4ccc(-c5ccccc5)cc4)c4ccccc4-c4ccccc4-c4ccccc4)cc3cc21 ; CC1(C)c2ccccc2-c2cc3cc(-c4ccc(N(c5ccccc5)c5ccccc5-c5ccccc5-c5ccccc5)cc4)ccc3cc21 ; c1ccc(-c2ccc(N(c3ccc(-c4ccccc4-c4ccccc4)cc3)c3ccc4c(c3)C(c3ccccc3)(c3ccccc3)c3cc5ccccc5cc3-4)cc2)cc1 ; c1ccc(N(c2ccc3c(c2)-c2cc4ccccc4cc2C3(c2ccccc2)c2ccccc2)c2ccc3c(c2)C(c2ccccc2)(c2ccccc2)c2ccccc2-3)cc1]

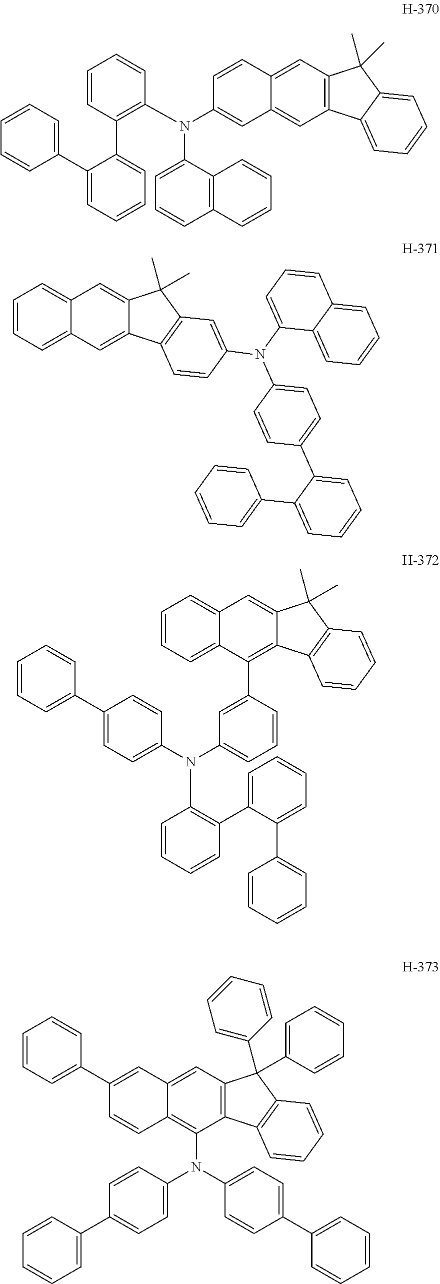

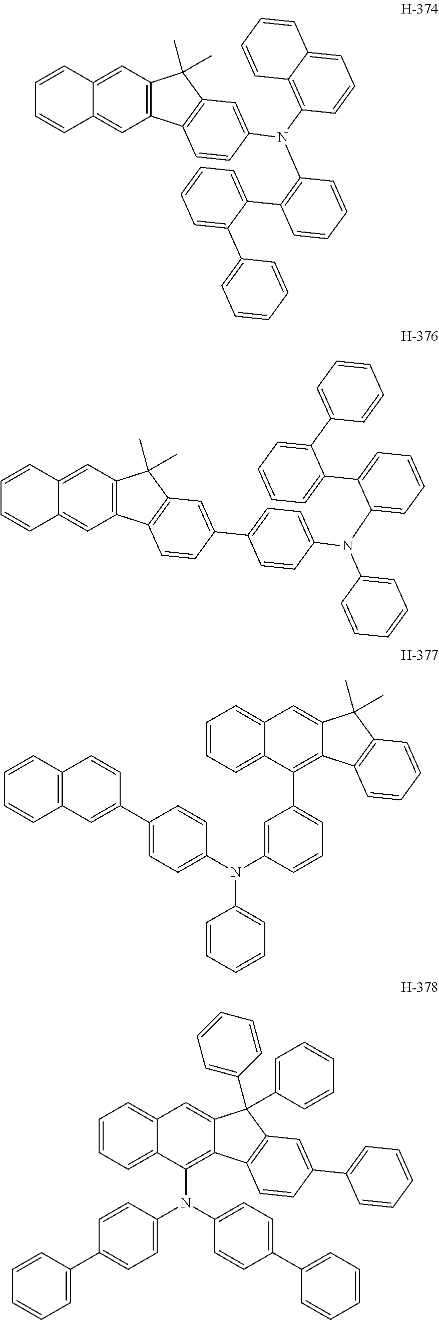

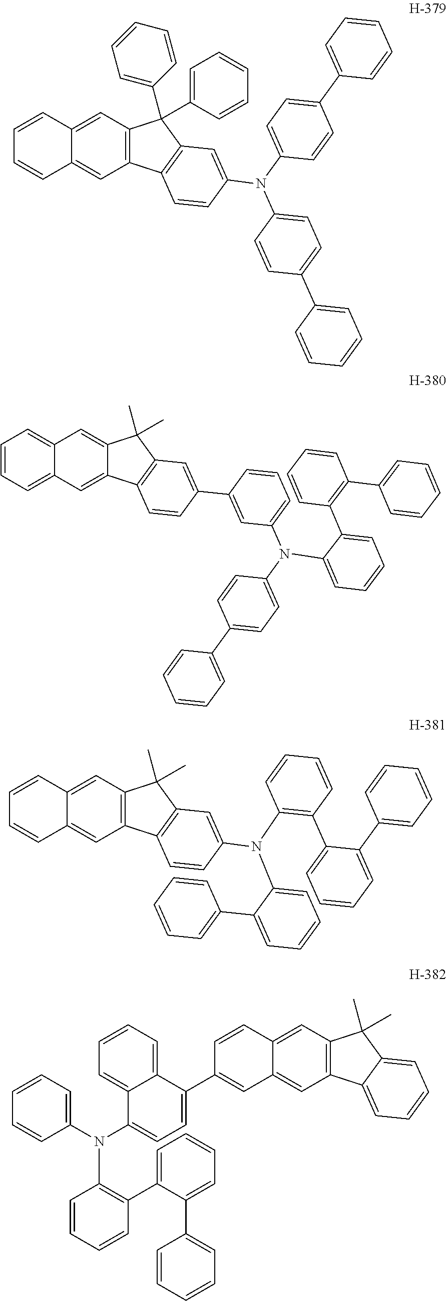 [SMILES: c1ccc(-c2ccc(N(c3ccc(-c4ccccc4)cc3)c3ccc4c(c3)C(c3ccccc3)(c3ccccc3)c3cc5ccccc5cc3-4)cc2)cc1 ; CC1(C)c2cc(-c3cccc(N(c4ccc(-c5ccccc5)cc4)c4ccccc4-c4ccccc4-c4ccccc4)c3)ccc2-c2cc3ccccc3cc21 ; CC1(C)c2cc(N(c3ccccc3-c3ccccc3)c3ccccc3-c3ccccc3-c3ccccc3)ccc2-c2cc3ccccc3cc21 ; CC1(C)c2ccccc2-c2cc3cc(-c4ccc(N(c5ccccc5)c5ccccc5-c5ccccc5-c5ccccc5)c5ccccc45)ccc3cc21]

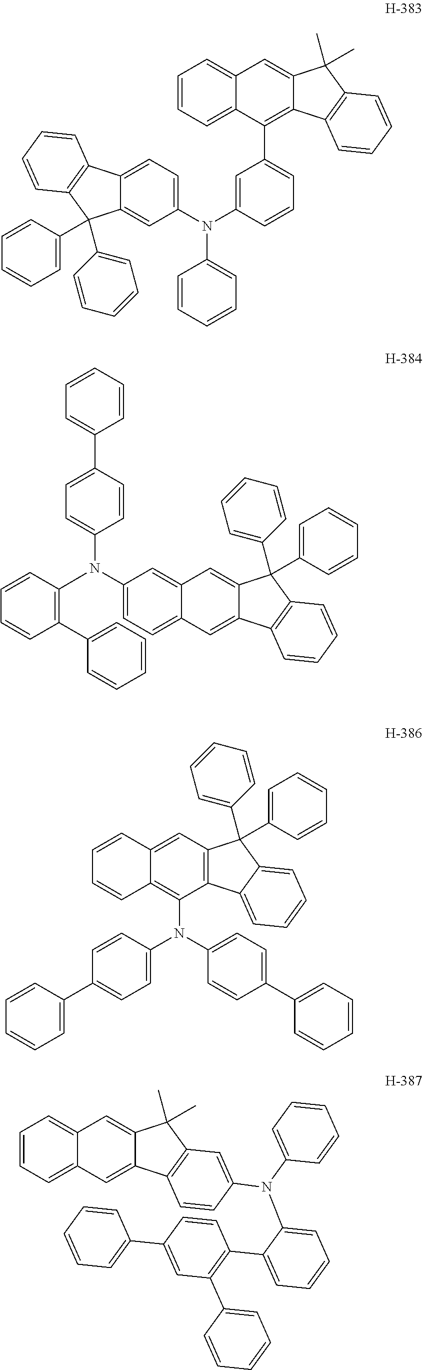 [SMILES: CC1(C)c2ccccc2-c2c1cc1ccccc1c2-c1cccc(N(c2ccccc2)c2ccc3c(c2)C(c2ccccc2)(c2ccccc2)c2ccccc2-3)c1 ; c1ccc(-c2ccc(N(c3ccc4cc5c(cc4c3)C(c3ccccc3)(c3ccccc3)c3ccccc3-5)c3ccccc3-c3ccccc3)cc2)cc1 ; c1ccc(-c2ccc(N(c3ccc(-c4ccccc4)cc3)c3c4c(cc5ccccc35)C(c3ccccc3)(c3ccccc3)c3ccccc3-4)cc2)cc1 ; CC1(C)c2cc(N(c3ccccc3)c3ccccc3-c3ccc(-c4ccccc4)cc3-c3ccccc3)ccc2-c2cc3ccccc3cc21]

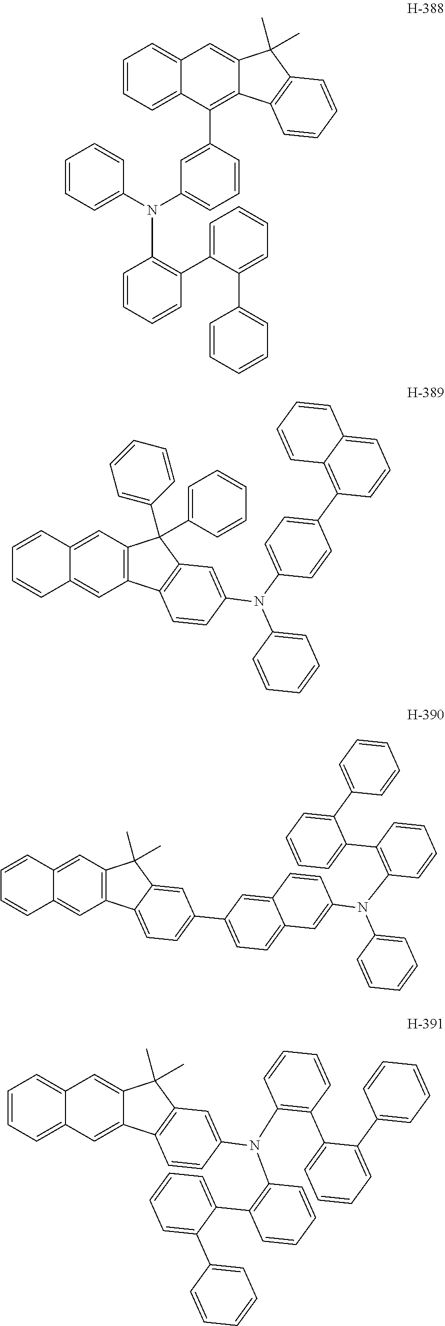 [SMILES: CC1(C)c2ccccc2-c2c1cc1ccccc1c2-c1cccc(N(c2ccccc2)c2ccccc2-c2ccccc2-c2ccccc2)c1 ; c1ccc(N(c2ccc(-c3cccc4ccccc34)cc2)c2ccc3c(c2)C(c2ccccc2)(c2ccccc2)c2cc4ccccc4cc2-3)cc1 ; CC1(C)c2cc(-c3ccc4cc(N(c5ccccc5)c5ccccc5-c5ccccc5-c5ccccc5)ccc4c3)ccc2-c2cc3ccccc3cc21 ; CC1(C)c2cc(N(c3ccccc3-c3ccccc3-c3ccccc3)c3ccccc3-c3ccccc3-c3ccccc3)ccc2-c2cc3ccccc3cc21]

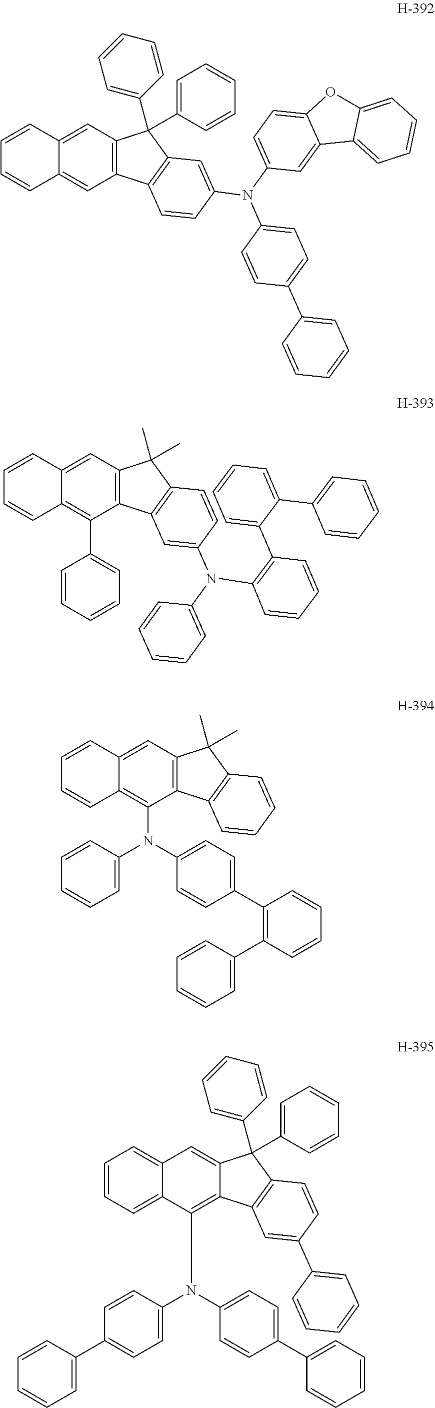 [SMILES: c1ccc(-c2ccc(N(c3ccc4c(c3)C(c3ccccc3)(c3ccccc3)c3cc5ccccc5cc3-4)c3ccc4oc5ccccc5c4c3)cc2)cc1 ; CC1(C)c2ccc(N(c3ccccc3)c3ccccc3-c3ccccc3-c3ccccc3)cc2-c2c1cc1ccccc1c2-c1ccccc1 ; CC1(C)c2ccccc2-c2c1cc1ccccc1c2N(c1ccccc1)c1ccc(-c2ccccc2-c2ccccc2)cc1 ; c1ccc(-c2ccc(N(c3ccc(-c4ccccc4)cc3)c3c4c(cc5ccccc35)C(c3ccccc3)(c3ccccc3)c3ccc(-c5ccccc5)cc3-4)cc2)cc1]

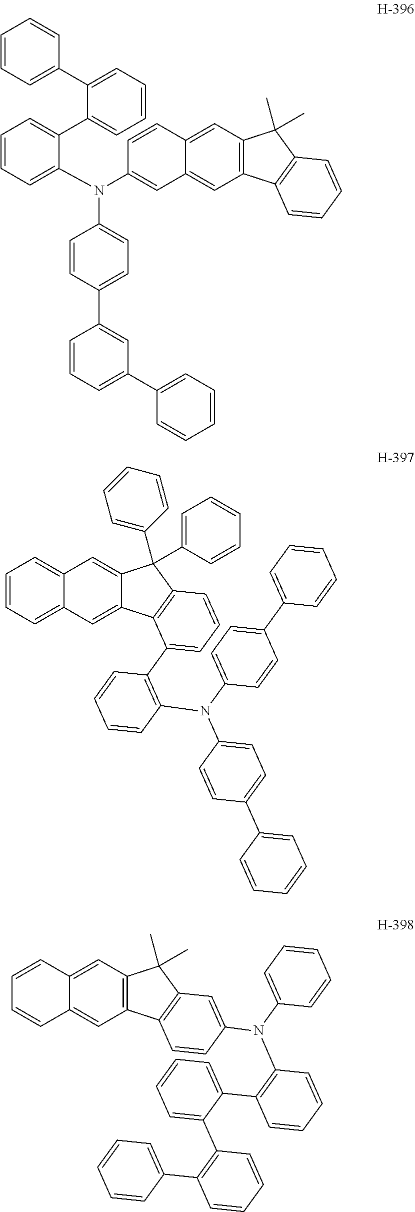

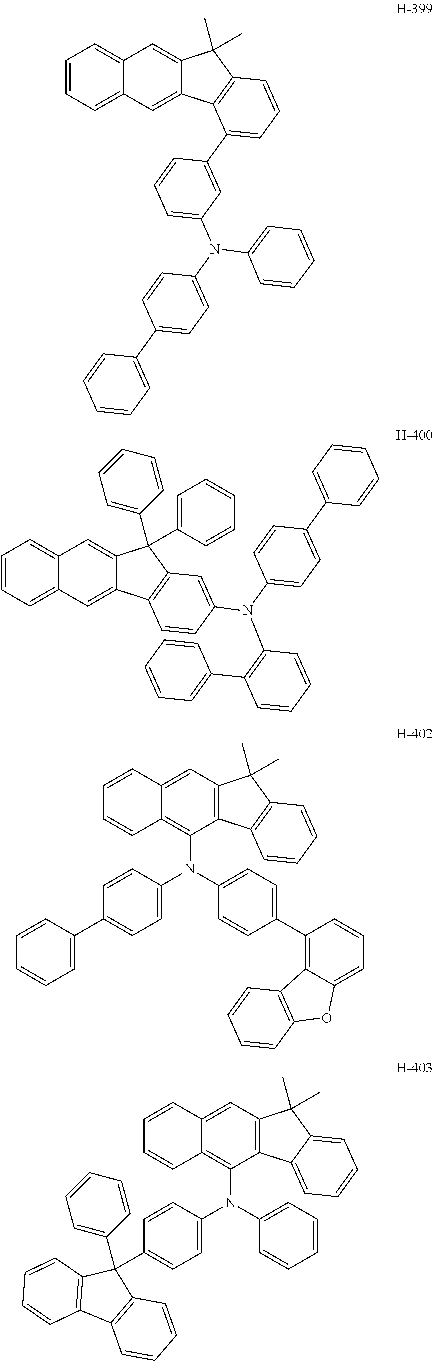

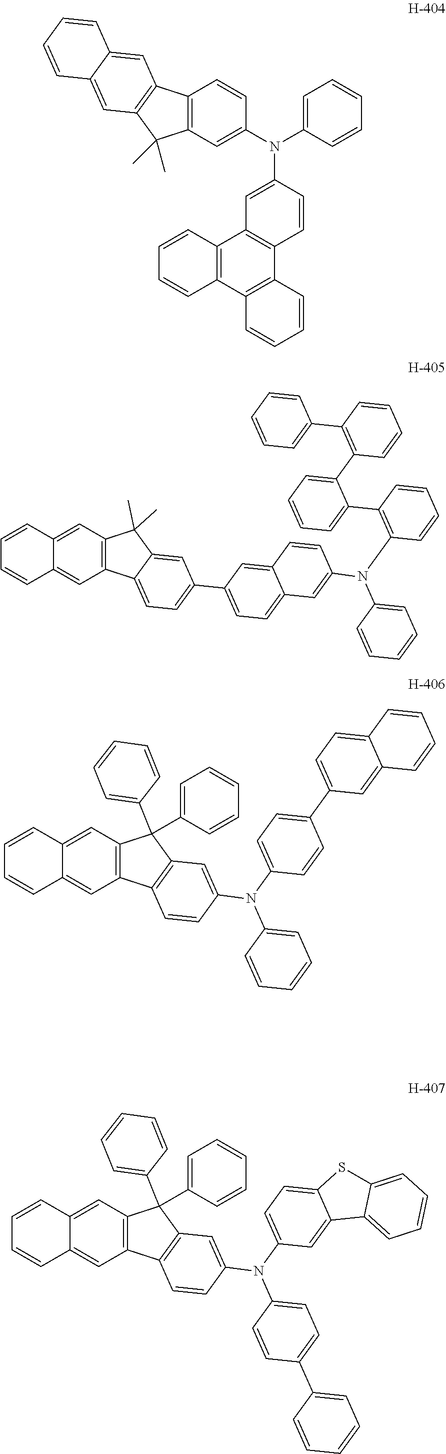 [SMILES: CC1(C)c2cc(N(c3ccccc3)c3ccc4c5ccccc5c5ccccc5c4c3)ccc2-c2cc3ccccc3cc21 ; CC1(C)c2cc(-c3ccc4cc(N(c5ccccc5)c5ccccc5-c5ccccc5-c5ccccc5-c5ccccc5)ccc4c3)ccc2-c2cc3ccccc3cc21 ; c1ccc(N(c2ccc(-c3ccc4ccccc4c3)cc2)c2ccc3c(c2)C(c2ccccc2)(c2ccccc2)c2cc4ccccc4cc2-3)cc1 ; c1ccc(-c2ccc(N(c3ccc4c(c3)C(c3ccccc3)(c3ccccc3)c3cc5ccccc5cc3-4)c3ccc4sc5ccccc5c4c3)cc2)cc1]

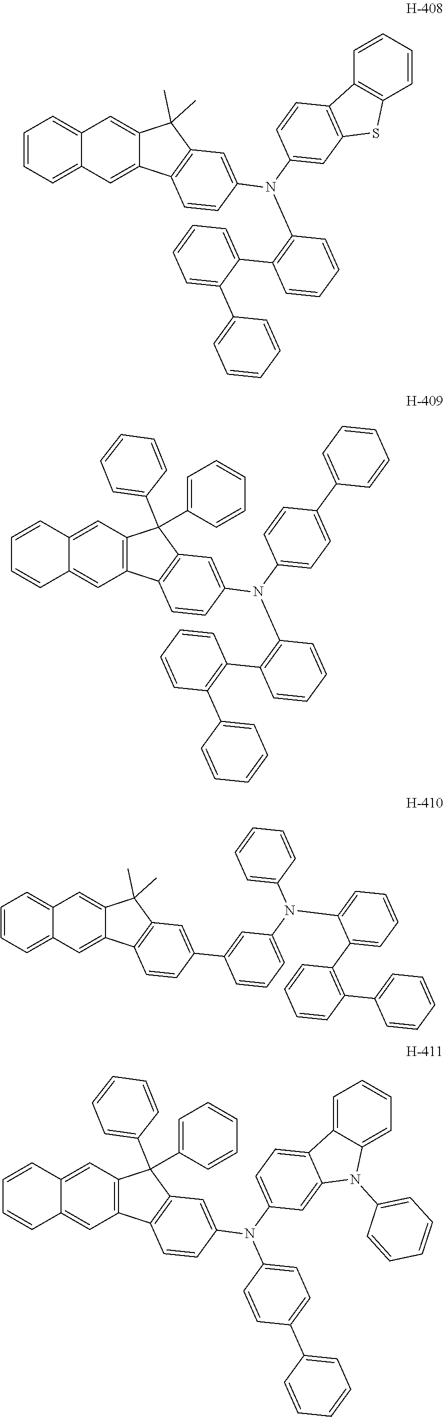

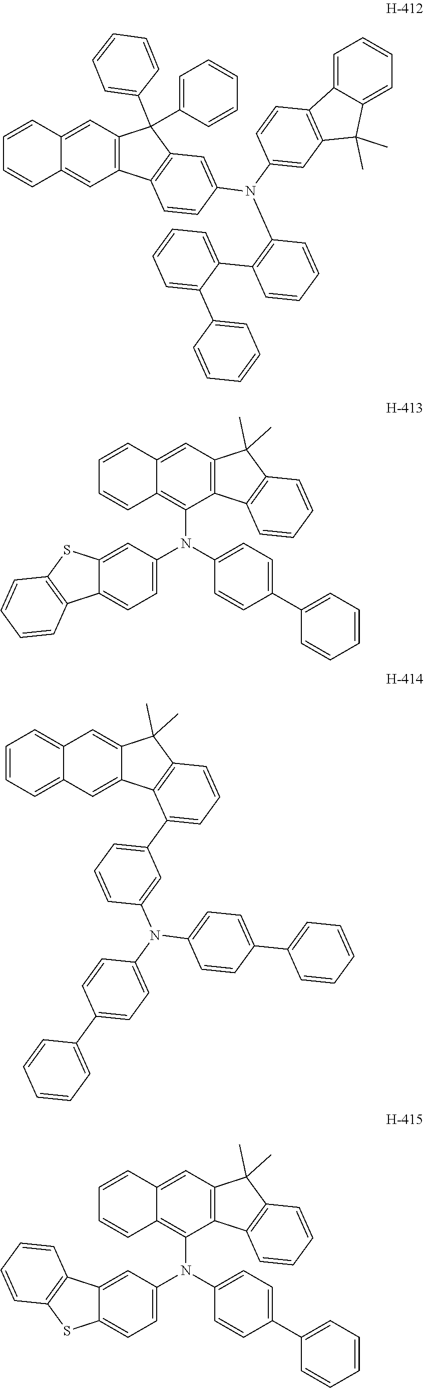

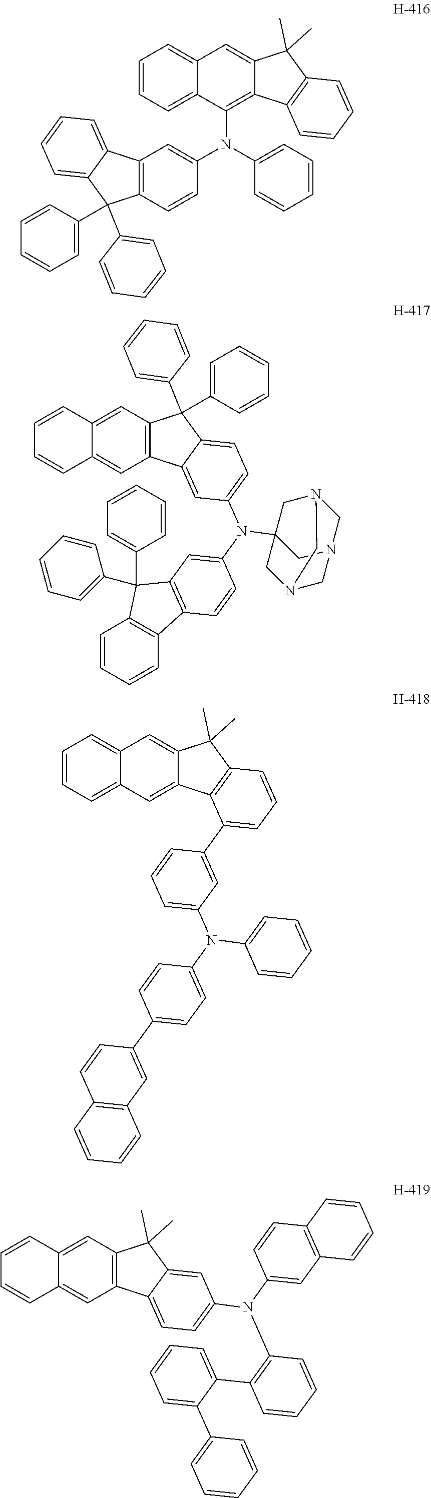

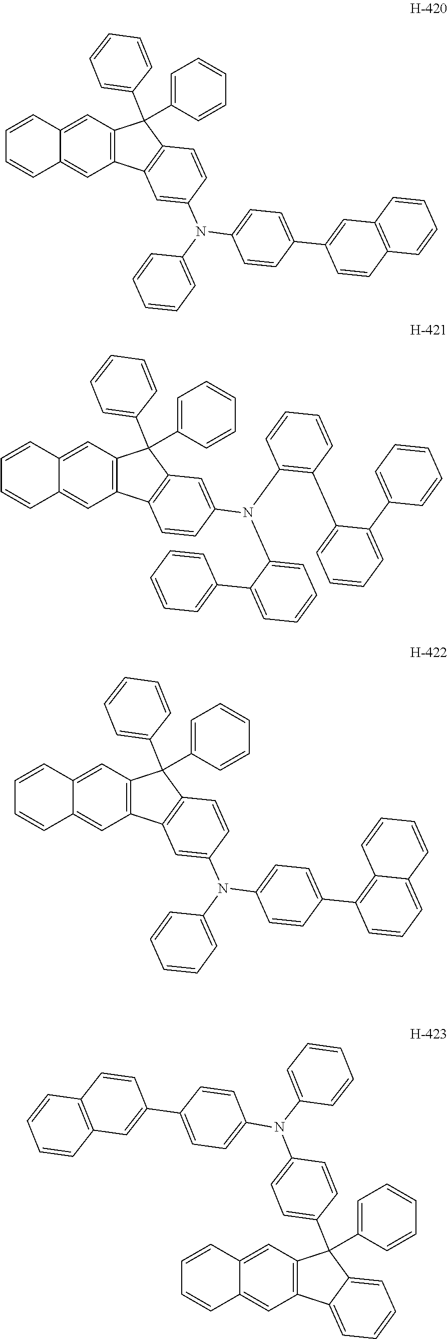 [SMILES: c1ccc(N(c2ccc(-c3ccc4ccccc4c3)cc2)c2ccc3c(c2)-c2cc4ccccc4cc2C3(c2ccccc2)c2ccccc2)cc1 ; c1ccc(-c2ccccc2-c2ccccc2N(c2ccc3c(c2)C(c2ccccc2)(c2ccccc2)c2cc4ccccc4cc2-3)c2ccccc2-c2ccccc2)cc1 ; c1ccc(N(c2ccc(-c3cccc4ccccc34)cc2)c2ccc3c(c2)-c2cc4ccccc4cc2C3(c2ccccc2)c2ccccc2)cc1 ; c1ccc(N(c2ccc(-c3ccc4ccccc4c3)cc2)c2ccc(C3(c4ccccc4)c4ccccc4-c4cc5ccccc5cc43)cc2)cc1]

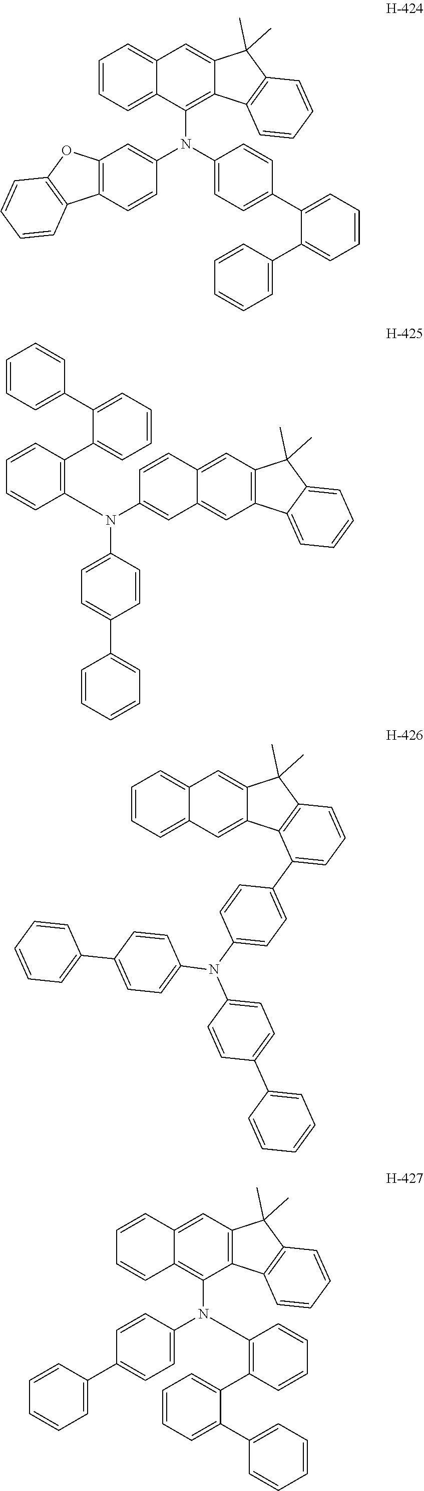

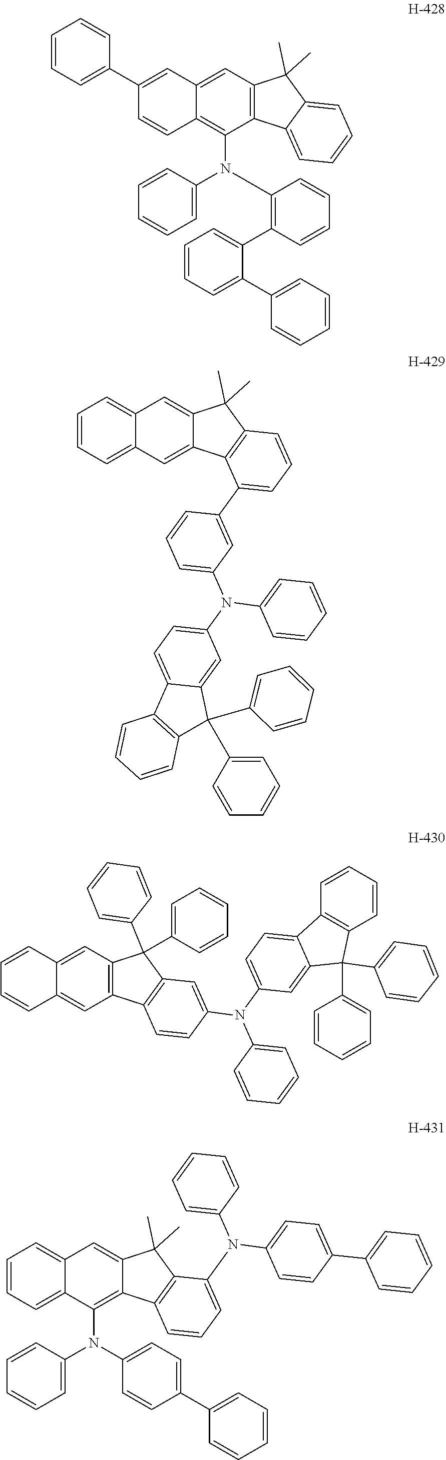 [SMILES: CC1(C)c2ccccc2-c2c1cc1cc(-c3ccccc3)ccc1c2N(c1ccccc1)c1ccccc1-c1ccccc1-c1ccccc1 ; CC1(C)c2cc3ccccc3cc2-c2c(-c3cccc(N(c4ccccc4)c4ccc5c(c4)C(c4ccccc4)(c4ccccc4)c4ccccc4-5)c3)cccc21 ; c1ccc(N(c2ccc3c(c2)C(c2ccccc2)(c2ccccc2)c2ccccc2-3)c2ccc3c(c2)C(c2ccccc2)(c2ccccc2)c2cc4ccccc4cc2-3)cc1 ; CC1(C)c2cc3ccccc3c(N(c3ccccc3)c3ccc(-c4ccccc4)cc3)c2-c2cccc(N(c3ccccc3)c3ccc(-c4ccccc4)cc3)c21]

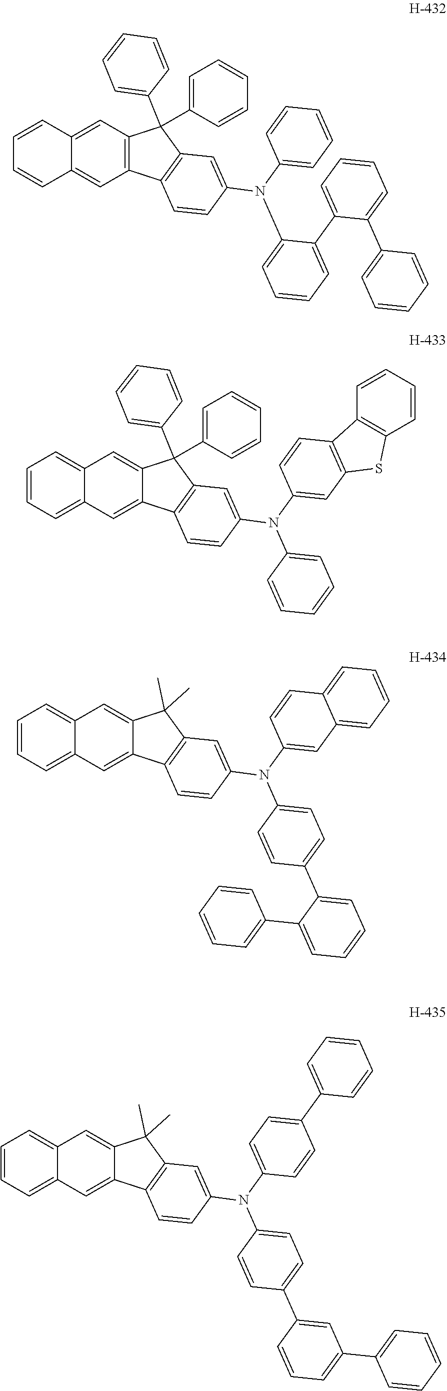 [SMILES: c1ccc(-c2ccccc2-c2ccccc2N(c2ccccc2)c2ccc3c(c2)C(c2ccccc2)(c2ccccc2)c2cc4ccccc4cc2-3)cc1 ; c1ccc(N(c2ccc3c(c2)C(c2ccccc2)(c2ccccc2)c2cc4ccccc4cc2-3)c2ccc3c(c2)sc2ccccc23)cc1 ; CC1(C)c2cc(N(c3ccc(-c4ccccc4-c4ccccc4)cc3)c3ccc4ccccc4c3)ccc2-c2cc3ccccc3cc21 ; CC1(C)c2cc(N(c3ccc(-c4ccccc4)cc3)c3ccc(-c4cccc(-c5ccccc5)c4)cc3)ccc2-c2cc3ccccc3cc21]

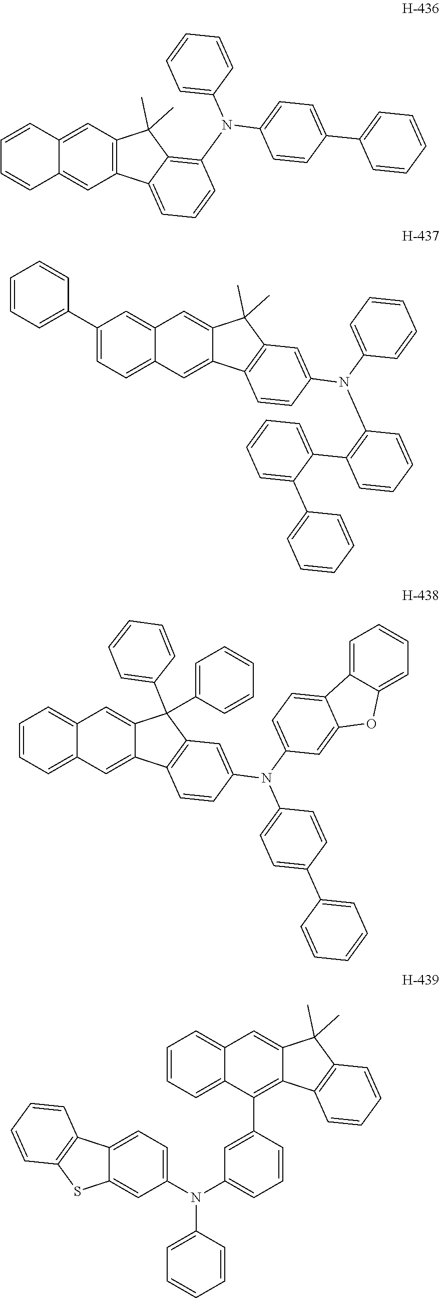 [SMILES: CC1(C)c2cc3ccccc3cc2-c2cccc(N(c3ccccc3)c3ccc(-c4ccccc4)cc3)c21 ; CC1(C)c2cc(N(c3ccccc3)c3ccccc3-c3ccccc3-c3ccccc3)ccc2-c2cc3ccc(-c4ccccc4)cc3cc21 ; c1ccc(-c2ccc(N(c3ccc4c(c3)C(c3ccccc3)(c3ccccc3)c3cc5ccccc5cc3-4)c3ccc4c(c3)oc3ccccc34)cc2)cc1 ; CC1(C)c2ccccc2-c2c1cc1ccccc1c2-c1cccc(N(c2ccccc2)c2ccc3c(c2)sc2ccccc23)c1]

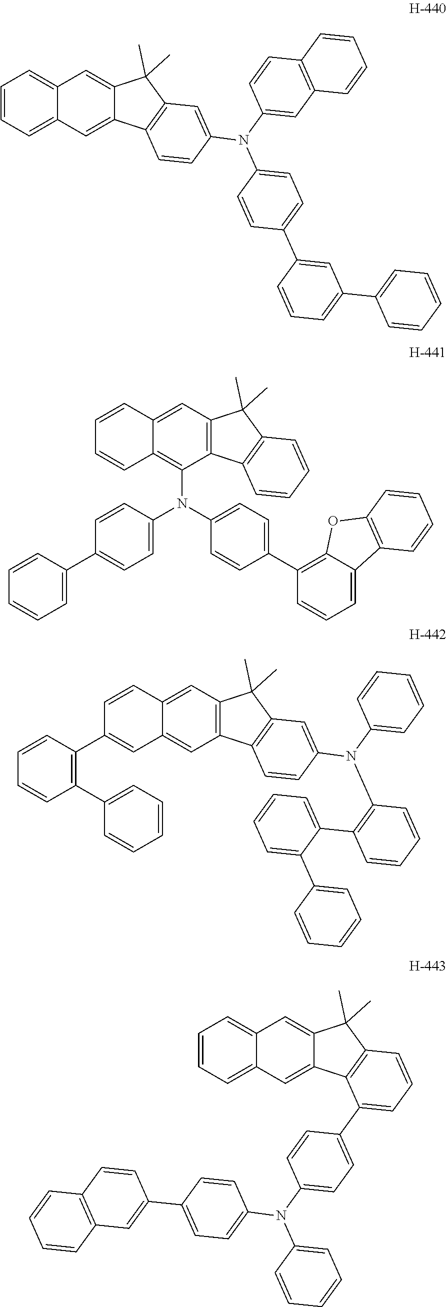

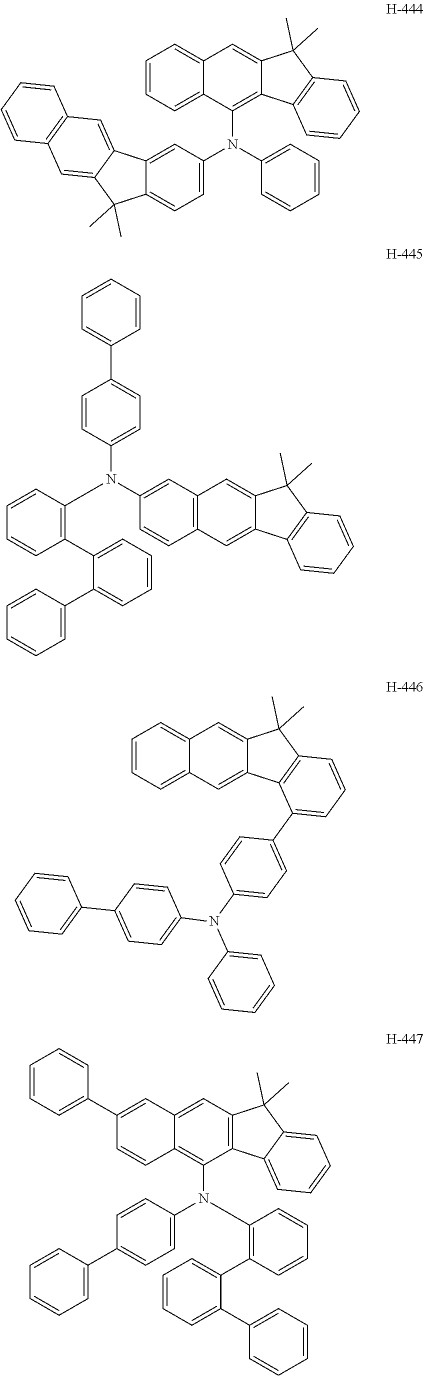

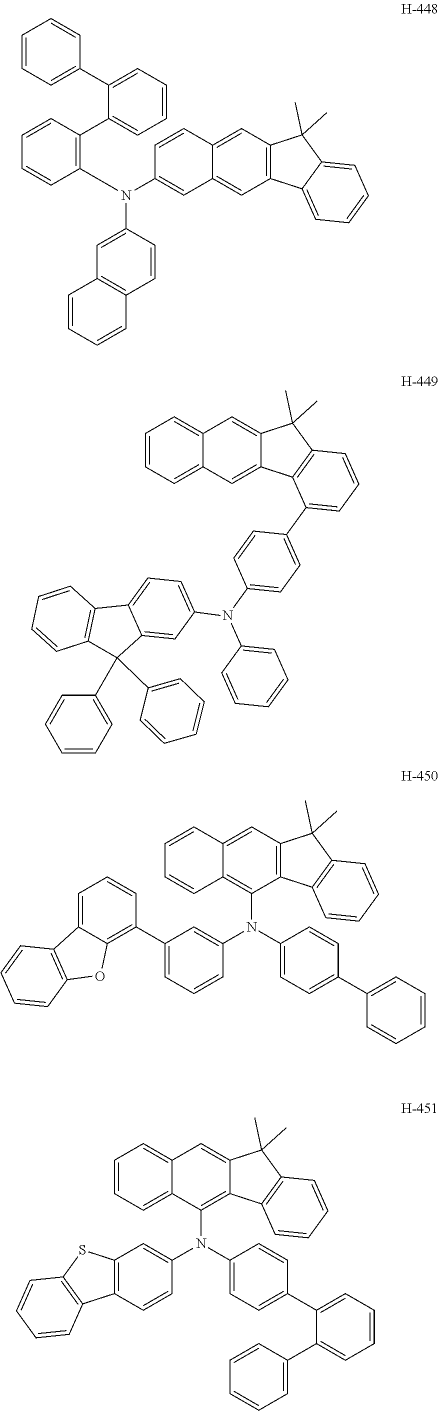 [SMILES: CC1(C)c2ccccc2-c2cc3cc(N(c4ccc5ccccc5c4)c4ccccc4-c4ccccc4-c4ccccc4)ccc3cc21 ; CC1(C)c2cc3ccccc3cc2-c2c(-c3ccc(N(c4ccccc4)c4ccc5c(c4)C(c4ccccc4)(c4ccccc4)c4ccccc4-5)cc3)cccc21 ; CC1(C)c2ccccc2-c2c1cc1ccccc1c2N(c1ccc(-c2ccccc2)cc1)c1cccc(-c2cccc3c2oc2ccccc23)c1 ; CC1(C)c2ccccc2-c2c1cc1ccccc1c2N(c1ccc(-c2ccccc2-c2ccccc2)cc1)c1ccc2c(c1)sc1ccccc12]

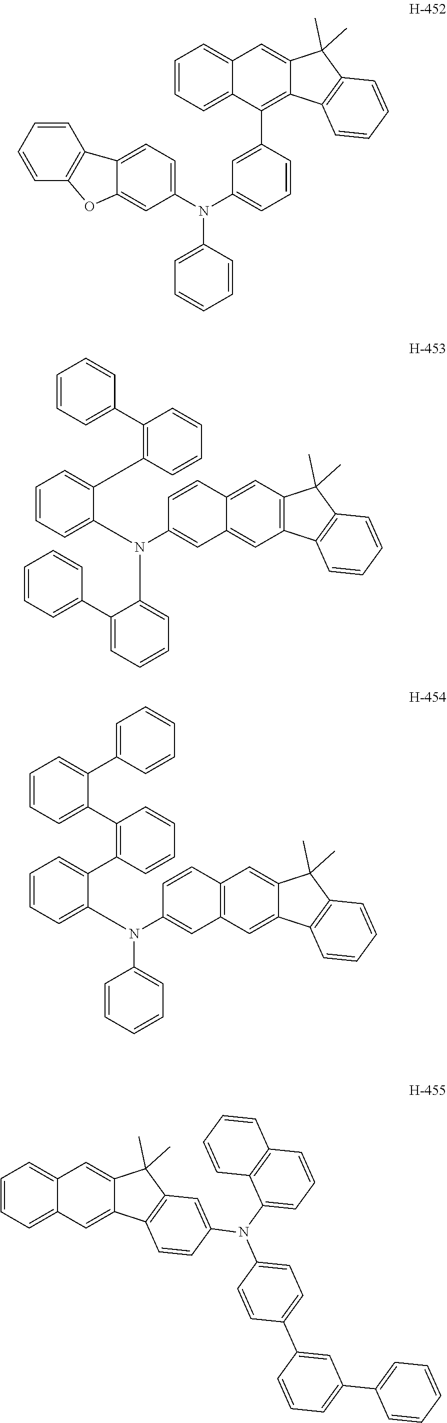

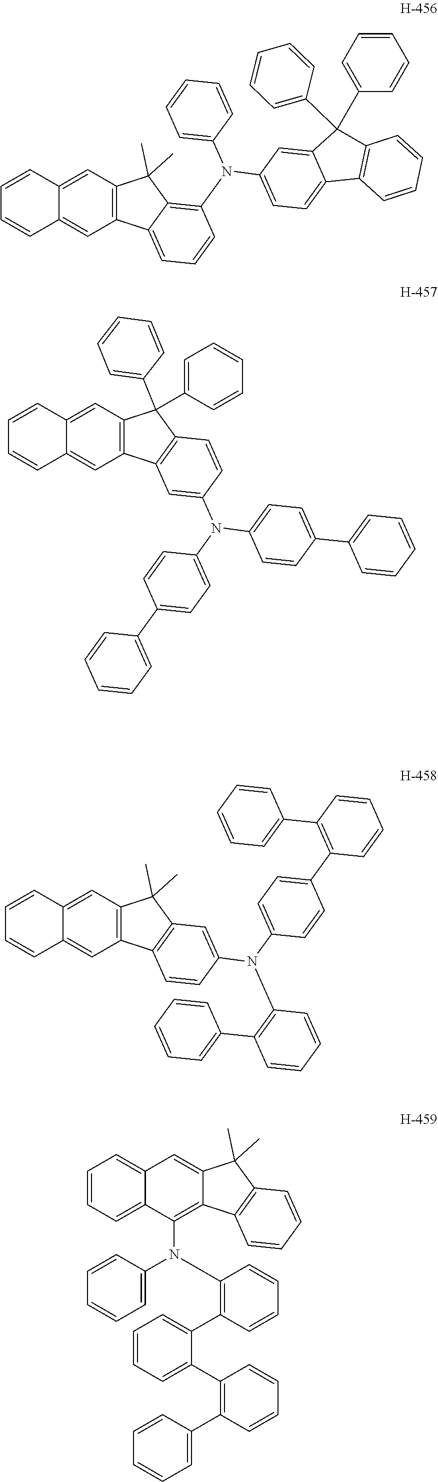 [SMILES: CC1(C)c2cc3ccccc3cc2-c2cccc(N(c3ccccc3)c3ccc4c(c3)C(c3ccccc3)(c3ccccc3)c3ccccc3-4)c21 ; c1ccc(-c2ccc(N(c3ccc(-c4ccccc4)cc3)c3ccc4c(c3)-c3cc5ccccc5cc3C4(c3ccccc3)c3ccccc3)cc2)cc1 ; CC1(C)c2cc(N(c3ccc(-c4ccccc4-c4ccccc4)cc3)c3ccccc3-c3ccccc3)ccc2-c2cc3ccccc3cc21 ; CC1(C)c2ccccc2-c2c1cc1ccccc1c2N(c1ccccc1)c1ccccc1-c1ccccc1-c1ccccc1-c1ccccc1]

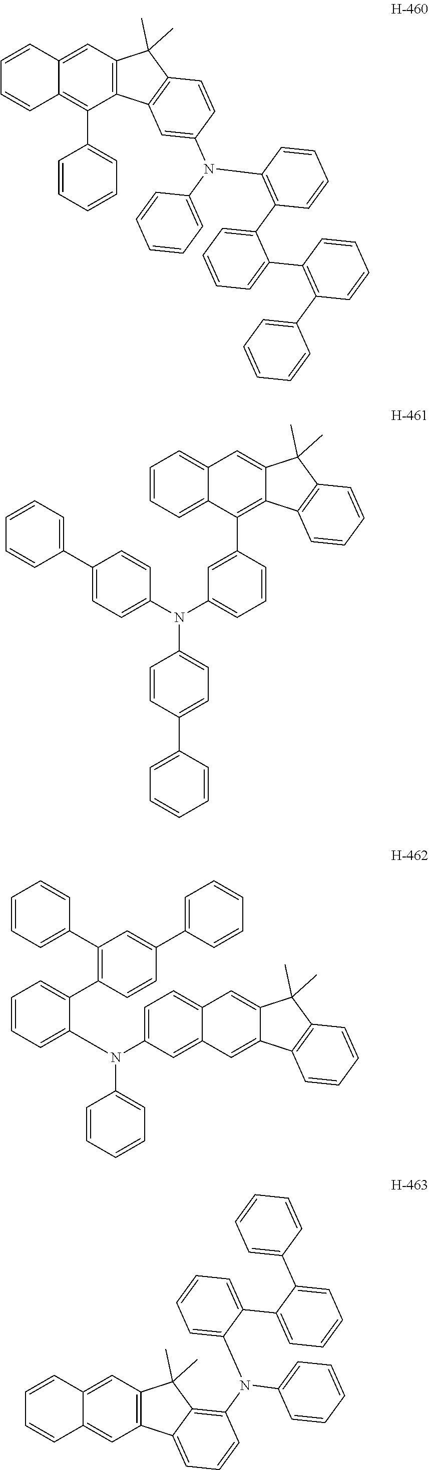

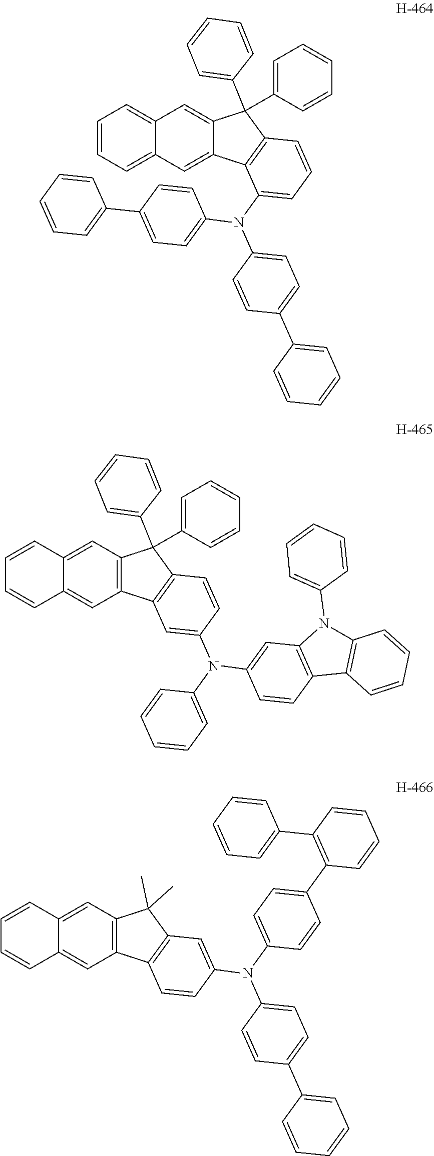 [SMILES: c1ccc(-c2ccc(N(c3ccc(-c4ccccc4)cc3)c3cccc4c3-c3cc5ccccc5cc3C4(c3ccccc3)c3ccccc3)cc2)cc1 ; c1ccc(N(c2ccc3c(c2)-c2cc4ccccc4cc2C3(c2ccccc2)c2ccccc2)c2ccc3c4ccccc4n(-c4ccccc4)c3c2)cc1 ; CC1(C)c2cc(N(c3ccc(-c4ccccc4)cc3)c3ccc(-c4ccccc4-c4ccccc4)cc3)ccc2-c2cc3ccccc3cc21]

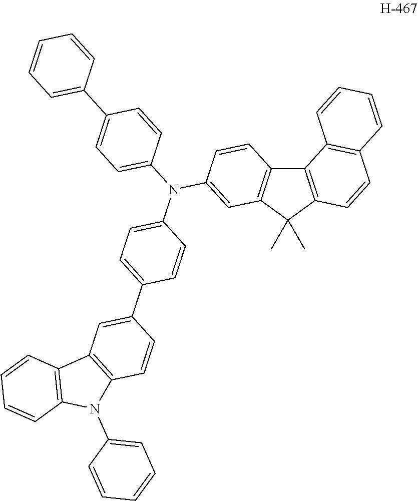 [SMILES: CC1(C)c2cc(N(c3ccc(-c4ccccc4)cc3)c3ccc(-c4ccc5c(c4)c4ccccc4n5-c4ccccc4)cc3)ccc2-c2c1ccc1ccccc21]

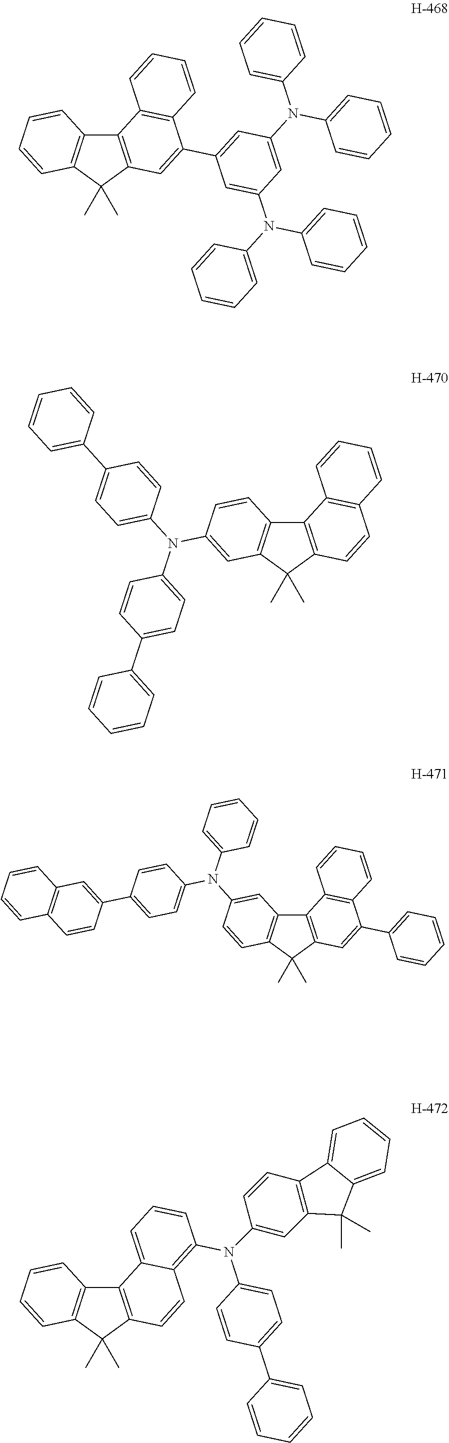 [SMILES: CC1(C)c2ccccc2-c2c1cc(-c1cc(N(c3ccccc3)c3ccccc3)cc(N(c3ccccc3)c3ccccc3)c1)c1ccccc21 ; CC1(C)c2cc(N(c3ccc(-c4ccccc4)cc3)c3ccc(-c4ccccc4)cc3)ccc2-c2c1ccc1ccccc21 ; CC1(C)c2ccc(N(c3ccccc3)c3ccc(-c4ccc5ccccc5c4)cc3)cc2-c2c1cc(-c1ccccc1)c1ccccc21 ; CC1(C)c2ccccc2-c2ccc(N(c3ccc(-c4ccccc4)cc3)c3cccc4c5c(ccc34)C(C)(C)c3ccccc3-5)cc21]

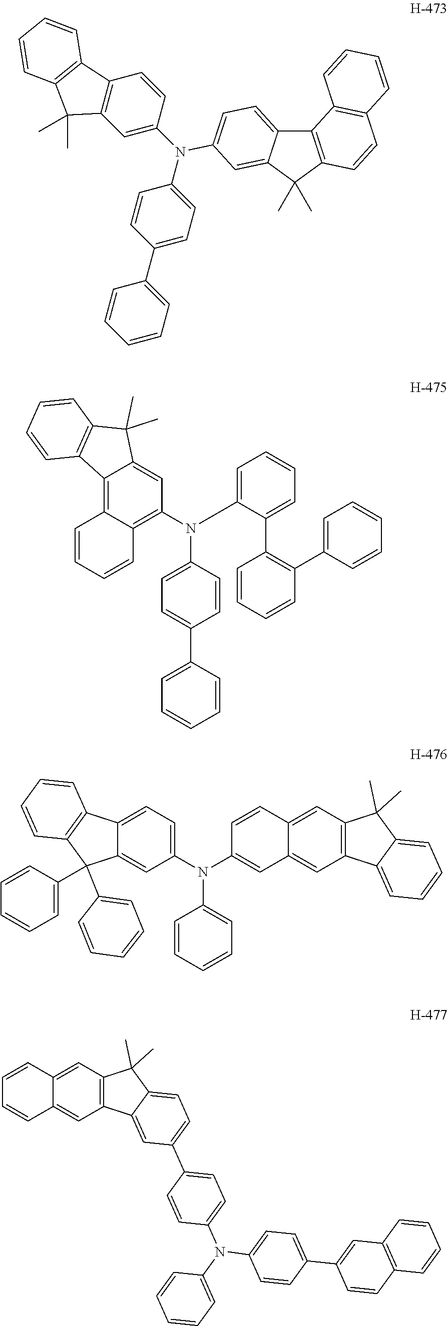 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2c1cc(N(c1ccc(-c3ccccc3)cc1)c1ccccc1-c1ccccc1-c1ccccc1)c1ccccc21 ; CC1(C)c2ccccc2-c2cc3cc(N(c4ccccc4)c4ccc5c(c4)C(c4ccccc4)(c4ccccc4)c4ccccc4-5)ccc3cc21 ; CC1(C)c2ccc(-c3ccc(N(c4ccccc4)c4ccc(-c5ccc6ccccc6c5)cc4)cc3)cc2-c2cc3ccccc3cc21]

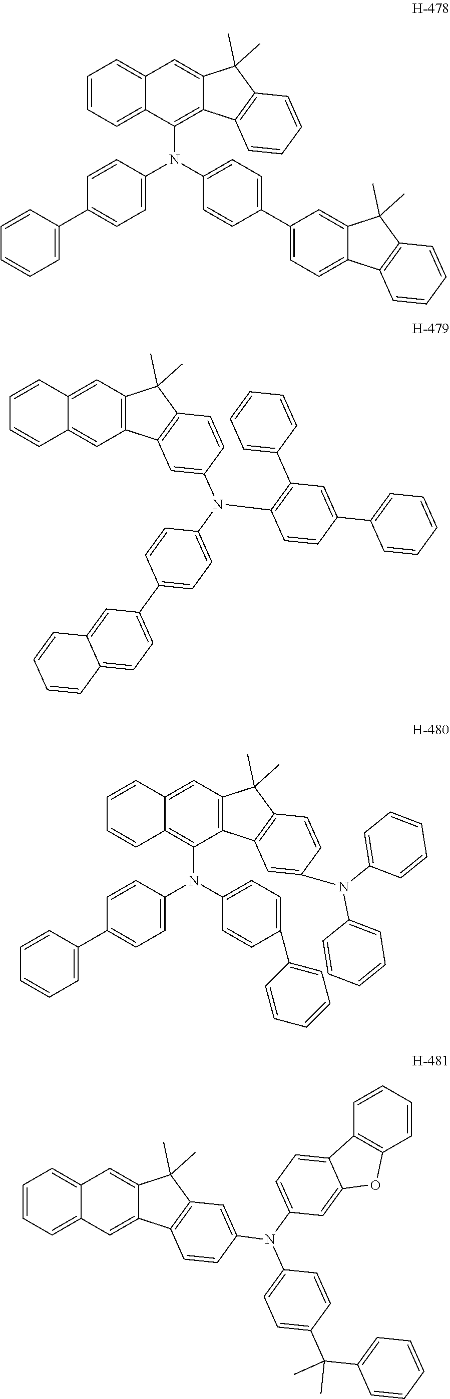 [SMILES: CC1(C)c2ccccc2-c2ccc(-c3ccc(N(c4ccc(-c5ccccc5)cc4)c4c5c(cc6ccccc46)C(C)(C)c4ccccc4-5)cc3)cc21 ; CC1(C)c2ccc(N(c3ccc(-c4ccc5ccccc5c4)cc3)c3ccc(-c4ccccc4)cc3-c3ccccc3)cc2-c2cc3ccccc3cc21 ; CC1(C)c2ccc(N(c3ccccc3)c3ccccc3)cc2-c2c1cc1ccccc1c2N(c1ccc(-c2ccccc2)cc1)c1ccc(-c2ccccc2)cc1 ; CC(C)(c1ccccc1)c1ccc(N(c2ccc3c(c2)C(C)(C)c2cc4ccccc4cc2-3)c2ccc3c(c2)oc2ccccc23)cc1]

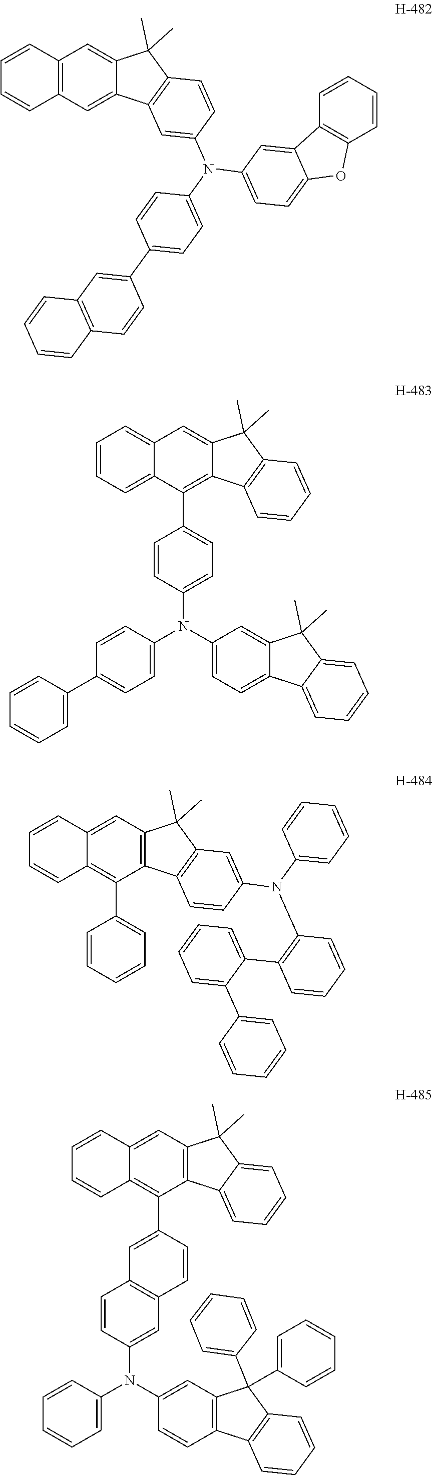

The host compound represented by Formula 1 according to the present disclosure may be prepared as shown in the following Reaction Scheme 1, but is not limited thereto, and may also be prepared by synthetic methods known to those skilled in the art.

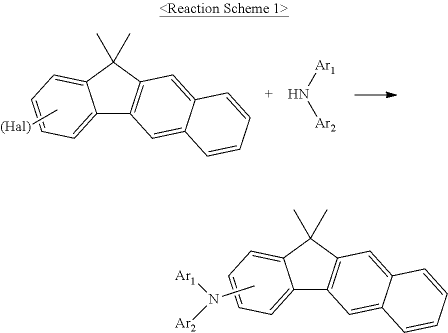

In Reaction Scheme 1, the definition of each of the substituents is as defined in Formula 1.

As described above, exemplary synthesis examples of the compounds represented by Formula 1 according to the present disclosure are described, but they are based on a Buchwald-Hartwig cross coupling reaction, N-arylation reaction, H-mont-mediated etherification reaction, Miyaura borylation reaction, Suzuki cross-coupling reaction, intramolecular acid-induced cyclization reaction, Pd(II)-catalyzed oxidative cyclization reaction, Grignard reaction, Heck reaction, cyclic dehydration reaction, SN₁ substitution reaction, SN₂ substitution reaction, and phosphine-mediated reductive cyclization reaction, etc. It will be understood by one skilled in the art that the above reaction proceeds even if other substituents defined in Formula 1 other than the substituents described in the specific synthesis examples are bonded.

The second host compound as another host material according to one embodiment may be represented by the following Formula 2.

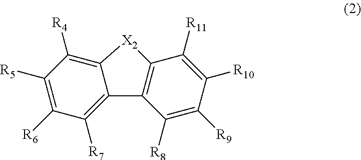

In Formula 2,
X₂ represents —O—, —S— or —Se—;
R₄ to R₆ and R₈ to R₁₁ each independently represent, hydrogen, deuterium, or a substituted or unsubstituted (C6-C30)aryl; provided that the (C6-C30)aryl does not include a substituted or unsubstituted fluorenyl; and
R₇ represents the following Formula 2-a;

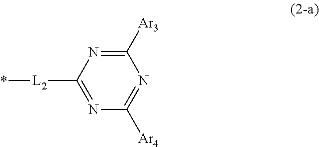

In Formula 2-a,
L₂ represents a single bond, a substituted or unsubstituted (C6-C30)arylene, or a substituted or unsubstituted (3-to 30-membered)heteroarylene; and
Ar₃ and Ar₄ each independently represent, a substituted or unsubstituted (C6-C30)aryl, but do not include a substituted or unsubstituted fluorenyl.

In one embodiment, R₄ to R₆ and R₈ to R₁₁ each independently may be, hydrogen, deuterium, or (C6-C25)aryl unsubstituted or substituted with deuterium or (C6-C30)aryl, preferably hydrogen, deuterium, or (C6-C25)aryl unsubstituted or substituted with deuterium or (C6-C25)aryl, more preferably hydrogen, deuterium, or (C6-C18)aryl unsubstituted or substituted with deuterium or (C6-C18)aryl.

In one embodiment, when R₄ to R₆ and R₈ to R₁₁ are a substituted or unsubstituted (C6-C30)aryl, they do not include a substituted or unsubstituted fluorenyl. For example, R₄ to R₆ and R₈ to R₁₁ each independently may be hydrogen, deuterium, phenyl unsubstituted or substituted with naphthyl, naphthyl unsubstituted or substituted with phenyl, a substituted or unsubstituted p-biphenyl, a substituted or unsubstituted o-biphenyl, or a substituted or unsubstituted triphenylenyl.

In one embodiment, L₂ may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₂ may be a single bond, a substituted or unsubstituted phenylene, or naphthylene unsubstituted or substituted with deuterium.

In one embodiment, Ar3 and Ar4 each independently may be a substituted or unsubstituted (C6-C30) aryl, except for a substituted or unsubstituted fluorenyl. Preferably Ar₃ and Ar₄ each independently may be (C6-C25)aryl unsubstituted or substituted with at least one of deuterium; (C6-C30)aryl; (5-to 30-membered)heteroaryl; and tri(C6-C30)arylsilyl, more preferably (C6-C18)aryl unsubstituted or substituted with at least one of deuterium; (C6-C25)aryl; (5-to 25-membered)heteroaryl; and tri(C6-C25)arylsilyl. For example, Ar₃ and Ar₄ each independently may be phenyl unsubstituted or substituted with at least one of deuterium; naphthyl; dibenzofuranyl; carbazolyl; and triphenylsilyl, naphthyl unsubstituted or substituted with at least one of deuterium; phenyl; and biphenyl, p-biphenyl unsubstituted or substituted with at least one of deuterium; and phenyl, a substituted or unsubstituted p-terphenyl, a substituted or unsubstituted m-terphenyl, a substituted or unsubstituted phenanthrenyl, a substituted or unsubstituted chrysenyl, or a substituted or unsubstituted triphenylenyl. For example, Ar₃ and Ar₄ each independently may be selected from phenyl unsubstituted or substituted with deuterium, naphthyl unsubstituted or substituted with deuterium, biphenyl unsubstituted or substituted with deuterium, terphenyl unsubstituted or substituted with deuterium, phenanthrenyl unsubstituted or substituted with deuterium, chrysenyl unsubstituted or substituted with deuterium, or a combination thereof. For example, at least one of Ars and Ara may be naphthyl unsubstituted or substituted with deuterium or phenyl, a substituted or unsubstituted phenanthrenyl, or a substituted or unsubstituted chrysenyl.

According to one embodiment, the second host compound represented by Formula 2 may be more specifically illustrated by the following compounds, but is not limited thereto.

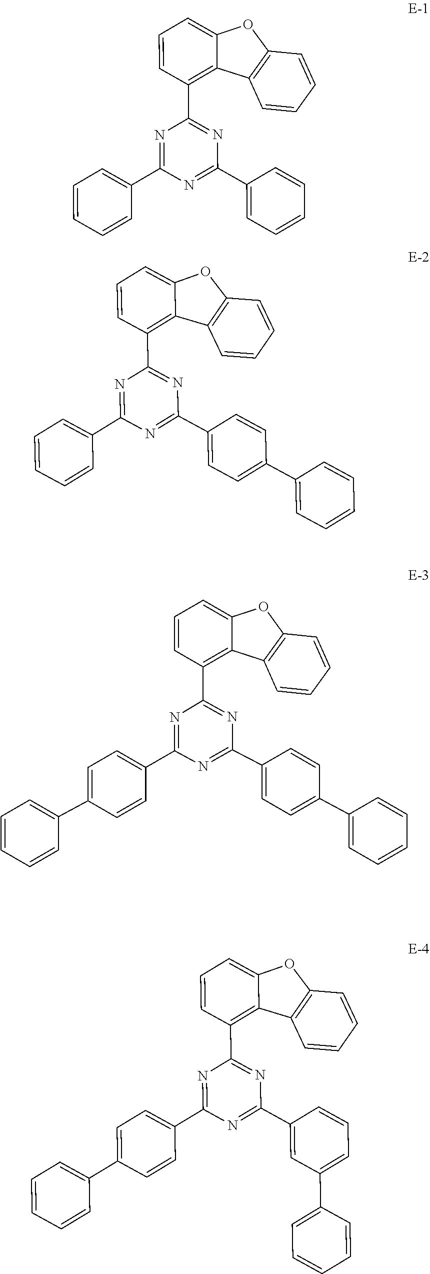 [SMILES: c1ccc(-c2nc(-c3ccccc3)nc(-c3cccc4oc5ccccc5c34)n2)cc1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1]

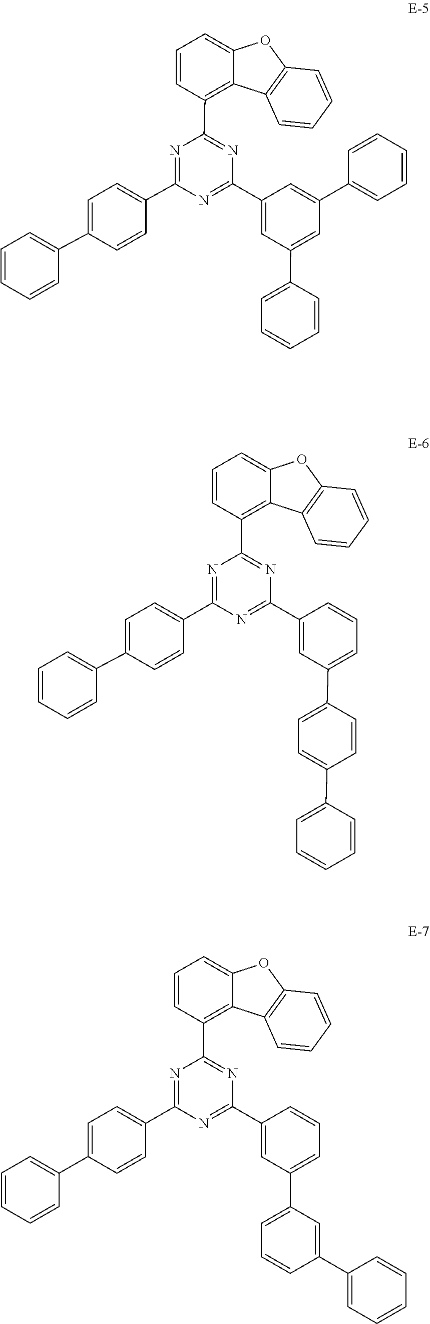 [SMILES: c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cccc(-c4nc(-c5ccc(-c6ccccc6)cc5)nc(-c5cccc6oc7ccccc7c56)n4)c3)cc2)cc1 ; c1ccc(-c2ccc(-c3nc(-c4cccc(-c5cccc(-c6ccccc6)c5)c4)nc(-c4cccc5oc6ccccc6c45)n3)cc2)cc1]

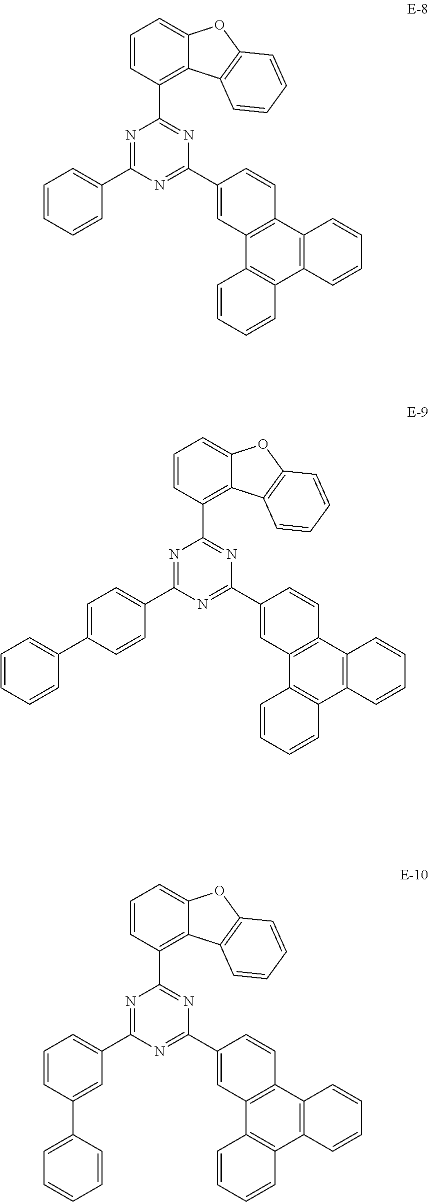 [SMILES: c1ccc(-c2nc(-c3ccc4c5ccccc5c5ccccc5c4c3)nc(-c3cccc4oc5ccccc5c34)n2)cc1 ; c1ccc(-c2ccc(-c3nc(-c4ccc5c6ccccc6c6ccccc6c5c4)nc(-c4cccc5oc6ccccc6c45)n3)cc2)cc1 ; c1ccc(-c2cccc(-c3nc(-c4ccc5c6ccccc6c6ccccc6c5c4)nc(-c4cccc5oc6ccccc6c45)n3)c2)cc1]

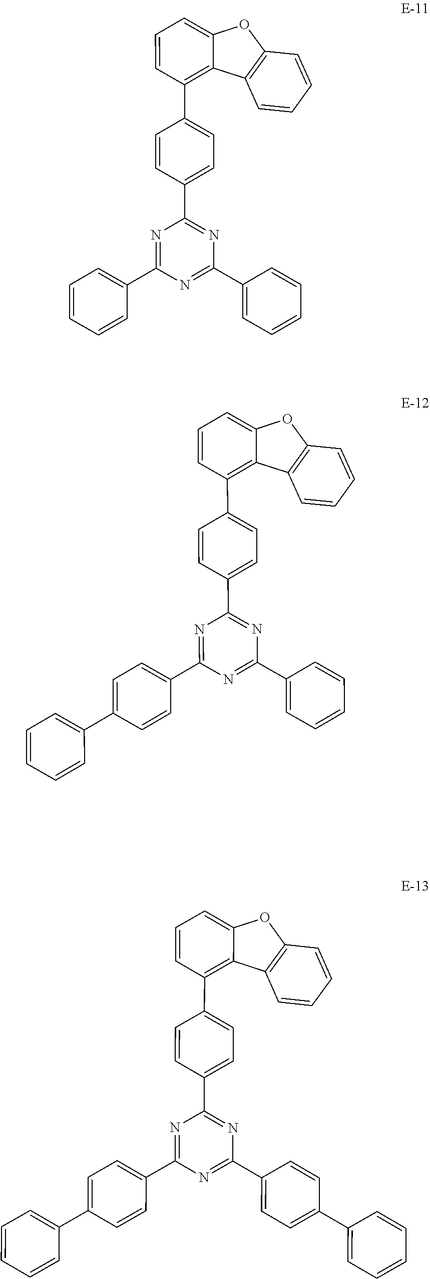 [SMILES: c1ccc(-c2nc(-c3ccccc3)nc(-c3ccc(-c4cccc5oc6ccccc6c45)cc3)n2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1]

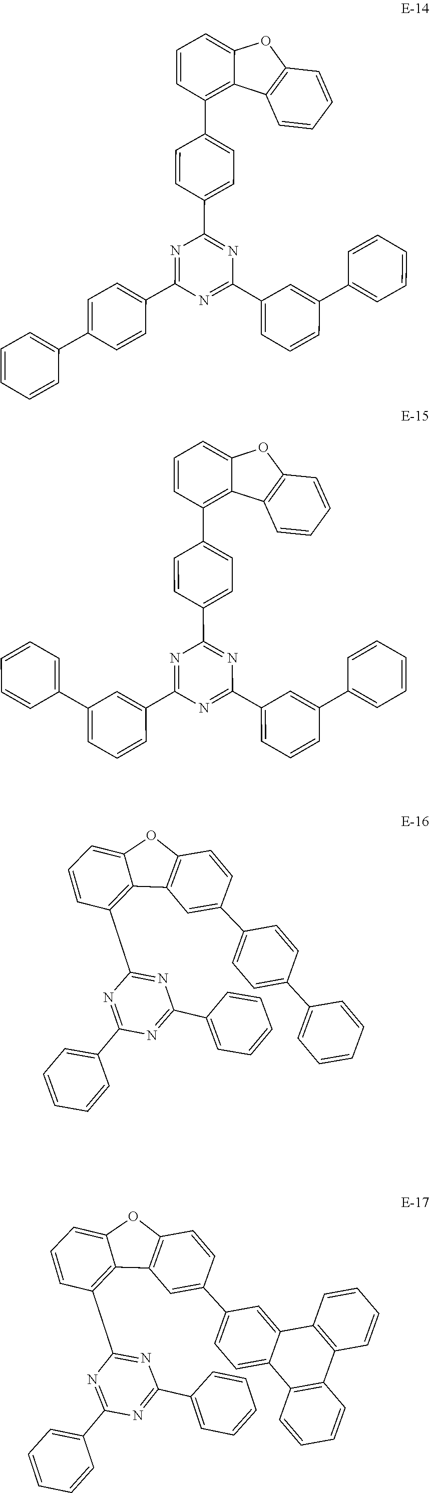 [SMILES: c1ccc(-c2ccc(-c3nc(-c4ccc(-c5cccc6oc7ccccc7c56)cc4)nc(-c4cccc(-c5ccccc5)c4)n3)cc2)cc1 ; c1ccc(-c2cccc(-c3nc(-c4ccc(-c5cccc6oc7ccccc7c56)cc4)nc(-c4cccc(-c5ccccc5)c4)n3)c2)cc1 ; c1ccc(-c2ccc(-c3ccc4oc5cccc(-c6nc(-c7ccccc7)nc(-c7ccccc7)n6)c5c4c3)cc2)cc1 ; c1ccc(-c2nc(-c3ccccc3)nc(-c3cccc4oc5ccc(-c6ccc7c8ccccc8c8ccccc8c7c6)cc5c34)n2)cc1]

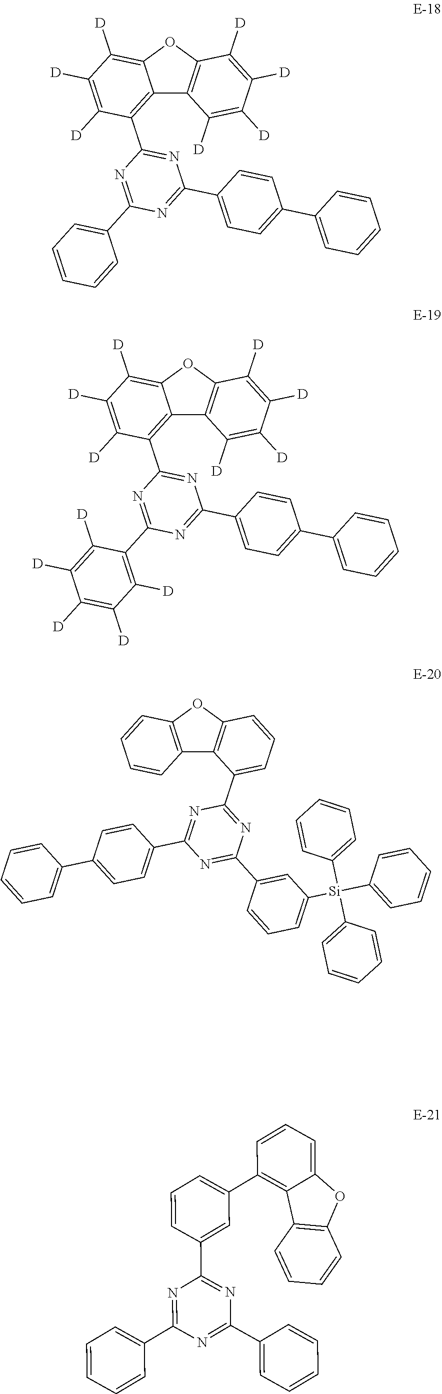 [SMILES: [2H]c1c([2H])c([2H])c2c(oc3c([2H])c([2H])c([2H])c(-c4nc(-c5ccccc5)nc(-c5ccc(-c6ccccc6)cc5)n4)c32)c1[2H] ; [2H]c1c([2H])c([2H])c(-c2nc(-c3ccc(-c4ccccc4)cc3)nc(-c3c([2H])c([2H])c([2H])c4oc5c([2H])c([2H])c([2H])c([2H])c5c34)n2)c([2H])c1[2H] ; c1ccc(-c2ccc(-c3nc(-c4cccc([Si](c5ccccc5)(c5ccccc5)c5ccccc5)c4)nc(-c4cccc5oc6ccccc6c45)n3)cc2)cc1 ; c1ccc(-c2nc(-c3ccccc3)nc(-c3cccc(-c4cccc5oc6ccccc6c45)c3)n2)cc1]

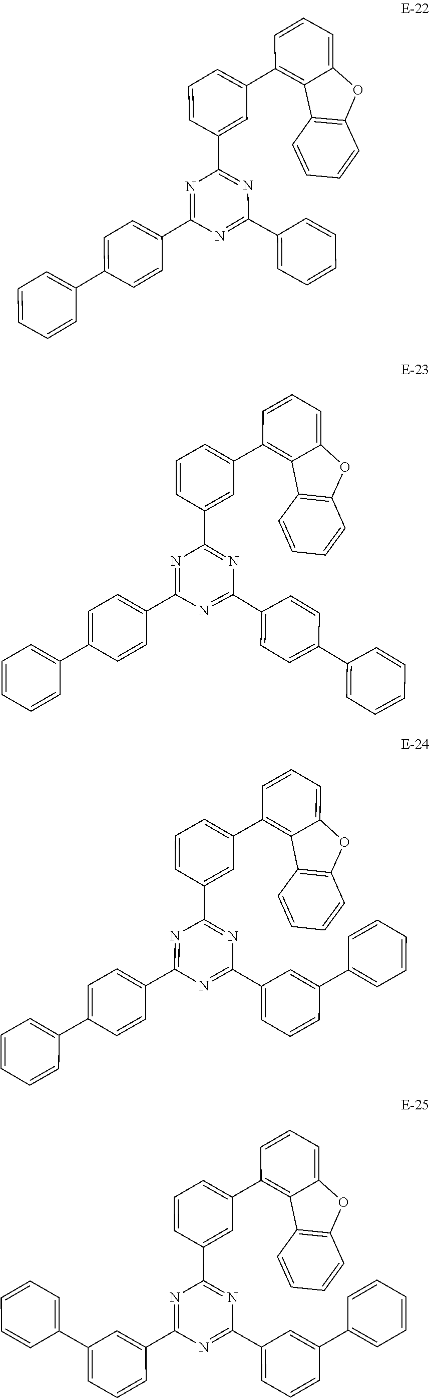 [SMILES: c1ccc(-c2ccc(-c3nc(-c4ccccc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4cccc(-c5ccccc5)c4)nc(-c4cccc(-c5cccc6oc7ccccc7c56)c4)n3)cc2)cc1 ; c1ccc(-c2cccc(-c3nc(-c4cccc(-c5ccccc5)c4)nc(-c4cccc(-c5cccc6oc7ccccc7c56)c4)n3)c2)cc1]

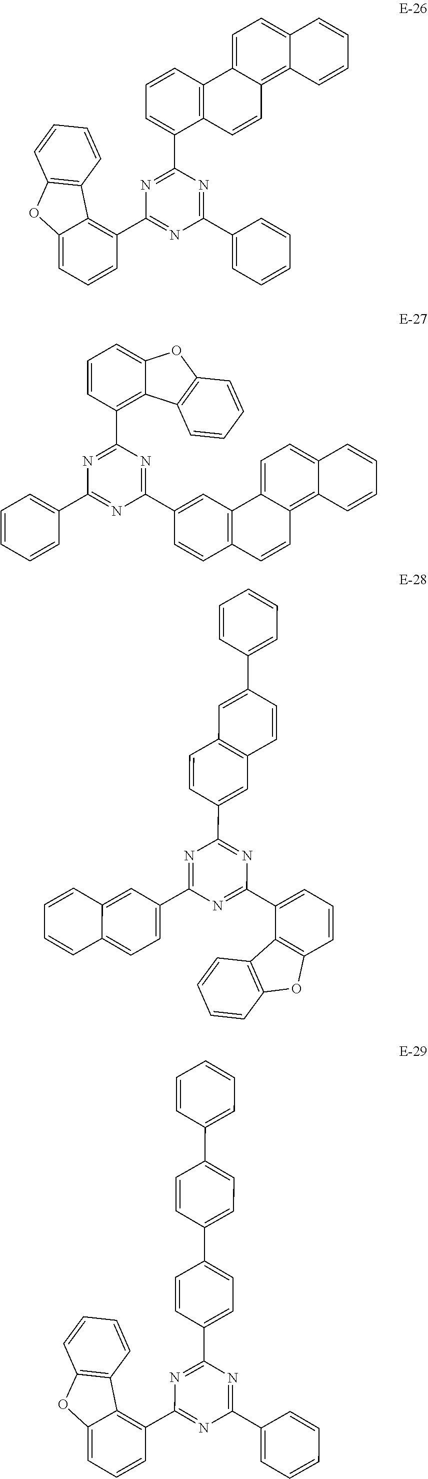

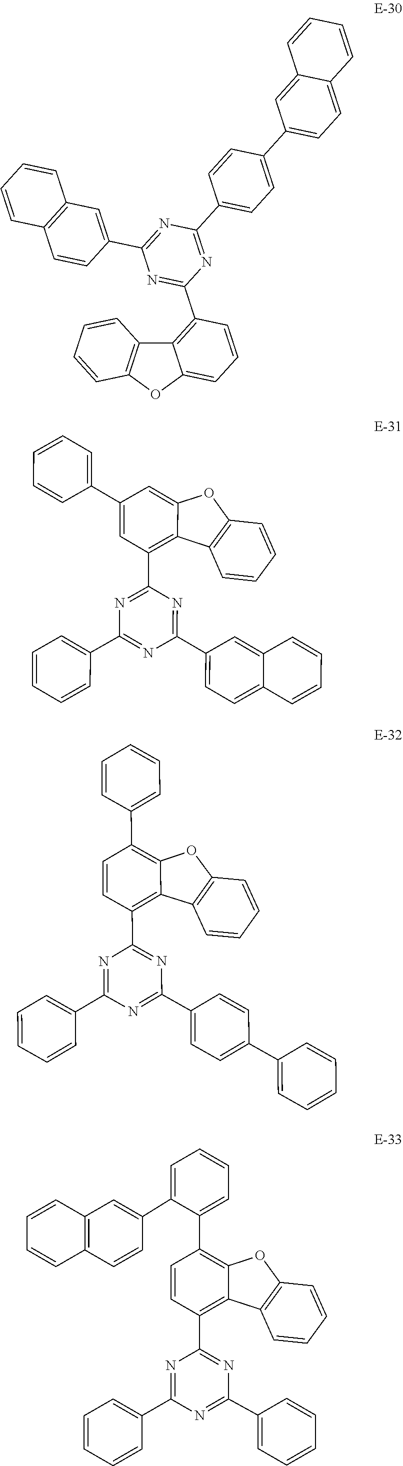

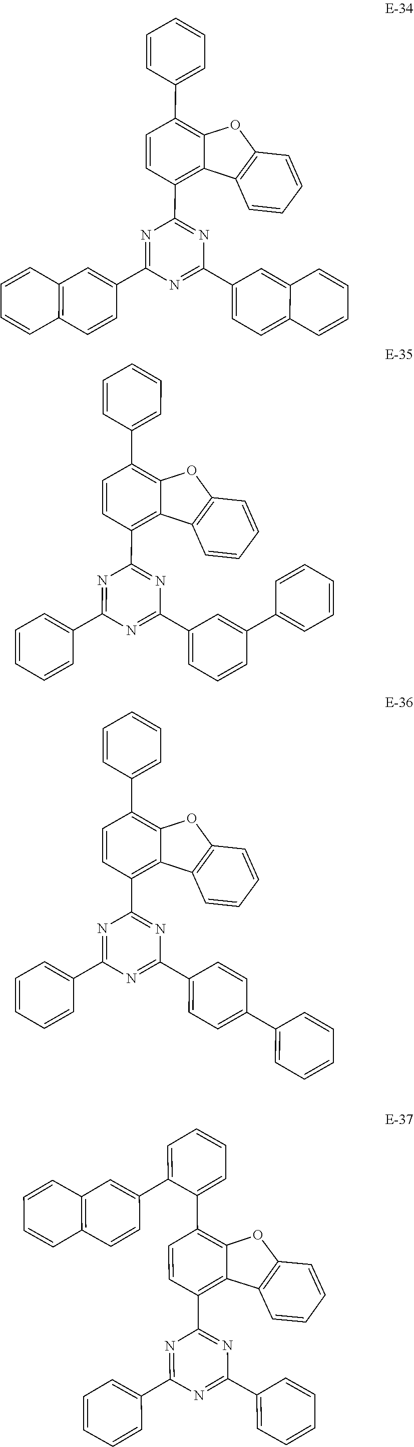

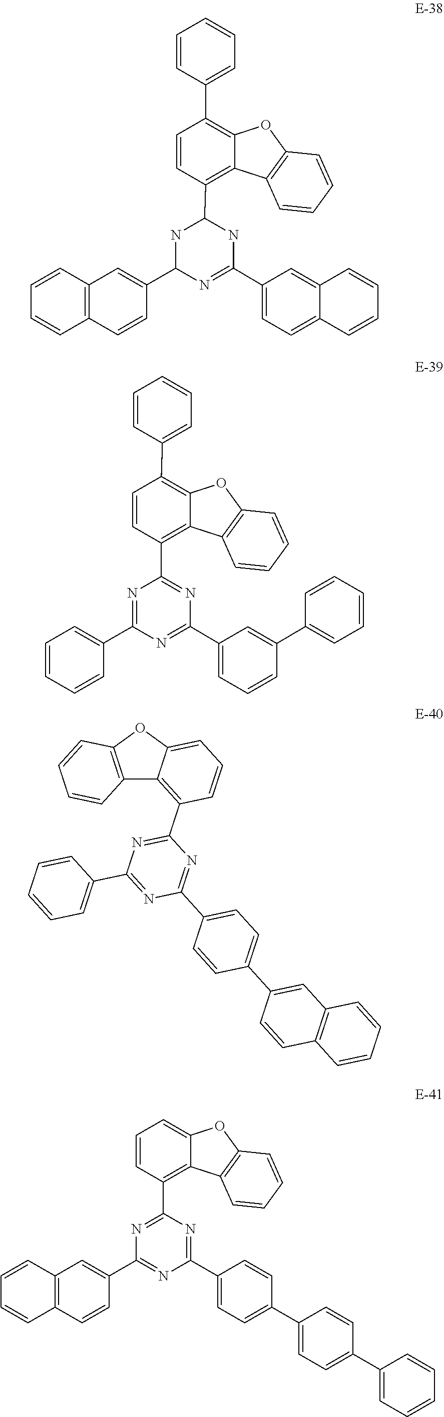

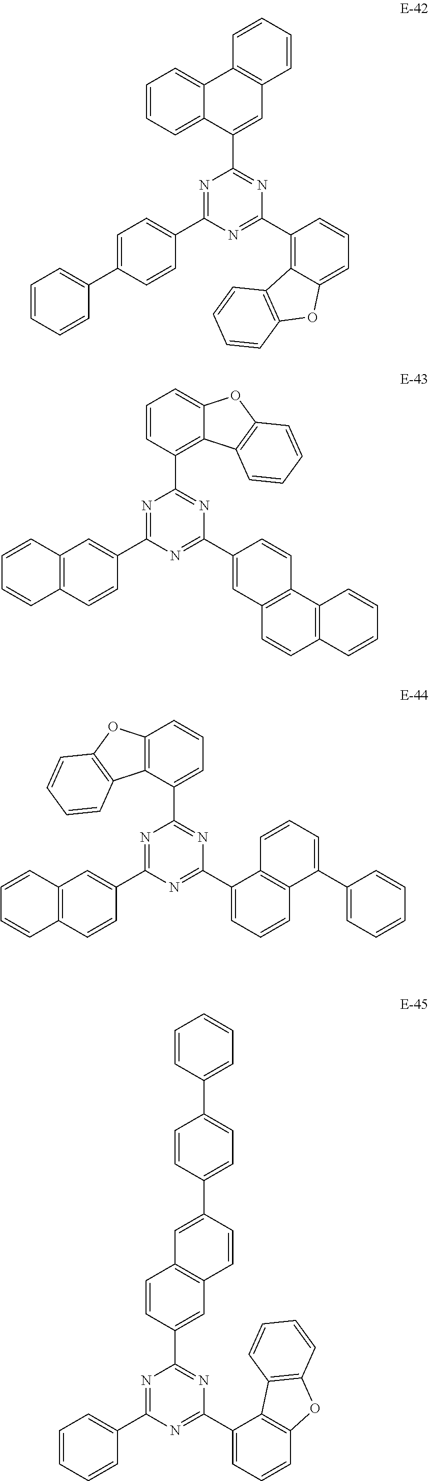 [SMILES: c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4ccc5c(ccc6ccccc65)c4)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1]

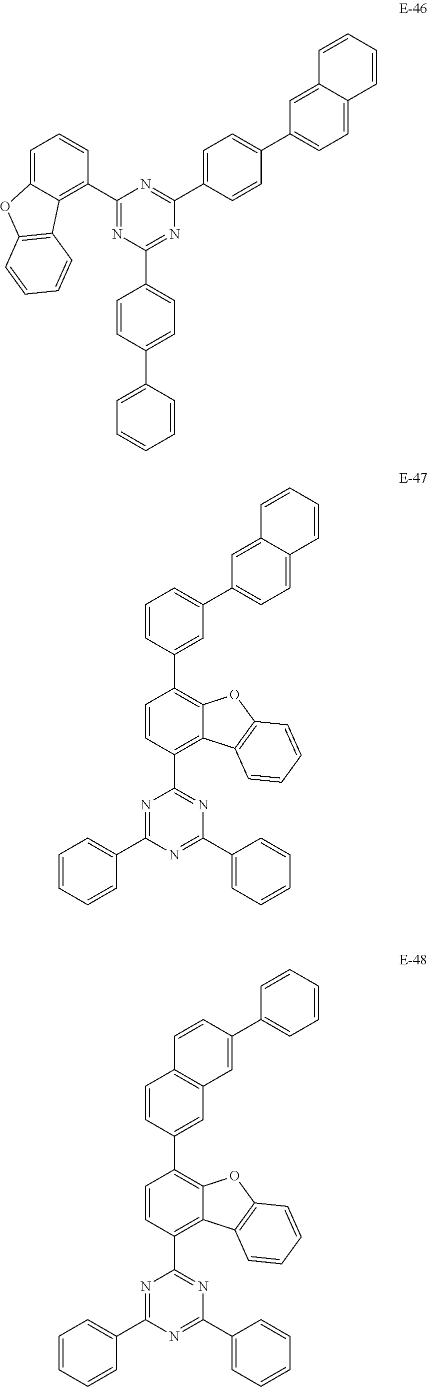 [SMILES: c1ccc(-c2ccc(-c3nc(-c4ccc(-c5ccc6ccccc6c5)cc4)nc(-c4cccc5oc6ccccc6c45)n3)cc2)cc1 ; c1ccc(-c2nc(-c3ccccc3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccccc6)n5)c5c4oc4ccccc45)cc3c2)cc1]

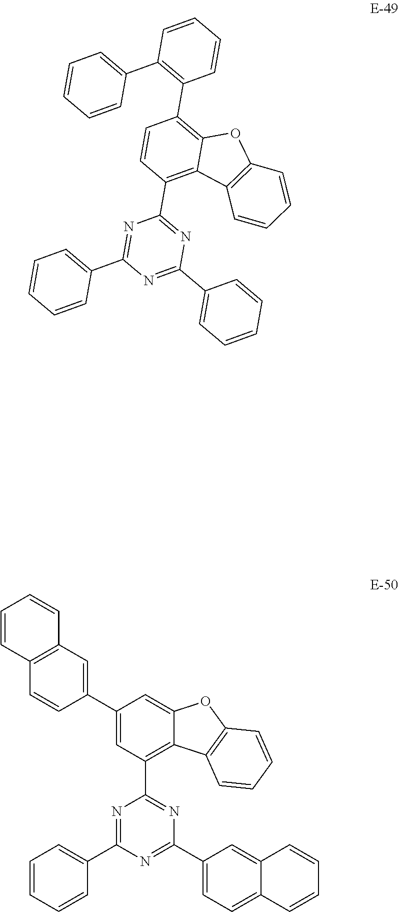 [SMILES: c1ccc(-c2nc(-c3ccccc3)nc(-c3ccc(-c4ccccc4-c4ccccc4)c4oc5ccccc5c34)n2)cc1 ; c1ccc(-c2nc(-c3ccc4ccccc4c3)nc(-c3cc(-c4ccc5ccccc5c4)cc4oc5ccccc5c34)n2)cc1]

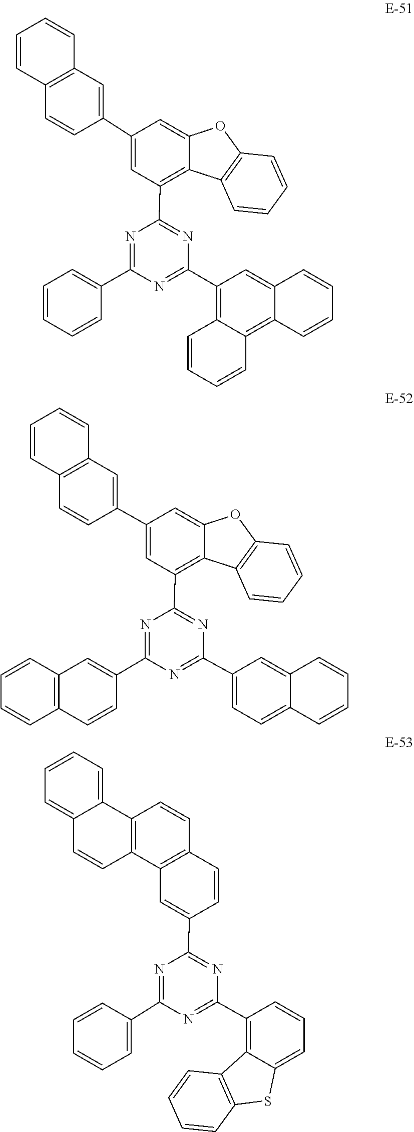

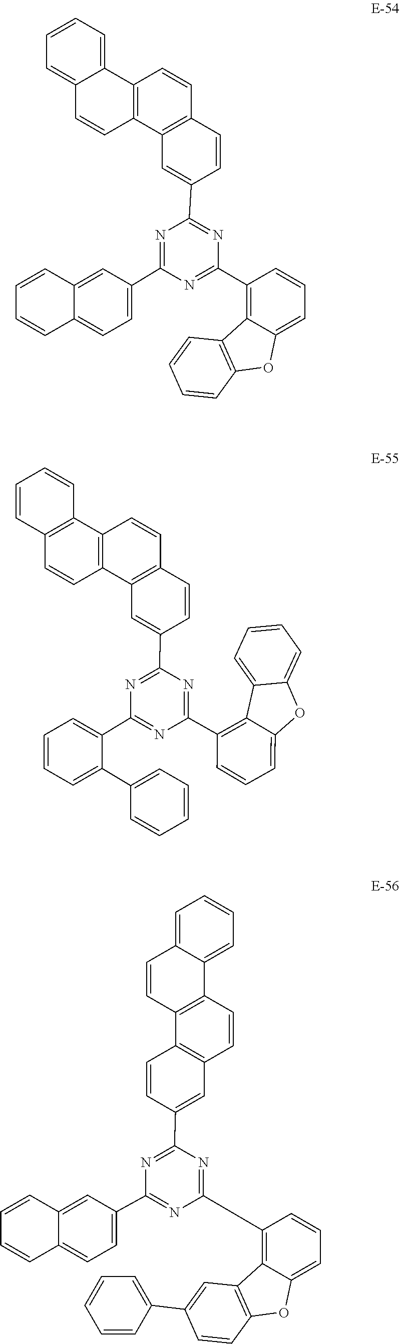 [SMILES: c1ccc2cc(-c3nc(-c4ccc5ccc6c7ccccc7ccc6c5c4)nc(-c4cccc5oc6ccccc6c45)n3)ccc2c1 ; c1ccc(-c2ccccc2-c2nc(-c3ccc4ccc5c6ccccc6ccc5c4c3)nc(-c3cccc4oc5ccccc5c34)n2)cc1 ; c1ccc(-c2ccc3oc4cccc(-c5nc(-c6ccc7ccccc7c6)nc(-c6ccc7c(ccc8c9ccccc9ccc78)c6)n5)c4c3c2)cc1]

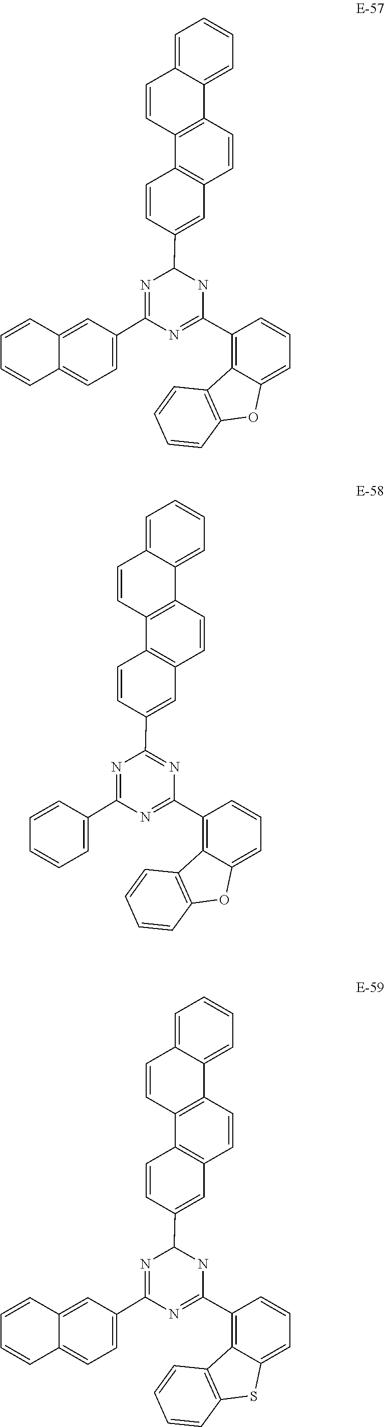

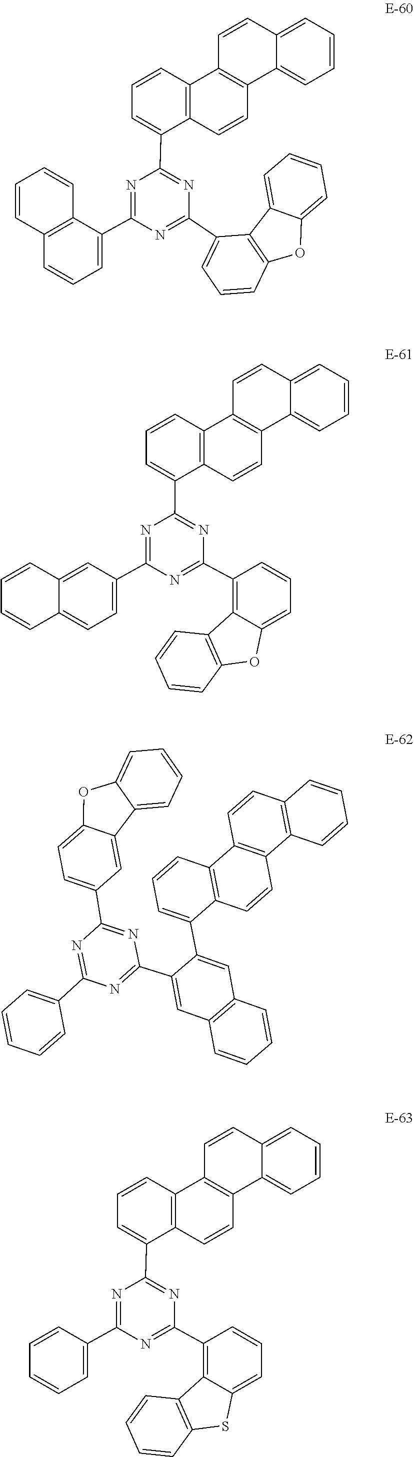 [SMILES: c1ccc2c(-c3nc(-c4cccc5c4ccc4c6ccccc6ccc54)nc(-c4cccc5oc6ccccc6c45)n3)cccc2c1 ; c1ccc2cc(-c3nc(-c4cccc5c4ccc4c6ccccc6ccc54)nc(-c4cccc5oc6ccccc6c45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4sc5ccccc5c34)n2)cc1]

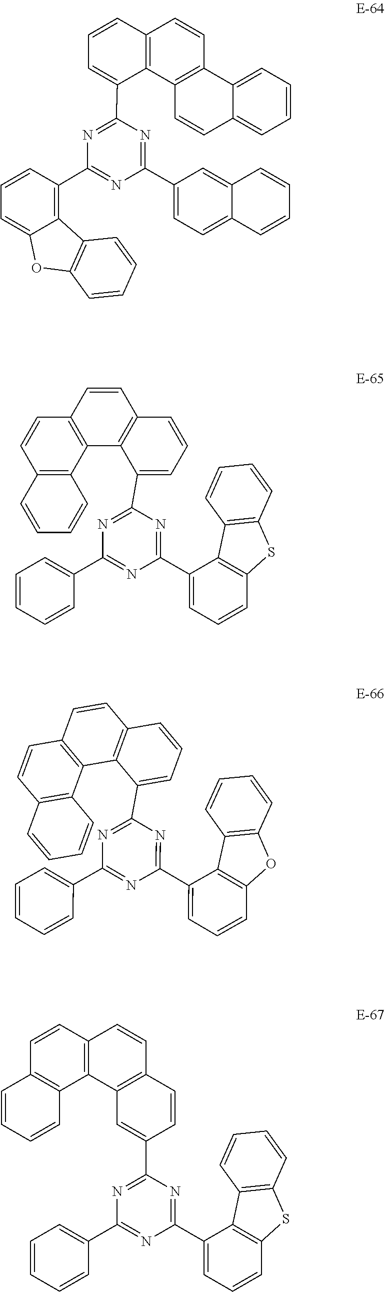 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3ccc4ccc5ccc6ccccc6c5c4c3)nc(-c3cccc4sc5ccccc5c34)n2)cc1]

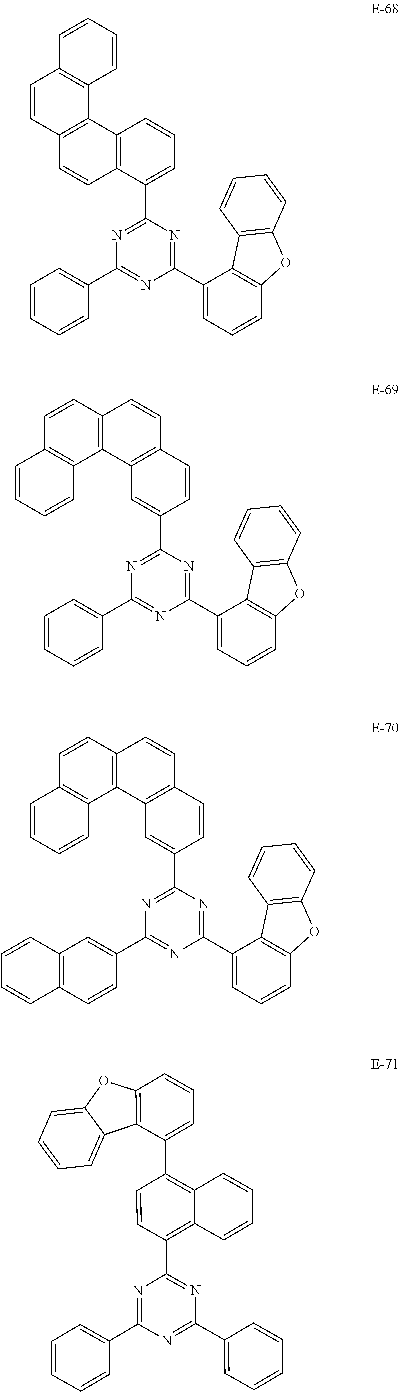 [SMILES: c1ccc(-c2nc(-c3cccc4c3ccc3ccc5ccccc5c34)nc(-c3cccc4oc5ccccc5c34)n2)cc1 ; c1ccc(-c2nc(-c3ccc4ccc5ccc6ccccc6c5c4c3)nc(-c3cccc4oc5ccccc5c34)n2)cc1 ; c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccccc3)nc(-c3ccc(-c4cccc5oc6ccccc6c45)c4ccccc34)n2)cc1]

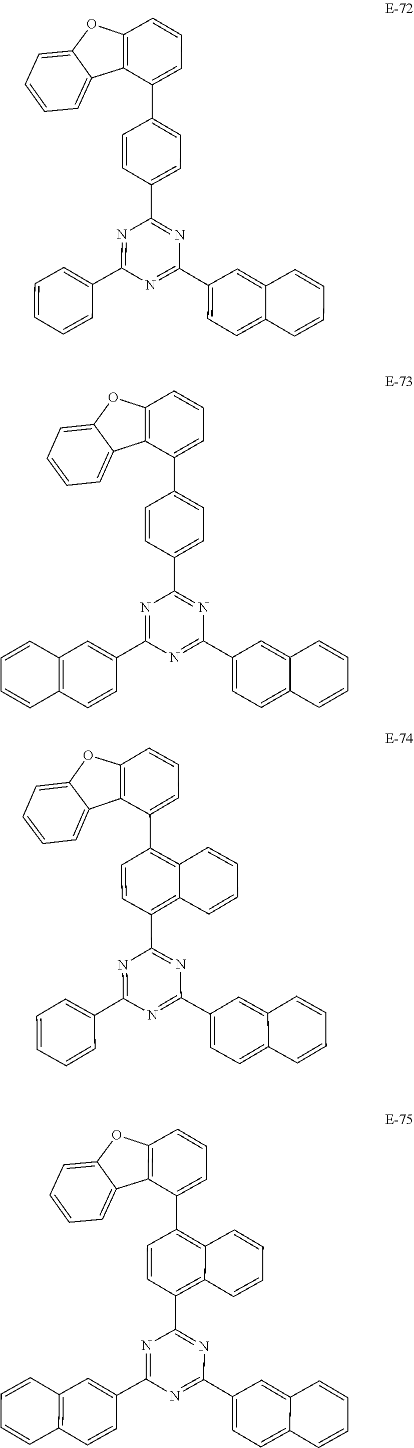

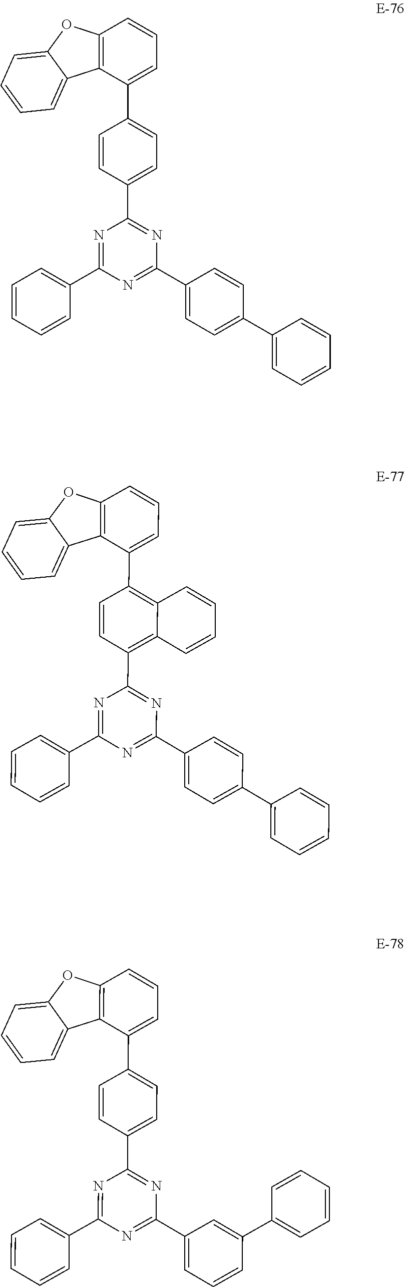 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1]

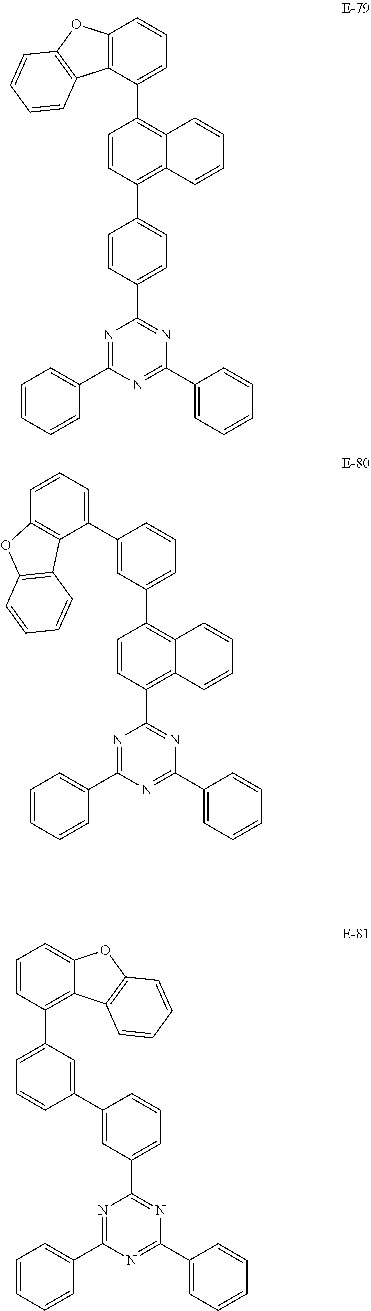

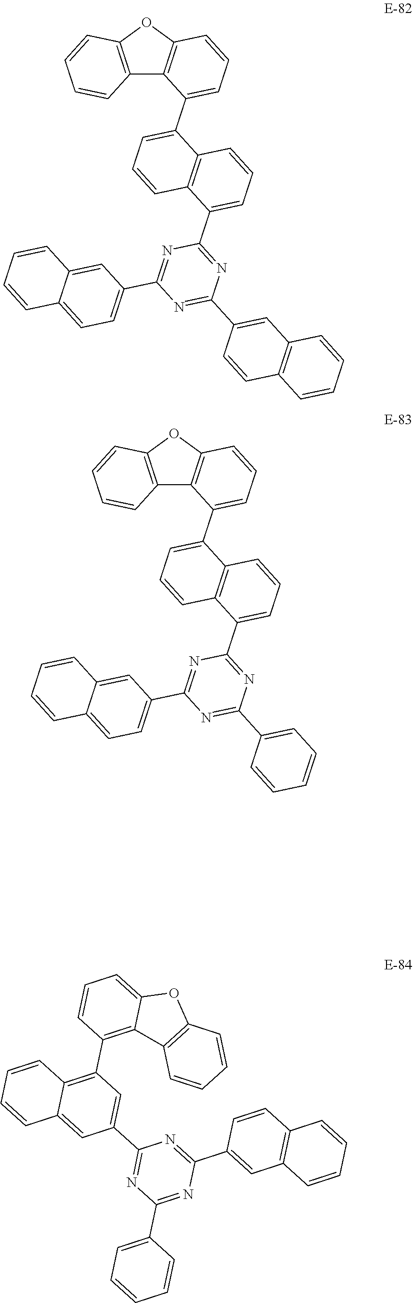 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1]

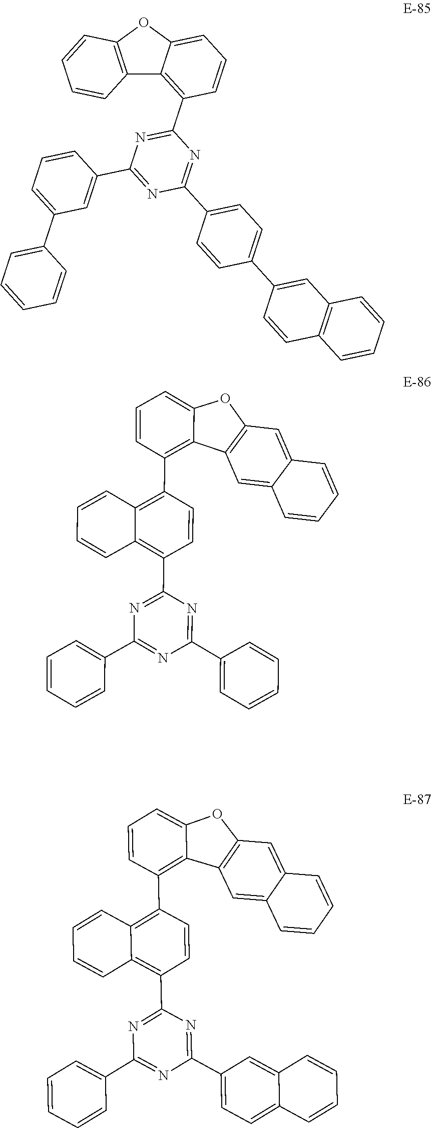 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccccc3)nc(-c3ccc(-c4cccc5oc6cc7ccccc7cc6c45)c4ccccc34)n2)cc1 ; c1ccc(-c2nc(-c3ccc4ccccc4c3)nc(-c3ccc(-c4cccc5oc6cc7ccccc7cc6c45)c4ccccc34)n2)cc1]

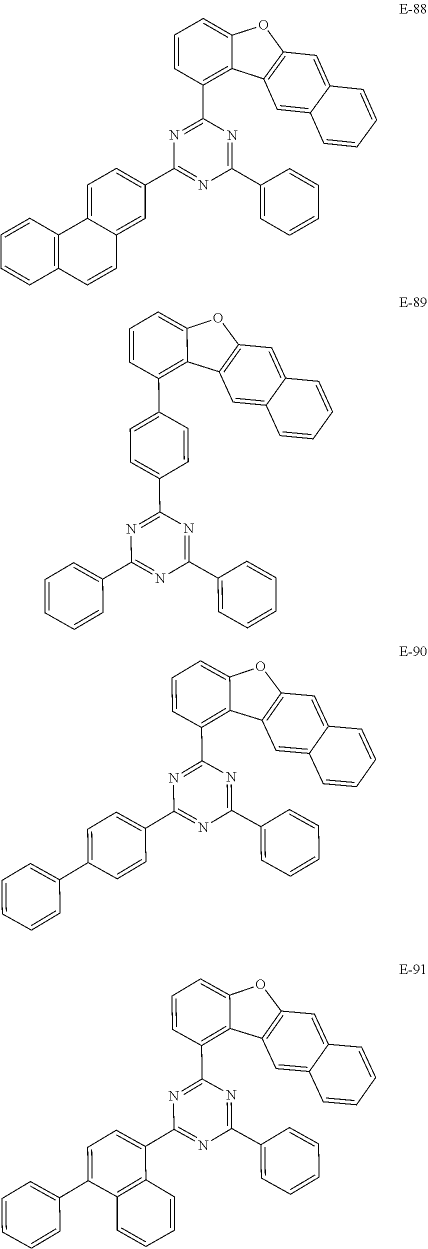

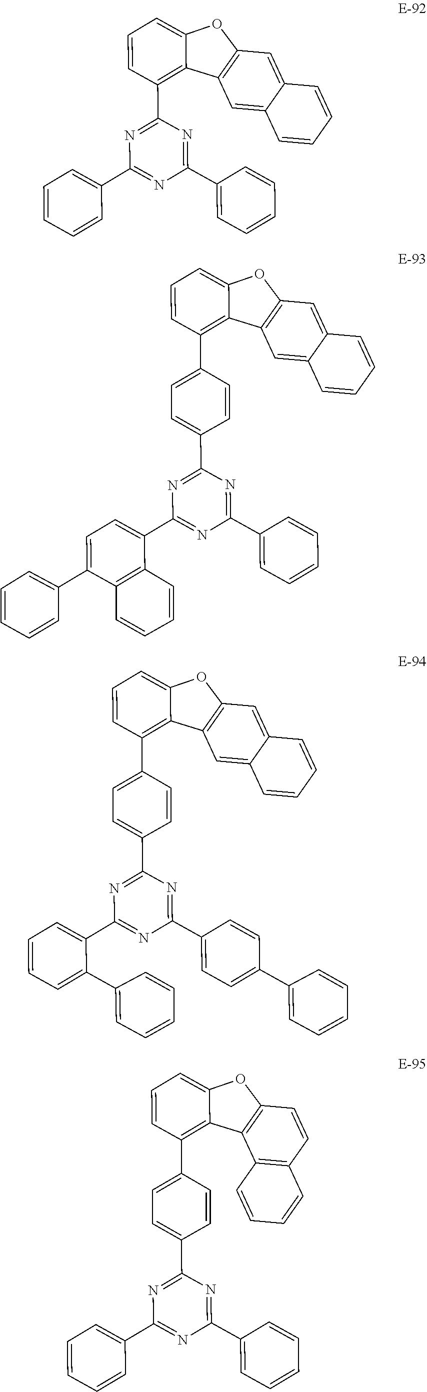

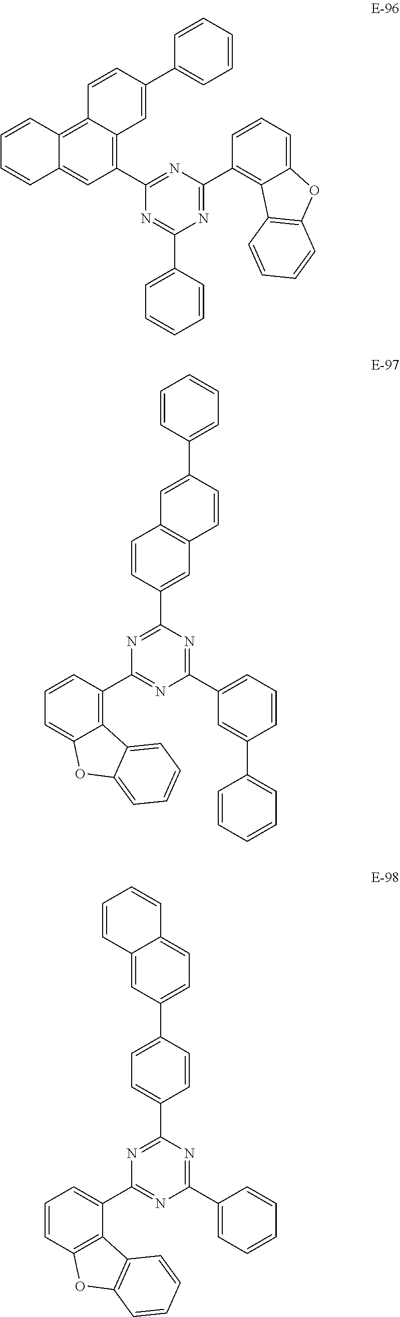

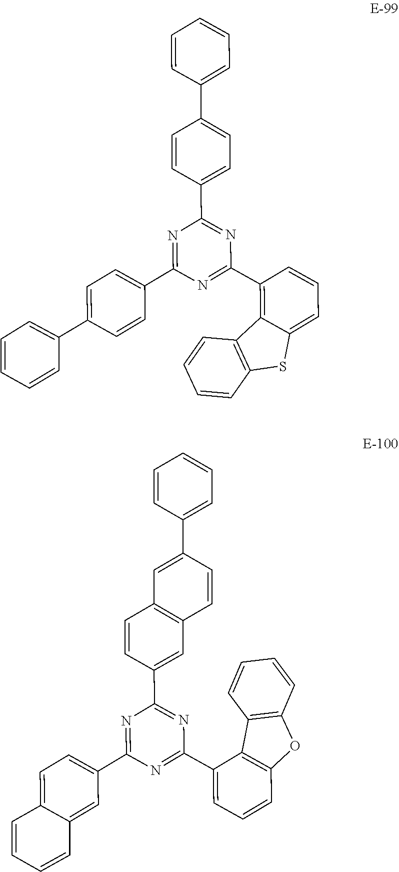 [SMILES: c1ccc(-c2ccc(-c3nc(-c4ccc(-c5ccccc5)cc4)nc(-c4cccc5sc6ccccc6c45)n3)cc2)cc1 ; c1ccc(-c2ccc3cc(-c4nc(-c5ccc6ccccc6c5)nc(-c5cccc6oc7ccccc7c56)n4)ccc3c2)cc1]

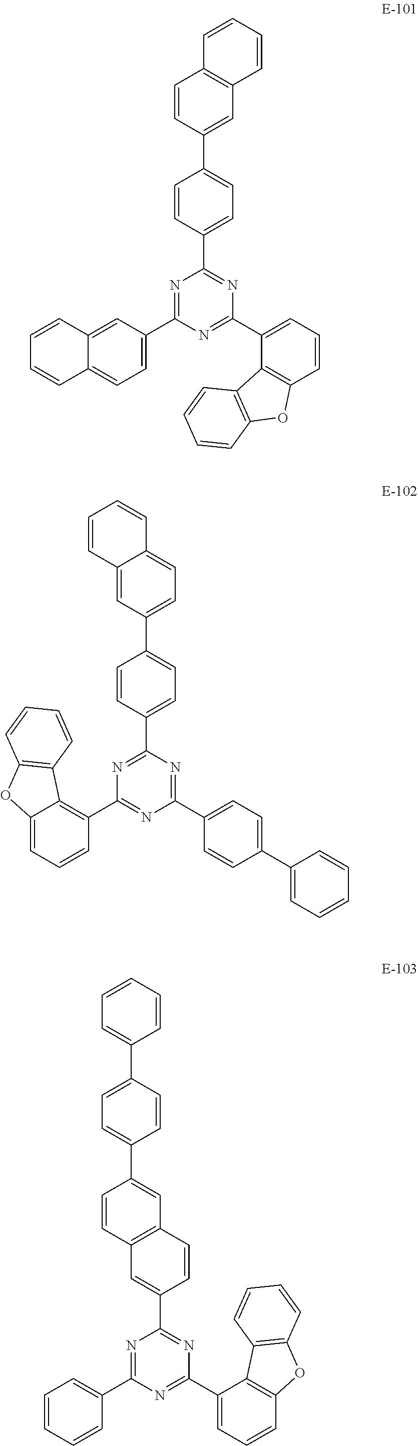 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4cccc5oc6ccccc6c45)n3)cc2)cc1 ; c1ccc(-c2ccc(-c3ccc4cc(-c5nc(-c6ccccc6)nc(-c6cccc7oc8ccccc8c67)n5)ccc4c3)cc2)cc1]

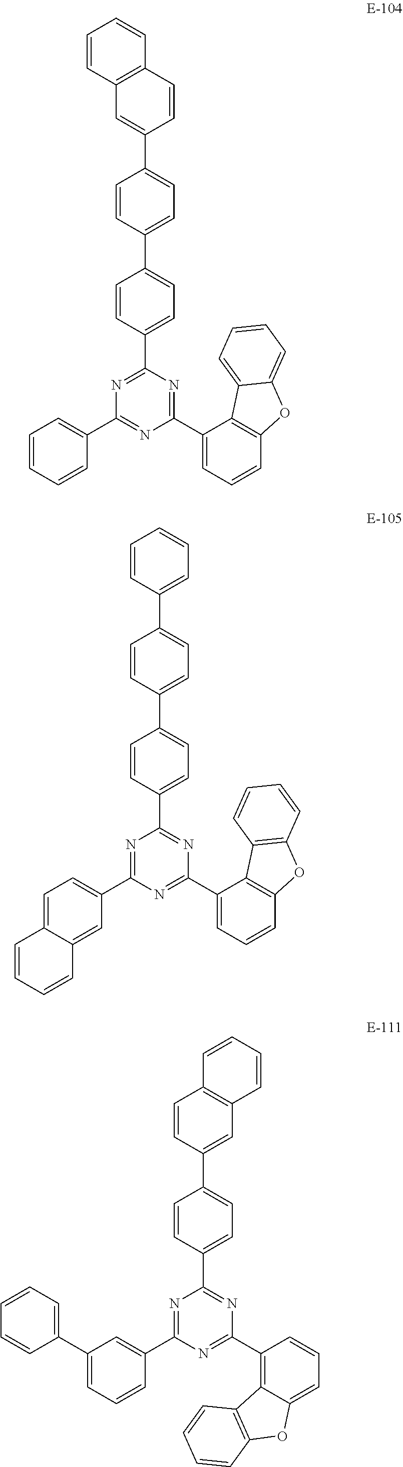 [SMILES: c1ccc(-c2nc(-c3ccc(-c4ccc(-c5ccc6ccccc6c5)cc4)cc3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2cccc(-c3nc(-c4ccc(-c5ccc6ccccc6c5)cc4)nc(-c4cccc5oc6ccccc6c45)n3)c2)cc1]

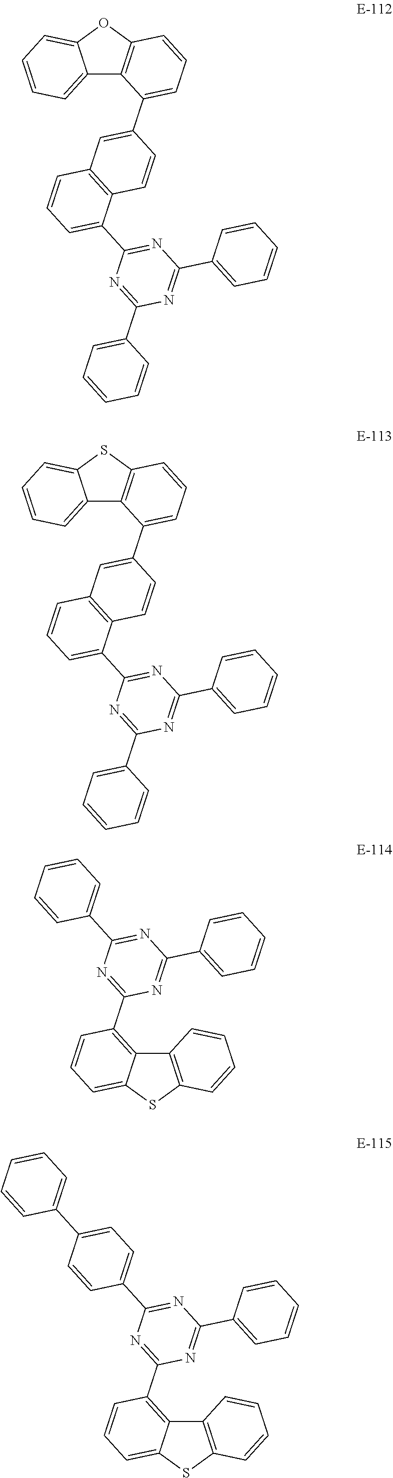

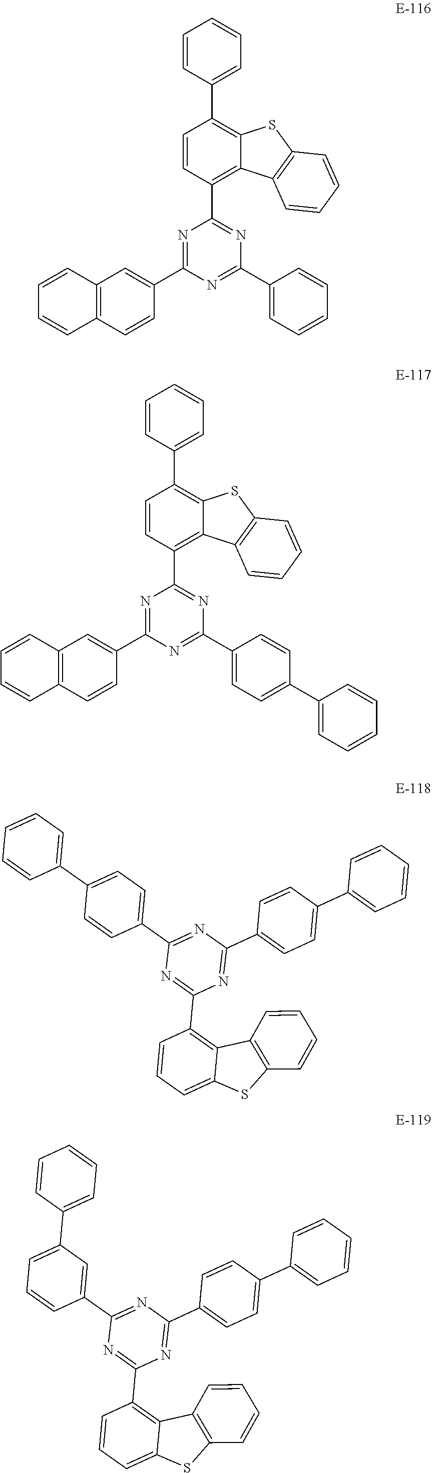 [SMILES: c1ccc(-c2nc(-c3ccc4ccccc4c3)nc(-c3ccc(-c4ccccc4)c4sc5ccccc5c34)n2)cc1 ; c1ccc(-c2ccc(-c3nc(-c4ccc5ccccc5c4)nc(-c4ccc(-c5ccccc5)c5sc6ccccc6c45)n3)cc2)cc1 ; c1ccc(-c2ccc(-c3nc(-c4ccc(-c5ccccc5)cc4)nc(-c4cccc5sc6ccccc6c45)n3)cc2)cc1 ; c1ccc(-c2ccc(-c3nc(-c4cccc(-c5ccccc5)c4)nc(-c4cccc5sc6ccccc6c45)n3)cc2)cc1]

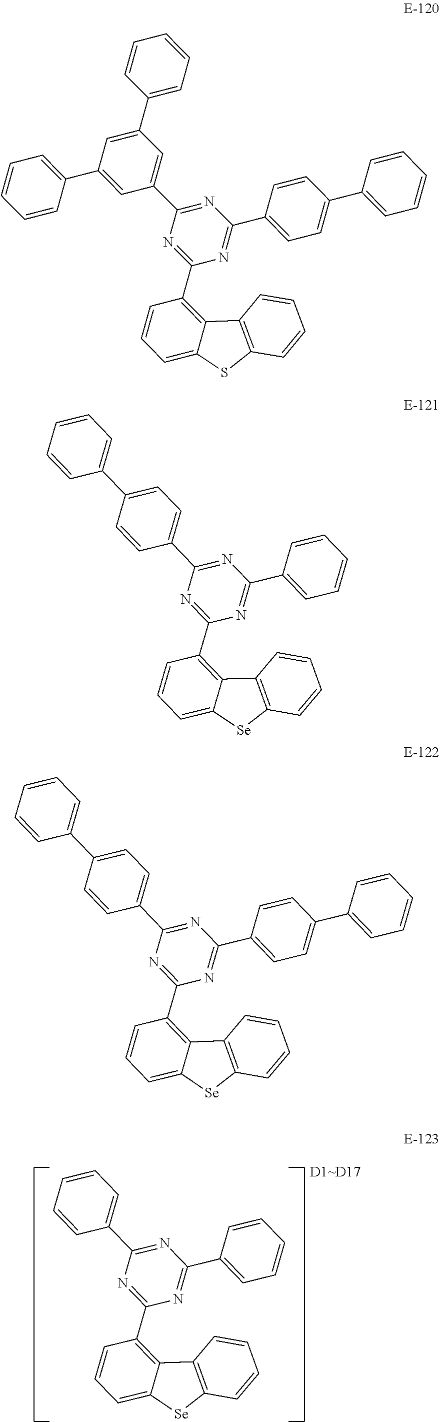

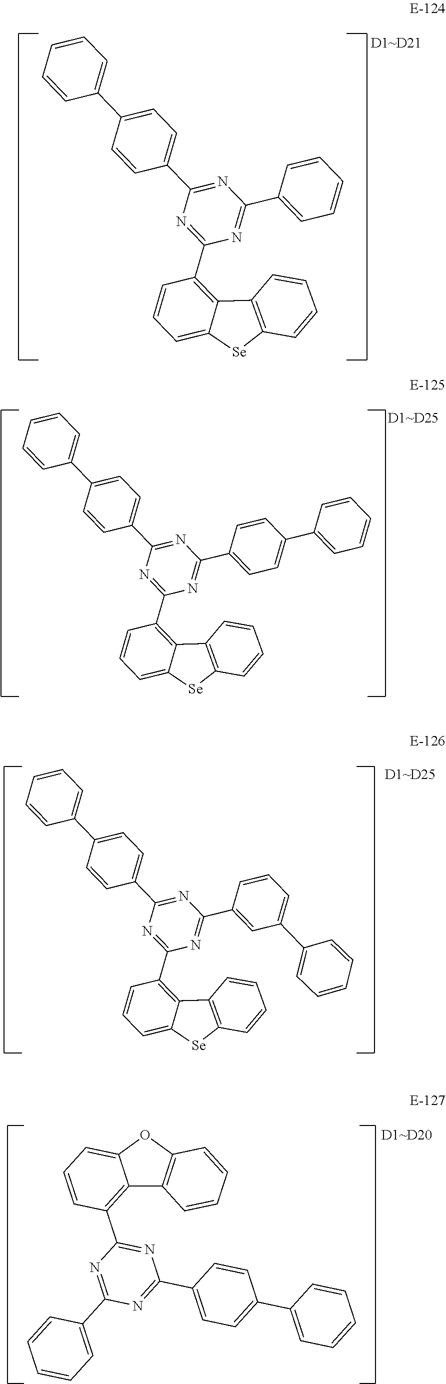

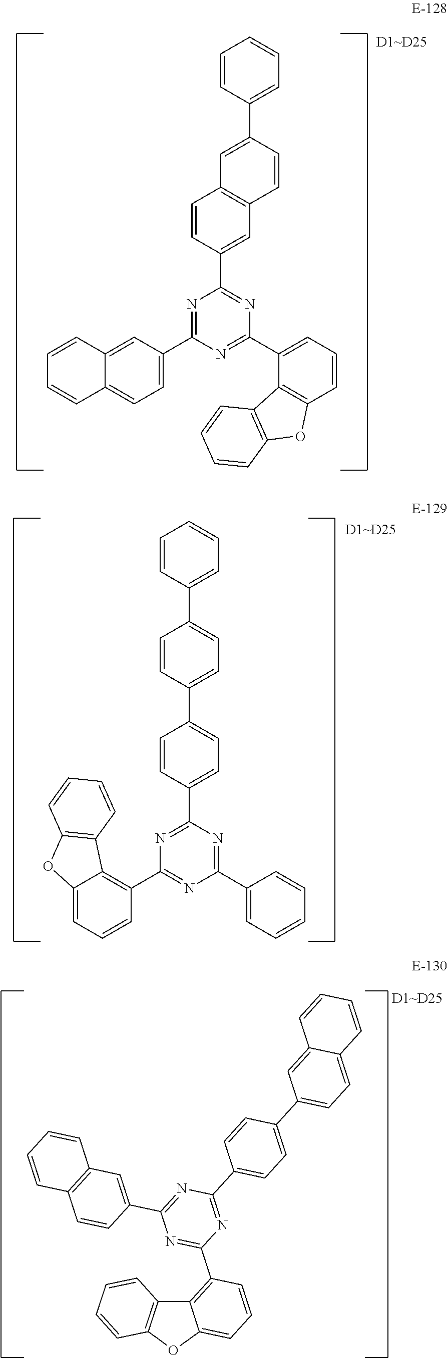

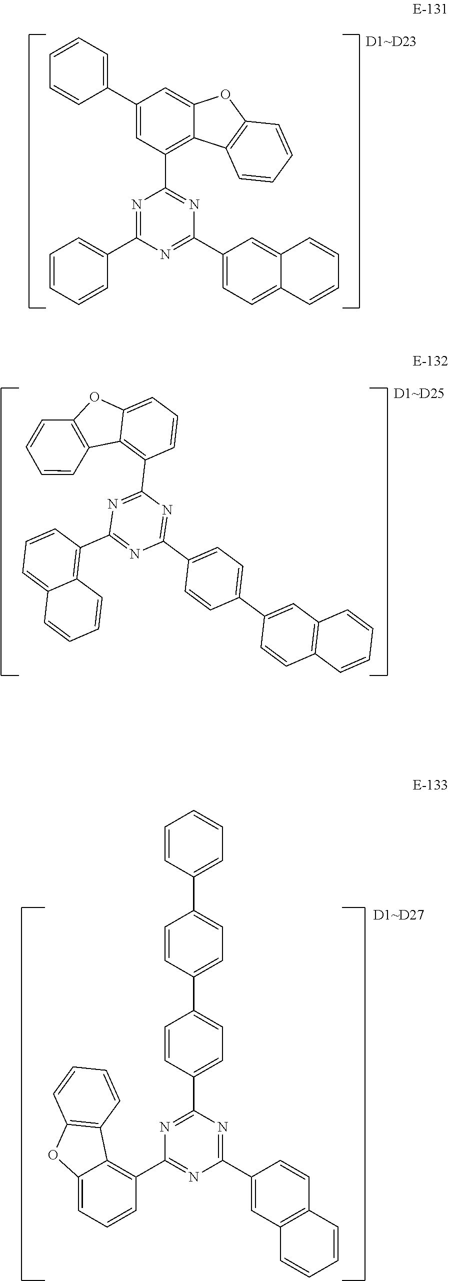

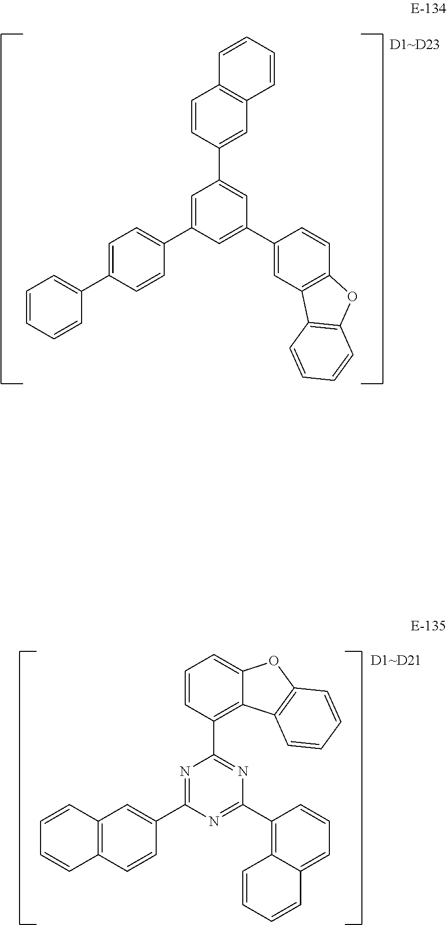

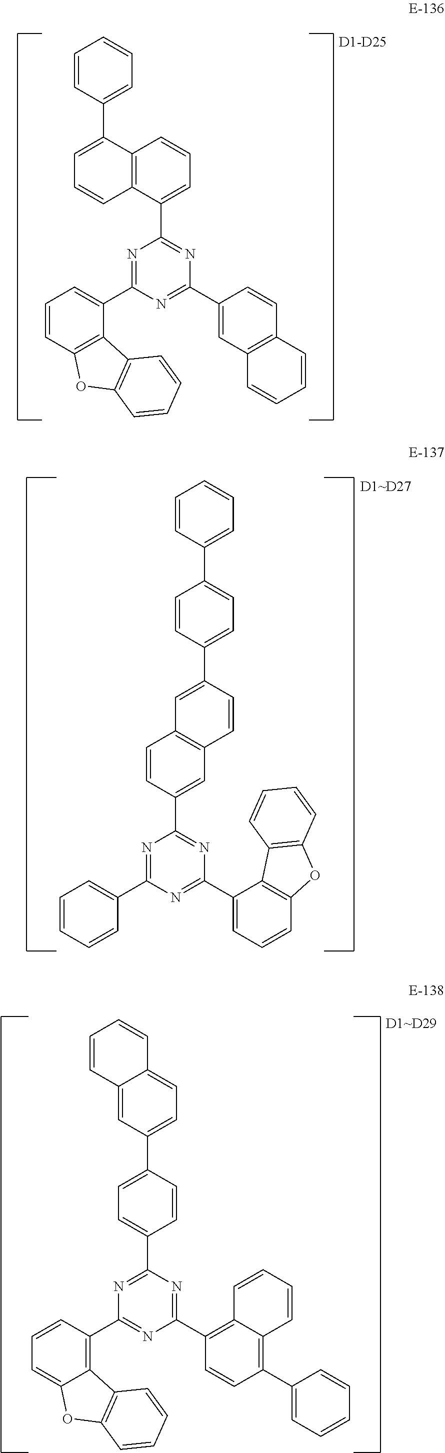

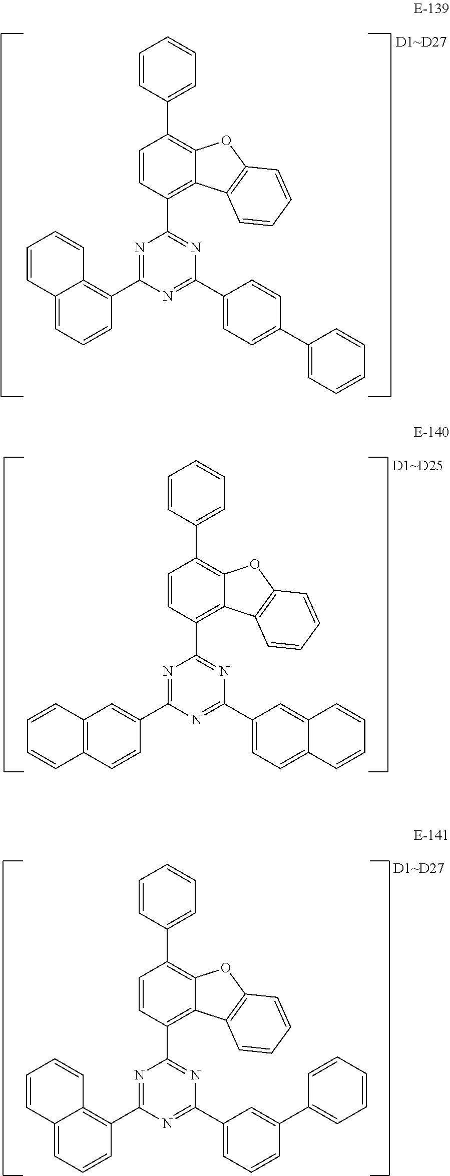

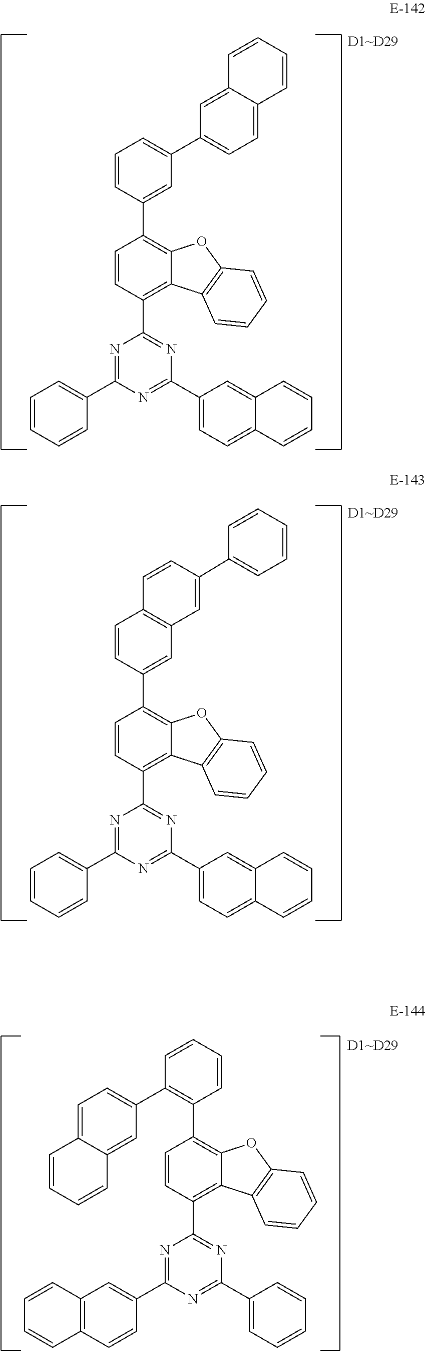

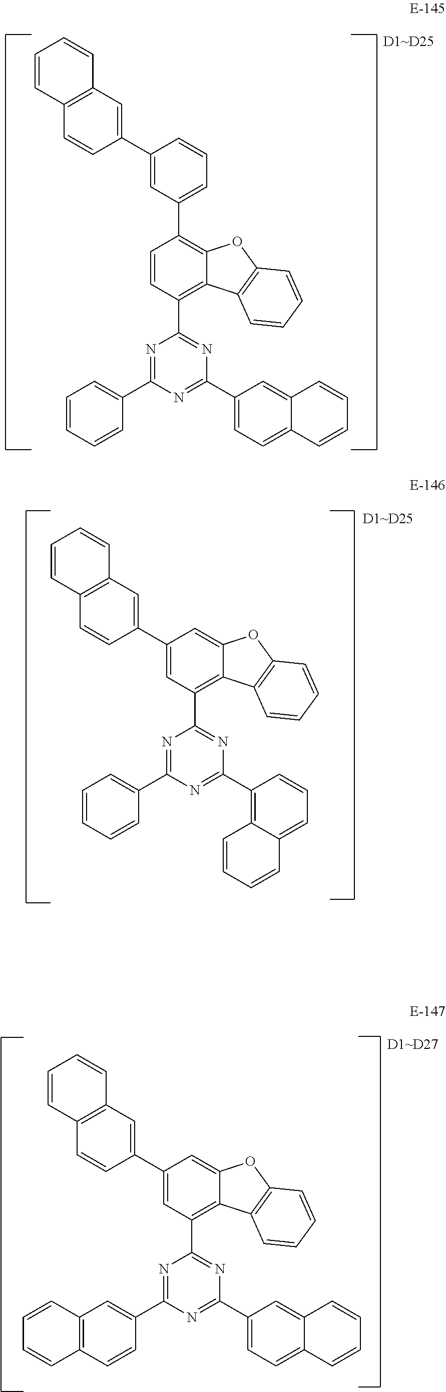

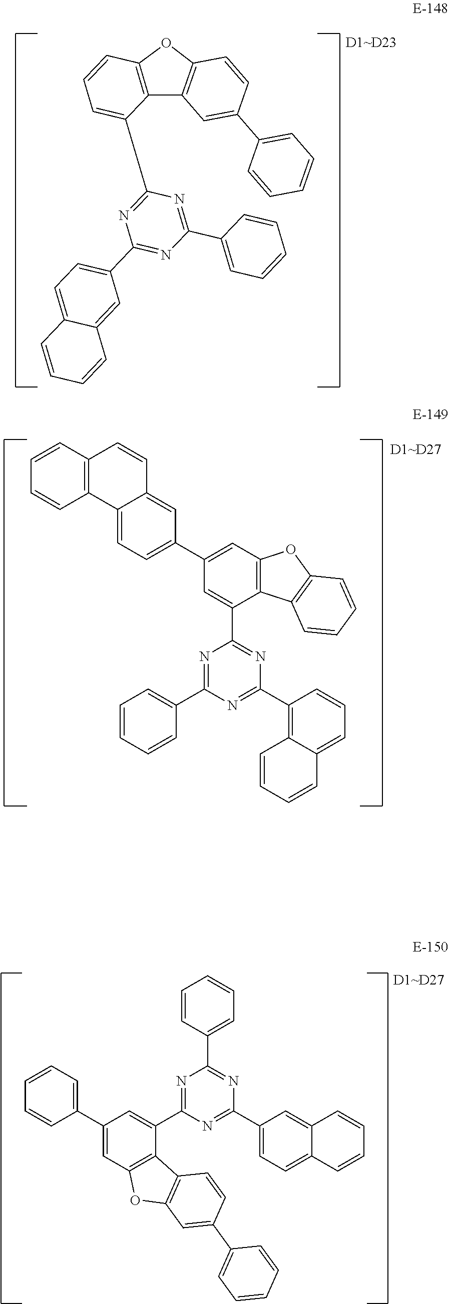

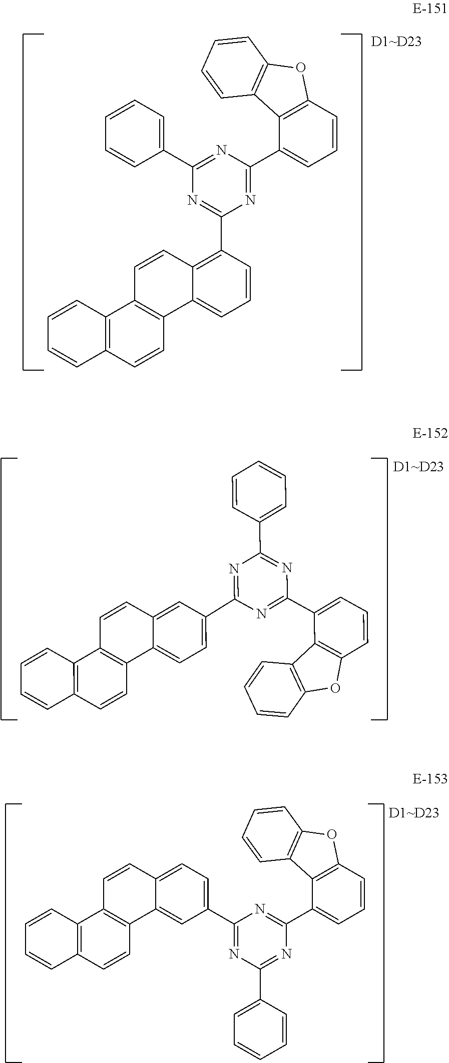

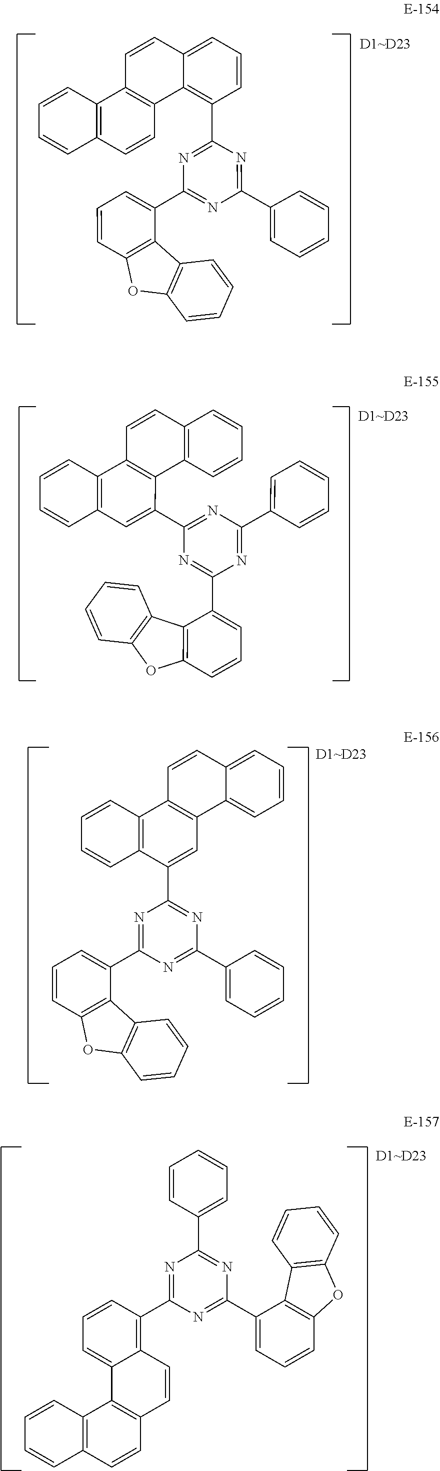

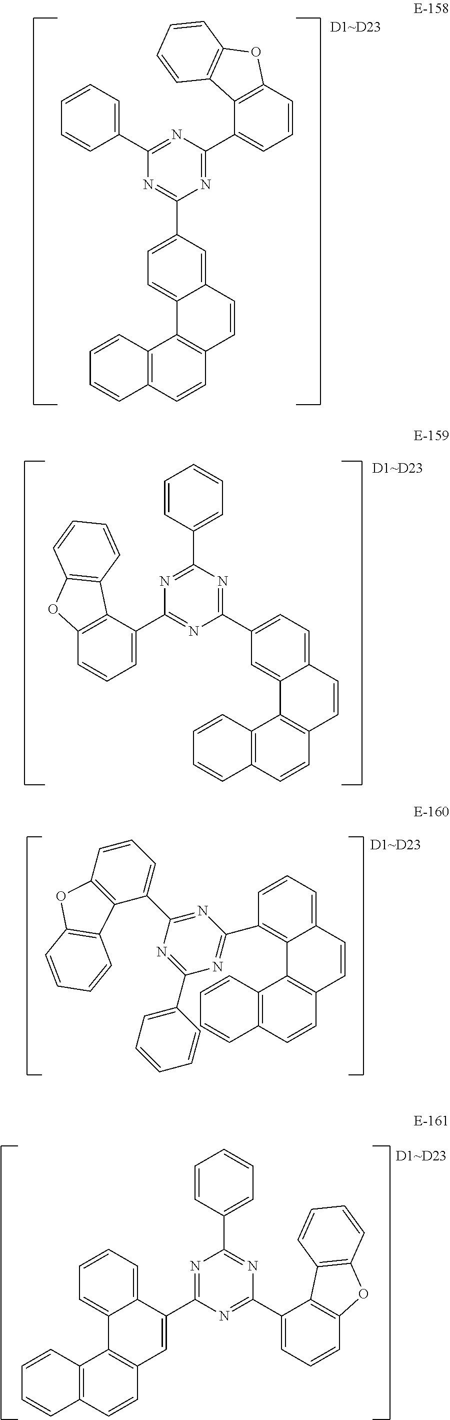

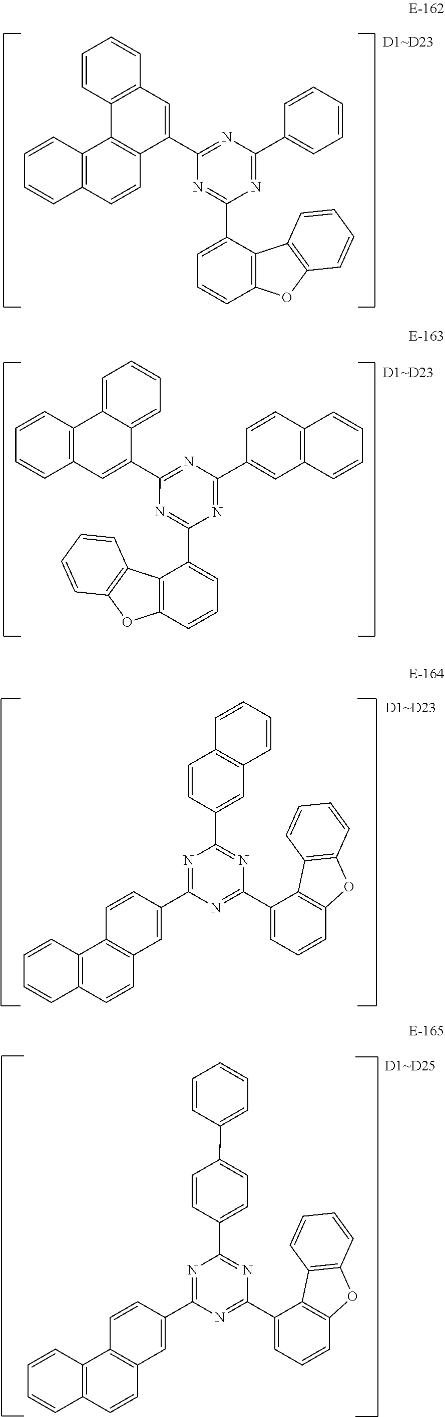

In the compounds above, n In Dn means the number of deuteriums that can be substituted in the compound.

The compound represented by Formula 2 according to the present disclosure may be prepared by a synthetic method known to a person skilled in the art.

Hereinafter, an organic electroluminescent device to which the aforementioned plurality of host materials is applied to will be described.

The organic electroluminescent device according to one embodiment includes a first electrode; a second electrode; and at least one organic layer(s) interposed between the first electrode and the second electrode. The organic layer may include a light-emitting layer, and the light-emitting layer may comprise a plurality of host materials comprising at least one first host compound represented by Formula 1 and at least one second host compound represented by Formula 2. Wherein, the weight ratio of the first host compound to the second host compound may be in the range of about 1:99 to about 99:1, preferably about 10:90 to about 90:10, more preferably about 30:70 to about 70:30, more preferably about 40:60 to about 60:40, and even more preferably about 50:50 in the light-emitting layer.

According to one embodiment, the plurality of host materials of the present disclosure comprises at least one compound(s), which is a first host compound, and at least one compound(s), which is a second host compound. The plurality of host materials may be included in the same organic layer, for example the same light-emitting layer, or may be included in different light-emitting layers.

The organic layer may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer and an electron buffer layer, in addition to the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Further, the electron transport layer, the electron injection layer, the electron buffer layer, or the hole blocking layer may contain the azine-based compound as an electron transport material, an electron injection material, an electron buffer material, or a hole blocking material. Further, the organic layer may further comprise at least one metal selected from the group consisting of metals from Group 1, metals from Group 2, transition metals of the 4^{th} period, transition metals of the 5^{th} period, lanthanides, and organic metals of the d-transition elements of the Periodic Table, or at least one complex compound comprising such a metal.

The plurality of host materials according to one embodiment may be used as light-emitting materials for a white organic light-emitting device. The white organic light-emitting device has suggested various structures, such as a parallel side-by-side arrangement method, a stacking arrangement method, CCM (color conversion material) method, etc., according to the arrangement of R (Red), G (Green), YG (yellowish-green), or B (blue) light-emitting units. In addition, the plurality of host materials according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a dual-side emission type according to the kinds of the material forming the first electrode and the second electrode.

A hole injection layer, a hole transport layer, an electron blocking layer, or a combination thereof can be used between the anode and the light-emitting layer. The hole injection layer may be multi-layered in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layered, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layered in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layered, wherein each layer may use a plurality of compounds. Further, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting hole injection and/or hole transport, or for preventing the overflow of electrons. When the light-emitting auxiliary layer is placed between the cathode and the light-emitting layer, it can be used for promoting the electron injection and/or electron transport, or for preventing the hole overflow. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer which is further included may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X}(1≤X≤2), AlO_{X}(1≤X≤1.5), SiON, SiAlON, etc.; the halogenated metal includes LiF, MgF₂, CaF₂, a rare earth metal fluoride, etc.; and the metal oxide includes CS₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation; thus, it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Further, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto:

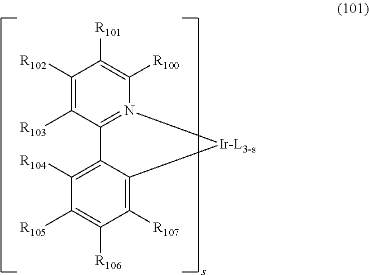

In Formula 101,
L is any one selected from the following structures 1 to 3;

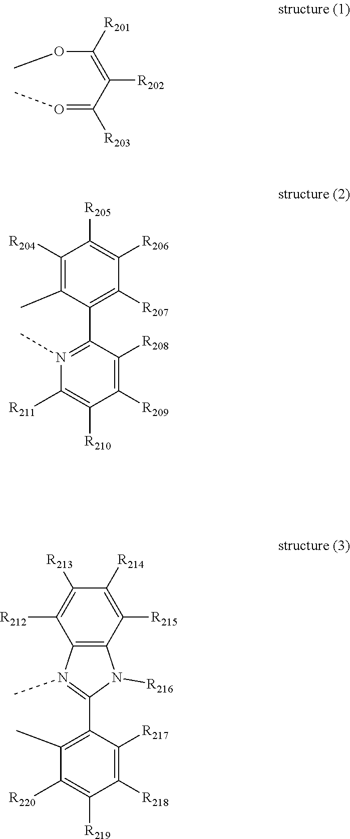

in structures 1 to 3,
R₁₀₀ to R₁₀₃ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3-to 30-membered)heteroaryl, or a substituted or unsubstituted (C1-C30)alkoxy; or may be linked to the adjacent substituents to form a ring(s), for example, to form a ring(s) with a pyridine such as a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;

R₁₀₄ to R₁₀₇ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3-to 30-membered)heteroaryl, cyano, or a substituted or unsubstituted (C1-C30)alkoxy; or may be linked to an adjacent substituent(s) to form a substituted or unsubstituted ring(s), for example, to form a substituted or unsubstituted ring(s) with a benzene such as a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent, hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or may be linked to an adjacent substituent(s) to form a substituted or unsubstituted ring(s); and
s represents an integer of 1 to 3.

Specifically, the specific examples of the dopant compound include the following, but are not limited thereto:

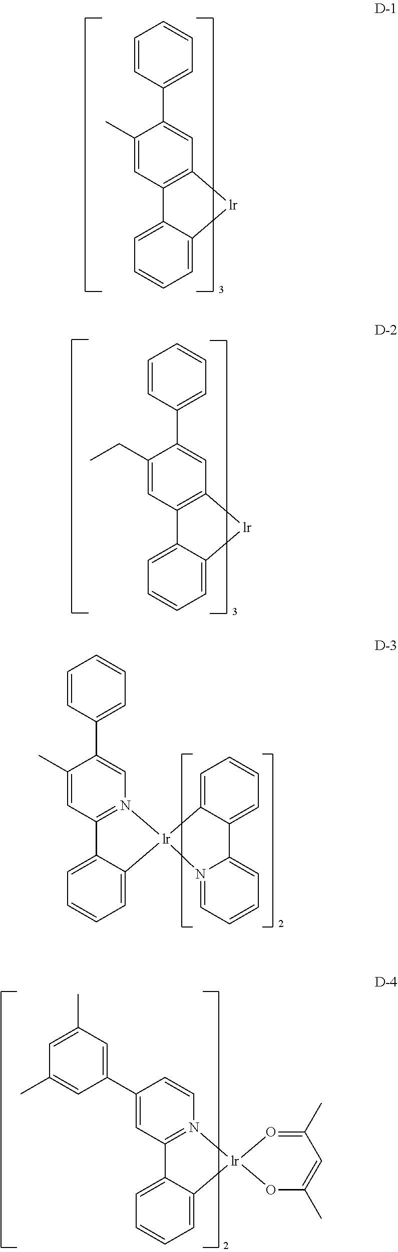

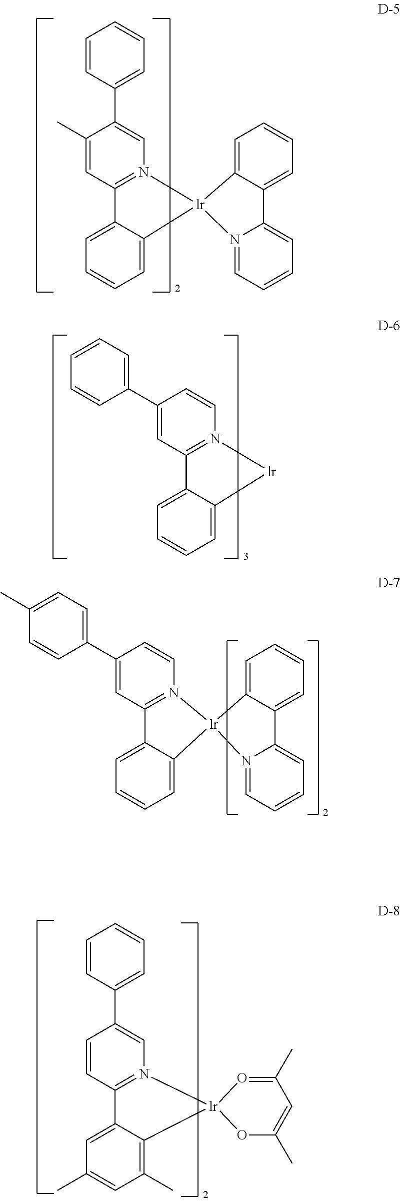

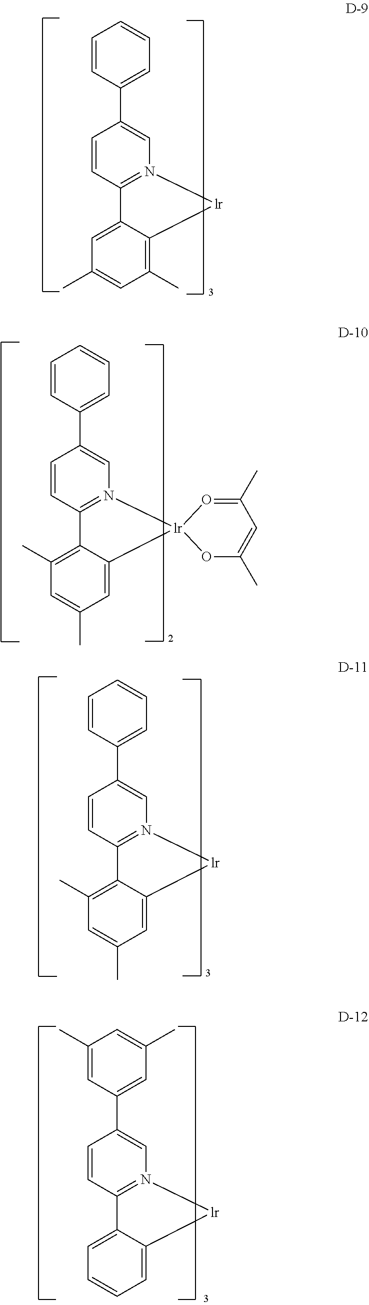

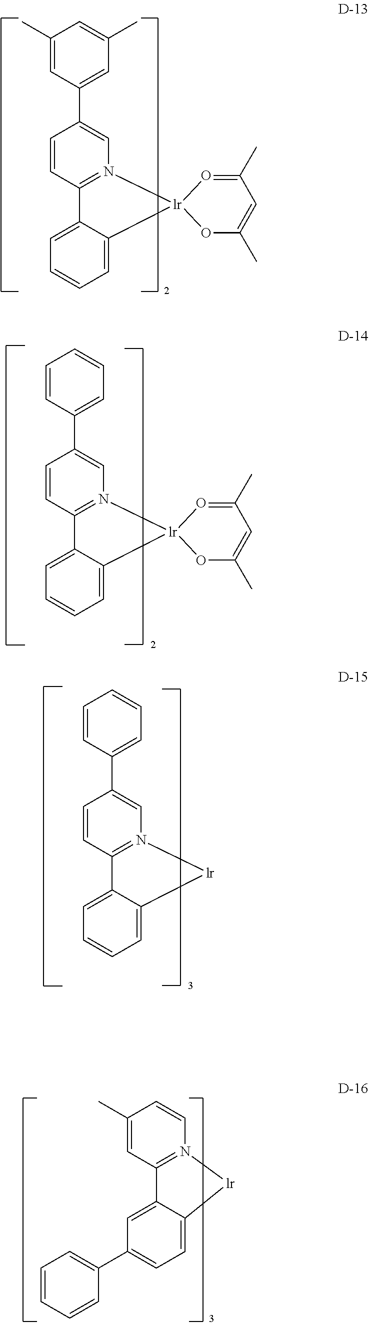

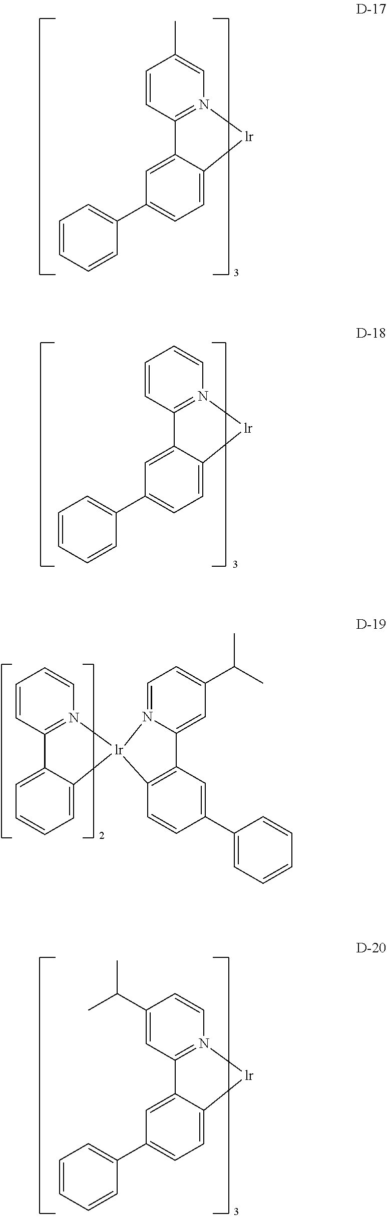

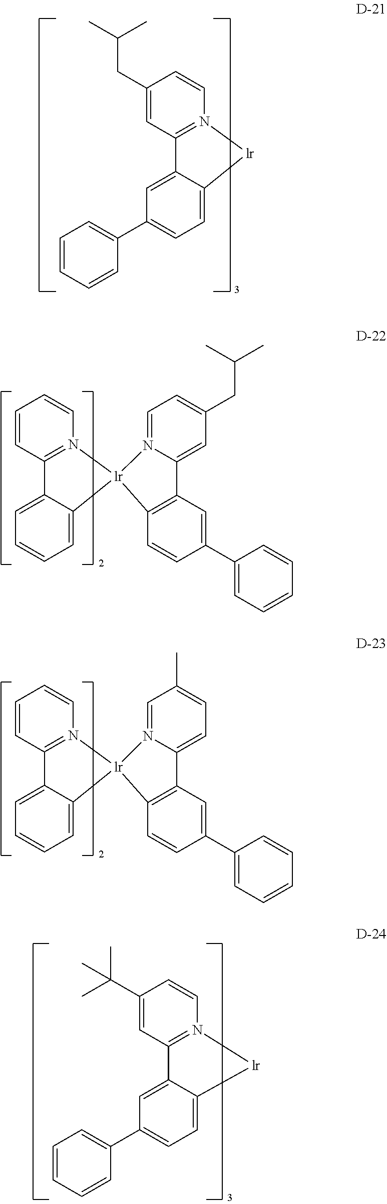

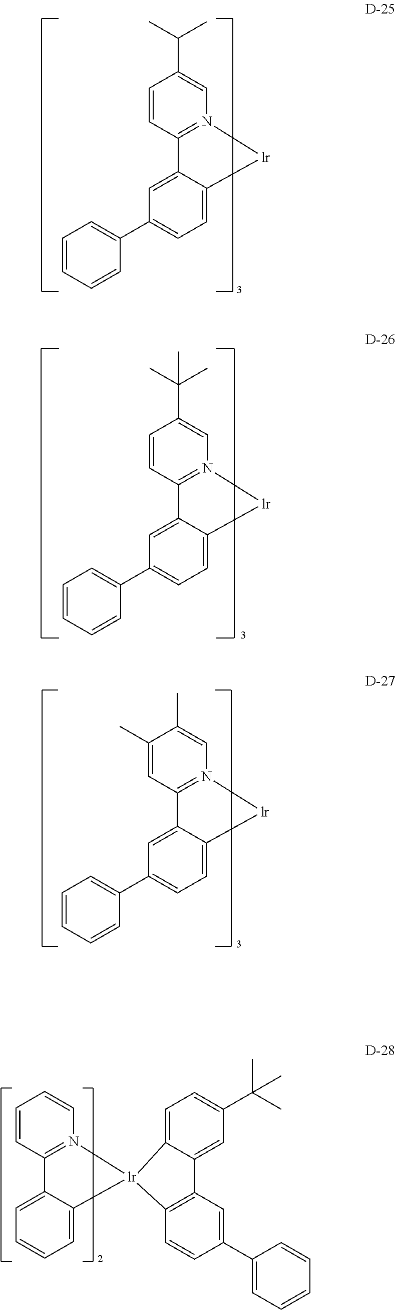

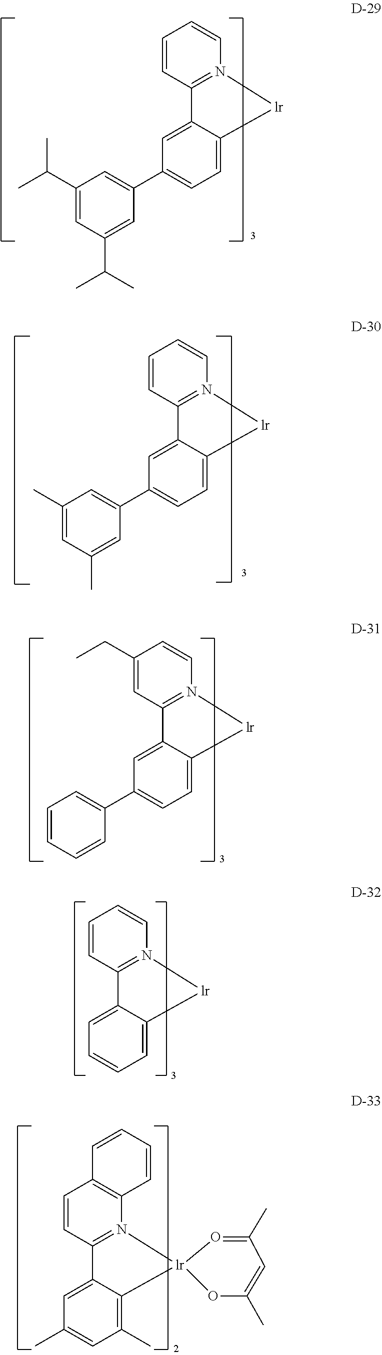

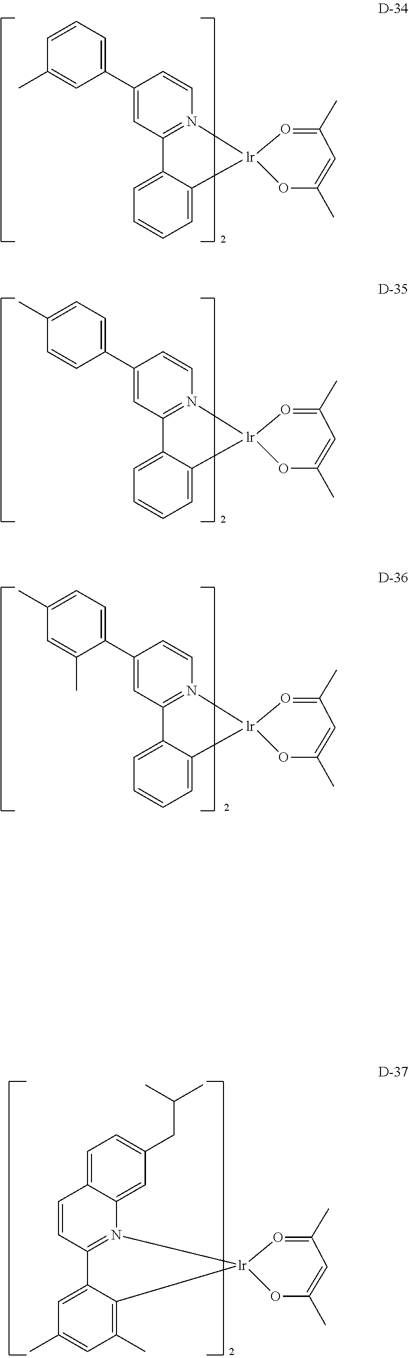

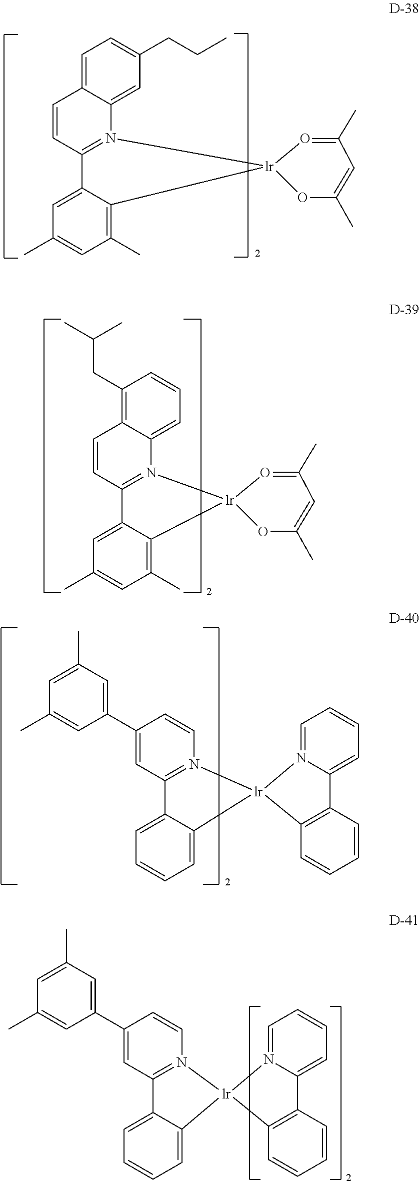

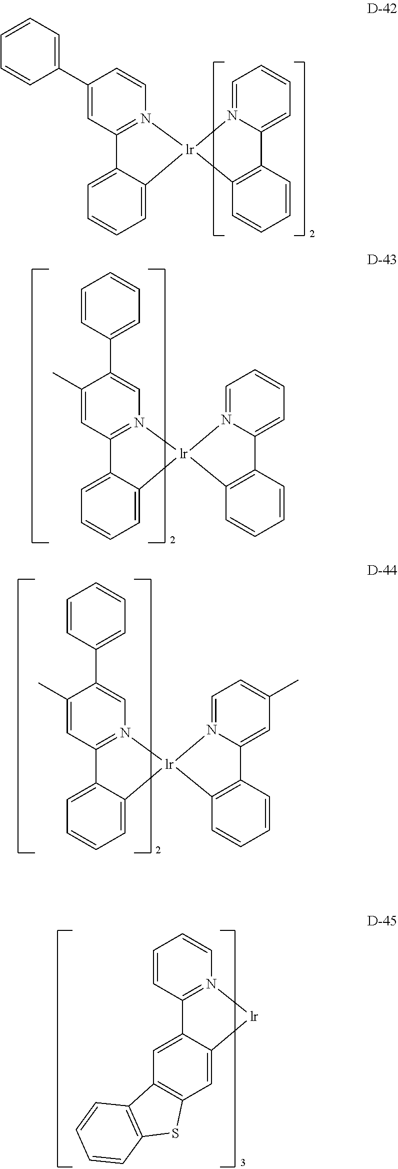

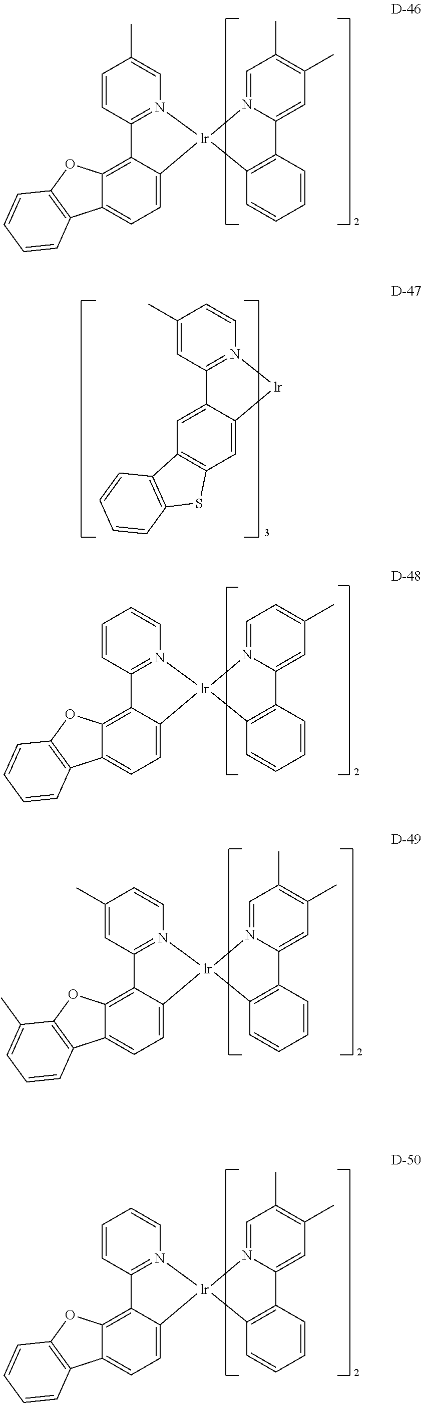

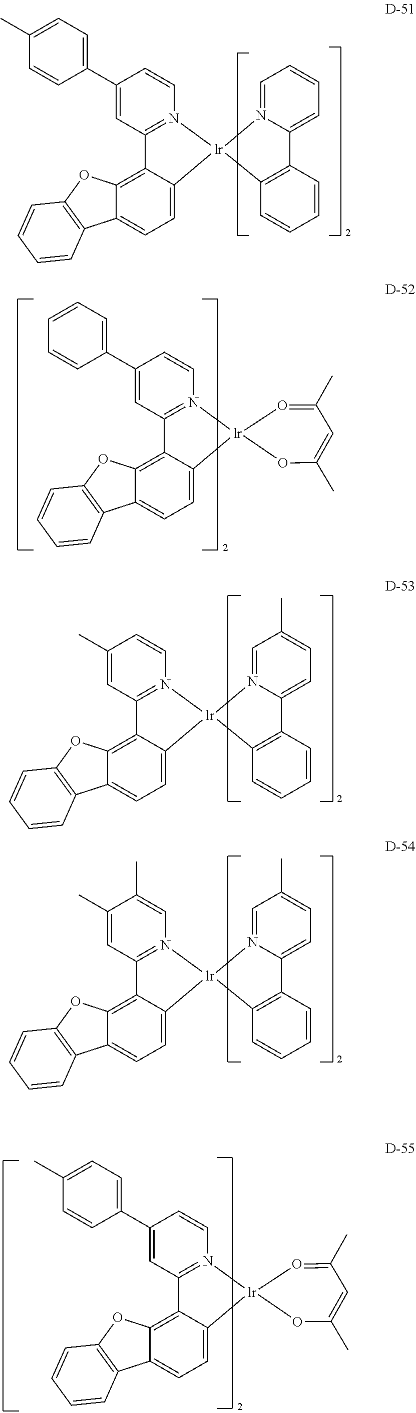

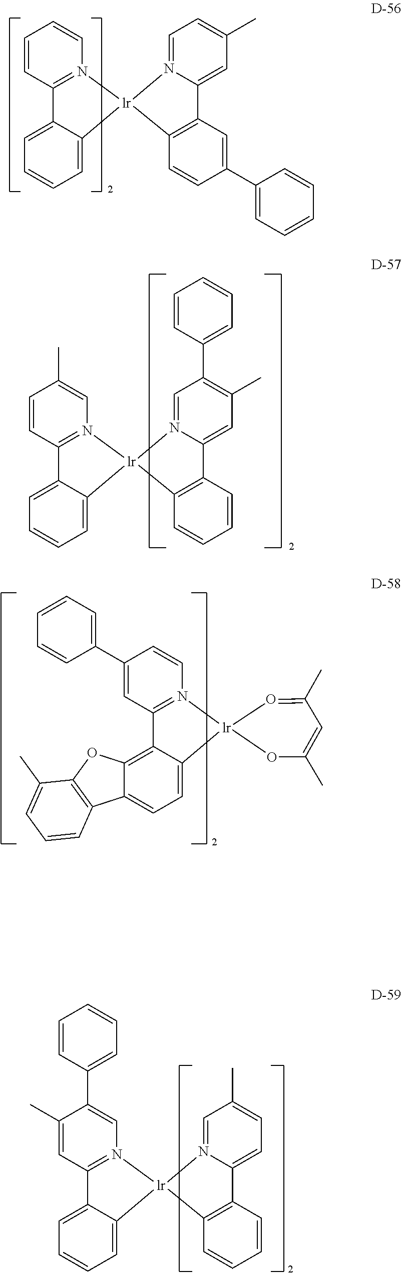

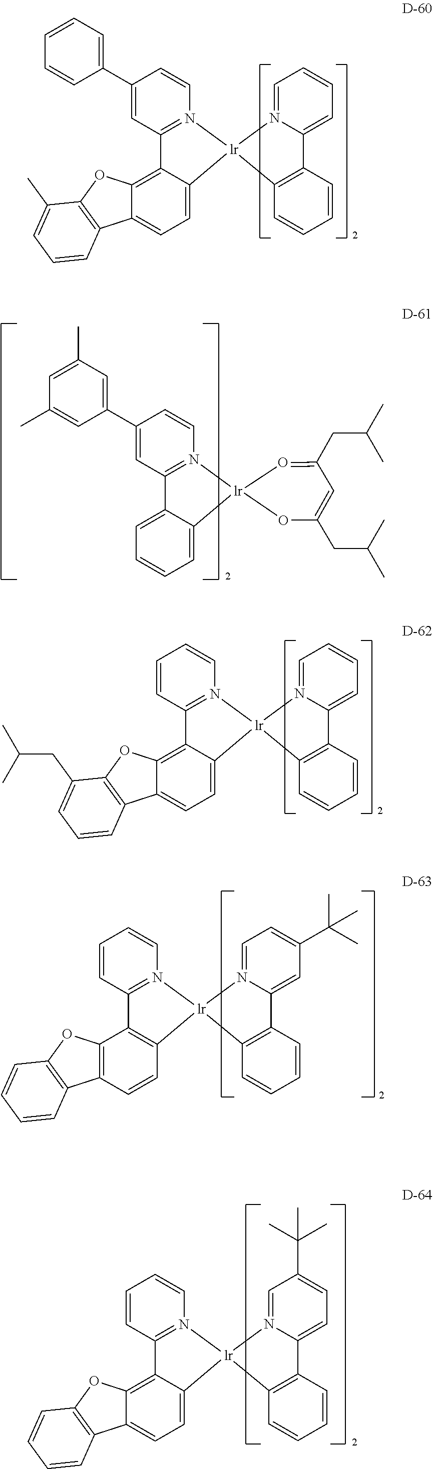

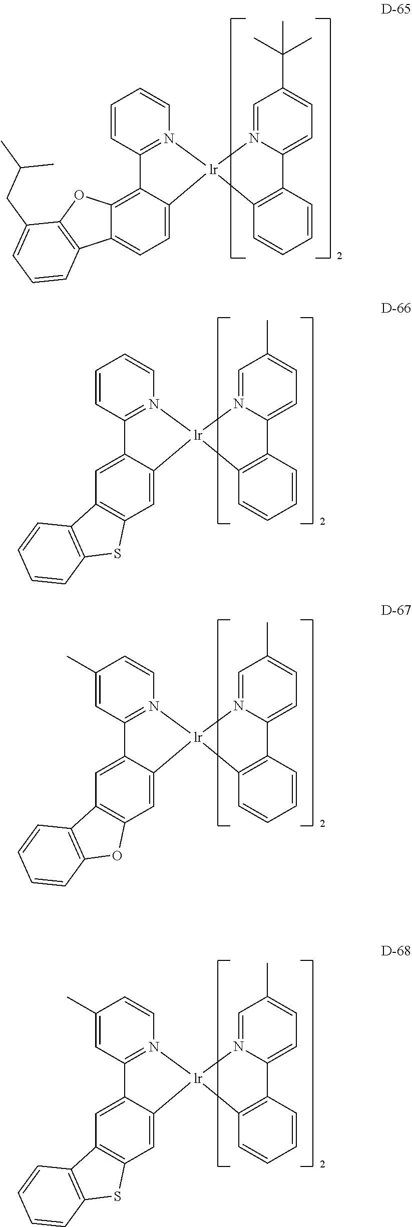

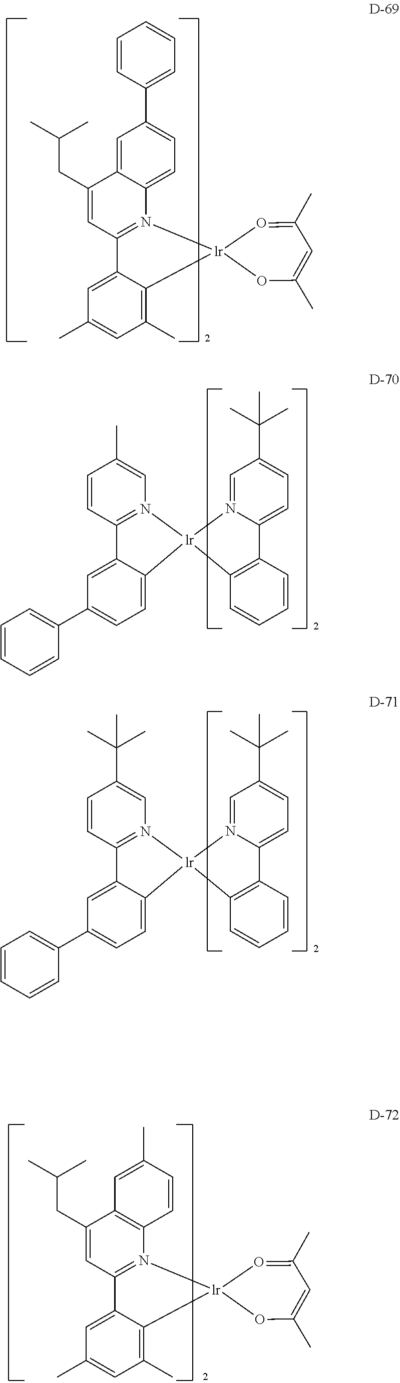

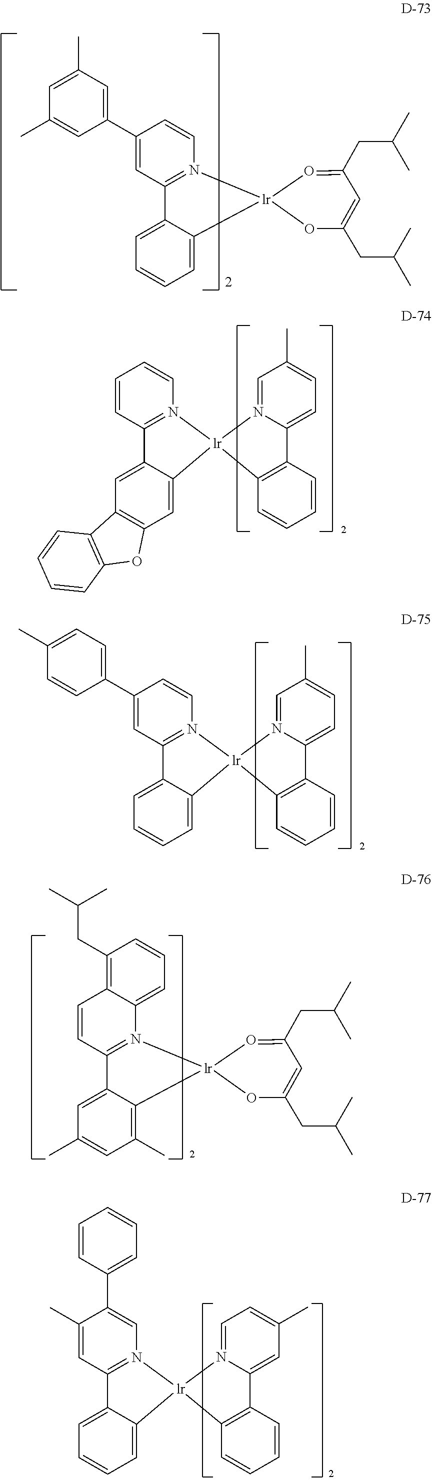

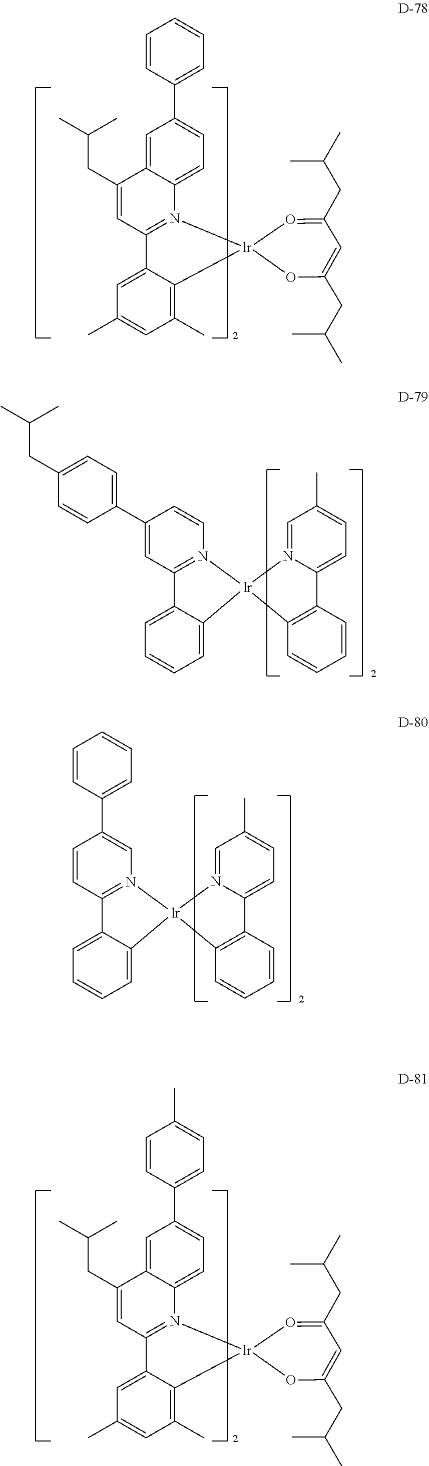

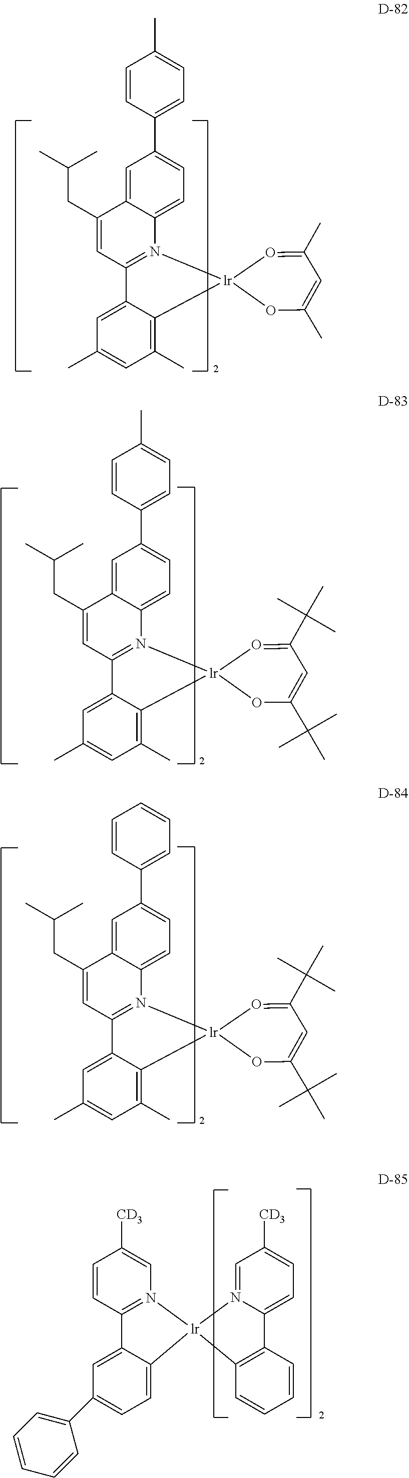

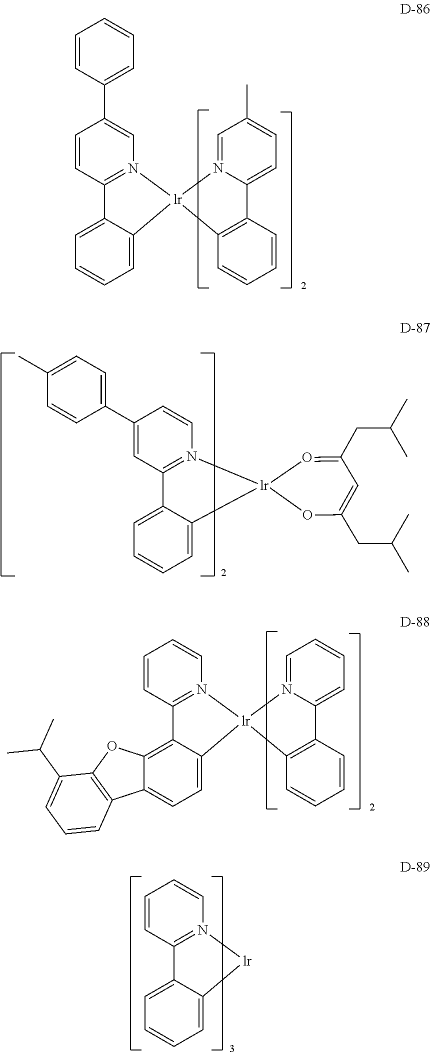

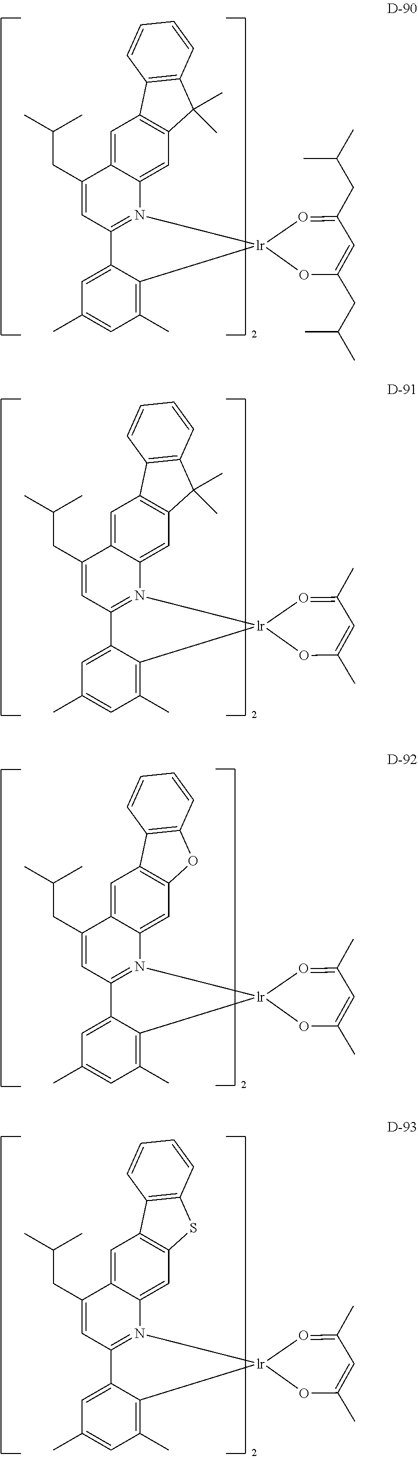

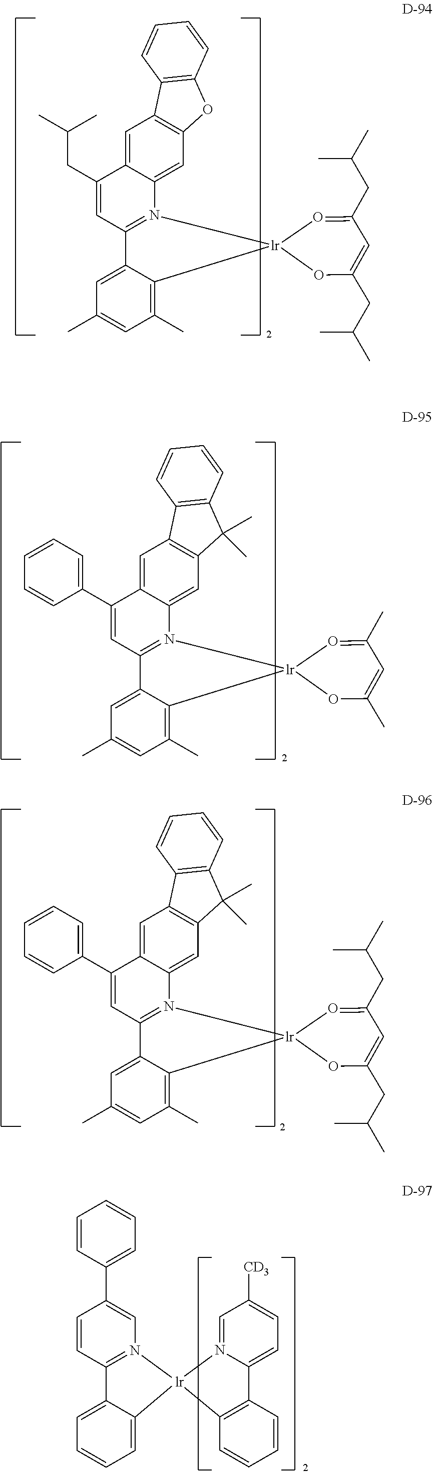

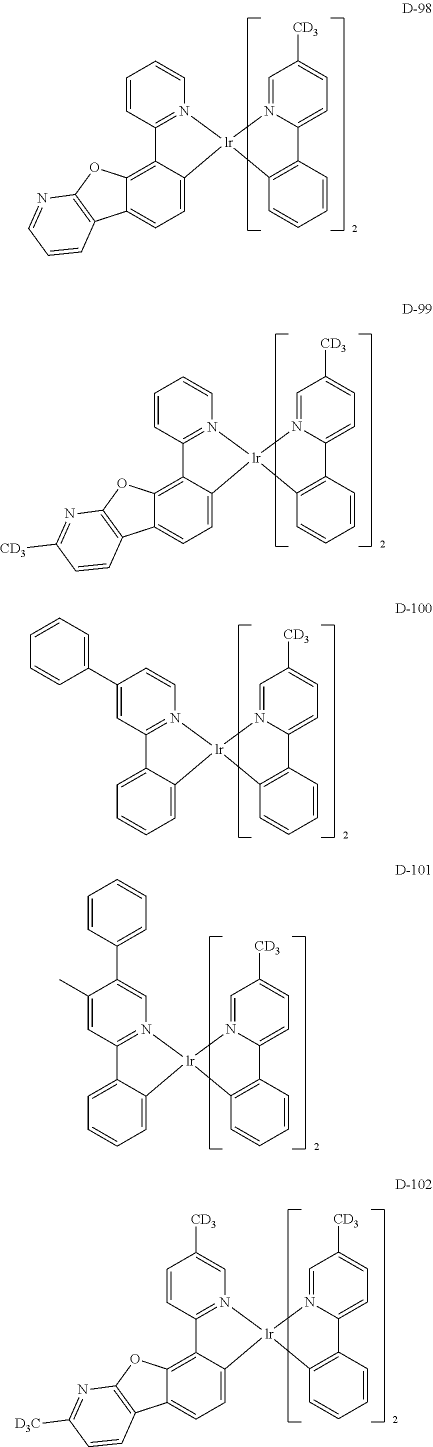

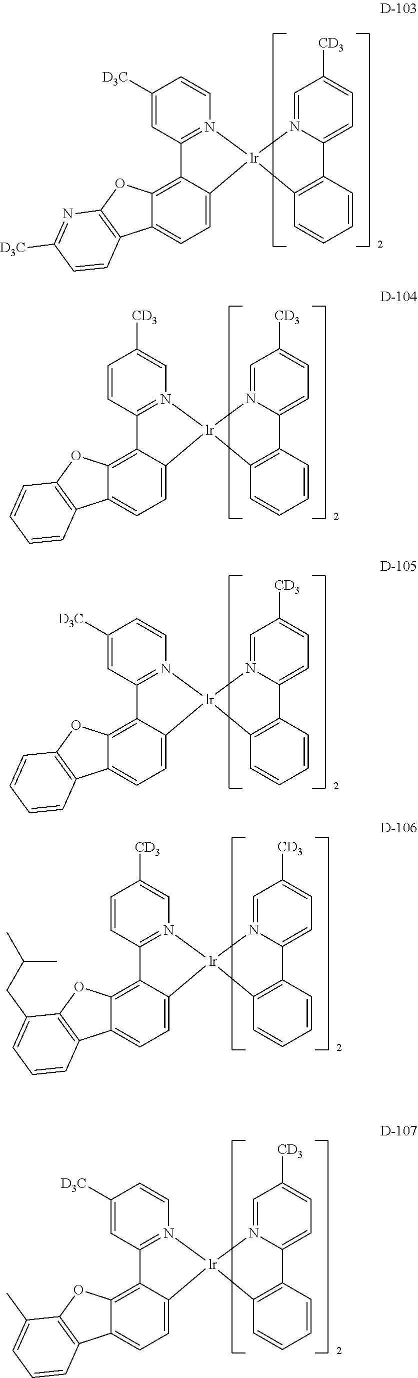

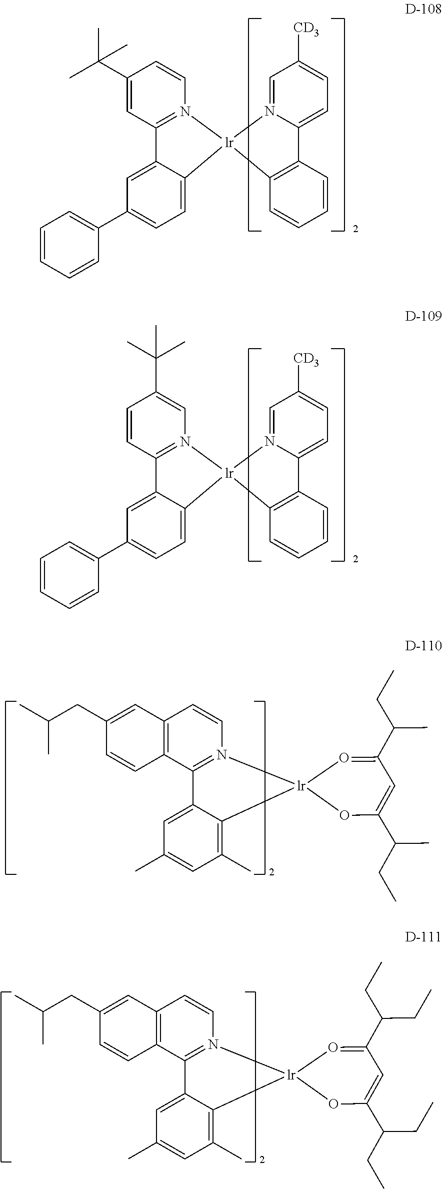

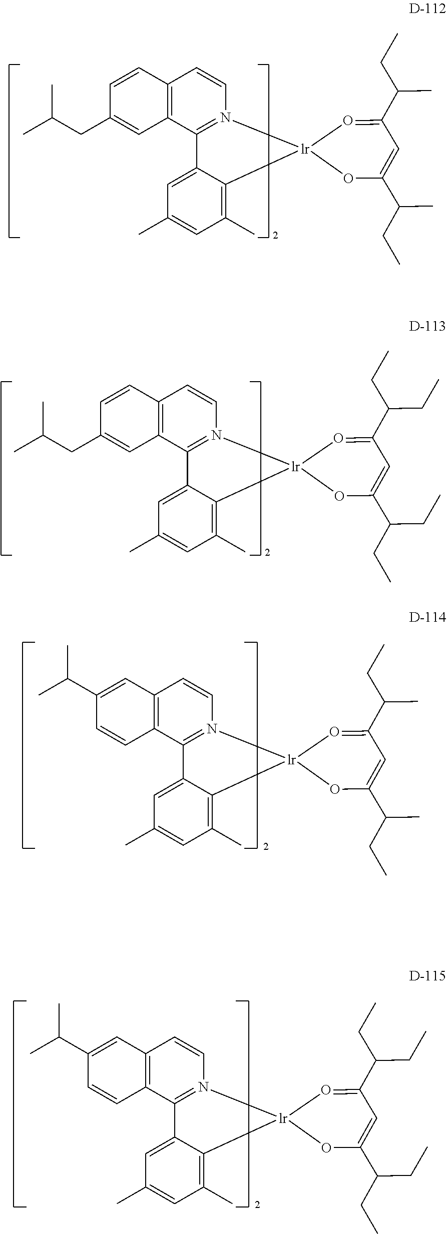

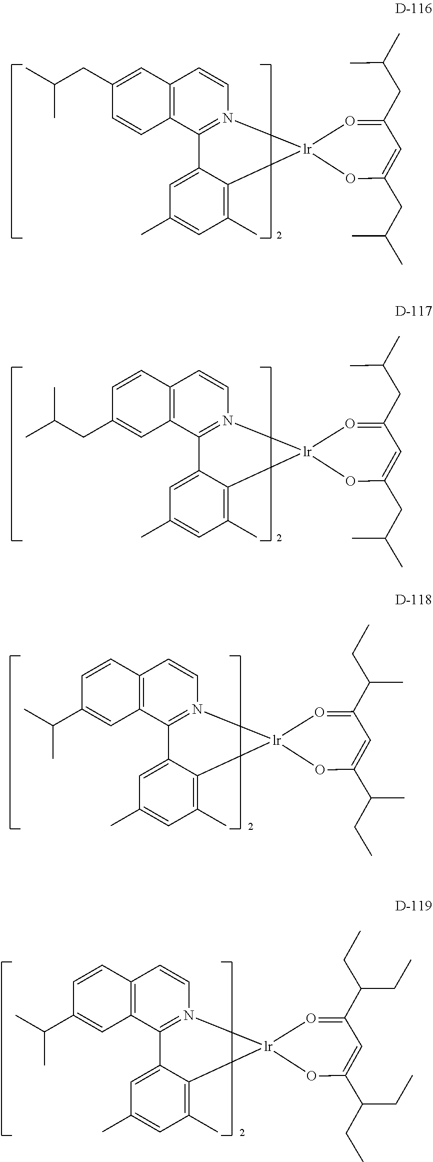

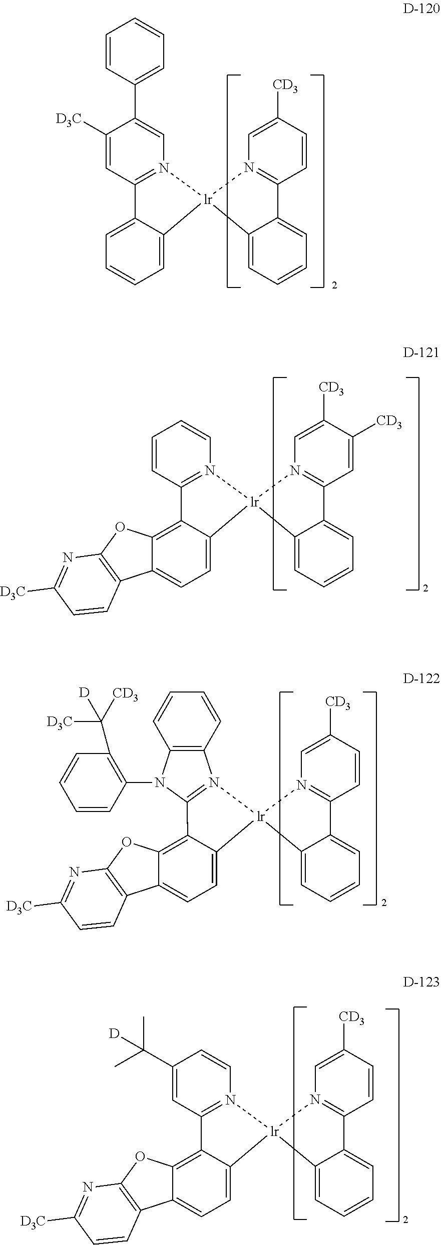

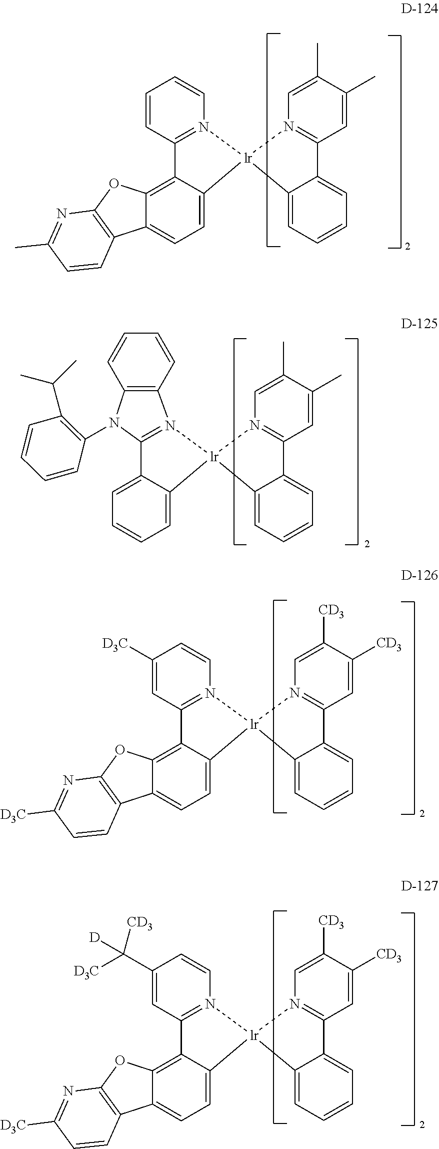

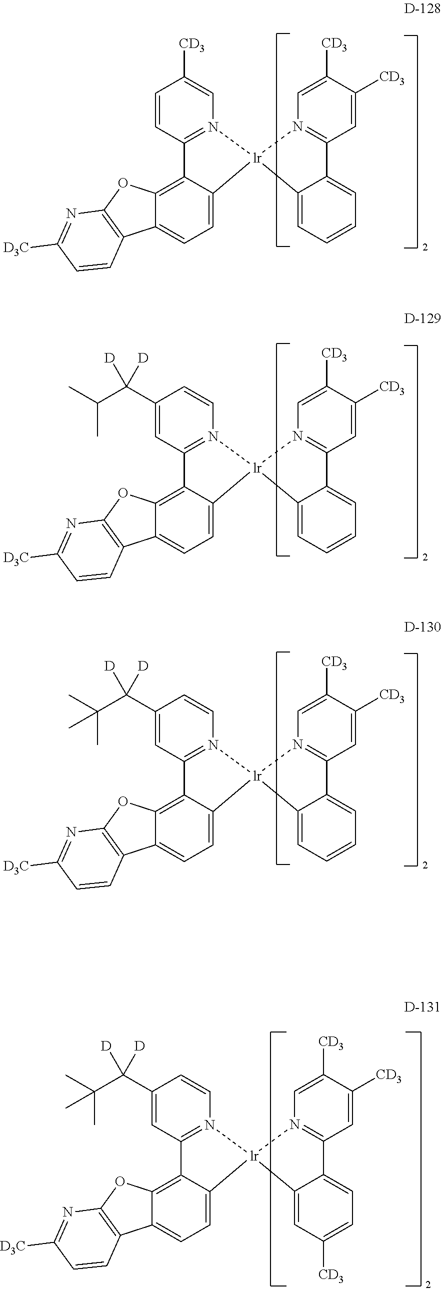

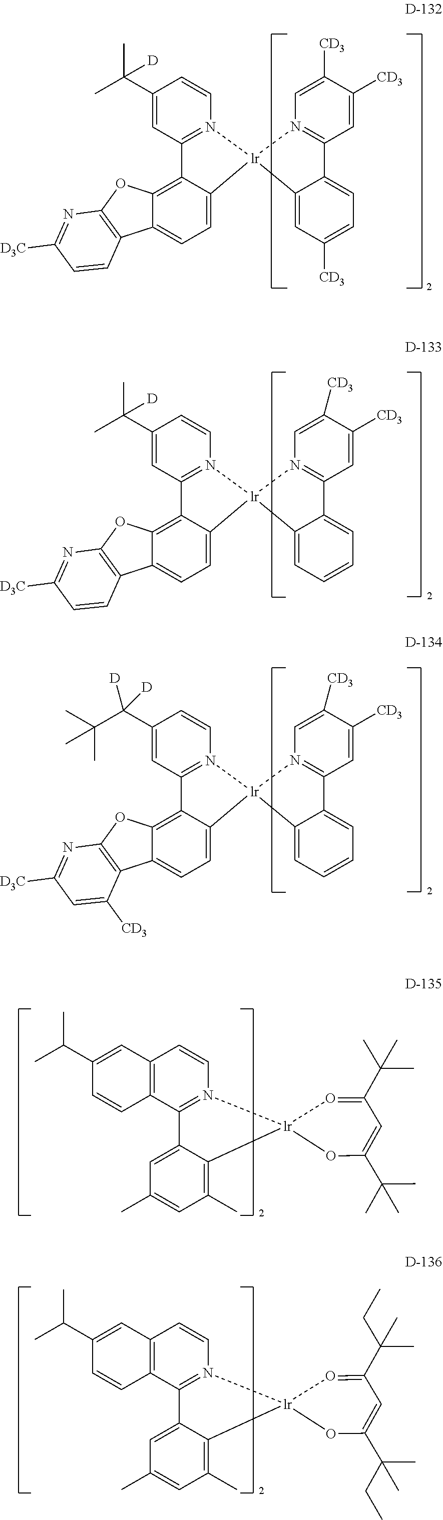

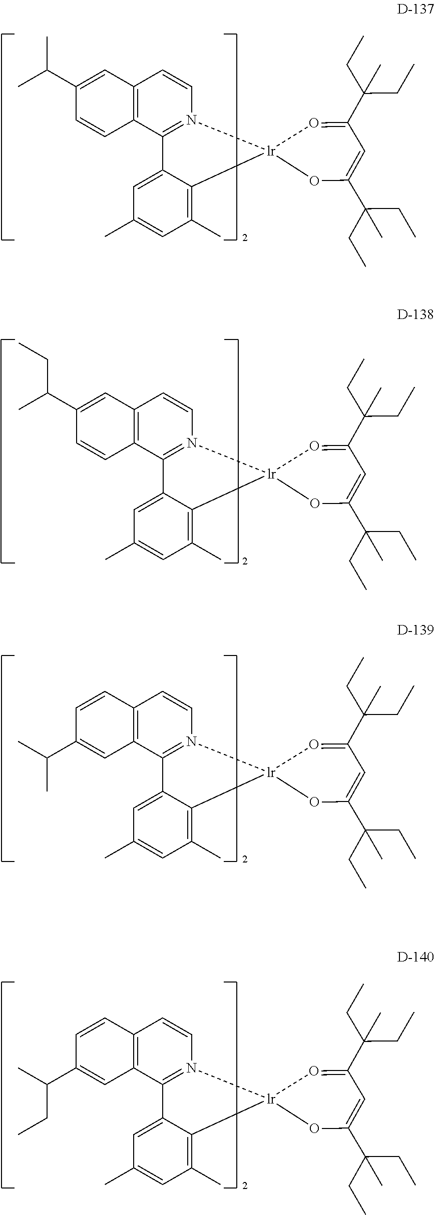

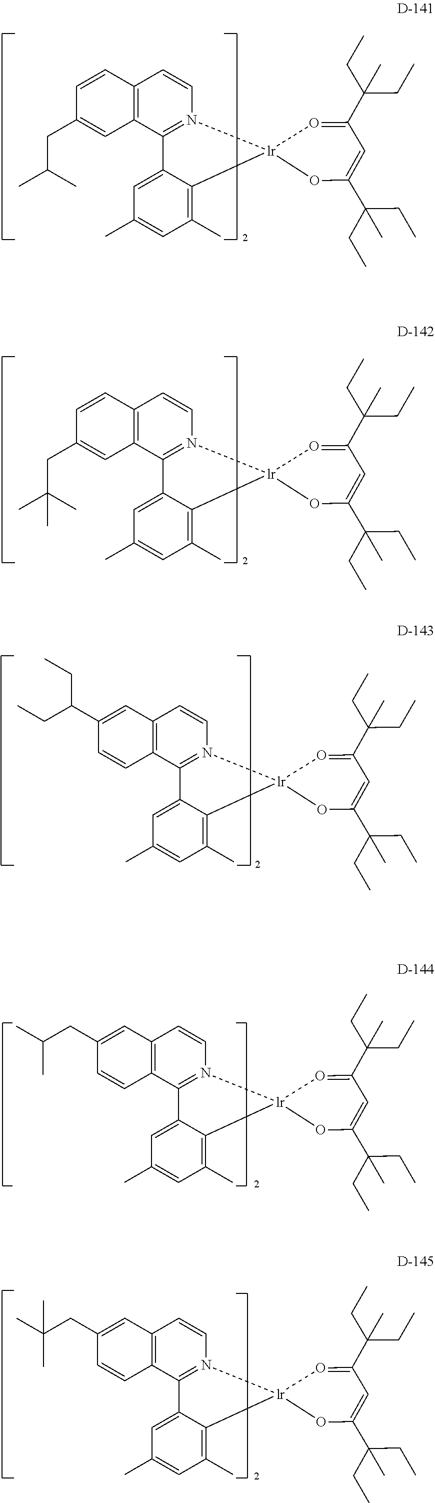

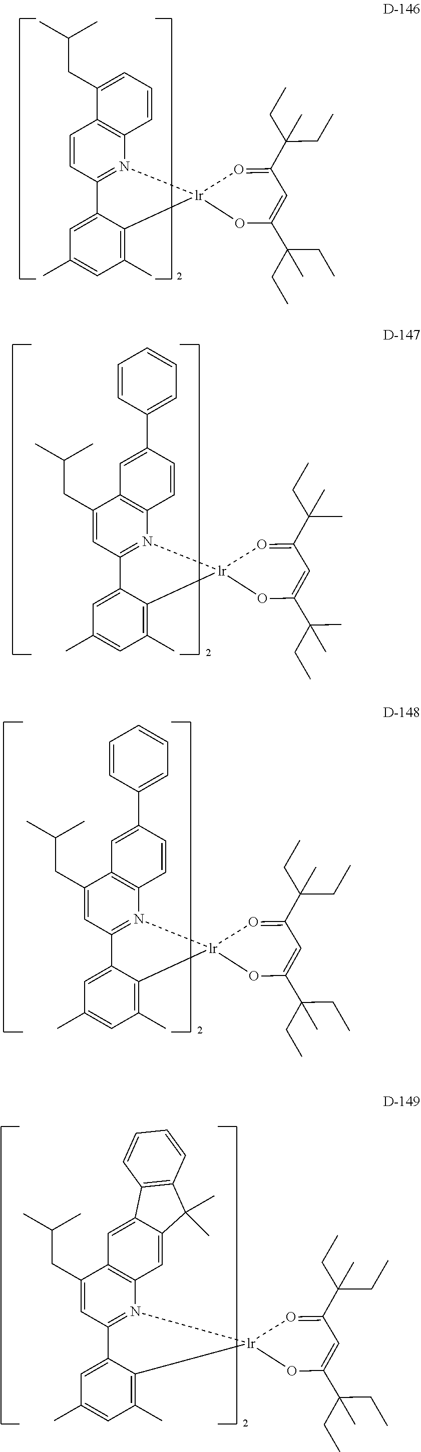

In order to form each layer of the organic electroluminescent device of the present disclosure, dry film-forming methods such as vacuum evaporation, sputtering, plasma, ion plating methods, etc., or wet film-forming methods such as spin coating, dip coating, flow coating methods, etc., can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host compound and the second host compound according to one embodiment, the layer can be formed by the above-listed methods, and can often be formed by co-deposition or mixture-deposition. Co-deposition is a mixed deposition method in which two or more materials are put into respective individual crucible sources and a current is applied to both cells simultaneously to evaporate the materials and to perform mixed deposition; mixed deposition is a method in which two or more materials are mixed in one crucible source before deposition, and then a current is applied to one cell to evaporate the materials.

According to one embodiment, when the first host compound and the second host compound exist in the same layer or different layers in the organic electroluminescent device, the layers by the two host compounds may be separately formed. For example, after depositing the first host compound, a second host compound may be deposited.

According to one embodiment, the present disclosure can provide display devices comprising a plurality of host materials comprising a first host compound represented by Formula 1 and a second host compound represented by Formula 2. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method for the host materials according to the present disclosure will be explained with reference to the synthesis method of a representative compound or intermediate compound in order to understand the present disclosure in detail.

### EXAMPLE 1

### Synthesis of Compound H-94

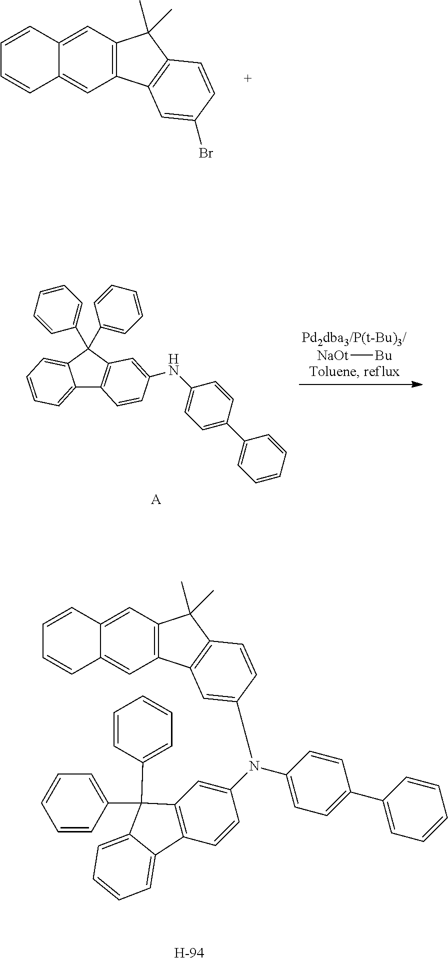

3-Bromo-11,11-dimethyl-11H-benzo[b]fluorene (13 g, 42 mmol), Compound A (17.5 g, 42 mmol), tris(dibenzylideneacetone)dipalladium(0) (Pd₂(dba)₃) (1.2 g, 1.26 mmol), tri-t-butyl phosphine (P(t-Bu)₃) (50%) (1.2 mL, 2.52 mmol), sodium tert-butoxide (NaOt-Bu) (6 g, 63 mmol), and 210 mL of toluene were added to the reaction vessel and stirred under reflux at 140° C. for 4 hours. When the reaction was completed, the reaction mixture was cooled to room temperature and extracted with H₂O plus ethyl acetate. Next, it was distilled under reduced pressure and purified by column chromatography to obtain Compound H-94 (15 g, yield: 49%).

| | MW | M.P. |
|---|---|---|
| H-94 | 727.95 | 180° C. |

### EXAMPLE 2

### Synthesis of Compound H-127

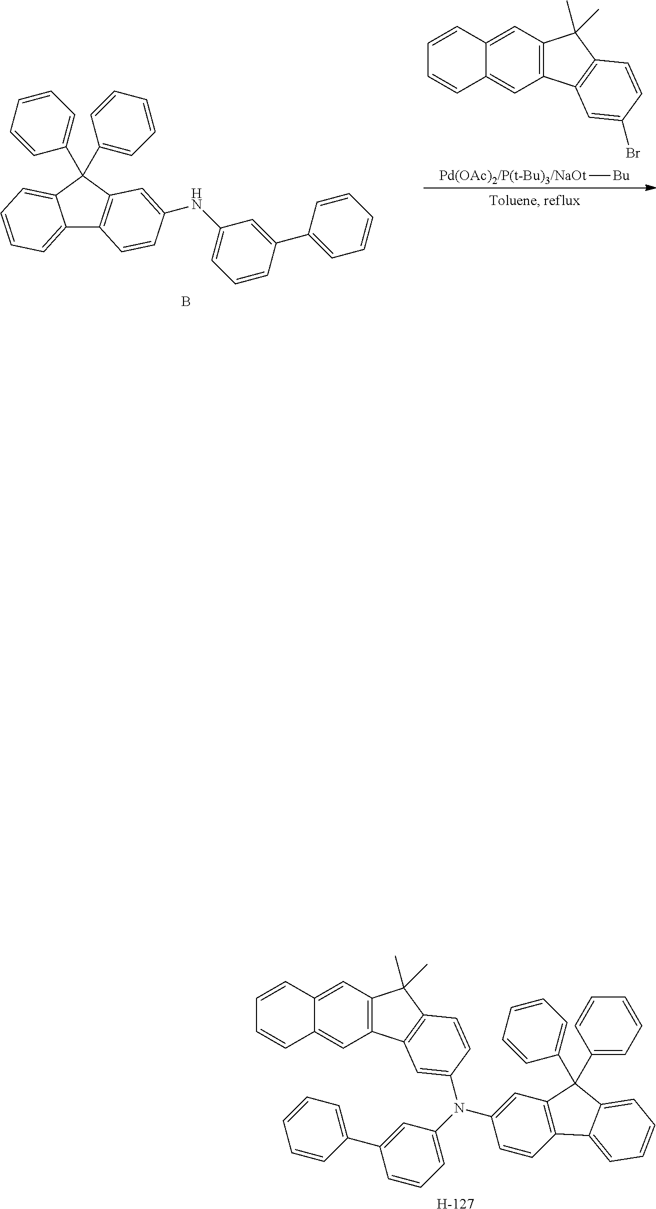

Compound B (9.9 g, 20.42 mmol), 3-bromo-11,11-dimethyl-11H-benzo[b]fluorene (6.0 g, 18.56 mmol), Pd(OAc)₂ (0.9 g, 0.93 mmol), P(t-Bu)₃ (0.9 mL, 1.86 mmol), NaOt-Bu (2.7 g, 27.84 mmol), and 93 mL of toluene were added to the reaction vessel and stirred under reflux at 140° C. for 1 hour. When the reaction was completed, the reaction mixture was cooled to room temperature, and the solid was filtered then washed with ethyl acetate. Next, the filtrate was distilled under reduced pressure and purified by column chromatography to obtain Compound H-127 (2.6 g, yield: 19%).

| | MW | M.P. |
|---|---|---|
| H-127 | 727.93 | 202° C. |

### EXAMPLE 3

### Synthesis of Compound H-204

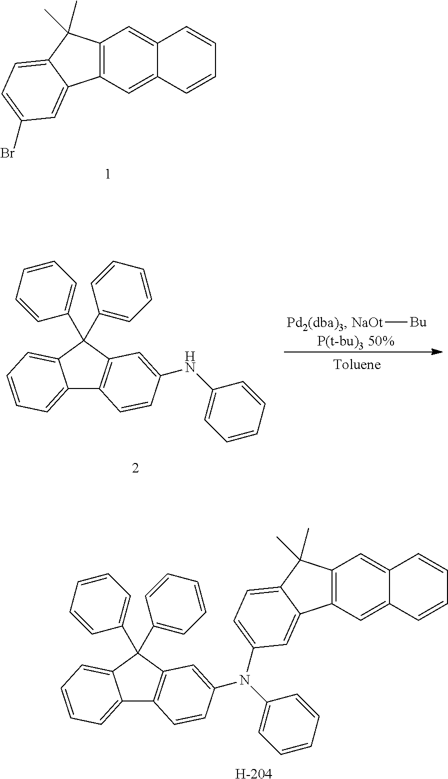

Compound 1 (5 g, 15.46 mmol), Compound 2 (6.3 g, 15.46 mmol), Pd₂(dba)₃ (0.7 g, 0.773 mmol), NaOt-Bu (2.2 g, 23.20 mmol), P(t-Bu)₃ (50%) (0.8 mL, 1.546 mmol), and 80 mL of toluene were added to the reaction vessel and stirred under reflux at 130° C. for 1 hour. When the reaction was completed, the mixture was cooled to room temperature and filtered through Celite. After distillation under reduced pressure, it was separated by column chromatography to obtain Compound H-204 (7.1 g, yield: 71%).

| | MW | M.P. |
|---|---|---|
| H-204 | 651.8 | 236.3° C. |

### EXAMPLE 4

### Synthesis of Compound E-28

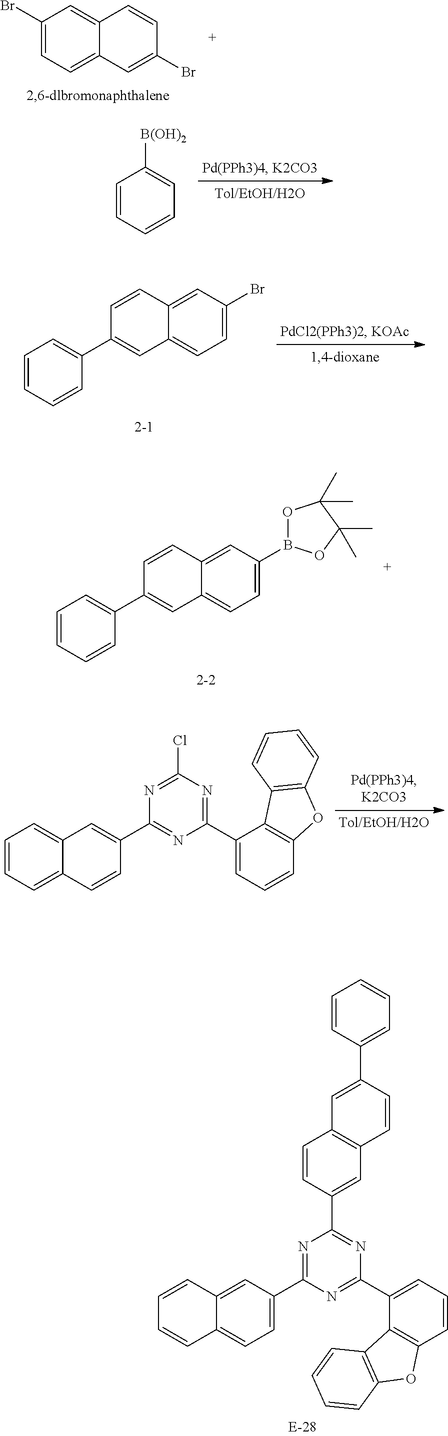 [SMILES: Brc1ccc2cc(Br)ccc2c1 ; OB(O)c1ccccc1 ; Brc1ccc2cc(-c3ccccc3)ccc2c1 ; CC1(C)OB(c2ccc3cc(-c4ccccc4)ccc3c2)OC1(C)C ; Clc1nc(-c2ccc3ccccc3c2)nc(-c2cccc3oc4ccccc4c23)n1 ; c1ccc(-c2ccc3cc(-c4nc(-c5ccc6ccccc6c5)nc(-c5cccc6oc7ccccc7c56)n4)ccc3c2)cc1]

### 1) Synthesis of Compound 2-1

Compound 2,6-dibromonaphthalene (20 g, 70 mmol), phenylboronic acid (9 g, 73.4 mmol), K₂CO₃ (24 g, 175 mmol), tetrakis(triphenylphosphine) palladium(0) (Pd(PPh₃)₄) (4 g, 3.5 mmol), 350 mL of toluene (Tol), 170 mL of H₂O, and 170 mL of ethanol (EtOH) were added to a flask and dissolved, and then refluxed at 130° C. for 1 hour. When the reaction was completed, the organic layer was extracted with ethyl acetate and the residual moisture was removed using magnesium sulfate, followed by drying then separation using column chromatography to obtain Compound 2-1 (13 g, yield: 67%).

### 2) Synthesis of Compound 2-2

Compound 2-1 (13 g, 45.9 mmol), (4,4,4′,4′,5,5,5′,5′-octamethyl-2,2′-bi(1,3,2-dioxaborolane) (17.5 g, 68.8 mmol), KOAc (11.3 g, 114.75 mmol), PdCl₂(PPh₃)₂ (3.2 g, 4.59 mmol), and 230 mL of 1,4-dioxane were added to a flask and dissolved, and then refluxed at 150° C. for 2 hours. When the reaction was completed, the organic layer was extracted with ethyl acetate and the residual moisture was removed using magnesium sulfate, followed by drying then separation using column chromatography to obtain Compound 2-2 (9 g, yield: 59.3%).

### 3) Synthesis of Compound E-28

Compound 2-2 (6.4 g 19.16 mmol), 2-chloro-4-(dibenzo[b,d]furan-1-yl)-6-(naphthalen-2-yl)-1,3,5-triazine (6.5 g, 15.96 mmol), K₂CO₃ (5.5 g, 39.9 mmol), Pd(PPh₃)₄ (922 mg, 0.798 mmol), 80 mL of toluene , 40 mL of EtOH, and 40 mL of H₂O were added to a flask and dissolved, and then refluxed at 130° C. for 2 hours. When the reaction was completed, the organic layer was extracted with ethyl acetate and the residual moisture was removed using magnesium sulfate, followed by drying, then separation using column chromatography to obtain Compound E-28 (4.9 g, yield: 53.3%).

| | MW | M.P. |
|---|---|---|
| E-28 | 575.20 | 242.5° C. |

### EXAMPLE 5

### Synthesis of Compound E-26

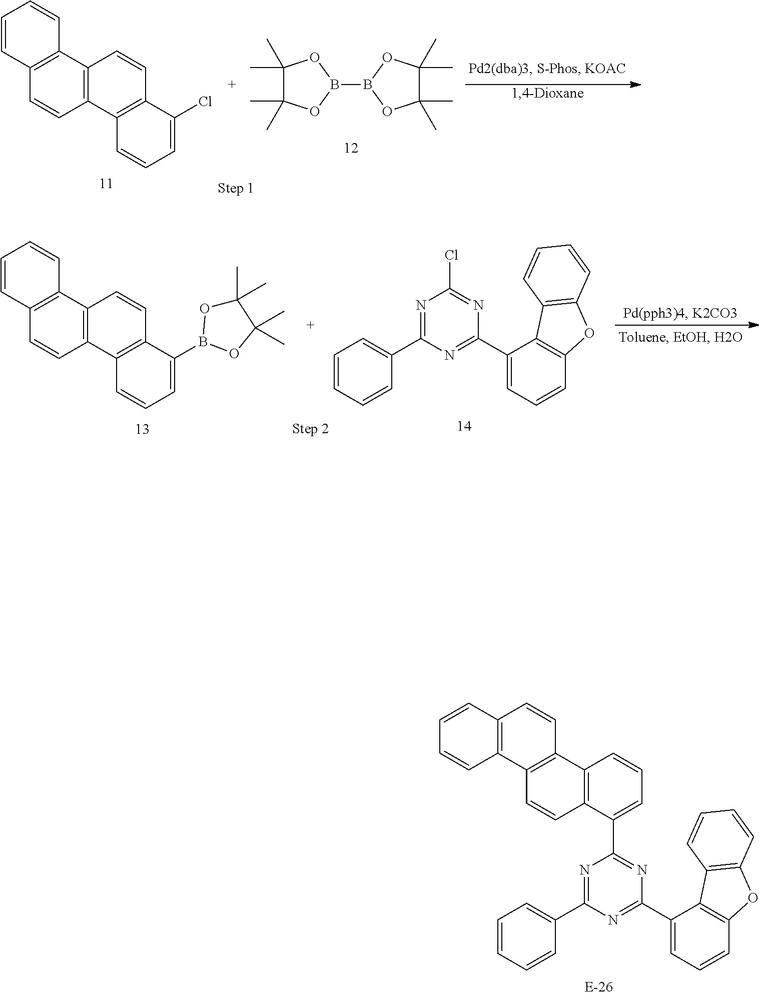

### 1) Synthesis of Compound 13

Compound 11 (10.0 g, 38.07 mmol), Compound 12 (19.3 g, 76.13 mmol), Pd₂(dba)₃ (1.7g, 1.90 mmol), S-Phos (1.6 g, 3.81 mmol), KOAC (9.3 g, 95.17 mmol) were dissolved in 190 mL of 1,4-dioxane, and then stirred under reflux at 150° C. for 3 hours. When the reaction was completed, the mixture was cooled to room temperature, followed by filtration through a Celite filter then a silica filter, and then solidified to obtain Compound 13 (9.8 g, yield: 72.70%).

### 2) Synthesis of Compound E-26

Compound 13 (8.8 g, 24.84 mmol), Compound 14 (11.6 g, 32.29 mmol), Pd(pph₃)₄ (1.4g, 1.24 mmol), and K₂CO₃ (6.9 g, 49.68 mmol) were dissolved in 125 mL of toluene, 31 mL of EtOH, and 31 mL of H₂O, and stirred under reflux at 130° C. for 6 hours. When the reaction was completed, the mixture was cooled to room temperature, followed by filtration through a Celite filter and then a silica filter to make a solid, and then recrystallized to obtain Compound E-26 (10.4 g, yield: 76.47%).

| | MW | M.P |
|---|---|---|
| E-26 | 549.63 | 281° C. |

Hereinafter, the preparation method for an organic electroluminescent device comprising the plurality of host materials according to the present disclosure, and the device property thereof, will be explained in order to understand the present disclosure in detail.

### DEVICE EXAMPLES 1 TO 4

### Preparation of OLEDs Comprising a Plurality of Host Materials According to the Present Disclosure

OLEDs according to the present disclosure were prepared. First, a transparent electrode indium tin oxide (ITO) thin film (10 Q/sq) on a glass substrate for an OLED (GEOMATEC CO., LTD., Japan) was subjected to an ultrasonic washing with acetone and isopropyl alcohol, sequentially; thereafter, it was stored in isopropyl alcohol and used. Next, the ITO substrate was mounted on a substrate holder of a vacuum vapor deposition apparatus. Then, Compound H-204 was introduced into a cell of the vacuum vapor deposition apparatus, while Compound HT-1 was introduced into another cell. The two materials were evaporated at different rates and Compound H-204 was deposited in a doping amount of 3 wt % based on the total amount of Compounds H-204 and HT-1 to form a hole injection layer having a thickness of 10 nm. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host compound and the second host compound described in the following Tables 1 to 3 were introduced into two cells of the vacuum vapor deposition apparatus as hosts, respectively, and Compound D-39 was introduced into another cell as a dopant. The two host materials were evaporated at a rate of 1:1, and the dopant material was evaporated at a different rate, simultaneously, and the dopant was deposited in a doping amount of 3 wt % based on the total amount of the hosts and dopant to form a light-emitting layer having a thickness of 40 nm on the second hole transport layer. Next, compounds ETL-1 and EIL-1 as electron transport materials were deposited at a weight ratio of 50:50 to form an electron transport layer having a thickness of 35 nm on the light-emitting layer. After depositing Compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### COMPARATIVE EXAMPLES 1 TO 6

### Preparation of OLEDs Comprising Conventional Host Combinations Not According to the Present Disclosure

OLEDs were manufactured in the same manner as in Device Examples 1 to 4, except that the host compound from the following Tables 1 to 3 were used as the second host compound for the light-emitting layer.

The driving voltage, the luminous efficiency and the luminous color at a luminance of 5,000 nits as well as the time taken for luminance to decrease from 100% to 95% at a luminance of 10,000 nits (lifespan: T95) for the OLEDs of Device Examples 1 to 4 and Comparative Examples 1 to 6 produced as described above, are measured, and the results thereof are shown in the following Tables 1 to 3.

TABLE 1

| | First | Second | Driving | Luminous | | |
|---|---|---|---|---|---|---|
| | Host | Host | Voltage | Efficiency | Luminous | Lifespan |
| | Compound | Compound | (V) | (cd/A) | Color | T95(hr) |
| Device | H-204 | E-28 | 3.8 | 29.2 | Red | 120 |
| Example 1 | | | | | | |
| Device | H-204 | E-26 | 3.9 | 32.9 | Red | 166 |
| Example 2 | | | | | | |
| Comparative | H-204 | Ref-1 | 3.6 | 31.3 | Red | 63 |
| Example 1 | | | | | | |
| Comparative | H-204 | Ref-2 | 4.1 | 29.9 | Red | 76 |
| Example 2 | | | | | | |

TABLE 2

| | First | Second | Driving | Luminous | | |
|---|---|---|---|---|---|---|
| | Host | Host | Voltage | Efficiency | Luminous | Lifespan |
| | Compound | Compound | (V) | (cd/A) | Color | T95(hr) |
| Device | H-94 | E-26 | 3.8 | 32.2 | Red | 148 |
| Example 3 | | | | | | |
| Comparative | H-94 | Ref-1 | 3.5 | 31.1 | Red | 73 |
| Example 3 | | | | | | |
| Comparative | H-94 | Ref-2 | 3.9 | 31.3 | Red | 99 |
| Example 4 | | | | | | |

TABLE 3

| | First | Second | Driving | Luminous | | |
|---|---|---|---|---|---|---|
| | Host | Host | Voltage | Efficiency | Luminous | Lifespan |
| | Compound | Compound | (V) | (cd/A) | Color | T95(hr) |
| Device | H-127 | E-26 | 3.9 | 31.3 | Red | 96 |
| Example 4 | | | | | | |
| Comparative | H-127 | Ref-1 | 3.7 | 30.5 | Red | 33 |
| Example 5 | | | | | | |
| Comparative | H-127 | Ref-2 | 4.2 | 29.1 | Red | 47 |
| Example 6 | | | | | | |

From Tables 1 to 3 above, it can be seen that the organic electroluminescent device (Device Examples 1 to 4) comprising a specific combination of compounds according to present disclosure as host materials exhibits low driving voltage and/or high luminous efficiency, and in particular, significantly improved lifespan characteristics compared to the organic electroluminescent device (Comparative Examples 1 to 6) comprsing a host combination not according to the present disclosure.

The compounds used in Device Examples and Comparative Examples above are specifically shown in the following Table 4.

TABLE 4

| | |
|---|---|
| Hole Injection Layer/Hole Transport Layer/ Electron blocking layer | 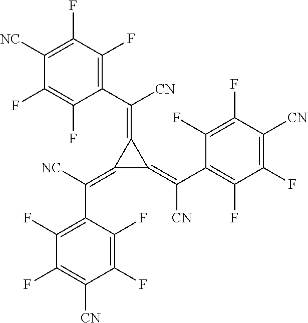 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] HI-1 |
| | 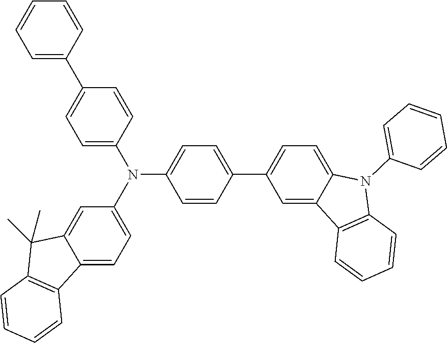 HT-1 |
| | 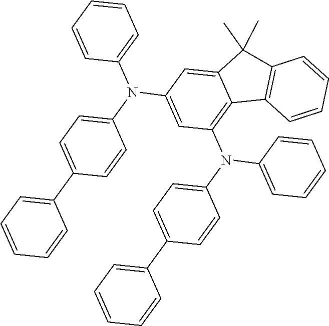 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] HT-2 |
| Light-Emitting Layer | 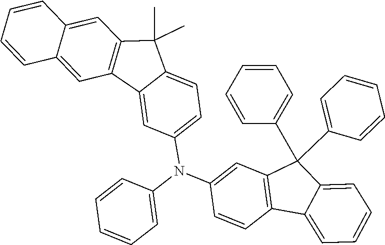 [SMILES: CC1(C)c2ccc(N(c3ccccc3)c3ccc4c(c3)C(c3ccccc3)(c3ccccc3)c3ccccc3-4)cc2-c2cc3ccccc3cc21] H-204 |
| | 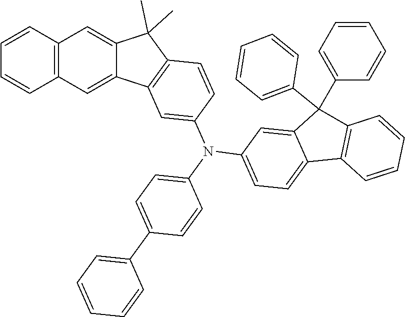 [SMILES: CC1(C)c2ccc(N(c3ccc(-c4ccccc4)cc3)c3ccc4c(c3)C(c3ccccc3)(c3ccccc3)c3ccccc3-4)cc2-c2cc3ccccc3cc21] H-94 |
| | 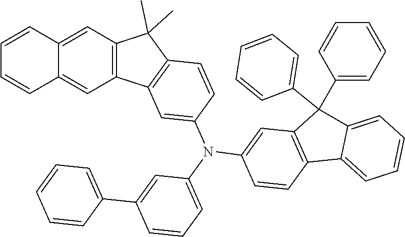 [SMILES: CC1(C)c2ccc(N(c3cccc(-c4ccccc4)c3)c3ccc4c(c3)C(c3ccccc3)(c3ccccc3)c3ccccc3-4)cc2-c2cc3ccccc3cc21] H-127 |
| | 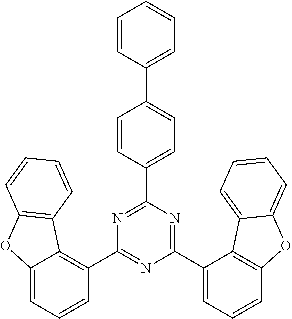 [SMILES: c1ccc(-c2ccc(-c3nc(-c4cccc5oc6ccccc6c45)nc(-c4cccc5oc6ccccc6c45)n3)cc2)cc1] Ref-1 |
| | 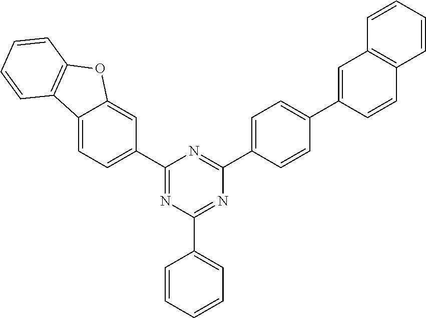 [SMILES: c1ccc(-c2nc(-c3ccc(-c4ccc5ccccc5c4)cc3)nc(-c3ccc4c(c3)oc3ccccc34)n2)cc1] Ref-2 |
| | 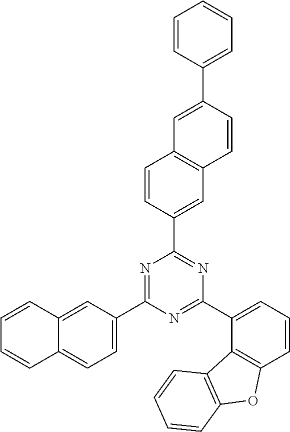 [SMILES: c1ccc(-c2ccc3cc(-c4nc(-c5ccc6ccccc6c5)nc(-c5cccc6oc7ccccc7c56)n4)ccc3c2)cc1] E-28 |
| | 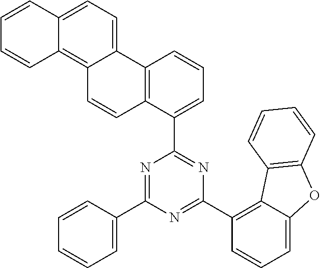 [SMILES: c1ccc(-c2nc(-c3cccc4c3ccc3c5ccccc5ccc43)nc(-c3cccc4oc5ccccc5c34)n2)cc1] E-26 |
| | 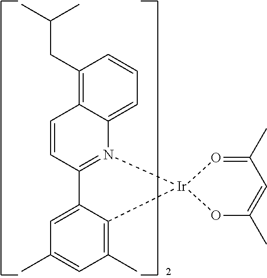 D-39 |
| Electron Transport Layer/ Electron Injection Layer | 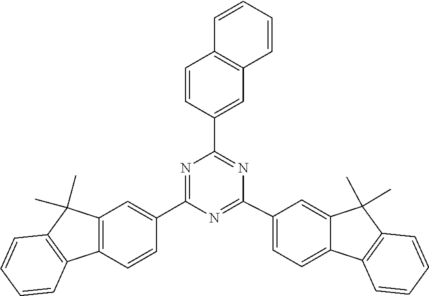 [SMILES: CC1(C)c2ccccc2-c2ccc(-c3nc(-c4ccc5c(c4)C(C)(C)c4ccccc4-5)nc(-c4ccc5ccccc5c4)n3)cc21] ETL-1 |
| | 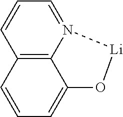 EIL-1 |

1. A plurality of host materials comprising at least one first host compound represented by the following Formula 1 and at least one second host compound represented by the following Formula 2. 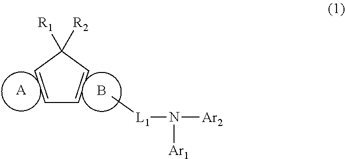
wherein,
ring A and ring B each independently represent, a benzene ring or a naphthalene ring; provided that at least one of ring A and ring B is a naphthalene ring;
R₁ and R₂ each independently represent, hydrogen, deuterium, halogen, cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono-or di-(C1-C30)alkylamino, a substituted or unsubstituted mono-or di-(C6-C30)arylamino, or a substituted or unsubstituted (C1-C30)alkyl(C6-C30)arylamino; or may be linked to the adjacent substituents to form a ring(s);
L₁ represents, a single bond, a substituted or unsubstituted (C6-C30)arylene, or a substituted or unsubstituted (3-to 30-membered)heteroarylene; and
Ar₁ and Ar₂ each independently represent, a substituted or unsubstituted (C6-C30)aryl or a substituted or unsubstituted (3-to 30-membered)heteroaryl; 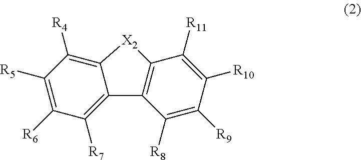
wherein,
X₂ represents, —O—, —S— or —Se—;
R₄ to R₆ and R₈ to R₁₁ each independently represent, hydrogen, deuterium, or a substituted or unsubstituted (C6-C30)aryl; provided that the (C6-C30)aryl does not contain a substituted or unsubstituted fluorenyl; and
R₇ represents, the following Formula 2-a; 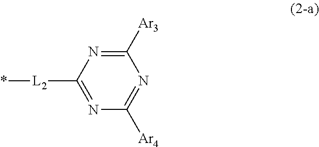
wherein,
L₂ represents, a single bond, a substituted or unsubstituted (C6-C30)arylene, or a substituted or unsubstituted (3-30-membered)heteroarylene; and
Ar₃ and Ar₄ each independently represent, a substituted or unsubstituted (C6-C30)aryl, but do not contain a substituted or unsubstituted fluorenyl.

2. The plurality of host materials according to claim 1, wherein the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted mono-or di-alkylamino, the substituted mono-or di-arylamino, and the substituted alkylarylamino each independently are substituted with at least one selected from the group consisting of deuterium; halogen; cyano; carboxyl; nitro; hydroxy; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3-to 7-membered)heterocycloalkyl; (C6-C30)aryloxy; (C6-C30)arylthio; (3-to 30-membered)heteroaryl unsubstituted or substituted with at least one (C6-C30)aryl; (C6-C30)aryl unsubstituted or substituted with at least one of deuterium, halogen, cyano, (C1-C30)alkyl, (C6-C30)aryl, (3-to 30-membered)heteroaryl and tri(C6-C30)arylsilyl; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono-or di-(C1-C30)alkylamino; mono-or di-(C2-C30)alkenylamino; mono-or di-(C6-C30)arylamino; mono-or di-(3-30-membered)heteroarylamino; (C1-C30)alkyl(C2-C30)alkenylamino; (C1-C30)alkyl(C6-C30)arylamino; (C1-C30)alkyl(3-to 30-membered)heteroarylamino; (C2-C30)alkenyl(C6-C30)arylamino; (C2-C30)alkenyl(3-to 30-membered)heteroarylamino; (C6-C30)aryl(3-30-membered) heteroarylamino; (C1-C30)alkylcarbonyl; (C1-C30)alkoxycarbonyl; (C6-C30)arylcarbonyl; di(C6-C30)arylboronyl; (C6-C30)arylphosphine; di(C1-C30)alkylboronyl; (C1-C30)alkyl(C6-C30)arylboronyl; (C6-C30)ar(C1-C30)alkyl; and (C1-C30)alkyl(C6-C30)aryl.

3. The plurality of host materials according to claim 1, wherein the Formula 1 is represented by any one of the following Formulas 1-1 to 1-6: 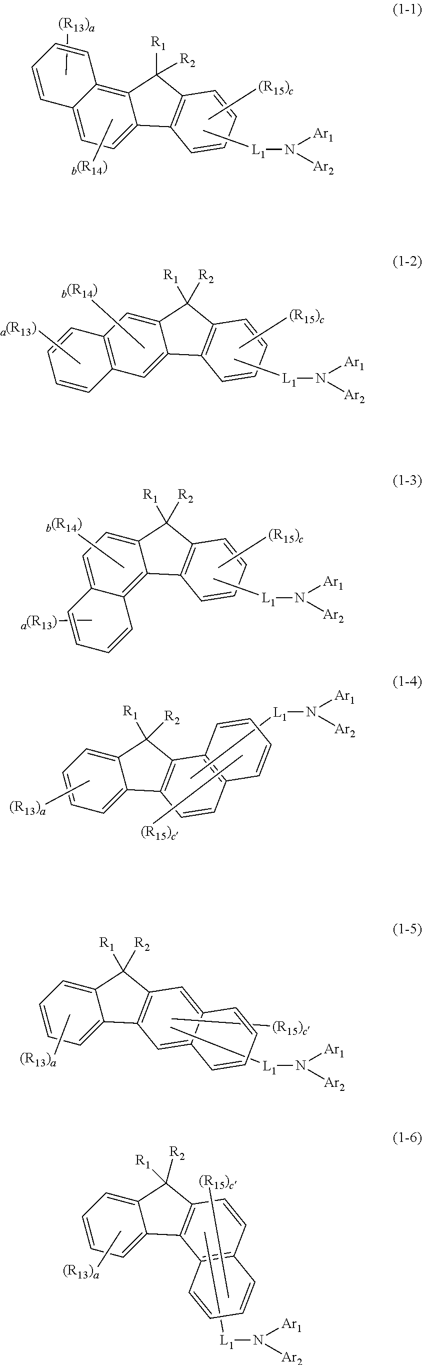
wherein,
R₁, R₂, L₁, Ar₁, and Ar₂ are as defined in claim 1;
R₁₃ to R₁₅ are as defined as R₁ in claim 1; and
a represents an integer of 1 to 4; b represents an integer of 1 or 2; c represents an integer of 1 to 3; and c′ represents an integer of 1 to 5; when a to c and c′ are an integer of 2 or more, each of R₁₃ to R₁₅ may be the same or different.

4. The plurality of host materials according to claim 1, wherein Ar₃ and Ar₄ in Formula 2 each independently are selected from phenyl unsubstituted or substituted with deuterium, naphthyl unsubstituted or substituted with deuterium, biphenyl unsubstituted or substituted with deuterium, terphenyl unsubstituted or substituted with deuterium, phenanthrenyl unsubstituted or substituted with deuterium, chrysenyl unsubstituted or substituted with deuterium, or a combination thereof.

5. The plurality of host materials according to claim 1, wherein at least one of Ar₃ and Ar₄ in Formula 2 is naphthyl unsubstituted or substituted with deuterium or phenyl, a substituted or unsubstituted phenanthrenyl, or a substituted or unsubstituted chrysenyl.

6. The plurality of host materials according to claim 1, wherein the compound represented by the Formula 1 is selected from the following compounds:

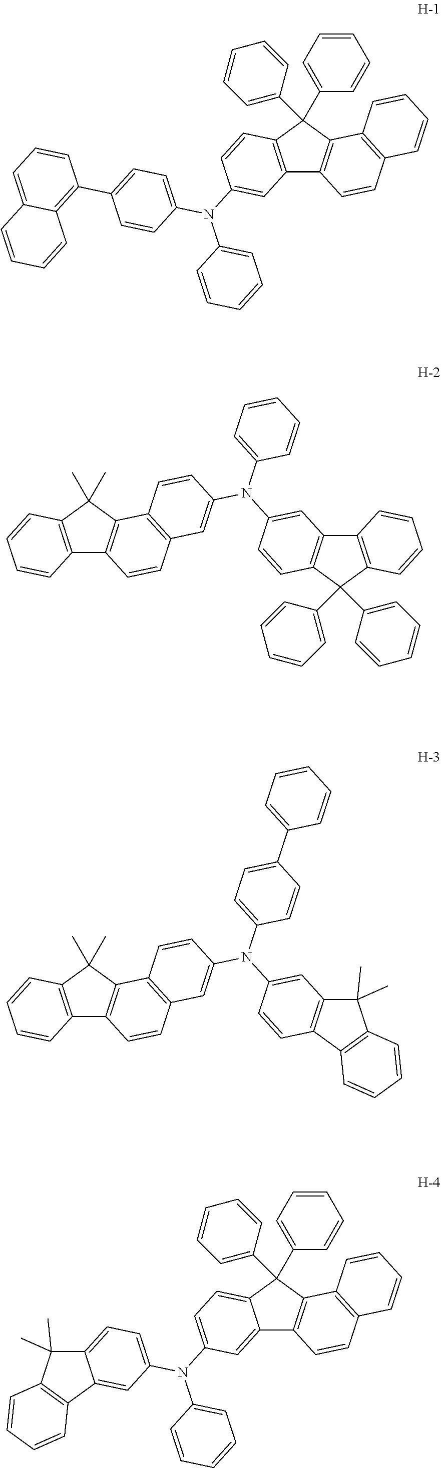 [SMILES: c1ccc(N(c2ccc(-c3cccc4ccccc34)cc2)c2ccc3c(c2)-c2ccc4ccccc4c2C3(c2ccccc2)c2ccccc2)cc1 ; CC1(C)c2ccccc2-c2ccc3cc(N(c4ccccc4)c4ccc5c(c4)-c4ccccc4C5(c4ccccc4)c4ccccc4)ccc3c21 ; CC1(C)c2ccccc2-c2ccc(N(c3ccc(-c4ccccc4)cc3)c3ccc4c5c(ccc4c3)-c3ccccc3C5(C)C)cc21 ; CC1(C)c2ccccc2-c2cc(N(c3ccccc3)c3ccc4c(c3)-c3ccc5ccccc5c3C4(c3ccccc3)c3ccccc3)ccc21]

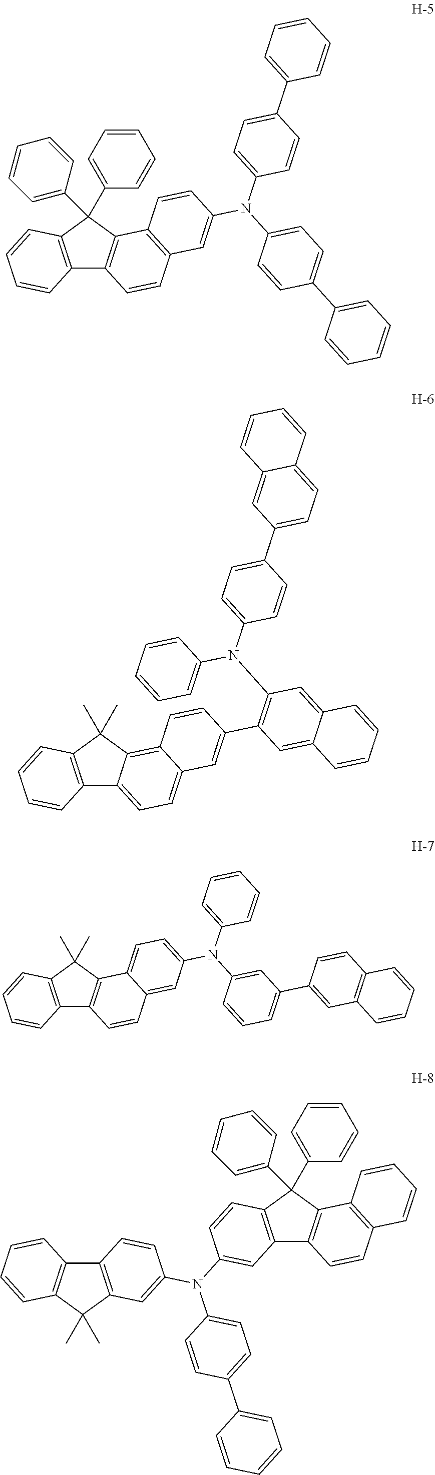 [SMILES: c1ccc(-c2ccc(N(c3ccc(-c4ccccc4)cc3)c3ccc4c5c(ccc4c3)-c3ccccc3C5(c3ccccc3)c3ccccc3)cc2)cc1 ; CC1(C)c2ccccc2-c2ccc3cc(-c4cc5ccccc5cc4N(c4ccccc4)c4ccc(-c5ccc6ccccc6c5)cc4)ccc3c21 ; CC1(C)c2ccccc2-c2ccc3cc(N(c4ccccc4)c4cccc(-c5ccc6ccccc6c5)c4)ccc3c21 ; CC1(C)c2ccccc2-c2ccc(N(c3ccc(-c4ccccc4)cc3)c3ccc4c(c3)-c3ccc5ccccc5c3C4(c3ccccc3)c3ccccc3)cc21]

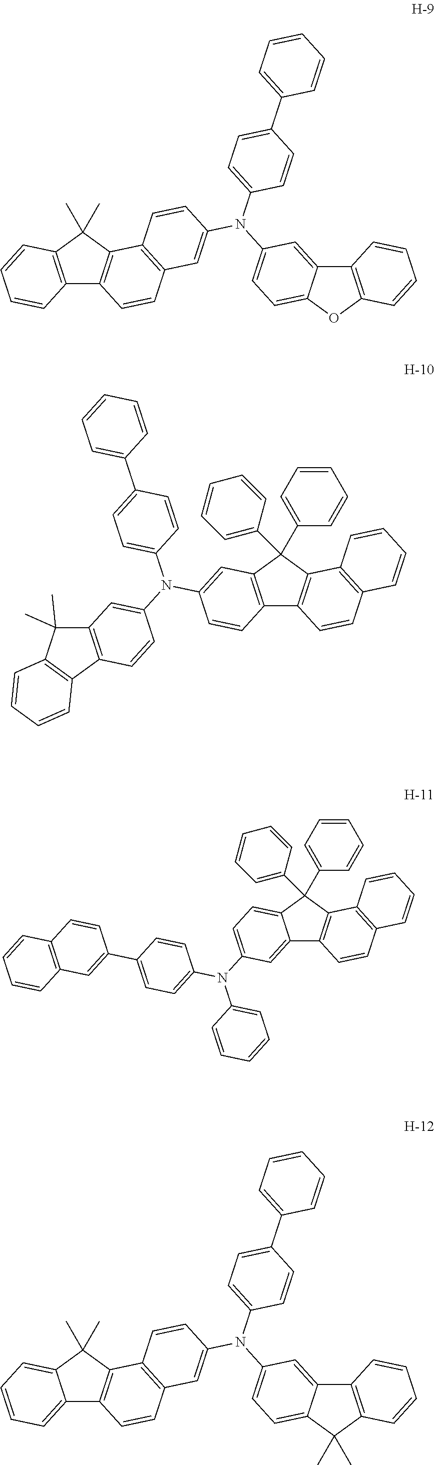 [SMILES: CC1(C)c2ccccc2-c2ccc3cc(N(c4ccc(-c5ccccc5)cc4)c4ccc5oc6ccccc6c5c4)ccc3c21 ; CC1(C)c2ccccc2-c2ccc(N(c3ccc(-c4ccccc4)cc3)c3ccc4c(c3)C(c3ccccc3)(c3ccccc3)c3c-4ccc4ccccc34)cc21 ; c1ccc(N(c2ccc(-c3ccc4ccccc4c3)cc2)c2ccc3c(c2)-c2ccc4ccccc4c2C3(c2ccccc2)c2ccccc2)cc1 ; CC1(C)c2ccccc2-c2cc(N(c3ccc(-c4ccccc4)cc3)c3ccc4c5c(ccc4c3)-c3ccccc3C5(C)C)ccc21]

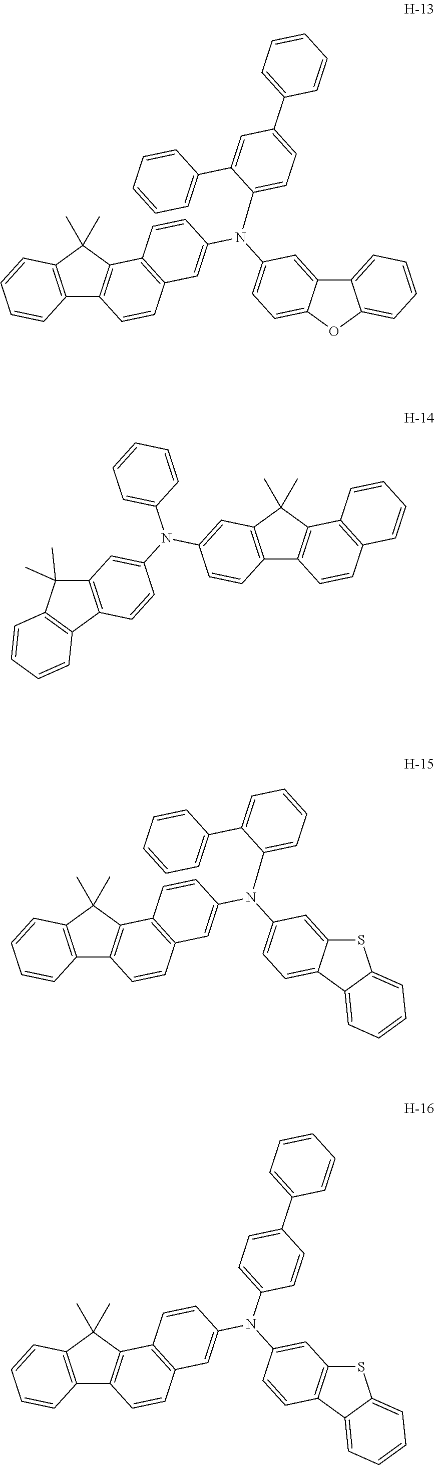 [SMILES: CC1(C)c2ccccc2-c2ccc3cc(N(c4ccc5oc6ccccc6c5c4)c4ccc(-c5ccccc5)cc4-c4ccccc4)ccc3c21 ; CC1(C)c2ccccc2-c2ccc(N(c3ccccc3)c3ccc4c(c3)C(C)(C)c3c-4ccc4ccccc34)cc21 ; CC1(C)c2ccccc2-c2ccc3cc(N(c4ccc5c(c4)sc4ccccc45)c4ccccc4-c4ccccc4)ccc3c21 ; CC1(C)c2ccccc2-c2ccc3cc(N(c4ccc(-c5ccccc5)cc4)c4ccc5c(c4)sc4ccccc45)ccc3c21]

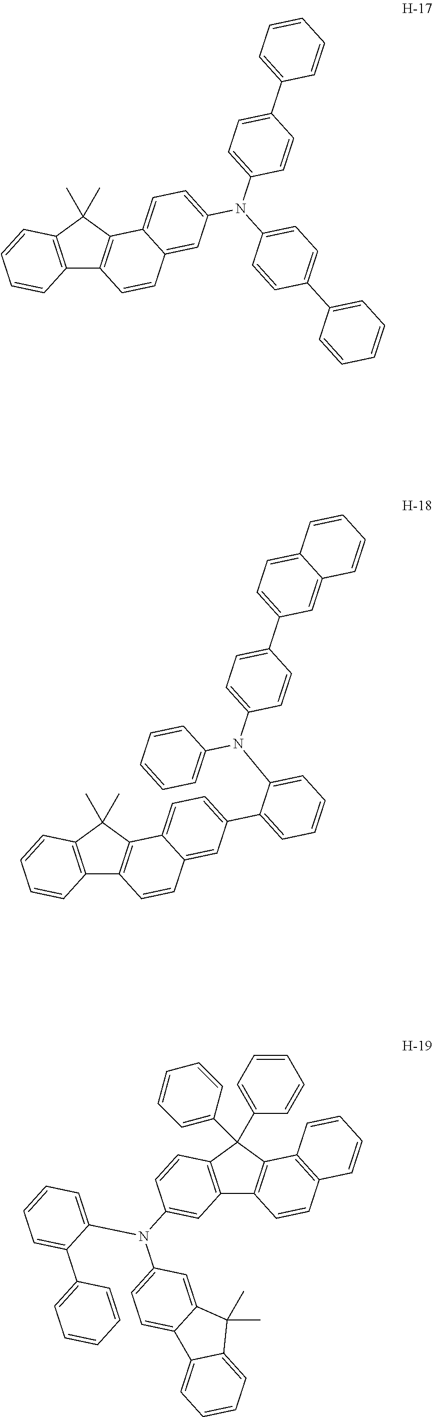

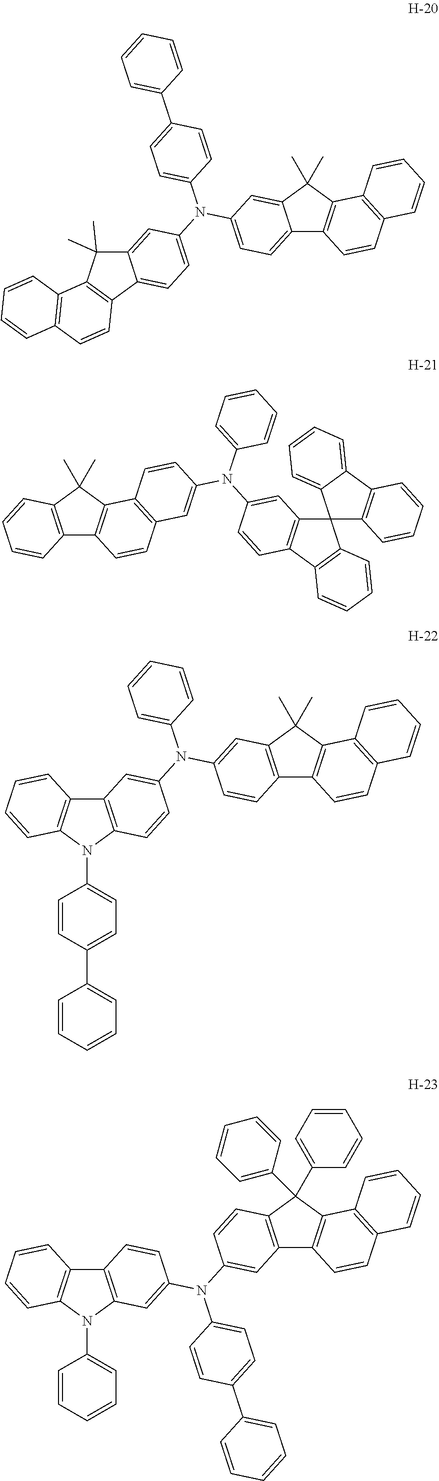 [SMILES: CC1(C)c2cc(N(c3ccc(-c4ccccc4)cc3)c3ccc4c(c3)C(C)(C)c3c-4ccc4ccccc34)ccc2-c2ccc3ccccc3c21 ; CC1(C)c2ccccc2-c2ccc3cc(N(c4ccccc4)c4ccc5c(c4)C4(c6ccccc6-c6ccccc64)c4ccccc4-5)ccc3c21 ; CC1(C)c2cc(N(c3ccccc3)c3ccc4c(c3)c3ccccc3n4-c3ccc(-c4ccccc4)cc3)ccc2-c2ccc3ccccc3c21 ; c1ccc(-c2ccc(N(c3ccc4c(c3)-c3ccc5ccccc5c3C4(c3ccccc3)c3ccccc3)c3ccc4c5ccccc5n(-c5ccccc5)c4c3)cc2)cc1]

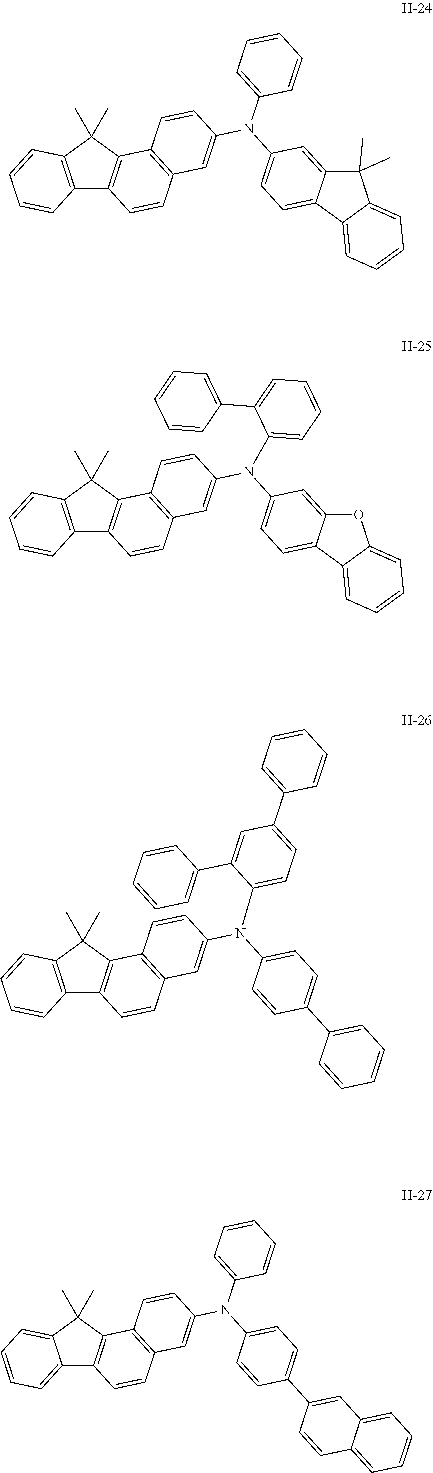 [SMILES: CC1(C)c2ccccc2-c2ccc(N(c3ccccc3)c3ccc4c5c(ccc4c3)-c3ccccc3C5(C)C)cc21 ; CC1(C)c2ccccc2-c2ccc3cc(N(c4ccc5c(c4)oc4ccccc45)c4ccccc4-c4ccccc4)ccc3c21 ; CC1(C)c2ccccc2-c2ccc3cc(N(c4ccc(-c5ccccc5)cc4)c4ccc(-c5ccccc5)cc4-c4ccccc4)ccc3c21 ; CC1(C)c2ccccc2-c2ccc3cc(N(c4ccccc4)c4ccc(-c5ccc6ccccc6c5)cc4)ccc3c21]

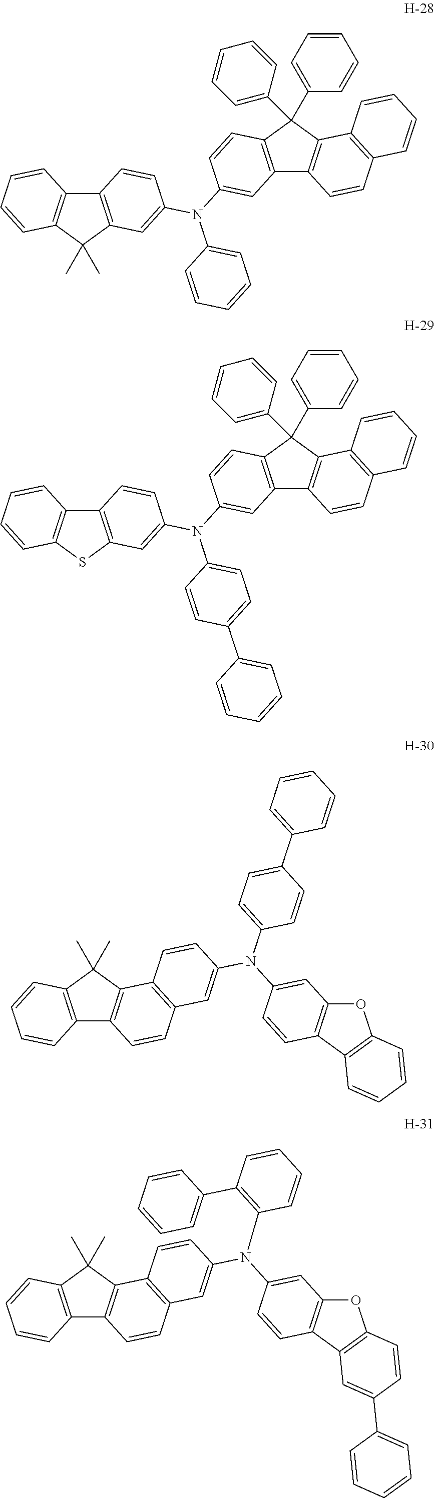 [SMILES: CC1(C)c2ccccc2-c2ccc(N(c3ccccc3)c3ccc4c(c3)-c3ccc5ccccc5c3C4(c3ccccc3)c3ccccc3)cc21 ; c1ccc(-c2ccc(N(c3ccc4c(c3)-c3ccc5ccccc5c3C4(c3ccccc3)c3ccccc3)c3ccc4c(c3)sc3ccccc34)cc2)cc1 ; CC1(C)c2ccccc2-c2ccc3cc(N(c4ccc(-c5ccccc5)cc4)c4ccc5c(c4)oc4ccccc45)ccc3c21 ; CC1(C)c2ccccc2-c2ccc3cc(N(c4ccc5c(c4)oc4ccc(-c6ccccc6)cc45)c4ccccc4-c4ccccc4)ccc3c21]

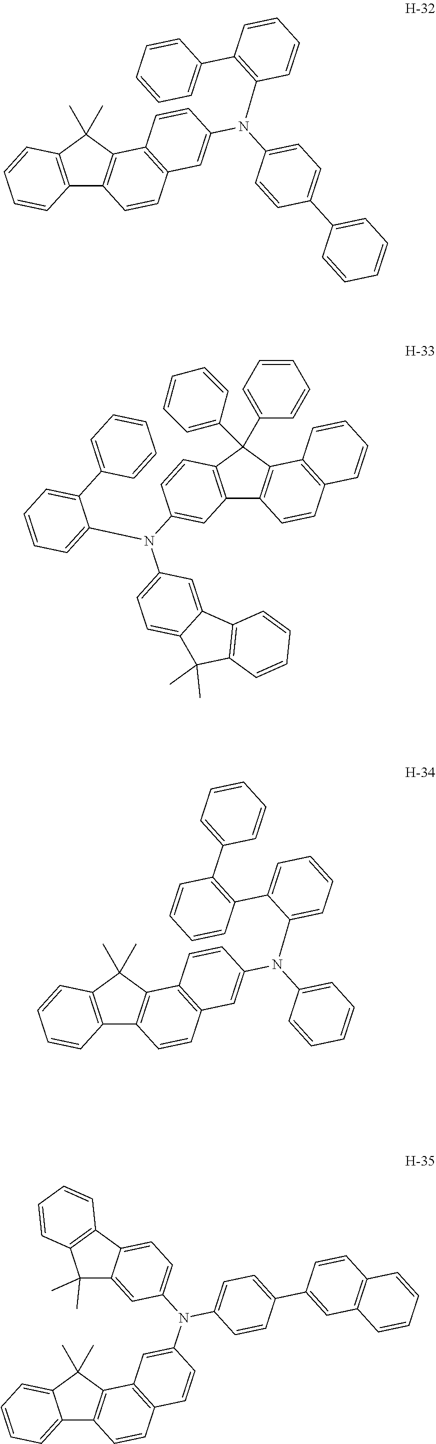 [SMILES: CC1(C)c2ccccc2-c2ccc3cc(N(c4ccc(-c5ccccc5)cc4)c4ccccc4-c4ccccc4)ccc3c21 ; CC1(C)c2ccccc2-c2cc(N(c3ccc4c(c3)-c3ccc5ccccc5c3C4(c3ccccc3)c3ccccc3)c3ccccc3-c3ccccc3)ccc21 ; CC1(C)c2ccccc2-c2ccc3cc(N(c4ccccc4)c4ccccc4-c4ccccc4-c4ccccc4)ccc3c21 ; CC1(C)c2ccccc2-c2ccc(N(c3ccc(-c4ccc5ccccc5c4)cc3)c3ccc4ccc5c(c4c3)C(C)(C)c3ccccc3-5)cc21]

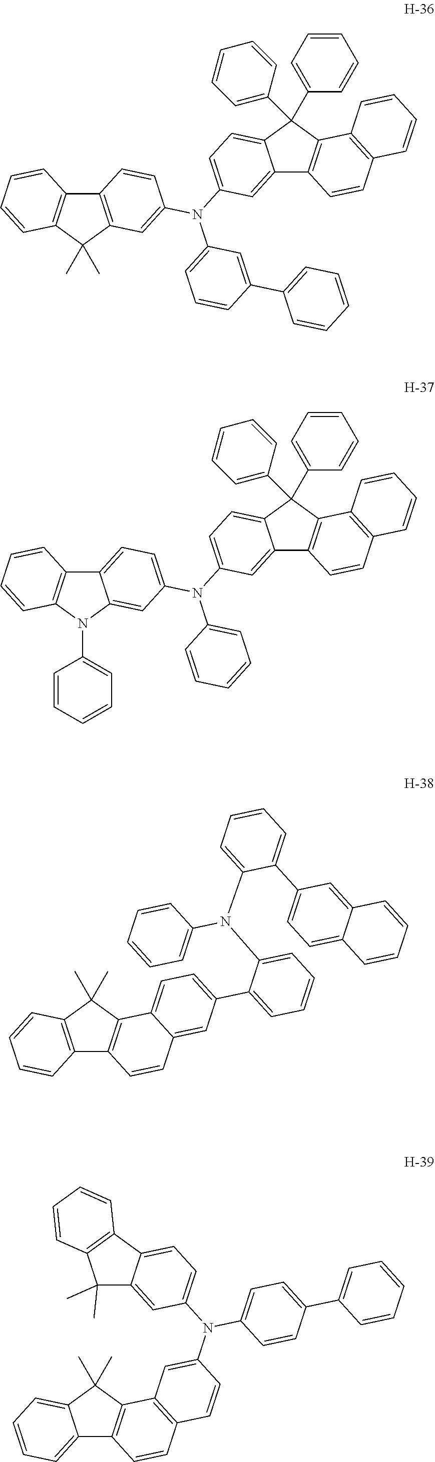 [SMILES: CC1(C)c2ccccc2-c2ccc(N(c3cccc(-c4ccccc4)c3)c3ccc4c(c3)-c3ccc5ccccc5c3C4(c3ccccc3)c3ccccc3)cc21 ; c1ccc(N(c2ccc3c(c2)-c2ccc4ccccc4c2C3(c2ccccc2)c2ccccc2)c2ccc3c4ccccc4n(-c4ccccc4)c3c2)cc1 ; CC1(C)c2ccccc2-c2ccc3cc(-c4ccccc4N(c4ccccc4)c4ccccc4-c4ccc5ccccc5c4)ccc3c21 ; CC1(C)c2ccccc2-c2ccc(N(c3ccc(-c4ccccc4)cc3)c3ccc4ccc5c(c4c3)C(C)(C)c3ccccc3-5)cc21]

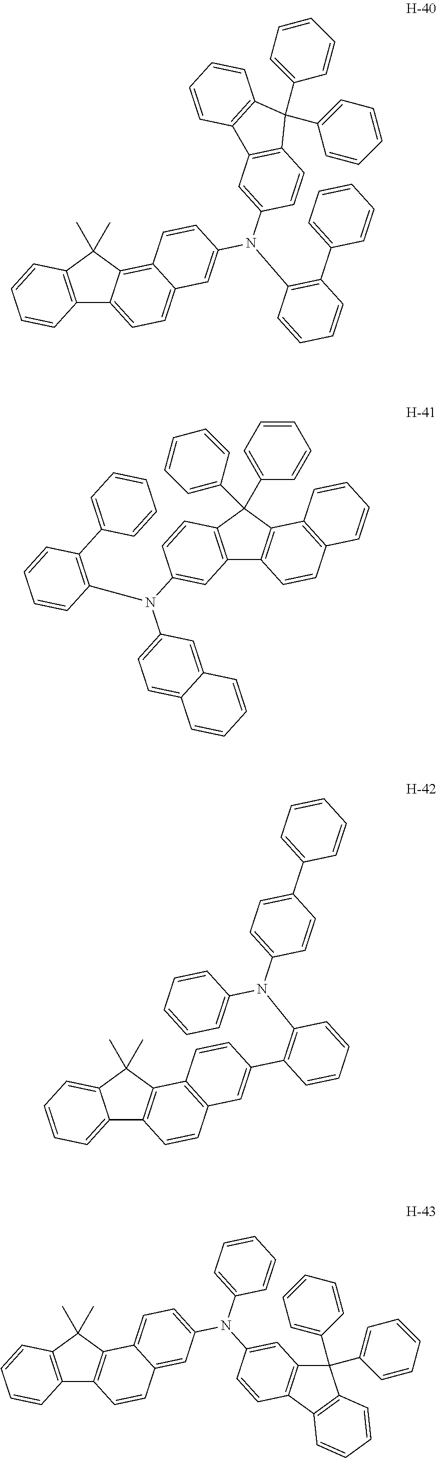 [SMILES: CC1(C)c2ccccc2-c2ccc3cc(N(c4ccc5c(c4)-c4ccccc4C5(c4ccccc4)c4ccccc4)c4ccccc4-c4ccccc4)ccc3c21 ; c1ccc(-c2ccccc2N(c2ccc3c(c2)-c2ccc4ccccc4c2C3(c2ccccc2)c2ccccc2)c2ccc3ccccc3c2)cc1 ; CC1(C)c2ccccc2-c2ccc3cc(-c4ccccc4N(c4ccccc4)c4ccc(-c5ccccc5)cc4)ccc3c21 ; CC1(C)c2ccccc2-c2ccc3cc(N(c4ccccc4)c4ccc5c(c4)C(c4ccccc4)(c4ccccc4)c4ccccc4-5)ccc3c21]

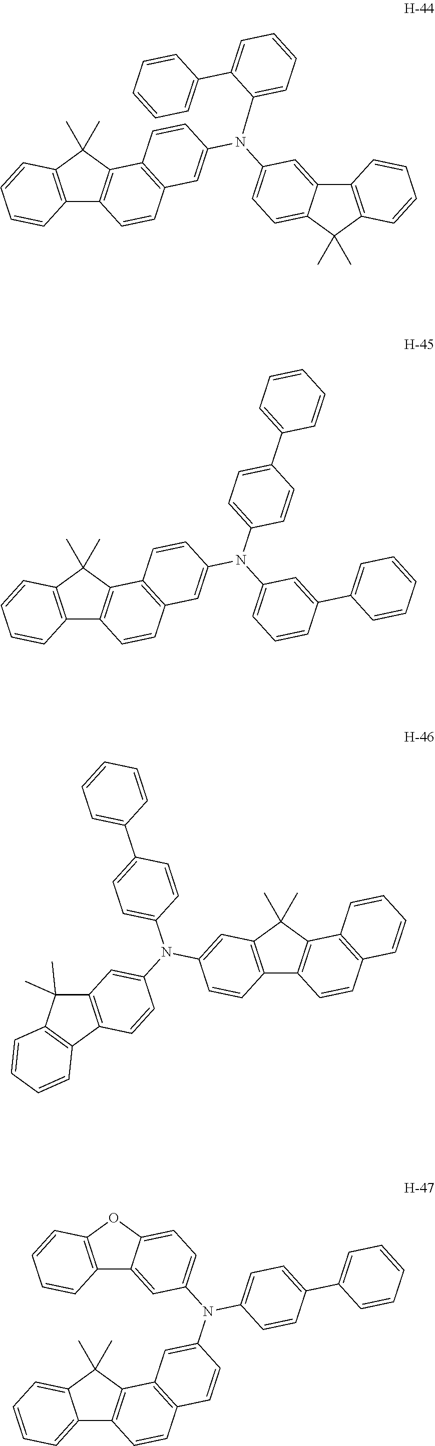 [SMILES: CC1(C)c2ccccc2-c2cc(N(c3ccc4c5c(ccc4c3)-c3ccccc3C5(C)C)c3ccccc3-c3ccccc3)ccc21 ; CC1(C)c2ccccc2-c2ccc3cc(N(c4ccc(-c5ccccc5)cc4)c4cccc(-c5ccccc5)c4)ccc3c21 ; CC1(C)c2ccccc2-c2ccc(N(c3ccc(-c4ccccc4)cc3)c3ccc4c(c3)C(C)(C)c3c-4ccc4ccccc34)cc21 ; CC1(C)c2ccccc2-c2ccc3ccc(N(c4ccc(-c5ccccc5)cc4)c4ccc5oc6ccccc6c5c4)cc3c21]

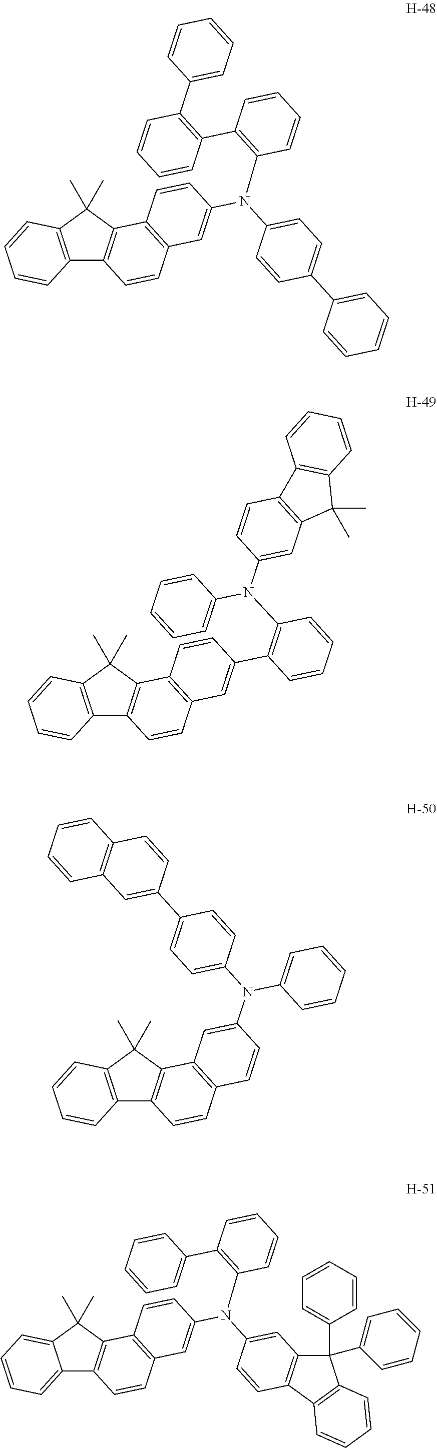 [SMILES: CC1(C)c2ccccc2-c2ccc3cc(N(c4ccc(-c5ccccc5)cc4)c4ccccc4-c4ccccc4-c4ccccc4)ccc3c21 ; CC1(C)c2ccccc2-c2ccc(N(c3ccccc3)c3ccccc3-c3ccc4c5c(ccc4c3)-c3ccccc3C5(C)C)cc21 ; CC1(C)c2ccccc2-c2ccc3ccc(N(c4ccccc4)c4ccc(-c5ccc6ccccc6c5)cc4)cc3c21 ; CC1(C)c2ccccc2-c2ccc3cc(N(c4ccc5c(c4)C(c4ccccc4)(c4ccccc4)c4ccccc4-5)c4ccccc4-c4ccccc4)ccc3c21]

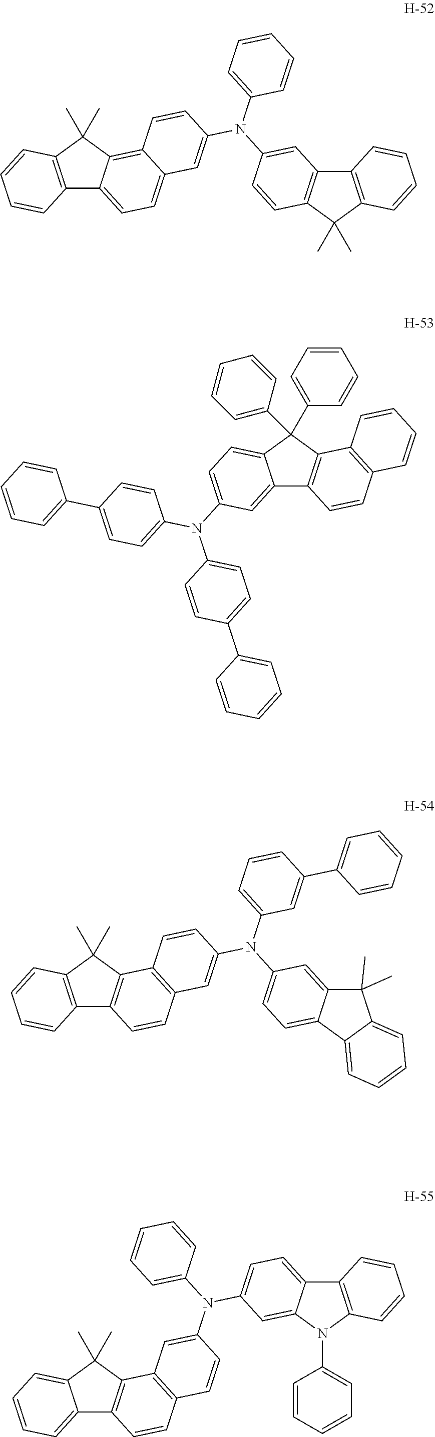 [SMILES: CC1(C)c2ccccc2-c2cc(N(c3ccccc3)c3ccc4c5c(ccc4c3)-c3ccccc3C5(C)C)ccc21 ; c1ccc(-c2ccc(N(c3ccc(-c4ccccc4)cc3)c3ccc4c(c3)-c3ccc5ccccc5c3C4(c3ccccc3)c3ccccc3)cc2)cc1 ; CC1(C)c2ccccc2-c2ccc(N(c3cccc(-c4ccccc4)c3)c3ccc4c5c(ccc4c3)-c3ccccc3C5(C)C)cc21 ; CC1(C)c2ccccc2-c2ccc3ccc(N(c4ccccc4)c4ccc5c6ccccc6n(-c6ccccc6)c5c4)cc3c21]

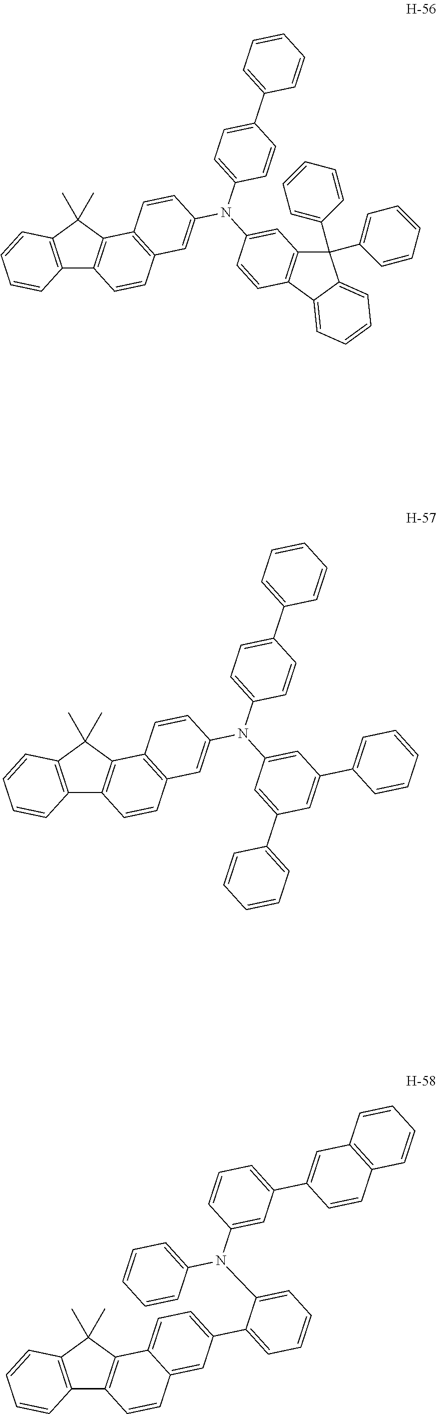 [SMILES: CC1(C)c2ccccc2-c2ccc3cc(N(c4ccc(-c5ccccc5)cc4)c4ccc5c(c4)C(c4ccccc4)(c4ccccc4)c4ccccc4-5)ccc3c21 ; CC1(C)c2ccccc2-c2ccc3cc(N(c4ccc(-c5ccccc5)cc4)c4cc(-c5ccccc5)cc(-c5ccccc5)c4)ccc3c21 ; CC1(C)c2ccccc2-c2ccc3cc(-c4ccccc4N(c4ccccc4)c4cccc(-c5ccc6ccccc6c5)c4)ccc3c21]

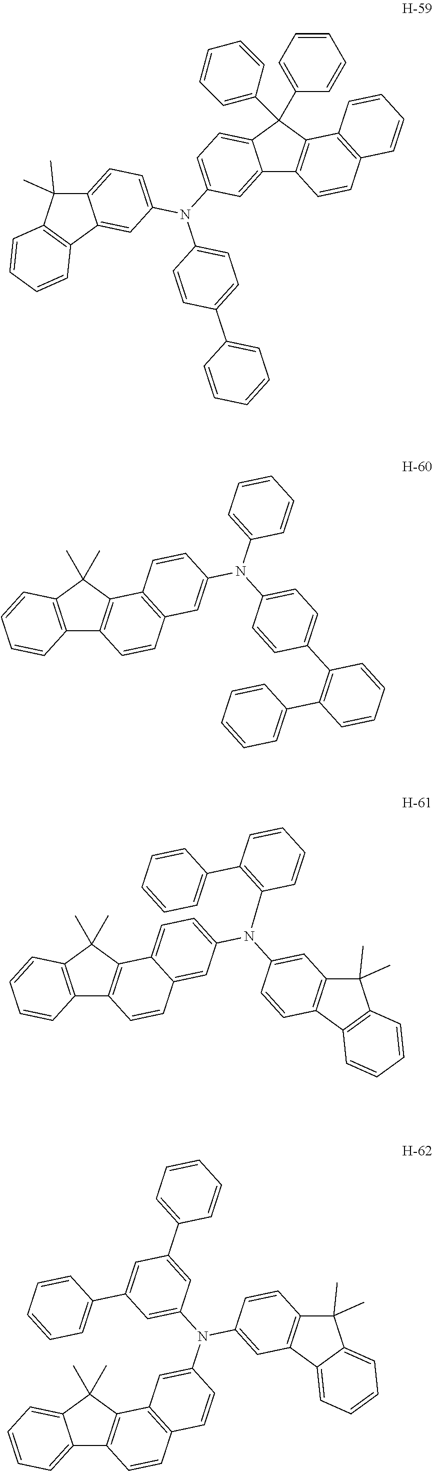 [SMILES: CC1(C)c2ccccc2-c2cc(N(c3ccc(-c4ccccc4)cc3)c3ccc4c(c3)-c3ccc5ccccc5c3C4(c3ccccc3)c3ccccc3)ccc21 ; CC1(C)c2ccccc2-c2ccc3cc(N(c4ccccc4)c4ccc(-c5ccccc5-c5ccccc5)cc4)ccc3c21 ; CC1(C)c2ccccc2-c2ccc(N(c3ccc4c5c(ccc4c3)-c3ccccc3C5(C)C)c3ccccc3-c3ccccc3)cc21 ; CC1(C)c2ccccc2-c2cc(N(c3cc(-c4ccccc4)cc(-c4ccccc4)c3)c3ccc4ccc5c(c4c3)C(C)(C)c3ccccc3-5)ccc21]

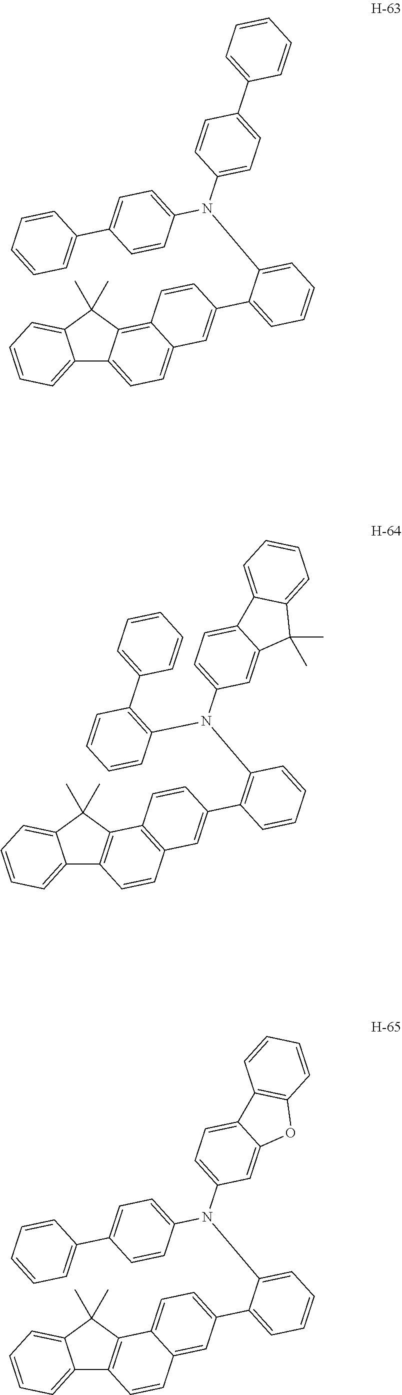 [SMILES: CC1(C)c2ccccc2-c2ccc3cc(-c4ccccc4N(c4ccc(-c5ccccc5)cc4)c4ccc(-c5ccccc5)cc4)ccc3c21 ; CC1(C)c2ccccc2-c2ccc(N(c3ccccc3-c3ccccc3)c3ccccc3-c3ccc4c5c(ccc4c3)-c3ccccc3C5(C)C)cc21 ; CC1(C)c2ccccc2-c2ccc3cc(-c4ccccc4N(c4ccc(-c5ccccc5)cc4)c4ccc5c(c4)oc4ccccc45)ccc3c21]

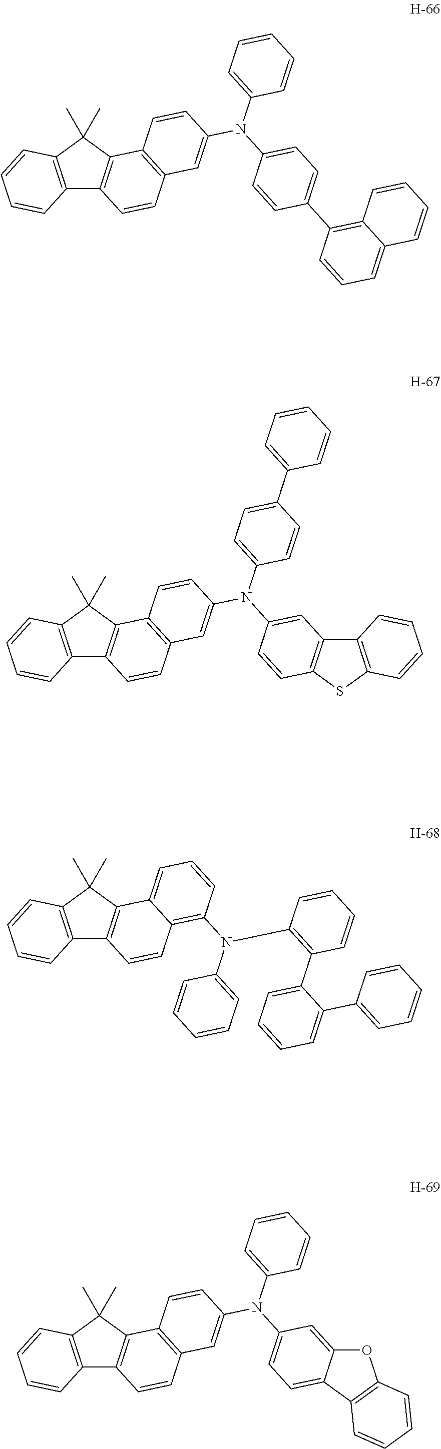 [SMILES: CC1(C)c2ccccc2-c2ccc3cc(N(c4ccccc4)c4ccc(-c5cccc6ccccc56)cc4)ccc3c21 ; CC1(C)c2ccccc2-c2ccc3cc(N(c4ccc(-c5ccccc5)cc4)c4ccc5sc6ccccc6c5c4)ccc3c21 ; CC1(C)c2ccccc2-c2ccc3c(N(c4ccccc4)c4ccccc4-c4ccccc4-c4ccccc4)cccc3c21 ; CC1(C)c2ccccc2-c2ccc3cc(N(c4ccccc4)c4ccc5c(c4)oc4ccccc45)ccc3c21]

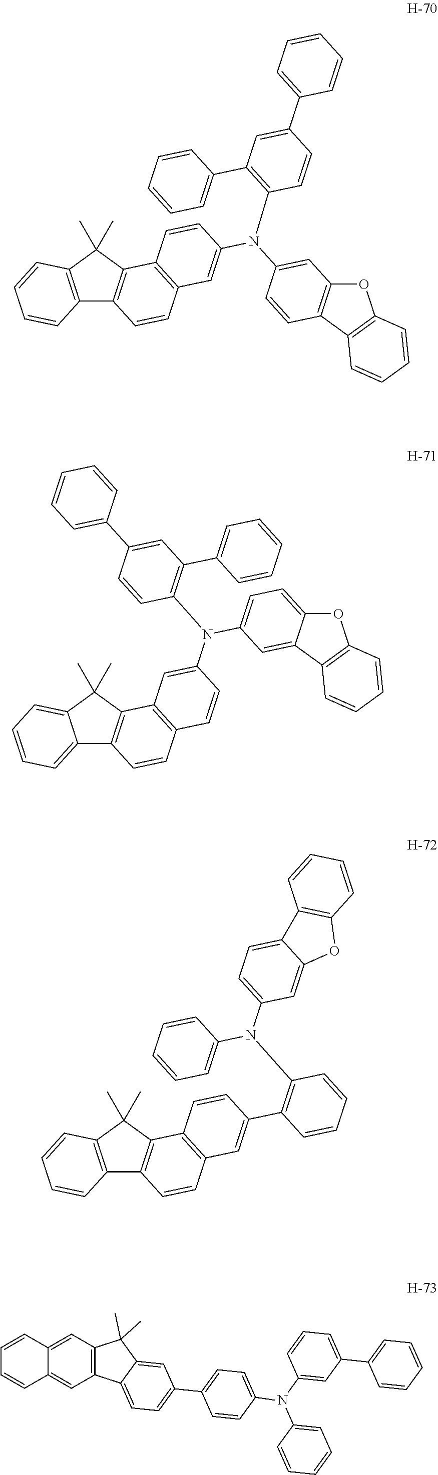 [SMILES: CC1(C)c2ccccc2-c2ccc3cc(N(c4ccc5c(c4)oc4ccccc45)c4ccc(-c5ccccc5)cc4-c4ccccc4)ccc3c21 ; CC1(C)c2ccccc2-c2ccc3ccc(N(c4ccc5oc6ccccc6c5c4)c4ccc(-c5ccccc5)cc4-c4ccccc4)cc3c21 ; CC1(C)c2ccccc2-c2ccc3cc(-c4ccccc4N(c4ccccc4)c4ccc5c(c4)oc4ccccc45)ccc3c21 ; CC1(C)c2cc(-c3ccc(N(c4ccccc4)c4cccc(-c5ccccc5)c4)cc3)ccc2-c2cc3ccccc3cc21]

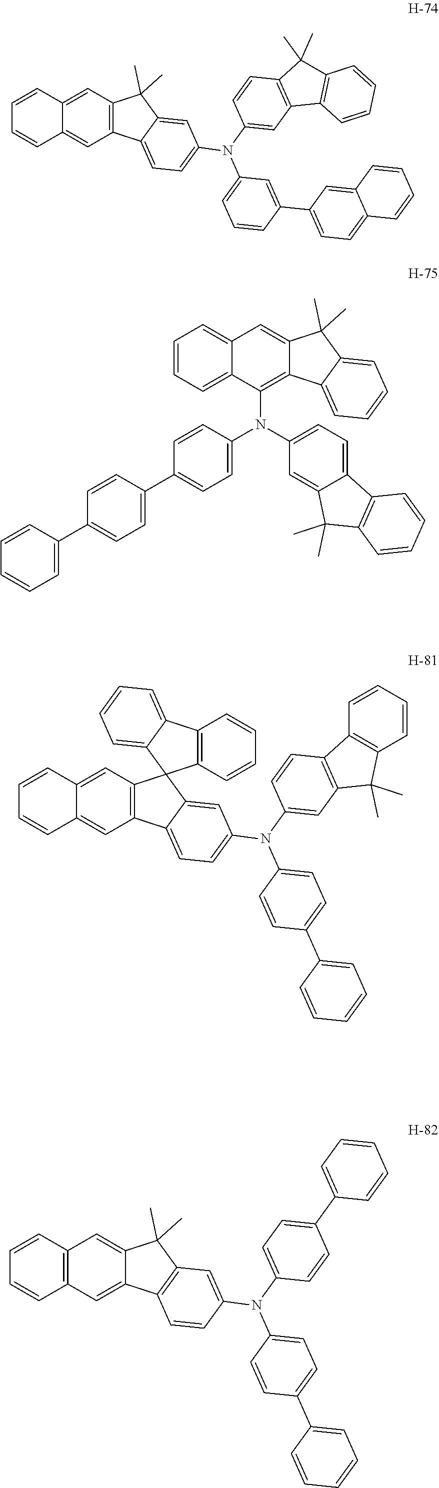 [SMILES: CC1(C)c2ccccc2-c2cc(N(c3cccc(-c4ccc5ccccc5c4)c3)c3ccc4c(c3)C(C)(C)c3cc5ccccc5cc3-4)ccc21 ; CC1(C)c2ccccc2-c2ccc(N(c3ccc(-c4ccc(-c5ccccc5)cc4)cc3)c3c4c(cc5ccccc35)C(C)(C)c3ccccc3-4)cc21 ; CC1(C)c2ccccc2-c2ccc(N(c3ccc(-c4ccccc4)cc3)c3ccc4c(c3)C3(c5ccccc5-c5ccccc53)c3cc5ccccc5cc3-4)cc21 ; CC1(C)c2cc(N(c3ccc(-c4ccccc4)cc3)c3ccc(-c4ccccc4)cc3)ccc2-c2cc3ccccc3cc21]

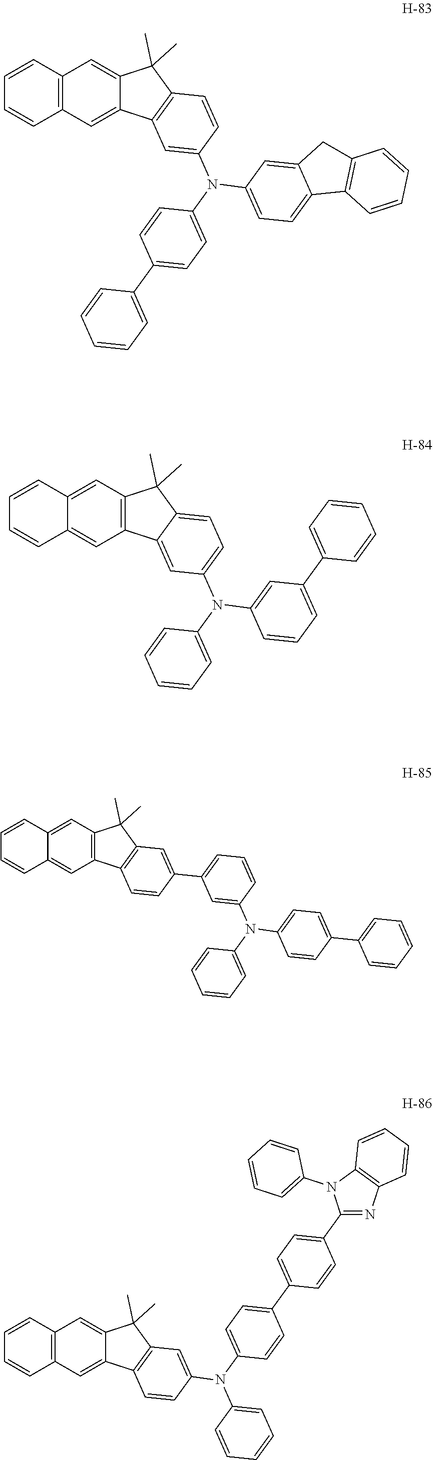 [SMILES: CC1(C)c2ccc(N(c3ccc(-c4ccccc4)cc3)c3ccc4c(c3)Cc3ccccc3-4)cc2-c2cc3ccccc3cc21 ; CC1(C)c2ccc(N(c3ccccc3)c3cccc(-c4ccccc4)c3)cc2-c2cc3ccccc3cc21 ; CC1(C)c2cc(-c3cccc(N(c4ccccc4)c4ccc(-c5ccccc5)cc4)c3)ccc2-c2cc3ccccc3cc21 ; CC1(C)c2cc(N(c3ccccc3)c3ccc(-c4ccc(-c5nc6ccccc6n5-c5ccccc5)cc4)cc3)ccc2-c2cc3ccccc3cc21]

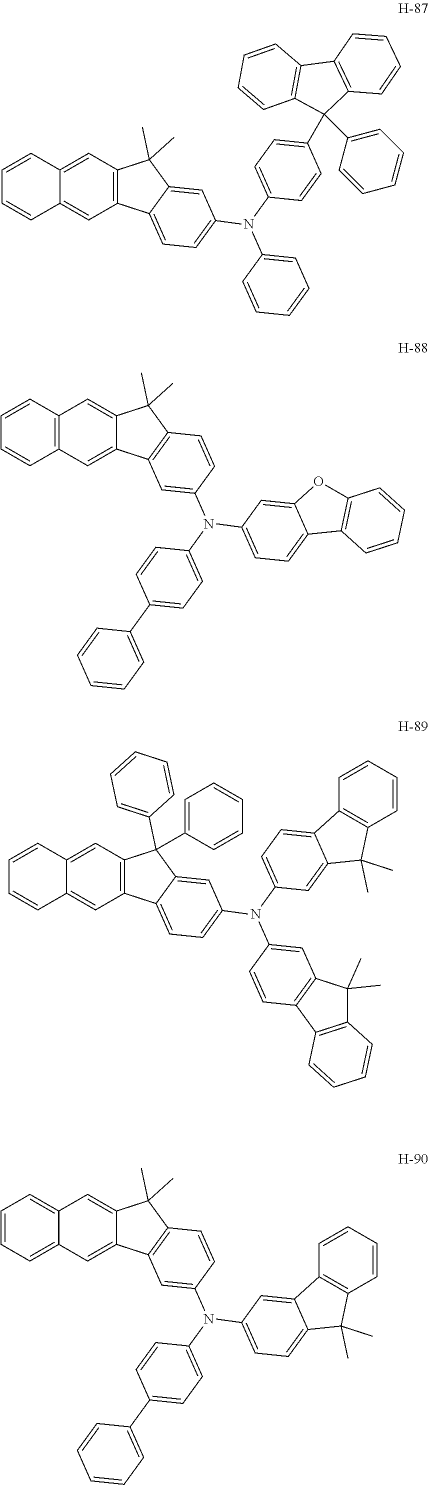 [SMILES: CC1(C)c2cc(N(c3ccccc3)c3ccc(C4(c5ccccc5)c5ccccc5-c5ccccc54)cc3)ccc2-c2cc3ccccc3cc21 ; CC1(C)c2ccc(N(c3ccc(-c4ccccc4)cc3)c3ccc4c(c3)oc3ccccc34)cc2-c2cc3ccccc3cc21 ; CC1(C)c2ccccc2-c2ccc(N(c3ccc4c(c3)C(C)(C)c3ccccc3-4)c3ccc4c(c3)C(c3ccccc3)(c3ccccc3)c3cc5ccccc5cc3-4)cc21 ; CC1(C)c2ccccc2-c2cc(N(c3ccc(-c4ccccc4)cc3)c3ccc4c(c3)-c3cc5ccccc5cc3C4(C)C)ccc21]

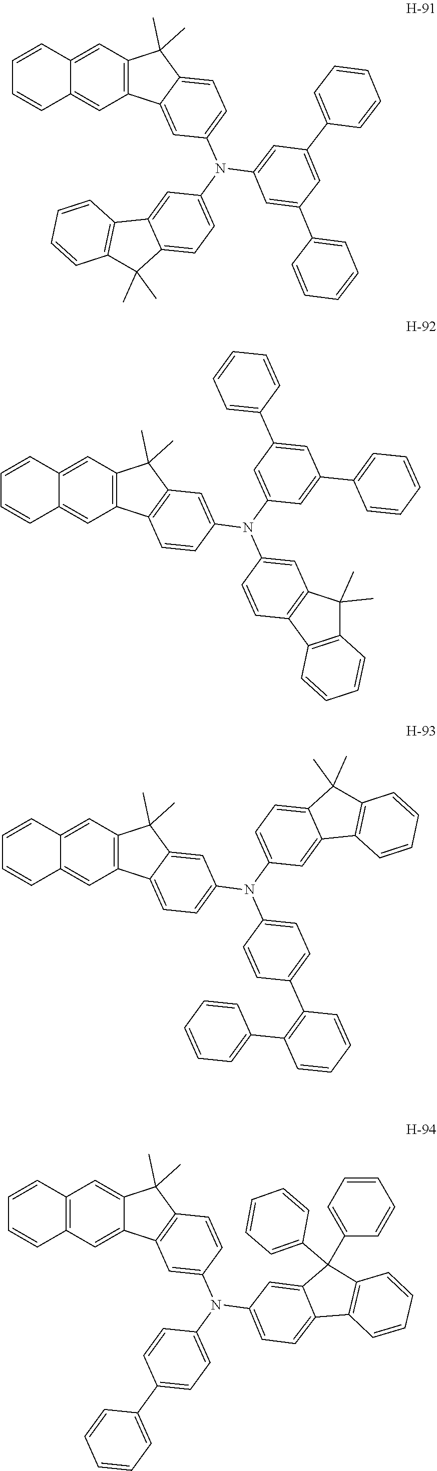 [SMILES: CC1(C)c2ccccc2-c2cc(N(c3cc(-c4ccccc4)cc(-c4ccccc4)c3)c3ccc4c(c3)-c3cc5ccccc5cc3C4(C)C)ccc21 ; CC1(C)c2ccccc2-c2ccc(N(c3cc(-c4ccccc4)cc(-c4ccccc4)c3)c3ccc4c(c3)C(C)(C)c3cc5ccccc5cc3-4)cc21 ; CC1(C)c2ccccc2-c2cc(N(c3ccc(-c4ccccc4-c4ccccc4)cc3)c3ccc4c(c3)C(C)(C)c3cc5ccccc5cc3-4)ccc21 ; CC1(C)c2ccc(N(c3ccc(-c4ccccc4)cc3)c3ccc4c(c3)C(c3ccccc3)(c3ccccc3)c3ccccc3-4)cc2-c2cc3ccccc3cc21]

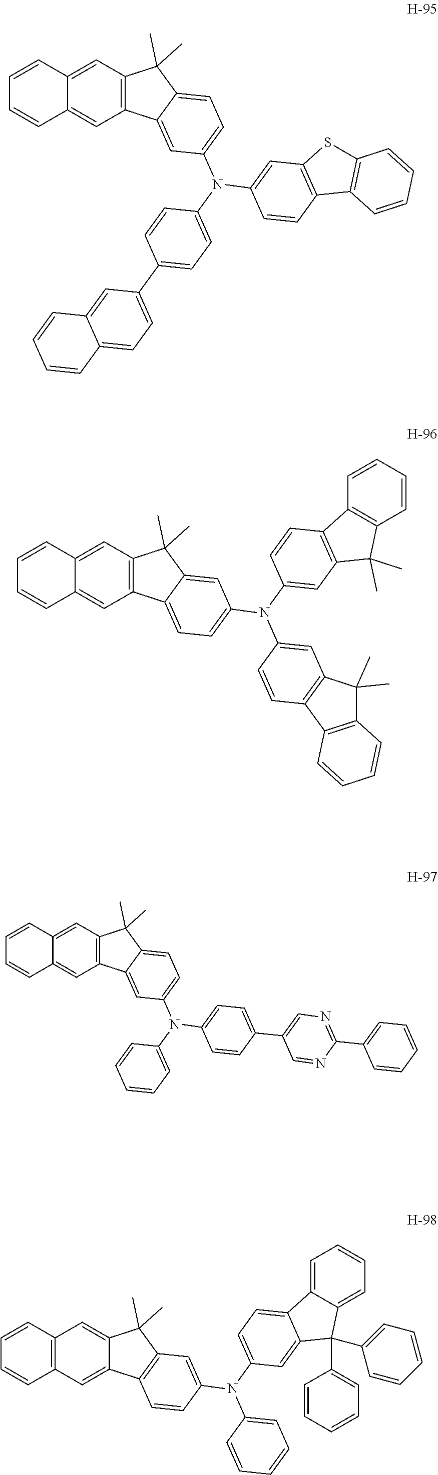 [SMILES: CC1(C)c2ccc(N(c3ccc(-c4ccc5ccccc5c4)cc3)c3ccc4c(c3)sc3ccccc34)cc2-c2cc3ccccc3cc21 ; CC1(C)c2ccccc2-c2ccc(N(c3ccc4c(c3)C(C)(C)c3ccccc3-4)c3ccc4c(c3)C(C)(C)c3cc5ccccc5cc3-4)cc21 ; CC1(C)c2ccc(N(c3ccccc3)c3ccc(-c4cnc(-c5ccccc5)nc4)cc3)cc2-c2cc3ccccc3cc21 ; CC1(C)c2cc(N(c3ccccc3)c3ccc4c(c3)C(c3ccccc3)(c3ccccc3)c3ccccc3-4)ccc2-c2cc3ccccc3cc21]

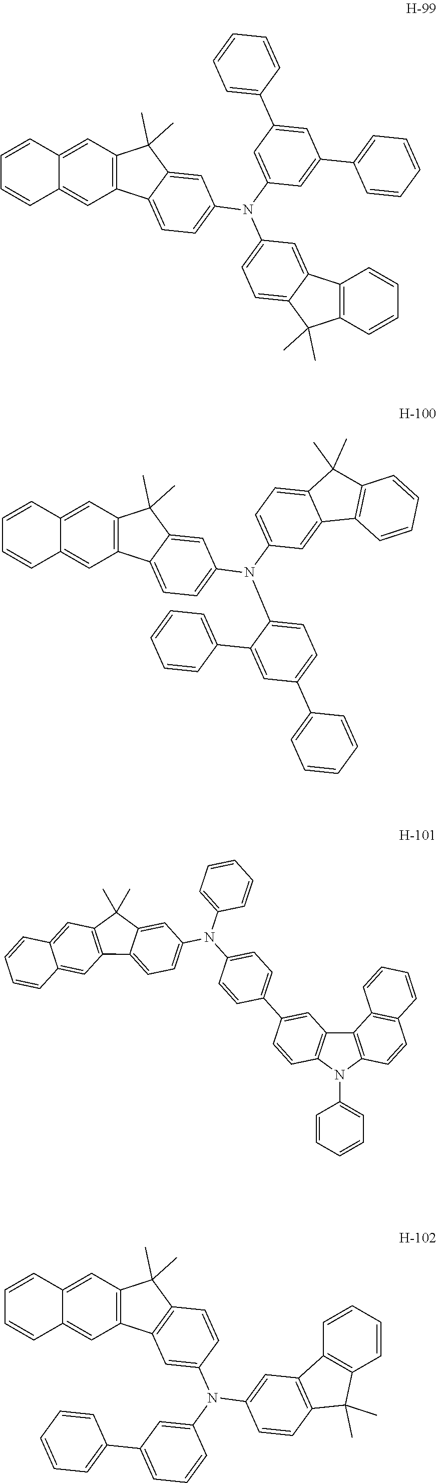 [SMILES: CC1(C)c2ccccc2-c2cc(N(c3cc(-c4ccccc4)cc(-c4ccccc4)c3)c3ccc4c(c3)C(C)(C)c3cc5ccccc5cc3-4)ccc21 ; CC1(C)c2ccccc2-c2cc(N(c3ccc4c(c3)C(C)(C)c3cc5ccccc5cc3-4)c3ccc(-c4ccccc4)cc3-c3ccccc3)ccc21 ; CC1(C)c2cc(N(c3ccccc3)c3ccc(-c4ccc5c(c4)c4c6ccccc6ccc4n5-c4ccccc4)cc3)ccc2-c2cc3ccccc3cc21 ; CC1(C)c2ccccc2-c2cc(N(c3cccc(-c4ccccc4)c3)c3ccc4c(c3)-c3cc5ccccc5cc3C4(C)C)ccc21]

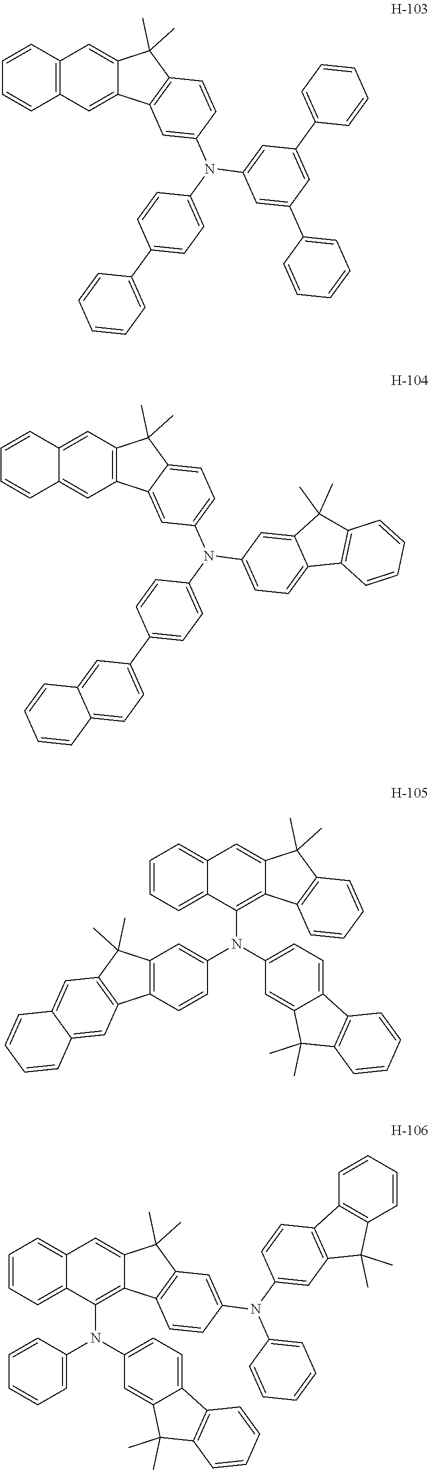 [SMILES: CC1(C)c2ccc(N(c3ccc(-c4ccccc4)cc3)c3cc(-c4ccccc4)cc(-c4ccccc4)c3)cc2-c2cc3ccccc3cc21 ; CC1(C)c2ccccc2-c2ccc(N(c3ccc(-c4ccc5ccccc5c4)cc3)c3ccc4c(c3)-c3cc5ccccc5cc3C4(C)C)cc21 ; CC1(C)c2ccccc2-c2ccc(N(c3ccc4c(c3)C(C)(C)c3cc5ccccc5cc3-4)c3c4c(cc5ccccc35)C(C)(C)c3ccccc3-4)cc21 ; CC1(C)c2ccccc2-c2ccc(N(c3ccccc3)c3ccc4c(c3)C(C)(C)c3cc5ccccc5c(N(c5ccccc5)c5ccc6c(c5)C(C)(C)c5ccccc5-6)c3-4)cc21]

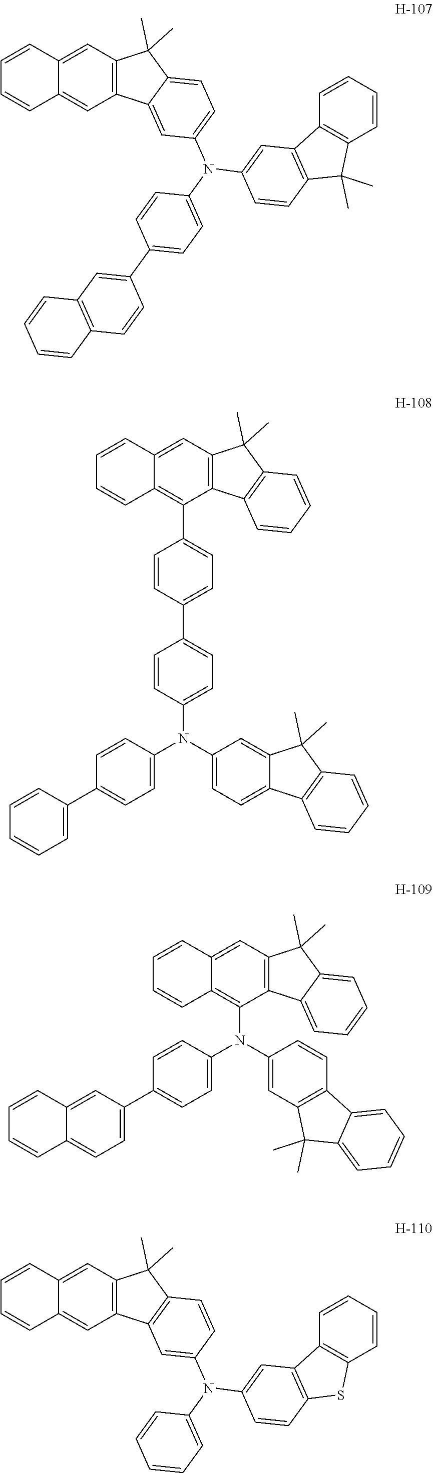 [SMILES: CC1(C)c2ccccc2-c2cc(N(c3ccc(-c4ccc5ccccc5c4)cc3)c3ccc4c(c3)-c3cc5ccccc5cc3C4(C)C)ccc21 ; CC1(C)c2ccccc2-c2ccc(N(c3ccc(-c4ccccc4)cc3)c3ccc(-c4ccc(-c5c6c(cc7ccccc57)C(C)(C)c5ccccc5-6)cc4)cc3)cc21 ; CC1(C)c2ccccc2-c2ccc(N(c3ccc(-c4ccc5ccccc5c4)cc3)c3c4c(cc5ccccc35)C(C)(C)c3ccccc3-4)cc21 ; CC1(C)c2ccc(N(c3ccccc3)c3ccc4sc5ccccc5c4c3)cc2-c2cc3ccccc3cc21]

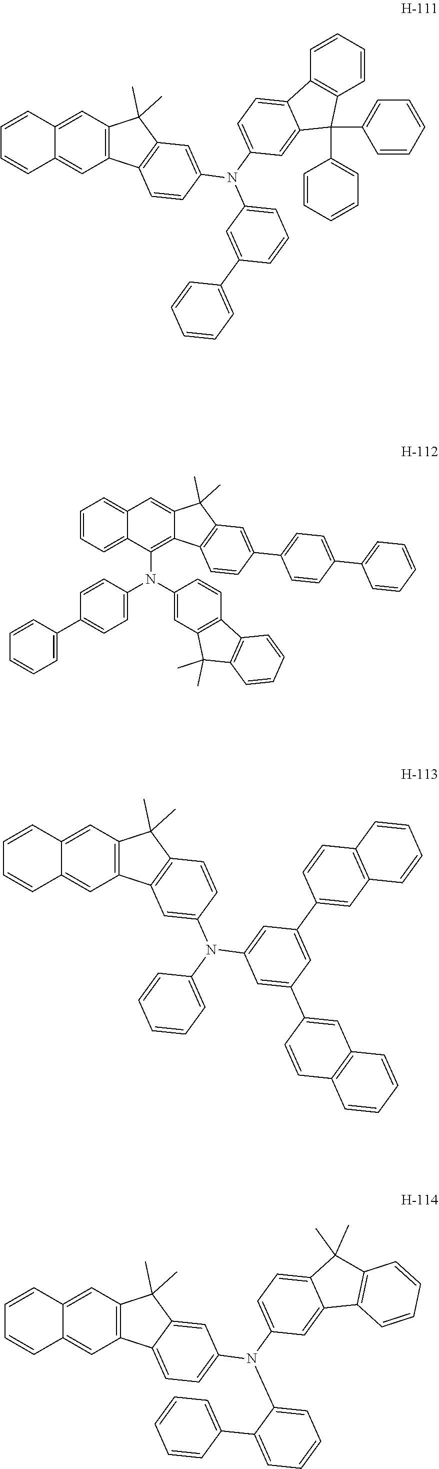

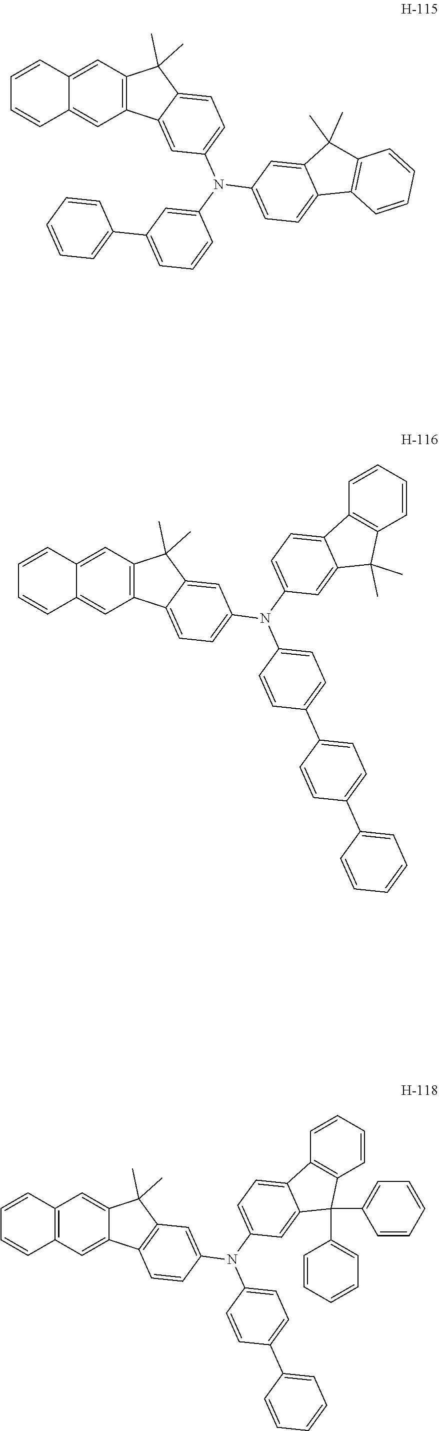

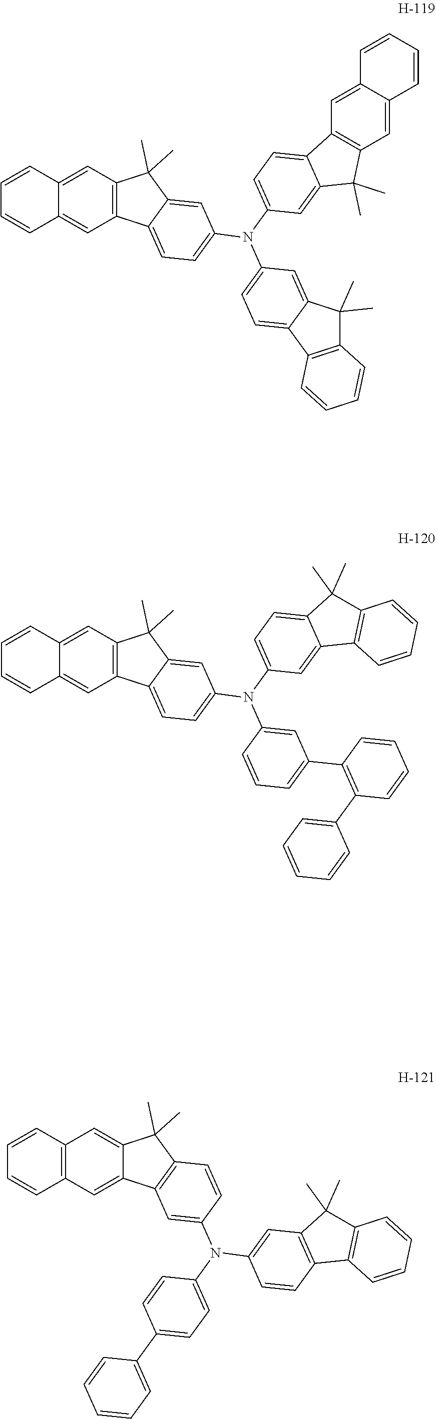 [SMILES: CC1(C)c2ccccc2-c2ccc(N(c3ccc4c(c3)C(C)(C)c3cc5ccccc5cc3-4)c3ccc4c(c3)C(C)(C)c3cc5ccccc5cc3-4)cc21 ; CC1(C)c2ccccc2-c2cc(N(c3cccc(-c4ccccc4-c4ccccc4)c3)c3ccc4c(c3)C(C)(C)c3cc5ccccc5cc3-4)ccc21 ; CC1(C)c2ccccc2-c2ccc(N(c3ccc(-c4ccccc4)cc3)c3ccc4c(c3)-c3cc5ccccc5cc3C4(C)C)cc21]

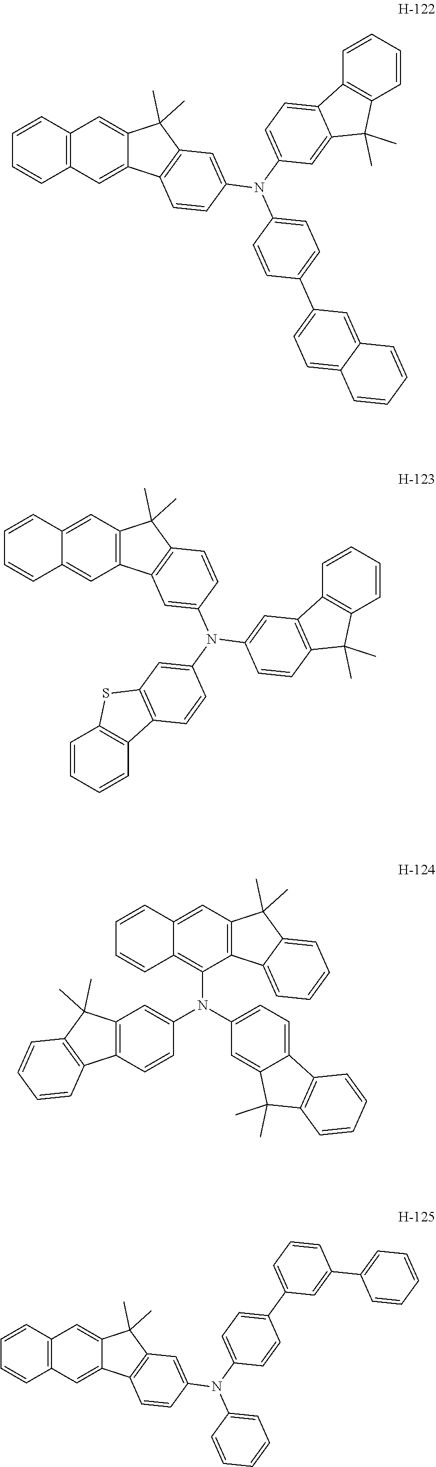 [SMILES: CC1(C)c2ccccc2-c2ccc(N(c3ccc(-c4ccc5ccccc5c4)cc3)c3ccc4c(c3)C(C)(C)c3cc5ccccc5cc3-4)cc21 ; CC1(C)c2ccccc2-c2cc(N(c3ccc4c(c3)-c3cc5ccccc5cc3C4(C)C)c3ccc4c(c3)sc3ccccc34)ccc21 ; CC1(C)c2ccccc2-c2ccc(N(c3ccc4c(c3)C(C)(C)c3ccccc3-4)c3c4c(cc5ccccc35)C(C)(C)c3ccccc3-4)cc21 ; CC1(C)c2cc(N(c3ccccc3)c3ccc(-c4cccc(-c5ccccc5)c4)cc3)ccc2-c2cc3ccccc3cc21]

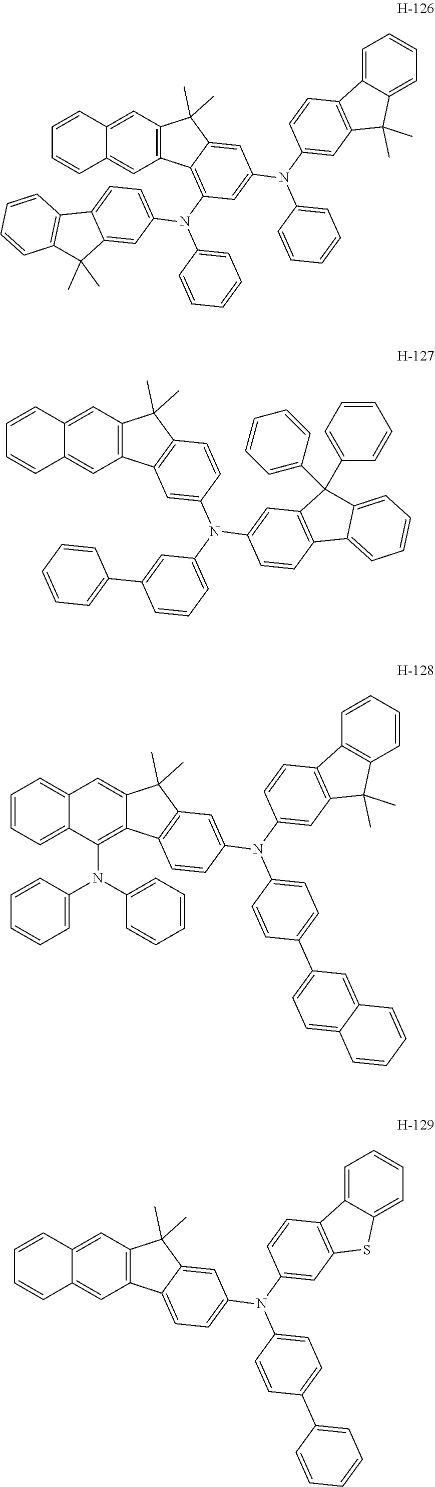 [SMILES: CC1(C)c2ccccc2-c2ccc(N(c3ccccc3)c3cc(N(c4ccccc4)c4ccc5c(c4)C(C)(C)c4ccccc4-5)c4c(c3)C(C)(C)c3cc5ccccc5cc3-4)cc21 ; CC1(C)c2ccc(N(c3cccc(-c4ccccc4)c3)c3ccc4c(c3)C(c3ccccc3)(c3ccccc3)c3ccccc3-4)cc2-c2cc3ccccc3cc21 ; CC1(C)c2ccccc2-c2ccc(N(c3ccc(-c4ccc5ccccc5c4)cc3)c3ccc4c(c3)C(C)(C)c3cc5ccccc5c(N(c5ccccc5)c5ccccc5)c3-4)cc21 ; CC1(C)c2cc(N(c3ccc(-c4ccccc4)cc3)c3ccc4c(c3)sc3ccccc34)ccc2-c2cc3ccccc3cc21]

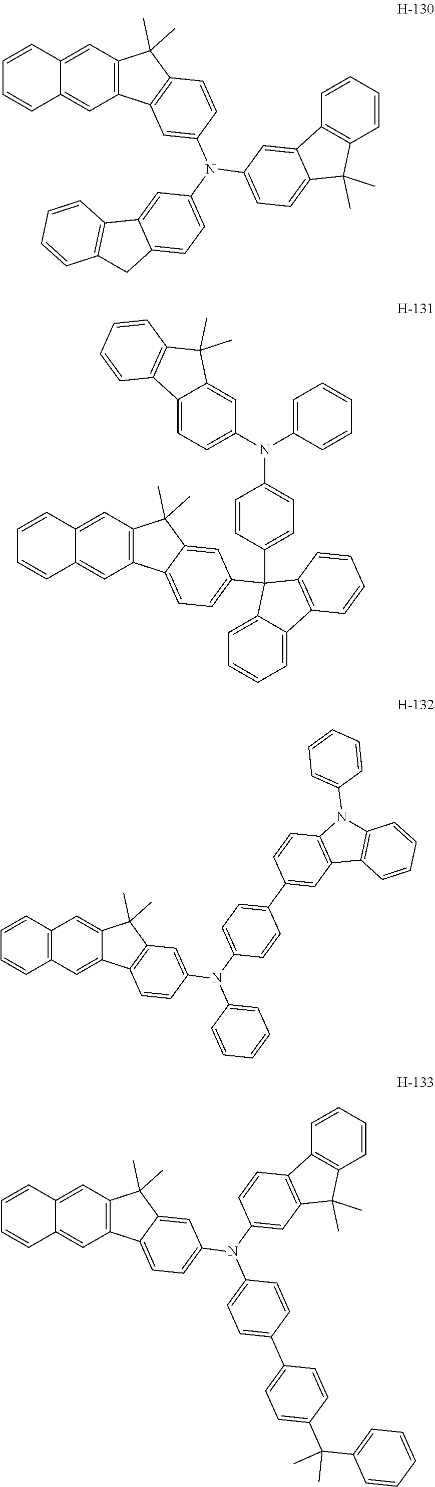 [SMILES: CC1(C)c2ccccc2-c2cc(N(c3ccc4c(c3)-c3ccccc3C4)c3ccc4c(c3)-c3cc5ccccc5cc3C4(C)C)ccc21 ; CC1(C)c2ccccc2-c2ccc(N(c3ccccc3)c3ccc(C4(c5ccc6c(c5)C(C)(C)c5cc7ccccc7cc5-6)c5ccccc5-c5ccccc54)cc3)cc21 ; CC1(C)c2cc(N(c3ccccc3)c3ccc(-c4ccc5c(c4)c4ccccc4n5-c4ccccc4)cc3)ccc2-c2cc3ccccc3cc21 ; CC(C)(c1ccccc1)c1ccc(-c2ccc(N(c3ccc4c(c3)C(C)(C)c3ccccc3-4)c3ccc4c(c3)C(C)(C)c3cc5ccccc5cc3-4)cc2)cc1]

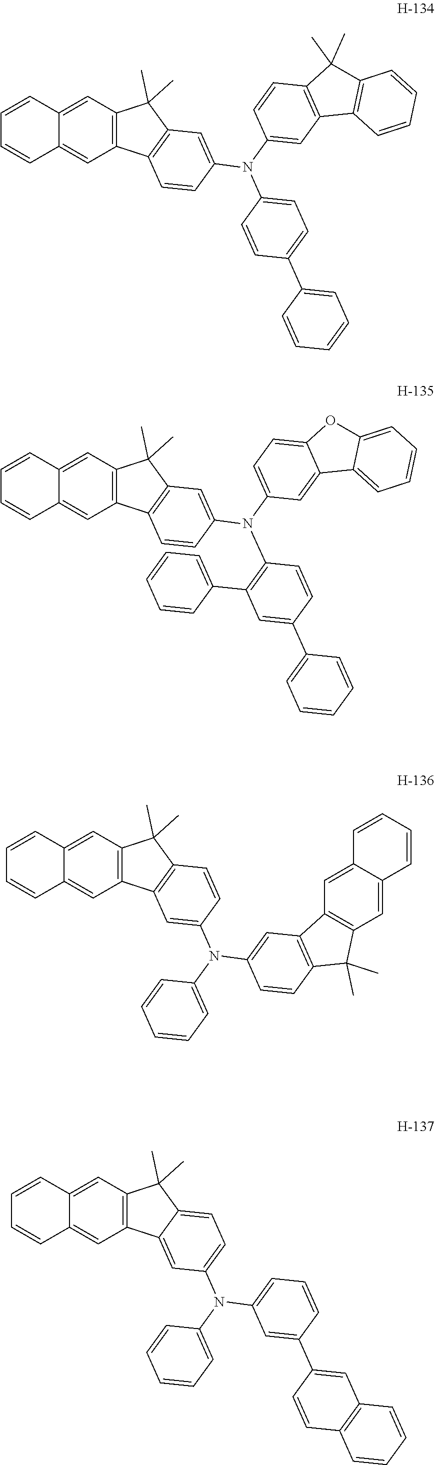 [SMILES: CC1(C)c2ccccc2-c2cc(N(c3ccc(-c4ccccc4)cc3)c3ccc4c(c3)C(C)(C)c3cc5ccccc5cc3-4)ccc21 ; CC1(C)c2cc(N(c3ccc4oc5ccccc5c4c3)c3ccc(-c4ccccc4)cc3-c3ccccc3)ccc2-c2cc3ccccc3cc21 ; CC1(C)c2ccc(N(c3ccccc3)c3ccc4c(c3)-c3cc5ccccc5cc3C4(C)C)cc2-c2cc3ccccc3cc21 ; CC1(C)c2ccc(N(c3ccccc3)c3cccc(-c4ccc5ccccc5c4)c3)cc2-c2cc3ccccc3cc21]

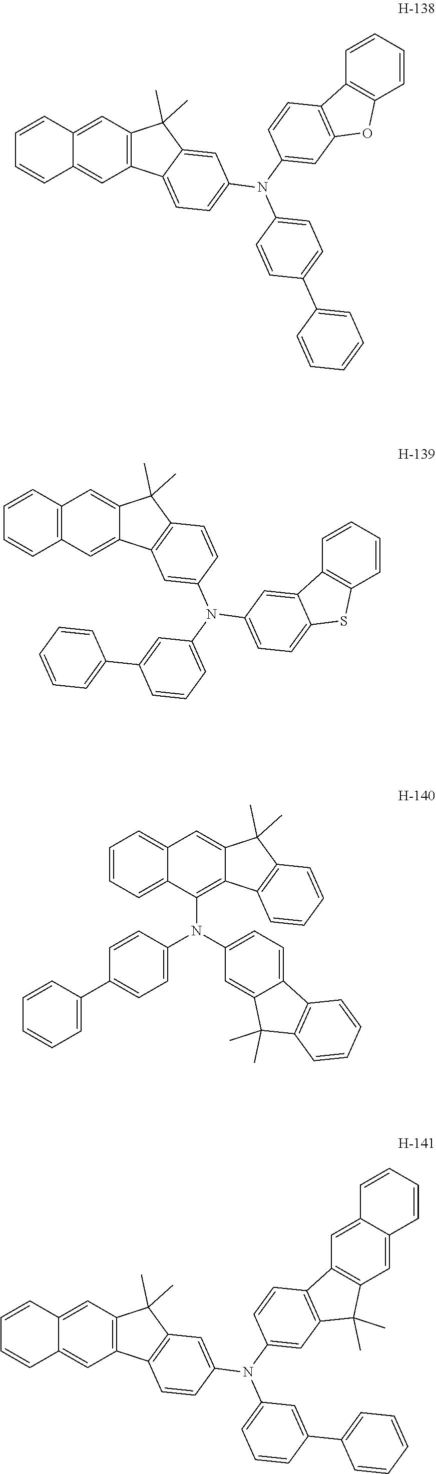 [SMILES: CC1(C)c2cc(N(c3ccc(-c4ccccc4)cc3)c3ccc4c(c3)oc3ccccc34)ccc2-c2cc3ccccc3cc21 ; CC1(C)c2ccc(N(c3cccc(-c4ccccc4)c3)c3ccc4sc5ccccc5c4c3)cc2-c2cc3ccccc3cc21 ; CC1(C)c2ccccc2-c2ccc(N(c3ccc(-c4ccccc4)cc3)c3c4c(cc5ccccc35)C(C)(C)c3ccccc3-4)cc21 ; CC1(C)c2cc(N(c3cccc(-c4ccccc4)c3)c3ccc4c(c3)C(C)(C)c3cc5ccccc5cc3-4)ccc2-c2cc3ccccc3cc21]

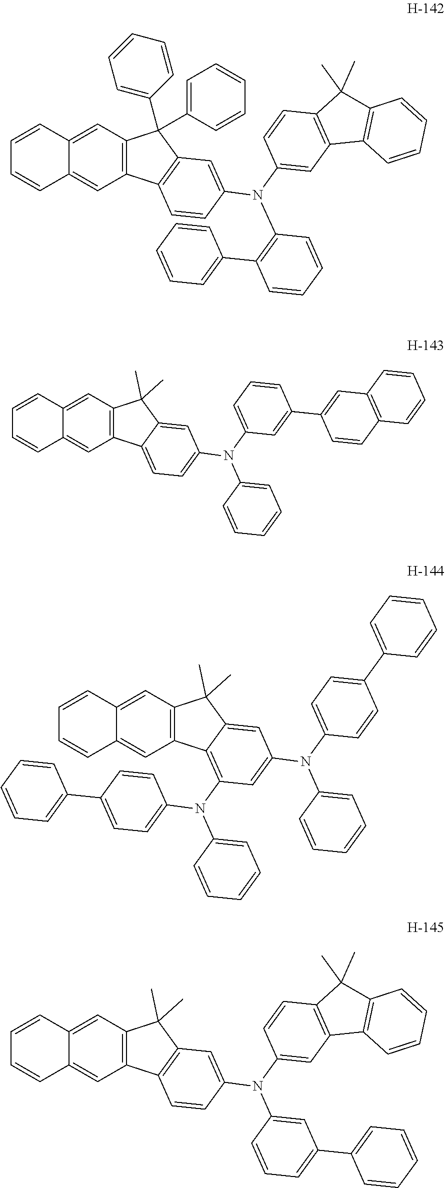 [SMILES: CC1(C)c2ccccc2-c2cc(N(c3ccc4c(c3)C(c3ccccc3)(c3ccccc3)c3cc5ccccc5cc3-4)c3ccccc3-c3ccccc3)ccc21 ; CC1(C)c2cc(N(c3ccccc3)c3cccc(-c4ccc5ccccc5c4)c3)ccc2-c2cc3ccccc3cc21 ; CC1(C)c2cc3ccccc3cc2-c2c(N(c3ccccc3)c3ccc(-c4ccccc4)cc3)cc(N(c3ccccc3)c3ccc(-c4ccccc4)cc3)cc21 ; CC1(C)c2ccccc2-c2cc(N(c3cccc(-c4ccccc4)c3)c3ccc4c(c3)C(C)(C)c3cc5ccccc5cc3-4)ccc21]

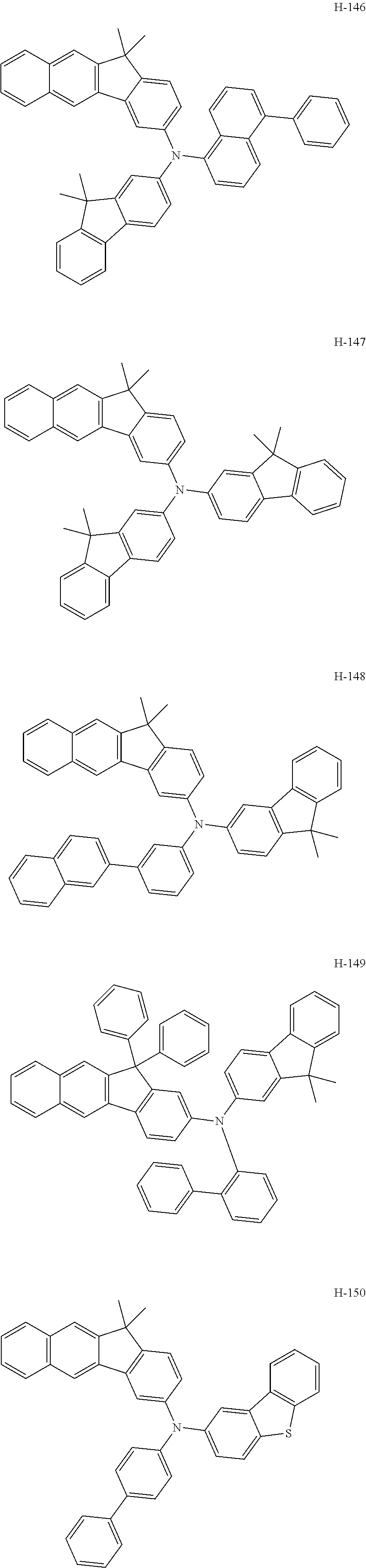 [SMILES: CC1(C)c2ccccc2-c2ccc(N(c3ccc4c(c3)-c3cc5ccccc5cc3C4(C)C)c3cccc4c(-c5ccccc5)cccc34)cc21 ; CC1(C)c2ccccc2-c2ccc(N(c3ccc4c(c3)-c3cc5ccccc5cc3C4(C)C)c3ccc4c(c3)C(C)(C)c3ccccc3-4)cc21 ; CC1(C)c2ccccc2-c2cc(N(c3cccc(-c4ccc5ccccc5c4)c3)c3ccc4c(c3)-c3cc5ccccc5cc3C4(C)C)ccc21 ; CC1(C)c2ccccc2-c2ccc(N(c3ccc4c(c3)C(c3ccccc3)(c3ccccc3)c3cc5ccccc5cc3-4)c3ccccc3-c3ccccc3)cc21 ; CC1(C)c2ccc(N(c3ccc(-c4ccccc4)cc3)c3ccc4sc5ccccc5c4c3)cc2-c2cc3ccccc3cc21]

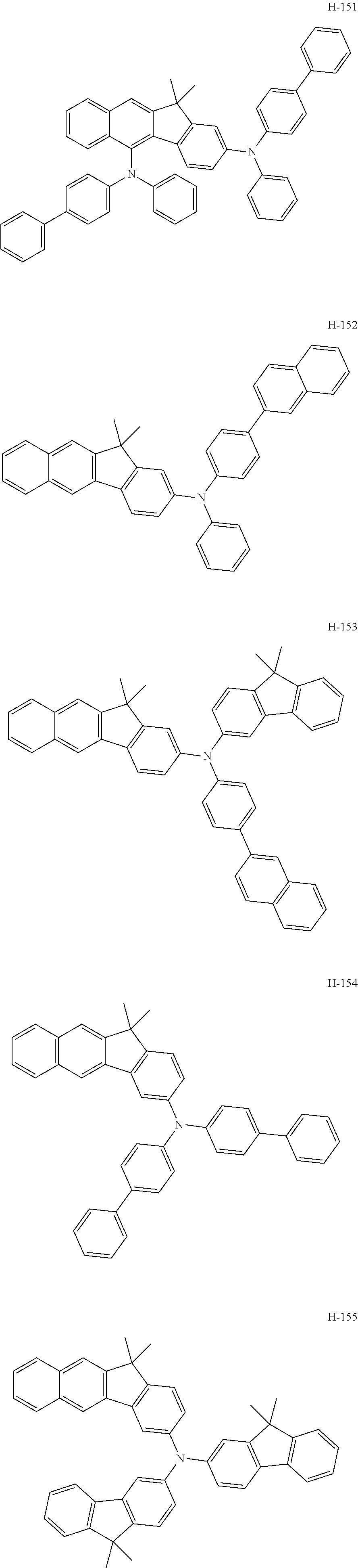 [SMILES: CC1(C)c2cc(N(c3ccccc3)c3ccc(-c4ccccc4)cc3)ccc2-c2c1cc1ccccc1c2N(c1ccccc1)c1ccc(-c2ccccc2)cc1 ; CC1(C)c2cc(N(c3ccccc3)c3ccc(-c4ccc5ccccc5c4)cc3)ccc2-c2cc3ccccc3cc21 ; CC1(C)c2ccccc2-c2cc(N(c3ccc(-c4ccc5ccccc5c4)cc3)c3ccc4c(c3)C(C)(C)c3cc5ccccc5cc3-4)ccc21 ; CC1(C)c2ccc(N(c3ccc(-c4ccccc4)cc3)c3ccc(-c4ccccc4)cc3)cc2-c2cc3ccccc3cc21 ; CC1(C)c2ccccc2-c2cc(N(c3ccc4c(c3)-c3cc5ccccc5cc3C4(C)C)c3ccc4c(c3)C(C)(C)c3ccccc3-4)ccc21]

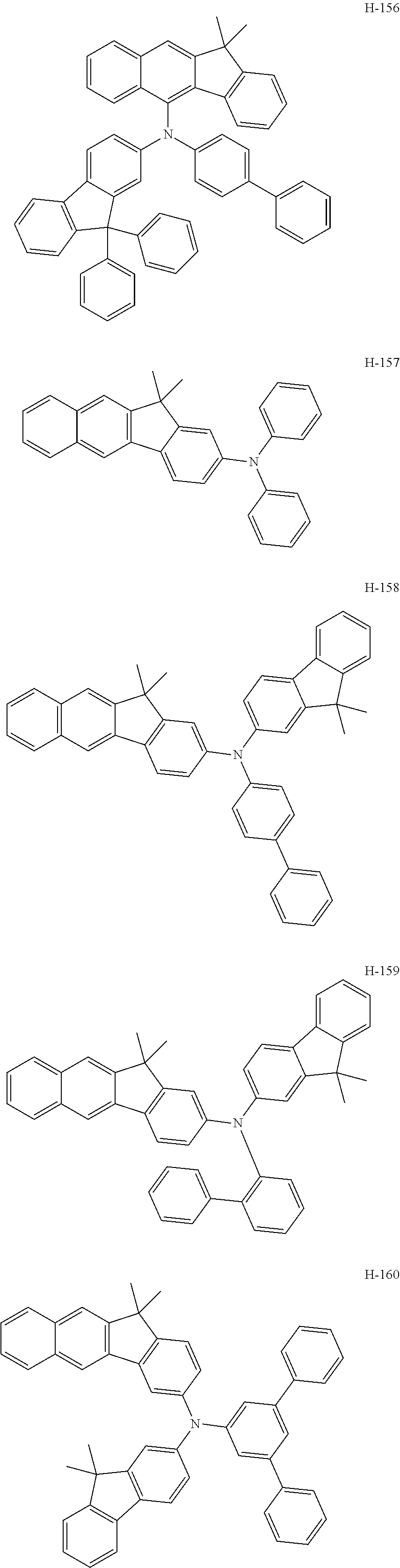 [SMILES: CC1(C)c2ccccc2-c2c1cc1ccccc1c2N(c1ccc(-c2ccccc2)cc1)c1ccc2c(c1)C(c1ccccc1)(c1ccccc1)c1ccccc1-2 ; CC1(C)c2cc(N(c3ccccc3)c3ccccc3)ccc2-c2cc3ccccc3cc21 ; CC1(C)c2ccccc2-c2ccc(N(c3ccc(-c4ccccc4)cc3)c3ccc4c(c3)C(C)(C)c3cc5ccccc5cc3-4)cc21 ; CC1(C)c2ccccc2-c2ccc(N(c3ccc4c(c3)C(C)(C)c3cc5ccccc5cc3-4)c3ccccc3-c3ccccc3)cc21 ; CC1(C)c2ccccc2-c2ccc(N(c3cc(-c4ccccc4)cc(-c4ccccc4)c3)c3ccc4c(c3)-c3cc5ccccc5cc3C4(C)C)cc21]

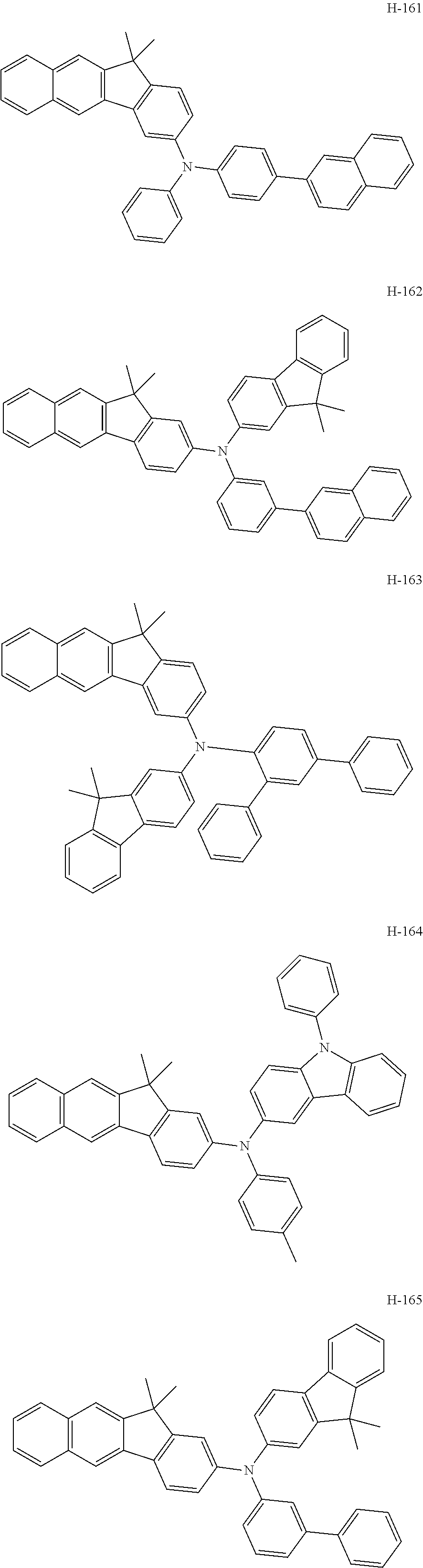 [SMILES: CC1(C)c2ccc(N(c3ccccc3)c3ccc(-c4ccc5ccccc5c4)cc3)cc2-c2cc3ccccc3cc21 ; CC1(C)c2ccccc2-c2ccc(N(c3cccc(-c4ccc5ccccc5c4)c3)c3ccc4c(c3)C(C)(C)c3cc5ccccc5cc3-4)cc21 ; CC1(C)c2ccccc2-c2ccc(N(c3ccc4c(c3)-c3cc5ccccc5cc3C4(C)C)c3ccc(-c4ccccc4)cc3-c3ccccc3)cc21 ; Cc1ccc(N(c2ccc3c(c2)C(C)(C)c2cc4ccccc4cc2-3)c2ccc3c(c2)c2ccccc2n3-c2ccccc2)cc1 ; CC1(C)c2ccccc2-c2ccc(N(c3cccc(-c4ccccc4)c3)c3ccc4c(c3)C(C)(C)c3cc5ccccc5cc3-4)cc21]

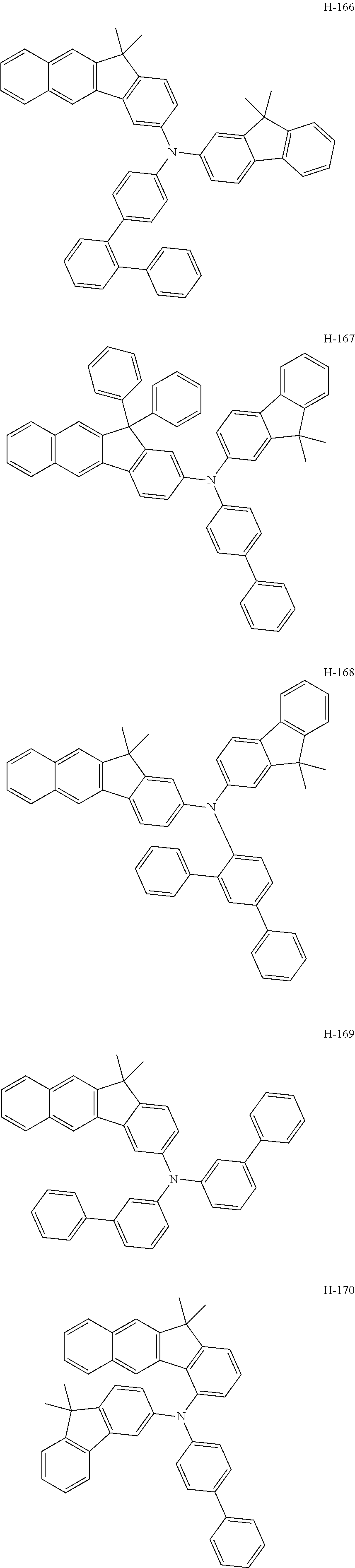 [SMILES: CC1(C)c2ccccc2-c2ccc(N(c3ccc(-c4ccccc4-c4ccccc4)cc3)c3ccc4c(c3)-c3cc5ccccc5cc3C4(C)C)cc21 ; CC1(C)c2ccccc2-c2ccc(N(c3ccc(-c4ccccc4)cc3)c3ccc4c(c3)C(c3ccccc3)(c3ccccc3)c3cc5ccccc5cc3-4)cc21 ; CC1(C)c2ccccc2-c2ccc(N(c3ccc4c(c3)C(C)(C)c3cc5ccccc5cc3-4)c3ccc(-c4ccccc4)cc3-c3ccccc3)cc21 ; CC1(C)c2ccc(N(c3cccc(-c4ccccc4)c3)c3cccc(-c4ccccc4)c3)cc2-c2cc3ccccc3cc21 ; CC1(C)c2ccccc2-c2cc(N(c3ccc(-c4ccccc4)cc3)c3cccc4c3-c3cc5ccccc5cc3C4(C)C)ccc21]

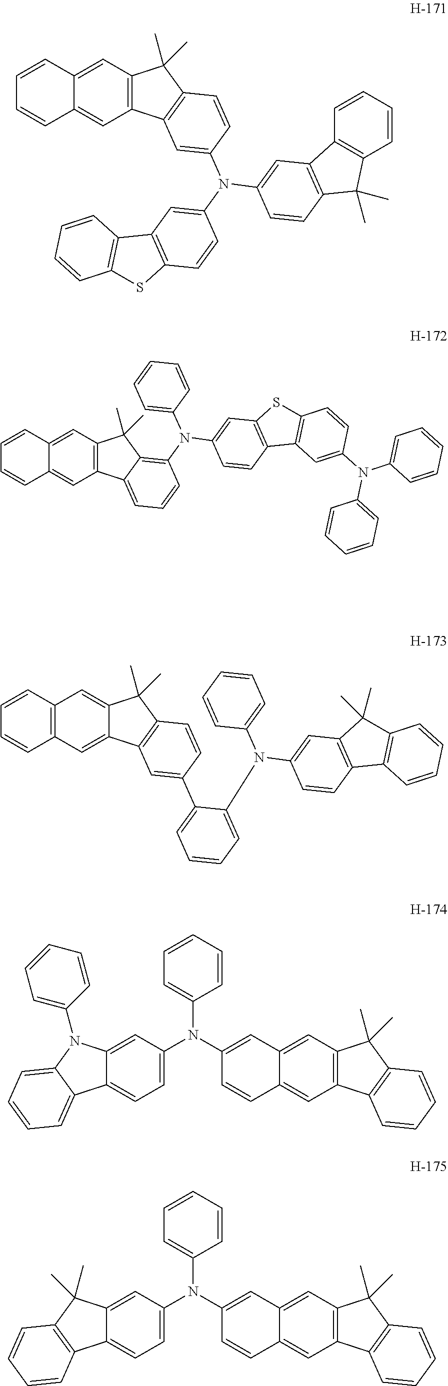 [SMILES: CC1(C)c2ccccc2-c2cc(N(c3ccc4c(c3)-c3cc5ccccc5cc3C4(C)C)c3ccc4sc5ccccc5c4c3)ccc21 ; CC1(C)c2cc3ccccc3cc2-c2cccc(N(c3ccccc3)c3ccc4c(c3)sc3ccc(N(c5ccccc5)c5ccccc5)cc34)c21 ; CC1(C)c2ccccc2-c2ccc(N(c3ccccc3)c3ccccc3-c3ccc4c(c3)-c3cc5ccccc5cc3C4(C)C)cc21 ; CC1(C)c2ccccc2-c2cc3ccc(N(c4ccccc4)c4ccc5c6ccccc6n(-c6ccccc6)c5c4)cc3cc21 ; CC1(C)c2ccccc2-c2ccc(N(c3ccccc3)c3ccc4cc5c(cc4c3)C(C)(C)c3ccccc3-5)cc21]

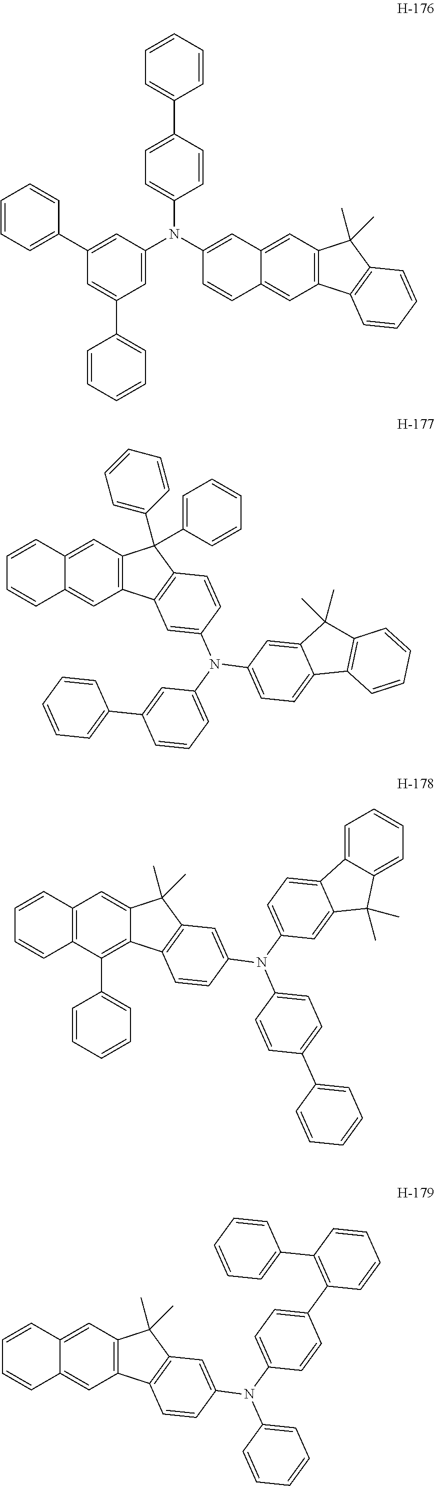 [SMILES: CC1(C)c2ccccc2-c2cc3ccc(N(c4ccc(-c5ccccc5)cc4)c4cc(-c5ccccc5)cc(-c5ccccc5)c4)cc3cc21 ; CC1(C)c2ccccc2-c2ccc(N(c3cccc(-c4ccccc4)c3)c3ccc4c(c3)-c3cc5ccccc5cc3C4(c3ccccc3)c3ccccc3)cc21 ; CC1(C)c2ccccc2-c2ccc(N(c3ccc(-c4ccccc4)cc3)c3ccc4c(c3)C(C)(C)c3cc5ccccc5c(-c5ccccc5)c3-4)cc21 ; CC1(C)c2cc(N(c3ccccc3)c3ccc(-c4ccccc4-c4ccccc4)cc3)ccc2-c2cc3ccccc3cc21]

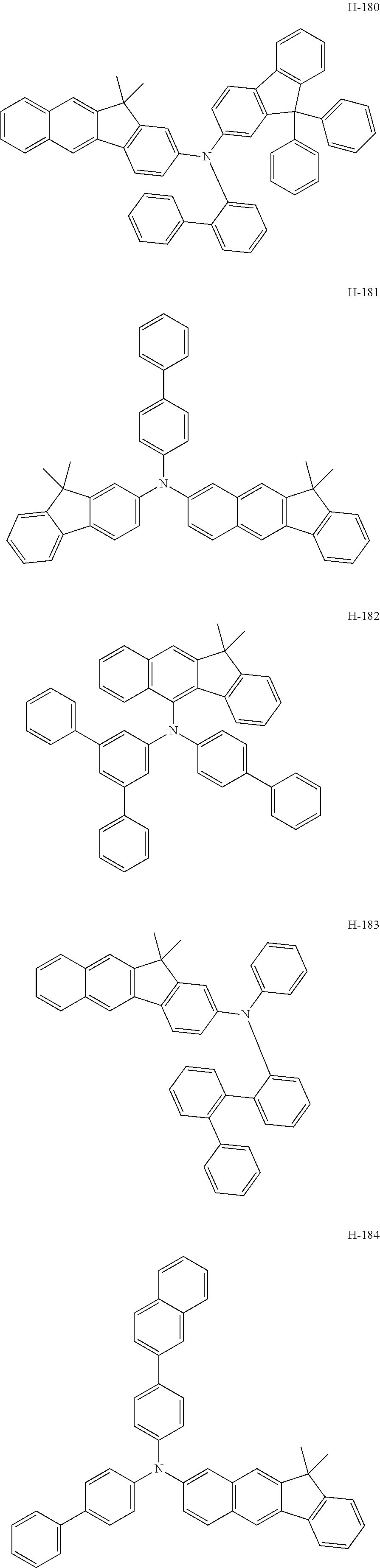 [SMILES: CC1(C)c2cc(N(c3ccc4c(c3)C(c3ccccc3)(c3ccccc3)c3ccccc3-4)c3ccccc3-c3ccccc3)ccc2-c2cc3ccccc3cc21 ; CC1(C)c2ccccc2-c2ccc(N(c3ccc(-c4ccccc4)cc3)c3ccc4cc5c(cc4c3)C(C)(C)c3ccccc3-5)cc21 ; CC1(C)c2ccccc2-c2c1cc1ccccc1c2N(c1ccc(-c2ccccc2)cc1)c1cc(-c2ccccc2)cc(-c2ccccc2)c1 ; CC1(C)c2cc(N(c3ccccc3)c3ccccc3-c3ccccc3-c3ccccc3)ccc2-c2cc3ccccc3cc21 ; CC1(C)c2ccccc2-c2cc3ccc(N(c4ccc(-c5ccccc5)cc4)c4ccc(-c5ccc6ccccc6c5)cc4)cc3cc21]

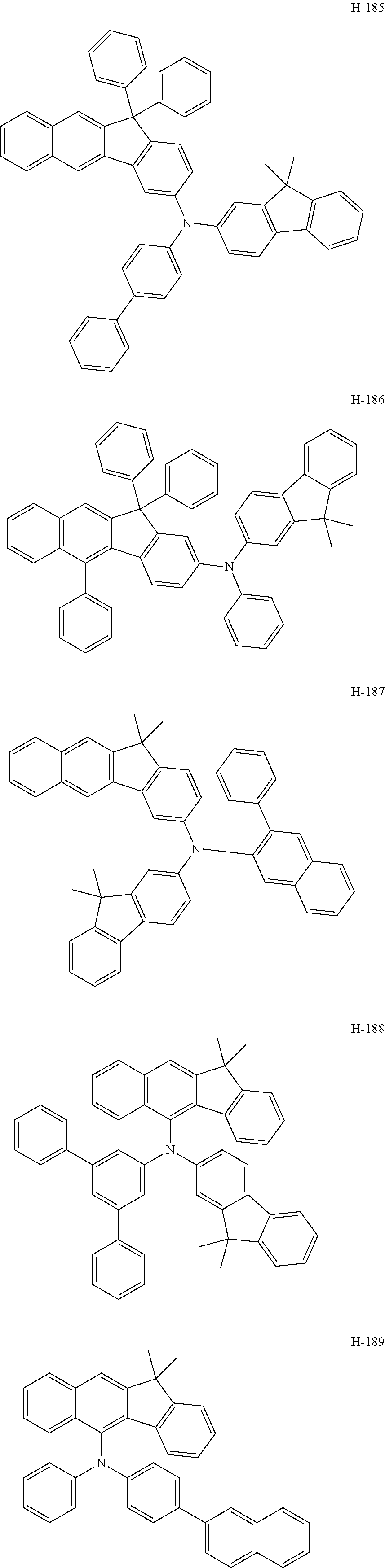 [SMILES: CC1(C)c2ccccc2-c2ccc(N(c3ccc(-c4ccccc4)cc3)c3ccc4c(c3)-c3cc5ccccc5cc3C4(c3ccccc3)c3ccccc3)cc21 ; CC1(C)c2ccccc2-c2ccc(N(c3ccccc3)c3ccc4c(c3)C(c3ccccc3)(c3ccccc3)c3cc5ccccc5c(-c5ccccc5)c3-4)cc21 ; CC1(C)c2ccccc2-c2ccc(N(c3ccc4c(c3)-c3cc5ccccc5cc3C4(C)C)c3cc4ccccc4cc3-c3ccccc3)cc21 ; CC1(C)c2ccccc2-c2ccc(N(c3cc(-c4ccccc4)cc(-c4ccccc4)c3)c3c4c(cc5ccccc35)C(C)(C)c3ccccc3-4)cc21 ; CC1(C)c2ccccc2-c2c1cc1ccccc1c2N(c1ccccc1)c1ccc(-c2ccc3ccccc3c2)cc1]

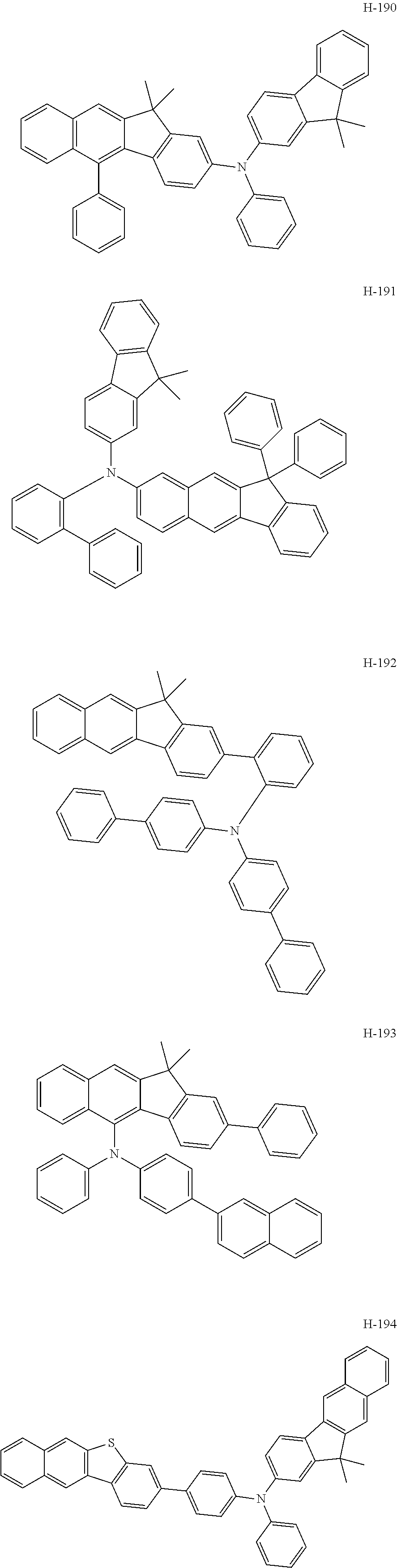 [SMILES: CC1(C)c2ccccc2-c2ccc(N(c3ccccc3)c3ccc4c(c3)C(C)(C)c3cc5ccccc5c(-c5ccccc5)c3-4)cc21 ; CC1(C)c2ccccc2-c2ccc(N(c3ccc4cc5c(cc4c3)C(c3ccccc3)(c3ccccc3)c3ccccc3-5)c3ccccc3-c3ccccc3)cc21 ; CC1(C)c2cc(-c3ccccc3N(c3ccc(-c4ccccc4)cc3)c3ccc(-c4ccccc4)cc3)ccc2-c2cc3ccccc3cc21 ; CC1(C)c2cc(-c3ccccc3)ccc2-c2c1cc1ccccc1c2N(c1ccccc1)c1ccc(-c2ccc3ccccc3c2)cc1 ; CC1(C)c2cc(N(c3ccccc3)c3ccc(-c4ccc5c(c4)sc4cc6ccccc6cc45)cc3)ccc2-c2cc3ccccc3cc21]

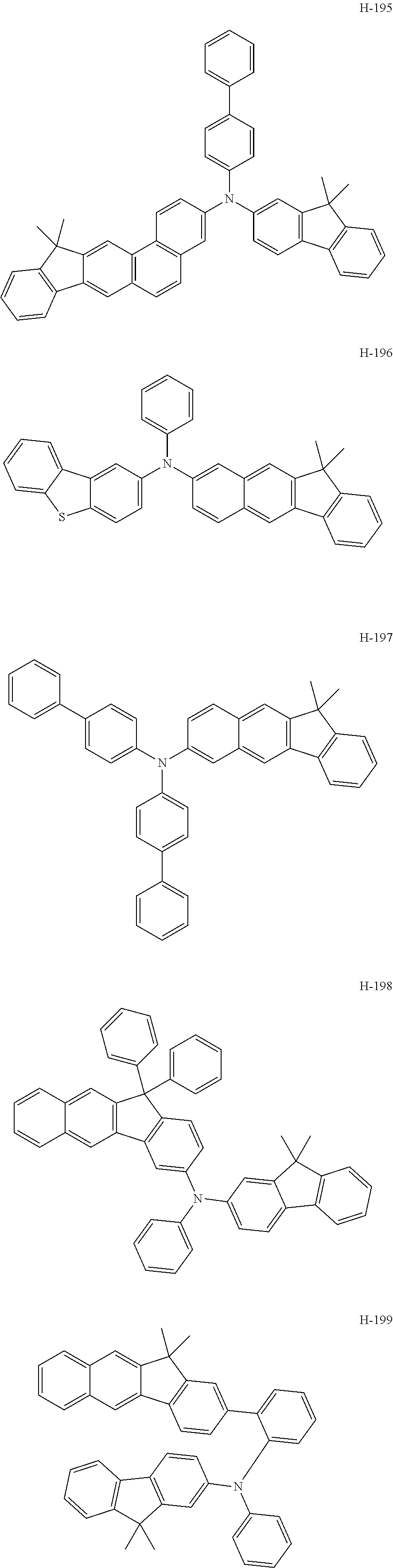 [SMILES: CC1(C)c2ccccc2-c2ccc(N(c3ccc(-c4ccccc4)cc3)c3ccc4c(ccc5cc6c(cc54)C(C)(C)c4ccccc4-6)c3)cc21 ; CC1(C)c2ccccc2-c2cc3ccc(N(c4ccccc4)c4ccc5sc6ccccc6c5c4)cc3cc21 ; CC1(C)c2ccccc2-c2cc3cc(N(c4ccc(-c5ccccc5)cc4)c4ccc(-c5ccccc5)cc4)ccc3cc21 ; CC1(C)c2ccccc2-c2ccc(N(c3ccccc3)c3ccc4c(c3)-c3cc5ccccc5cc3C4(c3ccccc3)c3ccccc3)cc21 ; CC1(C)c2ccccc2-c2ccc(N(c3ccccc3)c3ccccc3-c3ccc4c(c3)C(C)(C)c3cc5ccccc5cc3-4)cc21]

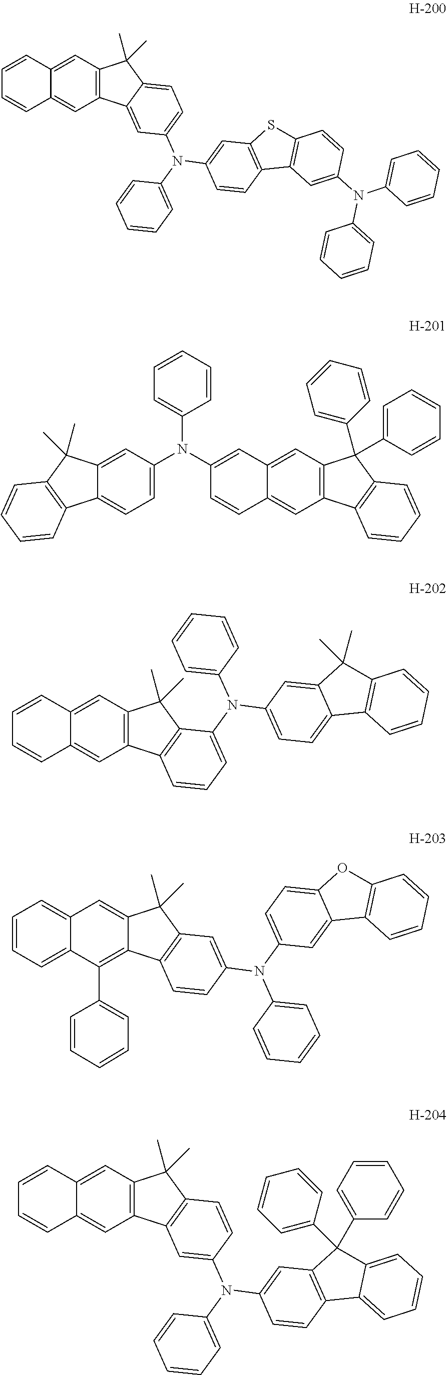 [SMILES: CC1(C)c2ccc(N(c3ccccc3)c3ccc4c(c3)sc3ccc(N(c5ccccc5)c5ccccc5)cc34)cc2-c2cc3ccccc3cc21 ; CC1(C)c2ccccc2-c2ccc(N(c3ccccc3)c3ccc4cc5c(cc4c3)C(c3ccccc3)(c3ccccc3)c3ccccc3-5)cc21 ; CC1(C)c2ccccc2-c2ccc(N(c3ccccc3)c3cccc4c3C(C)(C)c3cc5ccccc5cc3-4)cc21 ; CC1(C)c2cc(N(c3ccccc3)c3ccc4oc5ccccc5c4c3)ccc2-c2c1cc1ccccc1c2-c1ccccc1 ; CC1(C)c2ccc(N(c3ccccc3)c3ccc4c(c3)C(c3ccccc3)(c3ccccc3)c3ccccc3-4)cc2-c2cc3ccccc3cc21]

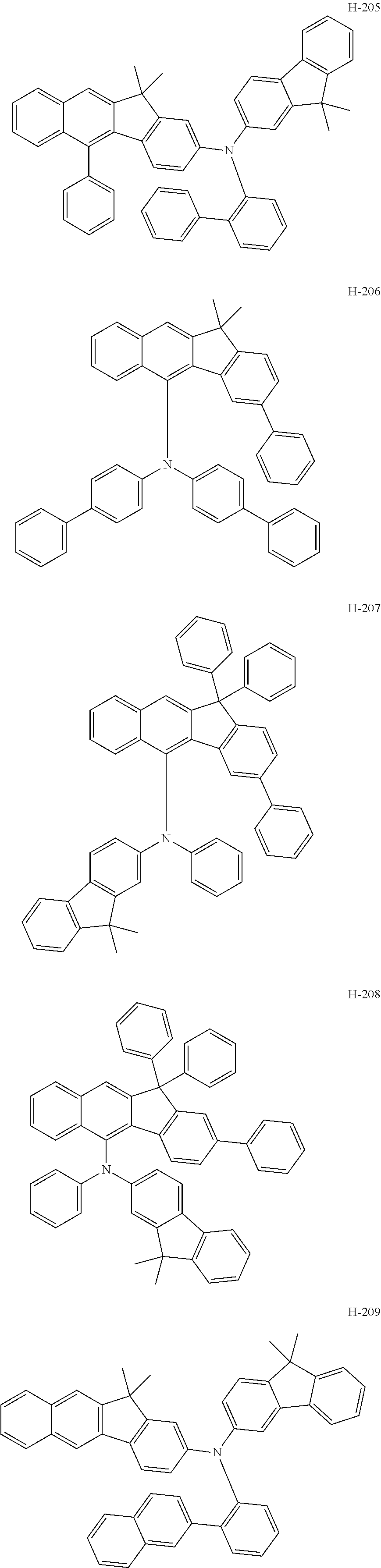 [SMILES: CC1(C)c2ccccc2-c2ccc(N(c3ccc4c(c3)C(C)(C)c3cc5ccccc5c(-c5ccccc5)c3-4)c3ccccc3-c3ccccc3)cc21 ; CC1(C)c2ccc(-c3ccccc3)cc2-c2c1cc1ccccc1c2N(c1ccc(-c2ccccc2)cc1)c1ccc(-c2ccccc2)cc1 ; CC1(C)c2ccccc2-c2ccc(N(c3ccccc3)c3c4c(cc5ccccc35)C(c3ccccc3)(c3ccccc3)c3ccc(-c5ccccc5)cc3-4)cc21 ; CC1(C)c2ccccc2-c2ccc(N(c3ccccc3)c3c4c(cc5ccccc35)C(c3ccccc3)(c3ccccc3)c3cc(-c5ccccc5)ccc3-4)cc21 ; CC1(C)c2ccccc2-c2cc(N(c3ccc4c(c3)C(C)(C)c3cc5ccccc5cc3-4)c3ccccc3-c3ccc4ccccc4c3)ccc21]

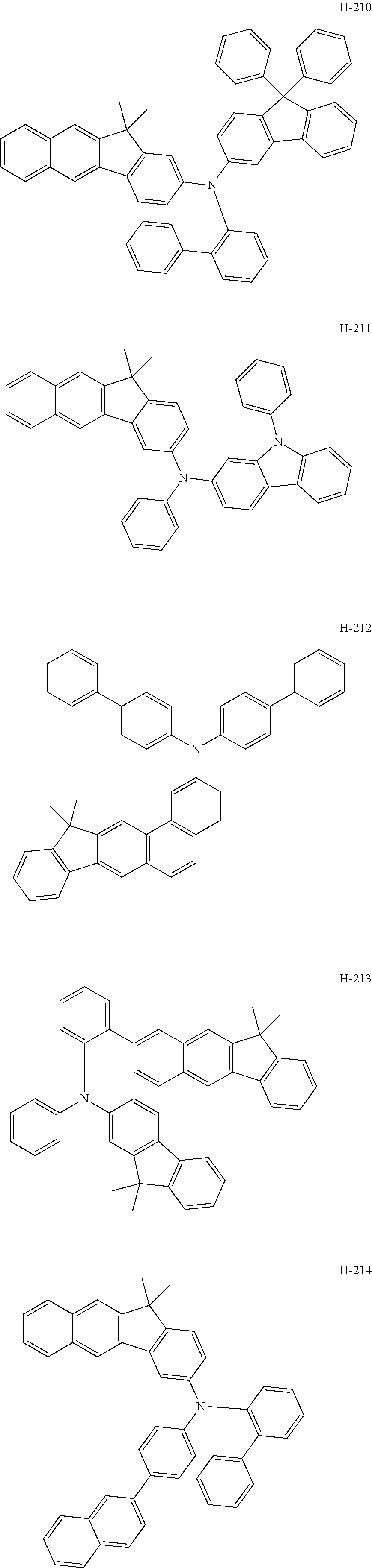 [SMILES: CC1(C)c2cc(N(c3ccc4c(c3)-c3ccccc3C4(c3ccccc3)c3ccccc3)c3ccccc3-c3ccccc3)ccc2-c2cc3ccccc3cc21 ; CC1(C)c2ccc(N(c3ccccc3)c3ccc4c5ccccc5n(-c5ccccc5)c4c3)cc2-c2cc3ccccc3cc21 ; CC1(C)c2ccccc2-c2cc3ccc4ccc(N(c5ccc(-c6ccccc6)cc5)c5ccc(-c6ccccc6)cc5)cc4c3cc21 ; CC1(C)c2ccccc2-c2ccc(N(c3ccccc3)c3ccccc3-c3ccc4cc5c(cc4c3)C(C)(C)c3ccccc3-5)cc21 ; CC1(C)c2ccc(N(c3ccc(-c4ccc5ccccc5c4)cc3)c3ccccc3-c3ccccc3)cc2-c2cc3ccccc3cc21]

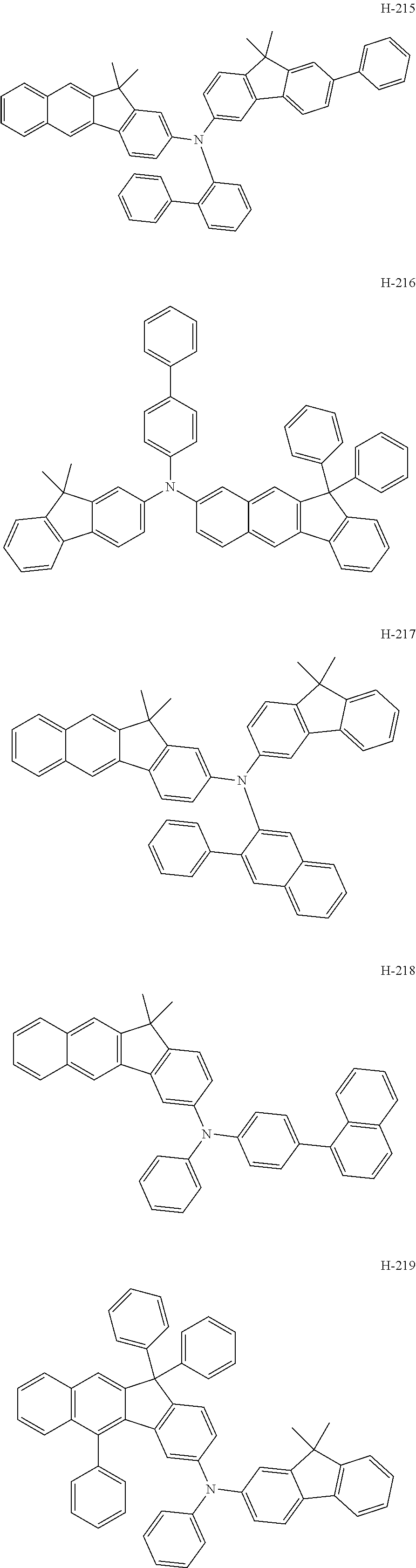 [SMILES: CC1(C)c2ccc(N(c3ccc4c(c3)C(C)(C)c3cc5ccccc5cc3-4)c3ccccc3-c3ccccc3)cc2-c2ccc(-c3ccccc3)cc21 ; CC1(C)c2ccccc2-c2ccc(N(c3ccc(-c4ccccc4)cc3)c3ccc4cc5c(cc4c3)C(c3ccccc3)(c3ccccc3)c3ccccc3-5)cc21 ; CC1(C)c2ccccc2-c2cc(N(c3ccc4c(c3)C(C)(C)c3cc5ccccc5cc3-4)c3cc4ccccc4cc3-c3ccccc3)ccc21 ; CC1(C)c2ccc(N(c3ccccc3)c3ccc(-c4cccc5ccccc45)cc3)cc2-c2cc3ccccc3cc21 ; CC1(C)c2ccccc2-c2ccc(N(c3ccccc3)c3ccc4c(c3)-c3c(cc5ccccc5c3-c3ccccc3)C4(c3ccccc3)c3ccccc3)cc21]

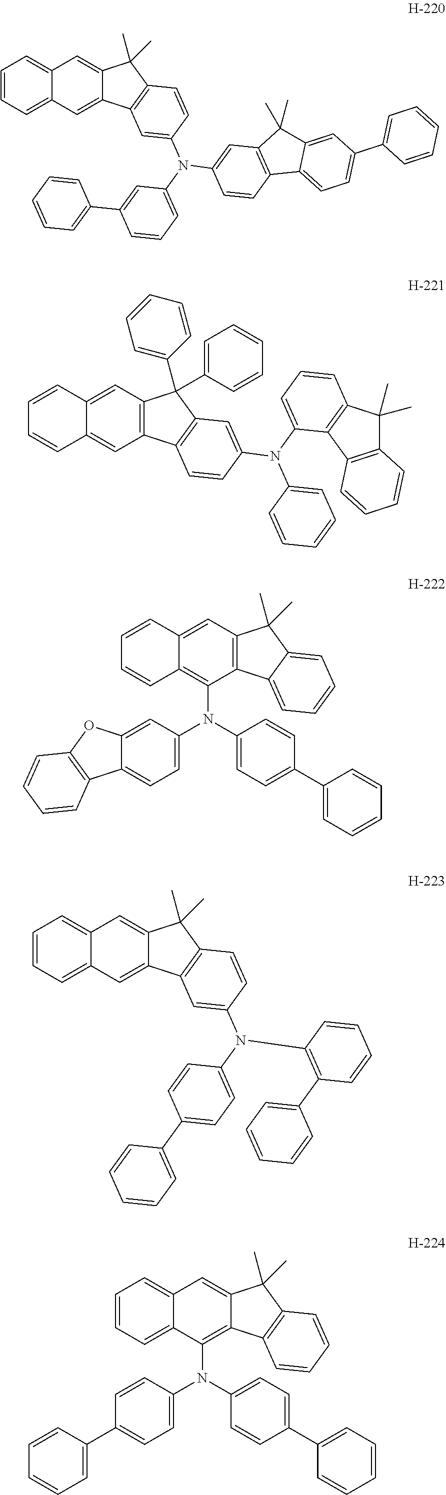 [SMILES: CC1(C)c2cc(-c3ccccc3)ccc2-c2ccc(N(c3cccc(-c4ccccc4)c3)c3ccc4c(c3)-c3cc5ccccc5cc3C4(C)C)cc21 ; CC1(C)c2ccccc2-c2c(N(c3ccccc3)c3ccc4c(c3)C(c3ccccc3)(c3ccccc3)c3cc5ccccc5cc3-4)cccc21 ; CC1(C)c2ccccc2-c2c1cc1ccccc1c2N(c1ccc(-c2ccccc2)cc1)c1ccc2c(c1)oc1ccccc12 ; CC1(C)c2ccc(N(c3ccc(-c4ccccc4)cc3)c3ccccc3-c3ccccc3)cc2-c2cc3ccccc3cc21 ; CC1(C)c2ccccc2-c2c1cc1ccccc1c2N(c1ccc(-c2ccccc2)cc1)c1ccc(-c2ccccc2)cc1]

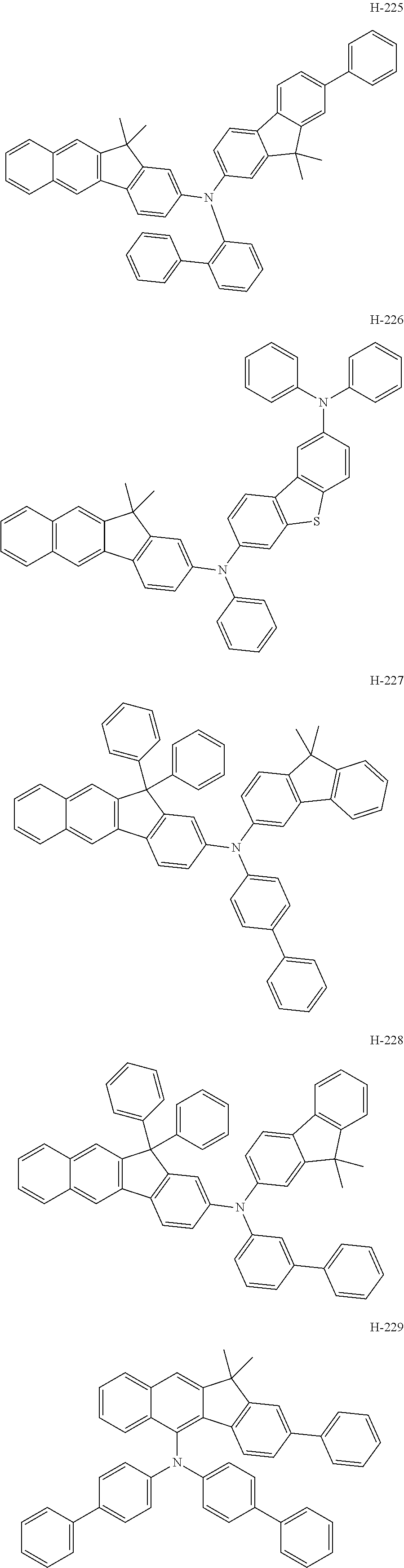 [SMILES: CC1(C)c2cc(-c3ccccc3)ccc2-c2ccc(N(c3ccc4c(c3)C(C)(C)c3cc5ccccc5cc3-4)c3ccccc3-c3ccccc3)cc21 ; CC1(C)c2cc(N(c3ccccc3)c3ccc4c(c3)sc3ccc(N(c5ccccc5)c5ccccc5)cc34)ccc2-c2cc3ccccc3cc21 ; CC1(C)c2ccccc2-c2cc(N(c3ccc(-c4ccccc4)cc3)c3ccc4c(c3)C(c3ccccc3)(c3ccccc3)c3cc5ccccc5cc3-4)ccc21 ; CC1(C)c2ccccc2-c2ccc(N(c3cccc(-c4ccccc4)c3)c3ccc4c(c3)C(c3ccccc3)(c3ccccc3)c3cc5ccccc5cc3-4)cc21 ; CC1(C)c2cc(-c3ccccc3)ccc2-c2c1cc1ccccc1c2N(c1ccc(-c2ccccc2)cc1)c1ccc(-c2ccccc2)cc1]

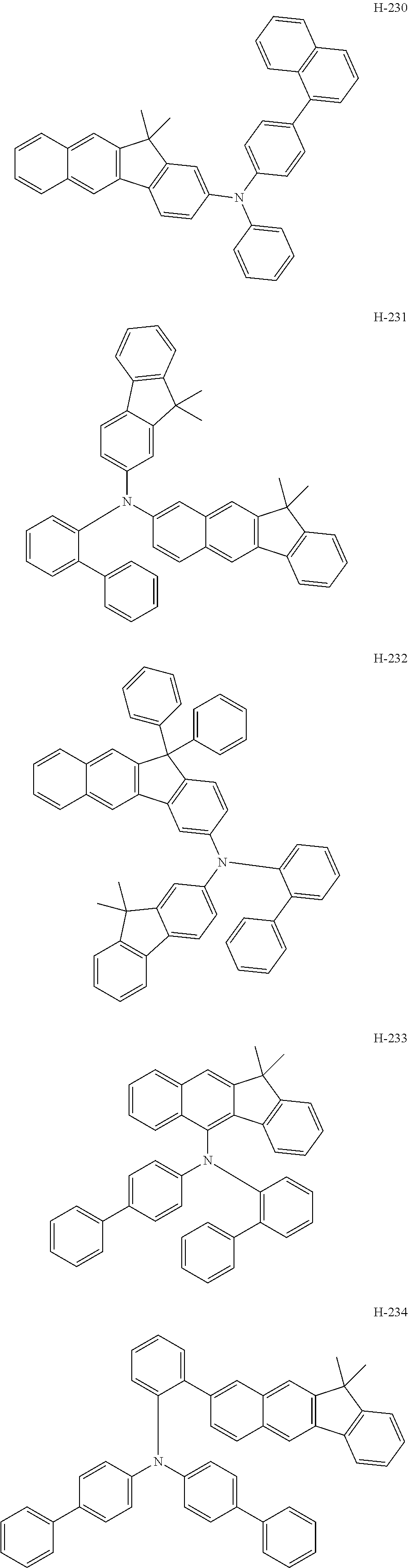 [SMILES: CC1(C)c2cc(N(c3ccccc3)c3ccc(-c4cccc5ccccc45)cc3)ccc2-c2cc3ccccc3cc21 ; CC1(C)c2ccccc2-c2ccc(N(c3ccc4cc5c(cc4c3)C(C)(C)c3ccccc3-5)c3ccccc3-c3ccccc3)cc21 ; CC1(C)c2ccccc2-c2ccc(N(c3ccc4c(c3)-c3cc5ccccc5cc3C4(c3ccccc3)c3ccccc3)c3ccccc3-c3ccccc3)cc21 ; CC1(C)c2ccccc2-c2c1cc1ccccc1c2N(c1ccc(-c2ccccc2)cc1)c1ccccc1-c1ccccc1 ; CC1(C)c2ccccc2-c2cc3ccc(-c4ccccc4N(c4ccc(-c5ccccc5)cc4)c4ccc(-c5ccccc5)cc4)cc3cc21]

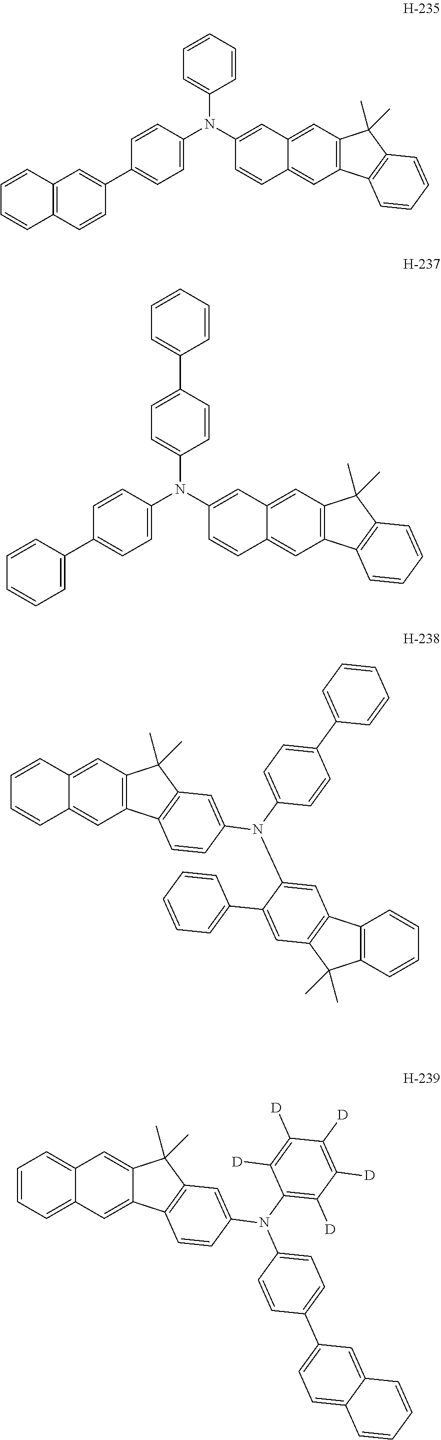 [SMILES: CC1(C)c2ccccc2-c2cc3ccc(N(c4ccccc4)c4ccc(-c5ccc6ccccc6c5)cc4)cc3cc21 ; CC1(C)c2ccccc2-c2cc3ccc(N(c4ccc(-c5ccccc5)cc4)c4ccc(-c5ccccc5)cc4)cc3cc21 ; CC1(C)c2ccccc2-c2cc(N(c3ccc(-c4ccccc4)cc3)c3ccc4c(c3)C(C)(C)c3cc5ccccc5cc3-4)c(-c3ccccc3)cc21 ; [2H]c1c([2H])c([2H])c(N(c2ccc(-c3ccc4ccccc4c3)cc2)c2ccc3c(c2)C(C)(C)c2cc4ccccc4cc2-3)c([2H])c1[2H]]

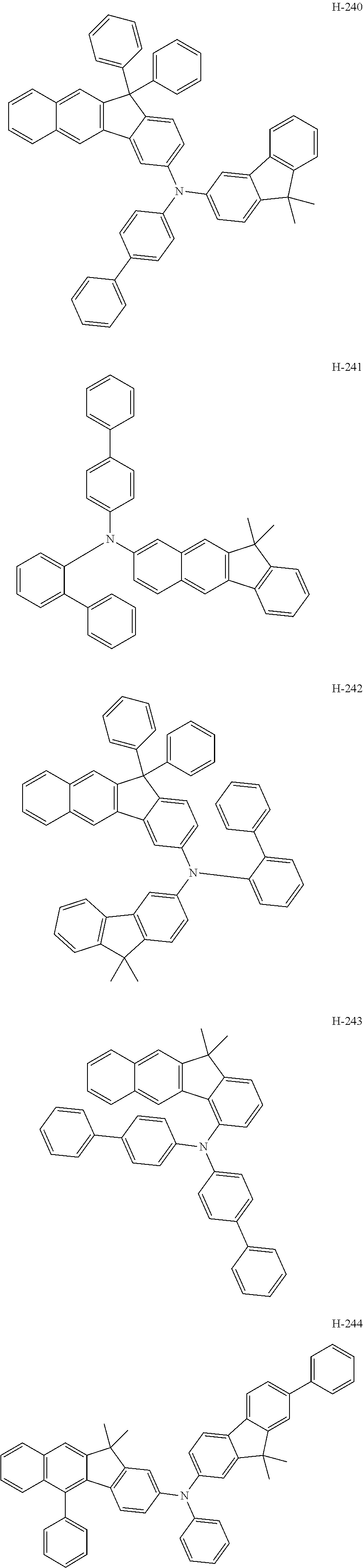 [SMILES: CC1(C)c2ccccc2-c2cc(N(c3ccc(-c4ccccc4)cc3)c3ccc4c(c3)-c3cc5ccccc5cc3C4(c3ccccc3)c3ccccc3)ccc21 ; CC1(C)c2ccccc2-c2cc3ccc(N(c4ccc(-c5ccccc5)cc4)c4ccccc4-c4ccccc4)cc3cc21 ; CC1(C)c2ccccc2-c2cc(N(c3ccc4c(c3)-c3cc5ccccc5cc3C4(c3ccccc3)c3ccccc3)c3ccccc3-c3ccccc3)ccc21 ; CC1(C)c2cc3ccccc3cc2-c2c(N(c3ccc(-c4ccccc4)cc3)c3ccc(-c4ccccc4)cc3)cccc21 ; CC1(C)c2cc(-c3ccccc3)ccc2-c2ccc(N(c3ccccc3)c3ccc4c(c3)C(C)(C)c3cc5ccccc5c(-c5ccccc5)c3-4)cc21]

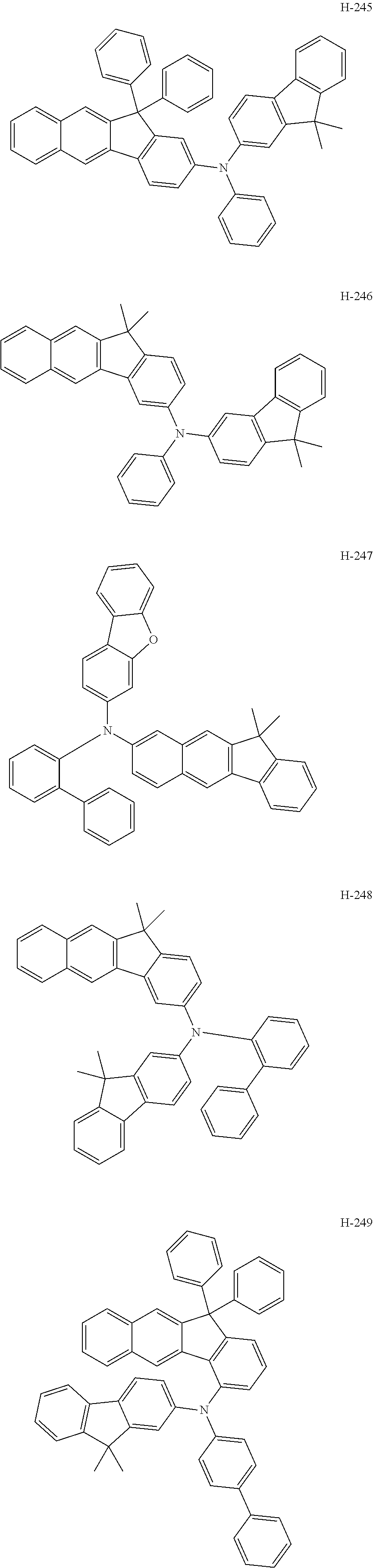 [SMILES: CC1(C)c2ccccc2-c2ccc(N(c3ccccc3)c3ccc4c(c3)C(c3ccccc3)(c3ccccc3)c3cc5ccccc5cc3-4)cc21 ; CC1(C)c2ccccc2-c2cc(N(c3ccccc3)c3ccc4c(c3)-c3cc5ccccc5cc3C4(C)C)ccc21 ; CC1(C)c2ccccc2-c2cc3ccc(N(c4ccc5c(c4)oc4ccccc45)c4ccccc4-c4ccccc4)cc3cc21 ; CC1(C)c2ccccc2-c2ccc(N(c3ccc4c(c3)-c3cc5ccccc5cc3C4(C)C)c3ccccc3-c3ccccc3)cc21 ; CC1(C)c2ccccc2-c2ccc(N(c3ccc(-c4ccccc4)cc3)c3cccc4c3-c3cc5ccccc5cc3C4(c3ccccc3)c3ccccc3)cc21]

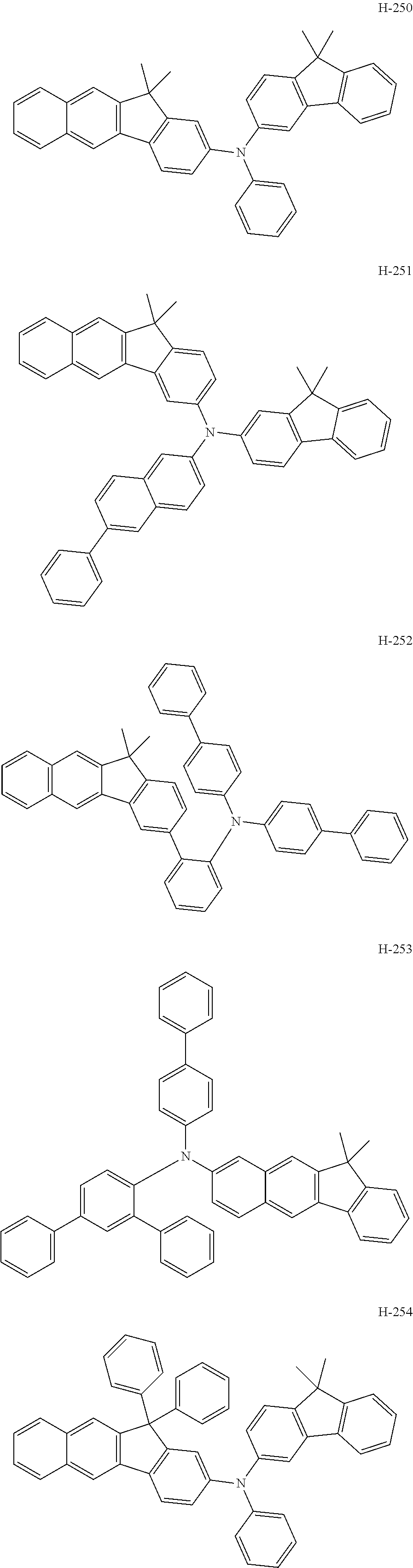 [SMILES: CC1(C)c2ccccc2-c2cc(N(c3ccccc3)c3ccc4c(c3)C(C)(C)c3cc5ccccc5cc3-4)ccc21 ; CC1(C)c2ccccc2-c2ccc(N(c3ccc4c(c3)-c3cc5ccccc5cc3C4(C)C)c3ccc4cc(-c5ccccc5)ccc4c3)cc21 ; CC1(C)c2ccc(-c3ccccc3N(c3ccc(-c4ccccc4)cc3)c3ccc(-c4ccccc4)cc3)cc2-c2cc3ccccc3cc21 ; CC1(C)c2ccccc2-c2cc3ccc(N(c4ccc(-c5ccccc5)cc4)c4ccc(-c5ccccc5)cc4-c4ccccc4)cc3cc21 ; CC1(C)c2ccccc2-c2cc(N(c3ccccc3)c3ccc4c(c3)C(c3ccccc3)(c3ccccc3)c3cc5ccccc5cc3-4)ccc21]

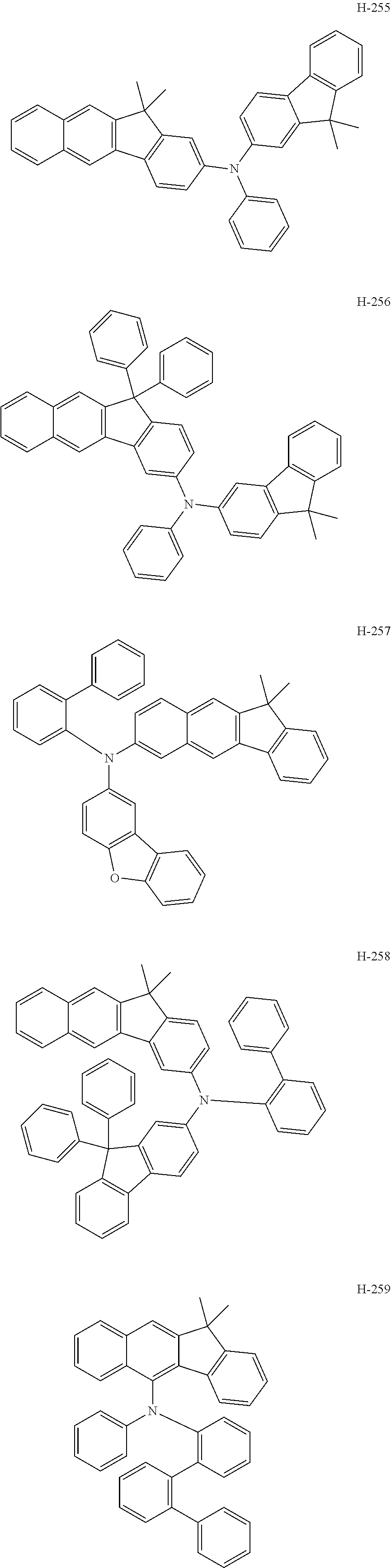 [SMILES: CC1(C)c2ccccc2-c2ccc(N(c3ccccc3)c3ccc4c(c3)C(C)(C)c3cc5ccccc5cc3-4)cc21 ; CC1(C)c2ccccc2-c2cc(N(c3ccccc3)c3ccc4c(c3)-c3cc5ccccc5cc3C4(c3ccccc3)c3ccccc3)ccc21 ; CC1(C)c2ccccc2-c2cc3cc(N(c4ccc5oc6ccccc6c5c4)c4ccccc4-c4ccccc4)ccc3cc21 ; CC1(C)c2ccc(N(c3ccc4c(c3)C(c3ccccc3)(c3ccccc3)c3ccccc3-4)c3ccccc3-c3ccccc3)cc2-c2cc3ccccc3cc21 ; CC1(C)c2ccccc2-c2c1cc1ccccc1c2N(c1ccccc1)c1ccccc1-c1ccccc1-c1ccccc1]

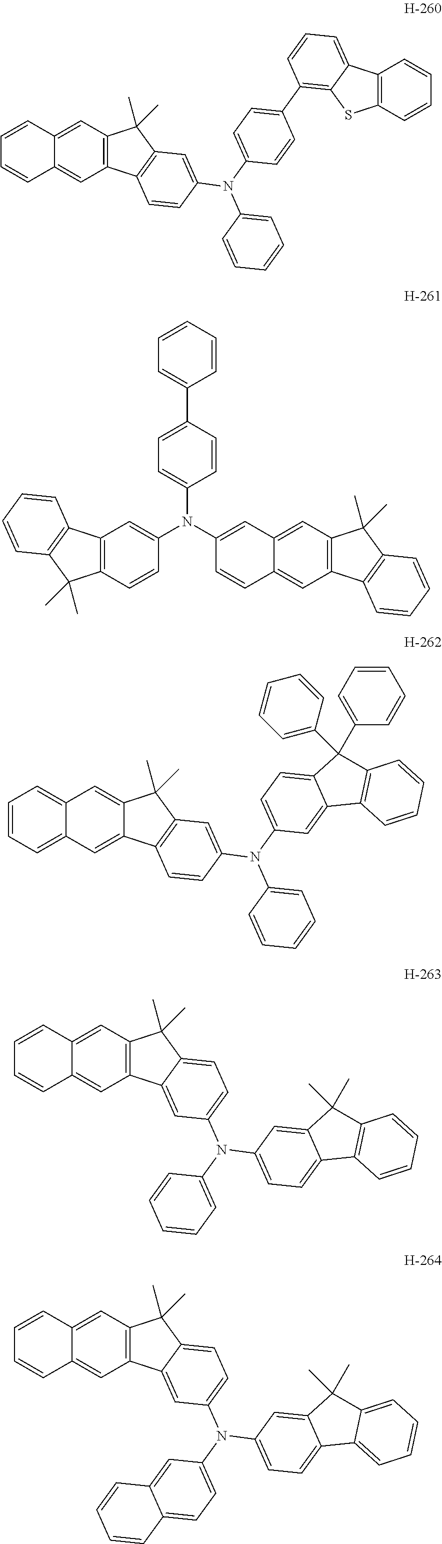 [SMILES: CC1(C)c2cc(N(c3ccccc3)c3ccc(-c4cccc5c4sc4ccccc45)cc3)ccc2-c2cc3ccccc3cc21 ; CC1(C)c2ccccc2-c2cc(N(c3ccc(-c4ccccc4)cc3)c3ccc4cc5c(cc4c3)C(C)(C)c3ccccc3-5)ccc21 ; CC1(C)c2cc(N(c3ccccc3)c3ccc4c(c3)-c3ccccc3C4(c3ccccc3)c3ccccc3)ccc2-c2cc3ccccc3cc21 ; CC1(C)c2ccccc2-c2ccc(N(c3ccccc3)c3ccc4c(c3)-c3cc5ccccc5cc3C4(C)C)cc21 ; CC1(C)c2ccccc2-c2ccc(N(c3ccc4c(c3)-c3cc5ccccc5cc3C4(C)C)c3ccc4ccccc4c3)cc21]

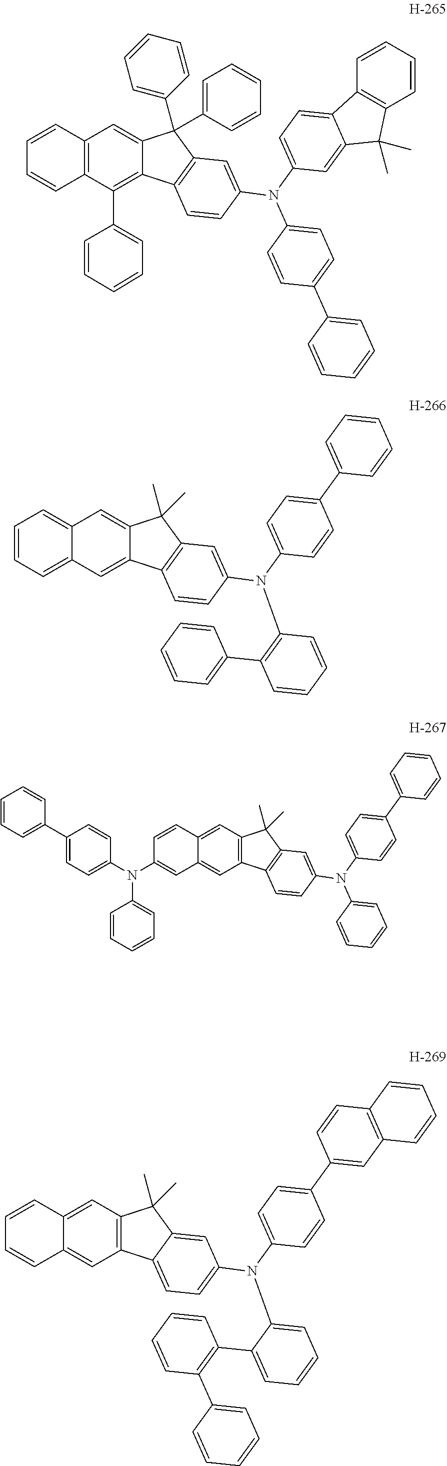

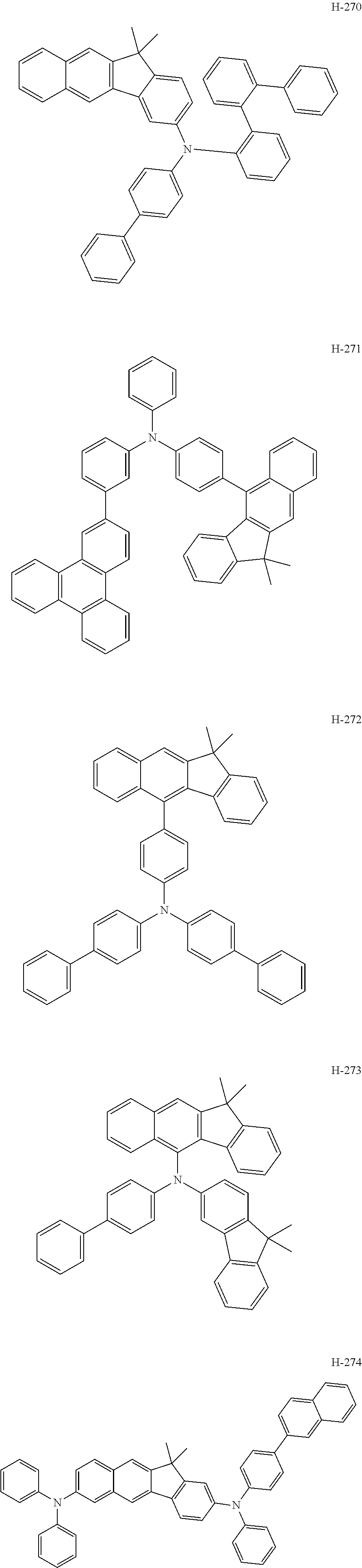 [SMILES: CC1(C)c2ccc(N(c3ccc(-c4ccccc4)cc3)c3ccccc3-c3ccccc3-c3ccccc3)cc2-c2cc3ccccc3cc21 ; CC1(C)c2ccccc2-c2c1cc1ccccc1c2-c1ccc(N(c2ccccc2)c2cccc(-c3ccc4c5ccccc5c5ccccc5c4c3)c2)cc1 ; CC1(C)c2ccccc2-c2c1cc1ccccc1c2-c1ccc(N(c2ccc(-c3ccccc3)cc2)c2ccc(-c3ccccc3)cc2)cc1 ; CC1(C)c2ccccc2-c2cc(N(c3ccc(-c4ccccc4)cc3)c3c4c(cc5ccccc35)C(C)(C)c3ccccc3-4)ccc21 ; CC1(C)c2cc(N(c3ccccc3)c3ccc(-c4ccc5ccccc5c4)cc3)ccc2-c2cc3cc(N(c4ccccc4)c4ccccc4)ccc3cc21]

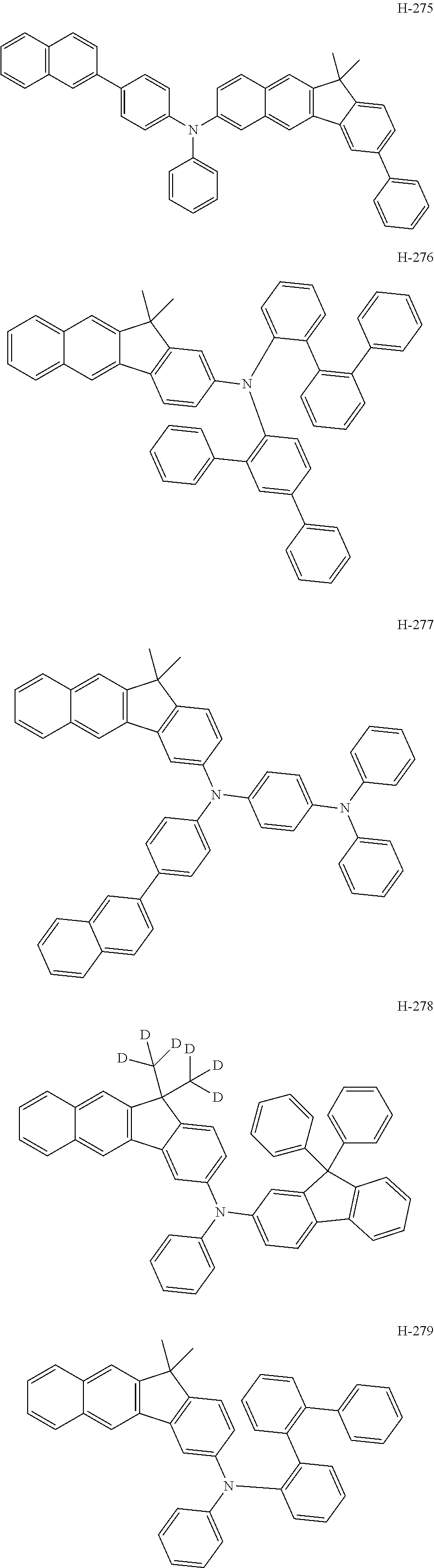 [SMILES: CC1(C)c2ccc(-c3ccccc3)cc2-c2cc3cc(N(c4ccccc4)c4ccc(-c5ccc6ccccc6c5)cc4)ccc3cc21 ; CC1(C)c2cc(N(c3ccc(-c4ccccc4)cc3-c3ccccc3)c3ccccc3-c3ccccc3-c3ccccc3)ccc2-c2cc3ccccc3cc21 ; CC1(C)c2ccc(N(c3ccc(-c4ccc5ccccc5c4)cc3)c3ccc(N(c4ccccc4)c4ccccc4)cc3)cc2-c2cc3ccccc3cc21 ; [2H]C([2H])([2H])C1(C([2H])([2H])[2H])c2ccc(N(c3ccccc3)c3ccc4c(c3)C(c3ccccc3)(c3ccccc3)c3ccccc3-4)cc2-c2cc3ccccc3cc21 ; CC1(C)c2ccc(N(c3ccccc3)c3ccccc3-c3ccccc3-c3ccccc3)cc2-c2cc3ccccc3cc21]

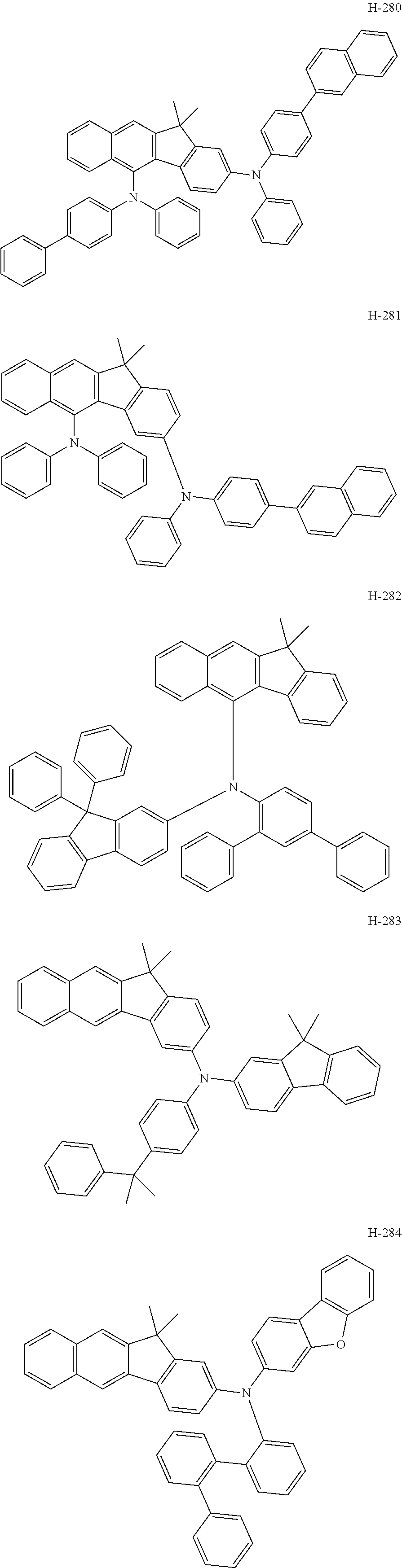 [SMILES: CC1(C)c2cc(N(c3ccccc3)c3ccc(-c4ccc5ccccc5c4)cc3)ccc2-c2c1cc1ccccc1c2N(c1ccccc1)c1ccc(-c2ccccc2)cc1 ; CC1(C)c2ccc(N(c3ccccc3)c3ccc(-c4ccc5ccccc5c4)cc3)cc2-c2c1cc1ccccc1c2N(c1ccccc1)c1ccccc1 ; CC1(C)c2ccccc2-c2c1cc1ccccc1c2N(c1ccc2c(c1)C(c1ccccc1)(c1ccccc1)c1ccccc1-2)c1ccc(-c2ccccc2)cc1-c1ccccc1 ; CC(C)(c1ccccc1)c1ccc(N(c2ccc3c(c2)-c2cc4ccccc4cc2C3(C)C)c2ccc3c(c2)C(C)(C)c2ccccc2-3)cc1 ; CC1(C)c2cc(N(c3ccc4c(c3)oc3ccccc34)c3ccccc3-c3ccccc3-c3ccccc3)ccc2-c2cc3ccccc3cc21]

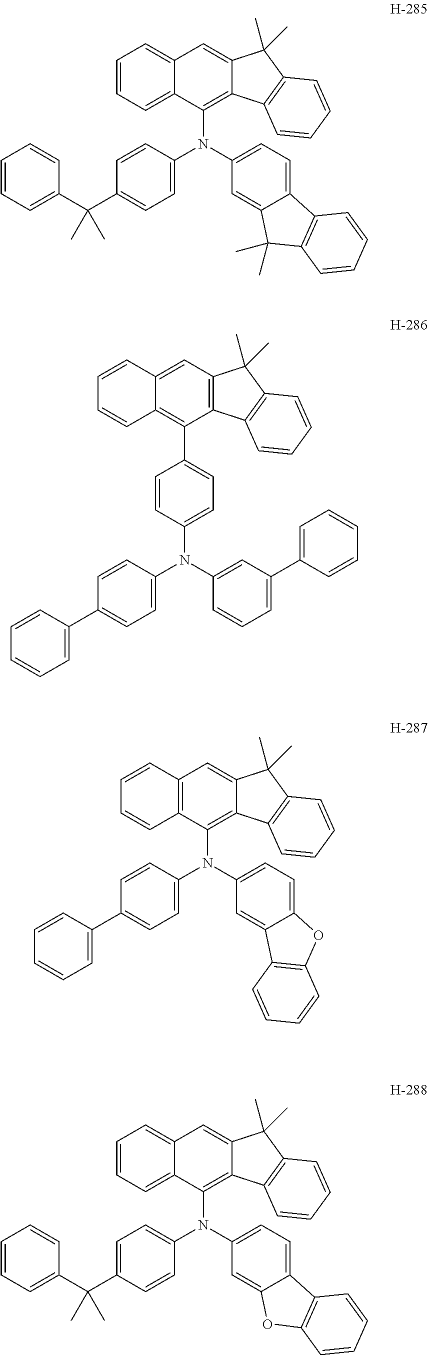 [SMILES: CC(C)(c1ccccc1)c1ccc(N(c2ccc3c(c2)C(C)(C)c2ccccc2-3)c2c3c(cc4ccccc24)C(C)(C)c2ccccc2-3)cc1 ; CC1(C)c2ccccc2-c2c1cc1ccccc1c2-c1ccc(N(c2ccc(-c3ccccc3)cc2)c2cccc(-c3ccccc3)c2)cc1 ; CC1(C)c2ccccc2-c2c1cc1ccccc1c2N(c1ccc(-c2ccccc2)cc1)c1ccc2oc3ccccc3c2c1 ; CC(C)(c1ccccc1)c1ccc(N(c2ccc3c(c2)oc2ccccc23)c2c3c(cc4ccccc24)C(C)(C)c2ccccc2-3)cc1]

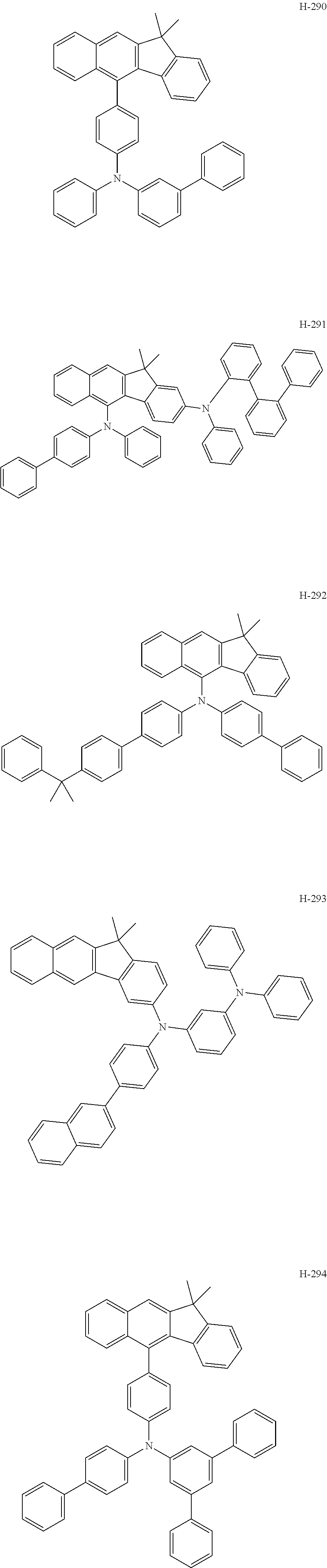 [SMILES: CC1(C)c2ccccc2-c2c1cc1ccccc1c2-c1ccc(N(c2ccccc2)c2cccc(-c3ccccc3)c2)cc1 ; CC1(C)c2cc(N(c3ccccc3)c3ccccc3-c3ccccc3-c3ccccc3)ccc2-c2c1cc1ccccc1c2N(c1ccccc1)c1ccc(-c2ccccc2)cc1 ; CC(C)(c1ccccc1)c1ccc(-c2ccc(N(c3ccc(-c4ccccc4)cc3)c3c4c(cc5ccccc35)C(C)(C)c3ccccc3-4)cc2)cc1 ; CC1(C)c2ccc(N(c3ccc(-c4ccc5ccccc5c4)cc3)c3cccc(N(c4ccccc4)c4ccccc4)c3)cc2-c2cc3ccccc3cc21 ; CC1(C)c2ccccc2-c2c1cc1ccccc1c2-c1ccc(N(c2ccc(-c3ccccc3)cc2)c2cc(-c3ccccc3)cc(-c3ccccc3)c2)cc1]

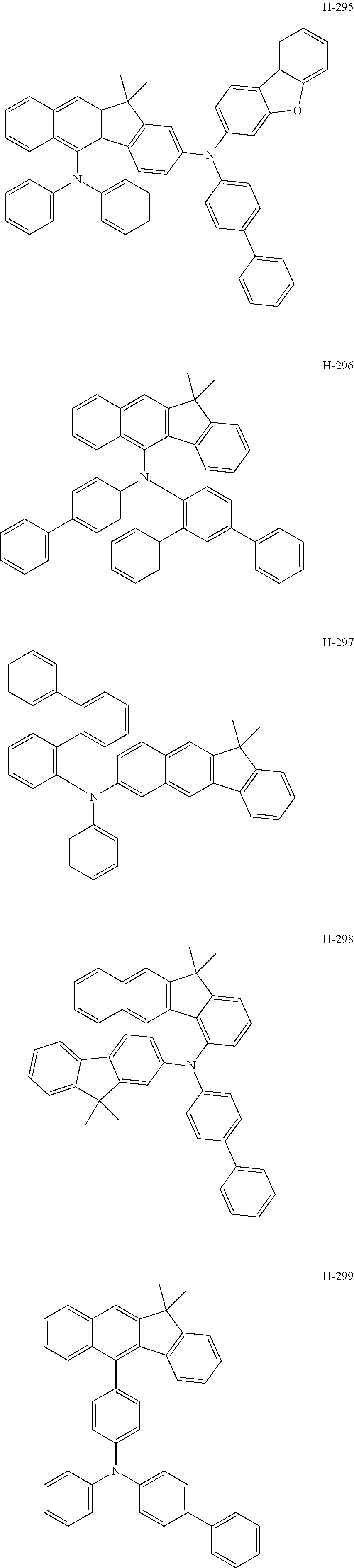 [SMILES: CC1(C)c2cc(N(c3ccc(-c4ccccc4)cc3)c3ccc4c(c3)oc3ccccc34)ccc2-c2c1cc1ccccc1c2N(c1ccccc1)c1ccccc1 ; CC1(C)c2ccccc2-c2c1cc1ccccc1c2N(c1ccc(-c2ccccc2)cc1)c1ccc(-c2ccccc2)cc1-c1ccccc1 ; CC1(C)c2ccccc2-c2cc3cc(N(c4ccccc4)c4ccccc4-c4ccccc4-c4ccccc4)ccc3cc21 ; CC1(C)c2ccccc2-c2ccc(N(c3ccc(-c4ccccc4)cc3)c3cccc4c3-c3cc5ccccc5cc3C4(C)C)cc21 ; CC1(C)c2ccccc2-c2c1cc1ccccc1c2-c1ccc(N(c2ccccc2)c2ccc(-c3ccccc3)cc2)cc1]

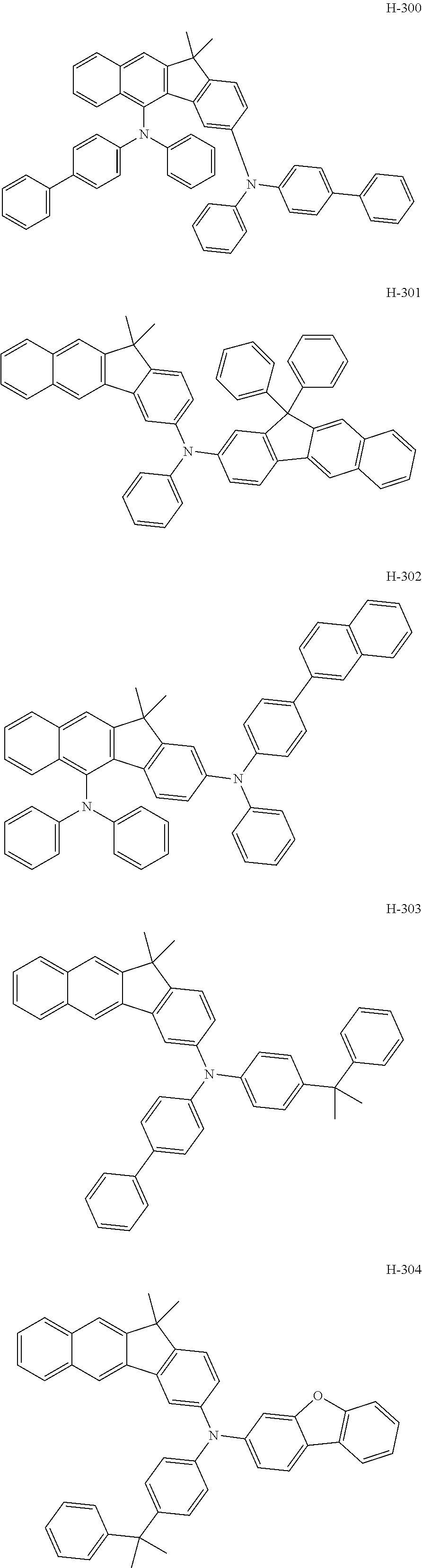 [SMILES: CC1(C)c2ccc(N(c3ccccc3)c3ccc(-c4ccccc4)cc3)cc2-c2c1cc1ccccc1c2N(c1ccccc1)c1ccc(-c2ccccc2)cc1 ; CC1(C)c2ccc(N(c3ccccc3)c3ccc4c(c3)C(c3ccccc3)(c3ccccc3)c3cc5ccccc5cc3-4)cc2-c2cc3ccccc3cc21 ; CC1(C)c2cc(N(c3ccccc3)c3ccc(-c4ccc5ccccc5c4)cc3)ccc2-c2c1cc1ccccc1c2N(c1ccccc1)c1ccccc1 ; CC(C)(c1ccccc1)c1ccc(N(c2ccc(-c3ccccc3)cc2)c2ccc3c(c2)-c2cc4ccccc4cc2C3(C)C)cc1 ; CC(C)(c1ccccc1)c1ccc(N(c2ccc3c(c2)-c2cc4ccccc4cc2C3(C)C)c2ccc3c(c2)oc2ccccc23)cc1]

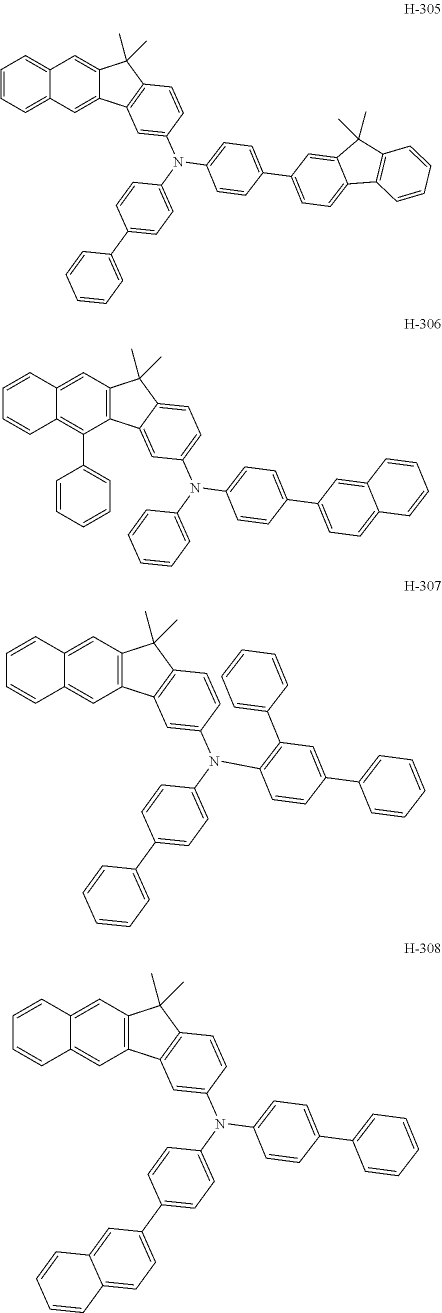 [SMILES: CC1(C)c2ccccc2-c2ccc(-c3ccc(N(c4ccc(-c5ccccc5)cc4)c4ccc5c(c4)-c4cc6ccccc6cc4C5(C)C)cc3)cc21 ; CC1(C)c2ccc(N(c3ccccc3)c3ccc(-c4ccc5ccccc5c4)cc3)cc2-c2c1cc1ccccc1c2-c1ccccc1 ; CC1(C)c2ccc(N(c3ccc(-c4ccccc4)cc3)c3ccc(-c4ccccc4)cc3-c3ccccc3)cc2-c2cc3ccccc3cc21 ; CC1(C)c2ccc(N(c3ccc(-c4ccccc4)cc3)c3ccc(-c4ccc5ccccc5c4)cc3)cc2-c2cc3ccccc3cc21]

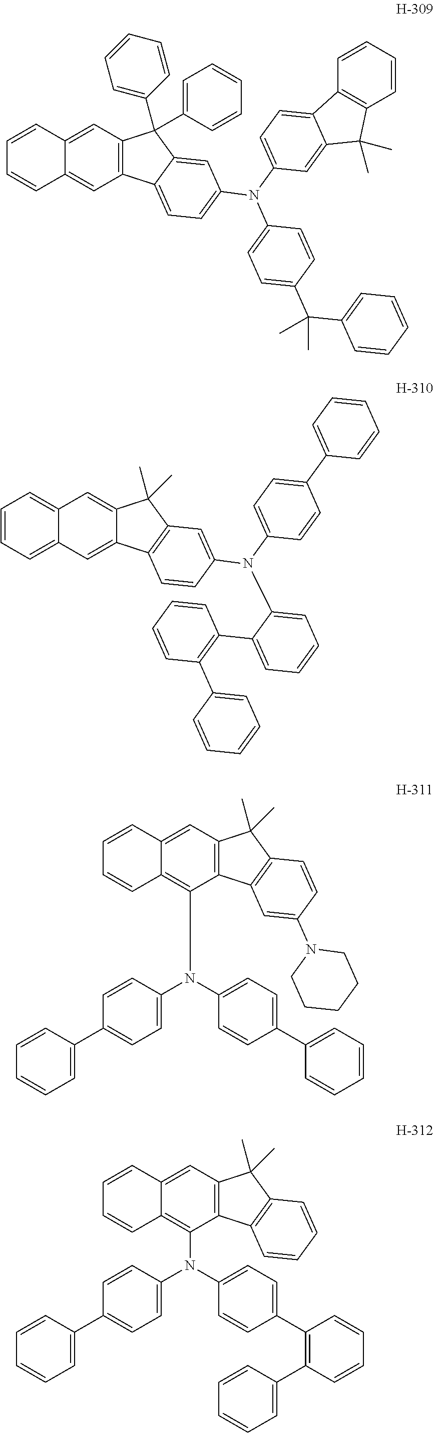

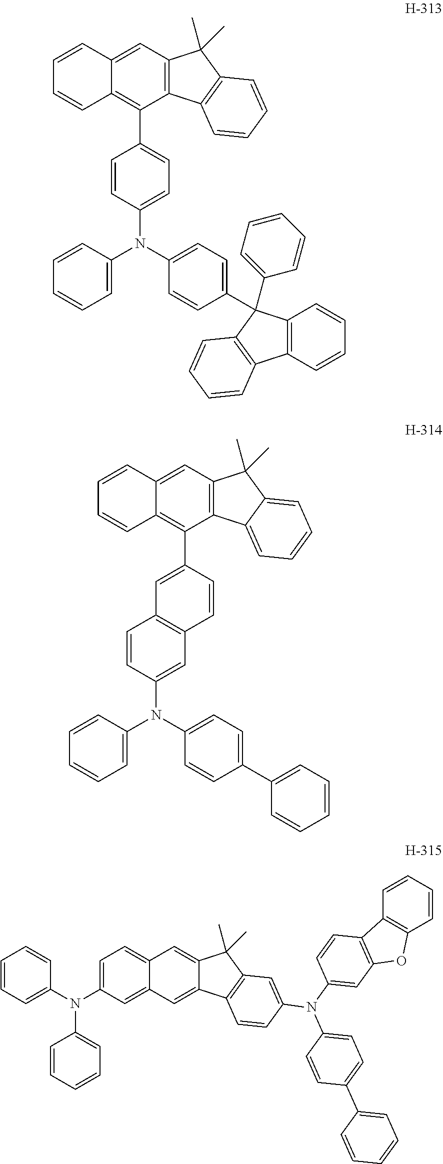

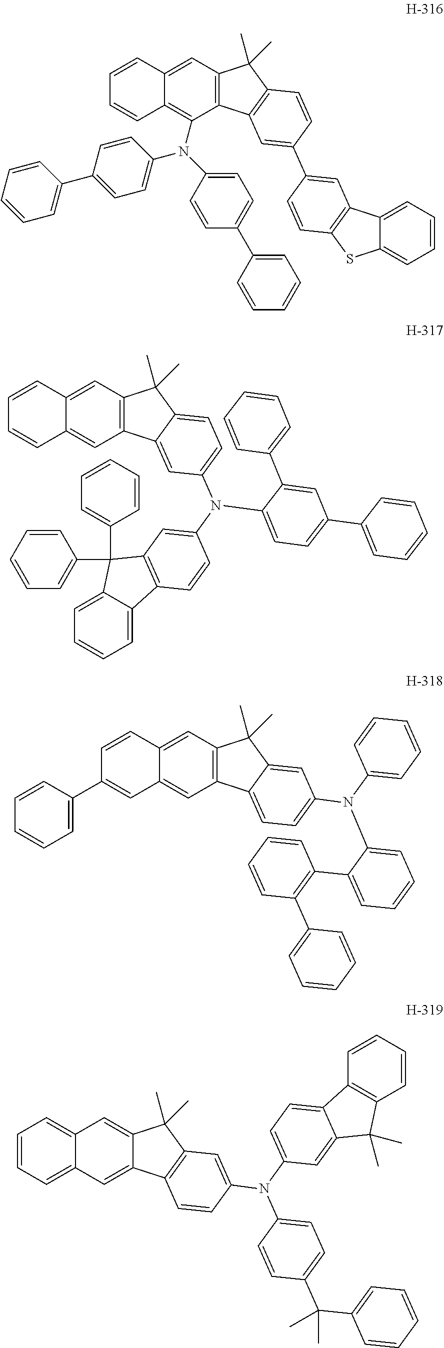 [SMILES: CC1(C)c2ccc(-c3ccc4sc5ccccc5c4c3)cc2-c2c1cc1ccccc1c2N(c1ccc(-c2ccccc2)cc1)c1ccc(-c2ccccc2)cc1 ; CC1(C)c2ccc(N(c3ccc4c(c3)C(c3ccccc3)(c3ccccc3)c3ccccc3-4)c3ccc(-c4ccccc4)cc3-c3ccccc3)cc2-c2cc3ccccc3cc21 ; CC1(C)c2cc(N(c3ccccc3)c3ccccc3-c3ccccc3-c3ccccc3)ccc2-c2cc3cc(-c4ccccc4)ccc3cc21 ; CC(C)(c1ccccc1)c1ccc(N(c2ccc3c(c2)C(C)(C)c2ccccc2-3)c2ccc3c(c2)C(C)(C)c2cc4ccccc4cc2-3)cc1]

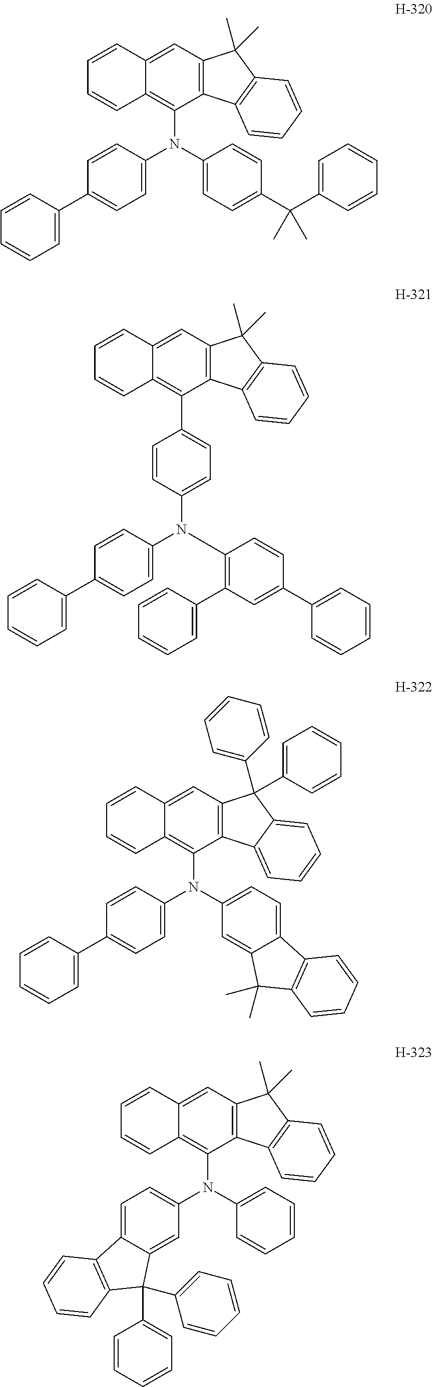

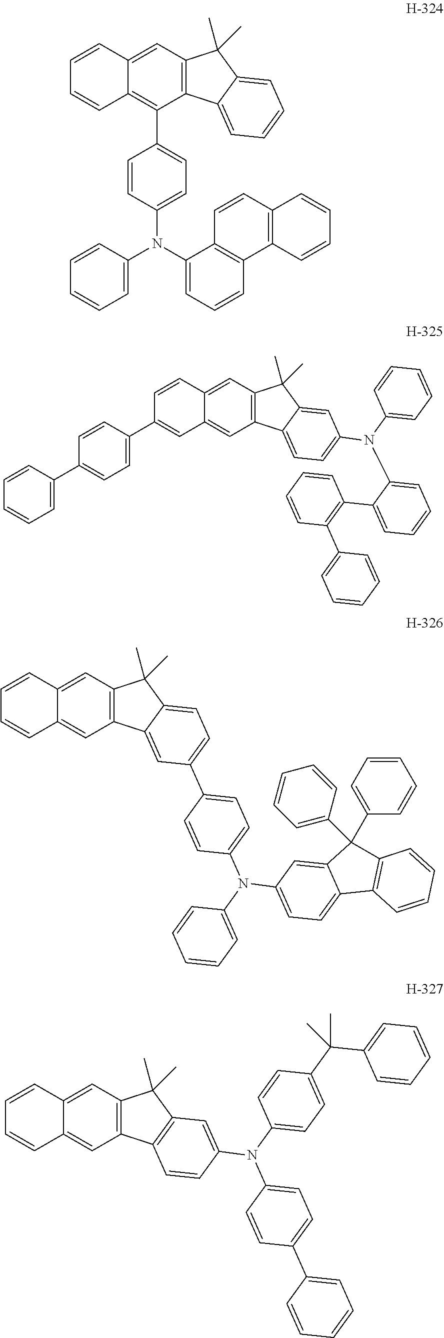 [SMILES: CC1(C)c2ccccc2-c2c1cc1ccccc1c2-c1ccc(N(c2ccccc2)c2cccc3c2ccc2ccccc23)cc1 ; CC1(C)c2cc(N(c3ccccc3)c3ccccc3-c3ccccc3-c3ccccc3)ccc2-c2cc3cc(-c4ccc(-c5ccccc5)cc4)ccc3cc21 ; CC1(C)c2ccc(-c3ccc(N(c4ccccc4)c4ccc5c(c4)C(c4ccccc4)(c4ccccc4)c4ccccc4-5)cc3)cc2-c2cc3ccccc3cc21 ; CC(C)(c1ccccc1)c1ccc(N(c2ccc(-c3ccccc3)cc2)c2ccc3c(c2)C(C)(C)c2cc4ccccc4cc2-3)cc1]

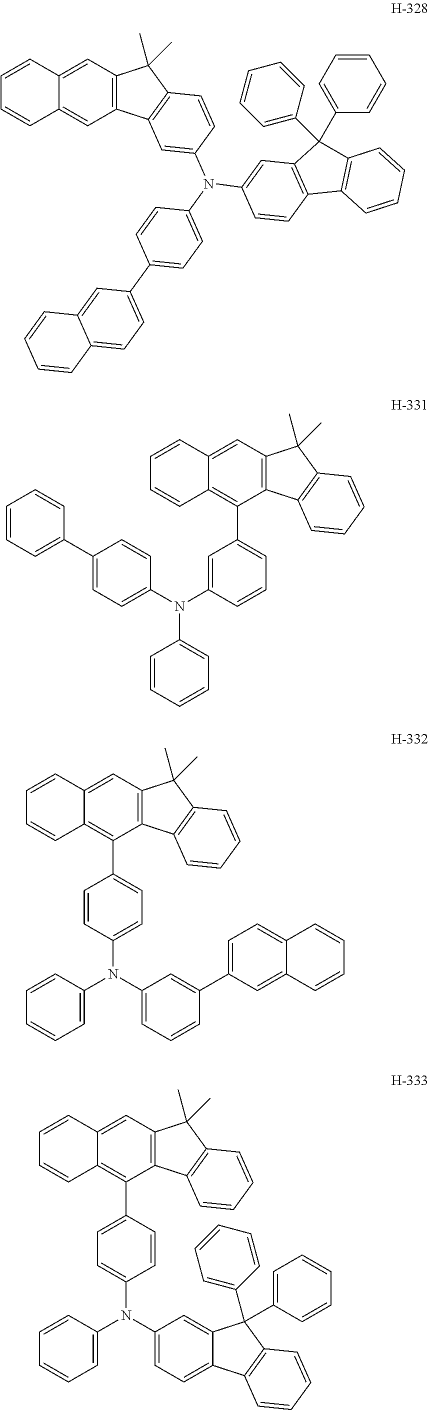

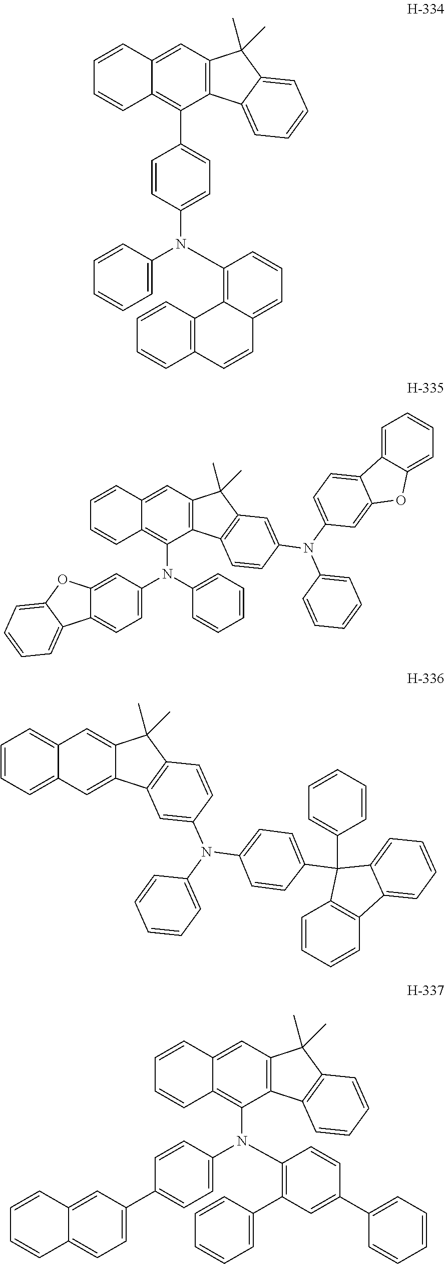 [SMILES: CC1(C)c2ccccc2-c2c1cc1ccccc1c2-c1ccc(N(c2ccccc2)c2cccc3ccc4ccccc4c23)cc1 ; CC1(C)c2cc(N(c3ccccc3)c3ccc4c(c3)oc3ccccc34)ccc2-c2c1cc1ccccc1c2N(c1ccccc1)c1ccc2c(c1)oc1ccccc12 ; CC1(C)c2ccc(N(c3ccccc3)c3ccc(C4(c5ccccc5)c5ccccc5-c5ccccc54)cc3)cc2-c2cc3ccccc3cc21 ; CC1(C)c2ccccc2-c2c1cc1ccccc1c2N(c1ccc(-c2ccc3ccccc3c2)cc1)c1ccc(-c2ccccc2)cc1-c1ccccc1]

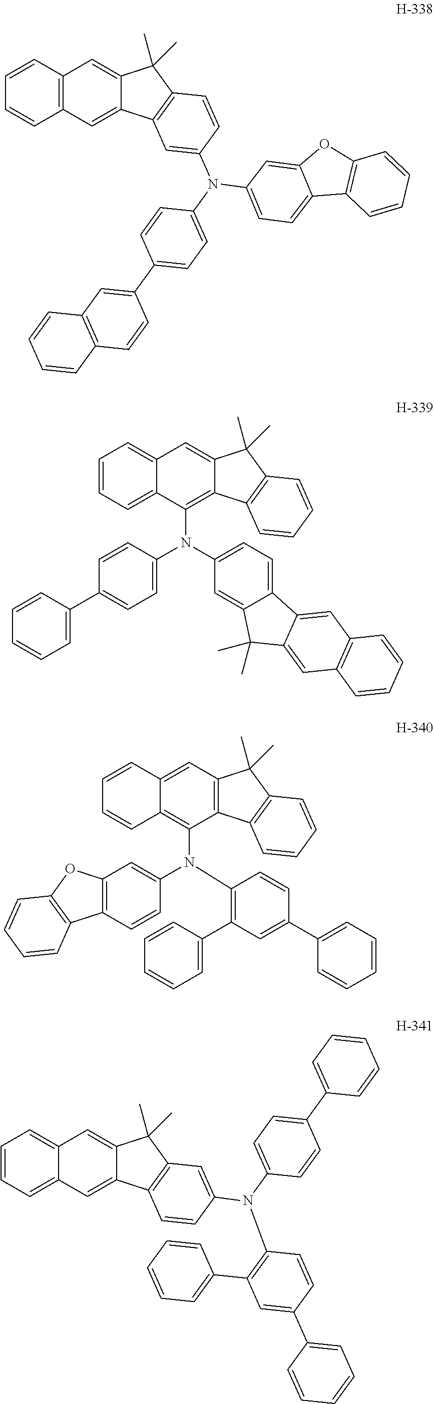 [SMILES: CC1(C)c2ccc(N(c3ccc(-c4ccc5ccccc5c4)cc3)c3ccc4c(c3)oc3ccccc34)cc2-c2cc3ccccc3cc21 ; CC1(C)c2cc(N(c3ccc(-c4ccccc4)cc3)c3c4c(cc5ccccc35)C(C)(C)c3ccccc3-4)ccc2-c2cc3ccccc3cc21 ; CC1(C)c2ccccc2-c2c1cc1ccccc1c2N(c1ccc2c(c1)oc1ccccc12)c1ccc(-c2ccccc2)cc1-c1ccccc1 ; CC1(C)c2cc(N(c3ccc(-c4ccccc4)cc3)c3ccc(-c4ccccc4)cc3-c3ccccc3)ccc2-c2cc3ccccc3cc21]

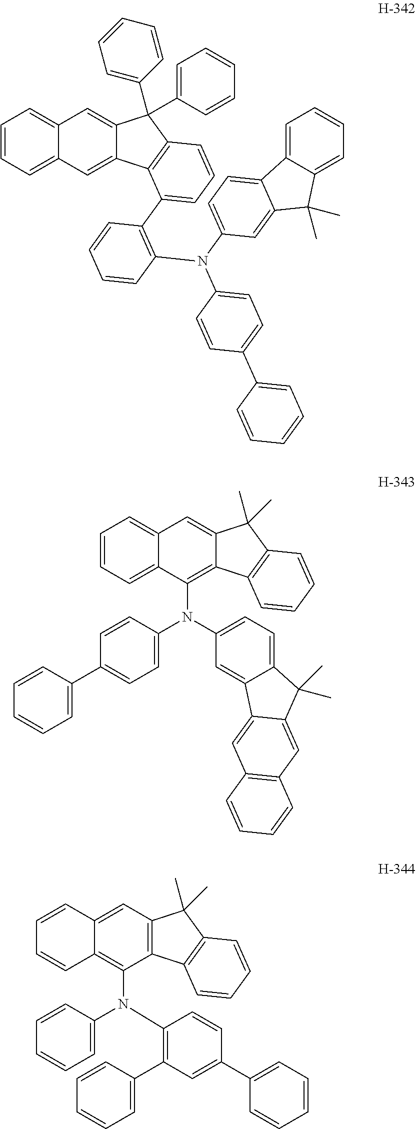 [SMILES: CC1(C)c2ccccc2-c2ccc(N(c3ccc(-c4ccccc4)cc3)c3ccccc3-c3cccc4c3-c3cc5ccccc5cc3C4(c3ccccc3)c3ccccc3)cc21 ; CC1(C)c2ccc(N(c3ccc(-c4ccccc4)cc3)c3c4c(cc5ccccc35)C(C)(C)c3ccccc3-4)cc2-c2cc3ccccc3cc21 ; CC1(C)c2ccccc2-c2c1cc1ccccc1c2N(c1ccccc1)c1ccc(-c2ccccc2)cc1-c1ccccc1]

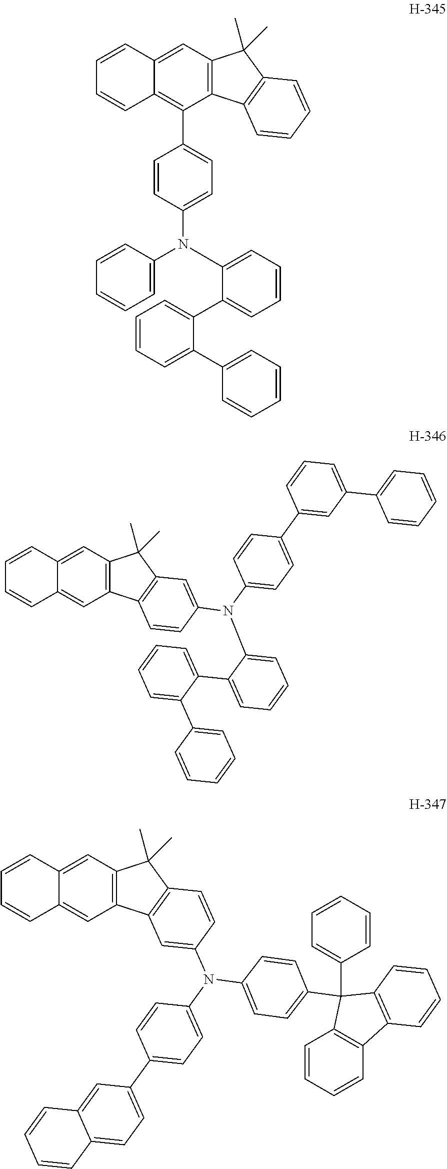 [SMILES: CC1(C)c2ccccc2-c2c1cc1ccccc1c2-c1ccc(N(c2ccccc2)c2ccccc2-c2ccccc2-c2ccccc2)cc1 ; CC1(C)c2cc(N(c3ccc(-c4cccc(-c5ccccc5)c4)cc3)c3ccccc3-c3ccccc3-c3ccccc3)ccc2-c2cc3ccccc3cc21 ; CC1(C)c2ccc(N(c3ccc(-c4ccc5ccccc5c4)cc3)c3ccc(C4(c5ccccc5)c5ccccc5-c5ccccc54)cc3)cc2-c2cc3ccccc3cc21]

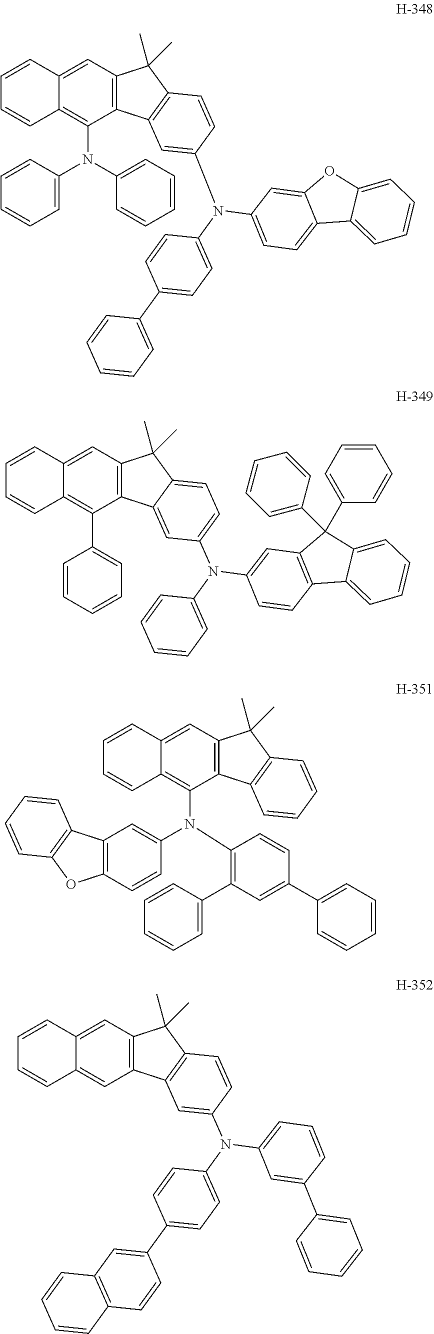

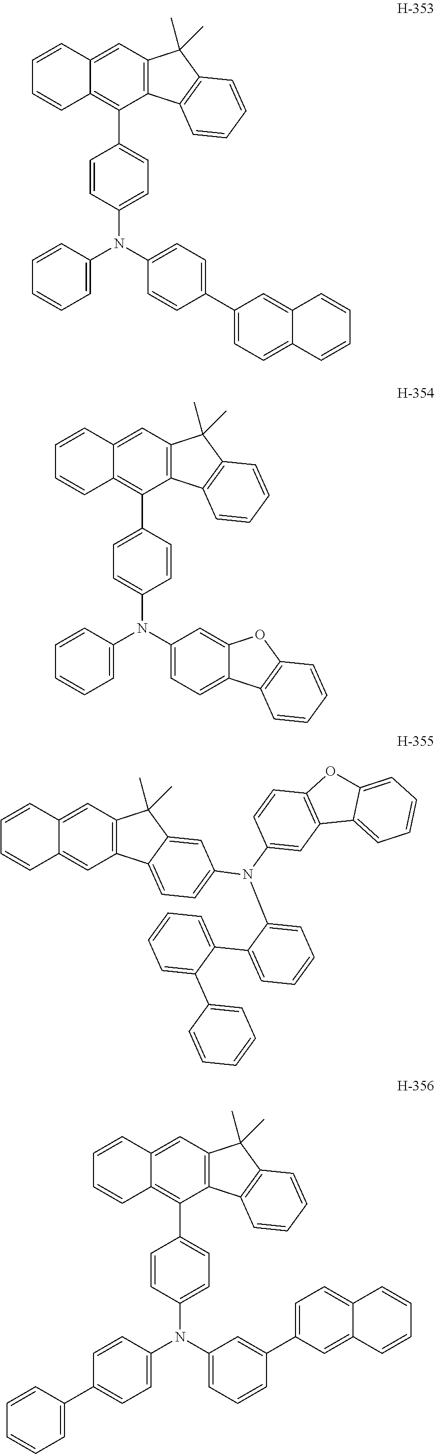

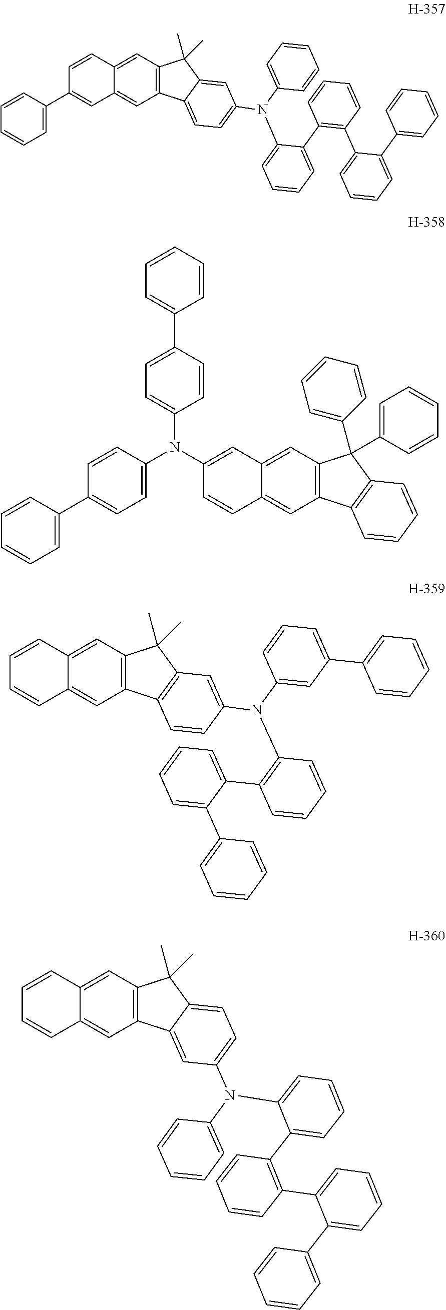 [SMILES: CC1(C)c2cc(N(c3ccccc3)c3ccccc3-c3ccccc3-c3ccccc3-c3ccccc3)ccc2-c2cc3cc(-c4ccccc4)ccc3cc21 ; c1ccc(-c2ccc(N(c3ccc(-c4ccccc4)cc3)c3ccc4cc5c(cc4c3)C(c3ccccc3)(c3ccccc3)c3ccccc3-5)cc2)cc1 ; CC1(C)c2cc(N(c3cccc(-c4ccccc4)c3)c3ccccc3-c3ccccc3-c3ccccc3)ccc2-c2cc3ccccc3cc21 ; CC1(C)c2ccc(N(c3ccccc3)c3ccccc3-c3ccccc3-c3ccccc3-c3ccccc3)cc2-c2cc3ccccc3cc21]

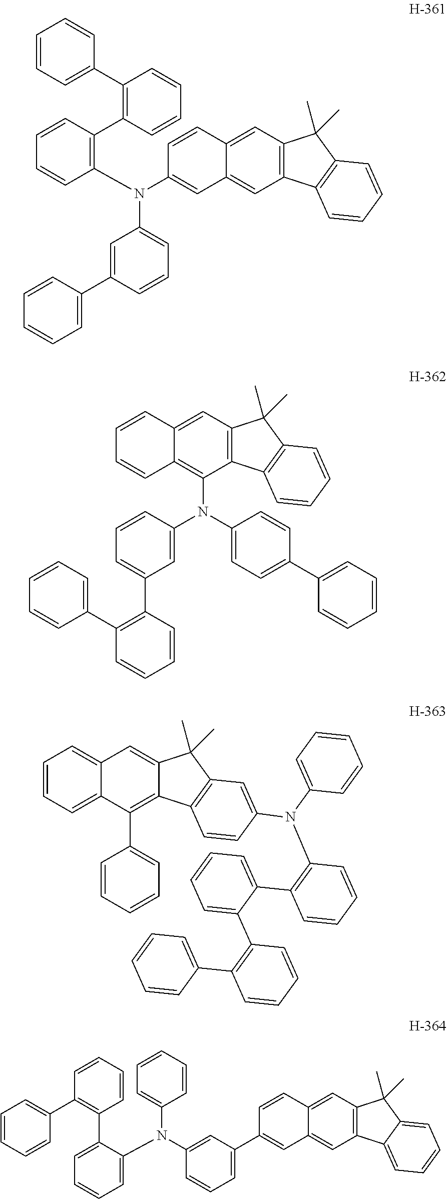 [SMILES: CC1(C)c2ccccc2-c2cc3cc(N(c4cccc(-c5ccccc5)c4)c4ccccc4-c4ccccc4-c4ccccc4)ccc3cc21 ; CC1(C)c2ccccc2-c2c1cc1ccccc1c2N(c1ccc(-c2ccccc2)cc1)c1cccc(-c2ccccc2-c2ccccc2)c1 ; CC1(C)c2cc(N(c3ccccc3)c3ccccc3-c3ccccc3-c3ccccc3-c3ccccc3)ccc2-c2c1cc1ccccc1c2-c1ccccc1 ; CC1(C)c2ccccc2-c2cc3cc(-c4cccc(N(c5ccccc5)c5ccccc5-c5ccccc5-c5ccccc5)c4)ccc3cc21]

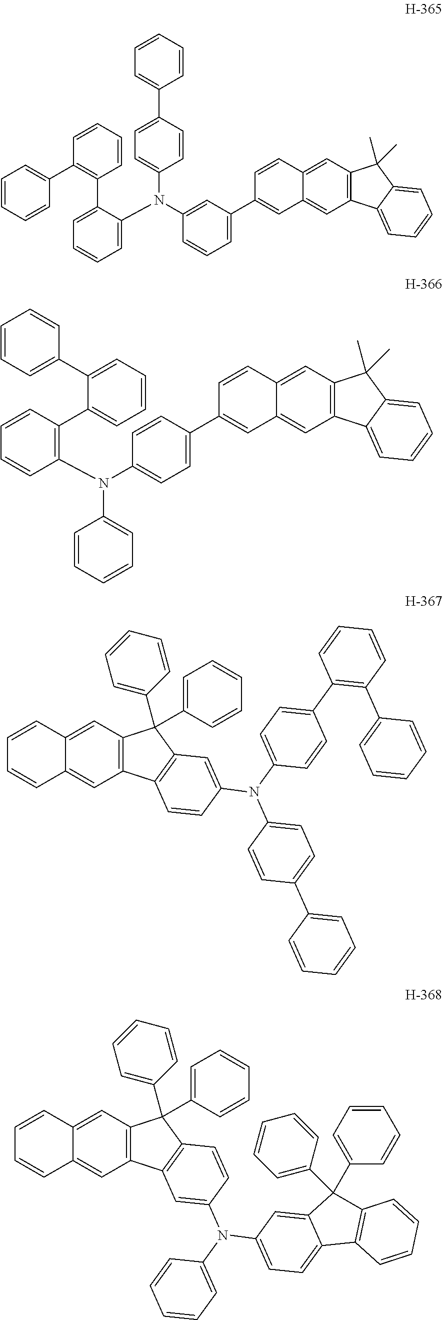 [SMILES: CC1(C)c2ccccc2-c2cc3cc(-c4cccc(N(c5ccc(-c6ccccc6)cc5)c5ccccc5-c5ccccc5-c5ccccc5)c4)ccc3cc21 ; CC1(C)c2ccccc2-c2cc3cc(-c4ccc(N(c5ccccc5)c5ccccc5-c5ccccc5-c5ccccc5)cc4)ccc3cc21 ; c1ccc(-c2ccc(N(c3ccc(-c4ccccc4-c4ccccc4)cc3)c3ccc4c(c3)C(c3ccccc3)(c3ccccc3)c3cc5ccccc5cc3-4)cc2)cc1 ; c1ccc(N(c2ccc3c(c2)-c2cc4ccccc4cc2C3(c2ccccc2)c2ccccc2)c2ccc3c(c2)C(c2ccccc2)(c2ccccc2)c2ccccc2-3)cc1]

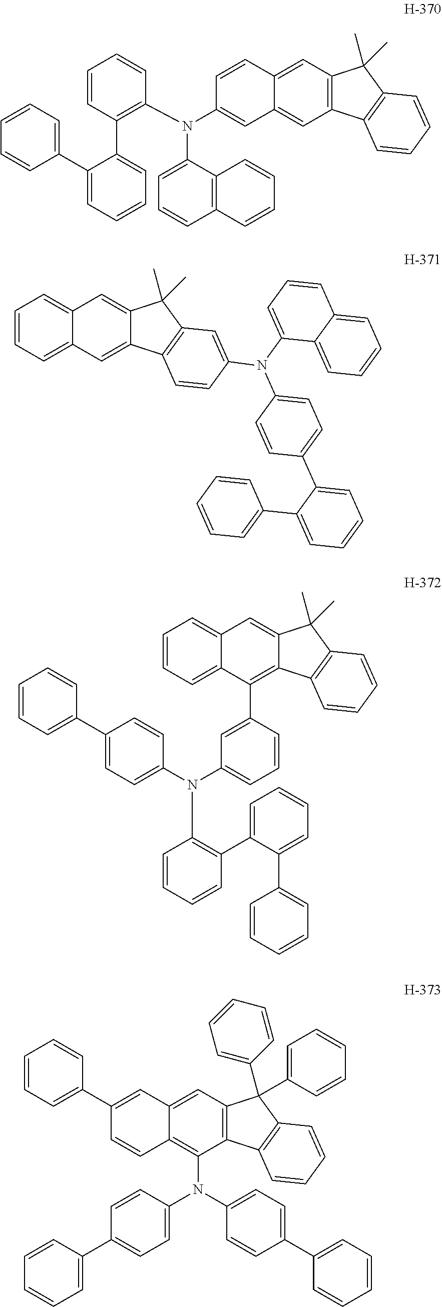 [SMILES: CC1(C)c2ccccc2-c2cc3cc(N(c4ccccc4-c4ccccc4-c4ccccc4)c4cccc5ccccc45)ccc3cc21 ; CC1(C)c2cc(N(c3ccc(-c4ccccc4-c4ccccc4)cc3)c3cccc4ccccc34)ccc2-c2cc3ccccc3cc21 ; CC1(C)c2ccccc2-c2c1cc1ccccc1c2-c1cccc(N(c2ccc(-c3ccccc3)cc2)c2ccccc2-c2ccccc2-c2ccccc2)c1 ; c1ccc(-c2ccc(N(c3ccc(-c4ccccc4)cc3)c3c4c(cc5cc(-c6ccccc6)ccc35)C(c3ccccc3)(c3ccccc3)c3ccccc3-4)cc2)cc1]

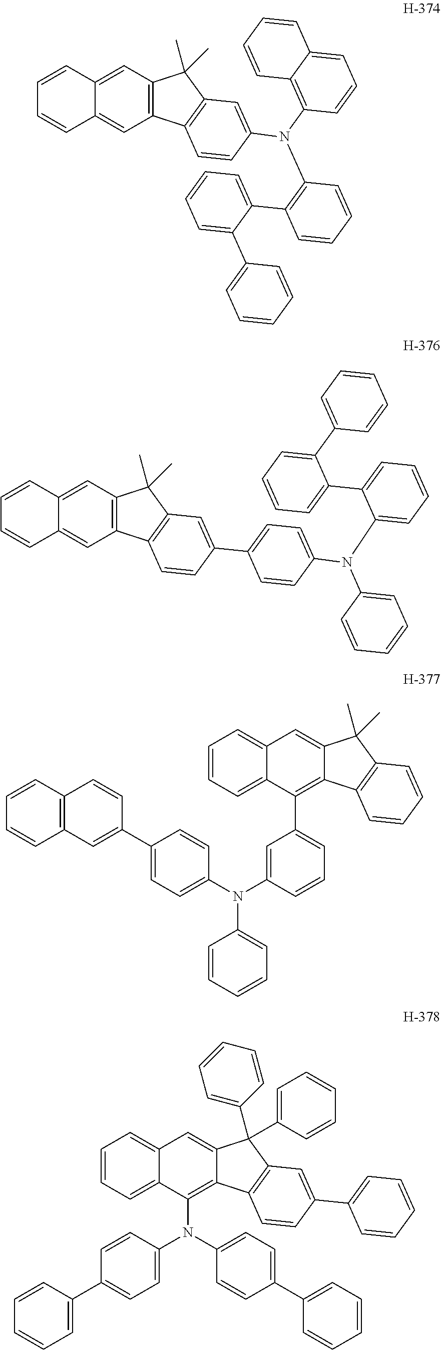 [SMILES: CC1(C)c2cc(N(c3ccccc3-c3ccccc3-c3ccccc3)c3cccc4ccccc34)ccc2-c2cc3ccccc3cc21 ; CC1(C)c2cc(-c3ccc(N(c4ccccc4)c4ccccc4-c4ccccc4-c4ccccc4)cc3)ccc2-c2cc3ccccc3cc21 ; CC1(C)c2ccccc2-c2c1cc1ccccc1c2-c1cccc(N(c2ccccc2)c2ccc(-c3ccc4ccccc4c3)cc2)c1 ; c1ccc(-c2ccc(N(c3ccc(-c4ccccc4)cc3)c3c4c(cc5ccccc35)C(c3ccccc3)(c3ccccc3)c3cc(-c5ccccc5)ccc3-4)cc2)cc1]

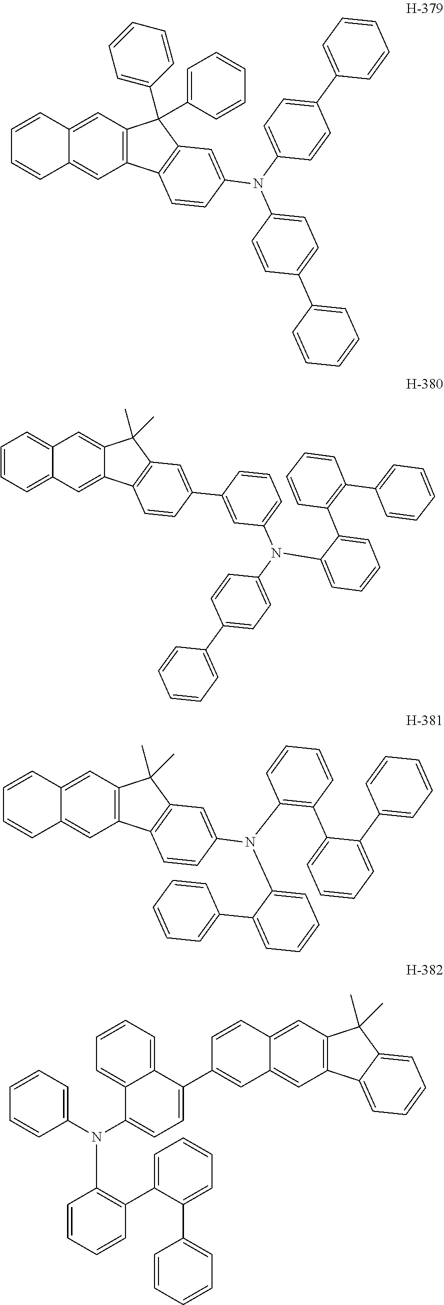 [SMILES: c1ccc(-c2ccc(N(c3ccc(-c4ccccc4)cc3)c3ccc4c(c3)C(c3ccccc3)(c3ccccc3)c3cc5ccccc5cc3-4)cc2)cc1 ; CC1(C)c2cc(-c3cccc(N(c4ccc(-c5ccccc5)cc4)c4ccccc4-c4ccccc4-c4ccccc4)c3)ccc2-c2cc3ccccc3cc21 ; CC1(C)c2cc(N(c3ccccc3-c3ccccc3)c3ccccc3-c3ccccc3-c3ccccc3)ccc2-c2cc3ccccc3cc21 ; CC1(C)c2ccccc2-c2cc3cc(-c4ccc(N(c5ccccc5)c5ccccc5-c5ccccc5-c5ccccc5)c5ccccc45)ccc3cc21]

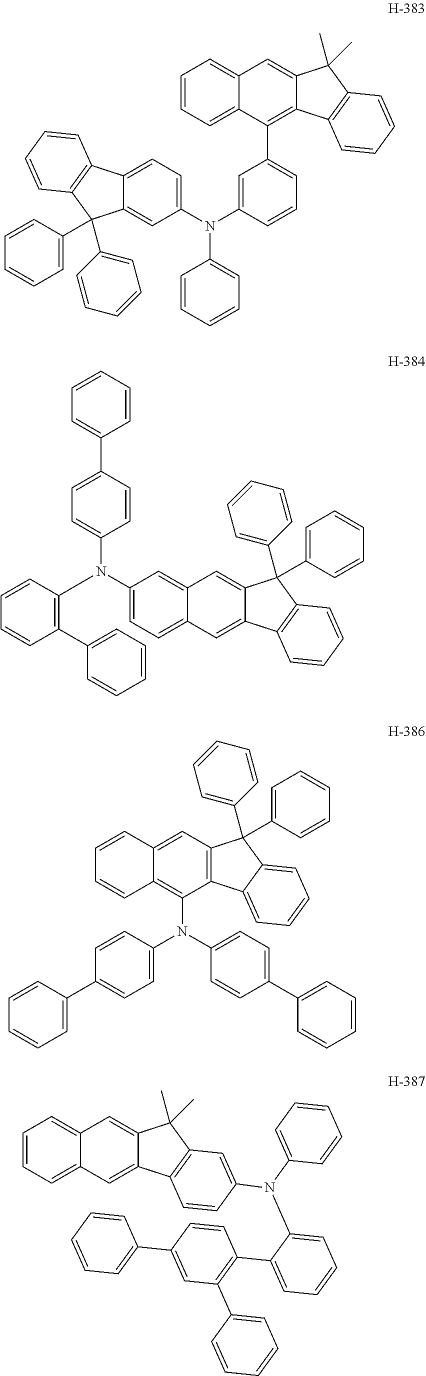 [SMILES: CC1(C)c2ccccc2-c2c1cc1ccccc1c2-c1cccc(N(c2ccccc2)c2ccc3c(c2)C(c2ccccc2)(c2ccccc2)c2ccccc2-3)c1 ; c1ccc(-c2ccc(N(c3ccc4cc5c(cc4c3)C(c3ccccc3)(c3ccccc3)c3ccccc3-5)c3ccccc3-c3ccccc3)cc2)cc1 ; c1ccc(-c2ccc(N(c3ccc(-c4ccccc4)cc3)c3c4c(cc5ccccc35)C(c3ccccc3)(c3ccccc3)c3ccccc3-4)cc2)cc1 ; CC1(C)c2cc(N(c3ccccc3)c3ccccc3-c3ccc(-c4ccccc4)cc3-c3ccccc3)ccc2-c2cc3ccccc3cc21]

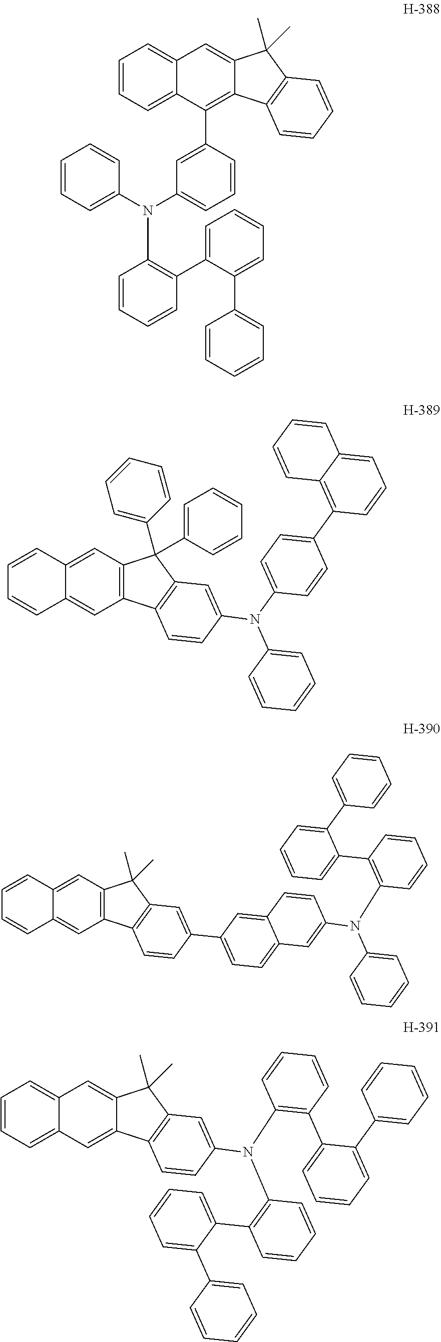 [SMILES: CC1(C)c2ccccc2-c2c1cc1ccccc1c2-c1cccc(N(c2ccccc2)c2ccccc2-c2ccccc2-c2ccccc2)c1 ; c1ccc(N(c2ccc(-c3cccc4ccccc34)cc2)c2ccc3c(c2)C(c2ccccc2)(c2ccccc2)c2cc4ccccc4cc2-3)cc1 ; CC1(C)c2cc(-c3ccc4cc(N(c5ccccc5)c5ccccc5-c5ccccc5-c5ccccc5)ccc4c3)ccc2-c2cc3ccccc3cc21 ; CC1(C)c2cc(N(c3ccccc3-c3ccccc3-c3ccccc3)c3ccccc3-c3ccccc3-c3ccccc3)ccc2-c2cc3ccccc3cc21]

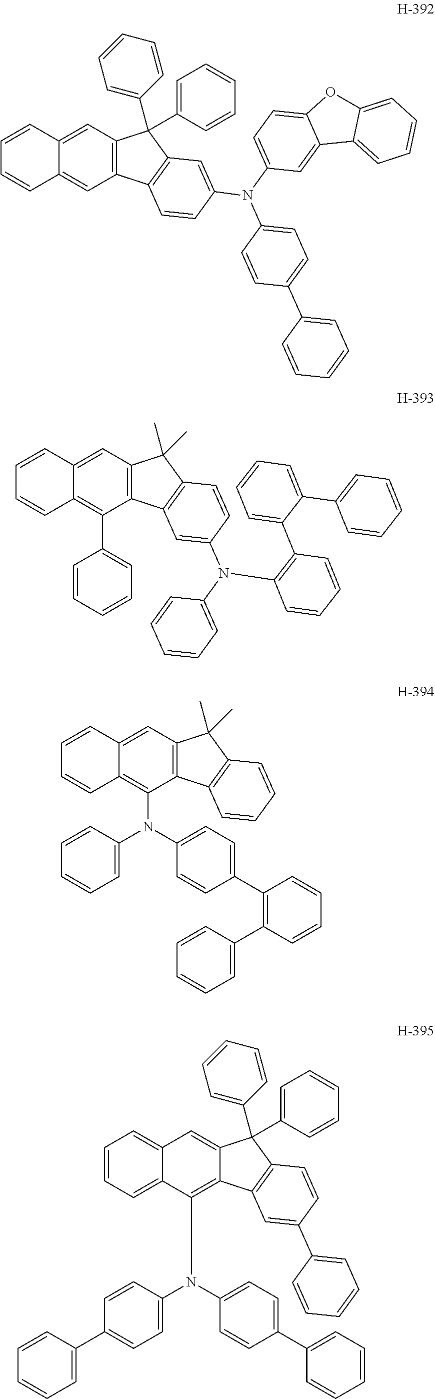

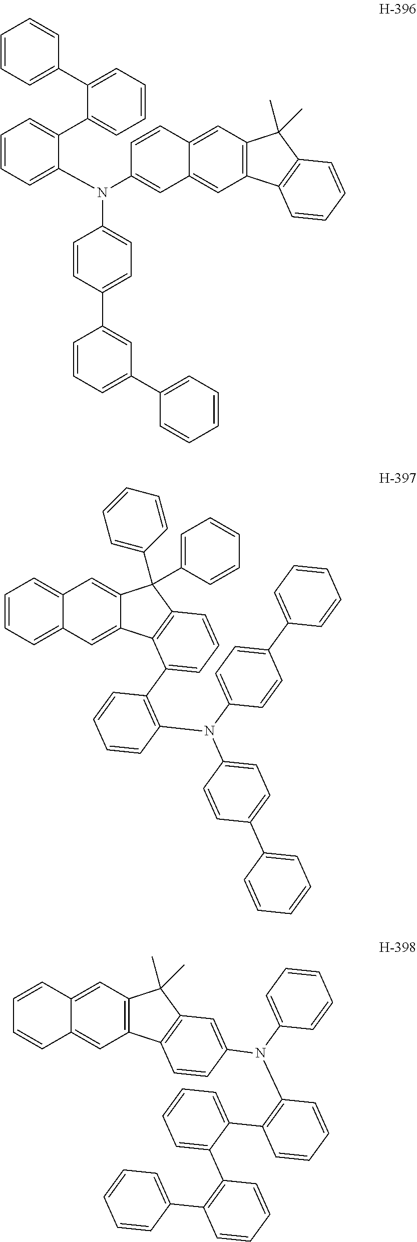 [SMILES: CC1(C)c2ccccc2-c2cc3cc(N(c4ccc(-c5cccc(-c6ccccc6)c5)cc4)c4ccccc4-c4ccccc4-c4ccccc4)ccc3cc21 ; c1ccc(-c2ccc(N(c3ccc(-c4ccccc4)cc3)c3ccccc3-c3cccc4c3-c3cc5ccccc5cc3C4(c3ccccc3)c3ccccc3)cc2)cc1 ; CC1(C)c2cc(N(c3ccccc3)c3ccccc3-c3ccccc3-c3ccccc3-c3ccccc3)ccc2-c2cc3ccccc3cc21]

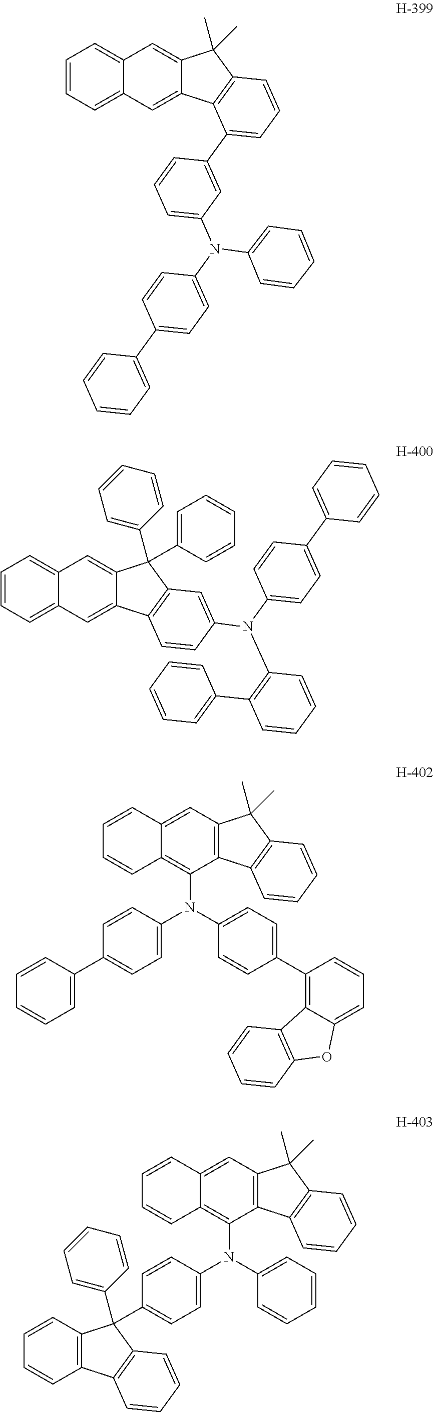

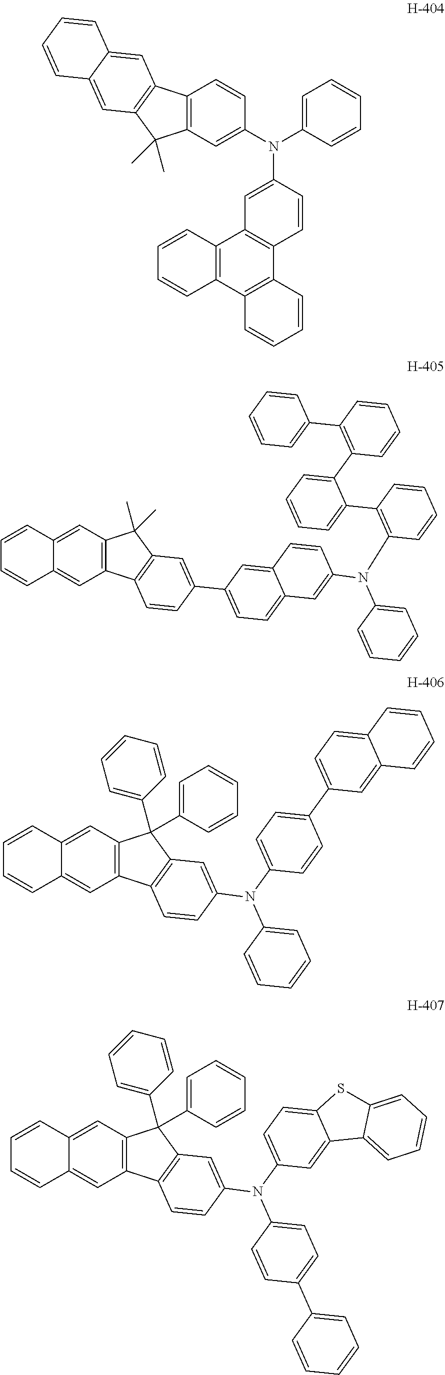

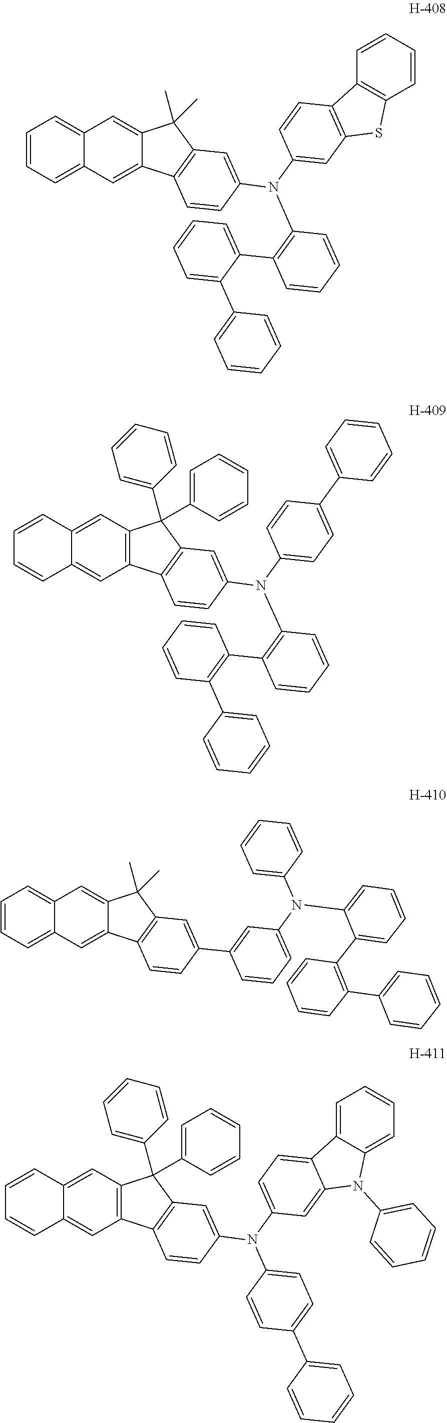 [SMILES: CC1(C)c2cc(N(c3ccc4c(c3)sc3ccccc34)c3ccccc3-c3ccccc3-c3ccccc3)ccc2-c2cc3ccccc3cc21 ; c1ccc(-c2ccc(N(c3ccc4c(c3)C(c3ccccc3)(c3ccccc3)c3cc5ccccc5cc3-4)c3ccccc3-c3ccccc3-c3ccccc3)cc2)cc1 ; CC1(C)c2cc(-c3cccc(N(c4ccccc4)c4ccccc4-c4ccccc4-c4ccccc4)c3)ccc2-c2cc3ccccc3cc21 ; c1ccc(-c2ccc(N(c3ccc4c(c3)C(c3ccccc3)(c3ccccc3)c3cc5ccccc5cc3-4)c3ccc4c5ccccc5n(-c5ccccc5)c4c3)cc2)cc1]

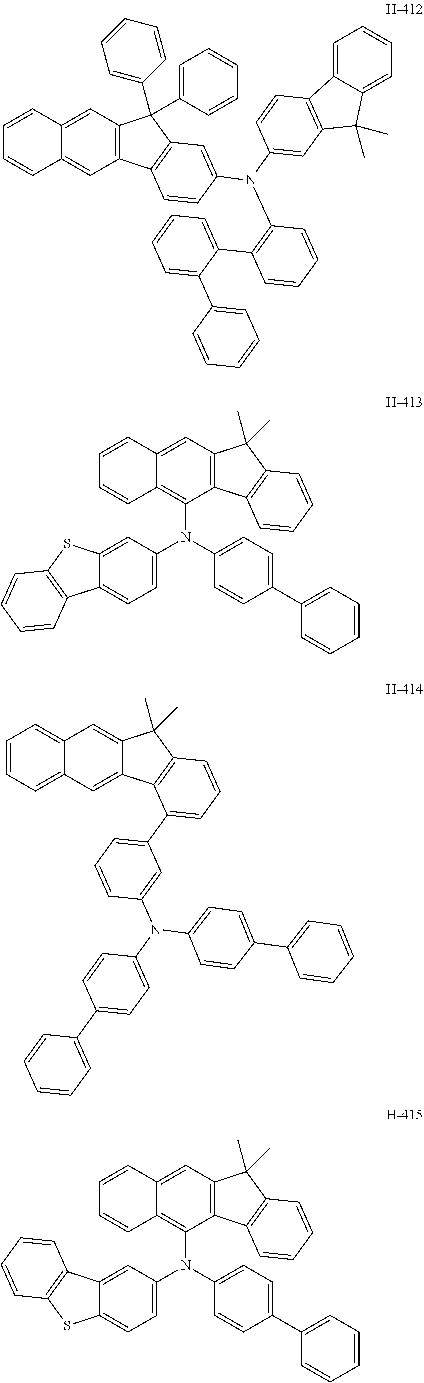

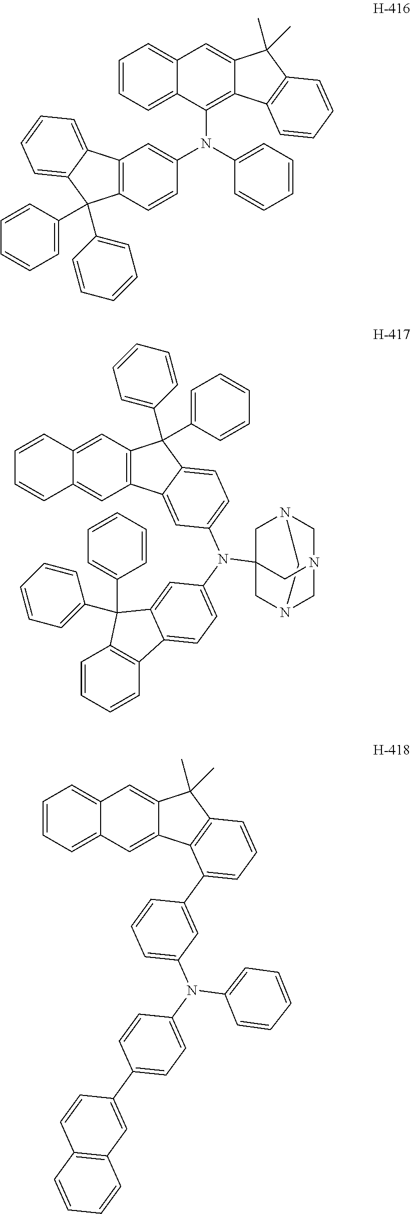 [SMILES: CC1(C)c2ccccc2-c2c1cc1ccccc1c2N(c1ccccc1)c1ccc2c(c1)-c1ccccc1C2(c1ccccc1)c1ccccc1 ; c1ccc(C2(c3ccccc3)c3ccccc3-c3ccc(N(c4ccc5c(c4)-c4cc6ccccc6cc4C5(c4ccccc4)c4ccccc4)C45CN6CN(CN(C6)C4)C5)cc32)cc1 ; CC1(C)c2cc3ccccc3cc2-c2c(-c3cccc(N(c4ccccc4)c4ccc(-c5ccc6ccccc6c5)cc4)c3)cccc21]

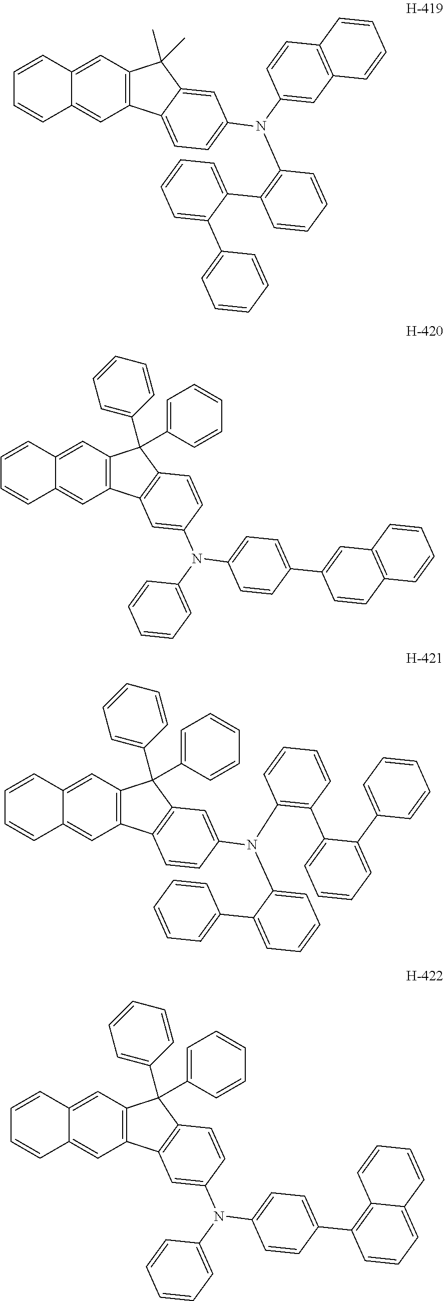 [SMILES: CC1(C)c2cc(N(c3ccc4ccccc4c3)c3ccccc3-c3ccccc3-c3ccccc3)ccc2-c2cc3ccccc3cc21 ; c1ccc(N(c2ccc(-c3ccc4ccccc4c3)cc2)c2ccc3c(c2)-c2cc4ccccc4cc2C3(c2ccccc2)c2ccccc2)cc1 ; c1ccc(-c2ccccc2-c2ccccc2N(c2ccc3c(c2)C(c2ccccc2)(c2ccccc2)c2cc4ccccc4cc2-3)c2ccccc2-c2ccccc2)cc1 ; c1ccc(N(c2ccc(-c3cccc4ccccc34)cc2)c2ccc3c(c2)-c2cc4ccccc4cc2C3(c2ccccc2)c2ccccc2)cc1]

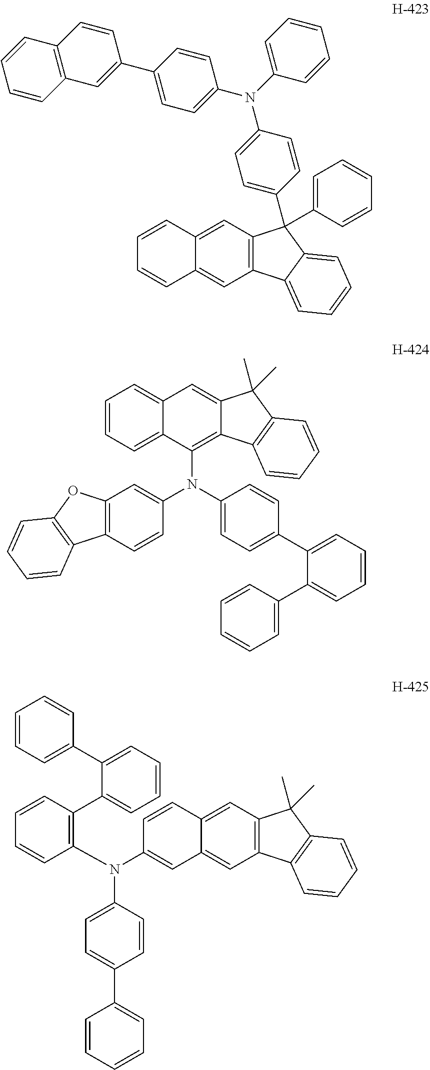 [SMILES: c1ccc(N(c2ccc(-c3ccc4ccccc4c3)cc2)c2ccc(C3(c4ccccc4)c4ccccc4-c4cc5ccccc5cc43)cc2)cc1 ; CC1(C)c2ccccc2-c2c1cc1ccccc1c2N(c1ccc(-c2ccccc2-c2ccccc2)cc1)c1ccc2c(c1)oc1ccccc12 ; CC1(C)c2ccccc2-c2cc3cc(N(c4ccc(-c5ccccc5)cc4)c4ccccc4-c4ccccc4-c4ccccc4)ccc3cc21]

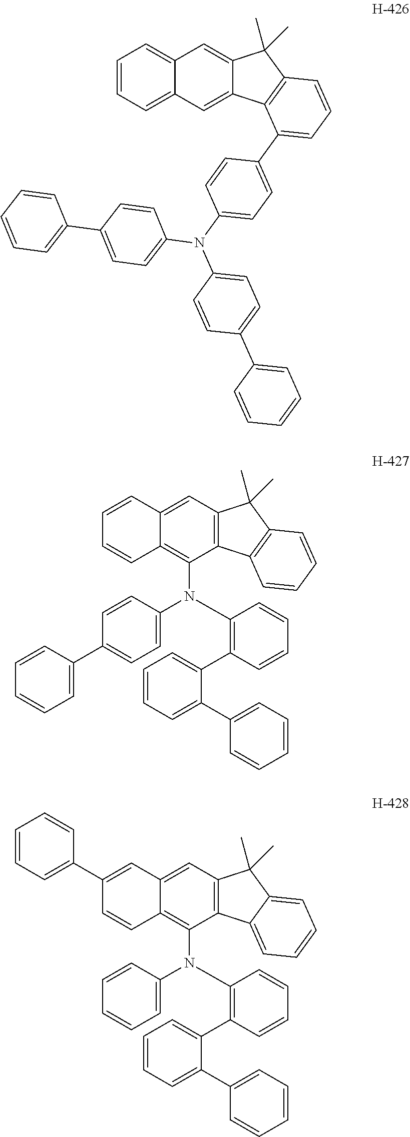 [SMILES: CC1(C)c2cc3ccccc3cc2-c2c(-c3ccc(N(c4ccc(-c5ccccc5)cc4)c4ccc(-c5ccccc5)cc4)cc3)cccc21 ; CC1(C)c2ccccc2-c2c1cc1ccccc1c2N(c1ccc(-c2ccccc2)cc1)c1ccccc1-c1ccccc1-c1ccccc1 ; CC1(C)c2ccccc2-c2c1cc1cc(-c3ccccc3)ccc1c2N(c1ccccc1)c1ccccc1-c1ccccc1-c1ccccc1]

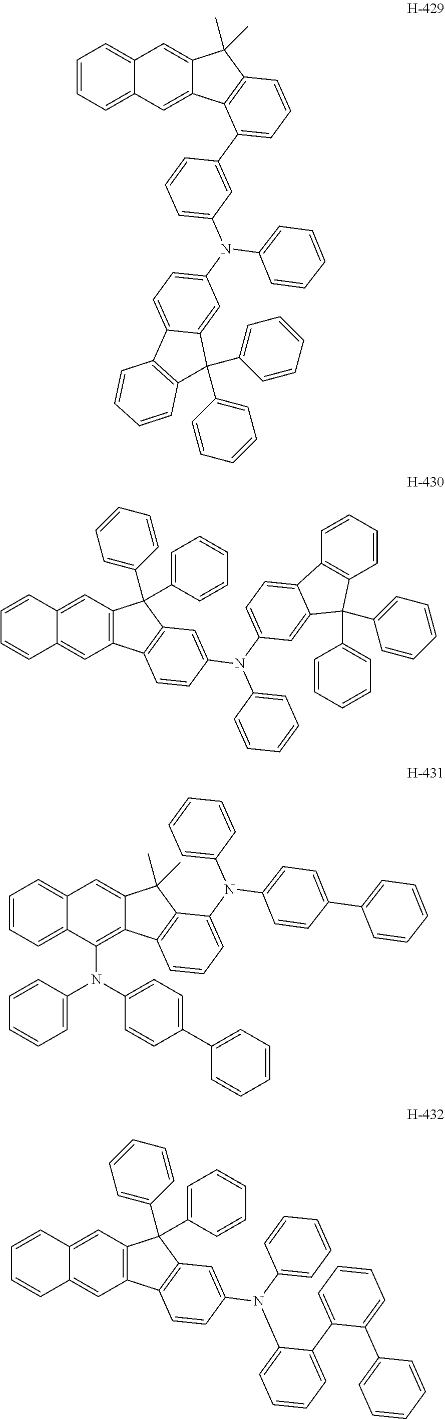 [SMILES: CC1(C)c2cc3ccccc3cc2-c2c(-c3cccc(N(c4ccccc4)c4ccc5c(c4)C(c4ccccc4)(c4ccccc4)c4ccccc4-5)c3)cccc21 ; c1ccc(N(c2ccc3c(c2)C(c2ccccc2)(c2ccccc2)c2ccccc2-3)c2ccc3c(c2)C(c2ccccc2)(c2ccccc2)c2cc4ccccc4cc2-3)cc1 ; CC1(C)c2cc3ccccc3c(N(c3ccccc3)c3ccc(-c4ccccc4)cc3)c2-c2cccc(N(c3ccccc3)c3ccc(-c4ccccc4)cc3)c21 ; c1ccc(-c2ccccc2-c2ccccc2N(c2ccccc2)c2ccc3c(c2)C(c2ccccc2)(c2ccccc2)c2cc4ccccc4cc2-3)cc1]

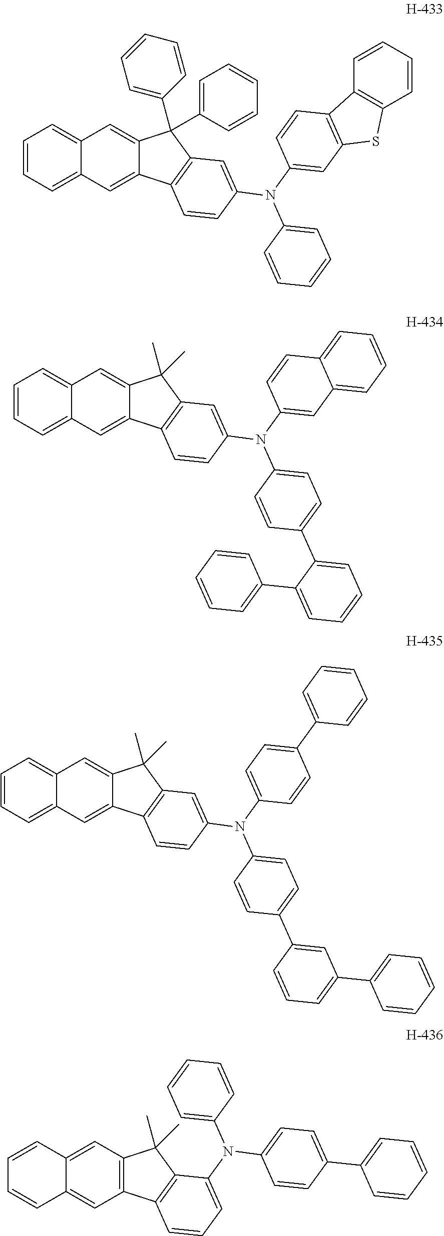 [SMILES: c1ccc(N(c2ccc3c(c2)C(c2ccccc2)(c2ccccc2)c2cc4ccccc4cc2-3)c2ccc3c(c2)sc2ccccc23)cc1 ; CC1(C)c2cc(N(c3ccc(-c4ccccc4-c4ccccc4)cc3)c3ccc4ccccc4c3)ccc2-c2cc3ccccc3cc21 ; CC1(C)c2cc(N(c3ccc(-c4ccccc4)cc3)c3ccc(-c4cccc(-c5ccccc5)c4)cc3)ccc2-c2cc3ccccc3cc21 ; CC1(C)c2cc3ccccc3cc2-c2cccc(N(c3ccccc3)c3ccc(-c4ccccc4)cc3)c21]

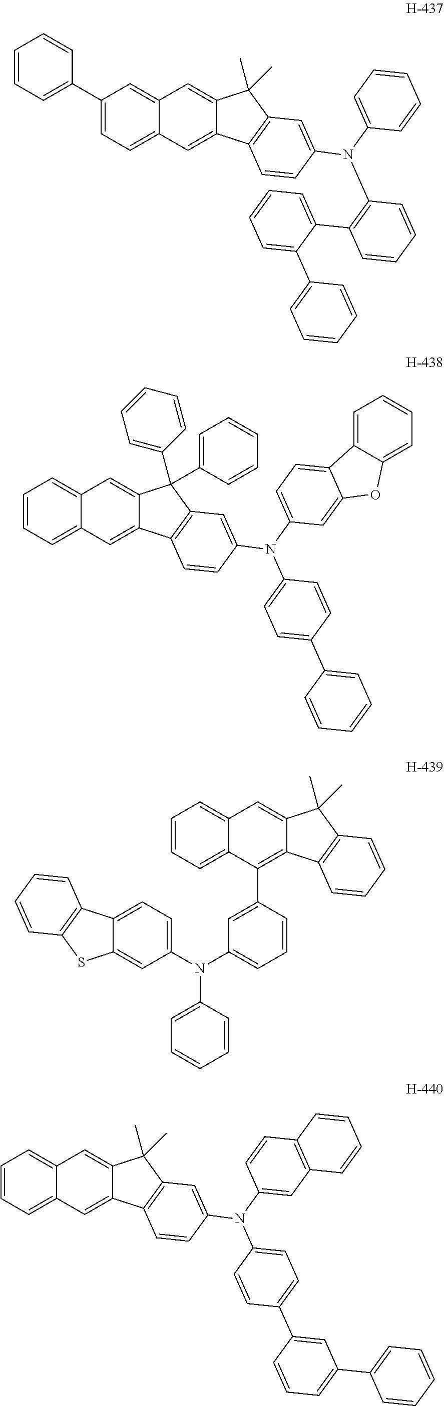 [SMILES: CC1(C)c2cc(N(c3ccccc3)c3ccccc3-c3ccccc3-c3ccccc3)ccc2-c2cc3ccc(-c4ccccc4)cc3cc21 ; c1ccc(-c2ccc(N(c3ccc4c(c3)C(c3ccccc3)(c3ccccc3)c3cc5ccccc5cc3-4)c3ccc4c(c3)oc3ccccc34)cc2)cc1 ; CC1(C)c2ccccc2-c2c1cc1ccccc1c2-c1cccc(N(c2ccccc2)c2ccc3c(c2)sc2ccccc23)c1 ; CC1(C)c2cc(N(c3ccc(-c4cccc(-c5ccccc5)c4)cc3)c3ccc4ccccc4c3)ccc2-c2cc3ccccc3cc21]

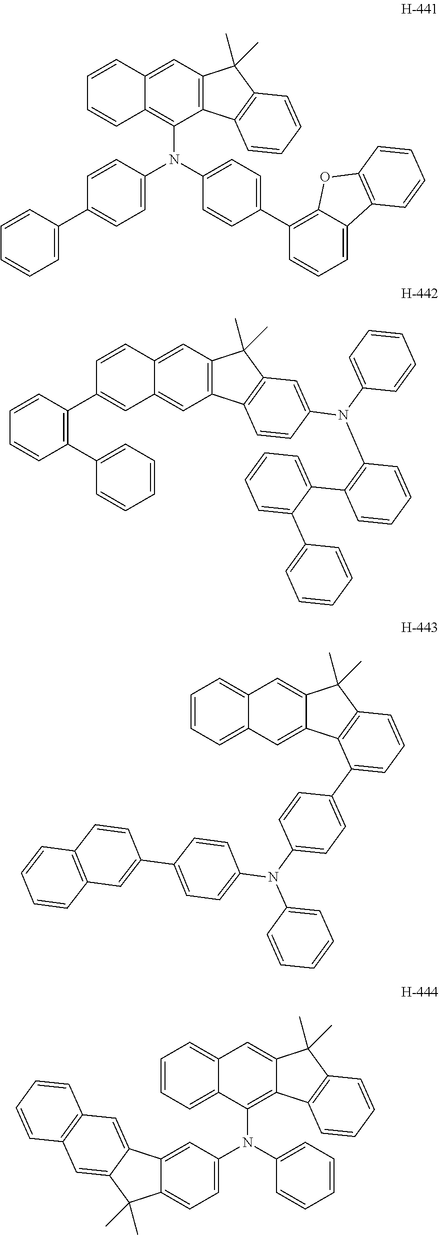 [SMILES: CC1(C)c2ccccc2-c2c1cc1ccccc1c2N(c1ccc(-c2ccccc2)cc1)c1ccc(-c2cccc3c2oc2ccccc23)cc1 ; CC1(C)c2cc(N(c3ccccc3)c3ccccc3-c3ccccc3-c3ccccc3)ccc2-c2cc3cc(-c4ccccc4-c4ccccc4)ccc3cc21 ; CC1(C)c2cc3ccccc3cc2-c2c(-c3ccc(N(c4ccccc4)c4ccc(-c5ccc6ccccc6c5)cc4)cc3)cccc21 ; CC1(C)c2ccc(N(c3ccccc3)c3c4c(cc5ccccc35)C(C)(C)c3ccccc3-4)cc2-c2cc3ccccc3cc21]

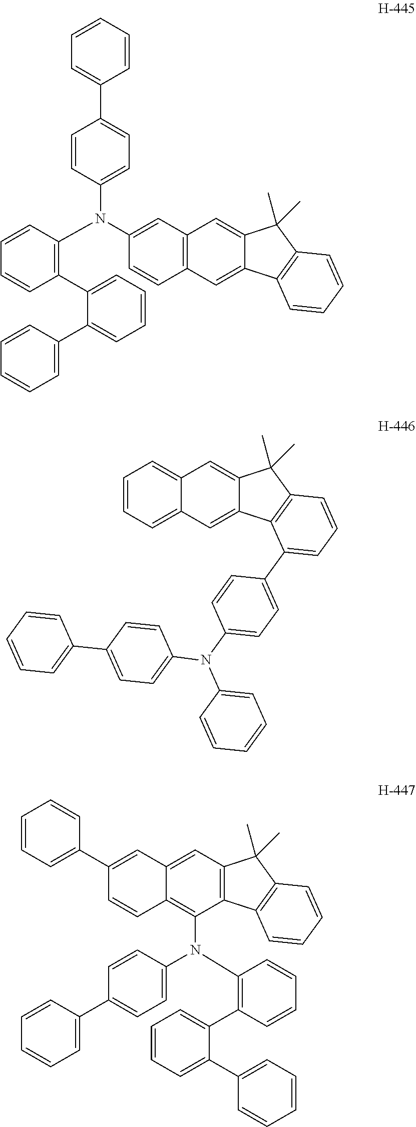 [SMILES: CC1(C)c2ccccc2-c2cc3ccc(N(c4ccc(-c5ccccc5)cc4)c4ccccc4-c4ccccc4-c4ccccc4)cc3cc21 ; CC1(C)c2cc3ccccc3cc2-c2c(-c3ccc(N(c4ccccc4)c4ccc(-c5ccccc5)cc4)cc3)cccc21 ; CC1(C)c2ccccc2-c2c1cc1cc(-c3ccccc3)ccc1c2N(c1ccc(-c2ccccc2)cc1)c1ccccc1-c1ccccc1-c1ccccc1]

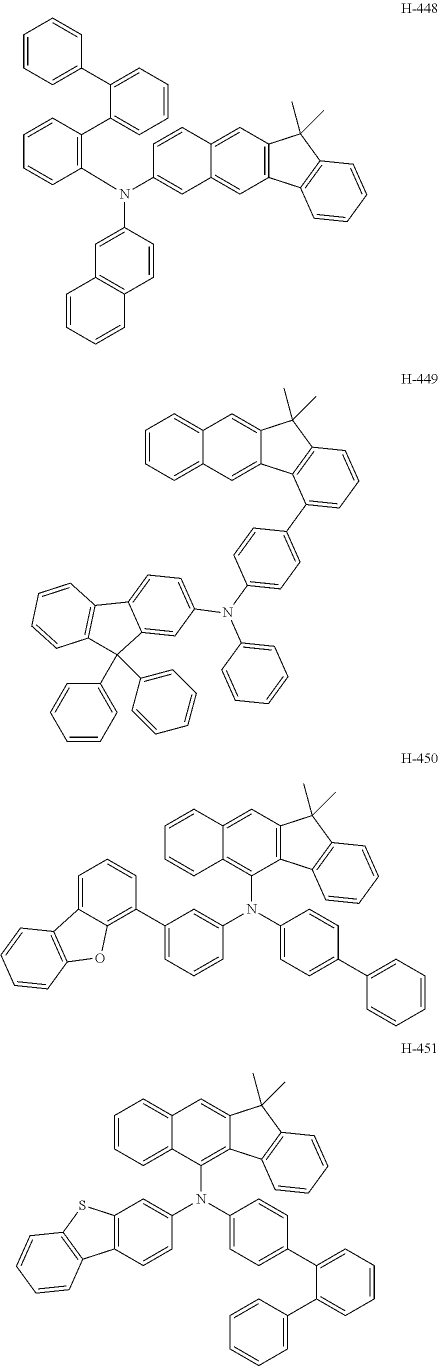 [SMILES: CC1(C)c2ccccc2-c2cc3cc(N(c4ccc5ccccc5c4)c4ccccc4-c4ccccc4-c4ccccc4)ccc3cc21 ; CC1(C)c2cc3ccccc3cc2-c2c(-c3ccc(N(c4ccccc4)c4ccc5c(c4)C(c4ccccc4)(c4ccccc4)c4ccccc4-5)cc3)cccc21 ; CC1(C)c2ccccc2-c2c1cc1ccccc1c2N(c1ccc(-c2ccccc2)cc1)c1cccc(-c2cccc3c2oc2ccccc23)c1 ; CC1(C)c2ccccc2-c2c1cc1ccccc1c2N(c1ccc(-c2ccccc2-c2ccccc2)cc1)c1ccc2c(c1)sc1ccccc12]

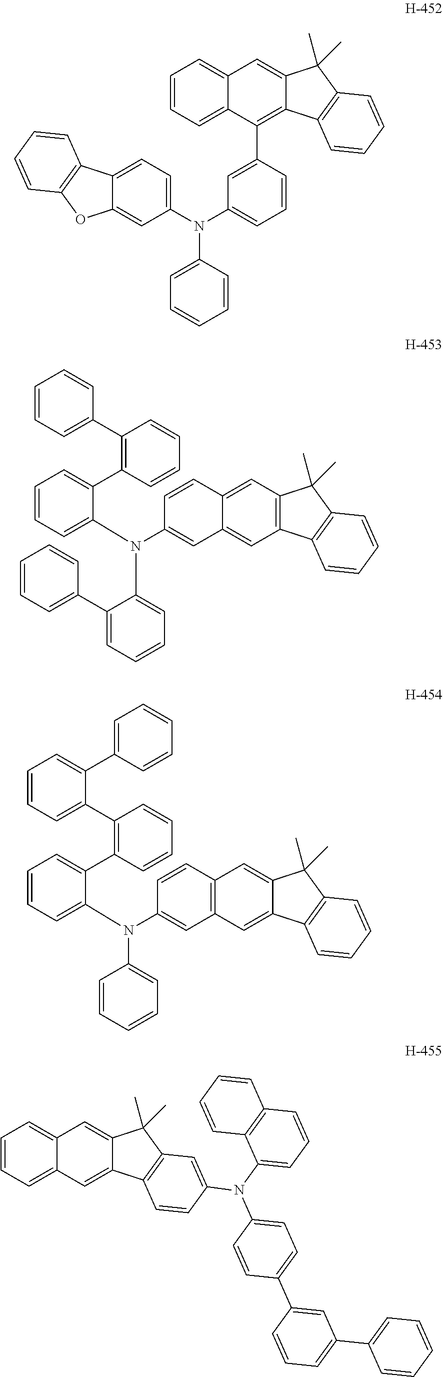

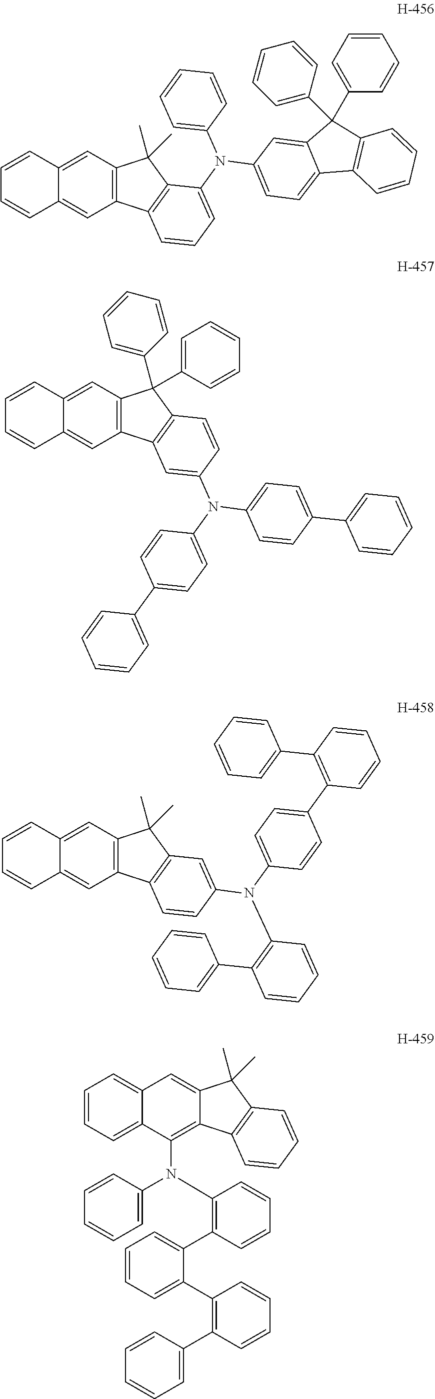 [SMILES: CC1(C)c2cc3ccccc3cc2-c2cccc(N(c3ccccc3)c3ccc4c(c3)C(c3ccccc3)(c3ccccc3)c3ccccc3-4)c21 ; c1ccc(-c2ccc(N(c3ccc(-c4ccccc4)cc3)c3ccc4c(c3)-c3cc5ccccc5cc3C4(c3ccccc3)c3ccccc3)cc2)cc1 ; CC1(C)c2cc(N(c3ccc(-c4ccccc4-c4ccccc4)cc3)c3ccccc3-c3ccccc3)ccc2-c2cc3ccccc3cc21 ; CC1(C)c2ccccc2-c2c1cc1ccccc1c2N(c1ccccc1)c1ccccc1-c1ccccc1-c1ccccc1-c1ccccc1]

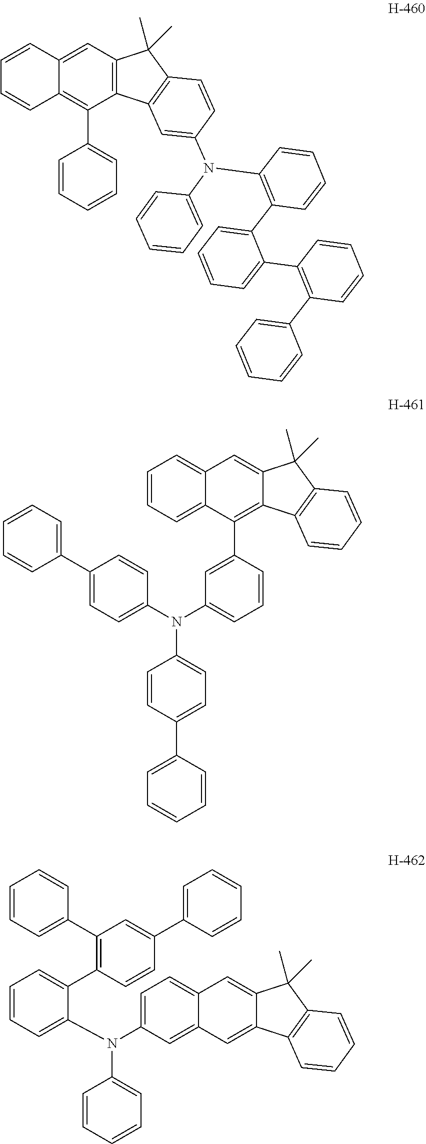 [SMILES: CC1(C)c2ccc(N(c3ccccc3)c3ccccc3-c3ccccc3-c3ccccc3-c3ccccc3)cc2-c2c1cc1ccccc1c2-c1ccccc1 ; CC1(C)c2ccccc2-c2c1cc1ccccc1c2-c1cccc(N(c2ccc(-c3ccccc3)cc2)c2ccc(-c3ccccc3)cc2)c1 ; CC1(C)c2ccccc2-c2cc3cc(N(c4ccccc4)c4ccccc4-c4ccc(-c5ccccc5)cc4-c4ccccc4)ccc3cc21]

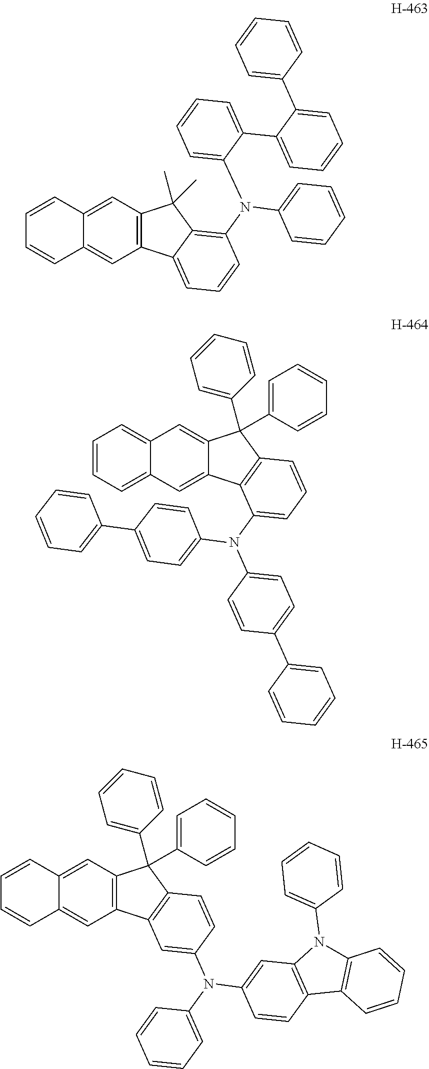

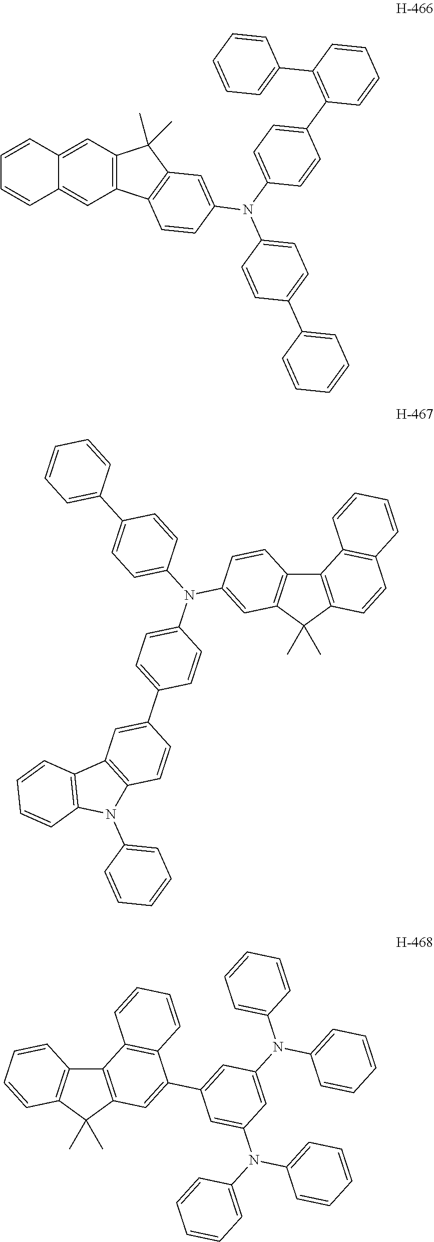 [SMILES: CC1(C)c2cc(N(c3ccc(-c4ccccc4)cc3)c3ccc(-c4ccccc4-c4ccccc4)cc3)ccc2-c2cc3ccccc3cc21 ; CC1(C)c2cc(N(c3ccc(-c4ccccc4)cc3)c3ccc(-c4ccc5c(c4)c4ccccc4n5-c4ccccc4)cc3)ccc2-c2c1ccc1ccccc21 ; CC1(C)c2ccccc2-c2c1cc(-c1cc(N(c3ccccc3)c3ccccc3)cc(N(c3ccccc3)c3ccccc3)c1)c1ccccc21]

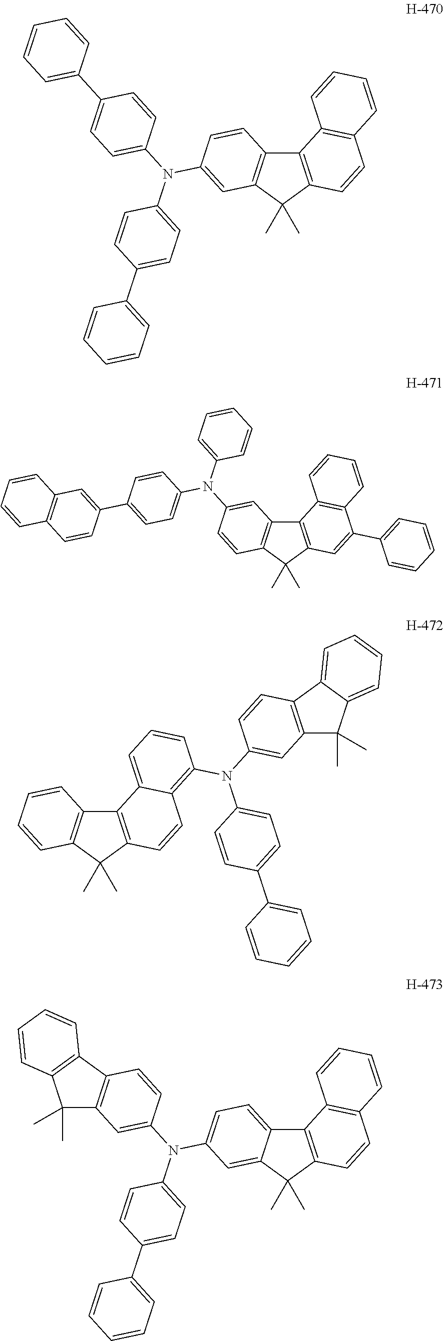 [SMILES: CC1(C)c2cc(N(c3ccc(-c4ccccc4)cc3)c3ccc(-c4ccccc4)cc3)ccc2-c2c1ccc1ccccc21 ; CC1(C)c2ccc(N(c3ccccc3)c3ccc(-c4ccc5ccccc5c4)cc3)cc2-c2c1cc(-c1ccccc1)c1ccccc21 ; CC1(C)c2ccccc2-c2ccc(N(c3ccc(-c4ccccc4)cc3)c3cccc4c5c(ccc34)C(C)(C)c3ccccc3-5)cc21 ; CC1(C)c2ccccc2-c2ccc(N(c3ccc(-c4ccccc4)cc3)c3ccc4c(c3)C(C)(C)c3ccc5ccccc5c3-4)cc21]

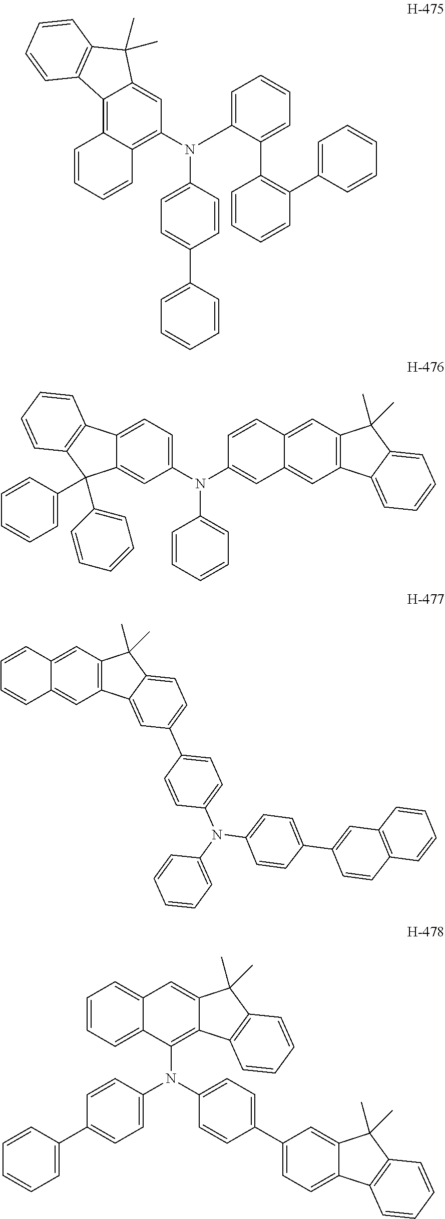 [SMILES: CC1(C)c2ccccc2-c2c1cc(N(c1ccc(-c3ccccc3)cc1)c1ccccc1-c1ccccc1-c1ccccc1)c1ccccc21 ; CC1(C)c2ccccc2-c2cc3cc(N(c4ccccc4)c4ccc5c(c4)C(c4ccccc4)(c4ccccc4)c4ccccc4-5)ccc3cc21 ; CC1(C)c2ccc(-c3ccc(N(c4ccccc4)c4ccc(-c5ccc6ccccc6c5)cc4)cc3)cc2-c2cc3ccccc3cc21 ; CC1(C)c2ccccc2-c2ccc(-c3ccc(N(c4ccc(-c5ccccc5)cc4)c4c5c(cc6ccccc46)C(C)(C)c4ccccc4-5)cc3)cc21]

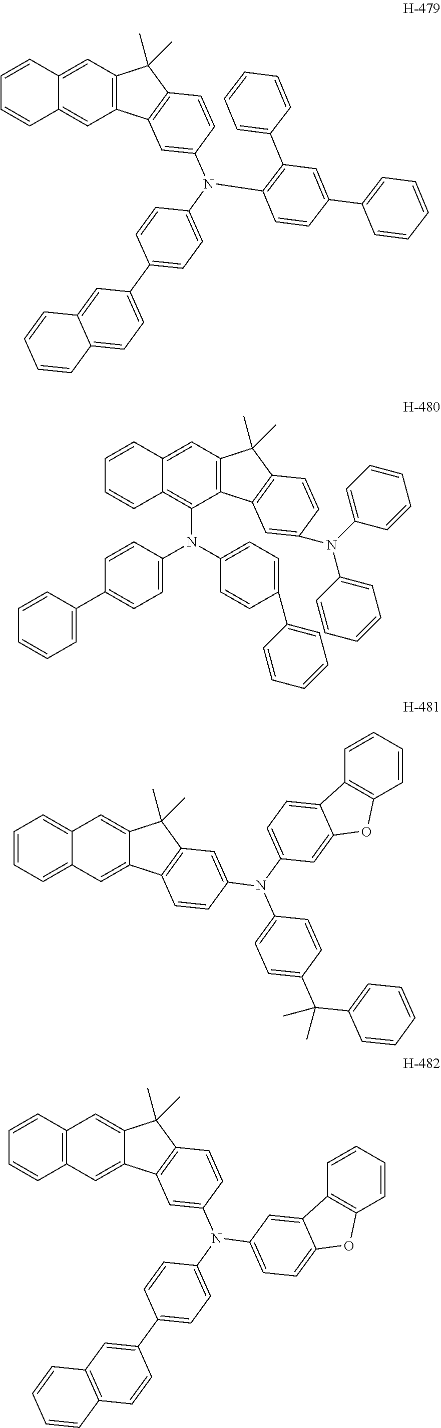 [SMILES: CC1(C)c2ccc(N(c3ccc(-c4ccc5ccccc5c4)cc3)c3ccc(-c4ccccc4)cc3-c3ccccc3)cc2-c2cc3ccccc3cc21 ; CC1(C)c2ccc(N(c3ccccc3)c3ccccc3)cc2-c2c1cc1ccccc1c2N(c1ccc(-c2ccccc2)cc1)c1ccc(-c2ccccc2)cc1 ; CC(C)(c1ccccc1)c1ccc(N(c2ccc3c(c2)C(C)(C)c2cc4ccccc4cc2-3)c2ccc3c(c2)oc2ccccc23)cc1 ; CC1(C)c2ccc(N(c3ccc(-c4ccc5ccccc5c4)cc3)c3ccc4oc5ccccc5c4c3)cc2-c2cc3ccccc3cc21]

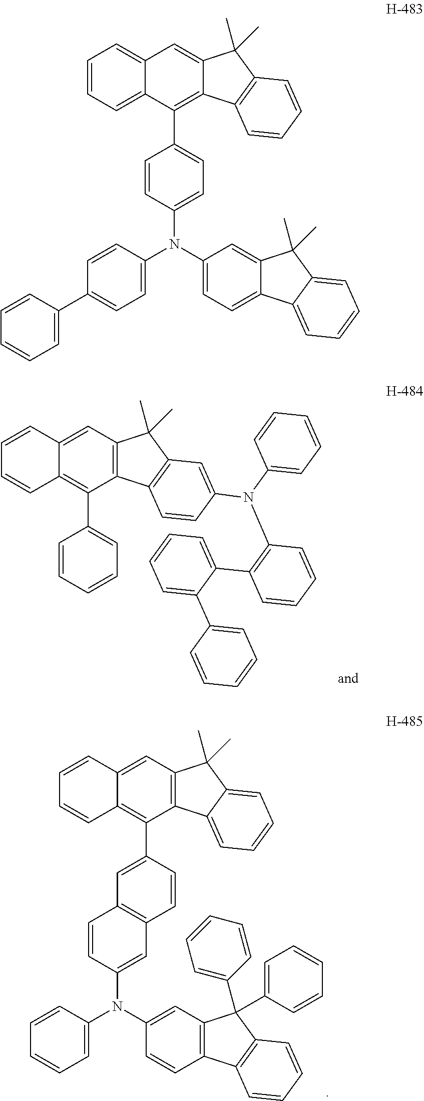 [SMILES: CC1(C)c2ccccc2-c2ccc(N(c3ccc(-c4ccccc4)cc3)c3ccc(-c4c5c(cc6ccccc46)C(C)(C)c4ccccc4-5)cc3)cc21 ; CC1(C)c2cc(N(c3ccccc3)c3ccccc3-c3ccccc3-c3ccccc3)ccc2-c2c1cc1ccccc1c2-c1ccccc1 ; CC1(C)c2ccccc2-c2c1cc1ccccc1c2-c1ccc2cc(N(c3ccccc3)c3ccc4c(c3)C(c3ccccc3)(c3ccccc3)c3ccccc3-4)ccc2c1]

7. The plurality of host materials according to claim 1, wherein the compound represented by the Formula 2 is selected from the following compounds: 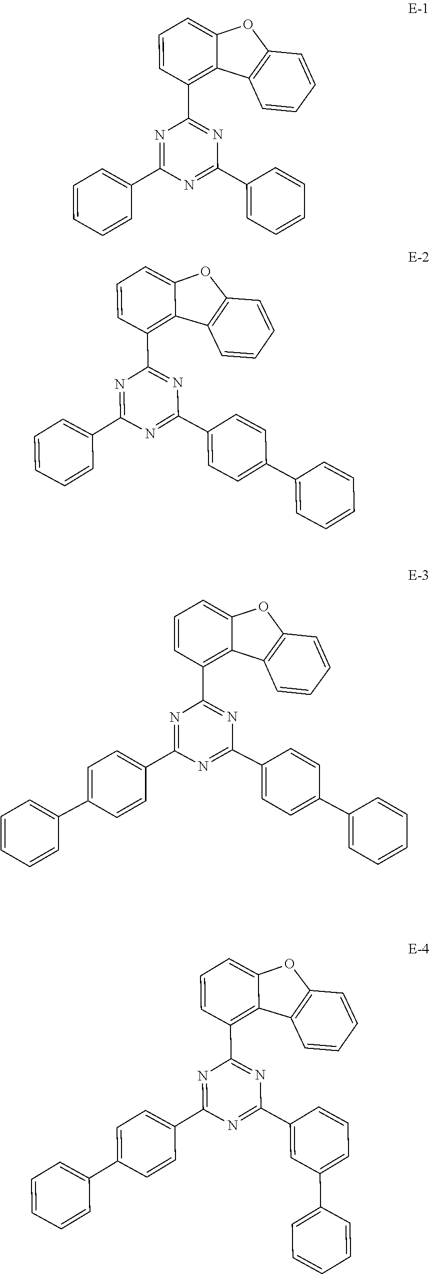 [SMILES: c1ccc(-c2nc(-c3ccccc3)nc(-c3cccc4oc5ccccc5c34)n2)cc1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1] 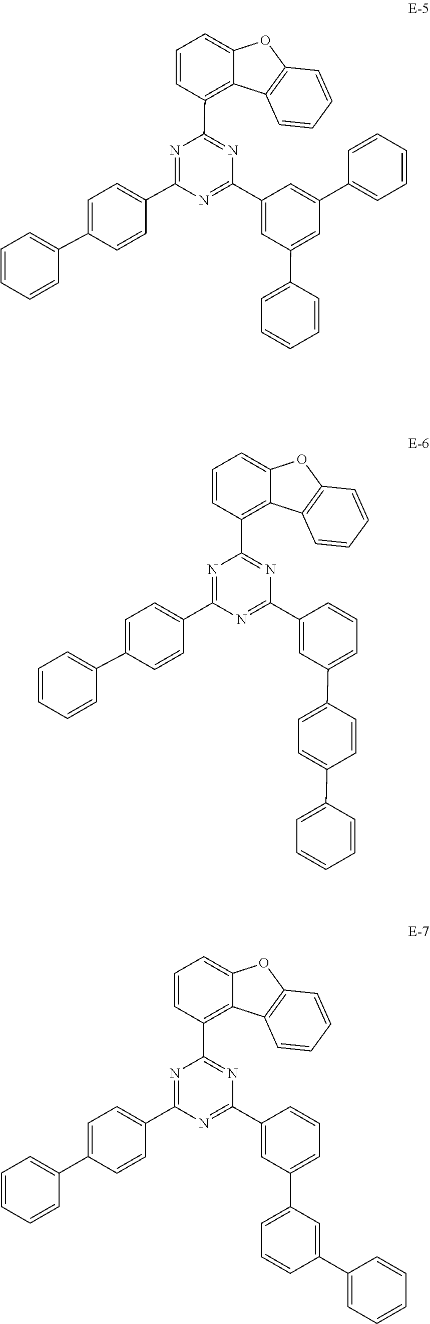 [SMILES: c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cccc(-c4nc(-c5ccc(-c6ccccc6)cc5)nc(-c5cccc6oc7ccccc7c56)n4)c3)cc2)cc1 ; c1ccc(-c2ccc(-c3nc(-c4cccc(-c5cccc(-c6ccccc6)c5)c4)nc(-c4cccc5oc6ccccc6c45)n3)cc2)cc1] 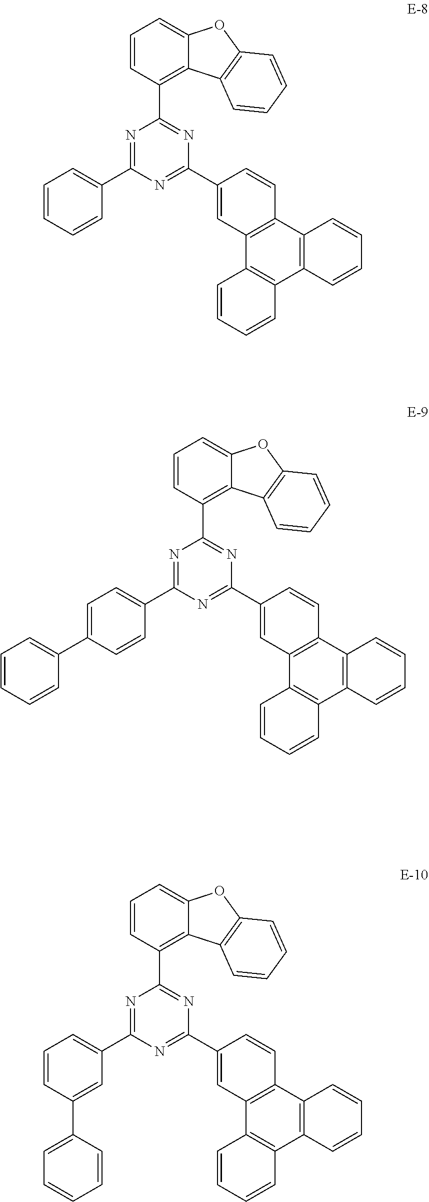 [SMILES: c1ccc(-c2nc(-c3ccc4c5ccccc5c5ccccc5c4c3)nc(-c3cccc4oc5ccccc5c34)n2)cc1 ; c1ccc(-c2ccc(-c3nc(-c4ccc5c6ccccc6c6ccccc6c5c4)nc(-c4cccc5oc6ccccc6c45)n3)cc2)cc1 ; c1ccc(-c2cccc(-c3nc(-c4ccc5c6ccccc6c6ccccc6c5c4)nc(-c4cccc5oc6ccccc6c45)n3)c2)cc1] 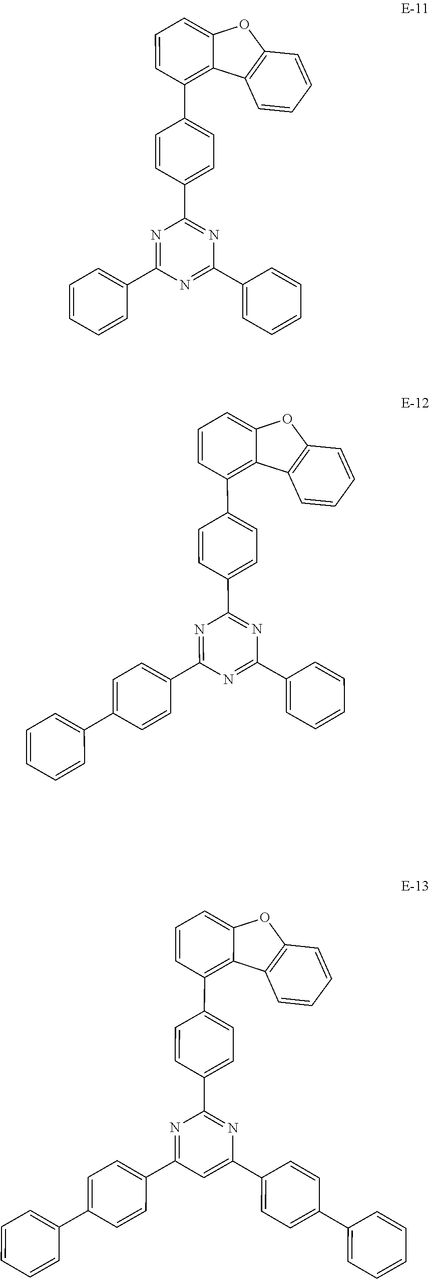 [SMILES: c1ccc(-c2nc(-c3ccccc3)nc(-c3ccc(-c4cccc5oc6ccccc6c45)cc3)n2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3cc(-c4ccc(-c5ccccc5)cc4)nc(-c4ccc(-c5cccc6oc7ccccc7c56)cc4)n3)cc2)cc1] 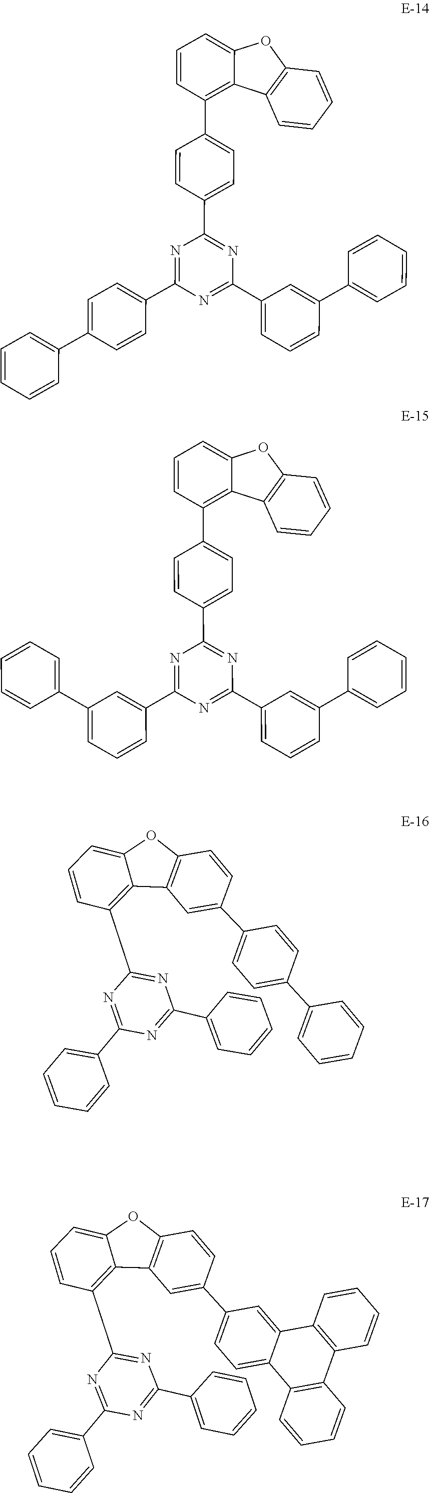 [SMILES: c1ccc(-c2ccc(-c3nc(-c4ccc(-c5cccc6oc7ccccc7c56)cc4)nc(-c4cccc(-c5ccccc5)c4)n3)cc2)cc1 ; c1ccc(-c2cccc(-c3nc(-c4ccc(-c5cccc6oc7ccccc7c56)cc4)nc(-c4cccc(-c5ccccc5)c4)n3)c2)cc1 ; c1ccc(-c2ccc(-c3ccc4oc5cccc(-c6nc(-c7ccccc7)nc(-c7ccccc7)n6)c5c4c3)cc2)cc1 ; c1ccc(-c2nc(-c3ccccc3)nc(-c3cccc4oc5ccc(-c6ccc7c8ccccc8c8ccccc8c7c6)cc5c34)n2)cc1] 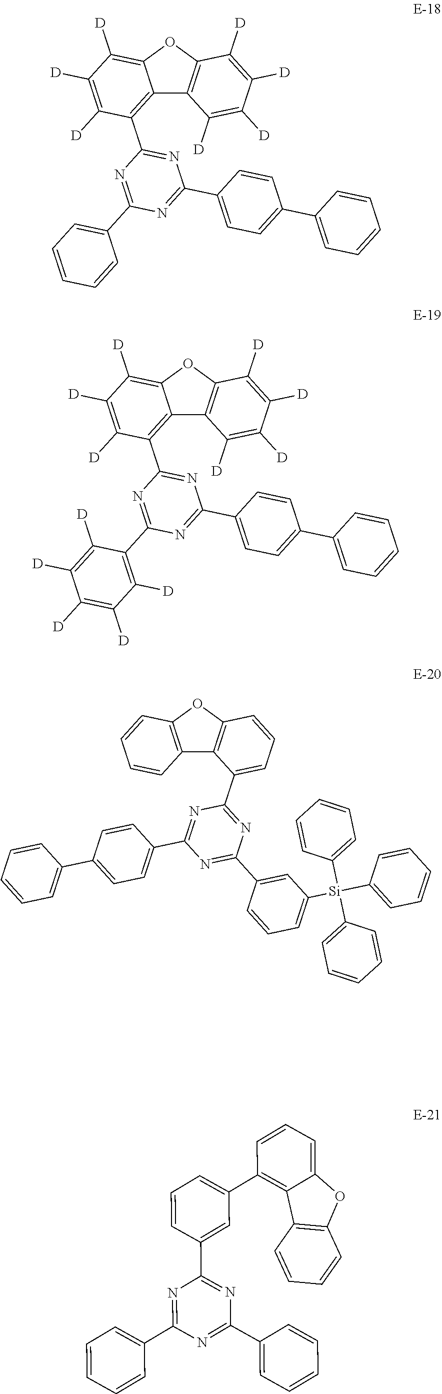 [SMILES: [2H]c1c([2H])c([2H])c2c(oc3c([2H])c([2H])c([2H])c(-c4nc(-c5ccccc5)nc(-c5ccc(-c6ccccc6)cc5)n4)c32)c1[2H] ; [2H]c1c([2H])c([2H])c(-c2nc(-c3ccc(-c4ccccc4)cc3)nc(-c3c([2H])c([2H])c([2H])c4oc5c([2H])c([2H])c([2H])c([2H])c5c34)n2)c([2H])c1[2H] ; c1ccc(-c2ccc(-c3nc(-c4cccc([Si](c5ccccc5)(c5ccccc5)c5ccccc5)c4)nc(-c4cccc5oc6ccccc6c45)n3)cc2)cc1 ; c1ccc(-c2nc(-c3ccccc3)nc(-c3cccc(-c4cccc5oc6ccccc6c45)c3)n2)cc1] 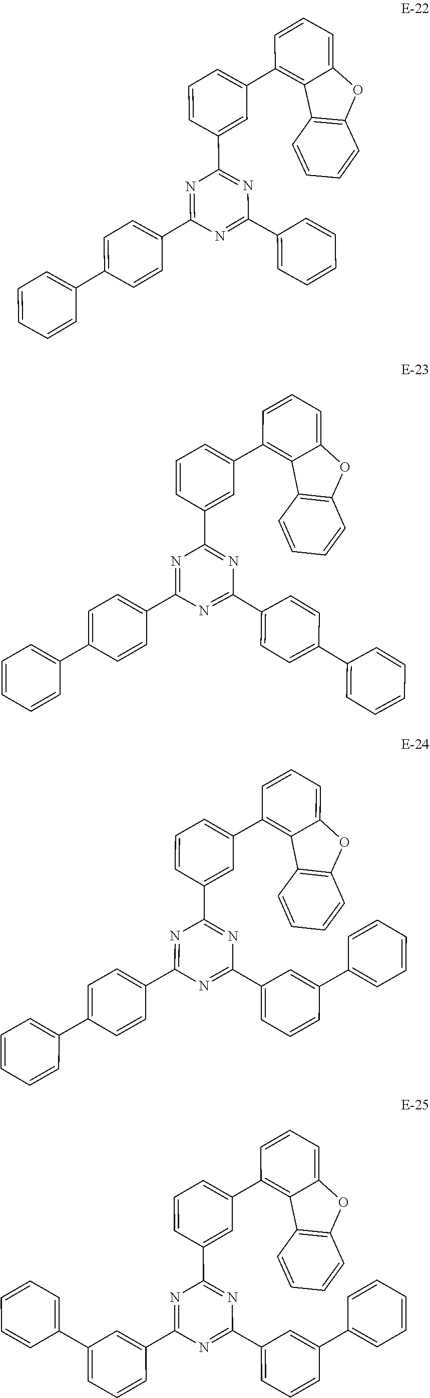 [SMILES: c1ccc(-c2ccc(-c3nc(-c4ccccc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4cccc(-c5ccccc5)c4)nc(-c4cccc(-c5cccc6oc7ccccc7c56)c4)n3)cc2)cc1 ; c1ccc(-c2cccc(-c3nc(-c4cccc(-c5ccccc5)c4)nc(-c4cccc(-c5cccc6oc7ccccc7c56)c4)n3)c2)cc1] 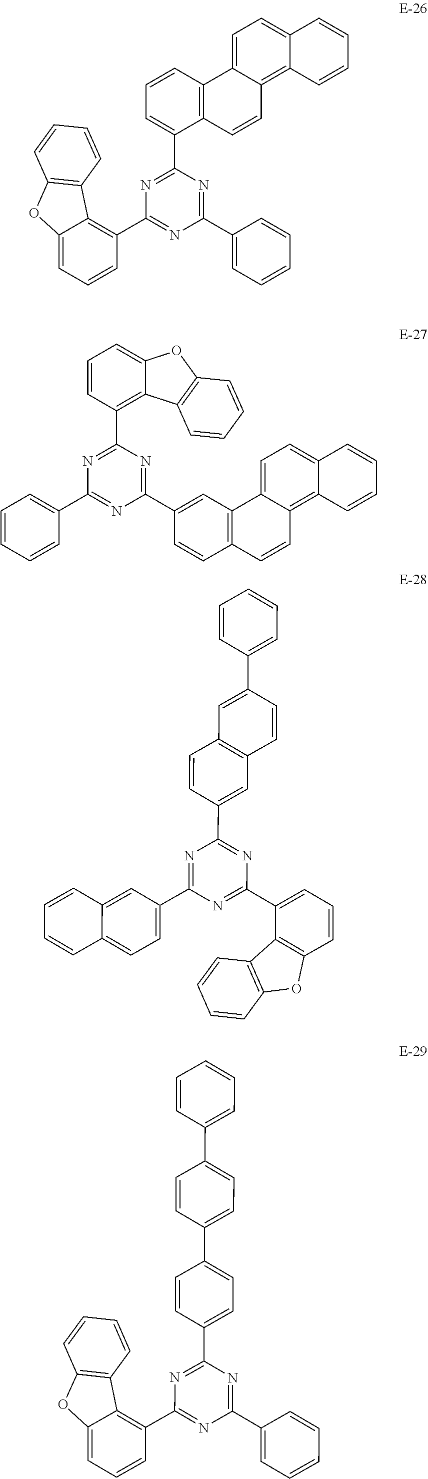 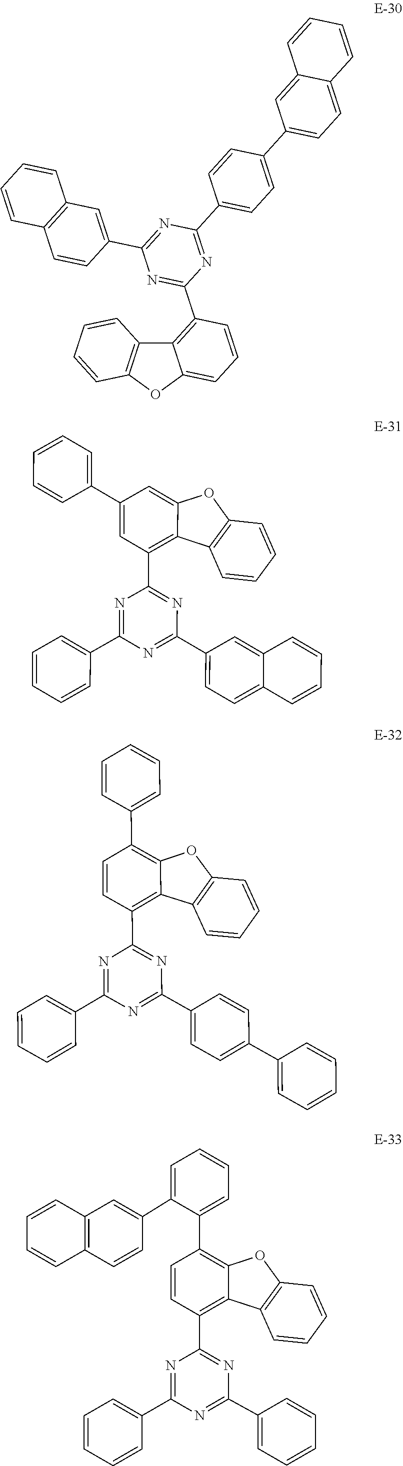 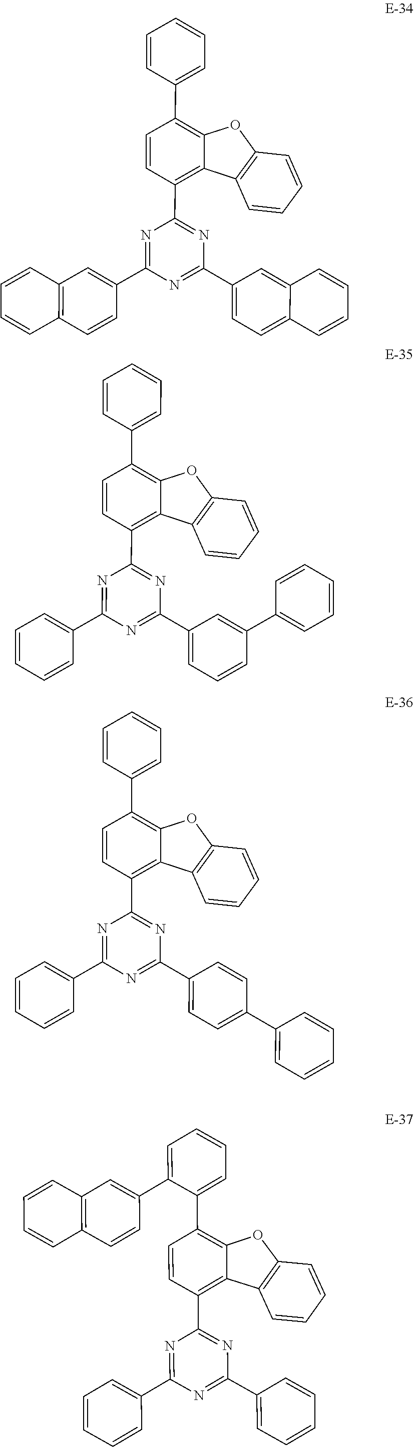 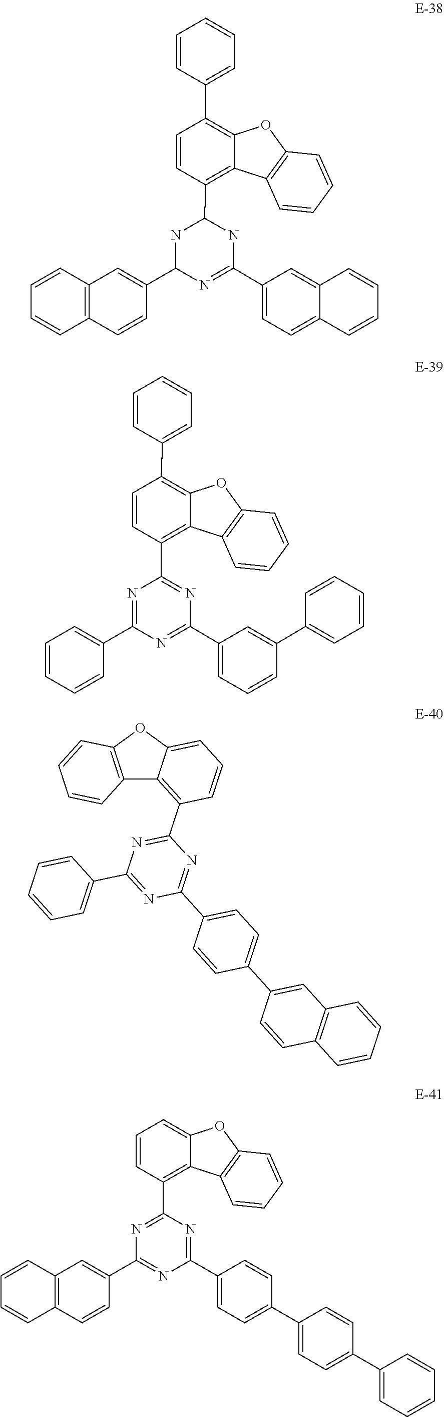 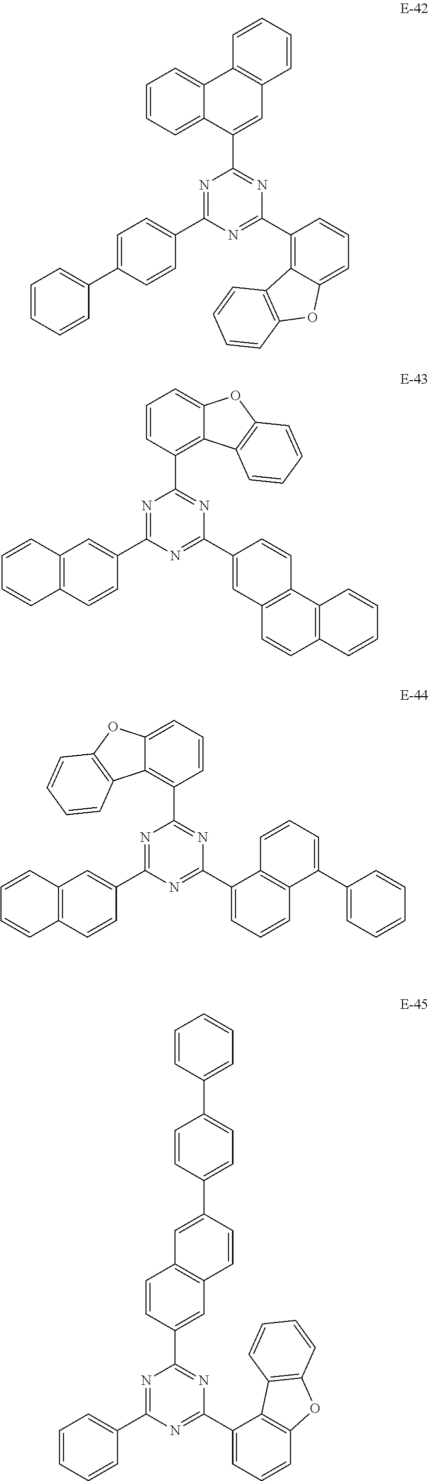 [SMILES: c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4ccc5c(ccc6ccccc65)c4)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1] 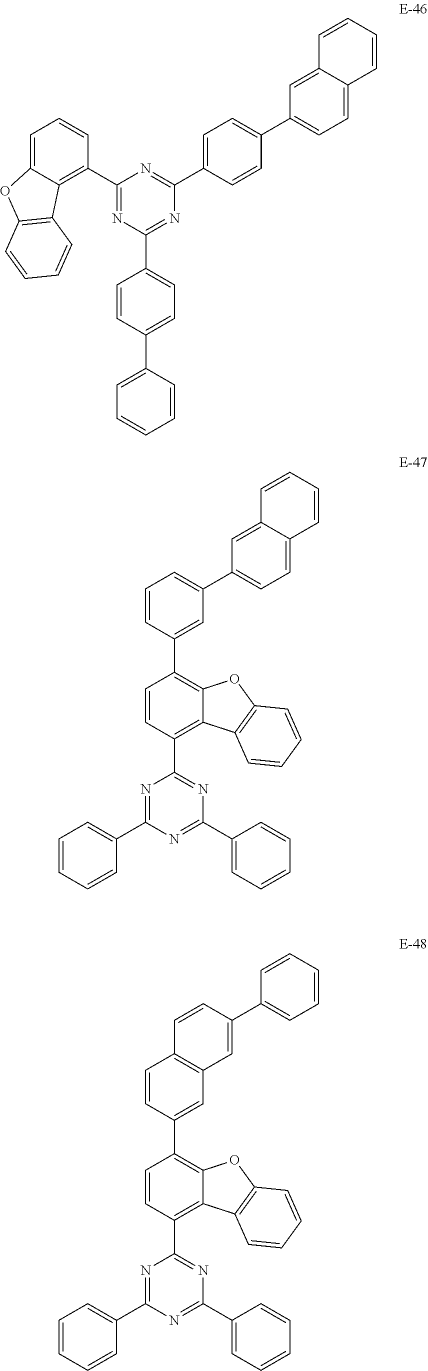 [SMILES: c1ccc(-c2ccc(-c3nc(-c4ccc(-c5ccc6ccccc6c5)cc4)nc(-c4cccc5oc6ccccc6c45)n3)cc2)cc1 ; c1ccc(-c2nc(-c3ccccc3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccccc6)n5)c5c4oc4ccccc45)cc3c2)cc1] 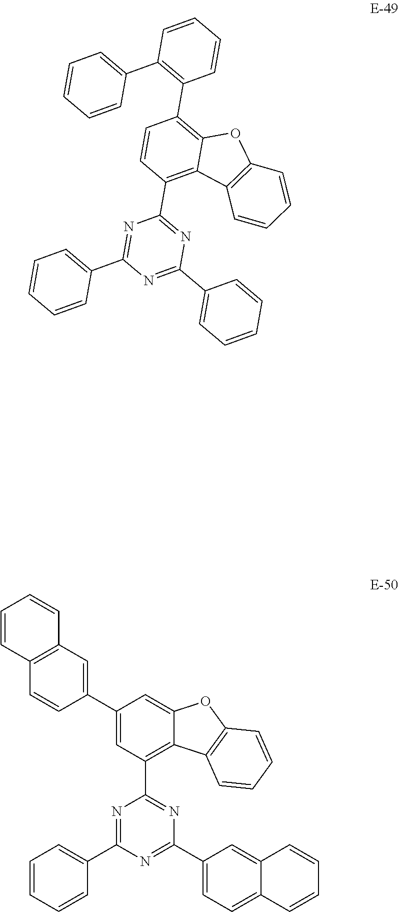 [SMILES: c1ccc(-c2nc(-c3ccccc3)nc(-c3ccc(-c4ccccc4-c4ccccc4)c4oc5ccccc5c34)n2)cc1 ; c1ccc(-c2nc(-c3ccc4ccccc4c3)nc(-c3cc(-c4ccc5ccccc5c4)cc4oc5ccccc5c34)n2)cc1] 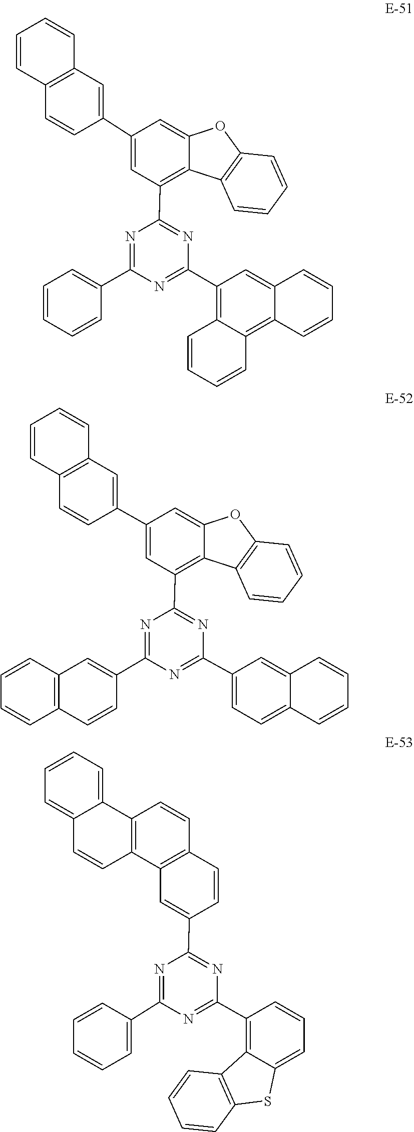 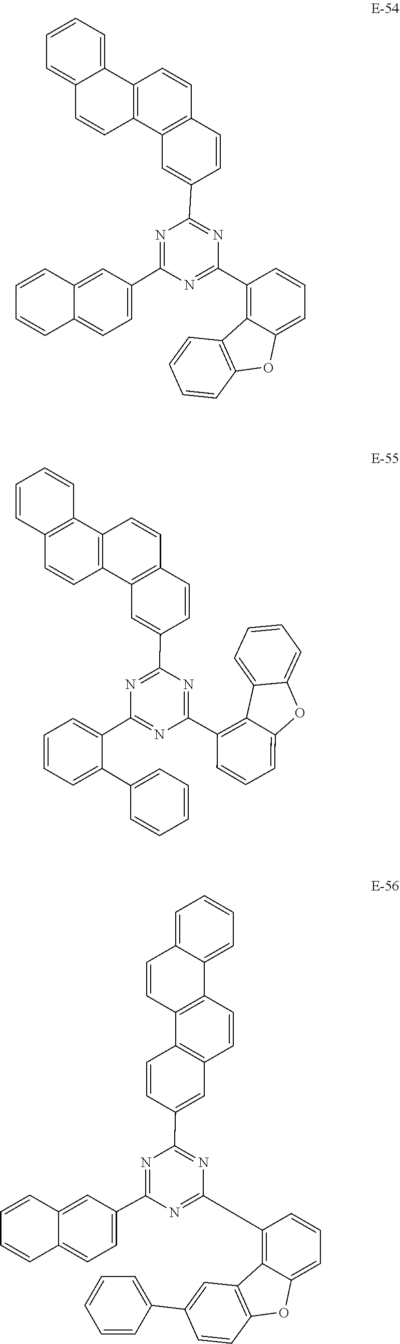 [SMILES: c1ccc2cc(-c3nc(-c4ccc5ccc6c7ccccc7ccc6c5c4)nc(-c4cccc5oc6ccccc6c45)n3)ccc2c1 ; c1ccc(-c2ccccc2-c2nc(-c3ccc4ccc5c6ccccc6ccc5c4c3)nc(-c3cccc4oc5ccccc5c34)n2)cc1 ; c1ccc(-c2ccc3oc4cccc(-c5nc(-c6ccc7ccccc7c6)nc(-c6ccc7c(ccc8c9ccccc9ccc78)c6)n5)c4c3c2)cc1] 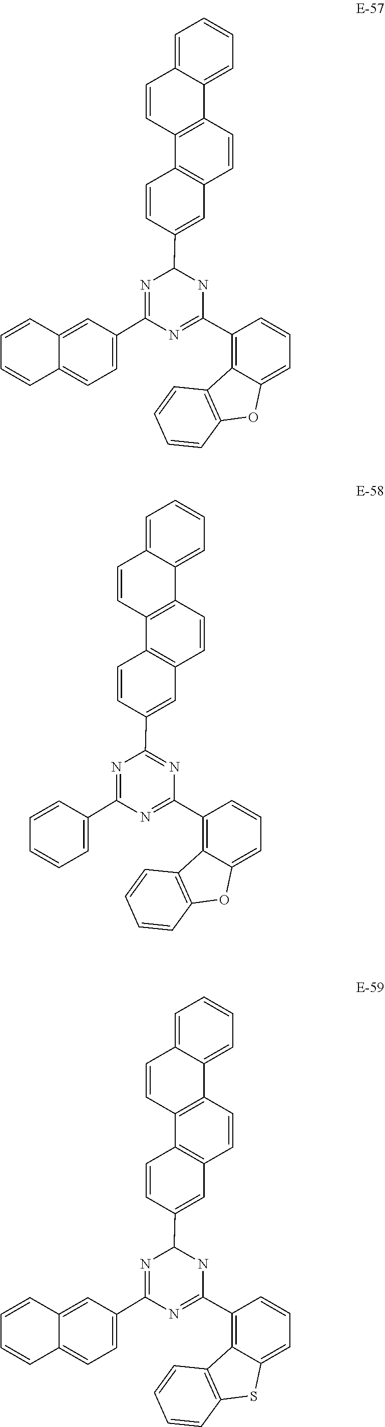 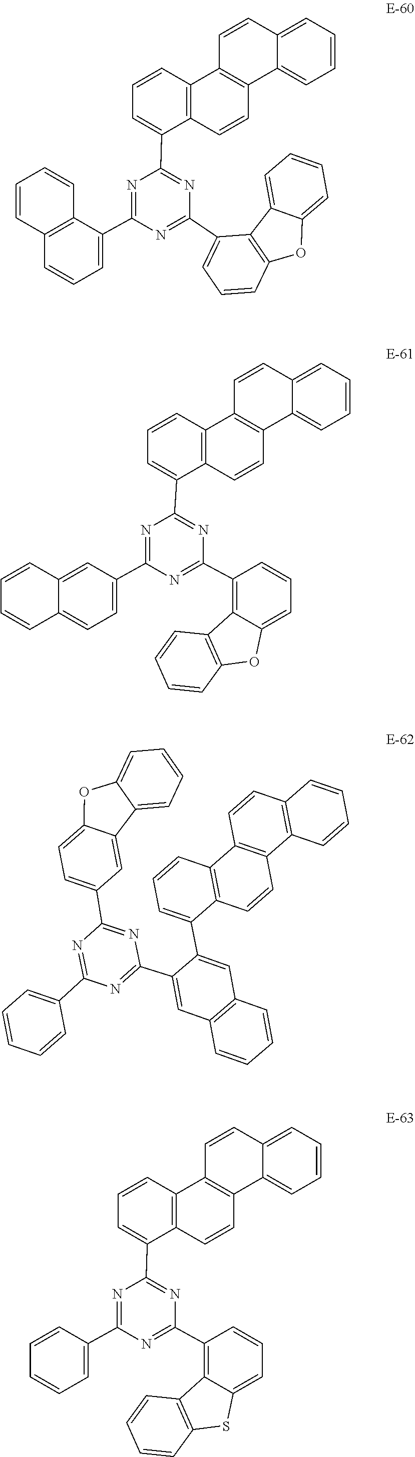 [SMILES: c1ccc2c(-c3nc(-c4cccc5c4ccc4c6ccccc6ccc54)nc(-c4cccc5oc6ccccc6c45)n3)cccc2c1 ; c1ccc2cc(-c3nc(-c4cccc5c4ccc4c6ccccc6ccc54)nc(-c4cccc5oc6ccccc6c45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4sc5ccccc5c34)n2)cc1] 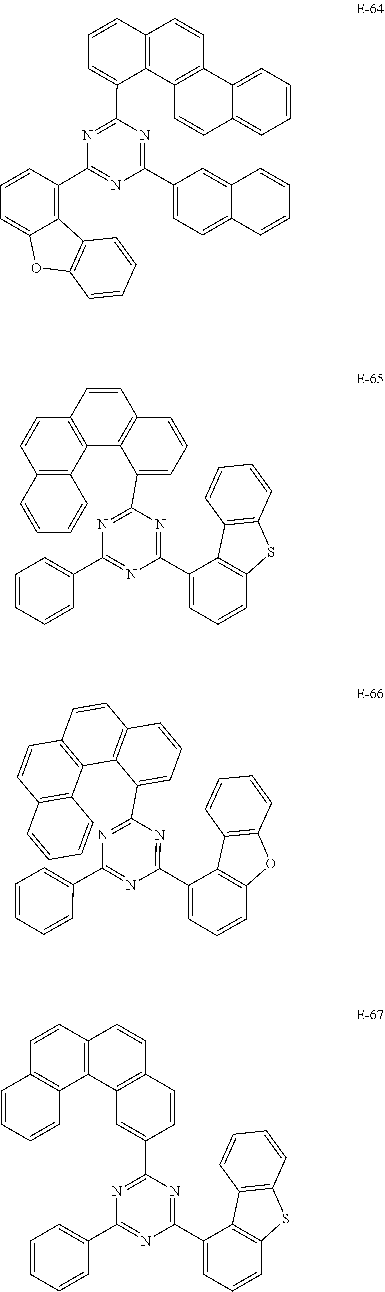 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3ccc4ccc5ccc6ccccc6c5c4c3)nc(-c3cccc4sc5ccccc5c34)n2)cc1] 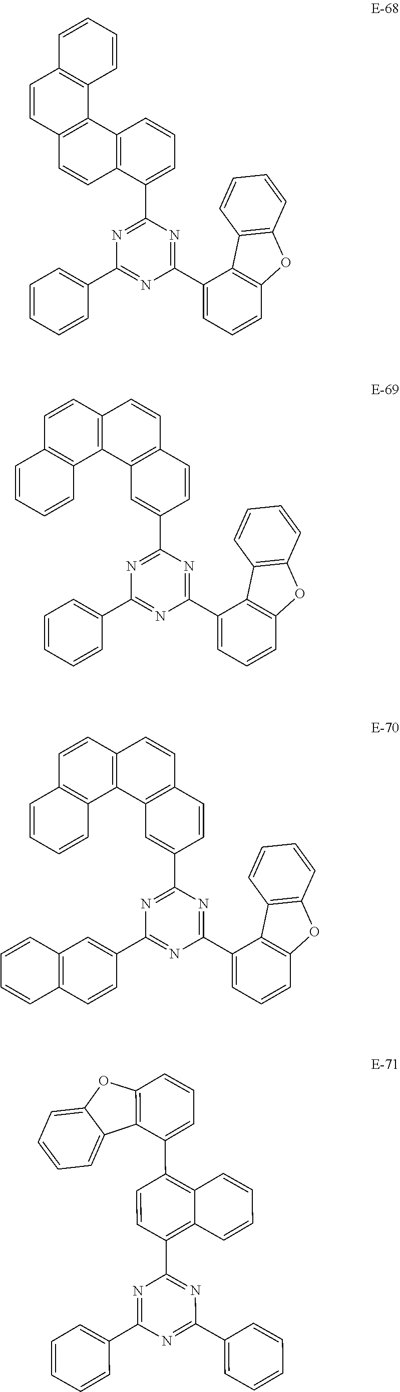 [SMILES: c1ccc(-c2nc(-c3cccc4c3ccc3ccc5ccccc5c34)nc(-c3cccc4oc5ccccc5c34)n2)cc1 ; c1ccc(-c2nc(-c3ccc4ccc5ccc6ccccc6c5c4c3)nc(-c3cccc4oc5ccccc5c34)n2)cc1 ; c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccccc3)nc(-c3ccc(-c4cccc5oc6ccccc6c45)c4ccccc34)n2)cc1] 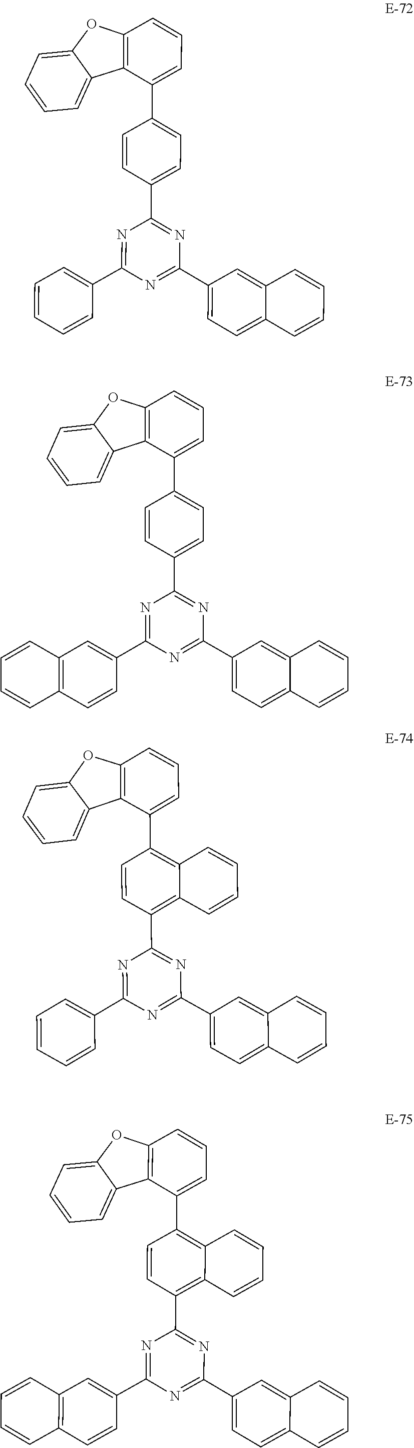 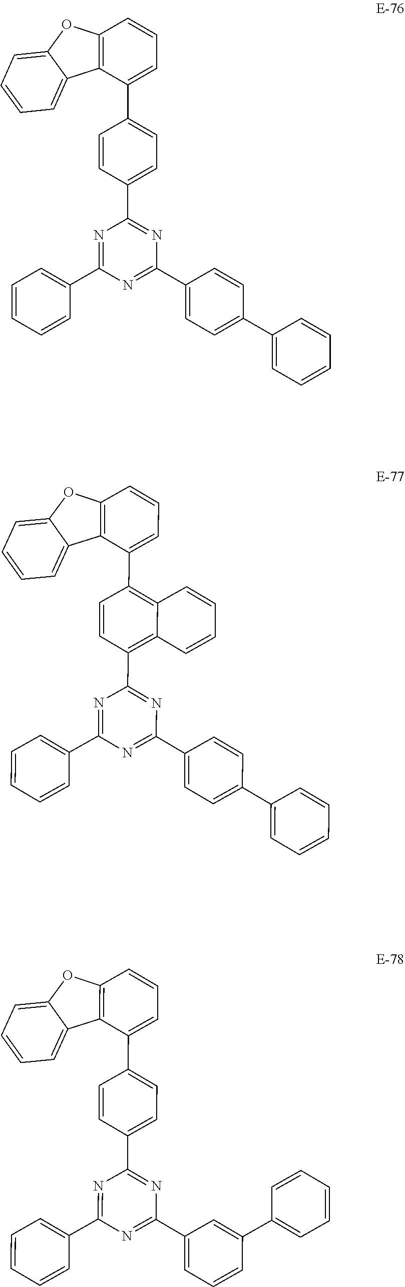 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1] 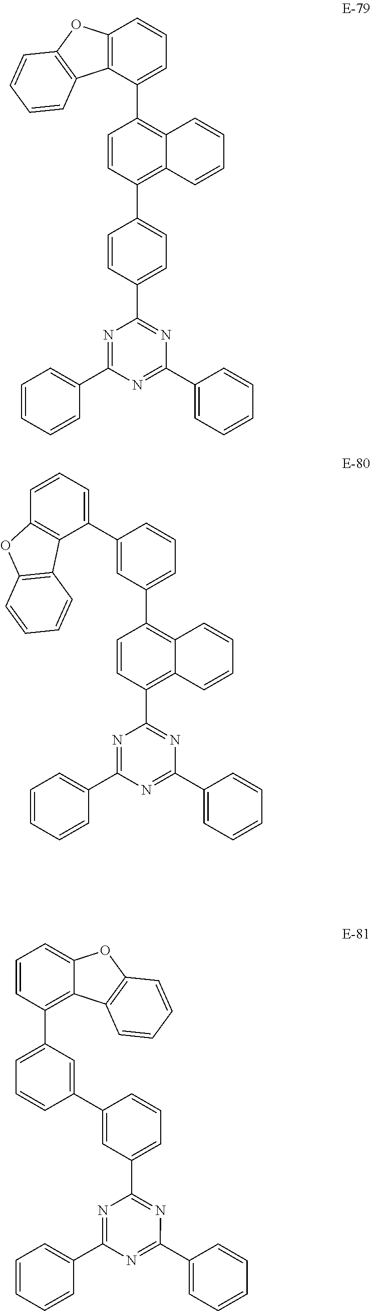 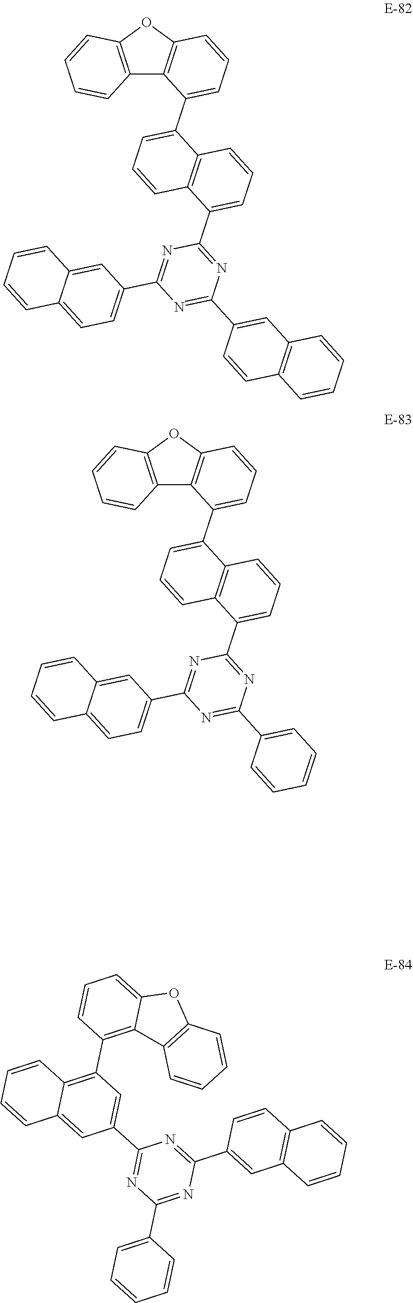 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1] 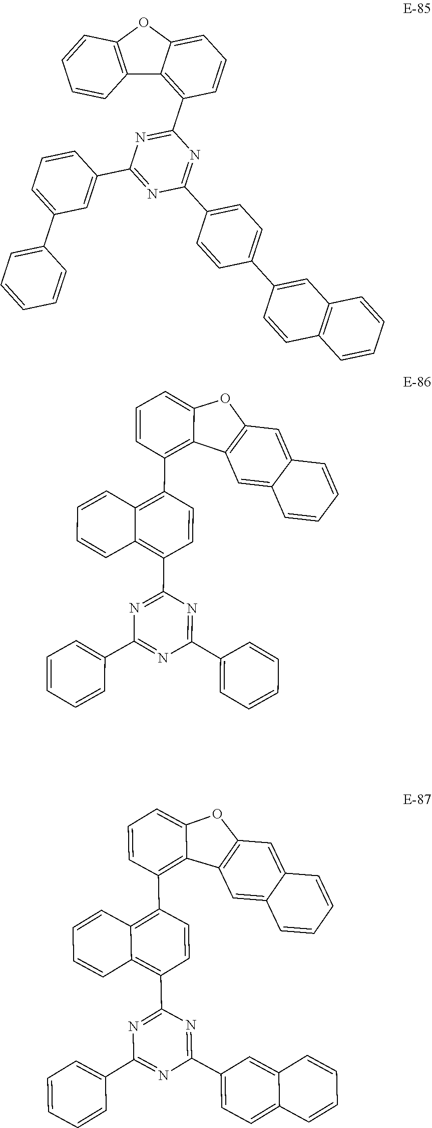 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccccc3)nc(-c3ccc(-c4cccc5oc6cc7ccccc7cc6c45)c4ccccc34)n2)cc1 ; c1ccc(-c2nc(-c3ccc4ccccc4c3)nc(-c3ccc(-c4cccc5oc6cc7ccccc7cc6c45)c4ccccc34)n2)cc1] 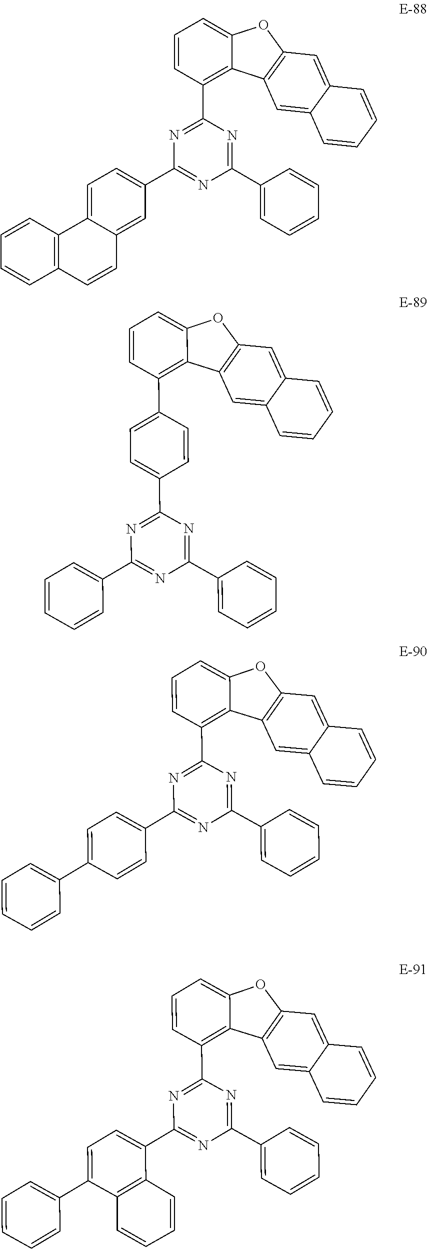 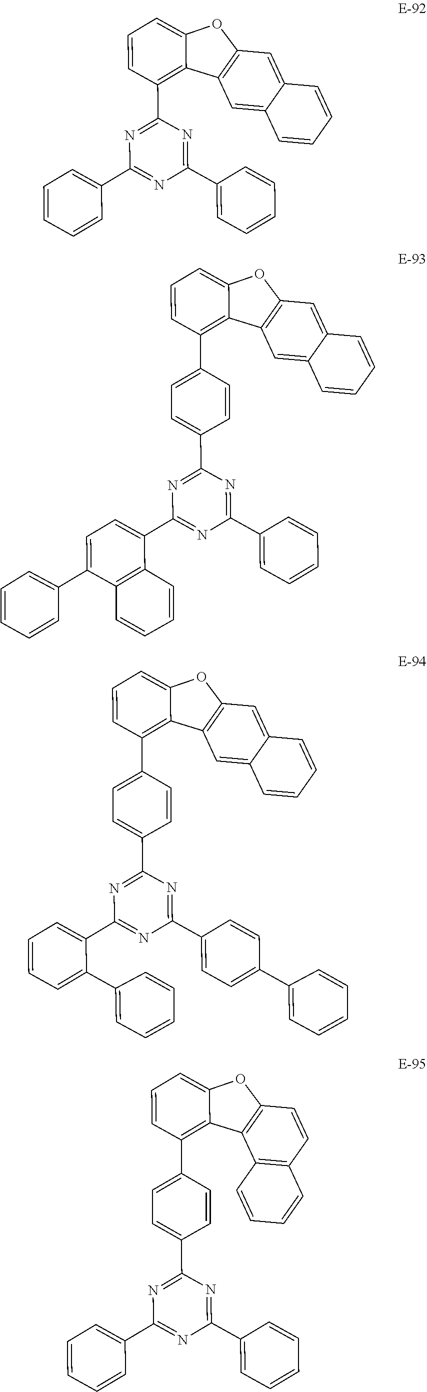 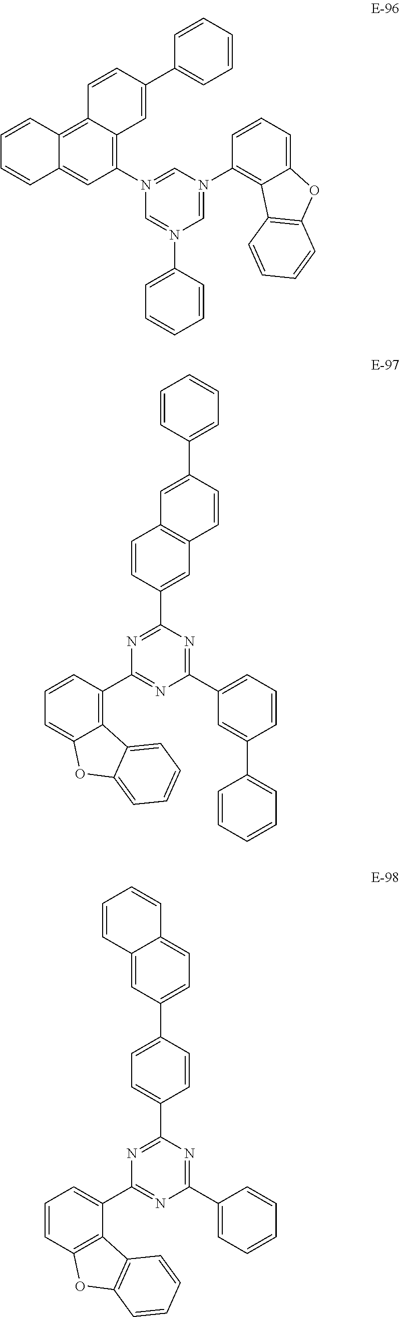 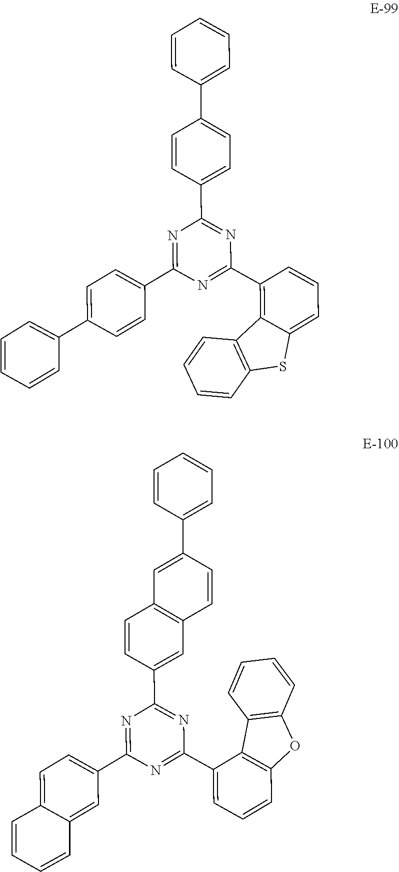 [SMILES: c1ccc(-c2ccc(-c3nc(-c4ccc(-c5ccccc5)cc4)nc(-c4cccc5sc6ccccc6c45)n3)cc2)cc1 ; c1ccc(-c2ccc3cc(-c4nc(-c5ccc6ccccc6c5)nc(-c5cccc6oc7ccccc7c56)n4)ccc3c2)cc1] 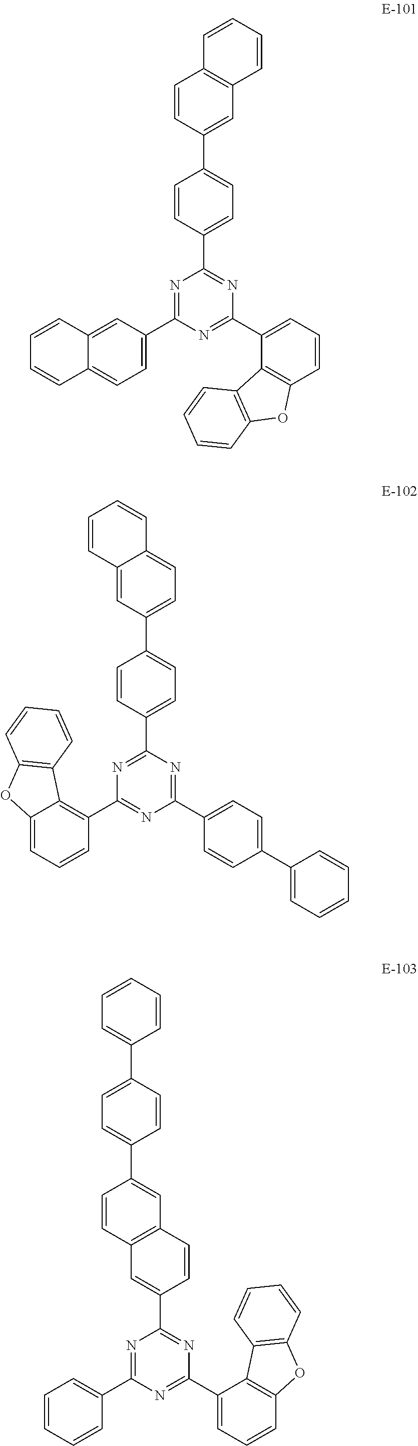 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4cccc5oc6ccccc6c45)n3)cc2)cc1 ; c1ccc(-c2ccc(-c3ccc4cc(-c5nc(-c6ccccc6)nc(-c6cccc7oc8ccccc8c67)n5)ccc4c3)cc2)cc1] 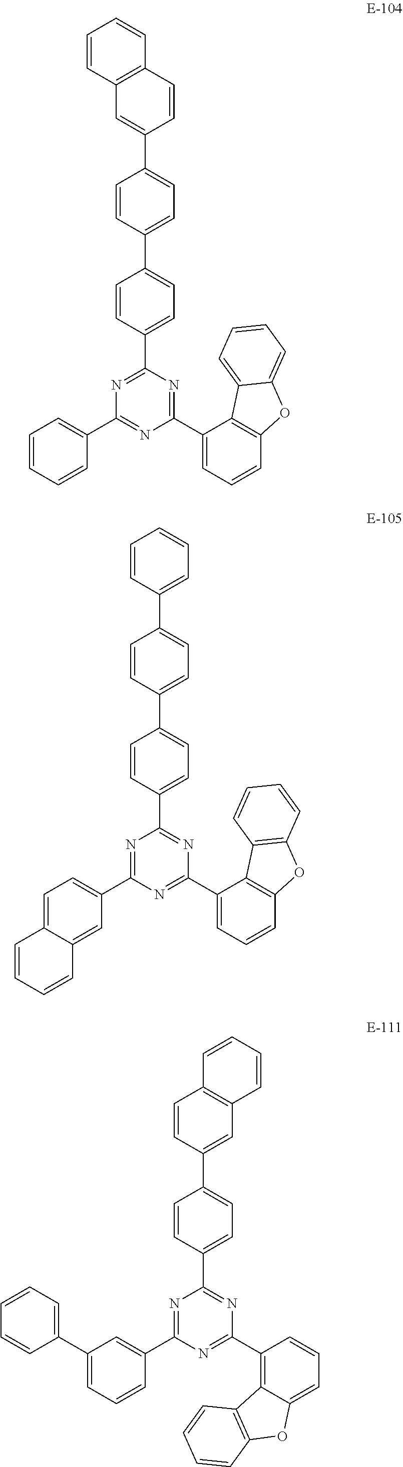 [SMILES: c1ccc(-c2nc(-c3ccc(-c4ccc(-c5ccc6ccccc6c5)cc4)cc3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2cccc(-c3nc(-c4ccc(-c5ccc6ccccc6c5)cc4)nc(-c4cccc5oc6ccccc6c45)n3)c2)cc1] 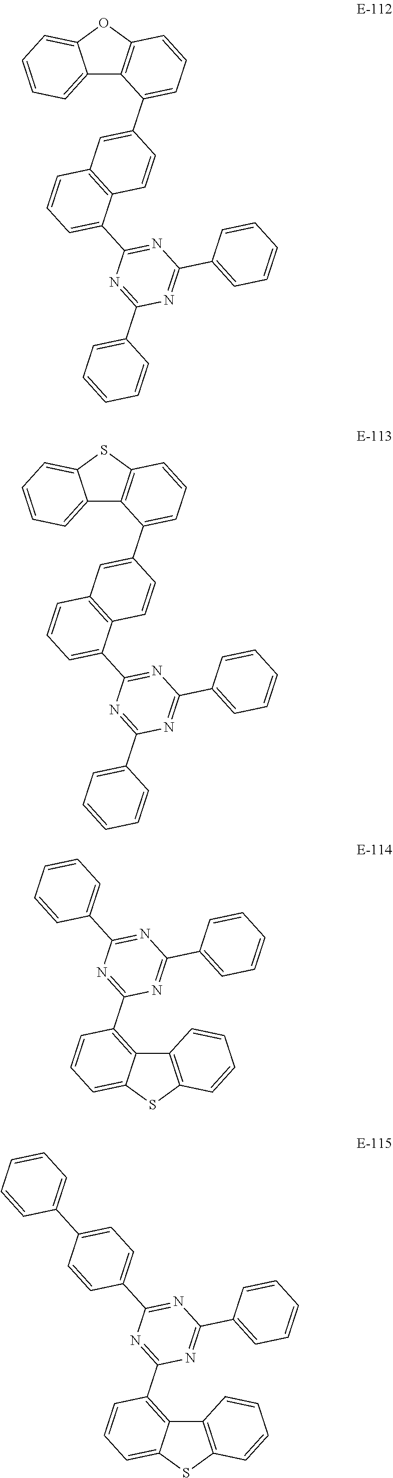 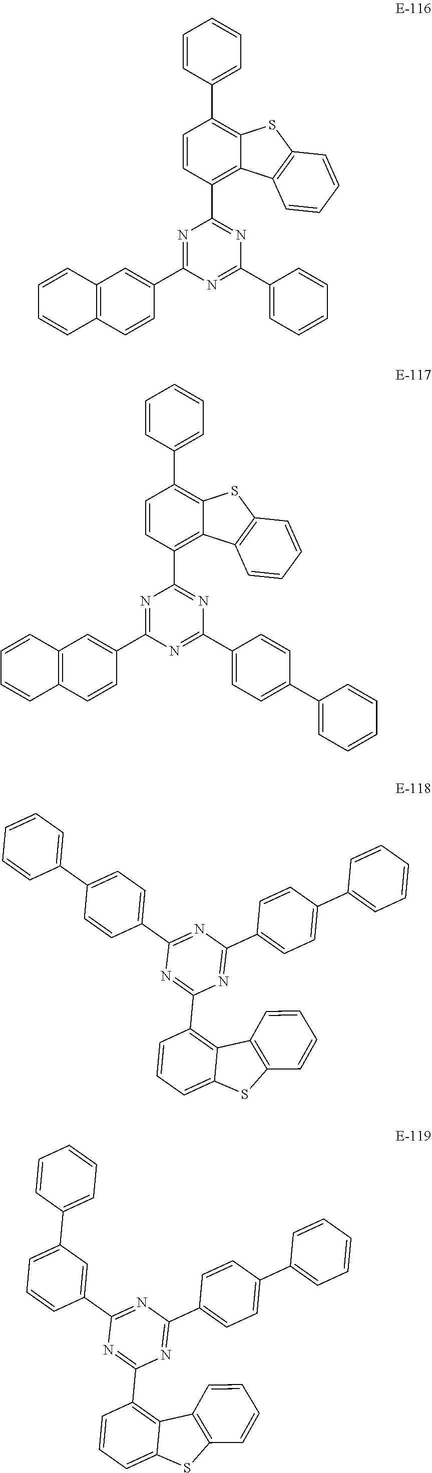 [SMILES: c1ccc(-c2nc(-c3ccc4ccccc4c3)nc(-c3ccc(-c4ccccc4)c4sc5ccccc5c34)n2)cc1 ; c1ccc(-c2ccc(-c3nc(-c4ccc5ccccc5c4)nc(-c4ccc(-c5ccccc5)c5sc6ccccc6c45)n3)cc2)cc1 ; c1ccc(-c2ccc(-c3nc(-c4ccc(-c5ccccc5)cc4)nc(-c4cccc5sc6ccccc6c45)n3)cc2)cc1 ; c1ccc(-c2ccc(-c3nc(-c4cccc(-c5ccccc5)c4)nc(-c4cccc5sc6ccccc6c45)n3)cc2)cc1] 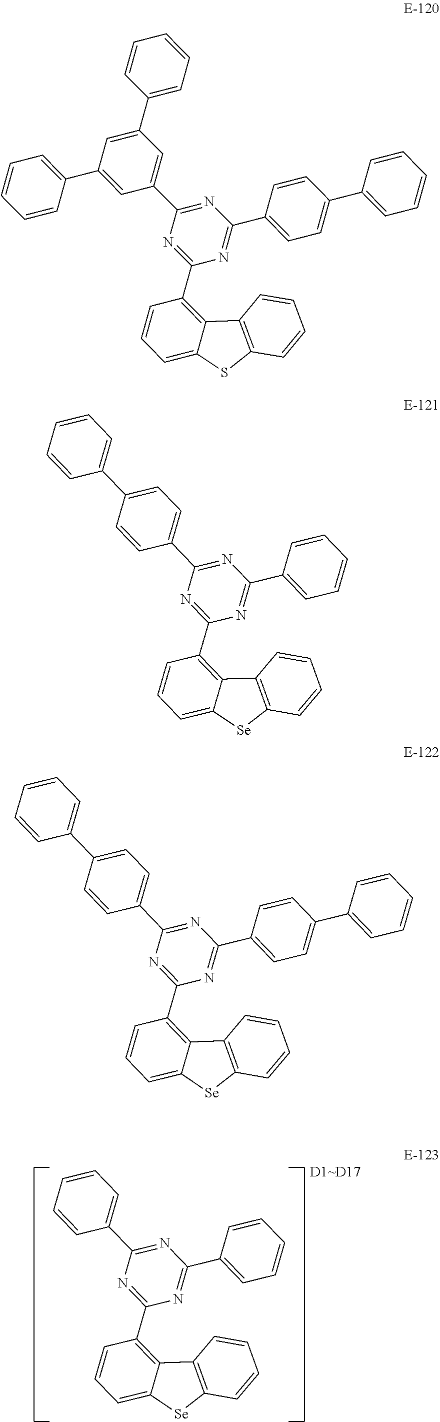 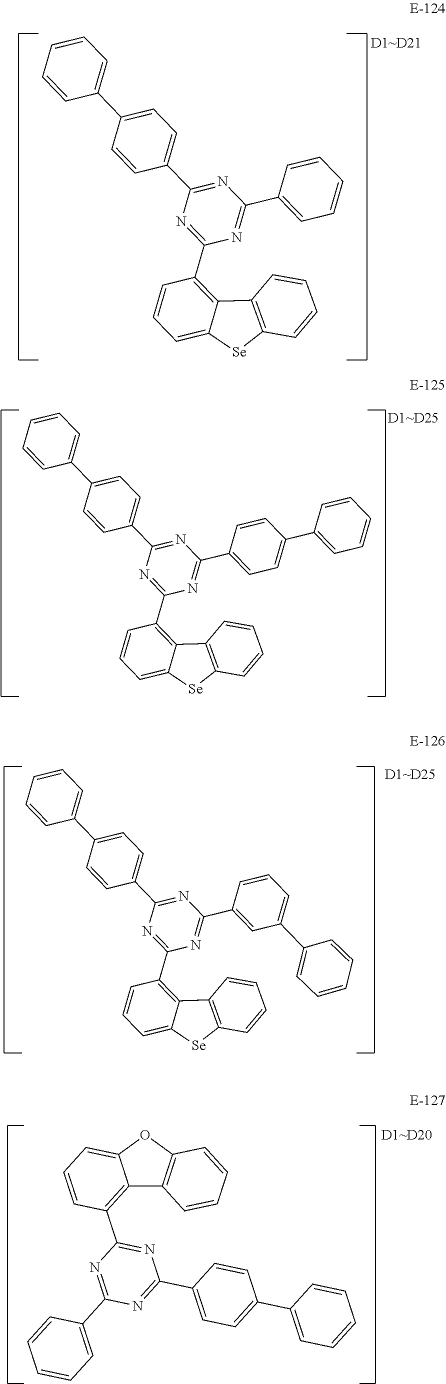 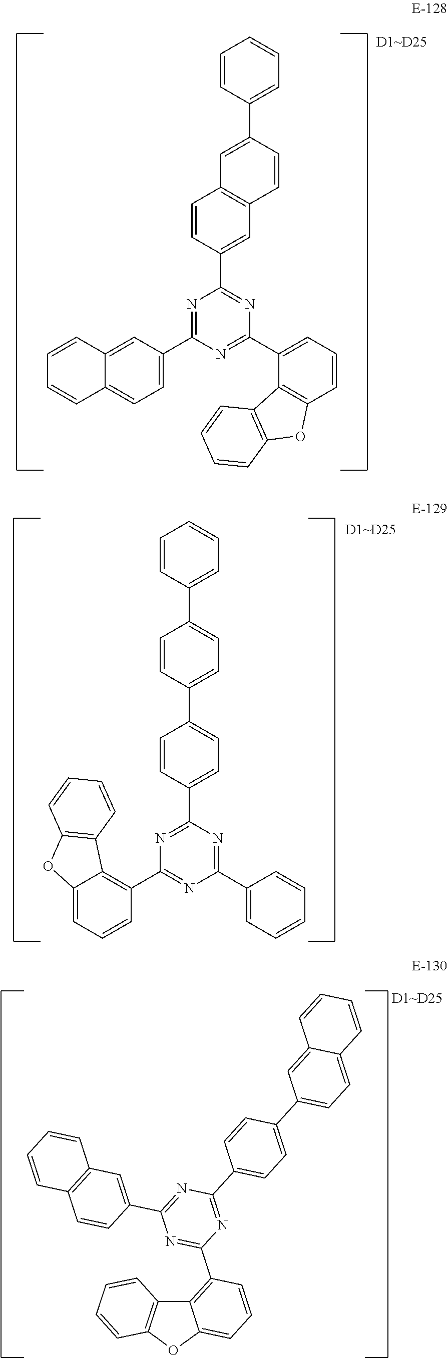 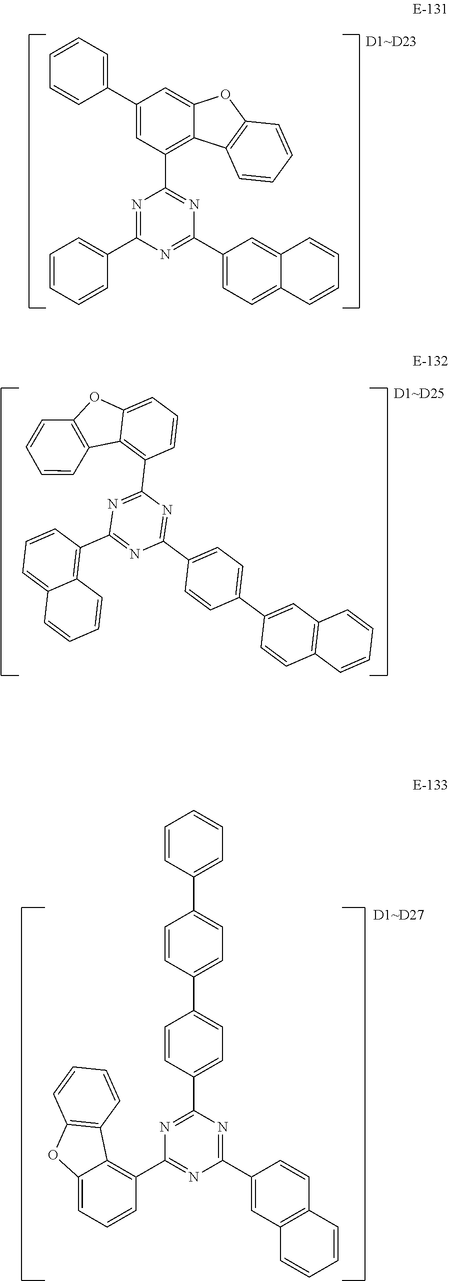 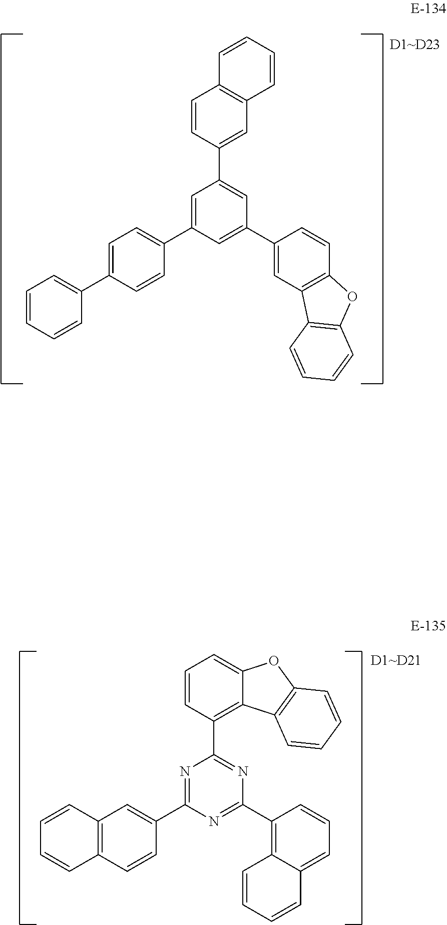 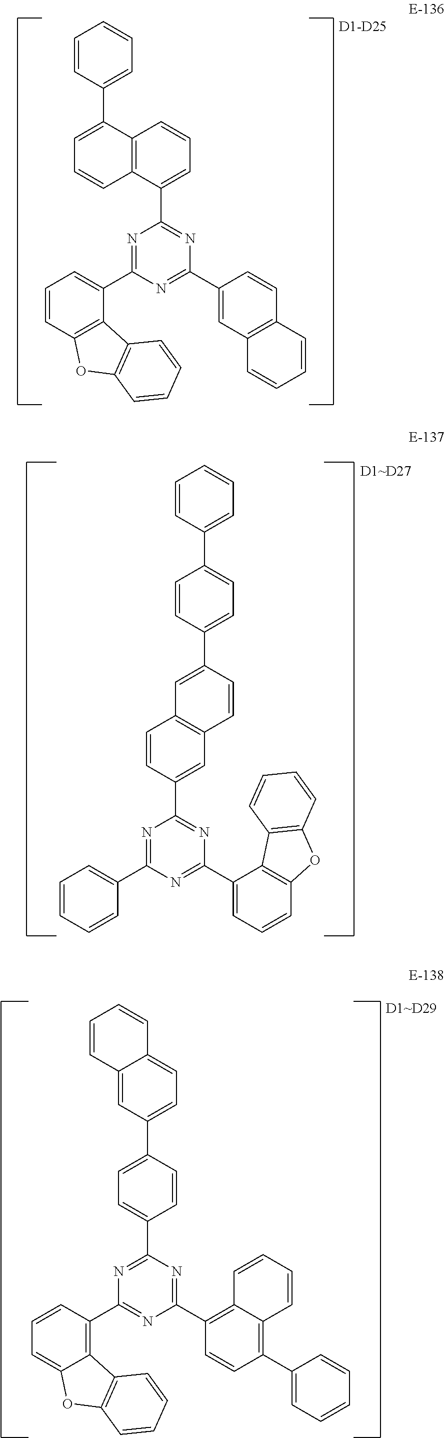 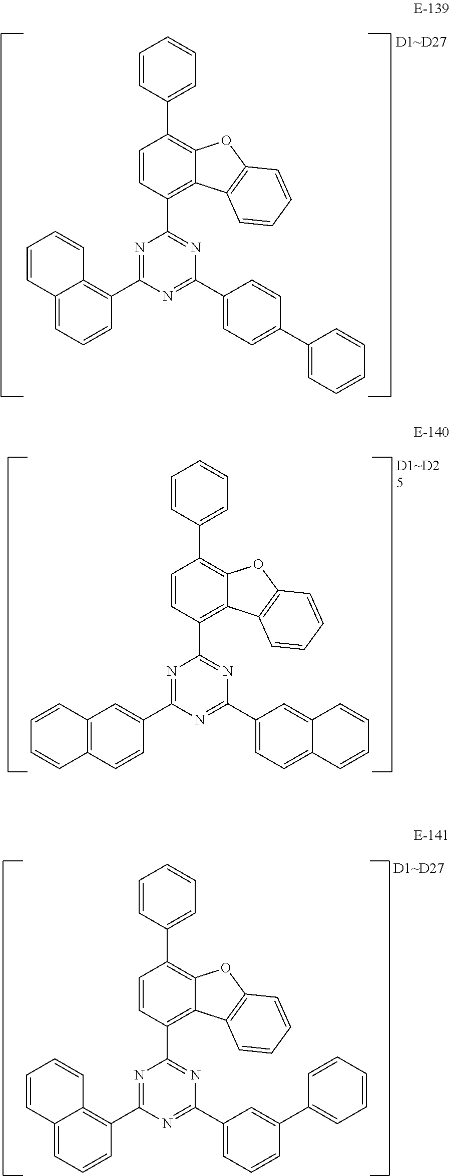 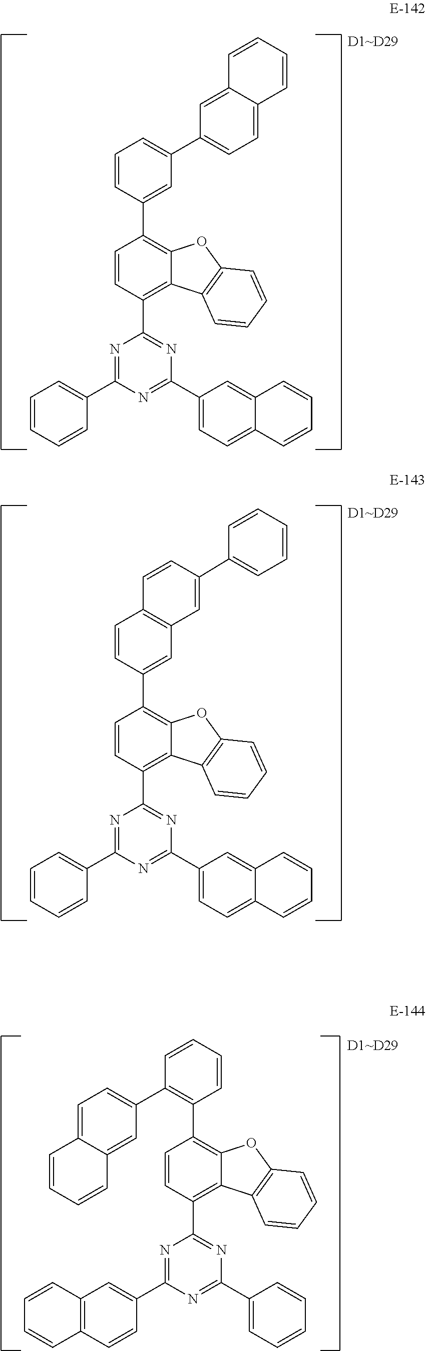 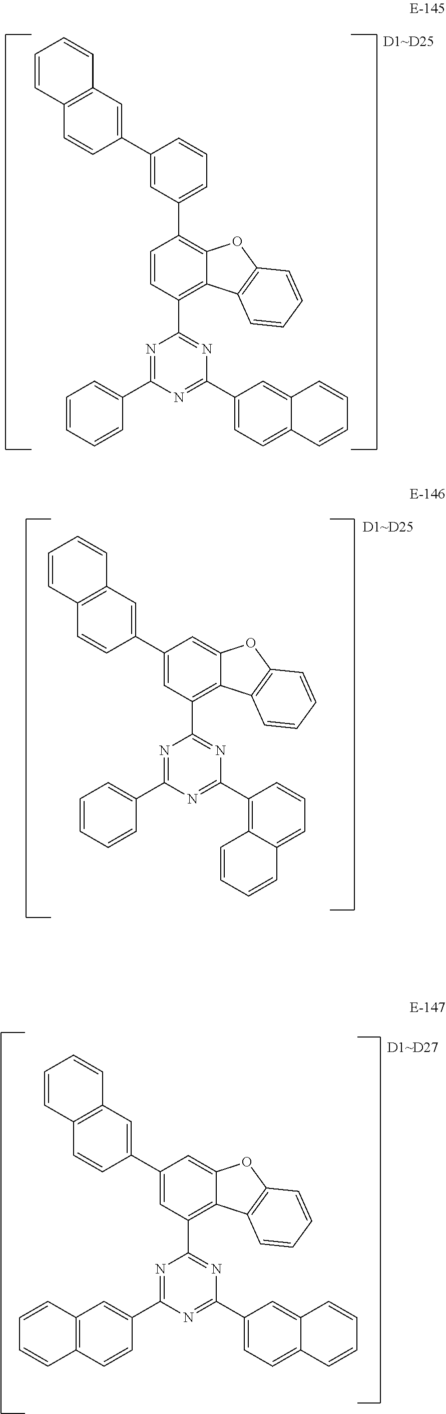 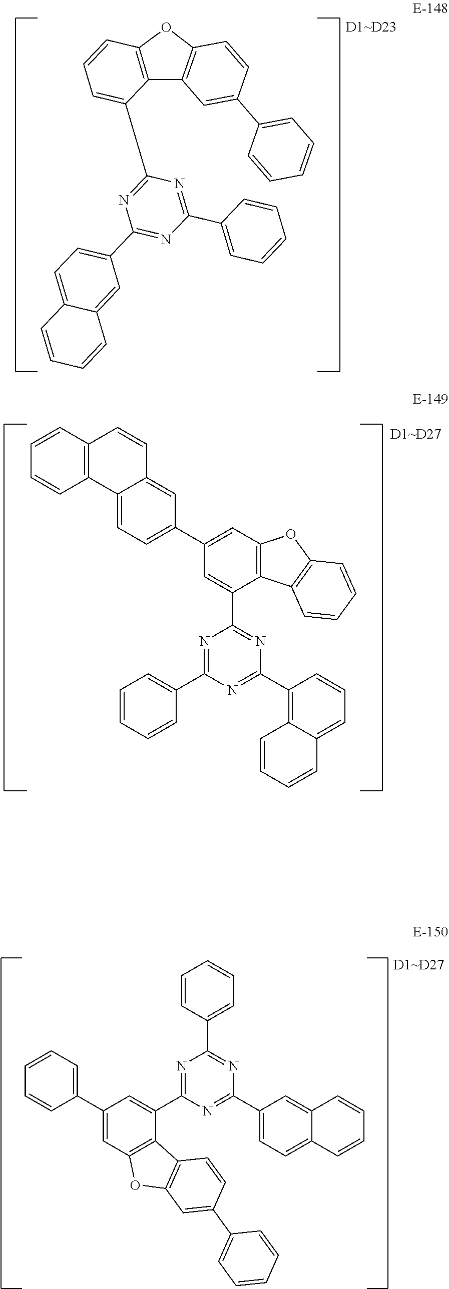 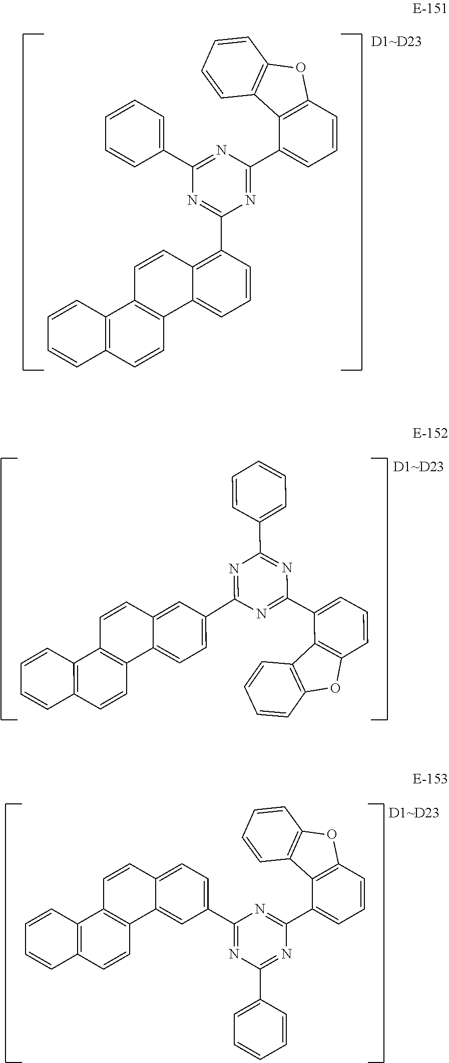 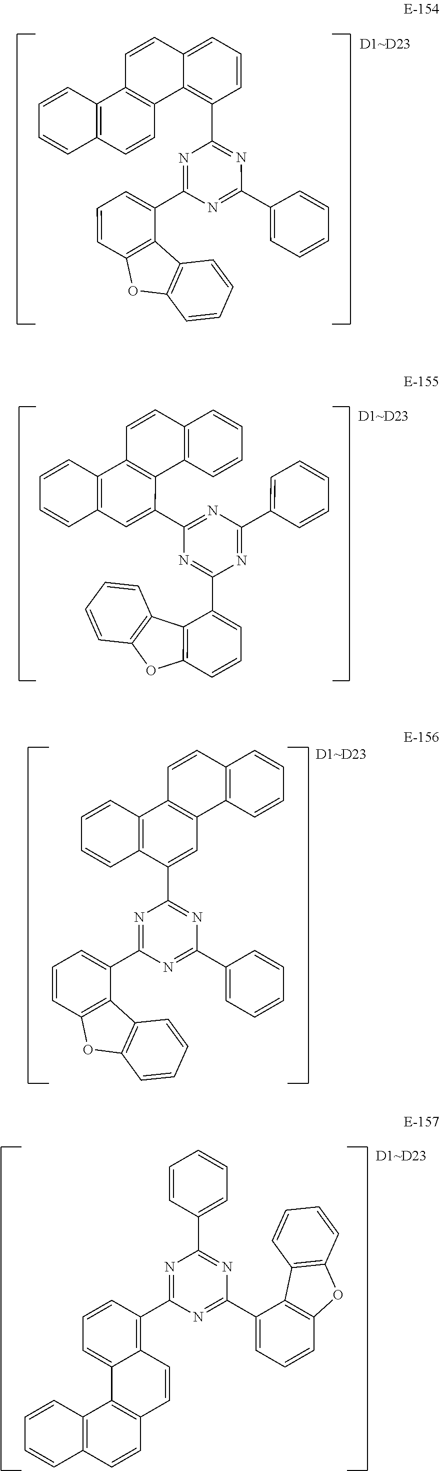 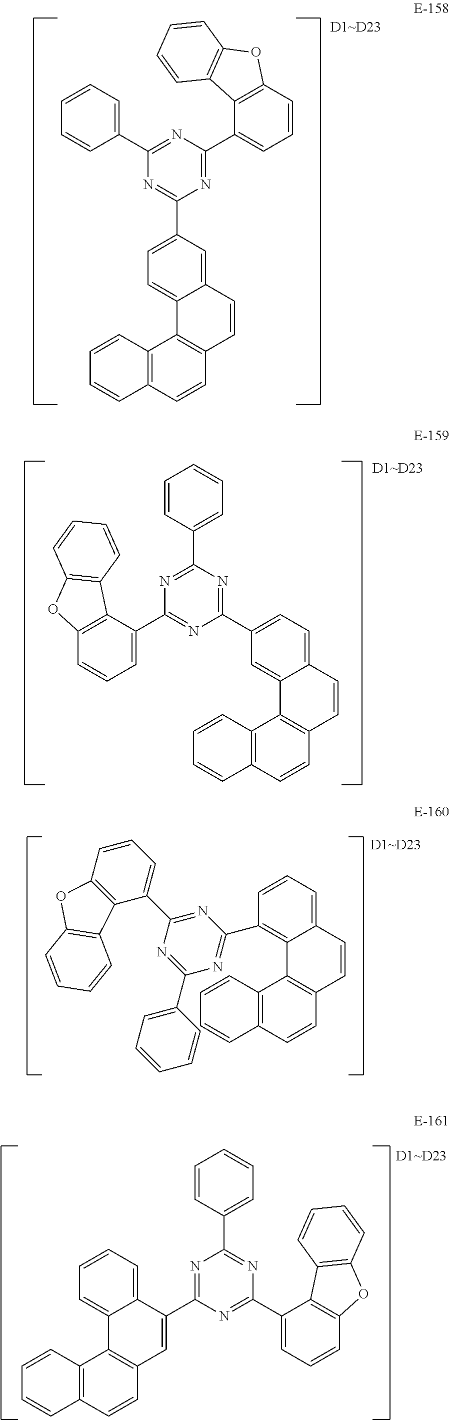 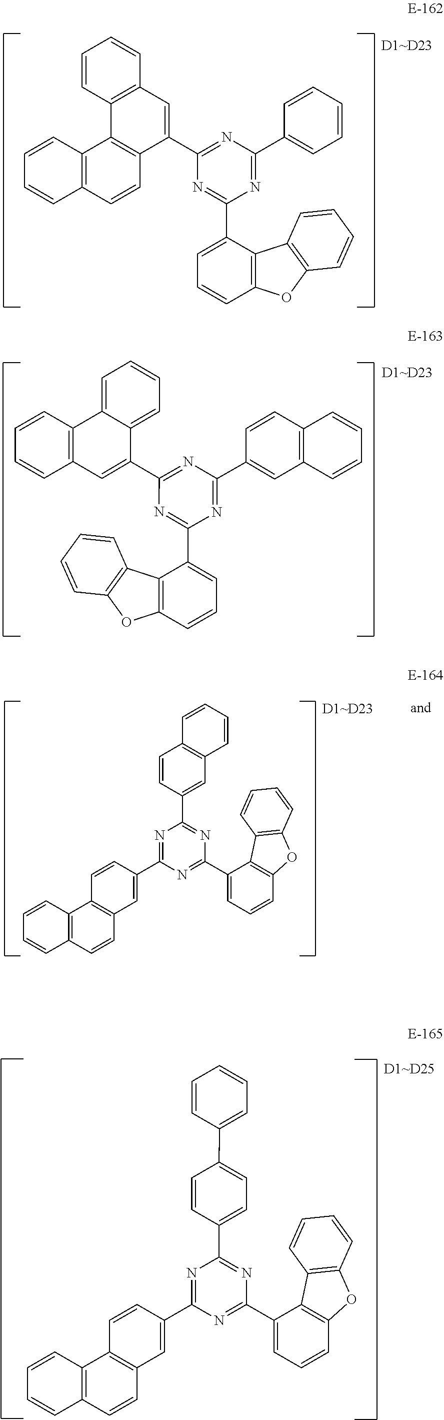
In the compounds above, the “n” in Dn refers to the number of deuteriums that can be substituted in the compound.

8. An organic electroluminescent device comprising a first electrode; a second electrode; and at least one light-emitting layer(s) between the first electrode and the second electrode, wherein the at least one of the light-emitting layer(s) comprises the plurality of host materials according to claim 1.
